(19) United States
(12) Patent Application Publication
(10) Pub. No.: US 2026/0216368 A1
(43) Pub. Date: Jul. 30, 2026

(54) BIS-ESTER AND AMIDE CATIONIC LIPIDS

(71) Applicant: SANOFI PASTEUR INC., Swiftwater, PA (US)

(72) Inventors: Ramesh DASARI, Cambridge, MA (US); Hongfeng DENG, Cambridge, MA (US); Saswata KARMAKAR, Cambridge, MA (US); Lingyao LI, Cambridge, MA (US); Apiwat WANGWEERAWONG, Cambridge, MA (US)

(21) Appl. No.: 19/141,841

(22) PCT Filed: Dec. 22, 2023
(86) PCT No.: PCT/EP2023/087539

§ 371 Date: Jun. 20, 2025

(30) Foreign Application Priority Data
Dec. 22, 2022 (EP) 22307007.9

(51) Int. Cl. A61K 48/00; A61K 47/18; C07C 229/22

(52) U.S. Cl. CPC A61K 48/0033; A61K 47/18; C07C 229/22

(57) ABSTRACT

The present invention provides, in part, bis-ester and amide cationic lipid compounds of Formula (I): or a pharmaceutically acceptable salt thereof, bis-ester and amide cationic lipid compounds of Formula (II): or a pharmaceutically acceptable salt thereof, bis-ester and amide cationic lipid compounds of Formula (III): or a pharmaceutically acceptable salt thereof, and bis-ester and amide cationic lipid compounds of Formula (IV): or a pharmaceutically acceptable salt thereof. The compounds provided herein can be useful for delivery and expression of mRNA and encoded protein, e.g., as a component of liposomal delivery vehicle, and accordingly can be useful for treating various diseases, disorders and conditions, such as those associated with deficiency of one or more proteins.

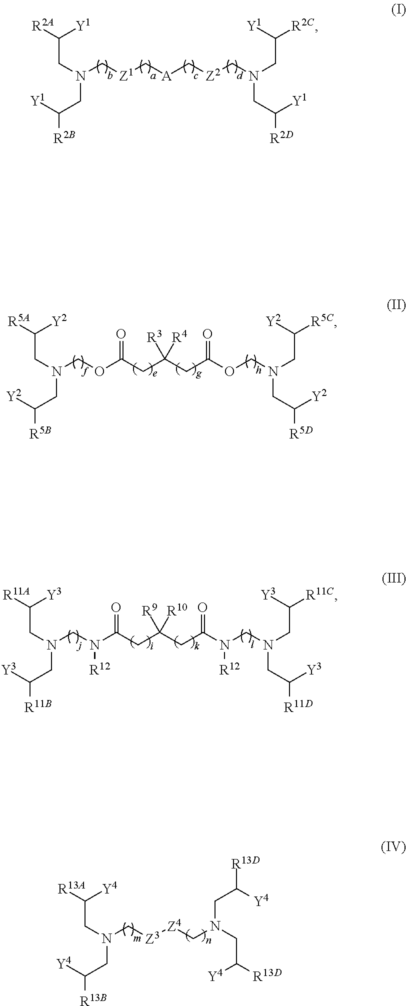

### CROSS-REFERENCE TO RELATED APPLICATIONS

The present application claims priority to European Application No. 22307007.9, filed Dec. 22, 2022, which is incorporated by reference in its entirety.

### BACKGROUND

Delivery of nucleic acids has been explored extensively as a potential therapeutic option for certain disease states. In particular, messenger RNA (mRNA) therapy has become an increasingly important option for the prevention and treatment of various diseases (e.g. in the use of vaccines).

Efficient delivery of liposome-encapsulated nucleic acids remains an active area of research. The cationic lipid component of liposomes encapsulating nucleic acids plays an important role in facilitating effective encapsulation of the nucleic acid during the loading of liposomes. In addition, cationic lipids may play an important role in the efficient release of the nucleic acid cargo from the liposome into the cytoplasm of a target cell. Various cationic lipids suitable for in vivo use have been discovered. However, there remains a need to identify lipids that can be synthesized efficiently and cheaply without the formation of potentially toxic by-products.

There are cationic lipids that contain a cyclic ring structure as a central core (lipidoids like cKK-E12—structure shown below):

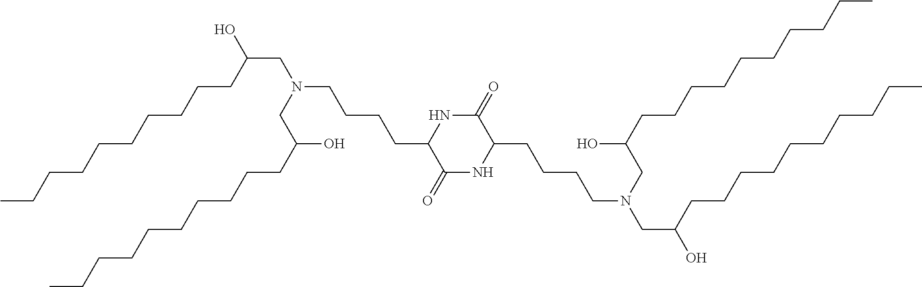 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

The inventors of the present invention have surprisingly found that cationic lipids made from commercially available acyclic linkers (such as tartronic acid and aminomalonic acid) have high levels of peptide or protein expression when delivering mRNA encoding said peptide or protein while having a reduced size and complexity. This reduced size and complexity allows for the development of new lipid analogues more speedily and the claimed cationic lipids are also advantageous when it comes to downstream scale up and manufacturing as compared to earlier lipidoid cationic lipids.

### SUMMARY OF THE INVENTION

The present invention provides, among other things, a novel class of cationic lipid compounds for in vivo delivery of therapeutic agents, such as nucleic acids. It is contemplated that these compounds are capable of highly effective in vivo delivery of the therapeutic agents and vaccines while maintaining a favorable safety profile. Lipid nanoparticles comprising the cationic lipids of the present invention (e.g. compounds LXXIII and LXXIV) also exhibit enhanced thermostability, which is beneficial for the development of the corresponding therapeutic agents and vaccines.

The cationic lipids of the present invention comprise cleavable groups (e.g., esters and disulphides) that are contemplated to improve biodegradability and thus contribute to their favorable safety profile.

In an aspect, provided herein are cationic lipids having a structure according to Formula (I):

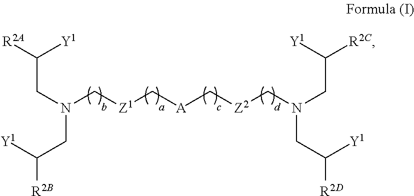

or a pharmaceutically acceptable salt thereof,
wherein A is selected from —N(R¹)— or —S—S—;
R¹ is optionally substituted (C₁-C₆)alkyl;
a and c are integers that are each independently selected from 1, 2, 3 or 4;
b and d are integers that are each independently selected from 1, 2, 3, 4, 5 or 6;

Z¹ is selected from a covalent bond,

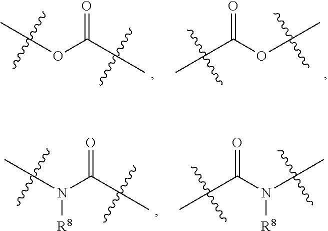

or —S—S—, wherein the left hand side of each depicted structure is bound to the —(CH₂)_{b}—;
Z² is selected from a covalent bond,

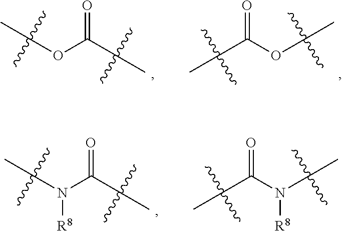

or —S—S—, wherein the right hand side of each depicted structure is bound to the —(CH₂)_{d}—;
each Y¹ is independently selected from hydrogen or —OH;
each R³ is independently selected from hydrogen or optionally substituted (C₁-C₆)alkyl;
R^{2A}, R^{2B}, R^{2C}, and R^{2D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y¹)— when W¹ is a covalent bond.

In an aspect, provided herein are cationic lipids having a structure according to Formula (II):

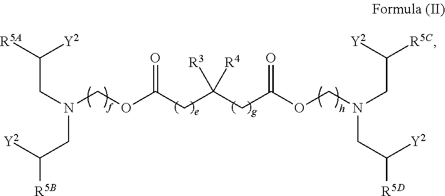

or a pharmaceutically acceptable salt thereof,
wherein R³ is selected from hydrogen, or optionally substituted (C₁-C₆)alkyl;
R⁴ is selected from hydrogen, —OH, —NH₂, optionally substituted (C₁-C₆)alkyl, optionally substituted aryl, optionally substituted heteroaryl, optionally substituted (C₁-C₃)alkylene-optionally substituted aryl, or optionally substituted (C₁-C₃)alkylene-optionally substituted heteroaryl;
e and g are integers that are each independently selected from 0, 1, 2, 3, or 4;
f and h are integers that are each independently selected from 1, 2, 3, 4, 5 or 6; each Y² is independently selected from hydrogen or —OH;
R^{5A}, R^{5B}, R^{5C}, and R^{5D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y²)— when W¹ is a covalent bond.

In an aspect, provided herein are cationic lipids having a structure according to Formula (III):

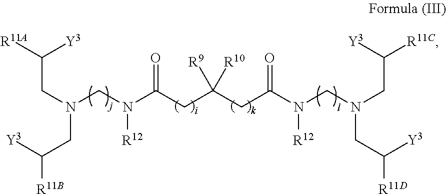

or a pharmaceutically acceptable salt thereof,
wherein R⁹ is selected from hydrogen, or optionally substituted (C₁-C₆)alkyl;
R¹⁰ is selected from hydrogen, —OH, —NH₂, optionally substituted (C₁-C₆)alkyl, optionally substituted aryl, optionally substituted heteroaryl, optionally substituted (C₁-C₃)alkylene-optionally substituted aryl, or optionally substituted (C₁-C₃)alkylene-optionally substituted heteroaryl;
i and k are integers that are each independently selected from 0, 1, 2, 3, or 4;
j and l are integers that are each independently selected from 1, 2, 3, 4, 5 or 6;
each Y³ is independently selected from hydrogen or —OH;
each R¹² is independently selected from hydrogen or optionally substituted (C₁-C₆)alkyl;
R^{11A}, R^{11B}, R^{11C}, and R^{11D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C1-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y³)— when W¹ is a covalent bond.

In an aspect, provided herein are cationic lipids having a structure according to Formula (IV):

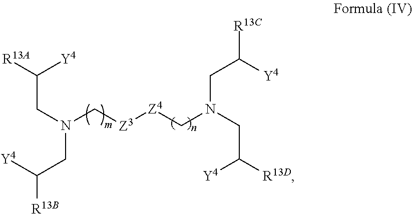

or a pharmaceutically acceptable salt thereof,
wherein
m and n are integers that are each independently selected from 1, 2, 3, 4, 5 or 6;
Z³ is an aromatic amino acid residue, wherein the α-carbon carboxyl group (—C(═O)—O—) of the aromatic amino acid residue is bound to the —(CH₂)ₘ- and the α-carbon aminyl group (—NH—) of the aromatic amino acid residue is bound to the Z⁴;
Z⁴ is selected from

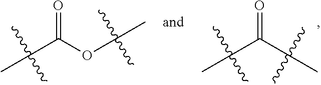

wherein the right hand side of each depicted structure is bound to the —(CH₂)ₙ—;
each Y⁴ is independently selected from hydrogen or —OH;
R^{13A}, R^{13B}, R^{13C} and R^{13D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y⁴)— when W¹ is a covalent bond.

In an aspect, provided herein are cationic lipids that are pharmaceutically acceptable salts of Formula (I).

In an aspect, provided herein are cationic lipids that are pharmaceutically acceptable salts of Formula (II).

In an aspect, provided herein are cationic lipids that are pharmaceutically acceptable salts of Formula (III).

In an aspect, provided herein are cationic lipids that are pharmaceutically acceptable salts of Formula (IV).

In an aspect, provided herein are compositions comprising the cationic lipid of the present invention or a pharmaceutically acceptable salt thereof, one or more non-cationic lipids, one or more cholesterol-based lipids and one or more PEG-modified lipids. In an aspect, the composition is a lipid nanoparticle, optionally a liposome.

In an aspect, the compositions comprising the cationic lipids of the present invention may be used in therapy.

### BRIEF DESCRIPTION OF DRAWINGS

FIG. 1 depicts Scheme 1, the reaction scheme for Example 1.

FIG. 2 depicts Scheme 2, the reaction scheme for Example 2.

FIG. 3 depicts Scheme 3, the reaction scheme for Example 3.

FIG. 4 depicts Scheme 4, the reaction scheme for Example 4.

FIG. 5 depicts Scheme 5, the reaction scheme for Example 5.

FIG. 6 depicts Scheme 6, the reaction scheme for Example 6.

FIG. 7 depicts Scheme 7, the reaction scheme for Example 7.

FIG. 8 depicts Scheme 8, the reaction scheme for Example 8.

FIG. 9 depicts Scheme 9, the reaction scheme for Example 9.

FIG. 10 depicts Scheme 10, the reaction scheme for Example 10.

FIG. 11 depicts Scheme 11, the reaction scheme for Example 11.

FIG. 12 depicts Scheme 12, the reaction scheme for Example 12.

FIG. 13 depicts Scheme 13, the reaction scheme for Example 13.

FIG. 14 depicts Scheme 14, the reaction scheme for Example 14.

FIG. 15 depicts Scheme 15, the reaction scheme for Example 15.

FIG. 16 depicts Scheme 16, the reaction scheme for Example 16.

FIG. 17 depicts Scheme 17, the reaction scheme for Example 17.

FIG. 18 depicts Scheme 18, the reaction scheme for Example 18.

FIG. 19 depicts Scheme 19, the reaction scheme for Example 19.

FIG. 20 depicts Scheme 20, the reaction scheme for Example 20.

FIG. 21 depicts Scheme 21, the reaction scheme for Example 21.

FIG. 22 depicts Scheme 22, the reaction scheme for Example 22.

FIG. 23 depicts Scheme 23, the reaction scheme for Example 23.

FIG. 24 depicts Scheme 24, the reaction scheme for Example 24.

FIG. 25 depicts Scheme 25, the reaction scheme for Example 25.

FIG. 26 depicts Scheme 26, the reaction scheme for Example 26.

FIG. 27 depicts Scheme 27, the reaction scheme for Example 27.

FIG. 28 depicts Scheme 28, the reaction scheme for Example 28.

FIG. 29 depicts Scheme 29, the reaction scheme for Example 29.

FIG. 30 depicts Scheme 30, the reaction scheme for Example 30.

FIG. 31 depicts Scheme 31, the reaction scheme for Example 31.

FIG. 32 depicts Scheme 32, the reaction scheme for Example 32.

FIG. 33 depicts Scheme 33, the reaction scheme for Example 33.

FIG. 34 depicts Scheme 34, the reaction scheme for Example 34.

FIG. 35 depicts Scheme 35, the reaction scheme for Example 35.

FIG. 36 depicts Scheme 36, the reaction scheme for Example 36.

FIG. 37 depicts Scheme 37, the reaction scheme for Example 37.

FIG. 38 depicts Scheme 38, the reaction scheme for Example 38.

FIG. 39 depicts Scheme 39, the reaction scheme for Example 39.

FIG. 40 depicts Scheme 40, the reaction scheme for Example 40.

FIG. 41 depicts Scheme 41, the reaction scheme for Example 41.

FIG. 42 depicts Scheme 42, the reaction scheme for Example 42.

FIG. 43 depicts Scheme 43, the reaction scheme for Example 43.

FIG. 44 depicts Scheme 44, the reaction scheme for Example 44.

FIG. 45 depicts Scheme 45, the reaction scheme for Example 45.

FIG. 46 depicts Scheme 46, the reaction scheme for Example 46.

FIG. 47 depicts Scheme 47, the reaction scheme for Example 47.

FIG. 48 depicts Scheme 48, the reaction scheme for Example 48.

FIG. 49 depicts Scheme 49, the reaction scheme for Example 49.

FIG. 50 depicts Scheme 50, the reaction scheme for Example 50.

FIG. 51 depicts in vivo hEPO protein production resulting from the intramuscular delivery of hEPO mRNA using lipid nanoparticles comprising Compounds XII, XIV, XV, XXV, XXXII and XXXVIII as described herein. As shown in this Figure, use of these compounds as part of a lipid nanoparticle can result in high levels of in vivo hEPO protein production after administration.

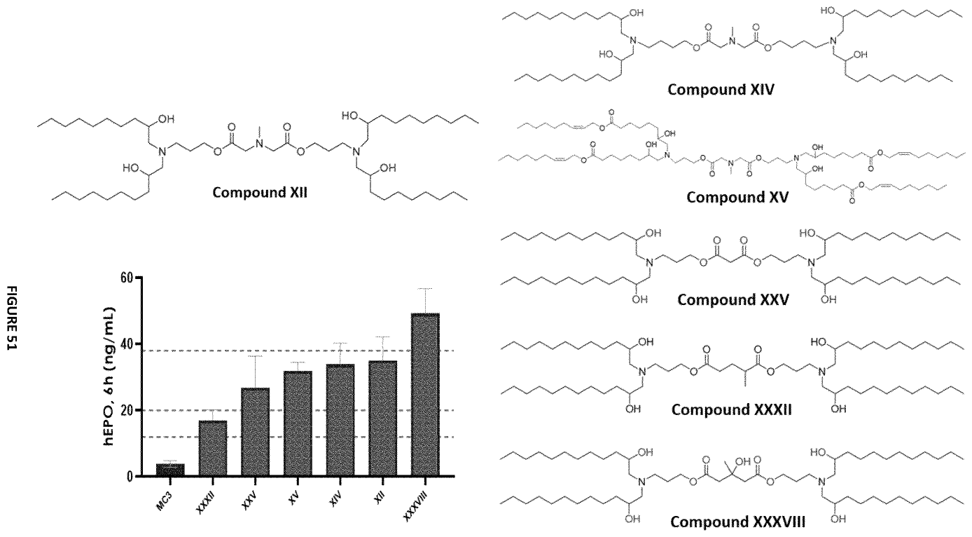

### DETAILED DESCRIPTION OF CERTAIN EMBODIMENTS

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Amino acid: As used herein, the term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an I-amino acid. “Standard amino acid” refers to any of the twenty standard I-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Aromatic Amino Acid or Residue: As used herein, the term “aromatic amino acid or residue” refers to a hydrophilic or hydrophobic amino acid or residue having a side chain that includes at least one aromatic or heteroaromatic ring. Aromatic amino acids or residues include L-amino acids, D-amino acids or racemates. Genetically encoded aromatic amino acids include L-Phe (F), L-Tyr (Y), L-His (H) and L-Trp (W). Although owing to the pKa of its heteroaromatic nitrogen atom L-His (H) is sometimes classified as a basic residue, herein histidine is classified as an aromatic residue as its side chain includes a heteroaromatic ring. Examples of aromatic amino acids include the following:

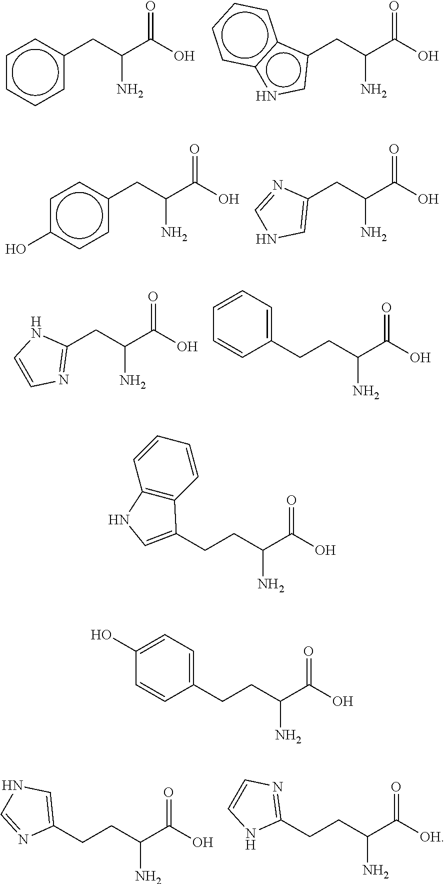

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the term “biologically active” refers to a characteristic of any agent that has activity in a biological system, and particularly in an organism. For instance, an agent that, when administered to an organism, has a biological effect on that organism, is considered to be biologically active.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery”).

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalents thereof, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Helper lipid: The term “helper lipid” as used herein refers to any neutral or zwitterionic lipid material including cholesterol. Without wishing to be held to a particular theory, helper lipids may add stability, rigidity, and/or fluidity within lipid bilayers/nanoparticles.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Liposome: As used herein, the term “liposome” refers to any lamellar, multilamellar, or solid nanoparticle vesicle. Typically, a liposome as used herein can be formed by mixing one or more lipids or by mixing one or more lipids and polymer(s). In some embodiments, a liposome suitable for the present invention contains a cationic lipid(s) and optionally non-cationic lipid(s), optionally cholesterol-based lipid(s), and/or optionally PEG-modified lipid(s).

messenger RNA (mRNA): As used herein, the term “messenger RNA (mRNA)” or “mRNA” refers to a polynucleotide that encodes at least one polypeptide. mRNA as used herein encompasses both modified and unmodified RNA. The term “modified mRNA” related to mRNA comprising at least one chemically modified nucleotide. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C5 propynyl-cytidine, C5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. In some embodiments, “nucleic acid” encompasses ribonucleic acids (RNA), including but not limited to any one or more of interference RNAs (RNAi), small interfering RNA (siRNA), short hairpin RNA (shRNA), antisense RNA (aRNA), messenger RNA (mRNA), modified messenger RNA (mmRNA), long non-coding RNA (lncRNA), micro-RNA (miRNA) multimeric coding nucleic acid (MCNA), polymeric coding nucleic acid (PCNA), guide RNA (gRNA) and CRISPR RNA (crRNA). In some embodiments, “nucleic acid” encompasses deoxyribonucleic acid (DNA), including but not limited to any one or more of single-stranded DNA (ssDNA), double-stranded DNA (dsDNA) and complementary DNA (cDNA). In some embodiments, “nucleic acid” encompasses both RNA and DNA. In embodiments, DNA may be in the form of antisense DNA, plasmid DNA, parts of a plasmid DNA, pre-condensed DNA, a product of a polymerase chain reaction (PCR), vectors (e.g., P1, PAC, BAC, YAC, artificial chromosomes), expression cassettes, chimeric sequences, chromosomal DNA, or derivatives of these groups. In embodiments, RNA may be in the form of messenger RNA (mRNA), ribosomal RNA (rRNA), signal recognition particle RNA (7 SL RNA or SRP RNA), transfer RNA (tRNA), transfer-messenger RNA (tmRNA), small nuclear RNA (snRNA), small nucleolar RNA (snoRNA), SmY RNA, small Cajal body-specific RNA (scaRNA), guide RNA (gRNA), ribonuclease P (RNase P), Y RNA, telomerase RNA component (TERC), spliced leader RNA (SL RNA), antisense RNA (aRNA or asRNA), cis-natural antisense transcript (cis-NAT), CRISPR RNA (crRNA), long noncoding RNA (lncRNA), micro-RNA (miRNA), piwi-interacting RNA (piRNA), small interfering RNA (siRNA), transacting siRNA (tasiRNA), repeat associated siRNA (rasiRNA), 73K RNA, retrotransposons, a viral genome, a viroid, satellite RNA, or derivatives of these groups. In some embodiments, a nucleic acid is a mRNA encoding a protein such as an enzyme.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, non-toxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quaternization of an amine using an appropriate electrophile, e.g., an alkyl halide, to form a quarternized alkylated amino salt.

Systemic distribution or delivery: As used herein, the terms “systemic distribution” or “systemic delivery,” or grammatical equivalents thereof, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### Chemical Definitions

Acyl: As used herein, the term “acyl” refers to R^{Z}—(C=O)—, wherein R^{Z} is, for example, any alkyl, alkenyl, alkynyl, heteroalkyl or heteroalkylene.

Aliphatic: As used herein, the term aliphatic refers to C₁-C₅₀ hydrocarbons and includes both saturated and unsaturated hydrocarbons. An aliphatic may be linear, branched, or cyclic. For example, C₁-C₂₀ aliphatics can include C₁-C₂₀ alkyls (e.g., linear or branched C₁-C₂₀ saturated alkyls), C₂-C₂₀ alkenyls (e.g., linear or branched C₄-C₂₀ dienyls, linear or branched C₆-C₂₀ trienyls, and the like), and C₂-C₂₀ alkynyls (e.g., linear or branched C₂-C₂₀ alkynyls). C₁-C₂₀ aliphatics can include C₃-C₂₀ cyclic aliphatics (e.g., C₃-C₂₀ cycloalkyls, C₄-C₂₀ cycloalkenyls, or C₈-C₂₀ cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR″, —CO₂H, —CO₂R″, —CN, —OH, —OR″, —OCOR′, —OCO₂R″, —NH₂, —NHR″, —N(R″)₂, —SR″ or —SO₂R″, wherein each instance of R″ independently is C₁-C₂₀ aliphatic (e.g., C₁-C₂₀ alkyl, C₁-C₁₅ alkyl, C₁-C₁₀ alkyl, or C₁-C₃ alkyl).

In embodiments, R″ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R″ independently is unsubstituted C₁-C₃ alkyl. In embodiments, the aliphatic is unsubstituted. In embodiments, the aliphatic does not include any heteroatoms. Alkyl: As used herein, the term “alkyl” means acyclic linear and branched hydrocarbon groups, e.g. “C₁-C₃₀ alkyl” refers to alkyl groups having 1-30 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR″, —CO₂H, —CO₂R″, —CN, —OH, —OR″, —OCOR′, —OCO₂R″, —NH₂, —NHR″, —N(R″)₂, —SR″ or —SO₂R″, wherein each instance of R″ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R″ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R″ independently is unsubstituted C₁-C₃ alkyl. In embodiments, the alkyl is substituted (e.g., with 1, 2, 3, 4, 5, or 6 substituent groups as described herein). In embodiments, an alkyl group is substituted with a-OH group and may also be referred to herein as a “hydroxyalkyl” group, where the prefix denotes the —OH group and “alkyl” is as described herein.

As used herein, “alkyl” also refers to a radical of a straight-chain or branched saturated hydrocarbon group having from 1 to 50 carbon atoms (“C₁-C₅₀ alkyl”). In some embodiments, an alkyl group has 1 to 40 carbon atoms (“C₁-C₄₀ alkyl”). In some embodiments, an alkyl group has 1 to 30 carbon atoms (“C₁-C₃₀ alkyl”). In some embodiments, an alkyl group has 1 to 20 carbon atoms (“C₁-C₂₀ alkyl”). In some embodiments, an alkyl group has 1 to 10 carbon atoms (“C₁-C₁₀ alkyl”). In some embodiments, an alkyl group has 1 to 9 carbon atoms (“C₁-C₉ alkyl”). In some embodiments, an alkyl group has 1 to 8 carbon atoms (“C₁-C₈ alkyl”). In some embodiments, an alkyl group has 1 to 7 carbon atoms (“C₁-C₇ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”).

In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆ alkyl groups include, without limitation, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁-C₅₀ alkyl. In certain embodiments, the alkyl group is a substituted C₁-C₅₀ alkyl.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R″ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R″ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms. Alkenyl: As used herein, “alkenyl” means any linear or branched hydrocarbon chains having one or more unsaturated carbon-carbon double bonds that may occur in any stable point along the chain, e.g. “C₂-C₃₀ alkenyl” refers to an alkenyl group having 2-30 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In embodiments, the alkenyl comprises 1, 2, or 3 carbon-carbon double bond. In embodiments, the alkenyl comprises a single carbon-carbon double bond. In embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R″ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R″ independently is unsubstituted C₁-C₃ alkyl. In embodiments, the alkenyl is unsubstituted. In embodiments, the alkenyl is substituted (e.g., with 1, 2, 3, 4, 5, or 6 substituent groups as described herein). In embodiments, an alkenyl group is substituted with a-OH group and may also be referred to herein as a “hydroxyalkenyl” group, where the prefix denotes the —OH group and “alkenyl” is as described herein.

As used herein, “alkenyl” also refers to a radical of a straight-chain or branched hydrocarbon group having from 2 to 50 carbon atoms and one or more carbon-carbon double bonds (e.g., 1, 2, 3, or 4 double bonds) (“C₂-C₅₀ alkenyl”). In some embodiments, an alkenyl group has 2 to 40 carbon atoms (“C₂-C₄₀ alkenyl”). In some embodiments, an alkenyl group has 2 to 30 carbon atoms (“C₂-C₃₀ alkenyl”). In some embodiments, an alkenyl group has 2 to 20 carbon atoms (“C₂-C₂₀ alkenyl”). In some embodiments, an alkenyl group has 2 to 10 carbon atoms (“C₂-C₁₀ alkenyl”). In some embodiments, an alkenyl group has 2 to 9 carbon atoms (“C₂-C₉ alkenyl”). In some embodiments, an alkenyl group has 2 to 8 carbon atoms (“C₂-C₈ alkenyl”). In some embodiments, an alkenyl group has 2 to 7 carbon atoms (“C₂-C₇ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include, without limitation, ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-C₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂-C₅₀ alkenyl. In certain embodiments, the alkenyl group is a substituted C₂-C₅₀ alkenyl.

Alkynyl: As used herein, “alkynyl” means any hydrocarbon chain of either linear or branched configuration, having one or more carbon-carbon triple bonds occurring in any stable point along the chain, e.g., “C₂-C₃₀ alkynyl”, refers to an alkynyl group having 2-30 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R″ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R″ independently is unsubstituted C₁-C₃ alkyl. In embodiments, the alkynyl is unsubstituted. In embodiments, the alkynyl is substituted (e.g., with 1, 2, 3, 4, 5, or 6 substituent groups as described herein).

As used herein, “alkynyl” also refers to a radical of a straight-chain or branched hydrocarbon group having from 2 to 50 carbon atoms and one or more carbon-carbon triple bonds (e.g., 1, 2, 3, or 4 triple bonds) and optionally one or more double bonds (e.g., 1, 2, 3, or 4 double bonds) (“C₂-C₅₀ alkynyl”). An alkynyl group that has one or more triple bonds, and one or more double bonds is also referred to as an “ene-yne”. In some embodiments, an alkynyl group has 2 to 40 carbon atoms (“C₂-C₄₀ alkynyl”). In some embodiments, an alkynyl group has 2 to 30 carbon atoms (“C₂-C₃₀ alkynyl”). In some embodiments, an alkynyl group has 2 to 20 carbon atoms (“C₂-C₂₀ alkynyl”). In some embodiments, an alkynyl group has 2 to 10 carbon atoms (“C₂-C₁₀ alkynyl”). In some embodiments, an alkynyl group has 2 to 9 carbon atoms (“C₂-C₉ alkynyl”). In some embodiments, an alkynyl group has 2 to 8 carbon atoms (“C₂-C₈ alkynyl”). In some embodiments, an alkynyl group has 2 to 7 carbon atoms (“C₂-C₇ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include, without limitation, ethynyl (C₂), 1-propynyl (C₃), 2-propynyl (C₃), 1-butynyl (C₄), 2-butynyl (C₄), and the like. Examples of C₂-C₆ alkenyl groups include the aforementioned C₂-C₄ alkynyl groups as well as pentynyl (C₅), hexynyl (C₆), and the like. Additional examples of alkynyl include heptynyl (C₇), octynyl (C), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂-C₅₀ alkynyl. In certain embodiments, the alkynyl group is a substituted C₂-C₅₀ alkynyl.

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In embodiments, an aryl group has 6 ring carbon atoms (“C₆ aryl,” e.g., phenyl). In some embodiments, an aryl group has 10 ring carbon atoms (“C₁₀ aryl,” e.g., naphthyl such as 1-naphthyl and 2-naphthyl). In some embodiments, an aryl group has 14 ring carbon atoms (“C₁₄ aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

As used herein, “aryl” also refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆-C₁₄ aryl. In certain embodiments, the aryl group is a substituted C₆-C₁₄ aryl.

Arylene: The term “arylene” as used herein refers to an aryl group that is divalent (that is, having two points of attachment to the molecule). Exemplary arylenes include phenylene (e.g., unsubstituted phenylene or substituted phenylene).

Carbocyclyl: As used herein, “carbocyclyl” or “carbocyclic” refers to a radical of a non-aromatic cyclic hydrocarbon group having from 3 to 10 ring carbon atoms (“C₃-C₁₀ carbocyclyl”) and zero heteroatoms in the non-aromatic ring system. In some embodiments, a carbocyclyl group has 3 to 8 ring carbon atoms (“C₃-C₈ carbocyclyl”). In some embodiments, a carbocyclyl group has 3 to 7 ring carbon atoms (“C₃-C₇ carbocyclyl”). In some embodiments, a carbocyclyl group has 3 to 6 ring carbon atoms (“C₃-C₆ carbocyclyl”). In some embodiments, a carbocyclyl group has 4 to 6 ring carbon atoms (“C₄-C₆ carbocyclyl”). In some embodiments, a carbocyclyl group has 5 to 6 ring carbon atoms (“C₅-C₆ carbocyclyl”). In some embodiments, a carbocyclyl group has 5 to 10 ring carbon atoms (“C₅-C₁₀ carbocyclyl”). Exemplary C₃-C₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ carbocyclyl groups include, without limitation, the aforementioned C₃-C₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃-C₁₀ carbocyclyl groups include, without limitation, the aforementioned C₃-C₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃-C₁₀ carbocyclyl. In certain embodiments, the carbocyclyl group is a substituted C₃-C₁₀ carbocyclyl.

In some embodiments, “carbocyclyl” or “carbocyclic” is referred to as a “cycloalkyl”, i.e., a monocyclic, saturated carbocyclyl group having from 3 to 10 ring carbon atoms (“C₃-C₁₀ cycloalkyl”). In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆, cycloalkyl”). In some embodiments, a cycloalkyl group has 4 to 6 ring carbon atoms (“C₄-C₆ cycloalkyl”). In some embodiments, a cycloalkyl group has 5 to 6 ring carbon atoms (“C₅-C₆ cycloalkyl”). In some embodiments, a cycloalkyl group has 5 to 10 ring carbon atoms (“C₅-C₁₀ cycloalkyl”). Examples of C₅-C₆ cycloalkyl groups include cyclopentyl (C₅) and cyclohexyl (C₅). Examples of C₃-C₆ cycloalkyl groups include the aforementioned C₅-C₆ cycloalkyl groups as well as cyclopropyl (C₃) and cyclobutyl (C₄). Examples of C₃-C₈ cycloalkyl groups include the aforementioned C₃-C₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃-C₁₀ cycloalkyl. In certain embodiments, the cycloalkyl group is a substituted C₃-C₁₀ cycloalkyl.

Halogen: As used herein, the term “halogen” means fluorine, chlorine, bromine, or iodine.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

Heteroalkylene: The term “heteroalkylene,” as used herein, represents a divalent form of a heteroalkyl group as described herein.

Heteroaryl: The term “heteroaryl,” as used herein, is fully unsaturated heteroatom-containing ring wherein at least one ring atom is a heteroatom such as, but not limited to, nitrogen and oxygen.

As used herein, “heteroaryl” also refers to a radical of a 5-14 membered monocyclic or polycyclic (e.g., bicyclic or tricyclic) 4n+2 aromatic ring system (e.g., having 6, 10, or 14 π electrons shared in a cyclic array) having ring carbon atoms and 1 or more (e.g., 1, 2, 3, or 4 ring heteroatoms) ring heteroatoms provided in the aromatic ring system, wherein each heteroatom is independently selected from oxygen, sulfur, nitrogen, boron, silicon, and phosphorus (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-10 membered heteroaryl”). In some embodiments, a heteroaryl group is a 5-8 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-8 membered heteroaryl”). In some embodiments, a heteroaryl group is a 5-6 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-6 membered heteroaryl”). In some embodiments, the 5-6 membered heteroaryl has 1 or more (e.g., 1, 2, or 3) ring heteroatoms selected from oxygen, sulfur, nitrogen, boron, silicon, and phosphorus. In some embodiments, the 5-6 membered heteroaryl has 1 or 2 ring heteroatoms selected from oxygen, sulfur, nitrogen, boron, silicon, and phosphorus. In some embodiments, the 5-6 membered heteroaryl has 1 ring heteroatom selected from oxygen, sulfur, nitrogen, boron, silicon, and phosphorus. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, “heterocyclyl” or “heterocyclic” refers to a radical of a 3- to 14-membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)) and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-10 membered heterocyclyl”). In some embodiments, a heterocyclyl group is a 5-8 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-8 membered heterocyclyl”). In some embodiments, a heterocyclyl group is a 5-6 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-6 membered heterocyclyl”). In some embodiments, the 5-6 membered heterocyclyl has 1 or more (e.g., 1, 2, or 3) ring heteroatoms selected from oxygen, sulfur, nitrogen, boron, silicon, and phosphorus. In some embodiments, the 5-6 membered heterocyclyl has 1 or 2 ring heteroatoms selected from oxygen, sulfur, nitrogen, boron, silicon, and phosphorus. In some embodiments, the 5-6 membered heterocyclyl has 1 ring heteroatom selected from oxygen, sulfur, nitrogen, boron, silicon, and phosphorus.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation. tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. The heterocycloalkyl group can be substituted or unsubstituted.

As understood from the above, alkyl, alkenyl, alkynyl, acyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂, —SO₃H, —OH, —OR^{aa}, —ON(R^{bb})₂, —N(R^{bb})₂, —N(R^{bb})₃+X⁻, —N(OR^{cc})R^{bb}, —SeH, —SeR^{aa}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃—OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁-C₅₀ alkyl, C₂-C₅₀ alkenyl, C₂-C₅₀ alkynyl, C₃-C₁₄ carbocyclyl, 3-14 membered heterocyclyl, C₆-C₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, =NNR^{bb}C(═O)R^{aa}, =NNR^{bb}C(═O)OR^{aa}, =NNR^{bb}S(═O)₂R^{aa}, ═NR^{bb}, or =NOR^{cc};
each instance of R^{aa} is, independently, selected from C₁-C₅₀ alkyl, C₂-C₅₀ alkenyl, C₂-C₅₀ alkynyl, C₃-C₁₀ carbocyclyl, 3-14 membered heterocyclyl, C₆-C₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —C₀O₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁-C₅₀ alkyl, C₂-C₅₀ alkenyl, C₂-C₅₀ alkynyl, C₃-C₁₀ carbocyclyl, 3-14 membered heterocyclyl, C₆-C₁₄ aryl, and 5-14 membered heteroaryl, or two R^{bb} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁-C₅₀ alkyl, C₂-C₅₀ alkenyl, C₂-C₅₀ alkynyl, C₃-C₁₀ carbocyclyl, 3-14 membered heterocyclyl, C₆-C₁₄ aryl, and 5-14 membered heteroaryl, or two R^{cc} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁-C₅₀ alkyl, C₂-C₅₀ alkenyl, C₂-C₅₀ alkynyl, C₃-C₁₀ carbocyclyl, 3-10 membered heterocyclyl, C₆-C₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{g9} groups, or two geminal R^{dd} substituents can be joined to form ═O or =S;
each instance of R^{ee} is, independently, selected from C₁-C₅₀ alkyl, C₂-C₅₀ alkenyl, C₂-C₅₀ alkynyl, C₃-C₁₀ carbocyclyl, C₆-C₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{gg} is, independently, selected from hydrogen, C₁-C₅₀ alkyl, C₂-C₅₀ alkenyl, C₂-C₅₀ alkynyl, C₃-C₁₀ carbocyclyl, 3-10 membered heterocyclyl, C₆-C₁₀ aryl and 5-10 membered heteroaryl, or two R^{ff} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{g9} groups; and
each instance of R^{gg} is, independently, halogen, —CN, —NO₂, —N₃, —SO₂H, —SO₃H, —OH, —OC₁-C₅₀ alkyl, —ON(C₁-C₅₀ alkyl)₂, —N(C₁-C₅₀ alkyl)₂, —N(C₁-C₅₀ alkyl)₃+X⁻, —NH(C₁-C₅₀ alkyl)₂+X⁻, —NH₂(C₁-C₅₀ alkyl)+X⁻, —NH₃+X⁻, —N(OC₁-C₅₀ alkyl)(C₁-C₅₀ alkyl), —N(OH)(C₁-C₅₀ alkyl), —NH(OH), —SH, —SC₁-C₅₀ alkyl, —SS(C₁-C₅₀ alkyl), —C(═O)(C₁-C₅₀ alkyl), —CO₂H, —CO₂(C₁-C₅₀ alkyl), —OC(═O)(C₁-C₅₀ alkyl), —OCO₂(C₁-C₅₀ alkyl), —C(═O)NH₂, —C(═O)N(C₁-C₅₀ alkyl)₂, —OC(═O)NH(C₁-C₅₀ alkyl), —NHC(═O)(C₁-C₅₀ alkyl), —N(C₁-C₅₀ alkyl)C(═O)(C₁-C₅₀ alkyl), —NHCO₂(C₁-C₅₀ alkyl), —NHC(═O)N(C₁-C₅₀ alkyl)₂, —NHC(═O)NH(C₁-C₅₀ alkyl), —NHC(═O)NH₂, —C(═NH)O(C₁-C₅₀ alkyl), —OC(═NH)(C₁-C₅₀ alkyl), —OC(═NH)OC₁-C₅₀ alkyl, —C(═NH)N(C₁-C₅₀ alkyl)₂, —C(═NH)NH(C₁-C₅₀ alkyl), —C(═NH)NH₂, —OC(═NH)N(C₁-C₅₀ alkyl)₂, —OC(NH)NH(C₁-C₅₀ alkyl), —OC(NH)NH₂, —NHC(NH)N(C₁-C₅₀ alkyl)₂, —NHC(═NH)NH₂, —NHSO₂(C₁-C₅₀ alkyl), —SO₂N(C₁-C₅₀ alkyl)₂, —SO₂NH(C₁-C₅₀ alkyl), —SO₂NH₂, —SO₂(C₁-C₅₀ alkyl), —SO₂O(C₁-C₅₀ alkyl), —OSO₂(C₁-C₆ alkyl), —SO(C₁-C₆ alkyl), —Si(C₁-C₅₀ alkyl)₃, —OSi(C₁-C₆ alkyl)₃, —C(═S)N(C₁-C₅₀ alkyl)₂, C(═S)NH(C₁-C₅₀ alkyl), C(═S)NH₂, —C(═O)S(C₁-C₆ alkyl), —C(═S)S(C₁-C₆ alkyl), —SC(═S)S(C₁-C₆ alkyl), —P(═O)₂(C₁-C₅₀ alkyl), —P(═O)(C₁-C₅₀ alkyl)₂, —OP(═O)(C₁-C₅₀ alkyl)₂, —OP(═O)(OC₁-C₅₀ alkyl)₂, C₁-C₅₀ alkyl, C₂-C₅₀ alkenyl, C₂-C₅₀ alkynyl, C₃-C₁₀ carbocyclyl, C₆-C₁₀ aryl, 3-10 membered heterocyclyl, 5-10 membered heteroaryl; or two geminal R^{gg} substituents can be joined to form ═O or =S; wherein X⁻ is a counterion.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁-C₅₀ alkyl, C₂-C₅₀ alkenyl, C₂-C₅₀ alkynyl, C₃-C₁₀ carbocyclyl, 3-14 membered heterocyclyl, C₆-C₁₄ aryl, and 5-14 membered heteroaryl, or two R^{cc} groups, together with the N atom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (lpaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary sulfur protecting groups include, but are not limited to, alkyl, benzyl, p-methoxybenzyl, 2,4,6-trimethylbenzyl, 2,4,6-trimethoxybenzyl, o-hydroxybenzyl, p-hydroxybenzyl, o-acetoxybenzyl, p-acetoxybenzyl, p-nitrobenzyl, 4-picolyl, 2-quinolinylmethyl, 2-picolyl N-oxido, 9-anthrylmethyl, 9-fluorenylmethyl, xanthenyl, ferrocenylmethyl, diphenylmethyl, bis(4-methoxyphenyl)methyl, 5-dibenzosuberyl, triphenylmethyl, diphenyl-4-pyridylmethyl, phenyl, 2,4-dinitrophenyl, t-butyl, 1-adamantyl, methoxymethyl (MOM), isobutoxymethyl, benzyloxymethyl, 2-tetrahydropyranyl, benzylthiomethyl, phenylthiomethyl, thiazolidino, acetamidomethyl, trimethylacetamidomethyl, benzamidomethyl, allyloxycarbonylaminomethyl, phenylacetamidomethyl, phthalimidomethyl, acetylmethyl, carboxymethyl, cyanomethyl, (2-nitro-1-phenyl)ethyl, 2-(2,4-dinitrophenyl)ethyl, 2-cyanoethyl, 2-(Trimethylsilyl)ethyl, 2,2-bis(carboethoxy)ethyl, (1-m-nitrophenyl-2-benzoyl)othyl, 2-phenylsulfonylethyl, 2-(4-methylphenylsulfonyl)-2-methylprop-2-yl, acetyl, benzoyl, trifluoroacetyl, N-[[(p-biphenylyl)isopropoxy]carbonyl]-N-methyl]-γ-aminothiobutyrate, 2,2,2-trichloroethoxycarbonyl, t-butoxycarbonyl, benzyloxycarbonyl, p-methoxybenzyloxycarbonyl, N-ethyl, N-methoxymethyl, sulfonate, sulfenylthiocarbonate, 3-nitro-2-pyridinesulfenyl sulfide, oxathiolone.

### Compounds of the Invention

Liposomal-based vehicles are considered an attractive carrier for therapeutic agents and remain subject to continued development efforts. While liposomal-based vehicles that comprise certain lipid components have shown promising results with regard to encapsulation, stability and site localization, there remains a great need for improvement of liposomal-based delivery systems. For example, a significant drawback of liposomal delivery systems relates to the construction of liposomes that have sufficient cell culture or in vivo stability to reach desired target cells and/or intracellular compartments, and the ability of such liposomal delivery systems to efficiently release their encapsulated materials to such target cells.

In particular, there remains a need for improved lipid compounds that demonstrate improved pharmacokinetic properties, and which are capable of delivering macromolecules, such as nucleic acids, to a wide variety of cell types and tissues with enhanced efficiency. Importantly, there also remains a particular need for novel lipid compounds that are characterized as having improved safety profiles and are capable of efficiently delivering encapsulated nucleic acids and polynucleotides to targeted cells, tissues and organs.

Described herein is a novel class of cationic lipid compounds for improved in vivo delivery of therapeutic agents, such as nucleic acids. In particular, a cationic lipid described herein may be used, optionally with other lipids, to formulate a lipid-based nanoparticle (e.g., liposome) for encapsulating therapeutic agents, such as nucleic acids (e.g., DNA, siRNA, mRNA, microRNA) for therapeutic use, such as disease treatment and prevention (vaccine) purposes.

In embodiments, compounds of the invention as described herein can provide one or more desired characteristics or properties. That is, in certain embodiments, compounds of the invention as described herein can be characterized as having one or more properties that afford such compounds advantages relative to other similarly classified lipids. For example, compounds disclosed herein can allow for the control and tailoring of the properties of liposomal compositions (e.g., lipid nanoparticles) of which they are a component. In particular, compounds disclosed herein can be characterized by enhanced transfection efficiencies and their ability to provoke specific biological outcomes. Such outcomes can include, for example enhanced cellular uptake, endosomal/lysosomal disruption capabilities and/or promoting the release of encapsulated materials (e.g., polynucleotides) intracellularly. Additionally, the compounds disclosed herein have advantageous pharmacokinetic properties, biodistribution, and efficiency.

The present application demonstrates that the cationic lipids of the present invention are synthetically tractable from readily available starting materials.

Additionally, the cationic lipids of the present invention have cleavable groups such as ester groups, amide groups and disulphides. These cleavable groups (e.g. esters, amides and disulphides) are contemplated to improve biodegradability and thus contribute to the lipids' favorable safety profiles.

Provided herein are compounds which are cationic lipids. For example, the cationic lipids of the present invention include compounds having a structure according to Formula (I):

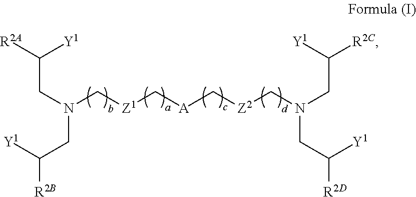

or a pharmaceutically acceptable salt thereof,
wherein A is selected from —N(R¹)— or —S—S—;
R¹ is optionally substituted (C₁-C₆)alkyl;
a and c are integers that are each independently selected from 1, 2, 3 or 4;
b and d are integers that are each independently selected from 1, 2, 3, 4, 5 or 6;
Z¹ is selected from a covalent bond,

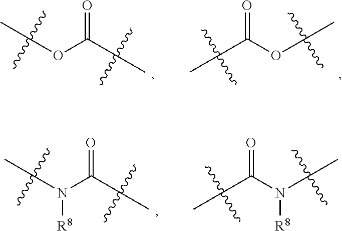

or —S—S—, wherein the left hand side of each depicted structure is bound to the —(CH₂)_{b}—;
Z² is selected from a covalent bond,

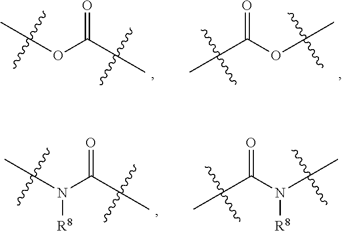

or —S—S—, wherein the right hand side of each depicted structure is bound to the —(CH₂)_{d}—;
each Y¹ is independently selected from hydrogen or —OH;
each R⁸ is independently selected from hydrogen or optionally substituted (C₁-C₆)alkyl;
R^{2A}, R^{2B}, R^{2C}, and R^{2D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y¹)—when W¹ is a covalent bond.

In embodiments, the cationic lipids of the present invention include compounds of Formula (I) wherein A is —N(R¹)—. In embodiments, the cationic lipids of the present invention include compounds having a structure according to Formula (IA):

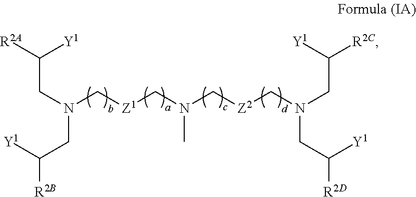

or a pharmaceutically acceptable salt thereof.

In embodiments, the cationic lipids of the present invention include compounds of Formula (I) wherein A is —N(R¹)— and Y¹ is OH. In embodiments, the cationic lipids of the present invention include compounds having a structure according to Formula (IA1):

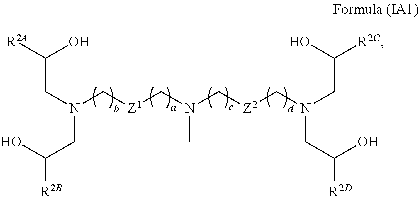

or a pharmaceutically acceptable salt thereof.

In embodiments, the cationic lipids of the present invention include compounds of Formula (I) wherein wherein A is —N(R¹)—, Y¹ is OH, and Z¹ and Z² are each an ester. In embodiments, the cationic lipids of the present invention include compounds of Formula (I)
wherein wherein A is —N(R¹)—, Y¹ is OH, Z¹ is

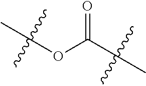 [SMILES: *OC(*)=O]

wherein the left hand side of the depicted structure is bound to the —(CH₂)_{b}—, and Z² is

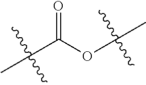 [SMILES: *OC(*)=O]

wherein the right hand side of the depicted structure is bound to the —(CH₂)_{d}—. In embodiments, the cationic lipids of the present invention include compounds having a structure according to Formula (IA1i):

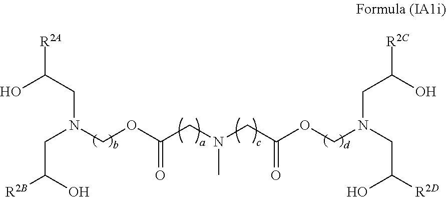

or a pharmaceutically acceptable salt thereof.

In embodiments, the cationic lipids of the present invention include compounds of Formula (I) wherein wherein A is —N(R¹)—, Y¹ is OH, a=1, c=1, and Z¹ and Z² are each an ester. In embodiments, the cationic lipids of the present invention include compounds of Formula (I) wherein A is —N(R¹)—, Y¹ is OH, a=1, c=1, Z¹ is

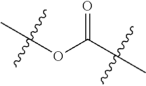 [SMILES: *OC(*)=O]

wherein the left hand side of the depicted structure is bound to the —(CH₂)_{b}—, and Z² is

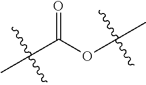 [SMILES: *OC(*)=O]

wherein the right hand side of the depicted structure is bound to the —(CH₂)_{d}—. In embodiments, the cationic lipids of the present invention include compounds having a structure according to Formula (IA1ia):

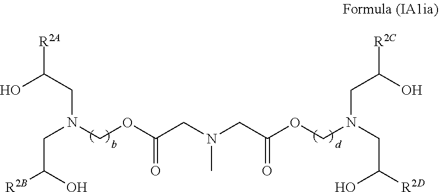

or a pharmaceutically acceptable salt thereof.

In embodiments, the cationic lipids of the present invention include compounds of Formula (I) wherein A=—N(R¹)— and Y¹═H. In embodiments, the cationic lipids of the present invention include compounds having a structure according to Formula (IA2):

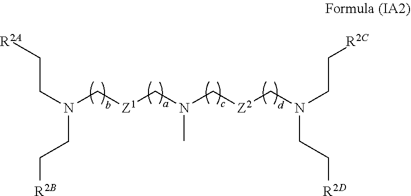

or a pharmaceutically acceptable salt thereof.

In embodiments, the cationic lipids of the present invention include compounds having a structure according to Formula (IB):

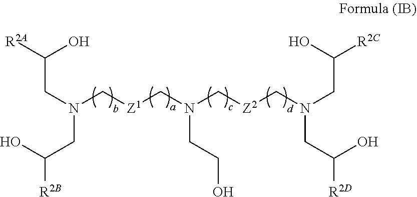

or a pharmaceutically acceptable salt thereof.

In embodiments, the cationic lipids of the present invention include compounds having a structure according to Formula (IC):

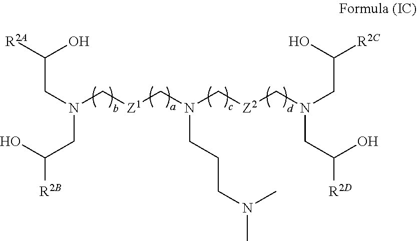

or a pharmaceutically acceptable salt thereof.

In embodiments, the cationic lipids of the present invention include compounds having a structure according to Formula (ID):

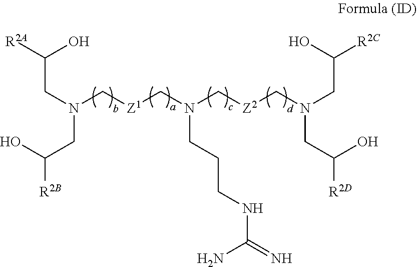

or a pharmaceutically acceptable salt thereof.

In embodiments, the cationic lipids of the present invention include compounds having a structure according to Formula (IE):

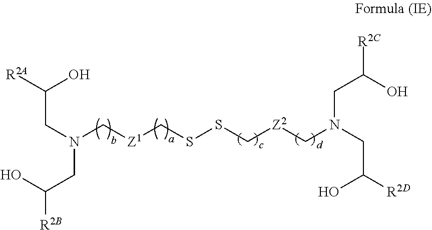

or a pharmaceutically acceptable salt thereof.

In embodiments, the cationic lipids of the present invention include compounds having a structure according to Formula (IA1ii):

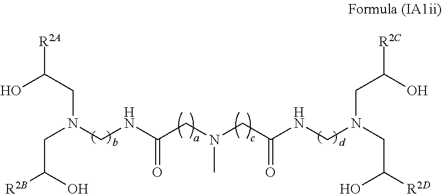

or a pharmaceutically acceptable salt thereof.

In embodiments, the cationic lipids of the present invention include compounds having a structure according to Formula (IA1iia):

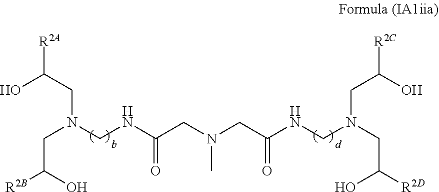

or a pharmaceutically acceptable salt thereof.

In embodiments, the cationic lipids of the present invention include compounds having a structure according to Formula (IA1iii):

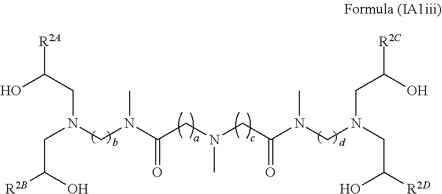

or a pharmaceutically acceptable salt thereof.

In embodiments, the cationic lipids of the present invention include compounds having a structure according to Formula (IA1iiia):

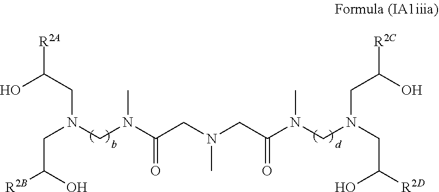

or a pharmaceutically acceptable salt thereof.

In embodiments, A is —N(R¹)—. In embodiments, R¹ is (C₁-C₆)alkyl. In embodiments, R¹ is methyl. In embodiments, R¹ is (C₁-C₆)alkylene-R^{A}, wherein R^{A} is selected from —OH, —N(R⁶)(R⁷), or

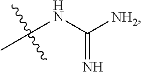

wherein each R⁶ and R⁷ is independently selected from optionally substituted (C₁-C₆)alkyl. In embodiments, R^{A} is —OH. In embodiments, R^{A} is —N(R⁶)(R⁷). In embodiments, R^{A} is

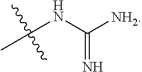 [SMILES: *NC(=N)N]

In embodiments, R⁶ and R⁷ are methyl. In embodiments, A is —S—S—.

In embodiments, a is 1 or 2, preferably wherein the cationic lipid has a structure according to any one of Formula (IA), Formula (IA1), or Formula (IA2). In embodiments, a is 1, preferably wherein the cationic lipid has a structure according to (i) Formula (IB) or (ii) Formula (IA1ii) or Formula (IA1iii). In embodiments, a is 2, preferably wherein the cationic lipid has a structure according to any one of Formula (IC), Formula (ID) or Formula (IE). In embodiments, a is 3. In embodiments, a is 4.

In embodiments, b is 2, 3 or 4, preferably wherein the cationic lipid has a structure according to any one of Formula (IA), Formula (IA1), or Formula (IA2). In embodiments, b is 3 or 4, preferably wherein the cationic lipid has a structure according to (i) Formula (IA1ia) or (ii) Formula (IA1ii), Formula (IA1iia), Formula (IA1iii) or Formula (IA1iiia). In embodiments, b is 3, preferably wherein the cationic lipid has a structure according to any one of Formula (IB), Formula (ID), or Formula (IE). In embodiments, b is 4, preferably wherein the cationic lipid has a structure according to Formula (IC). In embodiments, b is 1. In embodiments, b is 2. In embodiments, b is 5. In embodiments, b is 6.

In embodiments, c is 1 or 2, preferably wherein the cationic lipid has a structure 5 according to any one of Formula (IA), Formula (IA1), or Formula (IA2). In embodiments, c is 1, preferably wherein the cationic lipid has a structure according to (i) Formula (IB) or (ii) Formula (IA1ii) or Formula (IA1iii). In embodiments, c is 2, preferably wherein the cationic lipid has a structure according to any one of Formula (IC), Formula (ID) or Formula (IE). In embodiments, c is 3. In embodiments, c is 4.

In embodiments, d is 2, 3 or 4, preferably wherein the cationic lipid has a structure according to any one of Formula (IA), Formula (IA1), or Formula (IA2). In embodiments, d is 3 or 4, preferably wherein the cationic lipid has a structure according to (i) Formula (IA1ia) or (ii) Formula (IA1ii), Formula (IA1iia), Formula (IA1iii) or Formula (IA1iiia). In embodiments, d is 3, preferably wherein the cationic lipid has a structure according to any one of Formula (IB), Formula (ID) or Formula (IE). In embodiments, d is 4, preferably wherein the cationic lipid has a structure according to Formula (IC). In embodiments, d is 1. In embodiments, d is 2. In embodiments, d is 5. In embodiments, d is 6.

In embodiments, Z¹ is a covalent bond. In embodiments, Z¹ is

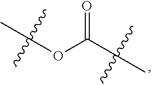 [SMILES: *OC(*)=O]

wherein the left hand side of the depicted structure is bound to the —(CH₂)_{b}—. In embodiments, Z¹ is

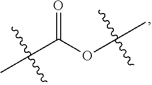 [SMILES: *OC(*)=O]

wherein the left hand side of the depicted structure is bound to the —(CH₂)_{b}—. In embodiments, Z¹ is

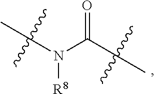

wherein the left hand side of the depicted structure is bound to the —(CH₂)_{b}—. In embodiments, Z¹ is

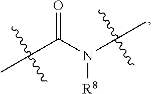

wherein the left hand side of the depicted structure is bound to the —(CH₂)_{b}—. In embodiments, Z¹ is —S—S—.

In embodiments, Z² is a covalent bond. In embodiments, Z² is

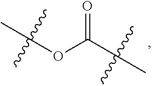 [SMILES: *OC(*)=O]

wherein the right hand side of the depicted structure is bound to the —(CH₂)_{d}—. In embodiments, Z² is

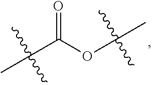 [SMILES: *OC(*)=O]

wherein the right hand side of the depicted structure is bound to the —(CH₂)_{d}—. In embodiments, Z² is

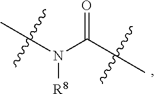

wherein the right hand side of the depicted structure is bound to the —(CH₂)_{d}—. In embodiments, Z² is

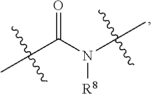

wherein the right hand side of the depicted structure is bound to the —(CH₂)_{d}—. In embodiments, Z² is —S—S—.

In embodiments, Z¹ is

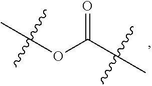 [SMILES: *OC(*)=O]

wherein the left hand side of the depicted structure is bound to the —(CH₂)_{b}—, and Z² is —S—S—, preferably wherein the cationic lipid has a structure according to any one of Formula (IA) or Formula (IA1).

In embodiments, Z¹ and Z² are both —S—S—, preferably wherein the cationic lipid has a structure according to any one of Formula (IA) or Formula (IA1).

In embodiments, Z¹ and Z² are both a covalent bond, preferably wherein the cationic lipid has a structure according to any one of Formula (IA), Formula (IA1) or Formula (IA2).

In embodiments, Z¹ is

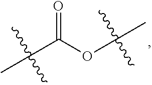 [SMILES: *OC(*)=O]

wherein the left hand side of the depicted structure is bound to the —(CH₂)_{b}—, and Z² is

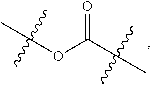 [SMILES: *OC(*)=O]

wherein the right hand side of the depicted structure is bound to the —(CH₂)_{d}—, preferably wherein the cationic lipid has a structure according to any one of Formula (IA), Formula (IA1), Formula (IA2), Formula (IC) or Formula (ID).

In embodiments, Z¹ is

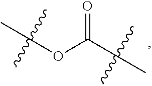 [SMILES: *OC(*)=O]

wherein the left hand side of the depicted structure is bound to the —(CH₂)_{b}—, and Z² is

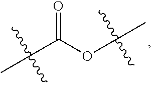 [SMILES: *OC(*)=O]

wherein the right hand side of the depicted structure is bound to the —(CH₂)_{d}—, preferably wherein the cationic lipid has a structure according to any one of Formula (IA2), Formula (IB) or Formula (IE).

In embodiments, Z¹ is

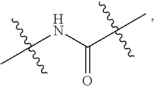 [SMILES: *NC(*)=O]

wherein the left hand side of the depicted structure is bound to the —(CH₂)_{b}—, and Z² is

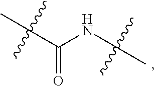 [SMILES: *NC(*)=O]

wherein the right hand side of the depicted structure is bound to the —(CH₂)_{d}—, preferably wherein the cationic lipid has a structure according to any one of Formula (IA) or Formula (IA1).

In embodiments, Z¹ is

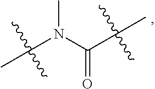 [SMILES: *C(=O)N(*)C]

wherein the left hand side of the depicted structure is bound to the —(CH₂)_{b}—, and Z² is

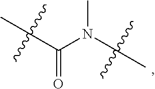 [SMILES: *C(=O)N(*)C]

wherein the right hand side of the depicted structure is bound to the —(CH₂)_{d}—, preferably wherein the cationic lipid has a structure according to any one of Formula (IA) or Formula (IA1).

In embodiments, at least one Y¹ is —OH. In embodiments, at least one Y¹ is hydrogen. In embodiments, Y¹ is —OH. In embodiments, Y¹ is hydrogen.

In embodiments, each R⁸ is hydrogen. In embodiments, each R⁸ is optionally substituted (C₁-C₆)alkyl. In embodiments, each R⁸ is independently selected from hydrogen or methyl. In embodiments, each R⁸ is methyl.

In embodiments, R^{2A} is optionally substituted (C₅-C₂₅)alkyl. In embodiments, R^{2A} is optionally substituted (C₅-C₂₀)alkyl. In embodiments, R^{2A} is optionally substituted (C₅-C₁₅)alkyl. In embodiments, R^{2A} is optionally substituted (C₆-C₁₂)alkyl.

In embodiments, R^{2A} is optionally substituted (C₅-C₂₅)alkenyl. In embodiments, R^{2A} is optionally substituted (C₅-C₂₀)alkenyl. In embodiments, R^{2A} is optionally substituted (C₁₀-C₂₀)alkenyl. In embodiments, R^{2A} is optionally substituted (C₁₅-C₂₀)alkenyl.

In embodiments, R^{2A} is —W¹—X¹. In embodiments, W¹ is a covalent bond. In embodiments, W¹ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W¹ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W⁷ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y¹)—when W¹ is a covalent bond. In embodiments, X¹ is —(*C═O)—O-optionally substituted (C₃-C₂₀)alkyl, wherein the atom marked with a * is connected to W¹ or —CH(Y¹)—when W¹ is a covalent bond. In embodiments, X¹ is —(*C═O)—O-optionally substituted (C₅-C₂₀)alkyl, wherein the atom marked with a * is connected to W¹ or —CH(Y¹)—when W¹ is a covalent bond. In embodiments, X¹ is —(*C═O)—O-optionally substituted (C₅-C₁₃)alkyl, wherein the atom marked with a * is connected to W¹ or —CH(Y¹)—when W¹ is a covalent bond. In embodiments, X¹ is —(*C═O)—O-optionally substituted (C₃-C₂₅)alkenyl, wherein the atom marked with a * is connected to W¹ or —CH(Y¹)—when W¹ is a covalent bond. In embodiments, X¹ is —(*C═O)—O-optionally substituted (C₃-C₂₀)alkenyl, wherein the atom marked with a * is connected to W¹ or —CH(Y¹)—when W¹ is a covalent bond. In embodiments, X¹ is —(*C═O)—O-optionally substituted (C₅-C₂₀)alkenyl, wherein the atom marked with a * is connected to W¹ or —CH(Y¹)—when W¹ is a covalent bond. In embodiments, X¹ is —(*C═O)—O-optionally substituted (C₁₀-C₂₀)alkenyl, wherein the atom marked with a * is connected to W¹ or —CH(Y¹)—when W¹ is a covalent bond. In embodiments, X¹ is —(*C═O)—O-optionally substituted (C₁₀-C₁₃)alkenyl, wherein the atom marked with a * is connected to W¹ or —CH(Y¹)—when W¹ is a covalent bond. In embodiments, X¹ is —*O—(C=O)-optionally substituted (C₃-C₂₅)alkyl, wherein the atom marked with a * is connected to W¹ or —CH(Y¹)—when W¹ is a covalent bond. In embodiments, X¹ is —*O—(C=O)-optionally substituted (C₃-C₂₀)alkyl, wherein the atom marked with a * is connected to W¹ or —CH(Y¹)—when W¹ is a covalent bond. In embodiments, X¹ is —*O—(C=O)-optionally substituted (C₅-C₂₀)alkyl, wherein the atom marked with a * is connected to W¹ or —CH(Y′)— when W¹ is a covalent bond. In embodiments, X¹ is —*O—(C=O)-optionally substituted (C₅-C₁₃)alkyl, wherein the atom marked with a * is connected to W¹ or —CH(Y¹)—when W¹ is a covalent bond. In embodiments, X¹ is —*O—(C=O)-optionally substituted (C₃-C₂₅)alkenyl, wherein the atom marked with a * is connected to W¹ or —CH(Y¹)—when W¹ is a covalent bond. In embodiments, X¹ is —*O—(C=O)-optionally substituted (C₃-C₂₀)alkenyl, wherein the atom marked with a * is connected to W¹ or —CH(Y¹)—when W¹ is a covalent bond. In embodiments, X¹ is —*O—(C=O)-optionally substituted (C₅-C₂₀)alkenyl, wherein the atom marked with a * is connected to W¹ or —CH(Y¹)—when W¹ is a covalent bond. In embodiments, X¹ is —*O—(C═O)-optionally substituted (C₁₀-C₂₀)alkenyl, wherein the atom marked with a * is connected to W¹ or —CH(Y¹)—when W¹ is a covalent bond. In embodiments, X¹ is —*O—(C═O)-optionally substituted (C₁₀-C₁₃)alkenyl, wherein the atom marked with a * is connected to W¹ or —CH(Y¹)—when W¹ is a covalent bond.

In embodiments, R^{2B} is optionally substituted (C₅-C₂₅)alkyl. In embodiments, R^{2B} is optionally substituted (C₅-C₂₀)alkyl. In embodiments, R^{2B} is optionally substituted (C₅-C₁₅)alkyl. In embodiments, R^{2B} is optionally substituted (C₆-C₁₂)alkyl.

In embodiments, R^{2B} is optionally substituted (C₅-C₂₅)alkenyl. In embodiments, R^{2B} is optionally substituted (C₅-C₂₀)alkenyl. In embodiments, R^{2B} is optionally substituted (C₁₀-C₂₀)alkenyl. In embodiments, R^{2B} is optionally substituted (C₁₅-C₂₀)alkenyl.

In embodiments, R^{2B} is —W¹—X¹. In embodiments, W¹ is a covalent bond. In embodiments, W¹ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W⁷ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y¹)—when W¹ is a covalent bond. In embodiments, X¹ is —(*C═O)—O-optionally substituted (C₃-C₂₀)alkyl, wherein the atom marked with a * is connected to W¹ or —CH(Y′)— when W¹ is a covalent bond. In embodiments, X¹ is —(*C═O)—O-optionally substituted (C₅-C₂₀)alkyl, wherein the atom marked with a * is connected to W¹ or —CH(Y¹)—when W¹ is a covalent bond. In embodiments, X¹ is —(*C═O)—O-optionally substituted (C₅-C₁₃)alkyl, wherein the atom marked with a * is connected to W¹ or —CH(Y¹)—when W¹ is a covalent bond. In embodiments, X¹ is —(*C═O)—O-optionally substituted (C₃-C₂₅)alkenyl, wherein the atom marked with a * is connected to W¹ or —CH(Y¹)—when W¹ is a covalent bond. In embodiments, X¹ is —(*C═O)—O-optionally substituted (C₃-C₂₀)alkenyl, wherein the atom marked with a * is connected to W¹ or —CH(Y¹)—when W¹ is a covalent bond. In embodiments, X¹ is —(*C═O)—O-optionally substituted (C₅-C₂₀)alkenyl, wherein the atom marked with a * is connected to W¹ or —CH(Y¹)—when W¹ is a covalent bond. In embodiments, X¹ is —(*C═O)—O-optionally substituted (C₁₀-C₂₀)alkenyl, wherein the atom marked with a * is connected to W¹ or —CH(Y¹)—when W¹ is a covalent bond. In embodiments, X¹ is —(*C═O)—O-optionally substituted (C₁₀-C₁₃)alkenyl, wherein the atom marked with a * is connected to W¹ or —CH(Y¹)—when W¹ is a covalent bond. In embodiments, X¹ is —*O—(C═O)-optionally substituted (C₃-C₂₅)alkyl, wherein the atom marked with a * is connected to W¹ or —CH(Y¹)—when W¹ is a covalent bond. In embodiments, X¹ is —*O—(C═O)-optionally substituted (C₃-C₂₀)alkyl, wherein the atom marked with a * is connected to W¹ or —CH(Y¹)—when W¹ is a covalent bond. In embodiments, X¹ is —*O—(C═O)-optionally substituted (C₅-C₂₀)alkyl, wherein the atom marked with a * is connected to W¹ or —CH(Y¹)— when W¹ is a covalent bond. In embodiments, X¹ is —*O—(C═O)-optionally substituted (C₅-C₁₃)alkyl, wherein the atom marked with a * is connected to W¹ or —CH(Y¹)—when W¹ is a covalent bond. In embodiments, X¹ is —*O—(C═O)-optionally substituted (C₃-C₂₅)alkenyl, wherein the atom marked with a * is connected to W¹ or —CH(Y¹)—when W¹ is a covalent bond. In embodiments, X¹ is —*O—(C═O)-optionally substituted (C₃-C₂₀)alkenyl, wherein the atom marked with a * is connected to W¹ or —CH(Y¹)—when W¹ is a covalent bond. In embodiments, X¹ is —*O—(C═O)-optionally substituted (C₅-C₂₀)alkenyl, wherein the atom marked with a * is connected to W¹ or —CH(Y¹)—when W¹ is a covalent bond. In embodiments, X¹ is —*O—(C═O)-optionally substituted (C₁₀-C₂₀)alkenyl, wherein the atom marked with a * is connected to W¹ or —CH(Y¹)—when W¹ is a covalent bond. In embodiments, X¹ is —*O—(C═O)-optionally substituted (C₁₀-C₁₃)alkenyl, wherein the atom marked with a * is connected to W¹ or —CH(Y¹)—when W¹ is a covalent bond.

In embodiments, R^{2C} is optionally substituted (C₅-C₂₅)alkyl. In embodiments, R^{2C} is optionally substituted (C₅-C₂₀)alkyl. In embodiments, R^{2C} is optionally substituted (C₅-C₁₅)alkyl. In embodiments, R^{2C} is optionally substituted (C₆-C₁₂)alkyl.

In embodiments, R^{2C} is optionally substituted (C₅-C₂₅)alkenyl. In embodiments, R^{2C} is optionally substituted (C₅-C₂₀)alkenyl. In embodiments, R^{2C} is optionally substituted (C₁₀-C₂₀)alkenyl. In embodiments, R^{2C} is optionally substituted (C₁₅-C₂₀)alkenyl.

In embodiments, R^{2C} is —W¹—X¹. In embodiments, W¹ is a covalent bond. In embodiments, W¹ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W⁷ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y¹)—when W¹ is a covalent bond. In embodiments, X¹ is —(*C═O)—O-optionally substituted (C₃-C₂₀)alkyl, wherein the atom marked with a * is connected to W¹ or —CH(Y′)— when W¹ is a covalent bond. In embodiments, X¹ is —(*C═O)—O-optionally substituted (C₅-C₂₀)alkyl, wherein the atom marked with a * is connected to W¹ or —CH(Y¹)—when W¹ is a covalent bond. In embodiments, X¹ is —(*C═O)—O-optionally substituted (C₅-C₁₃)alkyl, wherein the atom marked with a * is connected to W¹ or —CH(Y¹)—when W¹ is a covalent bond. In embodiments, X¹ is —(*C═O)—O-optionally substituted (C₃-C₂₅)alkenyl, wherein the atom marked with a * is connected to W¹ or —CH(Y¹)—when W¹ is a covalent bond. In embodiments, X¹ is —(*C═O)—O-optionally substituted (C₃-C₂₀)alkenyl, wherein the atom marked with a * is connected to W¹ or —CH(Y¹)—when W¹ is a covalent bond. In embodiments, X¹ is —(*C═O)—O-optionally substituted (C₅-C₂₀)alkenyl, wherein the atom marked with a * is connected to W¹ or —CH(Y¹)—when W¹ is a covalent bond. In embodiments, X¹ is —(*C═O)—O-optionally substituted (C₁₀-C₂₀)alkenyl, wherein the atom marked with a * is connected to W¹ or —CH(Y¹)—when W¹ is a covalent bond. In embodiments, X¹ is —(*C═O)—O-optionally substituted (C₁₀-C₁₃)alkenyl, wherein the atom marked with a * is connected to W¹ or —CH(Y¹)—when W¹ is a covalent bond. In embodiments, X¹ is —*O—(C═O)-optionally substituted (C₃-C₂₅)alkyl, wherein the atom marked with a * is connected to W¹ or —CH(Y¹)—when W¹ is a covalent bond. In embodiments, X¹ is —*O—(C═O)-optionally substituted (C₃-C₂₀)alkyl, wherein the atom marked with a * is connected to W¹ or —CH(Y¹)—when W¹ is a covalent bond. In embodiments, X¹ is —*O—(C═O)-optionally substituted (C₅-C₂₀)alkyl, wherein the atom marked with a * is connected to W¹ or —CH(Y′)— when W¹ is a covalent bond. In embodiments, X¹ is —*O—(C═O)-optionally substituted (C₅-C₁₃)alkyl, wherein the atom marked with a * is connected to W¹ or —CH(Y¹)—when W¹ is a covalent bond. In embodiments, X¹ is —*O—(C═O)-optionally substituted (C₃-C₂₅)alkenyl, wherein the atom marked with a * is connected to W¹ or —CH(Y¹)—when W¹ is a covalent bond. In embodiments, X¹ is —*O—(C═O)-optionally substituted (C₃-C₂₀)alkenyl, wherein the atom marked with a * is connected to W¹ or —CH(Y¹)—when W¹ is a covalent bond. In embodiments, X¹ is —*O—(C═O)-optionally substituted (C₅-C₂₀)alkenyl, wherein the atom marked with a * is connected to W¹ or —CH(Y¹)—when W¹ is a covalent bond. In embodiments, X¹ is —*O—(C═O)-optionally substituted (C₁₀-C₂₀)alkenyl, wherein the atom marked with a * is connected to W¹ or —CH(Y¹)—when W¹ is a covalent bond. In embodiments, X¹ is —*O—(C═O)-optionally substituted (C₁₀-C₁₃)alkenyl, wherein the atom marked with a * is connected to W¹ or —CH(Y¹)—when W¹ is a covalent bond.

In embodiments, R^{2D} is optionally substituted (C₅-C₂₅)alkyl. In embodiments, R^{2D} is optionally substituted (C₅-C₂₀)alkyl. In embodiments, R^{2D} is optionally substituted (C₅-C₁₅)alkyl. In embodiments, R^{2D} is optionally substituted (C₆-C₁₂)alkyl.

In embodiments, R^{2D} is optionally substituted (C₅-C₂₅)alkenyl. In embodiments, R^{2D} is optionally substituted (C₅-C₂₀)alkenyl. In embodiments, R^{2D} is optionally substituted (C₁₀-C₂₀)alkenyl. In embodiments, R^{2D} is optionally substituted (C₁₅-C₂₀)alkenyl.

In embodiments, R^{2D} is —W¹—X¹. In embodiments, W¹ is a covalent bond. In embodiments, W¹ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W⁷ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y¹)—when W¹ is a covalent bond. In embodiments, X¹ is —(*C═O)—O-optionally substituted (C₃-C₂₀)alkyl, wherein the atom marked with a * is connected to W¹ or —CH(Y′)— when W¹ is a covalent bond. In embodiments, X¹ is —(*C═O)—O-optionally substituted (C₅-C₂₀)alkyl, wherein the atom marked with a * is connected to W¹ or —CH(Y¹)—when W¹ is a covalent bond. In embodiments, X¹ is —(*C═O)—O-optionally substituted (C₅-C₁₃)alkyl, wherein the atom marked with a * is connected to W¹ or —CH(Y¹)—when W¹ is a covalent bond. In embodiments, X¹ is —(*C═O)—O-optionally substituted (C₃-C₂₅)alkenyl, wherein the atom marked with a * is connected to W¹ or —CH(Y¹)—when W¹ is a covalent bond. In embodiments, X¹ is —(*C═O)—O-optionally substituted (C₃-C₂₀)alkenyl, wherein the atom marked with a * is connected to W¹ or —CH(Y¹)—when W¹ is a covalent bond. In embodiments, X¹ is —(*C═O)—O-optionally substituted (C₅-C₂₀)alkenyl, wherein the atom marked with a * is connected to W¹ or —CH(Y¹)—when W¹ is a covalent bond. In embodiments, X¹ is —(*C═O)—O-optionally substituted (C₁₀-C₂₀)alkenyl, wherein the atom marked with a * is connected to W¹ or —CH(Y¹)—when W¹ is a covalent bond. In embodiments, X¹ is —(*C═O)—O-optionally substituted (C₁₀-C₁₃)alkenyl, wherein the atom marked with a * is connected to W¹ or —CH(Y¹)—when W¹ is a covalent bond. In embodiments, X¹ is —*O—(C═O)-optionally substituted (C₃-C₂₅)alkyl, wherein the atom marked with a * is connected to W¹ or —CH(Y¹)—when W¹ is a covalent bond. In embodiments, X¹ is —*O—(C═O)-optionally substituted (C₃-C₂₀)alkyl, wherein the atom marked with a * is connected to W¹ or —CH(Y¹)—when W¹ is a covalent bond. In embodiments, X¹ is —*O—(C═O)-optionally substituted (C₅-C₂₀)alkyl, wherein the atom marked with a * is connected to W¹ or —CH(Y¹)—when W¹ is a covalent bond. In embodiments, X¹ is —*O—(C═O)-optionally substituted (C₅-C₁₃)alkyl, wherein the atom marked with a * is connected to W¹ or —CH(Y¹)—when W¹ is a covalent bond. In embodiments, X¹ is —*O—(C═O)-optionally substituted (C₃-C₂₅)alkenyl, wherein the atom marked with a * is connected to W¹ or —CH(Y¹)—when W¹ is a covalent bond. In embodiments, X¹ is —*O—(C═O)-optionally substituted (C₃-C₂₀)alkenyl, wherein the atom marked with a * is connected to W¹ or —CH(Y¹)—when W¹ is a covalent bond. In embodiments, X¹ is —*O—(C═O)-optionally substituted (C₅-C₂₀)alkenyl, wherein the atom marked with a * is connected to W¹ or —CH(Y¹)—when W¹ is a covalent bond. In embodiments, X¹ is —*O—(C═O)-optionally substituted (C₁₀-C₂₀)alkenyl, wherein the atom marked with a * is connected to W¹ or —CH(Y¹)—when W¹ is a covalent bond. In embodiments, X¹ is —*O—(C═O)-optionally substituted (C₁₀-C₁₃)alkenyl, wherein the atom marked with a * is connected to W¹ or —CH(Y¹)—when W¹ is a covalent bond.

In embodiments, each R^{2A}, R^{2B}, R^{2C} and R^{2D} is independently selected from:

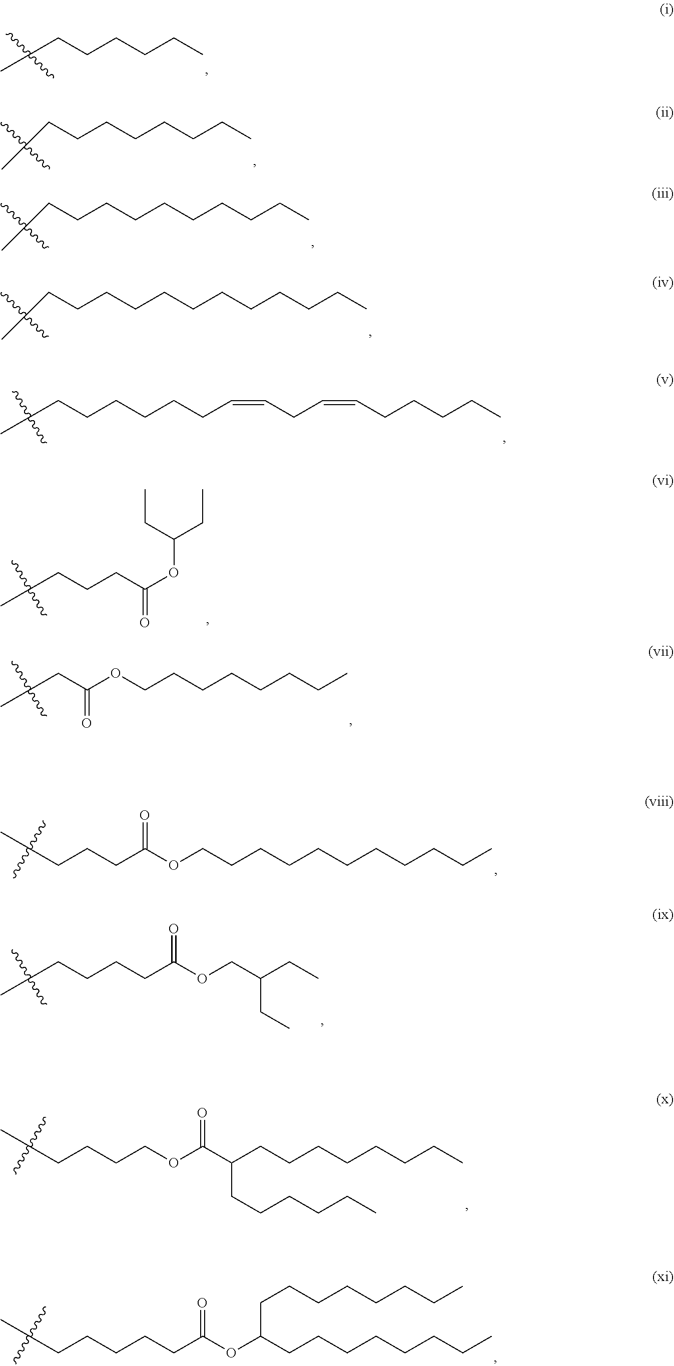

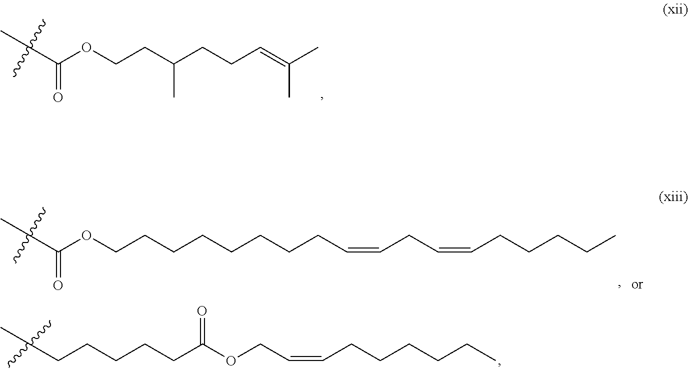 [SMILES: *C(=O)OCCC(C)CCC=C(C)C ; *C(=O)OCCCCCCCC/C=C\C/C=C\CCCCC ; *CCCCCC(=O)OC/C=C\CCCCCC]

preferably wherein each R^{2A}, R^{2B}, R^{2C} and R^{2D} is independently selected from options (ii), (iii), (iv), (v), (vi), (viii), (ix), (x), (xi), (xii), (xiii) or (xiv).

In embodiments, each R^{2A}, R^{2B}, R^{2C} and R^{2D} is independently selected from:

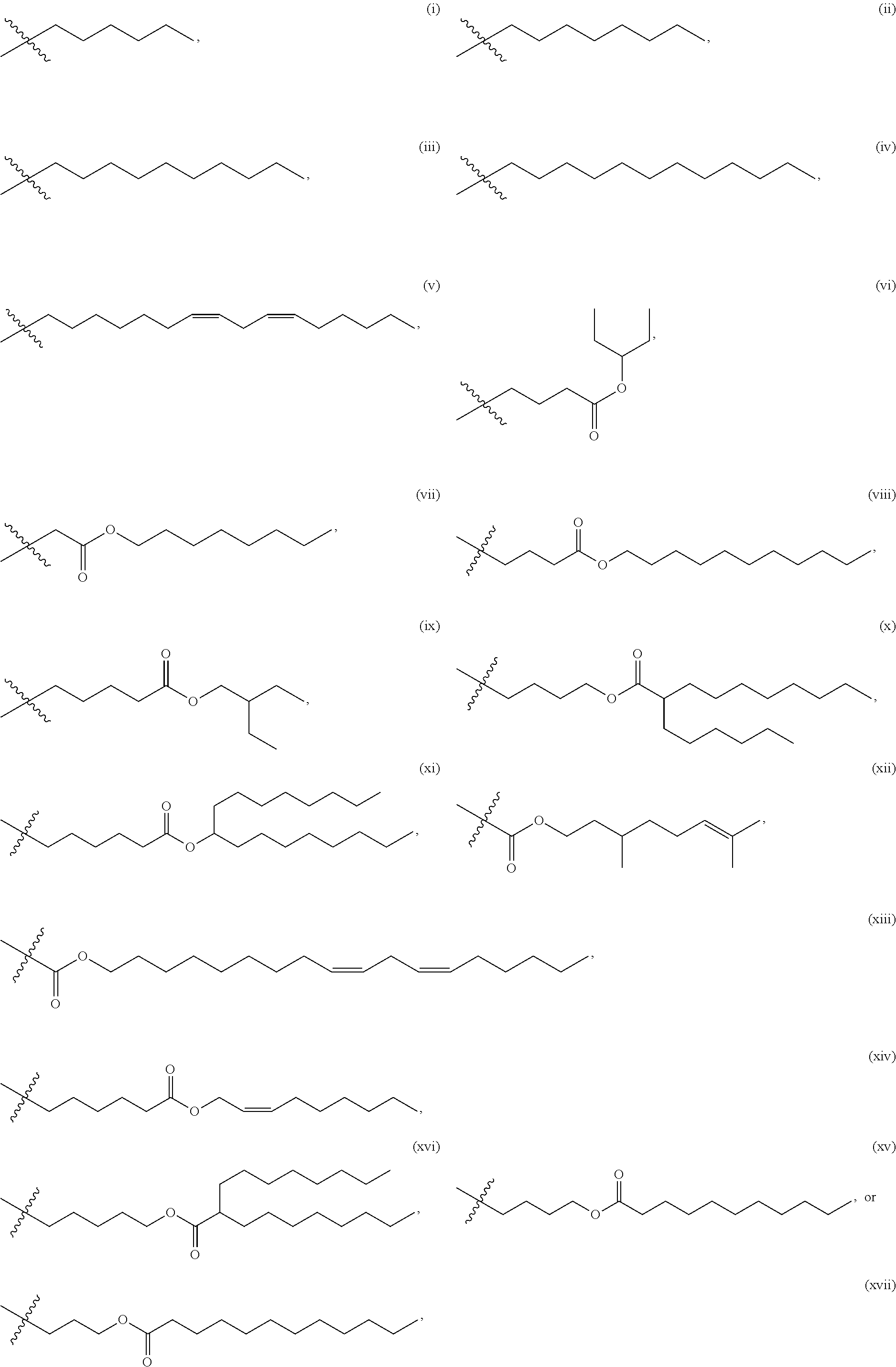

preferably wherein each R^{2A}, R^{2B}, R^{2C} and R^{2D} is independently selected from options (ii), (iii), (iv), (v), (vi), (viii), (ix), (x), (xi), (xii), (xiii), (xiv), (xv), (xvi) or (xvii).

In embodiments, R^{2A}, R^{2B}, R^{2C} and R^{2D} are the same. In embodiments, R^{2A} and R^{2B} are the same and R^{2C} and R^{2D} are the same. In embodiments, R^{2A} and R^{2C} are the same and R^{2B} and R^{2D} are the same. In embodiments, R^{2A} and R^{2C} are the same and R^{2B} and R^{2D} are different.

The cationic lipids of the present invention also include compounds having a structure according to Formula (II):

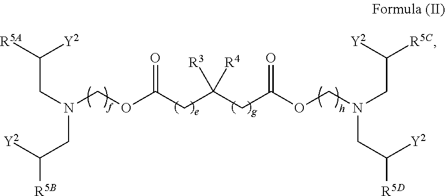

or a pharmaceutically acceptable salt thereof,
wherein R³ is selected from hydrogen, or optionally substituted (C₁-C₆)alkyl;
R⁴ is selected from hydrogen, —OH, —NH₂, optionally substituted (C₁-C₆)alkyl, optionally substituted aryl, optionally substituted heteroaryl, optionally substituted (C₁-C₃)alkylene-optionally substituted aryl, or optionally substituted (C₁-C₃)alkylene-optionally substituted heteroaryl;
e and g are integers that are each independently selected from 0, 1, 2, 3, or 4;
f and h are integers that are each independently selected from 1, 2, 3, 4, 5 or 6;
each Y² is independently selected from hydrogen or —OH;
R^{5A}, R^{5B}, R^{5C} and R^{5D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y²)—when W¹ is a covalent bond.

In embodiments, the cationic lipids of the present invention include compounds having a structure according to Formula (IIA):

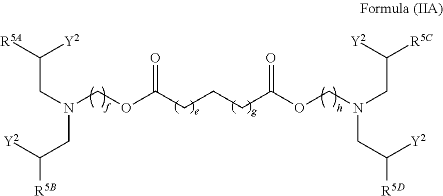

or a pharmaceutically acceptable salt thereof.

In embodiments, the cationic lipids of the present invention include compounds having a structure according to Formula (II) wherein Y²=OH. In embodiments, the cationic lipids of the present invention include compounds having a structure according to Formula (IIA1):

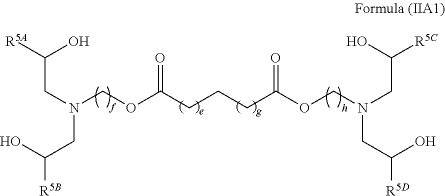

or a pharmaceutically acceptable salt thereof.

In embodiments, the cationic lipids of the present invention include compounds having a structure according to Formula (II) wherein Y²=OH, R³=hydrogen, and e and g=0. In embodiments, the cationic lipids of the present invention include compounds having a structure according to Formula (II) wherein Y²=OH, R⁴=hydrogen, and e and g=0. In embodiments, the cationic lipids of the present invention include compounds having a structure according to Formula (II) wherein Y²=OH, R³ and R⁴=hydrogen, and e and g=0. In embodiments, the cationic lipids of the present invention include compounds having a structure according to Formula (IIA1i):

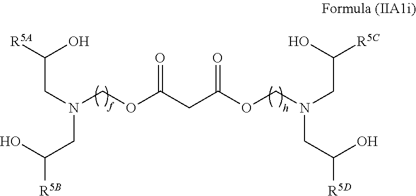

or a pharmaceutically acceptable salt thereof.

In embodiments, the cationic lipids of the present invention include compounds having a structure according to Formula (II) wherein Y²=hydrogen. In embodiments, the cationic lipids of the present invention include compounds having a structure according to Formula (IIA2):

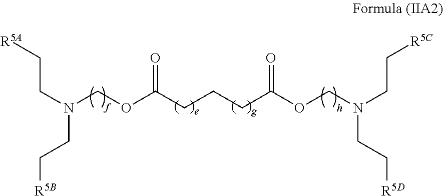

or a pharmaceutically acceptable salt thereof.

In embodiments, the cationic lipids of the present invention include compounds having a structure according to Formula (II) wherein R³═H. In embodiments, the cationic lipids of the present invention include compounds having a structure according to Formula (IIB):

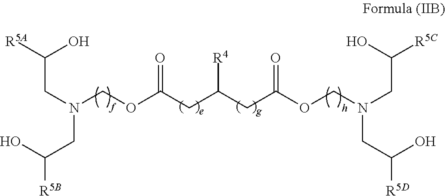

or a pharmaceutically acceptable salt thereof, wherein R⁴ is selected from (C₁-C₆)alkyl, phenyl or benzyl. In embodiments, R⁴ is selected from methyl, isopropyl, phenyl or benzyl. In embodiments, R⁴ is methyl. In embodiments, R⁴ is isopropyl. In embodiments, R⁴ is phenyl. In embodiments, R⁴ is benzyl.

In embodiments, the cationic lipids of the present invention include compounds having a structure according to Formula (II) wherein R³=methyl. In embodiments, the cationic lipids of the present invention include compounds having a structure according to Formula (IIC):

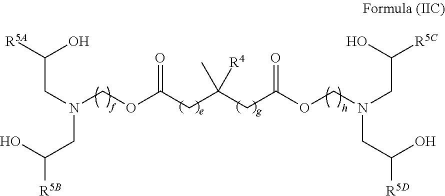

or a pharmaceutically acceptable salt thereof, wherein R⁴ is selected from (C₁-C₆)alkyl. In embodiments, R⁴ is selected from methyl or ethyl. In embodiments, R⁴ is methyl. In embodiments, R⁴ is ethyl.

In embodiments, the cationic lipids of the present invention include compounds having a structure according to Formula (II) wherein R⁴=OH. In embodiments, the cationic lipids of the present invention include compounds having a structure according to Formula (II) wherein R³=methyl and R⁴=OH. In embodiments, the cationic lipids of the present invention include compounds having a structure according to Formula (IIC1):

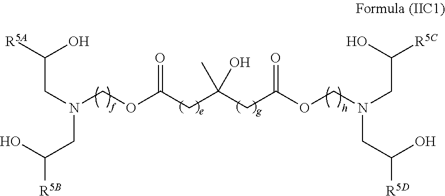

or a pharmaceutically acceptable salt thereof.

In embodiments, the cationic lipids of the present invention include compounds having a structure according to Formula (II) wherein e=1 and g=1. In embodiments, the cationic lipids of the present invention include compounds having a structure according to Formula (II) wherein R³=methyl, e=1 and g=1. In embodiments, the cationic lipids of the present invention include compounds having a structure according to Formula (II) wherein R⁴=OH, e=1 and g=1. In embodiments, the cationic lipids of the present invention include compounds having a structure according to Formula (II) wherein R³=methyl, R⁴=OH, e=1 and g=1. In embodiments, the cationic lipids of the present invention include compounds having a structure according to Formula (IIC1i):

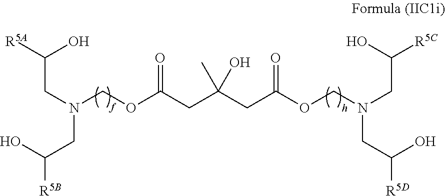

or a pharmaceutically acceptable salt thereof.

In embodiments, the cationic lipids of the present invention include compounds having a structure according to Formula (II) wherein Y²=OH and R³=hydrogen. In embodiments, the cationic lipids of the present invention include compounds having a structure according to Formula (II) wherein Y²=OH and R⁴=OH. In embodiments, the cationic lipids of the present invention include compounds having a structure according to Formula (II) wherein Y²=OH, R³=hydrogen and R⁴=OH. In embodiments, the cationic lipids of the present invention include compounds having a structure according to Formula (IID):

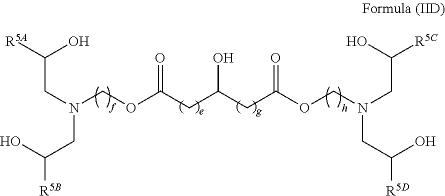

or a pharmaceutically acceptable salt thereof.

In embodiments, the cationic lipids of the present invention include compounds having a structure according to Formula (II) wherein Y²=OH, R³=hydrogen, and e and g=0. In embodiments, the cationic lipids of the present invention include compounds having a structure according to Formula (II) wherein Y²=OH, R⁴=OH, and e and g=0. In embodiments, the cationic lipids of the present invention include compounds having a structure according to Formula (II) wherein Y²=OH, R³=hydrogen, R⁴=OH, and e and g=0. In embodiments, the cationic lipids of the present invention include compounds having a structure according to Formula (IID1):

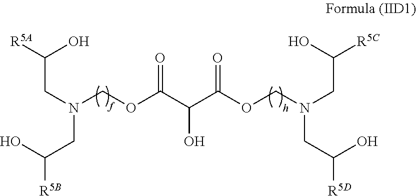

or a pharmaceutically acceptable salt thereof.

In embodiments, the cationic lipids of the present invention include compounds having a structure according to Formula (II) wherein Y²=OH and R³=hydrogen. In embodiments, the cationic lipids of the present invention include compounds having a structure according to Formula (II) wherein Y²=OH and R⁴=NH₂. In embodiments, the cationic lipids of the present invention include compounds having a structure according to Formula (II) wherein Y²=OH, R³=hydrogen and R⁴=NH₂. In embodiments, the cationic lipids of the present invention include compounds having a structure according to Formula (IIE):

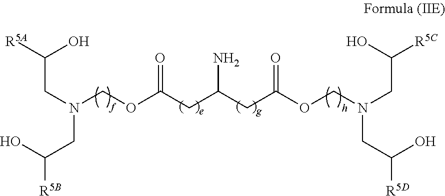

or a pharmaceutically acceptable salt thereof.

In embodiments, the cationic lipids of the present invention include compounds having a structure according to Formula (II) wherein Y²=OH, R³=hydrogen, and e and g=0. In embodiments, the cationic lipids of the present invention include compounds having a structure according to Formula (II) wherein Y²=OH, R⁴=NH₂, and e and g=0. In embodiments, the cationic lipids of the present invention include compounds having a structure according to Formula (II) wherein Y²=OH, R³=hydrogen, R⁴=NH₂, and e and g=0. In embodiments, the cationic lipids of the present invention include compounds having a structure according to Formula (IIE1):

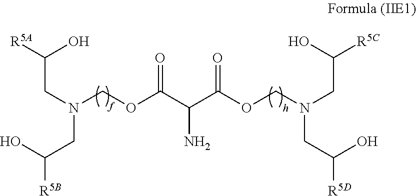

or a pharmaceutically acceptable salt thereof.

In embodiments, the cationic lipids of the present invention include compounds having a structure according to Formula (IIC1ii):

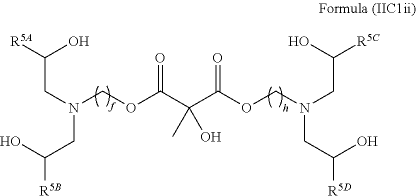

or a pharmaceutically acceptable salt thereof.

In embodiments, the cationic lipids of the present invention include compounds having a structure according to Formula (IIC2):

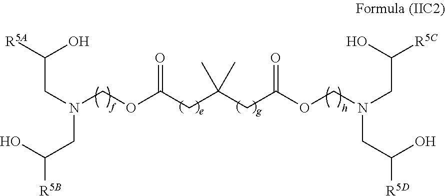

or a pharmaceutically acceptable salt thereof.

In embodiments, the cationic lipids of the present invention include compounds having a structure according to Formula (IIC2i):

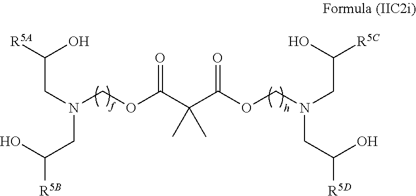

or a pharmaceutically acceptable salt thereof.

In embodiments, R³ is hydrogen. In embodiments, R³ is optionally substituted (C₁-C₆)alkyl. In embodiments, R³ is methyl.

In embodiments, R⁴ is selected from hydrogen, —OH, —NH₂, optionally substituted (C₁-C₆)alkyl, optionally substituted phenyl, or optionally substituted (C₁-C₃)alkylene-optionally substituted phenyl. In embodiments, R⁴ is hydrogen. In embodiments, R⁴ is —OH. In embodiments, R⁴ is —NH₂. In embodiments, R⁴ is optionally substituted (C₁-C₆)alkyl. In embodiments, R⁴ is methyl. In embodiments, R⁴ is ethyl. In embodiments, R⁴ is isopropyl. In embodiments, R⁴ is optionally substituted aryl. In embodiments, R⁴ is optionally substituted phenyl. In embodiments, R⁴ is phenyl. In embodiments, R⁴ is optionally substituted (C₁-C₃)alkylene-optionally substituted aryl. In embodiments, R⁴ is optionally substituted (C₁-C₃)alkylene-optionally substituted phenyl. In embodiments, R⁴ is optionally substituted benzyl. In embodiments, R⁴ is benzyl. In embodiments, R⁴ is optionally substituted heteroaryl. In embodiments, R⁴ is optionally substituted (C₁-C₃)alkylene-optionally substituted heteroaryl.

In embodiments, e is 0, 1 or 2, preferably wherein the cationic lipid has a structure according to any one of Formula (IIA), Formula (IIA1) or Formula (IIB). In embodiments, e is 1, preferably wherein the cationic lipid has a structure according to any one of Formula (IIA2) or Formula (IIC). In embodiments, e is 0, preferably wherein the cationic lipid has a structure according to any one of (i) Formula (IIA1i), Formula (IID), or Formula (IIE), or (ii) Formula (IIC1), Formula (IIC2) or Formula (IIE). In embodiments, e=2. In embodiments, e=3. In embodiments, e=4.

In embodiments, f is 3, 4, 5 or 6, preferably wherein the cationic lipid has a structure according to Formula (IIC1i). In embodiments, f is 3, 4 or 5, preferably wherein the cationic lipid has a structure according to Formula (IIA1i). In embodiments, f=3 or 4, preferably wherein the cationic lipid has a structure according to Formula (IID1). In embodiments, f is 3, preferably wherein the cationic lipid has a structure according to any one of (i) Formula (IIA), Formula (IIA1), Formula (IIA2), Formula (IIB), Formula (IIC), or Formula (IIE1), or (ii) Formula (IIC1), Formula (IIC1ii), Formula (IIE) or Formula (IIE1). In embodiments, f is 4, preferably wherein the cationic lipid has a structure according to Formula (IIC2) or Formula (IIC2i). In embodiments, f=1. In embodiments, f=2. In embodiments, f=4. In embodiments, f=5. In embodiments, f=6.

In embodiments, g is 0 or 1 preferably wherein the cationic lipid has a structure according to any one of Formula (IIA), Formula (IIA1) or Formula (IIB). In embodiments, g is 0, preferably wherein the cationic lipid has a structure according to any one of (i) Formula (IIA1i), Formula (IIA2), Formula (IID), or Formula (IIE), or (ii) Formula (IIC1), Formula (IIC2) or Formula (IIE). In embodiments, g is 1, preferably wherein the cationic lipid has a structure according to Formula (IIC). In embodiments, g is 2. In embodiments, g is 3. In embodiments, g is 4.

In embodiments, h is 3, 4, 5 or 6, preferably wherein the cationic lipid has a structure according to Formula (IIC1i). In embodiments, h is 3, 4 or 5, preferably wherein the cationic lipid has a structure according to Formula (IIA1i). In embodiments, h is 3 or 4, preferably wherein the cationic lipid has a structure according to Formula (IID1). In embodiments, h is 3, preferably wherein the cationic lipid has a structure according to any one of (i) Formula (IIA), Formula (IIA1), Formula (IIA2), Formula (IIB), Formula (IIC), or Formula (IIE1), or (ii) Formula (IIC1), Formula (IIC1ii), Formula (IIE) or Formula (IIE1). In embodiments, wherein h is 4, preferably wherein the cationic lipid has a structure according to Formula (IIC2) or Formula (IIC2i). In embodiments, h is 1. In embodiments, h is 2. In embodiments, h is 4. In embodiments, h is 5. In embodiments, h is 6.

In embodiments, at least one Y² is —OH. In embodiments, at least one Y² is hydrogen. In embodiments, Y² is —OH. In embodiments, Y² is hydrogen.

In embodiments, R^{5A} is optionally substituted (C₅-C₂₅)alkyl. In embodiments, R^{5A} is optionally substituted (C₅-C₂₀)alkyl. In embodiments, R^{5A} is optionally substituted (C₅-C₁₅)alkyl. In embodiments, R^{5A} is optionally substituted (C₆-C₁₂)alkyl.

In embodiments, R^{5A} is optionally substituted (C₅-C₂₅)alkenyl. In embodiments, R^{5A} is optionally substituted (C₅-C₂₀)alkenyl. In embodiments, R^{5A} is optionally substituted (C₁₀-C₂₀)alkenyl. In embodiments, R^{5A} is optionally substituted (C₁₅-C₂₀)alkenyl.

In embodiments, R^{5A} is —W¹—X¹. In embodiments, W¹ is a covalent bond. In embodiments, W¹ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W⁷ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y²)—when W¹ is a covalent bond. In embodiments, X¹ is —(*C═O)—O-optionally substituted (C₃-C₂₀)alkyl, wherein the atom marked with a * is connected to W¹ or —CH(Y²)— when W¹ is a covalent bond. In embodiments, X¹ is —(*C═O)—O-optionally substituted (C₅-C₂₀)alkyl, wherein the atom marked with a * is connected to W¹ or —CH(Y²)—when W¹ is a covalent bond. In embodiments, X¹ is —(*C═O)—O-optionally substituted (C₅-C₁₃)alkyl, wherein the atom marked with a * is connected to W¹ or —CH(Y²)—when W¹ is a covalent bond. In embodiments, X¹ is —(*C═O)—O-optionally substituted (C₃-C₂₅)alkenyl, wherein the atom marked with a * is connected to W¹ or —CH(Y²)—when W¹ is a covalent bond. In embodiments, X¹ is —(*C═O)—O-optionally substituted (C₃-C₂₀)alkenyl, wherein the atom marked with a * is connected to W¹ or —CH(Y²)—when W¹ is a covalent bond. In embodiments, X¹ is —(*C═O)—O-optionally substituted (C₅-C₂₀)alkenyl, wherein the atom marked with a * is connected to W¹ or —CH(Y²)—when W¹ is a covalent bond. In embodiments, X¹ is —(*C═O)—O-optionally substituted (C₁₀-C₂₀)alkenyl, wherein the atom marked with a * is connected to W¹ or —CH(Y²)—when W¹ is a covalent bond. In embodiments, X¹ is —(*C═O)—O-optionally substituted (C₁₀-C₁₃)alkenyl, wherein the atom marked with a * is connected to W¹ or —CH(Y²)—when W¹ is a covalent bond. In embodiments, X¹ is —*O—(C═O)-optionally substituted (C₃-C₂₅)alkyl, wherein the atom marked with a * is connected to W¹ or —CH(Y²)—when W¹ is a covalent bond. In embodiments, X¹ is —*O—(C═O)-optionally substituted (C₃-C₂₀)alkyl, wherein the atom marked with a * is connected to W¹ or —CH(Y²)—when W¹ is a covalent bond. In embodiments, X¹ is —*O—(C═O)-optionally substituted (C₅-C₂₀)alkyl, wherein the atom marked with a * is connected to W¹ or —CH(Y²)— when W¹ is a covalent bond. In embodiments, X¹ is —*O—(C═O)-optionally substituted (C₅-C₁₃)alkyl, wherein the atom marked with a * is connected to W¹ or —CH(Y²)—when W¹ is a covalent bond. In embodiments, X¹ is —*O—(C═O)-optionally substituted (C₃-C₂₅)alkenyl, wherein the atom marked with a * is connected to W¹ or —CH(Y²)—when W¹ is a covalent bond. In embodiments, X¹ is —*O—(C═O)-optionally substituted (C₃-C₂₀)alkenyl, wherein the atom marked with a * is connected to W¹ or —CH(Y²)—when W¹ is a covalent bond. In embodiments, X¹ is —*O—(C═O)-optionally substituted (C₅-C₂₀)alkenyl, wherein the atom marked with a * is connected to W¹ or —CH(Y²)—when W¹ is a covalent bond. In embodiments, X¹ is —*O—(C═O)-optionally substituted (C₁₀-C₂₀)alkenyl, wherein the atom marked with a * is connected to W¹ or —CH(Y²)—when W¹ is a covalent bond. In embodiments, X¹ is —*O—(C═O)-optionally substituted (C₁₀-C₁₃)alkenyl, wherein the atom marked with a * is connected to W¹ or —CH(Y²)—when W¹ is a covalent bond.

In embodiments, R^{5B} is optionally substituted (C₅-C₂₅)alkyl. In embodiments, R^{5B} is optionally substituted (C₅-C₂₀)alkyl. In embodiments, R^{5B} is optionally substituted (C₅-C₁₅)alkyl. In embodiments, R^{5B} is optionally substituted (C₆-C₁₂)alkyl.

In embodiments, R^{5B} is optionally substituted (C₅-C₂₅)alkenyl. In embodiments, R^{5B} is optionally substituted (C₅-C₂₀)alkenyl. In embodiments, R^{5B} is optionally substituted (C₁₀-C₂₀)alkenyl. In embodiments, R^{5B} is optionally substituted (C₁₅-C₂₀)alkenyl.

In embodiments, R^{5B} is —W¹—X¹. In embodiments, W¹ is a covalent bond. In embodiments, W⁷ is optionally substituted (C₁-C₁₀)alkylene. In embodiments, W⁷ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W¹ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y²)—when W¹ is a covalent bond. In embodiments, X¹ is —(*C═O)—O-optionally substituted (C₃-C₂₀)alkyl, wherein the atom marked with a * is connected to W¹ or —CH(Y²)— when W¹ is a covalent bond. In embodiments, X¹ is —(*C═O)—O-optionally substituted (C₅-C₂₀)alkyl, wherein the atom marked with a * is connected to W¹ or —CH(Y²)—when W¹ is a covalent bond. In embodiments, X¹ is —(*C═O)—O-optionally substituted (C₅-C₁₃)alkyl, wherein the atom marked with a * is connected to W¹ or —CH(Y²)—when W¹ is a covalent bond. In embodiments, X¹ is —(*C═O)—O-optionally substituted (C₃-C₂₅)alkenyl, wherein the atom marked with a * is connected to W¹ or —CH(Y²)—when W¹ is a covalent bond. In embodiments, X¹ is —(*C═O)—O-optionally substituted (C₃-C₂₀)alkenyl, wherein the atom marked with a * is connected to W¹ or —CH(Y²)—when W¹ is a covalent bond. In embodiments, X¹ is —(*C═O)—O-optionally substituted (C₅-C₂₀)alkenyl, wherein the atom marked with a * is connected to W¹ or —CH(Y²)—when W¹ is a covalent bond. In embodiments, X¹ is —(*C═O)—O-optionally substituted (C₁₀-C₂₀)alkenyl, wherein the atom marked with a * is connected to W¹ or —CH(Y²)—when W¹ is a covalent bond. In embodiments, X¹ is —(*C═O)—O-optionally substituted (C₁₀-C₁₃)alkenyl, wherein the atom marked with a * is connected to W¹ or —CH(Y²)—when W¹ is a covalent bond. In embodiments, X¹ is —*O—(C═O)-optionally substituted (C₃-C₂₅)alkyl, wherein the atom marked with a * is connected to W¹ or —CH(Y²)—when W¹ is a covalent bond. In embodiments, X¹ is —*O—(C═O)-optionally substituted (C₃-C₂₀)alkyl, wherein the atom marked with a * is connected to W¹ or —CH(Y²)—when W¹ is a covalent bond. In embodiments, X¹ is —*O—(C═O)-optionally substituted (C₅-C₂₀)alkyl, wherein the atom marked with a * is connected to W¹ or —CH(Y²)— when W¹ is a covalent bond. In embodiments, X¹ is —*O—(C═O)-optionally substituted (C₅-C₁₃)alkyl, wherein the atom marked with a * is connected to W¹ or —CH(Y²)—when W¹ is a covalent bond. In embodiments, X¹ is —*O—(C═O)-optionally substituted (C₃-C₂₅)alkenyl, wherein the atom marked with a * is connected to W¹ or —CH(Y²)—when W¹ is a covalent bond. In embodiments, X¹ is —*O—(C═O)-optionally substituted (C₃-C₂₀)alkenyl, wherein the atom marked with a * is connected to W¹ or —CH(Y²)—when W¹ is a covalent bond. In embodiments, X¹ is —*O—(C═O)-optionally substituted (C₅-C₂₀)alkenyl, wherein the atom marked with a * is connected to W¹ or —CH(Y²)—when W¹ is a covalent bond. In embodiments, X¹ is —*O—(C═O)-optionally substituted (C₁₀-C₂₀)alkenyl, wherein the atom marked with a * is connected to W¹ or —CH(Y²)—when W¹ is a covalent bond. In embodiments, X¹ is —*O—(C═O)-optionally substituted (C₁₀-C₁₃)alkenyl, wherein the atom marked with a * is connected to W¹ or —CH(Y²)—when W¹ is a covalent bond.

In embodiments, R^{5C} is optionally substituted (C₅-C₂₅)alkyl. In embodiments, R^{5C} is optionally substituted (C₅-C₂₀)alkyl. In embodiments, R^{5C} is optionally substituted (C₅-C₁₅)alkyl. In embodiments, R^{5C} is optionally substituted (C₆-C₁₂)alkyl.

In embodiments, R^{5C} is optionally substituted (C₅-C₂₅)alkenyl. In embodiments, R^{5C} is optionally substituted (C₅-C₂₀)alkenyl. In embodiments, R^{5C} is optionally substituted (C₁₀-C₂₀)alkenyl. In embodiments, R^{5C} is optionally substituted (C₁₅-C₂₀)alkenyl.

In embodiments, R^{5C} is —W¹—X¹. In embodiments, W¹ is a covalent bond. In embodiments, W⁷ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y²)—when W¹ is a covalent bond. In embodiments, X¹ is —(*C═O)—O-optionally substituted (C₃-C₂₀)alkyl, wherein the atom marked with a * is connected to W¹ or —CH(Y²)— when W¹ is a covalent bond. In embodiments, X¹ is —(*C═O)—O-optionally substituted (C₅-C₂₀)alkyl, wherein the atom marked with a * is connected to W¹ or —CH(Y²)—when W¹ is a covalent bond. In embodiments, X¹ is —(*C═O)—O-optionally substituted (C₅-C₁₃)alkyl, wherein the atom marked with a * is connected to W¹ or —CH(Y²)—when W¹ is a covalent bond. In embodiments, X¹ is —(*C═O)—O-optionally substituted (C₃-C₂₅)alkenyl, wherein the atom marked with a * is connected to W¹ or —CH(Y²)—when W¹ is a covalent bond. In embodiments, X¹ is —(*C═O)—O-optionally substituted (C₃-C₂₀)alkenyl, wherein the atom marked with a * is connected to W¹ or —CH(Y²)—when W¹ is a covalent bond. In embodiments, X¹ is —(*C═O)—O-optionally substituted (C₅-C₂₀)alkenyl, wherein the atom marked with a * is connected to W¹ or —CH(Y²)—when W¹ is a covalent bond. In embodiments, X¹ is —(*C═O)—O-optionally substituted (C₁₀-C₂₀)alkenyl, wherein the atom marked with a * is connected to W¹ or —CH(Y²)—when W¹ is a covalent bond. In embodiments, X¹ is —(*C═O)—O-optionally substituted (C₁₀-C₁₃)alkenyl, wherein the atom marked with a * is connected to W¹ or —CH(Y²)—when W¹ is a covalent bond. In embodiments, X¹ is —*O—(C═O)-optionally substituted (C₃-C₂₅)alkyl, wherein the atom marked with a * is connected to W¹ or —CH(Y²)—when W¹ is a covalent bond. In embodiments, X¹ is —*O—(C═O)-optionally substituted (C₃-C₂₀)alkyl, wherein the atom marked with a * is connected to W¹ or —CH(Y²)—when W¹ is a covalent bond. In embodiments, X¹ is —*O—(C═O)-optionally substituted (C₅-C₂₀)alkyl, wherein the atom marked with a * is connected to W¹ or —CH(Y²)—when W¹ is a covalent bond. In embodiments, X¹ is —*O—(C═O)-optionally substituted (C₅-C₁₃)alkyl, wherein the atom marked with a * is connected to W¹ or —CH(Y²)—when W¹ is a covalent bond. In embodiments, X¹ is —*O—(C═O)-optionally substituted (C₃-C₂₅)alkenyl, wherein the atom marked with a * is connected to W¹ or —CH(Y²)—when W¹ is a covalent bond. In embodiments, X¹ is —*O—(C═O)-optionally substituted (C₃-C₂₀)alkenyl, wherein the atom marked with a * is connected to W¹ or —CH(Y²)—when W¹ is a covalent bond. In embodiments, X¹ is —*O—(C═O)-optionally substituted (C₅-C₂₀)alkenyl, wherein the atom marked with a * is connected to W¹ or —CH(Y²)—when W¹ is a covalent bond. In embodiments, X¹ is —*O—(C═O)-optionally substituted (C₁₀-C₂₀)alkenyl, wherein the atom marked with a * is connected to W¹ or —CH(Y²)—when W¹ is a covalent bond. In embodiments, X¹ is —*O—(C═O)-optionally substituted (C₁₀-C₁₃)alkenyl, wherein the atom marked with a * is connected to W¹ or —CH(Y²)—when W¹ is a covalent bond.

In embodiments, R^{5D} is optionally substituted (C₅-C₂₅)alkyl. In embodiments, R^{5D} is optionally substituted (C₅-C₂₀)alkyl. In embodiments, R^{5D} is optionally substituted (C₅-C₁₅)alkyl. In embodiments, R^{5D} is optionally substituted (C₆-C₁₂)alkyl.

In embodiments, R^{5D} is optionally substituted (C₅-C₂₅)alkenyl. In embodiments, R^{5D} is optionally substituted (C₅-C₂₀)alkenyl. In embodiments, R^{5D} is optionally substituted (C₁₀-C₂₀)alkenyl. In embodiments, R^{5D} is optionally substituted (C₁₅-C₂₀)alkenyl.

In embodiments, R^{5D} is —W¹—X¹. In embodiments, W¹ is a covalent bond. In embodiments, W⁷ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y²)—when W¹ is a covalent bond. In embodiments, X¹ is —(*C═O)—O-optionally substituted (C₃-C₂₀)alkyl, wherein the atom marked with a * is connected to W¹ or —CH(Y²)—when W¹ is a covalent bond. In embodiments, X¹ is —(*C═O)—O-optionally substituted (C₅-C₂₀)alkyl, wherein the atom marked with a * is connected to W¹ or —CH(Y²)—when W¹ is a covalent bond. In embodiments, X¹ is —(*C═O)—O-optionally substituted (C₅-C₁₃)alkyl, wherein the atom marked with a * is connected to W¹ or —CH(Y²)—when W¹ is a covalent bond. In embodiments, X¹ is —(*C═O)—O-optionally substituted (C₃-C₂₅)alkenyl, wherein the atom marked with a * is connected to W¹ or —CH(Y²)—when W¹ is a covalent bond. In embodiments, X¹ is —(*C═O)—O-optionally substituted (C₃-C₂₀)alkenyl, wherein the atom marked with a * is connected to W¹ or —CH(Y²)—when W¹ is a covalent bond. In embodiments, X¹ is —(*C═O)—O-optionally substituted (C₅-C₂₀)alkenyl, wherein the atom marked with a * is connected to W¹ or —CH(Y²)—when W¹ is a covalent bond. In embodiments, X¹ is —(*C═O)—O-optionally substituted (C₁₀-C₂₀)alkenyl, wherein the atom marked with a * is connected to W¹ or —CH(Y²)—when W¹ is a covalent bond. In embodiments, X¹ is —(*C═O)—O-optionally substituted (C₁₀-C₁₃)alkenyl, wherein the atom marked with a * is connected to W¹ or —CH(Y²)—when W¹ is a covalent bond. In embodiments, X¹ is —*O—(C═O)-optionally substituted (C₃-C₂₅)alkyl, wherein the atom marked with a * is connected to W¹ or —CH(Y²)—when W¹ is a covalent bond. In embodiments, X¹ is —*O—(C═O)-optionally substituted (C₃-C₂₀)alkyl, wherein the atom marked with a * is connected to W¹ or —CH(Y²)—when W¹ is a covalent bond. In embodiments, X¹ is —*O—(C═O)-optionally substituted (C₅-C₂₀)alkyl, wherein the atom marked with a * is connected to W¹ or —CH(Y²)—when W¹ is a covalent bond. In embodiments, X¹ is —*O—(C═O)-optionally substituted (C₅-C₁₃)alkyl, wherein the atom marked with a * is connected to W¹ or —CH(Y²)—when W¹ is a covalent bond. In embodiments, X¹ is —*O—(C═O)-optionally substituted (C₃-C₂₅)alkenyl, wherein the atom marked with a * is connected to W¹ or —CH(Y²)—when W¹ is a covalent bond. In embodiments, X¹ is —*O—(C═O)-optionally substituted (C₃-C₂₀)alkenyl, wherein the atom marked with a * is connected to W¹ or —CH(Y²)—when W¹ is a covalent bond. In embodiments, X¹ is —*O—(C═O)-optionally substituted (C₅-C₂₀)alkenyl, wherein the atom marked with a * is connected to W¹ or —CH(Y²)—when W¹ is a covalent bond. In embodiments, X¹ is —*O—(C═O)-optionally substituted (C₁₀-C₂₀)alkenyl, wherein the atom marked with a * is connected to W¹ or —CH(Y²)—when W¹ is a covalent bond. In embodiments, X¹ is —*O—(C═O)-optionally substituted (C₁₀-C₁₃)alkenyl, wherein the atom marked with a * is connected to W¹ or —CH(Y²)—when W¹ is a covalent bond.

In embodiments, each R^{5A}, R^{5B}, R^{5C} and R^{5D} is independently selected from:

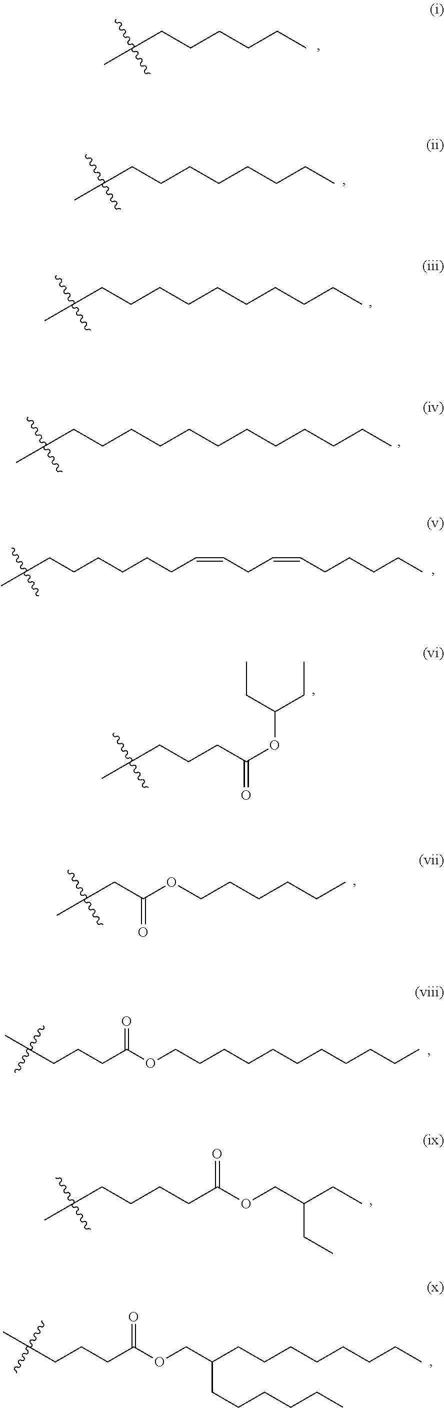

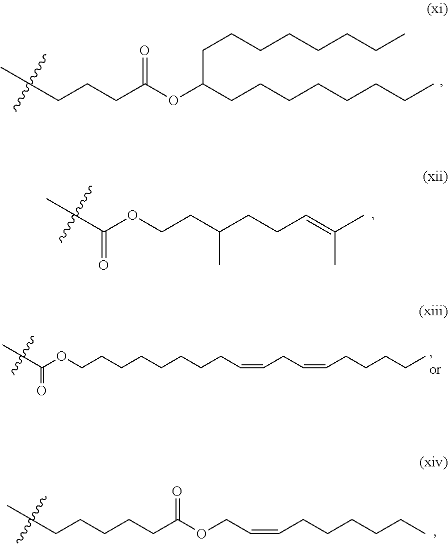 [SMILES: *CCCC(=O)OC(CCCCCCCC)CCCCCCCC ; *C(=O)OCCC(C)CCC=C(C)C ; *C(=O)OCCCCCCCC/C=C\C/C=C\CCCCC ; *CCCCCC(=O)OC/C=C\CCCCCC]

preferably wherein each R^{5A}, R^{5B}, R^{5C} and R^{5D} is independently selected from options (i), (ii), (iii), (vii), (viii), (xi) or (xiv).

In embodiments, each R^{5A}, R^{5B}, R^{5C} and R^{5D} is independently selected from:

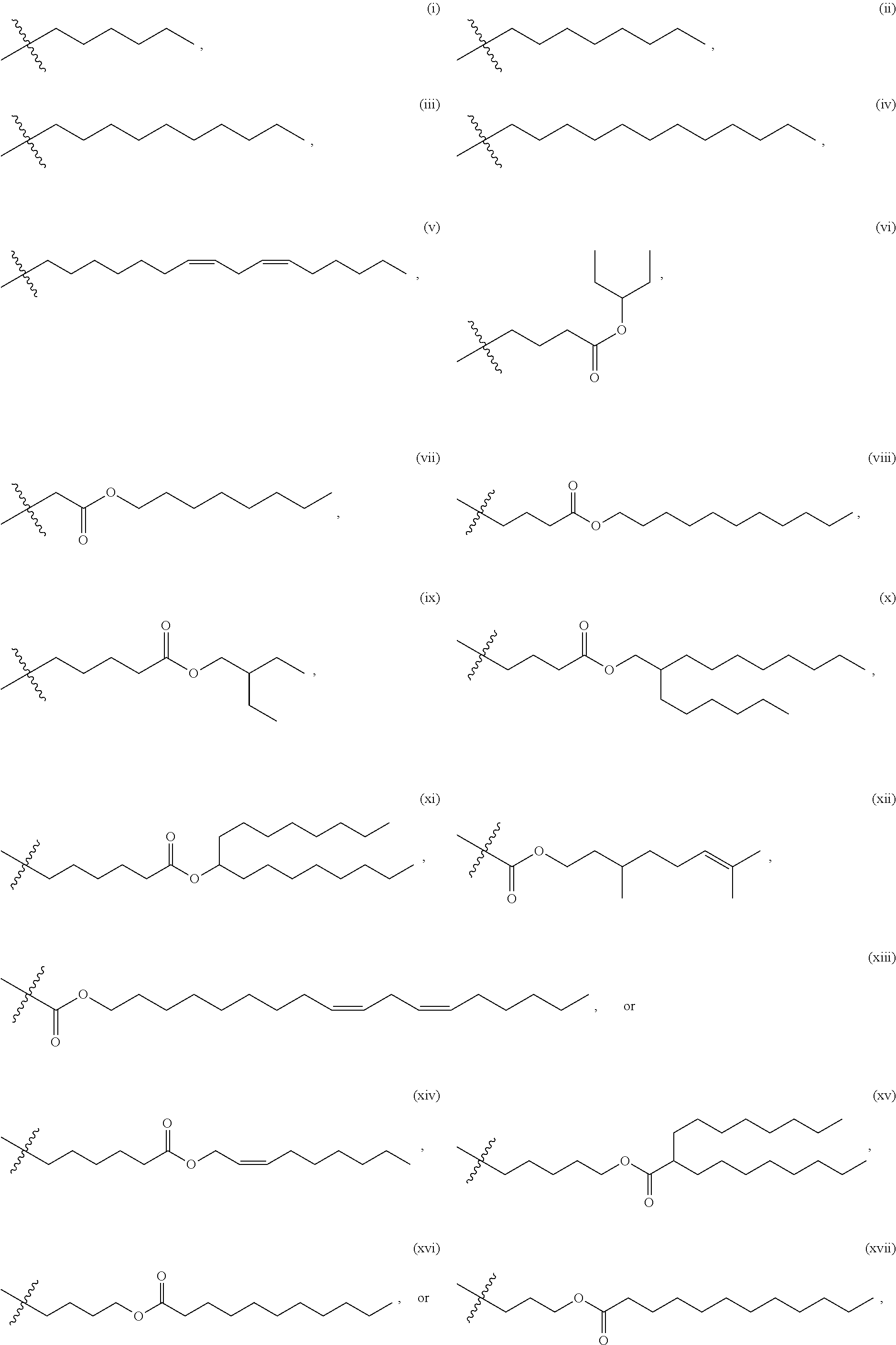

preferably wherein each R^{5A}, R^{5B}, R^{5C} and R^{5D} is independently selected from options (i), (ii), (iii), (vii), (viii), (xi) or (xiv).

In embodiments, R^{5A}, R^{5B}, R^{5C} and R^{5D} are the same. In embodiment, R^{5A} and R^{5B} are the same and R^{5C} and R^{5D} are the same. In embodiment, R^{5A} and R^{5C} are the same and R^{5B} and R^{5D} are the same.

The cationic lipids of the present invention also include compounds having a structure according to Formula (III):

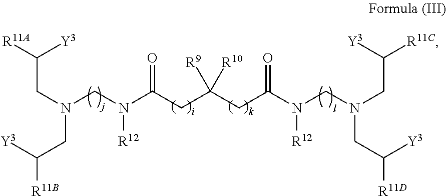

or a pharmaceutically acceptable salt thereof,
wherein R⁹ is selected from hydrogen, or optionally substituted (C₁-C₆)alkyl;
R¹⁰ is selected from hydrogen, —OH, —NH₂, optionally substituted (C₁-C₆)alkyl, optionally substituted aryl, optionally substituted heteroaryl, optionally substituted (C₁-C₃)alkylene-optionally substituted aryl, or optionally substituted (C₁-C₃)alkylene-optionally substituted heteroaryl;
i and k are integers that are each independently selected from 0, 1, 2, 3, or 4;
j and l are integers that are each independently selected from 1, 2, 3, 4, 5 or 6;
each Y³ is independently selected from hydrogen or —OH;
each R¹² is independently selected from hydrogen or optionally substituted (C₁-C₆)alkyl;
R^{11A}, R^{11B}, R^{11C}, and R^{11D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y³)—when W¹ is a covalent bond.

In embodiments, the cationic lipids of the present invention include compounds having a structure according to Formula (III) wherein Y³═OH. In embodiments, the cationic lipids of the present invention include compounds having a structure according to Formula (IIIA):

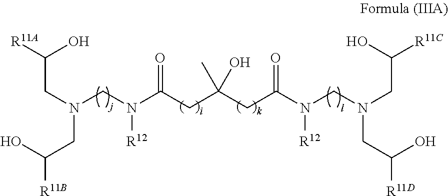

or a pharmaceutically acceptable salt thereof.

In embodiments, the cationic lipids of the present invention include compounds having a structure according to Formula (III) wherein Y³═OH, R⁹=methyl, and i and k=0.

In embodiments, the cationic lipids of the present invention include compounds having a structure according to Formula (III) wherein Y³═OH, R¹⁰═OH, and i and k=0. In embodiments, the cationic lipids of the present invention include compounds having a structure according to Formula (III) wherein Y³═OH, R⁹=methyl, R¹⁰═OH, and i and k=0.

In embodiments, the cationic lipids of the present invention include compounds having a structure according to Formula (IIIA1):

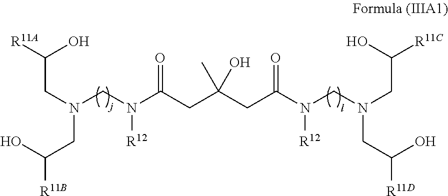

or a pharmaceutically acceptable salt thereof.

In embodiments, the cationic lipids of the present invention include compounds having a structure according to Formula (IIIB):

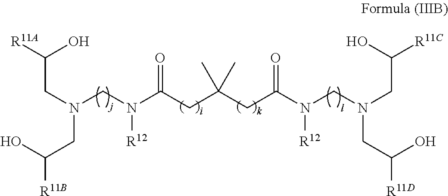

or a pharmaceutically acceptable salt thereof.

In embodiments, the cationic lipids of the present invention include compounds having a structure according to Formula (IIIB1):

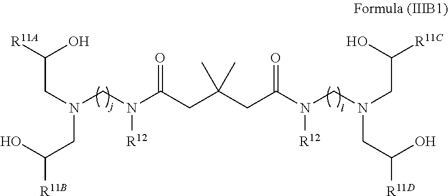

or a pharmaceutically acceptable salt thereof.

In embodiments, R⁹ is hydrogen. In embodiments, R⁹ is optionally substituted (C₁-C₆)alkyl. In embodiments, R⁹ is methyl.

In embodiments, R¹⁰ is hydrogen. In embodiments, R¹⁰ is —OH. In embodiments, R¹⁰ is —NH₂. In embodiments, R¹⁰ is optionally substituted (C₁-C₆)alkyl. In embodiments, R¹⁰ is methyl. In embodiments, R¹⁰ is ethyl. In embodiments, R¹⁰ is isopropyl. In embodiments, R¹⁰ is optionally substituted aryl. In embodiments, R¹⁰ is optionally substituted phenyl. In embodiments, R¹⁰ is phenyl. In embodiments, R¹⁰ is optionally substituted (C₁-C₃)alkylene-optionally substituted aryl. In embodiments, R¹⁰ is optionally substituted (C₁-C₃)alkylene-optionally substituted phenyl. In embodiments, R¹⁰ is optionally substituted benzyl. In embodiments, R¹⁰ is benzyl. In embodiments, R¹⁰ is optionally substituted heteroaryl. In embodiments, R¹⁰ is optionally substituted (C₁-C₃)alkylene-optionally substituted heteroaryl.

In embodiments, i is 0. In embodiments, i is 1. In embodiments, i is 2. In embodiments, i is 3. In embodiments, i is 4.

In embodiments, j is 3 or 4. In embodiments, j is 1. In embodiments, j is 2. In embodiments, j is 3, preferably wherein the cationic lipid has a structure according to any one of Formula (IIIB) or Formula (IIIB1). In embodiments, j is 4. In embodiments, j is 5. In embodiments, j is 6.

In embodiments, k is 0. In embodiments, k is 1. In embodiments, k is 2. In embodiments, k is 3. In embodiments, k is 4.

In embodiments, l is 3 or 4. In embodiments, l is 1. In embodiments, l is 2. In embodiments, l is 3, preferably wherein the cationic lipid has a structure according to any one of Formula (IIIB) or Formula (IIIB1). In embodiments, l is 4. In embodiments, l is 5. In embodiments, l is 6.

In embodiments, at least one Y³ is —OH. In embodiments, at least one Y³ is hydrogen. In embodiments, Y³ is —OH. In embodiments, Y³ is hydrogen.

In embodiments, each R¹² is hydrogen. In embodiments, each R¹² is optionally substituted (C₁-C₆)alkyl. In embodiments, each R¹² is methyl.

In embodiments, R^{11A} is optionally substituted (C₅-C₂₅)alkyl. In embodiments, R^{11A} is optionally substituted (C₅-C₂₀)alkyl. In embodiments, R^{11A} is optionally substituted (C₅-C₁₅)alkyl. In embodiments, R^{11A} is optionally substituted (C₆-C₁₂)alkyl. In embodiments, R^{11A} is optionally substituted (C₈-C₁₀)alkyl.

In embodiments, R^{11A} is optionally substituted (C₅-C₂₅)alkenyl. In embodiments, R^{11A} is optionally substituted (C₅-C₂₀)alkenyl. In embodiments, R^{11A} is optionally substituted (C₁₀-C₂₀)alkenyl. In embodiments, R^{11A} is optionally substituted (C₁₅-C₂₀)alkenyl.

In embodiments, R^{11A} is —W¹—X¹. In embodiments, W¹ is a covalent bond. In embodiments, W¹ is optionally substituted (C₁-C₁₀)alkylene. In embodiments, W¹ is optionally substituted (C₁-C₃)alkylene. In embodiments, W is optionally substituted (C₁-C₆)alkylene. In embodiments, W is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W⁷ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y³)—when W¹ is a covalent bond. In embodiments, X¹ is —(*C═O)—O-optionally substituted (C₃-C₂₀)alkyl, wherein the atom marked with a * is connected to W¹ or —CH(Y³)—when W¹ is a covalent bond. In embodiments, X¹ is —(*C═O)—O-optionally substituted (C₅-C₂₀)alkyl, wherein the atom marked with a * is connected to W¹ or —CH(Y³)—when W¹ is a covalent bond. In embodiments, X¹ is —(*C═O)—O-optionally substituted (C₅-C₁₃)alkyl, wherein the atom marked with a * is connected to W¹ or —CH(Y³)—when W¹ is a covalent bond. In embodiments, X¹ is —(*C═O)—O-optionally substituted (C₃-C₂₅)alkenyl, wherein the atom marked with a * is connected to W¹ or —CH(Y³)—when W¹ is a covalent bond. In embodiments, X¹ is —(*C═O)—O-optionally substituted (C₃-C₂₀)alkenyl, wherein the atom marked with a * is connected to W¹ or —CH(Y³)—when W¹ is a covalent bond. In embodiments, X¹ is —(*C═O)—O-optionally substituted (C₅-C₂₀)alkenyl, wherein the atom marked with a * is connected to W¹ or —CH(Y³)—when W¹ is a covalent bond. In embodiments, X¹ is —(*C═O)—O-optionally substituted (C₁₀-C₂₀)alkenyl, wherein the atom marked with a * is connected to W¹ or —CH(Y³)—when W¹ is a covalent bond. In embodiments, X¹ is —(*C═O)—O-optionally substituted (C₁₀-C₁₃)alkenyl, wherein the atom marked with a * is connected to W¹ or —CH(Y³)—when W¹ is a covalent bond. In embodiments, X¹ is —*O—(C═O)-optionally substituted (C₃-C₂₅)alkyl, wherein the atom marked with a * is connected to W¹ or —CH(Y³)—when W¹ is a covalent bond. In embodiments, X¹ is —*O—(C═O)-optionally substituted (C₃-C₂₀)alkyl, wherein the atom marked with a * is connected to W¹ or —CH(Y³)—when W¹ is a covalent bond. In embodiments, X¹ is —*O—(C═O)-optionally substituted (C₅-C₂₀)alkyl, wherein the atom marked with a * is connected to W¹ or —CH(Y³)—when W¹ is a covalent bond. In embodiments, X¹ is —*O—(C═O)-optionally substituted (C₅-C₁₃)alkyl, wherein the atom marked with a * is connected to W¹ or —CH(Y³)—when W¹ is a covalent bond. In embodiments, X¹ is —*O—(C═O)-optionally substituted (C₃-C₂₅)alkenyl, wherein the atom marked with a * is connected to W¹ or —CH(Y³)—when W¹ is a covalent bond. In embodiments, X¹ is —*O—(C═O)-optionally substituted (C₃-C₂₀)alkenyl, wherein the atom marked with a * is connected to W¹ or —CH(Y³)—when W¹ is a covalent bond. In embodiments, X¹ is —*O—(C═O)-optionally substituted (C₅-C₂₀)alkenyl, wherein the atom marked with a * is connected to W¹ or —CH(Y³)—when W¹ is a covalent bond. In embodiments, X¹ is —*O—(C═O)-optionally substituted (C₁₀-C₂₀)alkenyl, wherein the atom marked with a * is connected to W¹ or —CH(Y³)—when W¹ is a covalent bond. In embodiments, X¹ is —*O—(C═O)-optionally substituted (C₁₀-C₁₃)alkenyl, wherein the atom marked with a * is connected to W¹ or —CH(Y³)—when W¹ is a covalent bond.

In embodiments, R^{11B} is optionally substituted (C₅-C₂₅)alkyl. In embodiments, R^{11B} is optionally substituted (C₅-C₂₀)alkyl. In embodiments, R^{11B} is optionally substituted (C₅-C₁₅)alkyl. In embodiments, R^{1B} is optionally substituted (C₆-C₁₂)alkyl. In embodiments, R^{11B} is optionally substituted (C₈-C₁₀)alkyl.

In embodiments, R^{11B} is optionally substituted (C₅-C₂₅)alkenyl. In embodiments, R^{11B} is optionally substituted (C₅-C₂₀)alkenyl. In embodiments, R^{11B} is optionally substituted (C₁₀-C₂₀)alkenyl. In embodiments, R^{11B} is optionally substituted (C₁₅-C₂₀)alkenyl.

In embodiments, R^{11B} is —W¹—X¹. In embodiments, W¹ is a covalent bond. In embodiments, W¹ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W⁷ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y³)—when W¹ is a covalent bond. In embodiments, X¹ is —(*C═O)—O-optionally substituted (C₃-C₂₀)alkyl, wherein the atom marked with a * is connected to W¹ or —CH(Y³)—when W¹ is a covalent bond. In embodiments, X¹ is —(*C═O)—O-optionally substituted (C₅-C₂₀)alkyl, wherein the atom marked with a * is connected to W¹ or —CH(Y³)—when W¹ is a covalent bond. In embodiments, X¹ is —(*C═O)—O-optionally substituted (C₅-C₁₃)alkyl, wherein the atom marked with a * is connected to W¹ or —CH(Y³)—when W¹ is a covalent bond. In embodiments, X¹ is —(*C═O)—O-optionally substituted (C₃-C₂₅)alkenyl, wherein the atom marked with a * is connected to W¹ or —CH(Y³)—when W¹ is a covalent bond. In embodiments, X¹ is —(*C═O)—O-optionally substituted (C₃-C₂₀)alkenyl, wherein the atom marked with a * is connected to W¹ or —CH(Y³)—when W¹ is a covalent bond. In embodiments, X¹ is —(*C═O)—O-optionally substituted (C₅-C₂₀)alkenyl, wherein the atom marked with a * is connected to W¹ or —CH(Y³)—when W¹ is a covalent bond. In embodiments, X¹ is —(*C═O)—O-optionally substituted (C₁₀-C₂₀)alkenyl, wherein the atom marked with a * is connected to W¹ or —CH(Y³)—when W¹ is a covalent bond. In embodiments, X¹ is —(*C═O)—O-optionally substituted (C₁₀-C₁₃)alkenyl, wherein the atom marked with a * is connected to W¹ or —CH(Y³)—when W¹ is a covalent bond. In embodiments, X¹ is —*O—(C═O)-optionally substituted (C₃-C₂₅)alkyl, wherein the atom marked with a * is connected to W¹ or —CH(Y³)—when W¹ is a covalent bond. In embodiments, X¹ is —*O—(C═O)-optionally substituted (C₃-C₂₀)alkyl, wherein the atom marked with a * is connected to W¹ or —CH(Y³)—when W¹ is a covalent bond. In embodiments, X¹ is —*O—(C═O)-optionally substituted (C₅-C₂₀)alkyl, wherein the atom marked with a * is connected to W¹ or —CH(Y³)—when W¹ is a covalent bond. In embodiments, X¹ is —*O—(C═O)-optionally substituted (C₅-C₁₃)alkyl, wherein the atom marked with a * is connected to W¹ or —CH(Y³)—when W¹ is a covalent bond. In embodiments, X¹ is —*O—(C═O)-optionally substituted (C₃-C₂₅)alkenyl, wherein the atom marked with a * is connected to W¹ or —CH(Y³)—when W¹ is a covalent bond. In embodiments, X¹ is —*O—(C═O)-optionally substituted (C₃-C₂₀)alkenyl, wherein the atom marked with a * is connected to W¹ or —CH(Y³)—when W¹ is a covalent bond. In embodiments, X¹ is —*O—(C═O)-optionally substituted (C₅-C₂₀)alkenyl, wherein the atom marked with a * is connected to W¹ or —CH(Y³)—when W¹ is a covalent bond. In embodiments, X¹ is —*O—(C═O)-optionally substituted (C₁₀-C₂₀)alkenyl, wherein the atom marked with a * is connected to W¹ or —CH(Y³)—when W¹ is a covalent bond. In embodiments, X¹ is —*O—(C═O)-optionally substituted (C₁₀-C₁₃)alkenyl, wherein the atom marked with a * is connected to W¹ or —CH(Y³)—when W¹ is a covalent bond.

In embodiments, R^{11C} is optionally substituted (C₅-C₂₅)alkyl. In embodiments, R^{11C} is optionally substituted (C₅-C₂₀)alkyl. In embodiments, R^{11C} is optionally substituted (C₅-C₁₅)alkyl. In embodiments, R^{11C} is optionally substituted (C₆-C₁₂)alkyl. In embodiments, R^{11C} is optionally substituted (C₃-C₁₀)alkyl.

In embodiments, R^{11C} is optionally substituted (C₅-C₂₅)alkenyl. In embodiments, R^{11C} is optionally substituted (C₅-C₂₀)alkenyl. In embodiments, R^{11C} is optionally substituted (C₁₀-C₂₀)alkenyl. In embodiments, R^{11C} is optionally substituted (C₁₅-C₂₀)alkenyl.

In embodiments, R^{11C} is —W¹—X¹. In embodiments, W¹ is a covalent bond. In embodiments, W⁷ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W¹ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y³)—when W¹ is a covalent bond. In embodiments, X¹ is —(*C═O)—O-optionally substituted (C₃-C₂₀)alkyl, wherein the atom marked with a * is connected to W¹ or —CH(Y³)—when W¹ is a covalent bond. In embodiments, X¹ is —(*C═O)—O-optionally substituted (C₅-C₂₀)alkyl, wherein the atom marked with a * is connected to W¹ or —CH(Y³)—when W¹ is a covalent bond. In embodiments, X¹ is —(*C═O)—O-optionally substituted (C₅-C₁₃)alkyl, wherein the atom marked with a * is connected to W¹ or —CH(Y³)—when W¹ is a covalent bond. In embodiments, X¹ is —(*C═O)—O-optionally substituted (C₃-C₂₅)alkenyl, wherein the atom marked with a * is connected to W¹ or —CH(Y³)—when W¹ is a covalent bond. In embodiments, X¹ is —(*C═O)—O-optionally substituted (C₃-C₂₀)alkenyl, wherein the atom marked with a * is connected to W¹ or —CH(Y³)—when W¹ is a covalent bond. In embodiments, X¹ is —(*C═O)—O-optionally substituted (C₅-C₂₀)alkenyl, wherein the atom marked with a * is connected to W¹ or —CH(Y³)—when W¹ is a covalent bond. In embodiments, X¹ is —(*C═O)—O-optionally substituted (C₁₀-C₂₀)alkenyl, wherein the atom marked with a * is connected to W¹ or —CH(Y³)—when W¹ is a covalent bond. In embodiments, X¹ is —(*C═O)—O-optionally substituted (C₁₀-C₁₃)alkenyl, wherein the atom marked with a * is connected to W¹ or —CH(Y³)—when W¹ is a covalent bond. In embodiments, X¹ is —*O—(C═O)-optionally substituted (C₃-C₂₅)alkyl, wherein the atom marked with a * is connected to W¹ or —CH(Y³)—when W¹ is a covalent bond. In embodiments, X¹ is —*O—(C═O)-optionally substituted (C₃-C₂₀)alkyl, wherein the atom marked with a * is connected to W¹ or —CH(Y³)—when W¹ is a covalent bond. In embodiments, X¹ is —*O—(C═O)-optionally substituted (C₅-C₂₀)alkyl, wherein the atom marked with a * is connected to W¹ or —CH(Y³)—when W¹ is a covalent bond. In embodiments, X¹ is —*O—(C═O)-optionally substituted (C₅-C₁₃)alkyl, wherein the atom marked with a * is connected to W¹ or —CH(Y³)—when W¹ is a covalent bond. In embodiments, X¹ is —*O—(C═O)-optionally substituted (C₃-C₂₅)alkenyl, wherein the atom marked with a * is connected to W¹ or —CH(Y³)—when W¹ is a covalent bond. In embodiments, X¹ is —*O—(C═O)-optionally substituted (C₃-C₂₀)alkenyl, wherein the atom marked with a * is connected to W¹ or —CH(Y³)—when W¹ is a covalent bond. In embodiments, X¹ is —*O—(C═O)-optionally substituted (C₅-C₂₀)alkenyl, wherein the atom marked with a * is connected to W¹ or —CH(Y³)—when W¹ is a covalent bond. In embodiments, X¹ is —*O—(C═O)-optionally substituted (C₁₀-C₂₀)alkenyl, wherein the atom marked with a * is connected to W¹ or —CH(Y³)—when W¹ is a covalent bond. In embodiments, X¹ is —*O—(C═O)-optionally substituted (C₁₀-C₁₃)alkenyl, wherein the atom marked with a * is connected to W¹ or —CH(Y³)—when W¹ is a covalent bond.

In embodiments, R^{11D} is optionally substituted (C₅-C₂₅)alkyl. In embodiments, R^{11D} is optionally substituted (C₅-C₂₀)alkyl. In embodiments, R^{11D} is optionally substituted (C₅-C₁₅)alkyl. In embodiments, R^{11D} is optionally substituted (C₆-C₁₂)alkyl. In embodiments, R^{11D} is optionally substituted (C₃-C₁₀)alkyl.

In embodiments, R^{11D} is optionally substituted (C₅-C₂₅)alkenyl. In embodiments, R^{11D} is optionally substituted (C₅-C₂₀)alkenyl. In embodiments, R^{11D} is optionally substituted (C₁₀-C₂₀)alkenyl. In embodiments, R^{11D} is optionally substituted (C₁₅-C₂₀)alkenyl.

In embodiments, R^{11D} is —W¹—X¹. In embodiments, W¹ is a covalent bond. In embodiments, W⁷ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y³)—when W¹ is a covalent bond. In embodiments, X¹ is —(*C═O)—O-optionally substituted (C₃-C₂₀)alkyl, wherein the atom marked with a * is connected to W¹ or —CH(Y³)—when W¹ is a covalent bond. In embodiments, X¹ is —(*C═O)—O-optionally substituted (C₅-C₂₀)alkyl, wherein the atom marked with a * is connected to W¹ or —CH(Y³)—when W¹ is a covalent bond. In embodiments, X¹ is —(*C═O)—O-optionally substituted (C₅-C₁₃)alkyl, wherein the atom marked with a * is connected to W¹ or —CH(Y³)—when W¹ is a covalent bond. In embodiments, X¹ is —(*C═O)—O-optionally substituted (C₃-C₂₅)alkenyl, wherein the atom marked with a * is connected to W¹ or —CH(Y³)—when W¹ is a covalent bond. In embodiments, X¹ is —(*C═O)—O-optionally substituted (C₃-C₂₀)alkenyl, wherein the atom marked with a * is connected to W¹ or —CH(Y³)—when W¹ is a covalent bond. In embodiments, X¹ is —(*C═O)—O-optionally substituted (C₅-C₂₀)alkenyl, wherein the atom marked with a * is connected to W¹ or —CH(Y³)—when W¹ is a covalent bond. In embodiments, X¹ is —(*C═O)—O-optionally substituted (C₁₀-C₂₀)alkenyl, wherein the atom marked with a * is connected to W¹ or —CH(Y³)—when W¹ is a covalent bond. In embodiments, X¹ is —(*C═O)—O-optionally substituted (C₁₀-C₁₃)alkenyl, wherein the atom marked with a * is connected to W¹ or —CH(Y³)—when W¹ is a covalent bond. In embodiments, X¹ is —*O—(C═O)-optionally substituted (C₃-C₂₅)alkyl, wherein the atom marked with a * is connected to W¹ or —CH(Y³)—when W¹ is a covalent bond. In embodiments, X¹ is —*O—(C═O)-optionally substituted (C₃-C₂₀)alkyl, wherein the atom marked with a * is connected to W¹ or —CH(Y³)—when W¹ is a covalent bond. In embodiments, X¹ is —*O—(C═O)-optionally substituted (C₅-C₂₀)alkyl, wherein the atom marked with a * is connected to W¹ or —CH(Y³)—when W¹ is a covalent bond. In embodiments, X¹ is —*O—(C═O)-optionally substituted (C₅-C₁₃)alkyl, wherein the atom marked with a * is connected to W¹ or —CH(Y³)—when W¹ is a covalent bond. In embodiments, X¹ is —*O—(C═O)-optionally substituted (C₃-C₂₅)alkenyl, wherein the atom marked with a * is connected to W¹ or —CH(Y³)—when W¹ is a covalent bond. In embodiments, X¹ is —*O—(C═O)-optionally substituted (C₃-C₂₀)alkenyl, wherein the atom marked with a * is connected to W¹ or —CH(Y³)—when W¹ is a covalent bond. In embodiments, X¹ is —*O—(C═O)-optionally substituted (C₅-C₂₀)alkenyl, wherein the atom marked with a * is connected to W¹ or —CH(Y³)—when W¹ is a covalent bond. In embodiments, X¹ is —*O—(C═O)-optionally substituted (C₁₀-C₂₀)alkenyl, wherein the atom marked with a * is connected to W¹ or —CH(Y³)—when W¹ is a covalent bond. In embodiments, X¹ is —*O—(C═O)-optionally substituted (C₁₀-C₁₃)alkenyl, wherein the atom marked with a * is connected to W¹ or —CH(Y³)—when W¹ is a covalent bond.

In embodiments, each R^{11A}, R^{11B}, R^{11C} and R^{11D} is independently selected from:

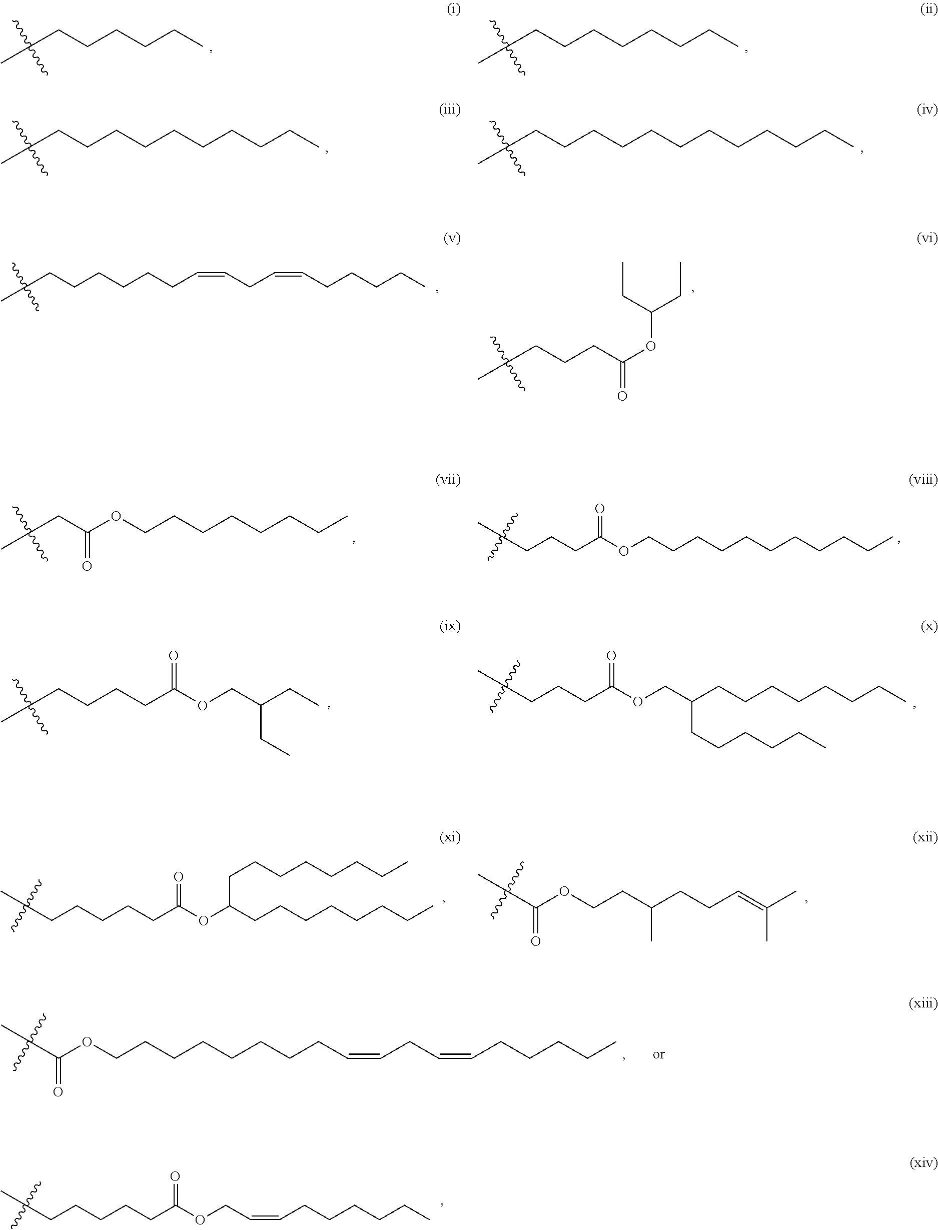

preferably wherein each R^{11A}, R^{11B}, R^{11C} and R^{11D} is independently selected from options (ii) or (iii).

In embodiments, each R^{11A}, R^{11B}, R^{11C} and R^{11D} is independently selected from:

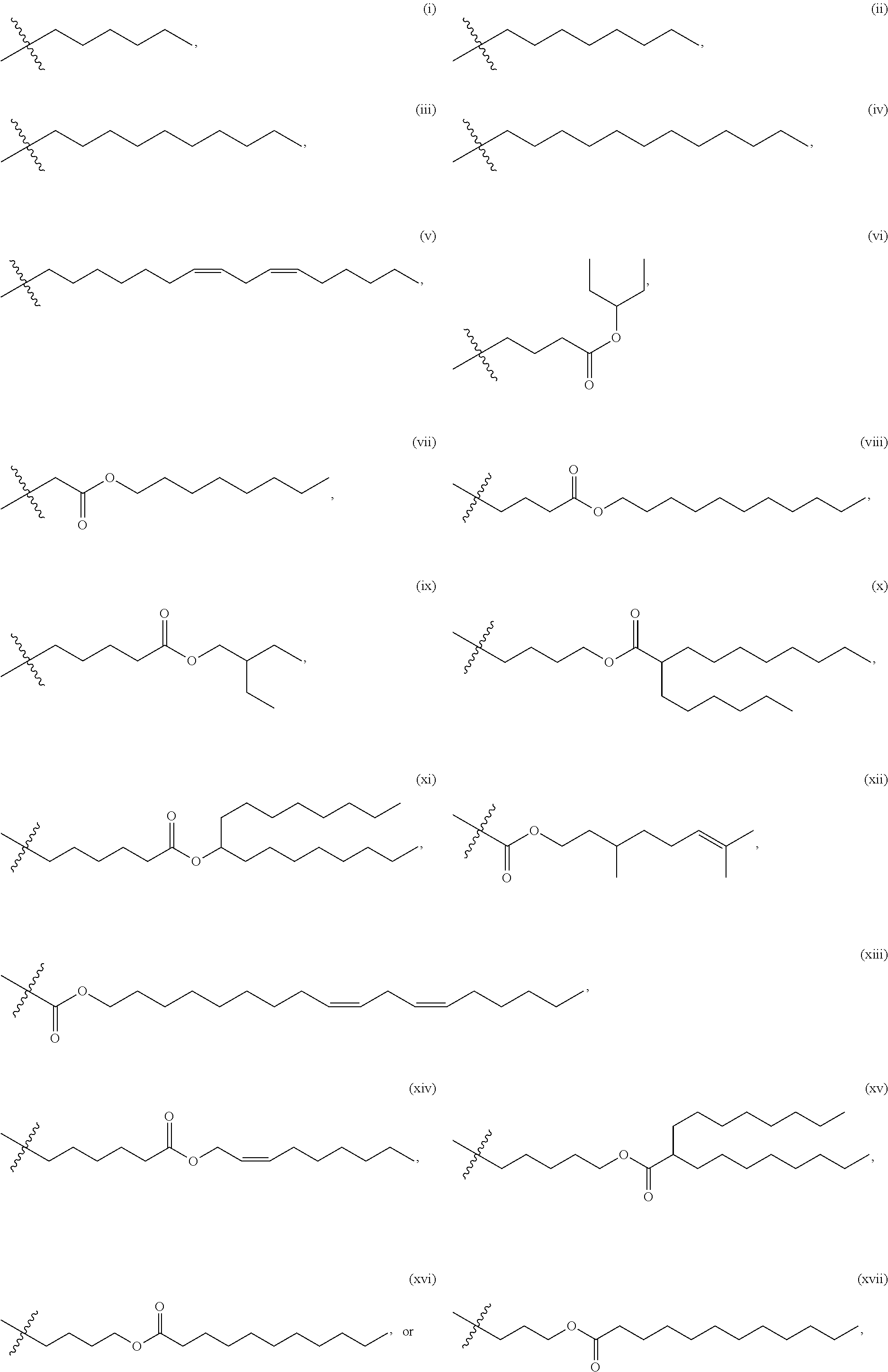

preferably wherein each R^{11A}, R^{11B}, R^{11C} and R^{11D} is independently selected from options (ii), (iii), (viii), (xi), (xiv), (xv), (xvi) or (xvii).

In embodiments, R^{11A}, R^{11B}, R^{11C} and R^{11D} are the same. In embodiments, R^{11A} and R^{11C} are the same and R^{11B} and R^{11D} are the same. In embodiments, R^{11A} and R^{11C} are the same and R^{11B} and R^{11D} are different.

The cationic lipids of the present invention also include compounds having a structure according to Formula (IV):

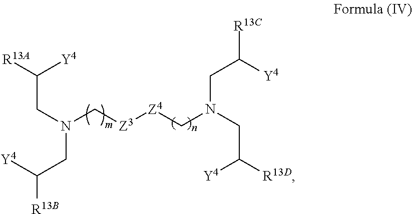

or a pharmaceutically acceptable salt thereof,
wherein
   m and n are integers that are each independently selected from 1, 2, 3, 4, 5 or 6;
   Z³ is an aromatic amino acid residue, wherein the α-carbon carboxyl group (—C(O)O—) of the aromatic amino acid residue is bound to the —(CH₂)ₘ and the α-carbon aminyl group (—NH—) of the aromatic amino acid residue is bound to the Z⁴;
Z⁴ is selected from

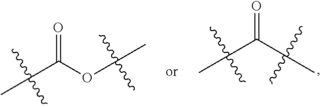

wherein the right hand side of each depicted structure is bound to the —(CH₂)ₙ—;
each Y⁴ is independently selected from hydrogen or —OH;
R^{13A}, R^{13B}, R^{13C}, and R^{13D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y⁴)—when W¹ is a covalent bond.

In embodiments, the cationic lipids of the present invention include compounds having a structure according to Formula (IV) wherein Y⁴═OH. In embodiments, the cationic lipids of the present invention include compounds having a structure according to Formula (IVA):

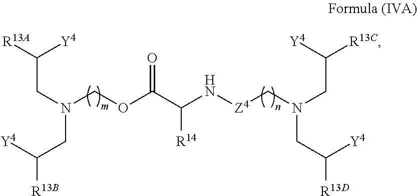

wherein R¹⁴ is optionally substituted (C₁-C₆)-alkylene-R¹⁵; and
R¹⁵ is selected from optionally substituted aryl or optionally substituted heteroaryl
or a pharmaceutically acceptable salt thereof.

In embodiments, the cationic lipids of the present invention include compounds having a structure according to Formula (IVA) wherein Y⁴═OH. In embodiments, the cationic lipids of the present invention include compounds having a structure according to Formula (IVA1):

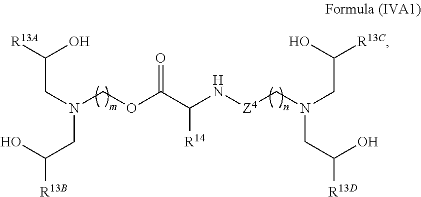

wherein R¹⁴ is optionally substituted (C₁-C₆)-alkylene-R¹⁵; and
R¹⁵ is selected from optionally substituted aryl or optionally substituted heteroaryl
or a pharmaceutically acceptable salt thereof.

In embodiments, the cationic lipids of the present invention include compounds having a structure according to Formula (IVA2):

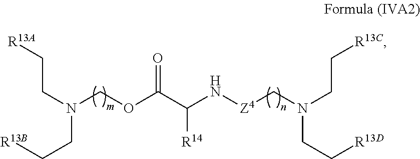

or a pharmaceutically acceptable salt thereof
wherein R¹⁴ is optionally substituted (C₁-C₆)-alkylene-R¹⁵; and
R¹⁵ is selected from optionally substituted aryl or optionally substituted heteroaryl.

In embodiments, m is 1. In embodiments, m is 2. In embodiments, m is 3. In embodiments, m is 4. In embodiments, m is 5. In embodiments, m is 6.

In embodiments, n is 4 or 5. In embodiments, n is 1. In embodiments, n is 2. In embodiments, n is 3. In embodiments, n is 4. In embodiments, n is 5. In embodiments, n is 6.

In embodiments, Z⁴ is

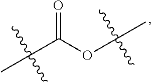 [SMILES: *OC(*)=O]

wherein the right hand side of the depicted structure is bound to the —(CH₂)ₙ—. In embodiments, Z⁴ is

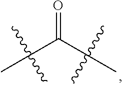 [SMILES: *C(*)=O]

wherein the right hand side of the depicted structure is bound to the —(CH₂)ₙ—.

In embodiments, at least one Y⁴ is —OH. In embodiments, at least one Y⁴ is hydrogen. In embodiments, Y⁴ is —OH. In embodiments, Y⁴ is hydrogen.

In embodiments, R¹⁴ is optionally substituted (C₁-C₆)-alkylene-R¹⁵. In embodiments, R¹⁵ is optionally substituted aryl. In embodiments, R¹⁵ is optionally substituted heteroaryl.

In embodiments, R¹⁴ is —(CH₂)-optionally substituted aryl. In embodiments, R¹⁴ is —(CH₂)₂-optionally substituted aryl. In embodiments, R¹⁴ is —(CH₂)-optionally substituted heteroaryl. In embodiments, R¹⁴ is —(CH₂)₂-optionally substituted heteroaryl. In embodiments, R¹⁴ is —(CH₂)-optionally substituted phenyl. In embodiments, R¹⁴ is —(CH₂)₂-optionally substituted phenyl. In embodiments, R¹⁴ is —(CH₂)-optionally substituted imidazolyl. In embodiments, R¹⁴ is —(CH₂)₂-optionally substituted imidazolyl. In embodiments, R¹⁴ is —(CH₂)-optionally substituted indolyl. In embodiments, R¹⁴ is

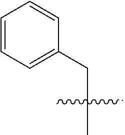 [SMILES: *Cc1ccccc1]

In embodiments, R¹⁴ is

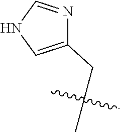 [SMILES: *Cc1c[nH]cn1]

In embodiments, R¹⁴ is

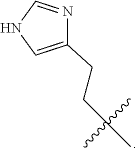 [SMILES: *CCc1c[nH]cn1]

In embodiments, R¹⁴ is

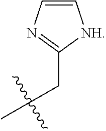 [SMILES: *Cc1ncc[nH]1]

In embodiments, R¹⁴ is

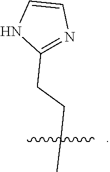 [SMILES: *CCc1ncc[nH]1]

In embodiments, R¹⁴ is

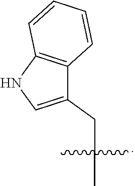 [SMILES: *Cc1c[nH]c2ccccc12]

In embodiments, R¹⁴ is

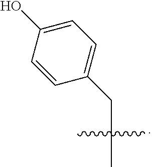 [SMILES: *Cc1ccc(O)cc1]

In embodiments, R¹⁴ is

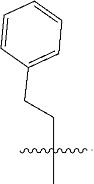 [SMILES: *CCc1ccccc1]

In embodiments, R¹⁴ is

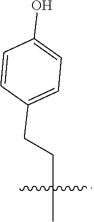 [SMILES: *CCc1ccc(O)cc1]

In embodiments, R¹⁴ is

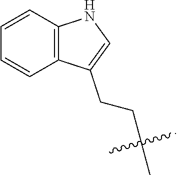 [SMILES: *CCc1c[nH]c2ccccc12]

In embodiments, R¹⁴ is

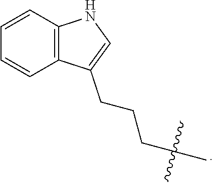 [SMILES: *CCCc1c[nH]c2ccccc12]

In embodiments, R^{13A} is optionally substituted (C₅-C₂₅)alkyl. In embodiments, R^{13A} is optionally substituted (C₅-C₂₀)alkyl. In embodiments, R^{13A} is optionally substituted (C₅-C₁₅)alkyl. In embodiments, R^{13A} is optionally substituted (C₆-C₁₂)alkyl. In embodiments, R^{13A} is optionally substituted (C₈-C₁₀)alkyl.

In embodiments, R^{13A} is optionally substituted (C₅-C₂₅)alkenyl. In embodiments, R^{13A} is optionally substituted (C₅-C₂₀)alkenyl. In embodiments, R^{13A} is optionally substituted (C₁₀-C₂₀)alkenyl. In embodiments, R^{13A} is optionally substituted (C₁₅-C₂₀)alkenyl.

In embodiments, R^{13A} is —W¹—X¹. In embodiments, W¹ is a covalent bond. In embodiments, W¹ is optionally substituted (C₁-C₁₀)alkylene. In embodiments, W¹ is optionally substituted (C₁-C₃)alkylene. In embodiments, W is optionally substituted (C₁-C₆)alkylene. In embodiments, W¹ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y⁴)—when W¹ is a covalent bond. In embodiments, X¹ is —(*C═O)—O-optionally substituted (C₃-C₂₀)alkyl, wherein the atom marked with a * is connected to W¹ or —CH(Y⁴)—when W¹ is a covalent bond. In embodiments, X¹ is —(*C═O)—O-optionally substituted (C₅-C₂₀)alkyl, wherein the atom marked with a * is connected to W¹ or —CH(Y⁴)—when W¹ is a covalent bond. In embodiments, X¹ is —(*C═O)—O-optionally substituted (C₅-C₁₃)alkyl, wherein the atom marked with a * is connected to W¹ or —CH(Y⁴)—when W¹ is a covalent bond. In embodiments, X¹ is —(*C═O)—O-optionally substituted (C₃-C₂₅)alkenyl, wherein the atom marked with a * is connected to W¹ or —CH(Y⁴)—when W¹ is a covalent bond. In embodiments, X¹ is —(*C═O)—O-optionally substituted (C₃-C₂₀)alkenyl, wherein the atom marked with a * is connected to W¹ or —CH(Y⁴)—when W¹ is a covalent bond. In embodiments, X¹ is —(*C═O)—O-optionally substituted (C₅-C₂₀)alkenyl, wherein the atom marked with a * is connected to W¹ or —CH(Y⁴)—when W¹ is a covalent bond. In embodiments, X¹ is —(*C═O)—O-optionally substituted (C₁₀-C₂₀)alkenyl, wherein the atom marked with a * is connected to W¹ or —CH(Y⁴)—when W¹ is a covalent bond. In embodiments, X¹ is —(*C═O)—O-optionally substituted (C₁₀-C₁₃)alkenyl, wherein the atom marked with a * is connected to W¹ or —CH(Y⁴)—when W¹ is a covalent bond. In embodiments, X¹ is —*O—(C═O)-optionally substituted (C₃-C₂₅)alkyl, wherein the atom marked with a * is connected to W¹ or —CH(Y⁴)—when W¹ is a covalent bond. In embodiments, X¹ is —*O—(C═O)-optionally substituted (C₃-C₂₀)alkyl, wherein the atom marked with a * is connected to W¹ or —CH(Y⁴)—when W¹ is a covalent bond. In embodiments, X¹ is —*O—(C═O)-optionally substituted (C₅-C₂₀)alkyl, wherein the atom marked with a * is connected to W¹ or —CH(Y⁴)—when W¹ is a covalent bond. In embodiments, X¹ is —*O—(C═O)-optionally substituted (C₅-C₁₃)alkyl, wherein the atom marked with a * is connected to W¹ or —CH(Y⁴)—when W¹ is a covalent bond. In embodiments, X¹ is —*O—(C═O)-optionally substituted (C₃-C₂₅)alkenyl, wherein the atom marked with a * is connected to W¹ or —CH(Y⁴)—when W¹ is a covalent bond. In embodiments, X¹ is —*O—(C═O)-optionally substituted (C₃-C₂₀)alkenyl, wherein the atom marked with a * is connected to W¹ or —CH(Y⁴)—when W¹ is a covalent bond. In embodiments, X¹ is —*O—(C═O)-optionally substituted (C₅-C₂₀)alkenyl, wherein the atom marked with a * is connected to W¹ or —CH(Y⁴)—when W¹ is a covalent bond. In embodiments, X¹ is —*O—(C═O)-optionally substituted (C₁₀-C₂₀)alkenyl, wherein the atom marked with a * is connected to W¹ or —CH(Y⁴)—when W¹ is a covalent bond. In embodiments, X¹ is —*O—(C═O)-optionally substituted (C₁₀-C₁₃)alkenyl, wherein the atom marked with a * is connected to W¹ or —CH(Y⁴)—when W¹ is a covalent bond.

In embodiments, R^{13B} is optionally substituted (C₅-C₂₅)alkyl. In embodiments, R^{13B} is optionally substituted (C₅-C₂₀)alkyl. In embodiments, R^{13B} is optionally substituted (C₅-C₁₅)alkyl. In embodiments, R^{13B} is optionally substituted (C₆-C₁₂)alkyl. In embodiments, R^{13B} is optionally substituted (C₈-C₁₀)alkyl.

In embodiments, R^{13B} is optionally substituted (C₅-C₂₅)alkenyl. In embodiments, R^{13B} is optionally substituted (C₅-C₂₀)alkenyl. In embodiments, R^{13B} is optionally substituted (C₁₀-C₂₀)alkenyl. In embodiments, R^{13B} is optionally substituted (C₁₅-C₂₀)alkenyl.

In embodiments, R^{13B} is —W¹—X¹. In embodiments, W¹ is a covalent bond. In embodiments, W⁷ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W⁷ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y⁴)—when W¹ is a covalent bond. In embodiments, X¹ is —(*C═O)—O-optionally substituted (C₃-C₂₀)alkyl, wherein the atom marked with a * is connected to W¹ or —CH(Y⁴)—when W¹ is a covalent bond. In embodiments, X¹ is —(*C═O)—O-optionally substituted (C₅-C₂₀)alkyl, wherein the atom marked with a * is connected to W¹ or —CH(Y⁴)—when W¹ is a covalent bond. In embodiments, X¹ is —(*C═O)—O-optionally substituted (C₅-C₁₃)alkyl, wherein the atom marked with a * is connected to W¹ or —CH(Y⁴)—when W¹ is a covalent bond. In embodiments, X¹ is —(*C═O)—O-optionally substituted (C₃-C₂₅)alkenyl, wherein the atom marked with a * is connected to W¹ or —CH(Y⁴)—when W¹ is a covalent bond. In embodiments, X¹ is —(*C═O)—O-optionally substituted (C₃-C₂₀)alkenyl, wherein the atom marked with a * is connected to W¹ or —CH(Y⁴)—when W¹ is a covalent bond. In embodiments, X¹ is —(*C═O)—O-optionally substituted (C₅-C₂₀)alkenyl, wherein the atom marked with a * is connected to W¹ or —CH(Y⁴)—when W¹ is a covalent bond. In embodiments, X¹ is —(*C═O)—O-optionally substituted (C₁₀-C₂₀)alkenyl, wherein the atom marked with a * is connected to W¹ or —CH(Y⁴)—when W¹ is a covalent bond. In embodiments, X¹ is —(*C═O)—O-optionally substituted (C₁₀-C₁₃)alkenyl, wherein the atom marked with a * is connected to W¹ or —CH(Y⁴)—when W¹ is a covalent bond. In embodiments, X¹ is —*O—(C═O)-optionally substituted (C₃-C₂₅)alkyl, wherein the atom marked with a * is connected to W¹ or —CH(Y⁴)—when W¹ is a covalent bond. In embodiments, X¹ is —*O—(C═O)-optionally substituted (C₃-C₂₀)alkyl, wherein the atom marked with a * is connected to W¹ or —CH(Y⁴)—when W¹ is a covalent bond. In embodiments, X¹ is —*O—(C═O)-optionally substituted (C₅-C₂₀)alkyl, wherein the atom marked with a * is connected to W¹ or —CH(Y⁴)—when W¹ is a covalent bond. In embodiments, X¹ is —*O—(C═O)-optionally substituted (C₅-C₁₃)alkyl, wherein the atom marked with a * is connected to W¹ or —CH(Y⁴)—when W¹ is a covalent bond. In embodiments, X¹ is —*O—(C═O)-optionally substituted (C₃-C₂₅)alkenyl, wherein the atom marked with a * is connected to W¹ or —CH(Y⁴)—when W¹ is a covalent bond. In embodiments, X¹ is —*O—(C═O)-optionally substituted (C₃-C₂₀)alkenyl, wherein the atom marked with a * is connected to W¹ or —CH(Y⁴)—when W¹ is a covalent bond. In embodiments, X¹ is —*O—(C═O)-optionally substituted (C₅-C₂₀)alkenyl, wherein the atom marked with a * is connected to W¹ or —CH(Y⁴)—when W¹ is a covalent bond. In embodiments, X¹ is —*O—(C═O)-optionally substituted (C₁₀-C₂₀)alkenyl, wherein the atom marked with a * is connected to W¹ or —CH(Y⁴)—when W¹ is a covalent bond. In embodiments, X¹ is —*O—(C═O)-optionally substituted (C₁₀-C₁₃)alkenyl, wherein the atom marked with a * is connected to W¹ or —CH(Y⁴)—when W¹ is a covalent bond.

In embodiments, R^{13C} is optionally substituted (C₅-C₂₅)alkyl. In embodiments, R^{13C} is optionally substituted (C₅-C₂₀)alkyl. In embodiments, R^{13C} is optionally substituted (C₅-C₁₅)alkyl. In embodiments, R^{13C} is optionally substituted (C₆-C₁₂)alkyl. In embodiments, R^{13C} is optionally substituted (C₃-C₁₀)alkyl.

In embodiments, R^{13C} is optionally substituted (C₅-C₂₅)alkenyl. In embodiments, R^{13C} is optionally substituted (C₅-C₂₀)alkenyl. In embodiments, R^{13C} is optionally substituted (C₁₀-C₂₀)alkenyl. In embodiments, R^{13C} is optionally substituted (C₁₅-C₂₀)alkenyl.

In embodiments, R^{13C} is —W¹—X¹. In embodiments, W¹ is a covalent bond. In embodiments, W⁷ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W⁷ is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W¹ is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y⁴)—when W¹ is a covalent bond. In embodiments, X¹ is —(*C═O)—O-optionally substituted (C₃-C₂₀)alkyl, wherein the atom marked with a * is connected to W¹ or —CH(Y⁴)—when W¹ is a covalent bond. In embodiments, X¹ is —(*C═O)—O-optionally substituted (C₅-C₂₀)alkyl, wherein the atom marked with a * is connected to W¹ or —CH(Y⁴)—when W¹ is a covalent bond. In embodiments, X¹ is —(*C═O)—O-optionally substituted (C₅-C₁₃)alkyl, wherein the atom marked with a * is connected to W¹ or —CH(Y⁴)—when W¹ is a covalent bond. In embodiments, X¹ is —(*C═O)—O-optionally substituted (C₃-C₂₅)alkenyl, wherein the atom marked with a * is connected to W¹ or —CH(Y⁴)—when W¹ is a covalent bond. In embodiments, X¹ is —(*C═O)—O-optionally substituted (C₃-C₂₀)alkenyl, wherein the atom marked with a * is connected to W¹ or —CH(Y⁴)—when W¹ is a covalent bond. In embodiments, X¹ is —(*C═O)—O-optionally substituted (C₅-C₂₀)alkenyl, wherein the atom marked with a * is connected to W¹ or —CH(Y⁴)—when W¹ is a covalent bond. In embodiments, X¹ is —(*C═O)—O-optionally substituted (C₁₀-C₂₀)alkenyl, wherein the atom marked with a * is connected to W¹ or —CH(Y⁴)—when W¹ is a covalent bond. In embodiments, X¹ is —(*C═O)—O-optionally substituted (C₁₀-C₁₃)alkenyl, wherein the atom marked with a * is connected to W¹ or —CH(Y⁴)—when W¹ is a covalent bond. In embodiments, X¹ is —*O—(C═O)-optionally substituted (C₃-C₂₅)alkyl, wherein the atom marked with a * is connected to W¹ or —CH(Y⁴)—when W¹ is a covalent bond. In embodiments, X¹ is —*O—(C═O)-optionally substituted (C₃-C₂₀)alkyl, wherein the atom marked with a * is connected to W¹ or —CH(Y⁴)—when W¹ is a covalent bond. In embodiments, X¹ is —*O—(C═O)-optionally substituted (C₅-C₂₀)alkyl, wherein the atom marked with a * is connected to W¹ or —CH(Y⁴)—when W¹ is a covalent bond. In embodiments, X¹ is —*O—(C═O)-optionally substituted (C₅-C₁₃)alkyl, wherein the atom marked with a * is connected to W¹ or —CH(Y⁴)—when W¹ is a covalent bond. In embodiments, X¹ is —*O—(C═O)-optionally substituted (C₃-C₂₅)alkenyl, wherein the atom marked with a * is connected to W¹ or —CH(Y⁴)—when W¹ is a covalent bond. In embodiments, X¹ is —*O—(C═O)-optionally substituted (C₃-C₂₀)alkenyl, wherein the atom marked with a * is connected to W¹ or —CH(Y⁴)—when W¹ is a covalent bond. In embodiments, X¹ is —*O—(C═O)-optionally substituted (C₅-C₂₀)alkenyl, wherein the atom marked with a * is connected to W¹ or —CH(Y⁴)—when W¹ is a covalent bond. In embodiments, X¹ is —*O—(C═O)-optionally substituted (C₁₀-C₂₀)alkenyl, wherein the atom marked with a * is connected to W¹ or —CH(Y⁴)—when W¹ is a covalent bond. In embodiments, X¹ is —*O—(C═O)-optionally substituted (C₁₀-C₁₃)alkenyl, wherein the atom marked with a * is connected to W¹ or —CH(Y⁴)—when W¹ is a covalent bond.

In embodiments, R^{13D} is optionally substituted (C₅-C₂₅)alkyl. In embodiments, R^{13D} is optionally substituted (C₅-C₂₀)alkyl. In embodiments, R^{13D} is optionally substituted (C₅-C₁₅)alkyl. In embodiments, R^{13D} is optionally substituted (C₆-C₁₂)alkyl. In embodiments, R^{13D} is optionally substituted (C₃-C₁₀)alkyl.

In embodiments, R^{13D} is optionally substituted (C₅-C₂₅)alkenyl. In embodiments, R^{13D} is optionally substituted (C₅-C₂₀)alkenyl. In embodiments, R^{13D} is optionally substituted (C₁₀-C₂₀)alkenyl. In embodiments, R^{13D} is optionally substituted (C₁₅-C₂₀)alkenyl.

In embodiments, R^{13D} is —W¹—X¹. In embodiments, W¹ is a covalent bond. In embodiments, W⁷ is optionally substituted (C₁-C₁₀)alkylene. In embodiments, W¹ is optionally substituted (C₁-C₃)alkylene. In embodiments, W¹ is optionally substituted (C₁-C₆)alkylene. In embodiments, W is optionally substituted (C₁-C₅)alkylene. In embodiments, W¹ is optionally substituted (C₂-C₁₀)alkenylene. In embodiments, W¹ is optionally substituted (C₂-C₃)alkenylene. In embodiments, W¹ is optionally substituted (C₂-C₆)alkenylene. In embodiments, W is optionally substituted (C₂-C₅)alkenylene. In embodiments, X¹ is —(*C═O)—O-optionally substituted (C₃-C₂₅)alkyl, wherein the atom marked with a * is connected to W¹ or —CH(Y⁴)—when W¹ is a covalent bond. In embodiments, X¹ is —(*C═O)—O-optionally substituted (C₃-C₂₀)alkyl, wherein the atom marked with a * is connected to W¹ or —CH(Y⁴)—when W¹ is a covalent bond. In embodiments, X¹ is —(*C═O)—O-optionally substituted (C₅-C₂₀)alkyl, wherein the atom marked with a * is connected to W¹ or —CH(Y⁴)—when W¹ is a covalent bond. In embodiments, X¹ is —(*C═O)—O-optionally substituted (C₅-C₁₃)alkyl, wherein the atom marked with a * is connected to W¹ or —CH(Y⁴)—when W¹ is a covalent bond. In embodiments, X¹ is —(*C═O)—O-optionally substituted (C₃-C₂₅)alkenyl, wherein the atom marked with a * is connected to W¹ or —CH(Y⁴)—when W¹ is a covalent bond. In embodiments, X¹ is —(*C═O)—O-optionally substituted (C₃-C₂₀)alkenyl, wherein the atom marked with a * is connected to W¹ or —CH(Y⁴)—when W¹ is a covalent bond. In embodiments, X¹ is —(*C═O)—O-optionally substituted (C₅-C₂₀)alkenyl, wherein the atom marked with a * is connected to W¹ or —CH(Y⁴)—when W¹ is a covalent bond. In embodiments, X¹ is —(*C═O)—O-optionally substituted (C₁₀-C₂₀)alkenyl, wherein the atom marked with a * is connected to W¹ or —CH(Y⁴)—when W¹ is a covalent bond. In embodiments, X¹ is —(*C═O)—O-optionally substituted (C₁₀-C₁₃)alkenyl, wherein the atom marked with a * is connected to W¹ or —CH(Y⁴)—when W¹ is a covalent bond. In embodiments, X¹ is —*O—(C═O)-optionally substituted (C₃-C₂₅)alkyl, wherein the atom marked with a * is connected to W¹ or —CH(Y⁴)—when W¹ is a covalent bond. In embodiments, X¹ is —*O—(C═O)-optionally substituted (C₃-C₂₀)alkyl, wherein the atom marked with a * is connected to W¹ or —CH(Y⁴)—when W¹ is a covalent bond. In embodiments, X¹ is —*O—(C═O)-optionally substituted (C₅-C₂₀)alkyl, wherein the atom marked with a * is connected to W¹ or —CH(Y⁴)—when W¹ is a covalent bond. In embodiments, X¹ is —*O—(C═O)-optionally substituted (C₅-C₁₃)alkyl, wherein the atom marked with a * is connected to W¹ or —CH(Y⁴)—when W¹ is a covalent bond. In embodiments, X¹ is —*O—(C═O)-optionally substituted (C₃-C₂₅)alkenyl, wherein the atom marked with a * is connected to W¹ or —CH(Y⁴)—when W¹ is a covalent bond. In embodiments, X¹ is —*O—(C═O)-optionally substituted (C₃-C₂₀)alkenyl, wherein the atom marked with a * is connected to W¹ or —CH(Y⁴)—when W¹ is a covalent bond. In embodiments, X¹ is —*O—(C═O)-optionally substituted (C₅-C₂₀)alkenyl, wherein the atom marked with a * is connected to W¹ or —CH(Y⁴)—when W¹ is a covalent bond. In embodiments, X¹ is —*O—(C═O)-optionally substituted (C₁₀-C₂₀)alkenyl, wherein the atom marked with a * is connected to W¹ or —CH(Y⁴)—when W¹ is a covalent bond. In embodiments, X¹ is —*O—(C═O)-optionally substituted (C₁₀-C₁₃)alkenyl, wherein the atom marked with a * is connected to W¹ or —CH(Y⁴)—when W¹ is a covalent bond.

In embodiments, each R^{13A}, R^{13B}, R^{13C} and R^{13D} is independently selected from:

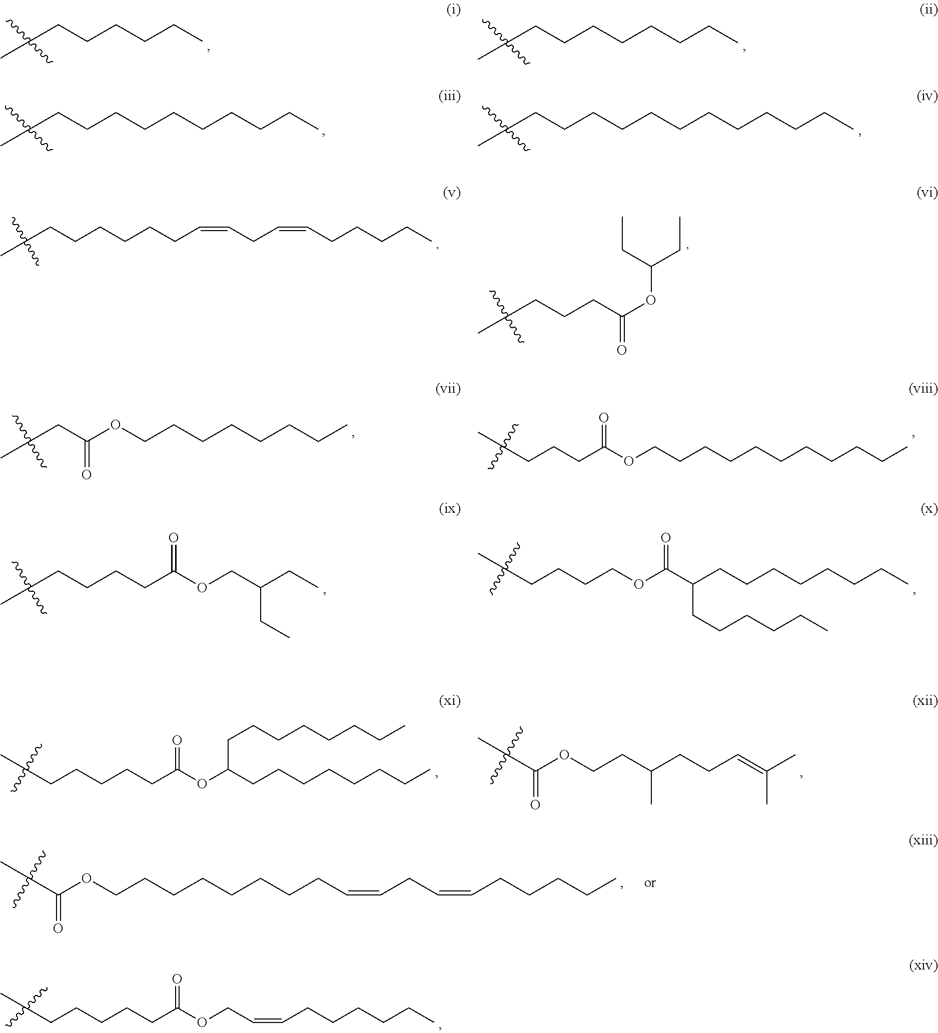

preferably wherein each R^{13A}, R^{13B}, R^{13C} and R^{13D} is option (ii).

In embodiments, each R^{13A}, R^{13B}, R^{13C} and R^{13D} is independently selected from:

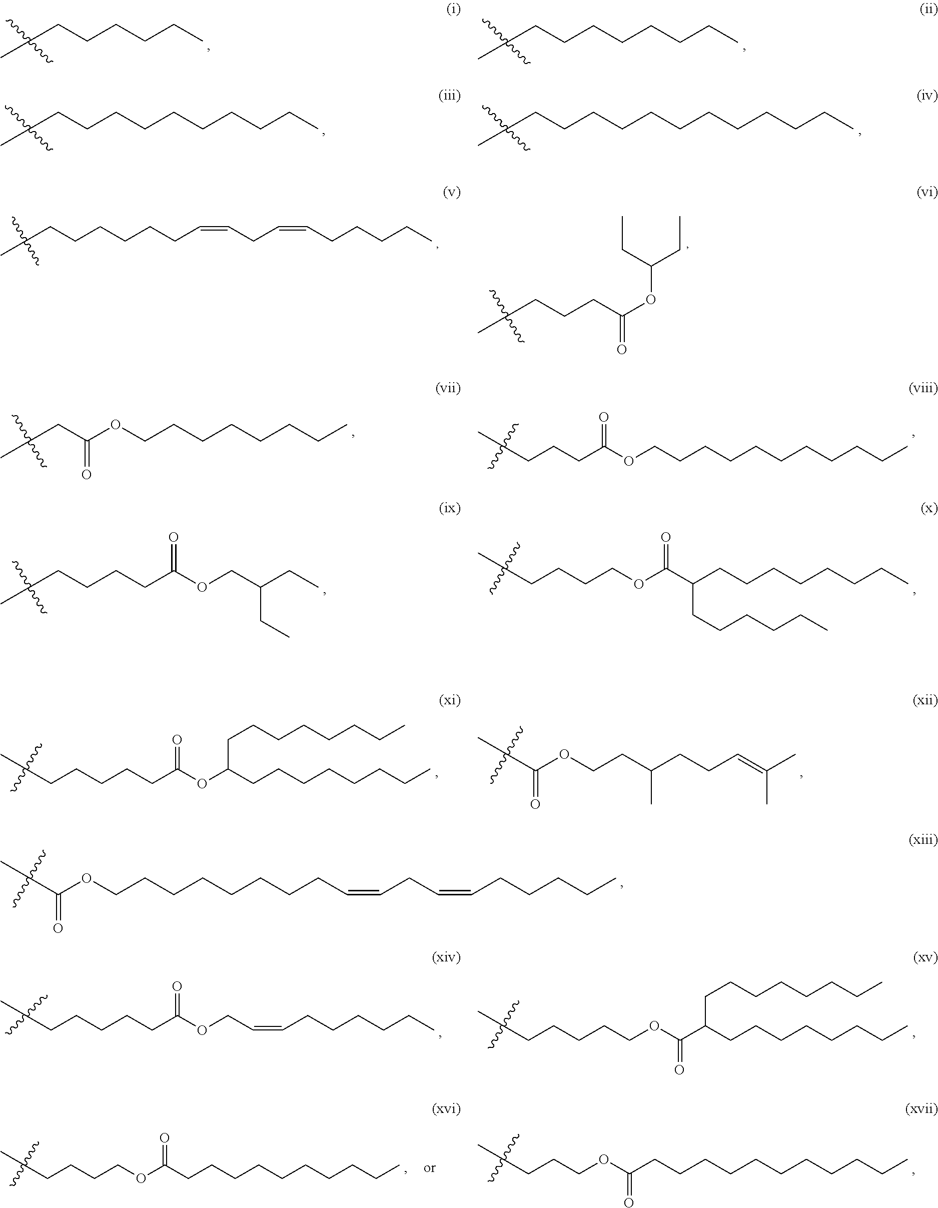

preferably wherein each R^{13A}, R^{13B}, R^{13C} and R^{13D} is option (ii), (iii), (viii), (X), (xiv) or (xv).

In embodiments, R^{13A}, R^{13B}, R^{13C} and R^{13D} are the same.

In embodiments, each W¹ in W¹—X¹ of any of the Formulae defined herein is independently selected from a covalent bond, optionally substituted (C₁-C₁₀)alkylene or optionally substituted (C₂-C₁₀)alkenylene; and
each X¹ in W¹—X¹ of any of the Formulae defined herein is independently selected from —(*C═O)—O-optionally substituted (C₃-C₂₅) branched alkyl, —(*C═O)—O-optionally substituted (C₃-C₂₅) branched alkenyl, —*O—(C═O)-optionally substituted (C₃-C₂₅) branched alkyl, or —*O—(C═O)-optionally substituted (C₃-C₂₅) branched alkenyl, wherein the atom marked with a * is connected to W¹ or —CH(Y¹)—when W¹ is a covalent bond.

In embodiments, the substituents are not optionally substituted.

In embodiments, the cationic lipids of the present invention have any one of the structures in Table A, or a pharmaceutically acceptable salt thereof.

In embodiments, the cationic lipids of the present invention have any one of the structures in Table B, or a pharmaceutically acceptable salt thereof.

In embodiments, provided herein is a composition comprising a cationic lipid of the present invention, and further comprising:
(i) one or more non-cationic lipids,
(ii) one or more cholesterol-based lipids and
(iii) one or more PEG-modified lipids.

In embodiments, this composition is a lipid nanoparticle, optionally a liposome. In embodiments, the one or more cationic lipid(s) constitute(s) about 30 mol %-60 mol % of the lipid nanoparticle. In embodiments, the one or more non-cationic lipid(s) constitute(s) 10 mol %-50 mol % of the lipid nanoparticle. In embodiments, the one or more PEG-modified lipid(s) constitute(s) 1 mol %-10 mol % of the lipid nanoparticle. In embodiments, the cholesterol-based lipid constitutes 10 mol %-50 mol % of the lipid nanoparticle.

In embodiments, the lipid nanoparticle encapsulates a nucleic acid, optionally an mRNA encoding a peptide or protein. In embodiments, the peptide is an antigen. In embodiments, the lipid nanoparticle encapsulates an mRNA encoding a peptide or protein. As used herein, the phrase “encapsulation percentage” refers to the fraction of therapeutic agent (e.g. mRNA) that is effectively encapsulated within a liposomal-based vehicle (e.g. a lipid nanoparticle) relative to the initial fraction of therapeutic agent present in the lipid phase. In embodiments, the lipid nanoparticles have an encapsulation percentage for mRNA of at least 50%. In embodiments, the lipid nanoparticles have an encapsulation percentage for mRNA of at least 55%. In embodiments, the lipid nanoparticles have an encapsulation percentage for mRNA of at least 60%. In embodiments, the lipid nanoparticles have an encapsulation percentage for mRNA of at least 65%. In embodiments, the lipid nanoparticles have an encapsulation percentage for mRNA of at least 70%. In embodiments, the lipid nanoparticles have an encapsulation percentage for mRNA of at least 75%. In embodiments, the lipid nanoparticles have an encapsulation percentage for mRNA of at least 80%. In embodiments, the lipid nanoparticles have an encapsulation percentage for mRNA of at least 85%. In embodiments, the lipid nanoparticles have an encapsulation percentage for mRNA of at least 90%. In embodiments, the lipid nanoparticles have an encapsulation percentage for mRNA of at least 95%. In embodiments, the encapsulation percentage is calculated by performing the Ribogreen assay (Invitrogen) with and without the presence of 0.1% Triton-X 100.

In embodiments, the composition of the present invention is for use in a vaccine.

In embodiments, the composition of the present invention is for use in therapy.

In embodiments, the composition of the present invention is for use in a method of treating or preventing a disease amenable to treatment or prevention by the peptide or protein encoded by the mRNA, optionally wherein the disease is (a) a protein deficiency, optionally wherein the protein deficiency affects the liver, lung, brain or muscle, (b) an autoimmune disease, (c) an infectious disease, or (d) cancer.

In embodiments, a method for treating or preventing a disease is provided, wherein said method comprises administering to a subject in need thereof a composition of the present invention and wherein the disease is amenable to treatment or prevention by the peptide or protein encoded by the mRNA, optionally wherein the disease is (a) a protein deficiency, optionally wherein the protein deficiency affects the liver, lung, brain or muscle, (b) an autoimmune disease, (c) an infectious disease, or (d) cancer.

In embodiments, the composition is administered intranasally, intravenously, intrathecally or intramuscularly, or by pulmonary delivery, optionally through nebulization. In embodiments, the composition is administered intramuscularly.

### Exemplary Compounds

In embodiments, the cationic lipids of the present invention include compounds selected from those depicted in Table A, or a pharmaceutically acceptable salt thereof.

Exemplary compounds include those described in Table A, or a pharmaceutically acceptable salt thereof.

TABLE A

| Compound | |
|---|---|
| Number | Structure of lipid |
| | Formula (IA) |

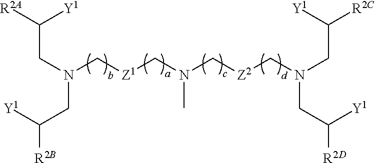

Formula (IA1)

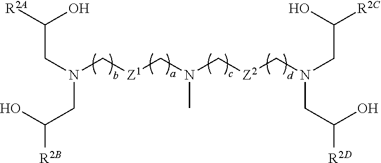

For example, wherein a is 1 or 2, b is 2, 3 or 4, c is 1 or 2 and d is 2, 3 or 4.

| I | 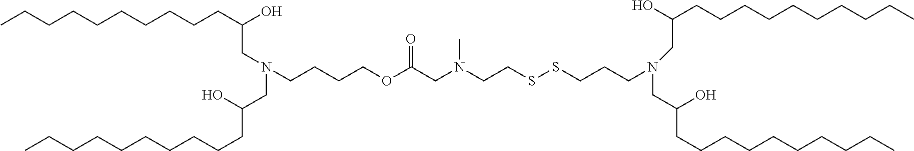 [SMILES: CCCCCCCCCCC(O)CN(CCCCOC(=O)CN(C)CCSSCCCN(CC(O)CCCCCCCCCC)CC(O)CCCCCCCCCC)CC(O)CCCCCCCCCC] |
|---|---|
| II | 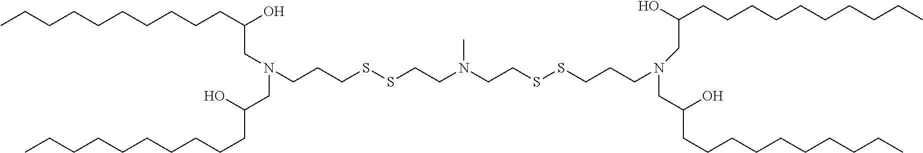 [SMILES: CCCCCCCCCCC(O)CN(CCCSSCCN(C)CCSSCCCN(CC(O)CCCCCCCCCC)CC(O)CCCCCCCCCC)CC(O)CCCCCCCCCC] |
| III | 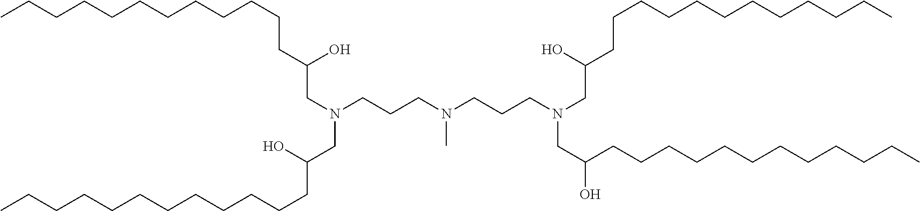 [SMILES: CCCCCCCCCCCCC(O)CN(CCCN(C)CCCN(CC(O)CCCCCCCCCCCC)CC(O)CCCCCCCCCCCC)CC(O)CCCCCCCCCCCC] |
| IV | 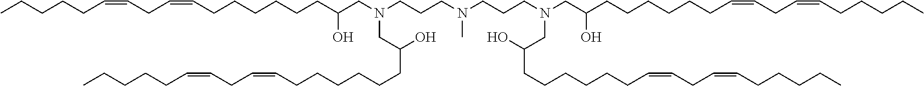 [SMILES: CCCCC/C=C\C/C=C\CCCCCCC(O)CN(CCCN(C)CCCN(CC(O)CCCCCC/C=C\C/C=C\CCCCC)CC(O)CCCCCC/C=C\C/C=C\CCCCC)CC(O)CCCCCC/C=C\C/C=C\CCCCC] |
| V | 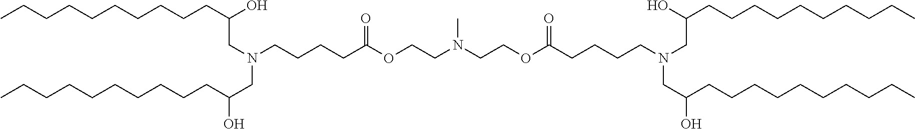 [SMILES: CCCCCCCCCCC(O)CN(CCCCC(=O)OCCN(C)CCOC(=O)CCCCN(CC(O)CCCCCCCCCC)CC(O)CCCCCCCCCC)CC(O)CCCCCCCCCC] |
| | Formula (IA1i) |

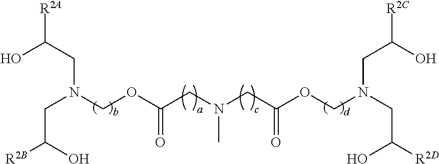

Formula (IA1ia)

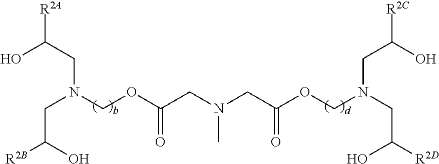

For example, wherein b is 3 or 4, and d is 3 or 4.

| VI | 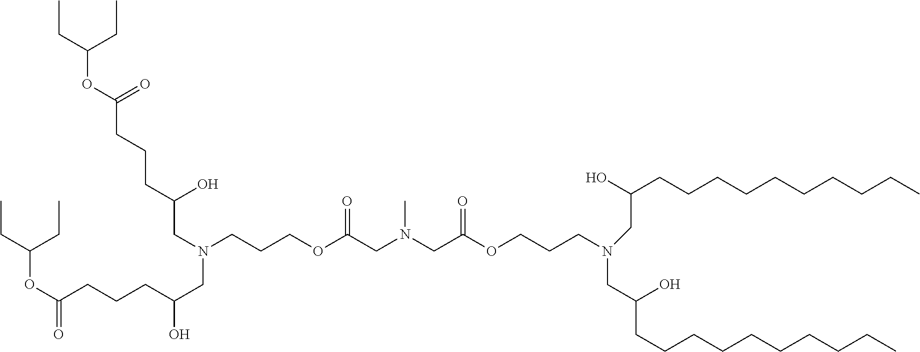 [SMILES: CCCCCCCCCCC(O)CN(CCCOC(=O)CN(C)CC(=O)OCCCN(CC(O)CCCC(=O)OC(CC)CC)CC(O)CCCC(=O)OC(CC)CC)CC(O)CCCCCCCCCC] |
|---|---|
| VII | 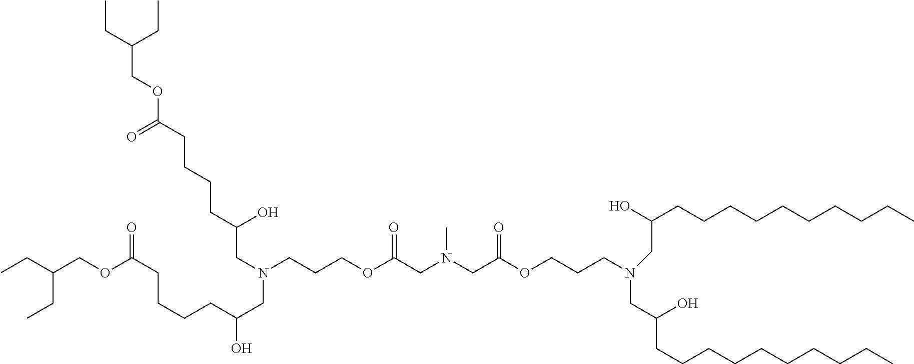 [SMILES: CCCCCCCCCCC(O)CN(CCCOC(=O)CN(C)CC(=O)OCCCN(CC(O)CCCCC(=O)OCC(CC)CC)CC(O)CCCCC(=O)OCC(CC)CC)CC(O)CCCCCCCCCC] |
| VIII | 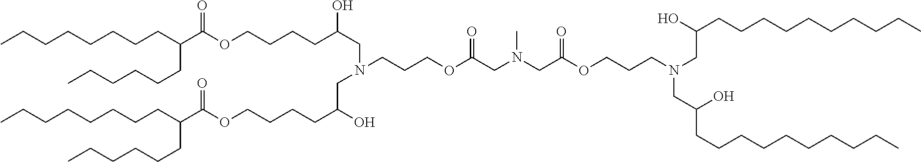 [SMILES: CCCCCCCCCCC(O)CN(CCCOC(=O)CN(C)CC(=O)OCCCN(CC(O)CCCCOC(=O)C(CCCCCC)CCCCCCCC)CC(O)CCCCOC(=O)C(CCCCCC)CCCCCCCC)CC(O)CCCCCCCCCC] |
| IX | 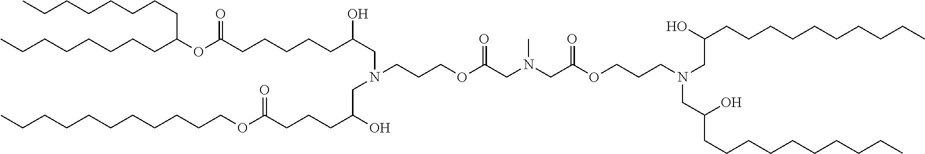 [SMILES: CCCCCCCCCCCOC(=O)CCCC(O)CN(CCCOC(=O)CN(C)CC(=O)OCCCN(CC(O)CCCCCCCCCC)CC(O)CCCCCCCCCC)CC(O)CCCCCC(=O)OC(CCCCCCCC)CCCCCCCC] |
| X | 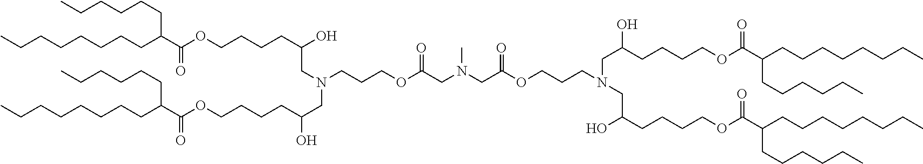 [SMILES: CCCCCCCCC(CCCCCC)C(=O)OCCCCC(O)CN(CCCOC(=O)CN(C)CC(=O)OCCCN(CC(O)CCCCOC(=O)C(CCCCCC)CCCCCCCC)CC(O)CCCCOC(=O)C(CCCCCC)CCCCCCCC)CC(O)CCCCOC(=O)C(CCCCCC)CCCCCCCC] |
| XI | 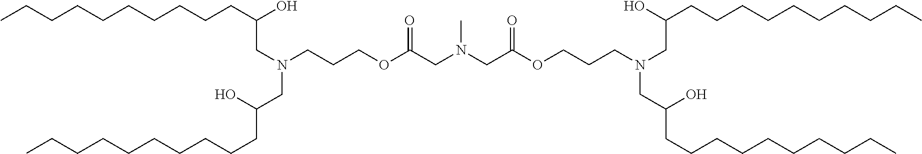 [SMILES: CCCCCCCCCCC(O)CN(CCCOC(=O)CN(C)CC(=O)OCCCN(CC(O)CCCCCCCCCC)CC(O)CCCCCCCCCC)CC(O)CCCCCCCCCC] |
| XII | 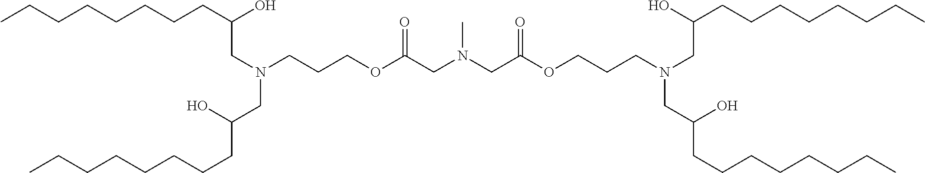 [SMILES: CCCCCCCCC(O)CN(CCCOC(=O)CN(C)CC(=O)OCCCN(CC(O)CCCCCCCC)CC(O)CCCCCCCC)CC(O)CCCCCCCC] |
| XIII | 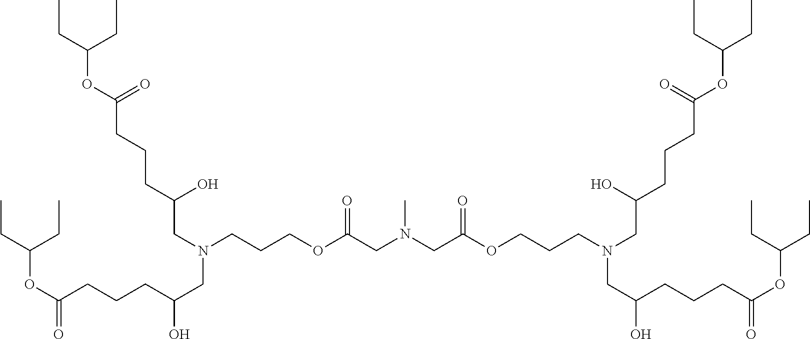 [SMILES: CCC(CC)OC(=O)CCCC(O)CN(CCCOC(=O)CN(C)CC(=O)OCCCN(CC(O)CCCC(=O)OC(CC)CC)CC(O)CCCC(=O)OC(CC)CC)CC(O)CCCC(=O)OC(CC)CC] |
| XIV | 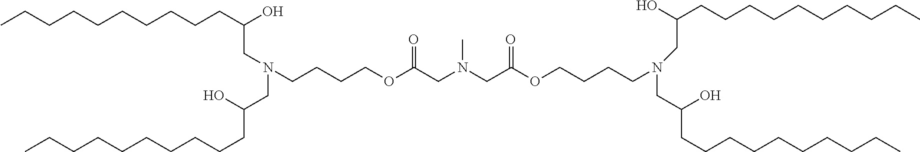 [SMILES: CCCCCCCCCCC(O)CN(CCCCOC(=O)CN(C)CC(=O)OCCCCN(CC(O)CCCCCCCCCC)CC(O)CCCCCCCCCC)CC(O)CCCCCCCCCC] |
| XV | 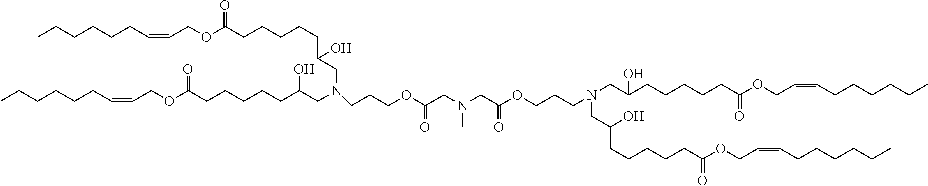 [SMILES: CCCCCC/C=C\COC(=O)CCCCCC(O)CN(CCCOC(=O)CN(C)CC(=O)OCCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC] |
| | Formula (IA2) |

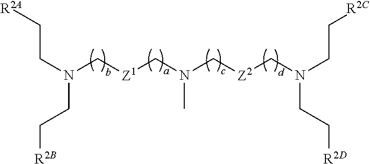

For example, wherein a is 1 or 2, b is 2, 3 or 4, c is 1 or 2 and d is 2, 3 or 4.

| XVI | 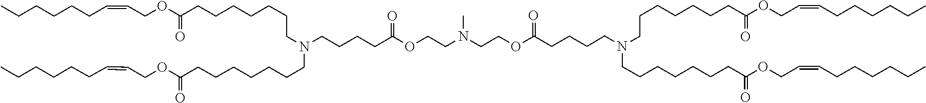 [SMILES: CCCCCC/C=C\COC(=O)CCCCCCCN(CCCCCCCC(=O)OC/C=C\CCCCCC)CCCCC(=O)OCCN(C)CCOC(=O)CCCCN(CCCCCCCC(=O)OC/C=C\CCCCCC)CCCCCCCC(=O)OC/C=C\CCCCCC] |
|---|---|
| XVII | 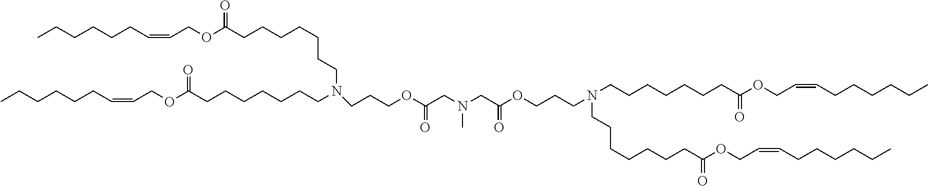 [SMILES: CCCCCC/C=C\COC(=O)CCCCCCCN(CCCCCCCC(=O)OC/C=C\CCCCCC)CCCOC(=O)CN(C)CC(=O)OCCCN(CCCCCCCC(=O)OC/C=C\CCCCCC)CCCCCCCC(=O)OC/C=C\CCCCCC] |
| XVIII | 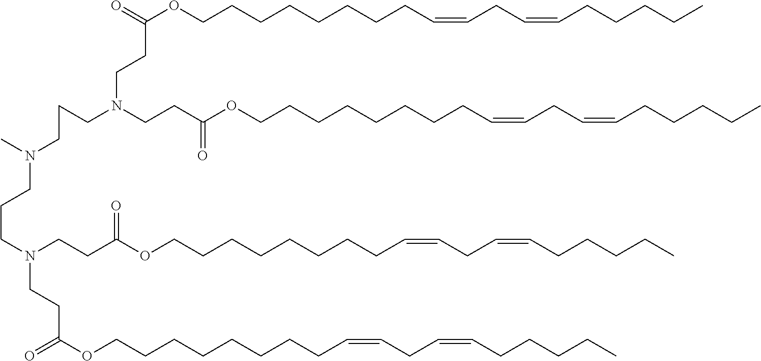 [SMILES: CCCCC/C=C\C/C=C\CCCCCCCCOC(=O)CCN(CCCN(C)CCCN(CCC(=O)OCCCCCCCC/C=C\C/C=C\CCCCC)CCC(=O)OCCCCCCCC/C=C\C/C=C\CCCCC)CCC(=O)OCCCCCCCC/C=C\C/C=C\CCCCC] |
| XIX | 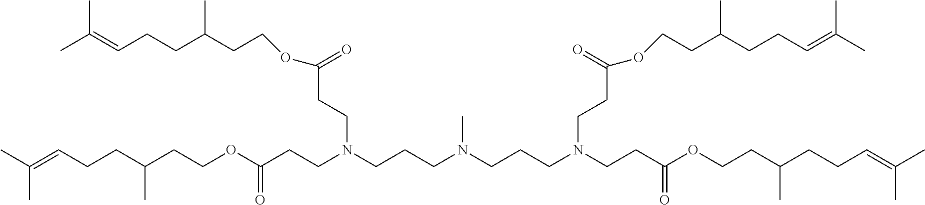 [SMILES: CC(C)=CCCC(C)CCOC(=O)CCN(CCCN(C)CCCN(CCC(=O)OCCC(C)CCC=C(C)C)CCC(=O)OCCC(C)CCC=C(C)C)CCC(=O)OCCC(C)CCC=C(C)C] |
| | Formula (IB) |

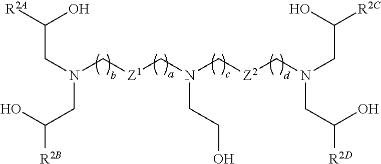

For example, wherein a is 1, b is 3, c is 1 and d is 3.

| XX | 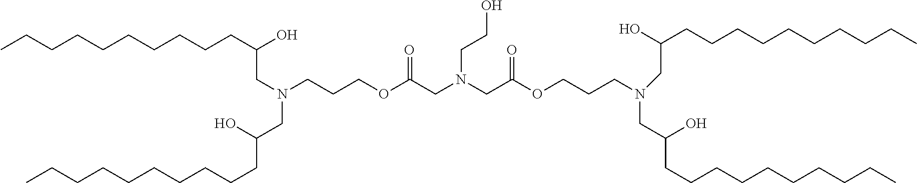 [SMILES: CCCCCCCCCCC(O)CN(CCCOC(=O)CN(CCO)CC(=O)OCCCN(CC(O)CCCCCCCCCC)CC(O)CCCCCCCCCC)CC(O)CCCCCCCCCC] |
|---|---|
| | Formula (IC) |

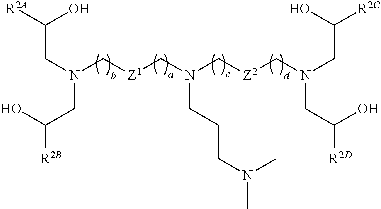

For example, wherein a is 2, b is 4, c is 2 and d is 4.

| XXI | 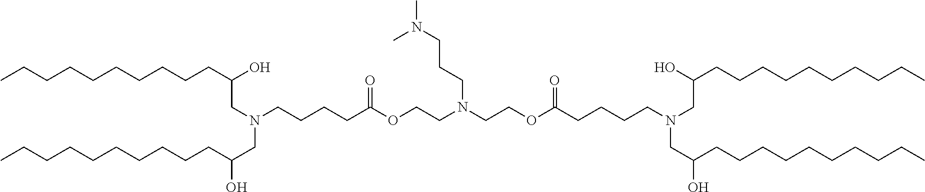 [SMILES: CCCCCCCCCCC(O)CN(CCCCC(=O)OCCN(CCCN(C)C)CCOC(=O)CCCCN(CC(O)CCCCCCCCCC)CC(O)CCCCCCCCCC)CC(O)CCCCCCCCCC] |
|---|---|
| | Formula (ID) |

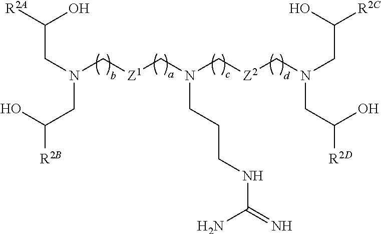

For example, wherein a is 2, b is 3, c is 2 and d is 3.

| XXII | 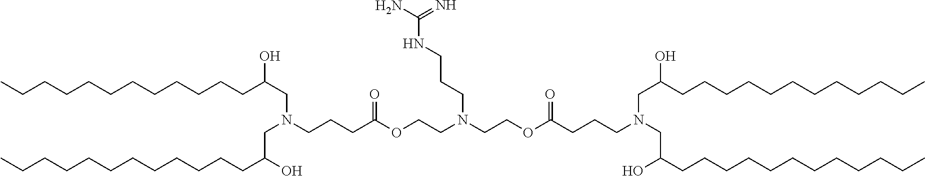 [SMILES: CCCCCCCCCCCCC(O)CN(CCCC(=O)OCCN(CCCNC(=N)N)CCOC(=O)CCCN(CC(O)CCCCCCCCCCCC)CC(O)CCCCCCCCCCCC)CC(O)CCCCCCCCCCCC] |
|---|---|
| XXIII | 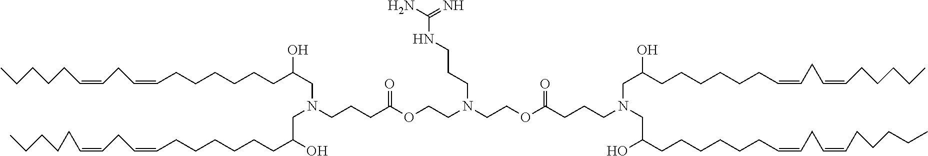 [SMILES: CCCCC/C=C\C/C=C\CCCCCCC(O)CN(CCCC(=O)OCCN(CCCNC(=N)N)CCOC(=O)CCCN(CC(O)CCCCCC/C=C\C/C=C\CCCCC)CC(O)CCCCCC/C=C\C/C=C\CCCCC)CC(O)CCCCCC/C=C\C/C=C\CCCCC] |
| | Formula (IE) |

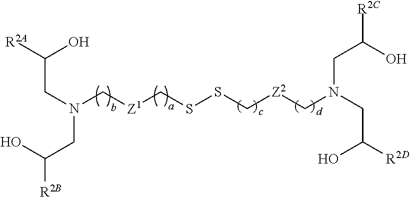

For example, wherein a is 2, b is 3, c is 2 and d is 3.

| XXIV | 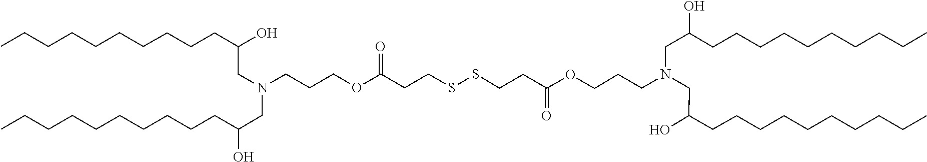 [SMILES: CCCCCCCCCCC(O)CN(CCCOC(=O)CCSSCCC(=O)OCCCN(CC(O)CCCCCCCCCC)CC(O)CCCCCCCCCC)CC(O)CCCCCCCCCC] |
|---|---|
| | Formula (IIA) |

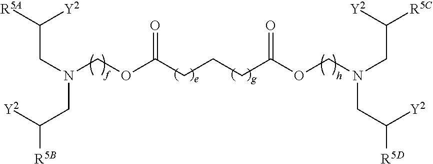

Formula (IIA1)

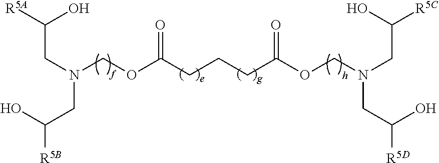

For example, wherein e is 0, 1 or 2, f is 3, g is 0 or 1 and h is 3

| XXVI | 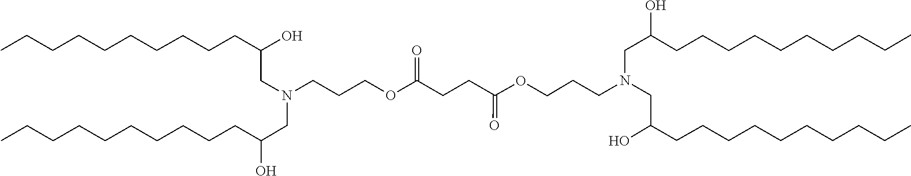 [SMILES: CCCCCCCCCCC(O)CN(CCCOC(=O)CCC(=O)OCCCN(CC(O)CCCCCCCCCC)CC(O)CCCCCCCCCC)CC(O)CCCCCCCCCC] |
|---|---|
| XXVII | 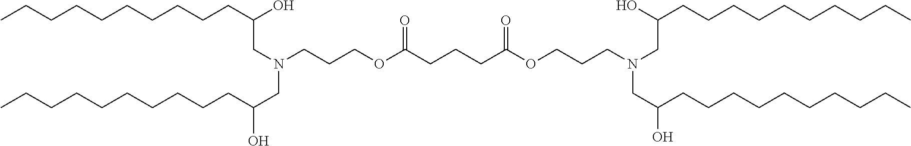 [SMILES: CCCCCCCCCCC(O)CN(CCCOC(=O)CCCC(=O)OCCCN(CC(O)CCCCCCCCCC)CC(O)CCCCCCCCCC)CC(O)CCCCCCCCCC] |
| XXVIII | 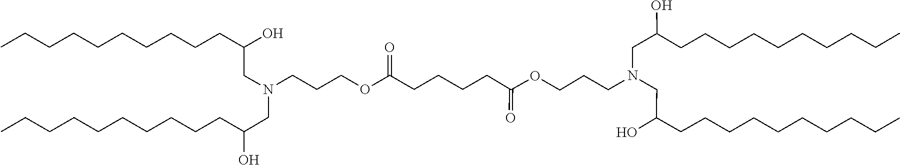 [SMILES: CCCCCCCCCCC(O)CN(CCCOC(=O)CCCCC(=O)OCCCN(CC(O)CCCCCCCCCC)CC(O)CCCCCCCCCC)CC(O)CCCCCCCCCC] |
| | Formula (IIA2) |

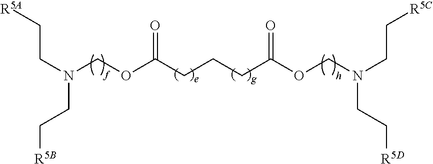

For example, wherein e is 1, f is 3, g is 0 and h is 3.

| XXIX | 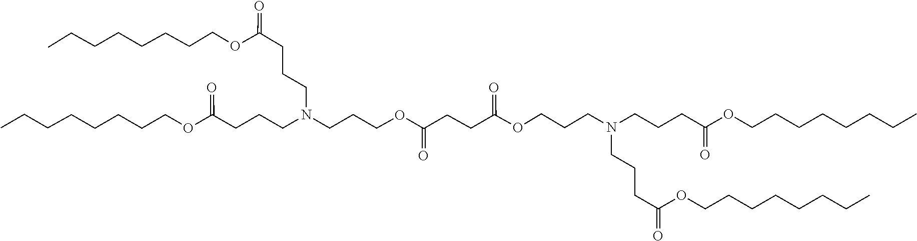 [SMILES: CCCCCCCCOC(=O)CCCN(CCCOC(=O)CCC(=O)OCCCN(CCCC(=O)OCCCCCCCC)CCCC(=O)OCCCCCCCC)CCCC(=O)OCCCCCCCC] |
|---|---|
| | Formula (IIB) |

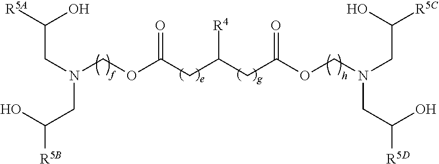

For example, wherein e is 0, 1 or 2, f is 3, g is 0 or 1 and h is 3

| XXX | 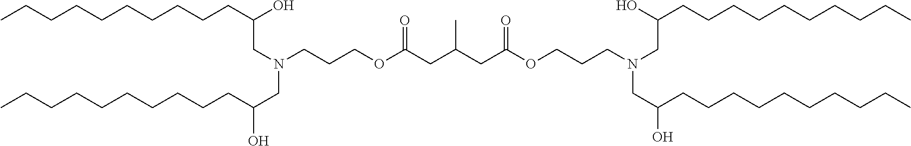 [SMILES: CCCCCCCCCCC(O)CN(CCCOC(=O)CC(C)CC(=O)OCCCN(CC(O)CCCCCCCCCC)CC(O)CCCCCCCCCC)CC(O)CCCCCCCCCC] |
|---|---|
| XXXI | 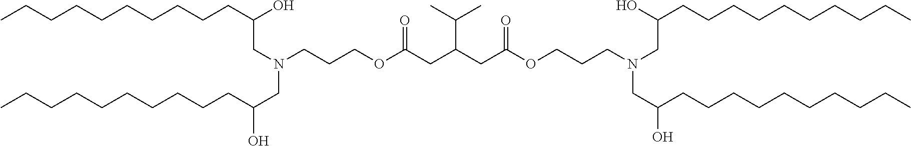 [SMILES: CCCCCCCCCCC(O)CN(CCCOC(=O)CC(CC(=O)OCCCN(CC(O)CCCCCCCCCC)CC(O)CCCCCCCCCC)C(C)C)CC(O)CCCCCCCCCC] |
| XXXII | 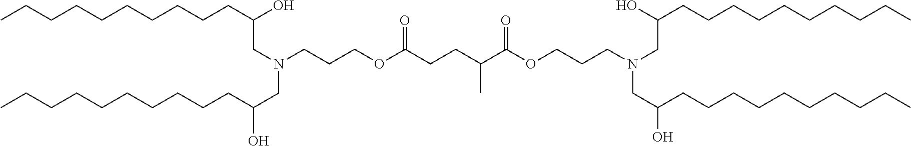 [SMILES: CCCCCCCCCCC(O)CN(CCCOC(=O)CCC(C)C(=O)OCCCN(CC(O)CCCCCCCCCC)CC(O)CCCCCCCCCC)CC(O)CCCCCCCCCC] |
| XXXIII | 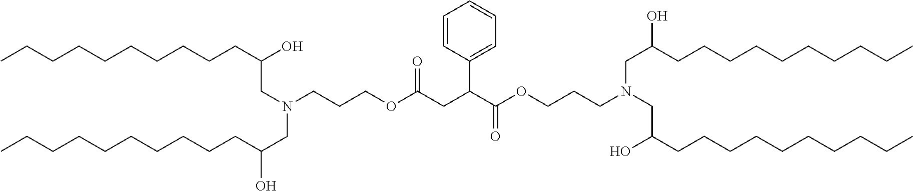 [SMILES: CCCCCCCCCCC(O)CN(CCCOC(=O)CC(C(=O)OCCCN(CC(O)CCCCCCCCCC)CC(O)CCCCCCCCCC)c1ccccc1)CC(O)CCCCCCCCCC] |
| XXXIV | 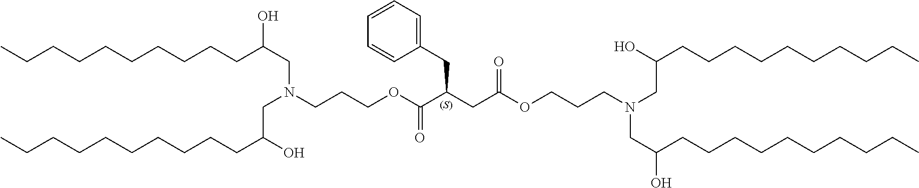 |
| | Formula (IIC) |

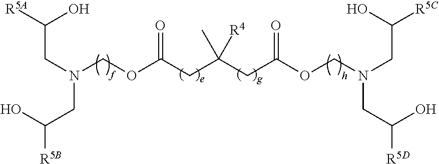

For example, wherein e is 1, f is 3, g is 1 and h is 3.

| XXXV | 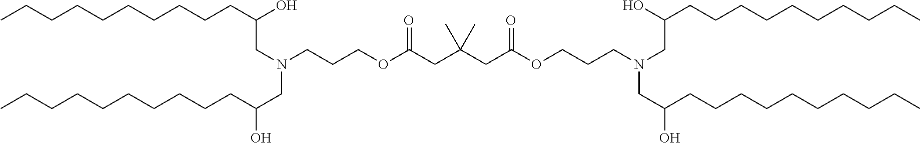 [SMILES: CCCCCCCCCCC(O)CN(CCCOC(=O)CC(C)(C)CC(=O)OCCCN(CC(O)CCCCCCCCCC)CC(O)CCCCCCCCCC)CC(O)CCCCCCCCCC] |
|---|---|
| XXXVI | 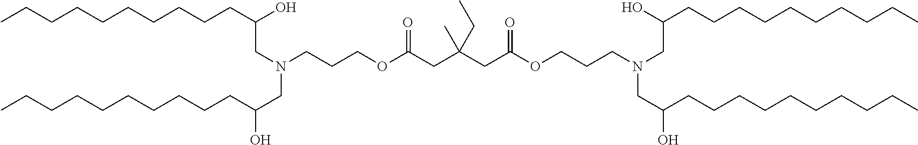 [SMILES: CCCCCCCCCCC(O)CN(CCCOC(=O)CC(C)(CC)CC(=O)OCCCN(CC(O)CCCCCCCCCC)CC(O)CCCCCCCCCC)CC(O)CCCCCCCCCC] |
| | Formula (IIIC1) |

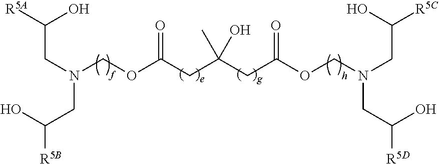

Formula (IIC1i)

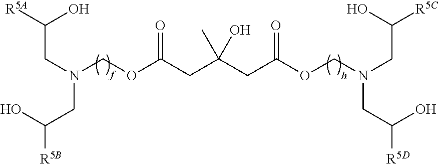

For example, wherein f is 3, 4, 5 or 6 and h is 3, 4, 5 or 6.

| XXXVII | 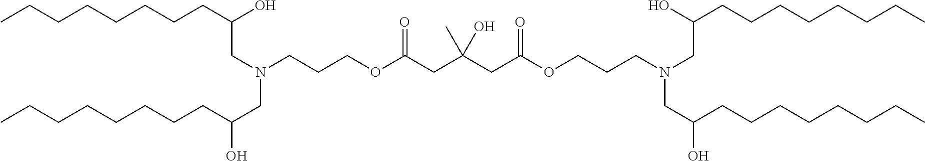 [SMILES: CCCCCCCCC(O)CN(CCCOC(=O)CC(C)(O)CC(=O)OCCCN(CC(O)CCCCCCCC)CC(O)CCCCCCCC)CC(O)CCCCCCCC] |
|---|---|
| XXXVIII | 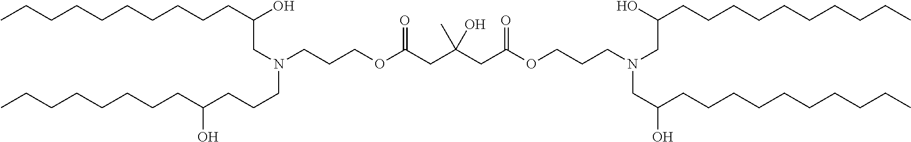 [SMILES: CCCCCCCCCCC(O)CN(CCCOC(=O)CC(C)(O)CC(=O)OCCCN(CC(O)CCCCCCCCCC)CC(O)CCCCCCCCCC)CCCC(O)CCCCCCCC] |
| XXXIX | 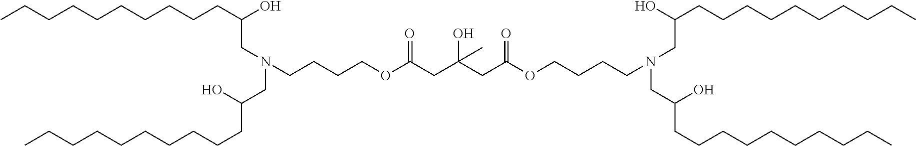 [SMILES: CCCCCCCCCCC(O)CN(CCCCOC(=O)CC(C)(O)CC(=O)OCCCCN(CC(O)CCCCCCCCCC)CC(O)CCCCCCCCCC)CC(O)CCCCCCCCCC] |
| XL | 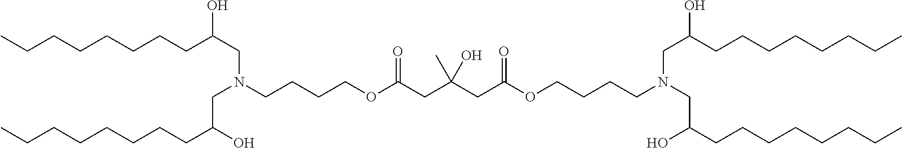 [SMILES: CCCCCCCCC(O)CN(CCCCOC(=O)CC(C)(O)CC(=O)OCCCCN(CC(O)CCCCCCCC)CC(O)CCCCCCCC)CC(O)CCCCCCCC] |
| XLI | 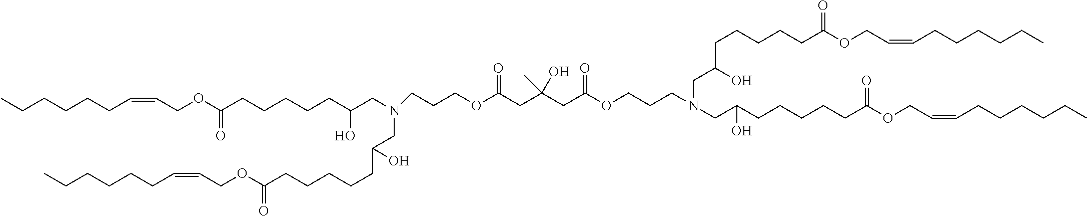 [SMILES: CCCCCC/C=C\COC(=O)CCCCCC(O)CN(CCCOC(=O)CC(C)(O)CC(=O)OCCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC] |
| LXXII | 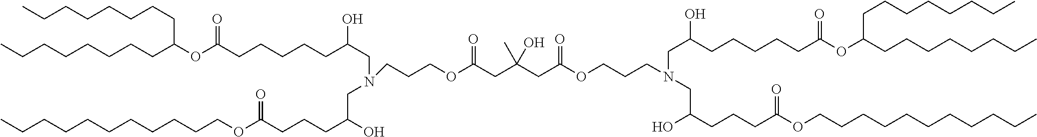 [SMILES: CCCCCCCCCCCOC(=O)CCCC(O)CN(CCCOC(=O)CC(C)(O)CC(=O)OCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| XLII | 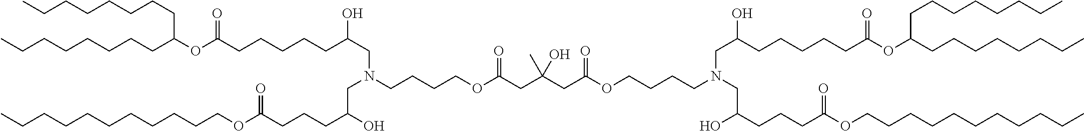 [SMILES: CCCCCCCCCCCOC(=O)CCCC(O)CN(CCCCOC(=O)CC(C)(O)CC(=O)OCCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| XLIII | 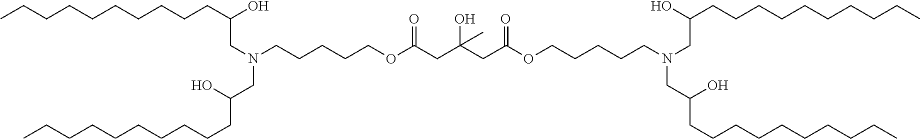 [SMILES: CCCCCCCCCCC(O)CN(CCCCCOC(=O)CC(C)(O)CC(=O)OCCCCCN(CC(O)CCCCCCCCCC)CC(O)CCCCCCCCCC)CC(O)CCCCCCCCCC] |
| XLIV | 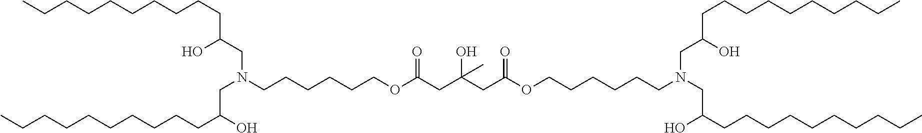 [SMILES: CCCCCCCCCCC(O)CN(CCCCCCOC(=O)CC(C)(O)CC(=O)OCCCCCCN(CC(O)CCCCCCCCCC)CC(O)CCCCCCCCCC)CC(O)CCCCCCCCCC] |
| XLV | 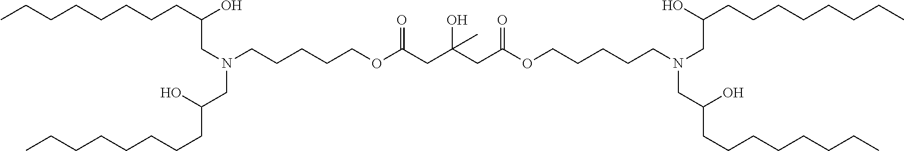 [SMILES: CCCCCCCCC(O)CN(CCCCCOC(=O)CC(C)(O)CC(=O)OCCCCCN(CC(O)CCCCCCCC)CC(O)CCCCCCCC)CC(O)CCCCCCCC] |
| XLVI | 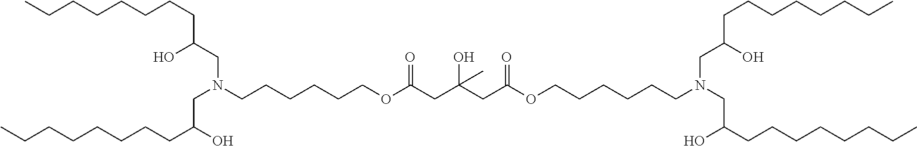 [SMILES: CCCCCCCCC(O)CN(CCCCCCOC(=O)CC(C)(O)CC(=O)OCCCCCCN(CC(O)CCCCCCCC)CC(O)CCCCCCCC)CC(O)CCCCCCCC] |
| XLVII | 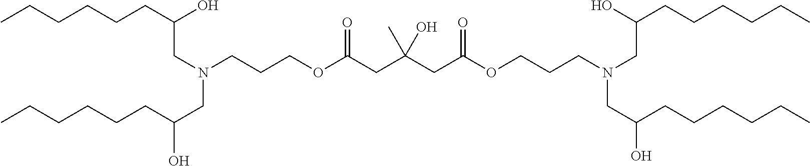 [SMILES: CCCCCCC(O)CN(CCCOC(=O)CC(C)(O)CC(=O)OCCCN(CC(O)CCCCCC)CC(O)CCCCCC)CC(O)CCCCCC] |
| XLVIII | 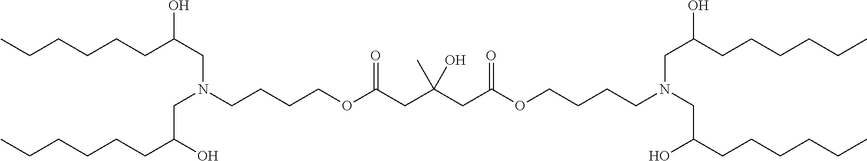 [SMILES: CCCCCCC(O)CN(CCCCOC(=O)CC(C)(O)CC(=O)OCCCCN(CC(O)CCCCCC)CC(O)CCCCCC)CC(O)CCCCCC] |
| XLIX | 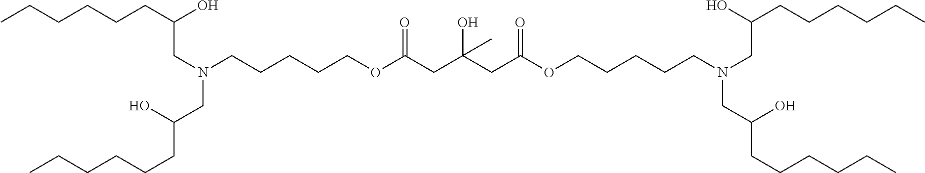 [SMILES: CCCCCCC(O)CN(CCCCCOC(=O)CC(C)(O)CC(=O)OCCCCCN(CC(O)CCCCCC)CC(O)CCCCCC)CC(O)CCCCCC] |
| L | 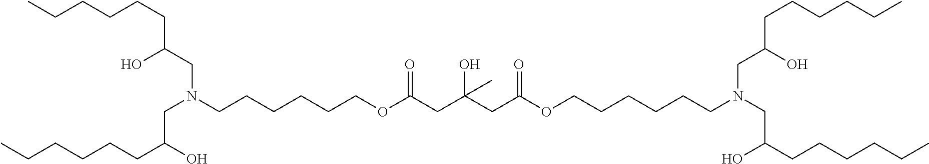 [SMILES: CCCCCCC(O)CN(CCCCCCOC(=O)CC(C)(O)CC(=O)OCCCCCCN(CC(O)CCCCCC)CC(O)CCCCCC)CC(O)CCCCCC] |
| LI | 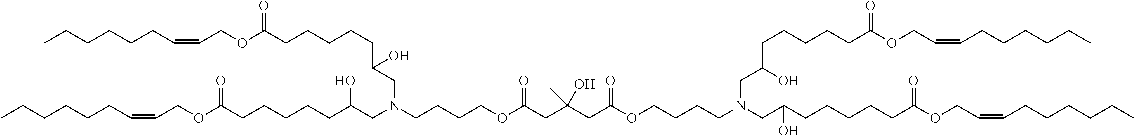 [SMILES: CCCCCC/C=C\COC(=O)CCCCCC(O)CN(CCCCOC(=O)CC(C)(O)CC(=O)OCCCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC] |
| | Formula (III) |

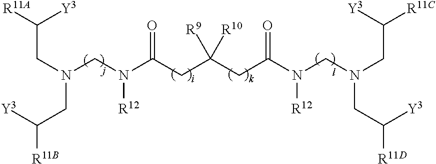

Formula (IIIA)

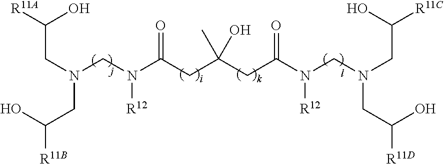

Formula (IIIA1)

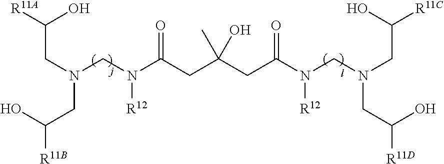

For example, wherein j is 3 or 4 and I is 3 or 4.

| LII | 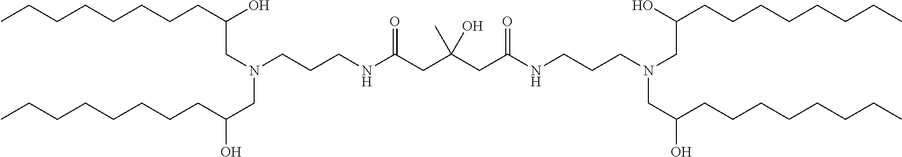 [SMILES: CCCCCCCCC(O)CN(CCCNC(=O)CC(C)(O)CC(=O)NCCCN(CC(O)CCCCCCCC)CC(O)CCCCCCCC)CC(O)CCCCCCCC] |
|---|---|
| LIII | 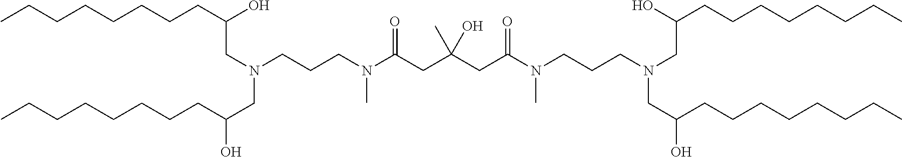 [SMILES: CCCCCCCCC(O)CN(CCCN(C)C(=O)CC(C)(O)CC(=O)N(C)CCCN(CC(O)CCCCCCCC)CC(O)CCCCCCCC)CC(O)CCCCCCCC] |
| LIV | 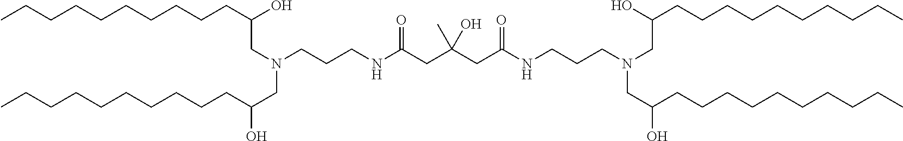 [SMILES: CCCCCCCCCCC(O)CN(CCCNC(=O)CC(C)(O)CC(=O)NCCCN(CC(O)CCCCCCCCCC)CC(O)CCCCCCCCCC)CC(O)CCCCCCCCCC] |
| LV | 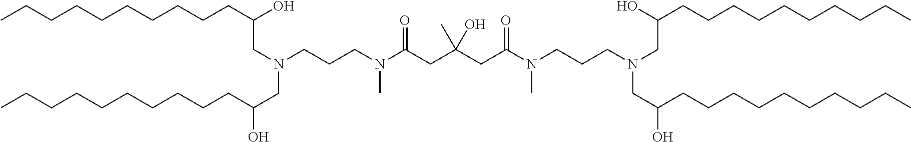 [SMILES: CCCCCCCCCCC(O)CN(CCCN(C)C(=O)CC(C)(O)CC(=O)N(C)CCCN(CC(O)CCCCCCCCCC)CC(O)CCCCCCCCCC)CC(O)CCCCCCCCCC] |
| LVI | 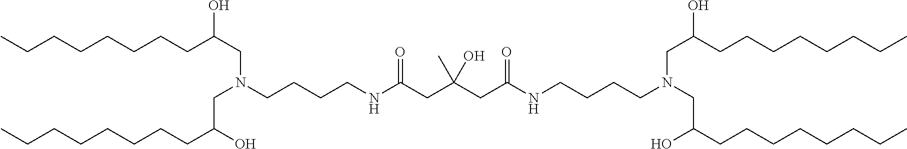 [SMILES: CCCCCCCCC(O)CN(CCCCNC(=O)CC(C)(O)CC(=O)NCCCCN(CC(O)CCCCCCCC)CC(O)CCCCCCCC)CC(O)CCCCCCCC] |
| LVII | 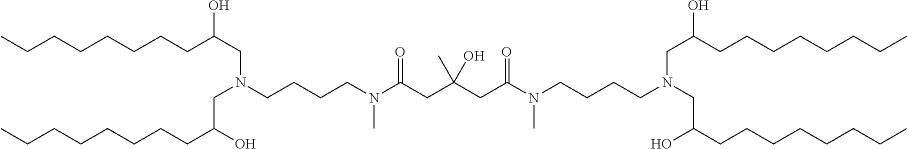 [SMILES: CCCCCCCCC(O)CN(CCCCN(C)C(=O)CC(C)(O)CC(=O)N(C)CCCCN(CC(O)CCCCCCCC)CC(O)CCCCCCCC)CC(O)CCCCCCCC] |
| LVIII | 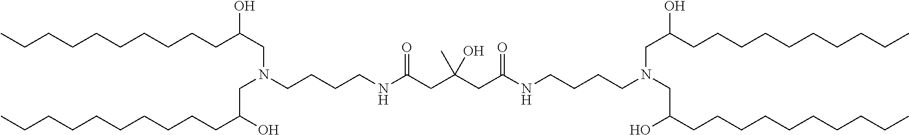 [SMILES: CCCCCCCCCCC(O)CN(CCCCNC(=O)CC(C)(O)CC(=O)NCCCCN(CC(O)CCCCCCCCCC)CC(O)CCCCCCCCCC)CC(O)CCCCCCCCCC] |
| LIX | 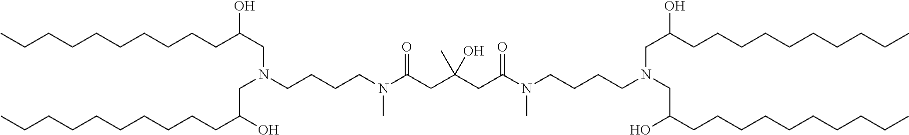 [SMILES: CCCCCCCCCCC(O)CN(CCCCN(C)C(=O)CC(C)(O)CC(=O)N(C)CCCCN(CC(O)CCCCCCCCCC)CC(O)CCCCCCCCCC)CC(O)CCCCCCCCCC] |
| | Formula (IV) |

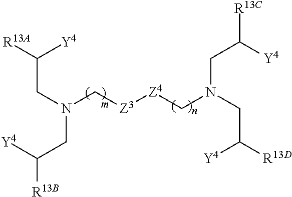

Formula (IVA)

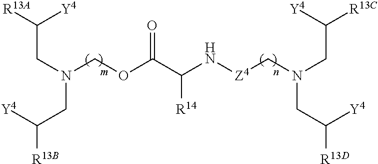

Formula (IVA1)

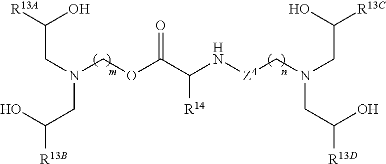

For example, wherein m is 4 and n is 4 or 5.

| LX | 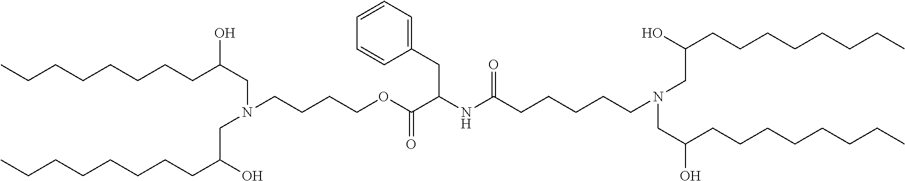 [SMILES: CCCCCCCCC(O)CN(CCCCCC(=O)NC(Cc1ccccc1)C(=O)OCCCCN(CC(O)CCCCCCCC)CC(O)CCCCCCCC)CC(O)CCCCCCCC] |
|---|---|
| LXI | 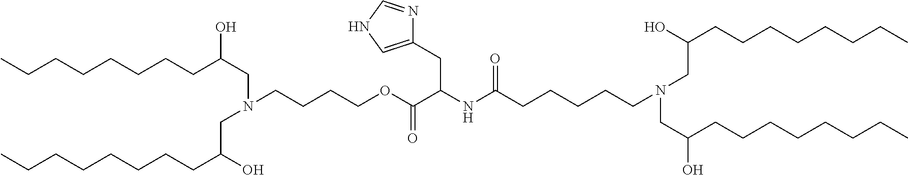 [SMILES: CCCCCCCCC(O)CN(CCCCCC(=O)NC(Cc1c[nH]cn1)C(=O)OCCCCN(CC(O)CCCCCCCC)CC(O)CCCCCCCC)CC(O)CCCCCCCC] |
| LXII | 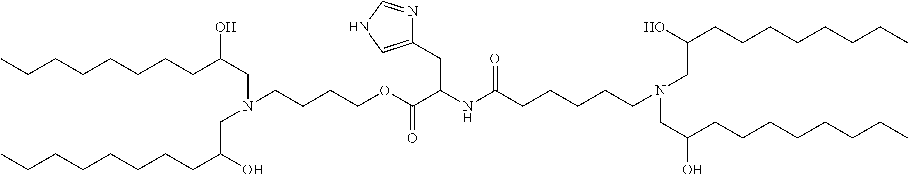 [SMILES: CCCCCCCCC(O)CN(CCCCCC(=O)NC(Cc1c[nH]cn1)C(=O)OCCCCN(CC(O)CCCCCCCC)CC(O)CCCCCCCC)CC(O)CCCCCCCC] |
| LXIII | 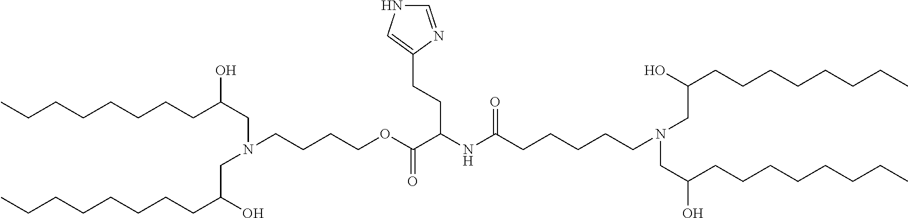 [SMILES: CCCCCCCCC(O)CN(CCCCCC(=O)NC(CCc1c[nH]cn1)C(=O)OCCCCN(CC(O)CCCCCCCC)CC(O)CCCCCCCC)CC(O)CCCCCCCC] |
| LXIV | 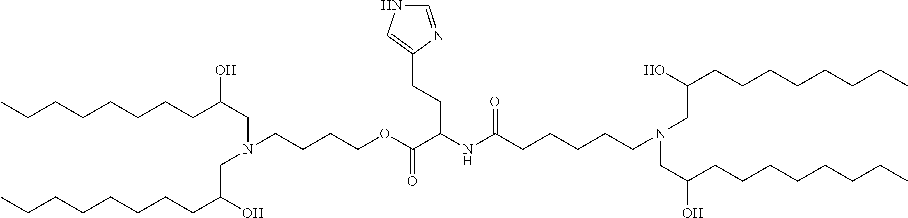 [SMILES: CCCCCCCCC(O)CN(CCCCCC(=O)NC(CCc1c[nH]cn1)C(=O)OCCCCN(CC(O)CCCCCCCC)CC(O)CCCCCCCC)CC(O)CCCCCCCC] |
| LXXIII | 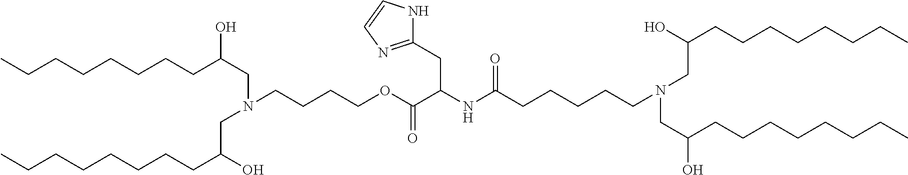 [SMILES: CCCCCCCCC(O)CN(CCCCCC(=O)NC(Cc1ncc[nH]1)C(=O)OCCCCN(CC(O)CCCCCCCC)CC(O)CCCCCCCC)CC(O)CCCCCCCC] |
| LXXIV | 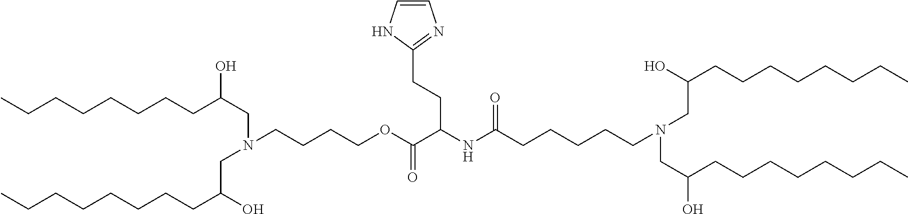 [SMILES: CCCCCCCCC(O)CN(CCCCCC(=O)NC(CCc1ncc[nH]1)C(=O)OCCCCN(CC(O)CCCCCCCC)CC(O)CCCCCCCC)CC(O)CCCCCCCC] |
| | Formula (IIA1) |

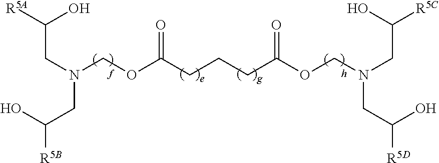

Formula (IIA1i)

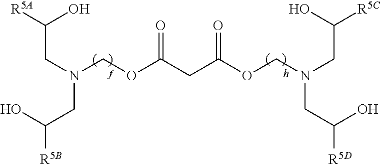

For example, wherein f is 3, 4 or 5 and h is 3, 4 or 5

| XXV | 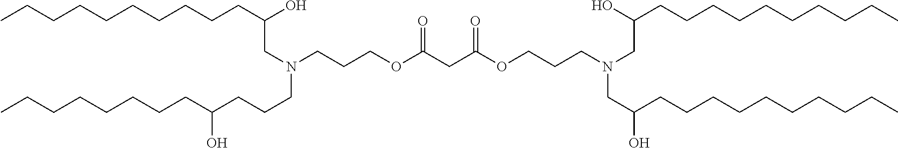 [SMILES: CCCCCCCCCCC(O)CN(CCCOC(=O)CC(=O)OCCCN(CC(O)CCCCCCCCCC)CC(O)CCCCCCCCCC)CCCC(O)CCCCCCCC] |
|---|---|
| LXV | 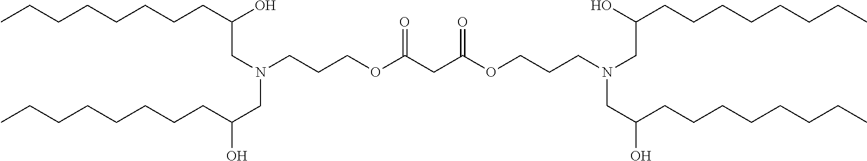 [SMILES: CCCCCCCCC(O)CN(CCCOC(=O)CC(=O)OCCCN(CC(O)CCCCCCCC)CC(O)CCCCCCCC)CC(O)CCCCCCCC] |
| LXVI | 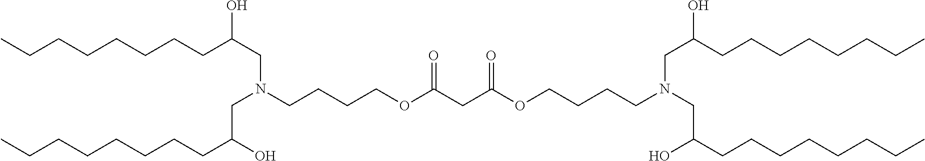 [SMILES: CCCCCCCCC(O)CN(CCCCOC(=O)CC(=O)OCCCCN(CC(O)CCCCCCCC)CC(O)CCCCCCCC)CC(O)CCCCCCCC] |
| LXVII | 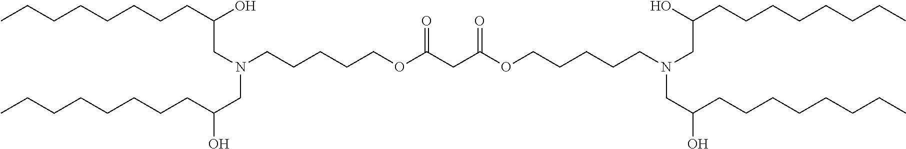 [SMILES: CCCCCCCCC(O)CN(CCCCCOC(=O)CC(=O)OCCCCCN(CC(O)CCCCCCCC)CC(O)CCCCCCCC)CC(O)CCCCCCCC] |
| | Formula (IID) |

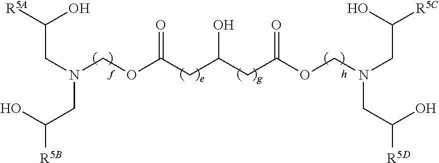

Formula (IID1)

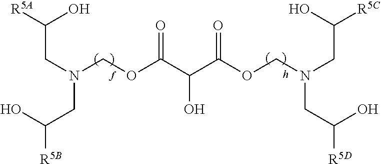

For example, wherein f is 3 or 4 and h is 3 or 4

| LXVIII | 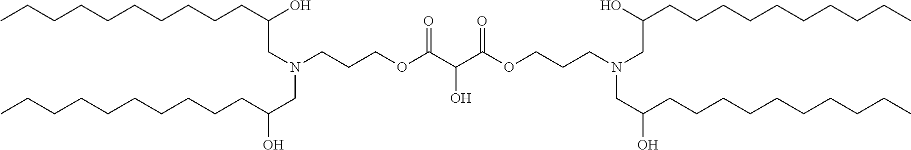 [SMILES: CCCCCCCCCCC(O)CN(CCCOC(=O)C(O)C(=O)OCCCN(CC(O)CCCCCCCCCC)CC(O)CCCCCCCCCC)CC(O)CCCCCCCCCC] |
|---|---|
| LXIX | 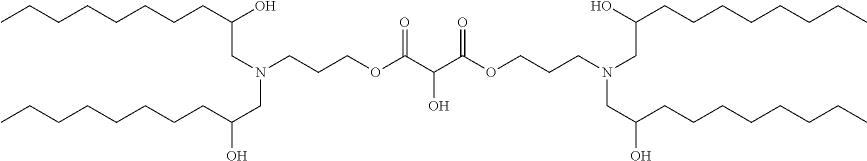 [SMILES: CCCCCCCCC(O)CN(CCCOC(=O)C(O)C(=O)OCCCN(CC(O)CCCCCCCC)CC(O)CCCCCCCC)CC(O)CCCCCCCC] |
| LXX | 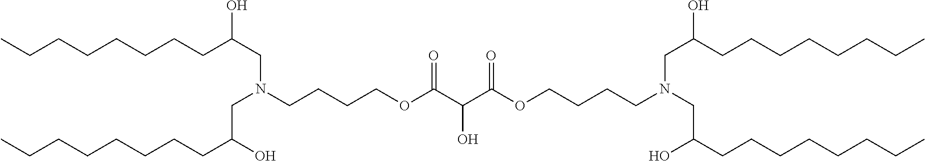 [SMILES: CCCCCCCCC(O)CN(CCCCOC(=O)C(O)C(=O)OCCCCN(CC(O)CCCCCCCC)CC(O)CCCCCCCC)CC(O)CCCCCCCC] |
| | Formula (IIE) |

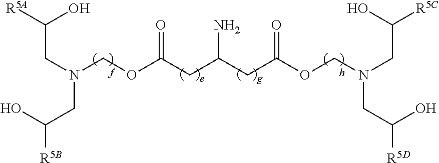

Formula (IIE1)

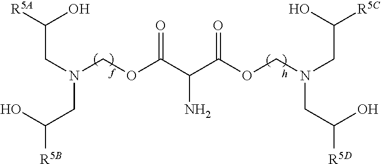

For example, wherein f is 3 and h is 3

| | |
|---|---|
| LXXI | 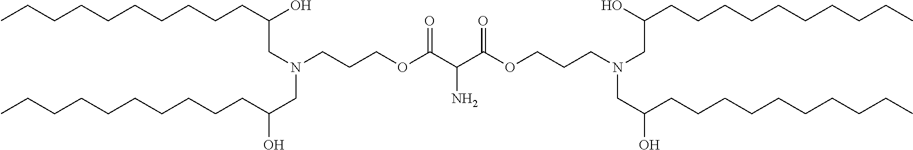 [SMILES: CCCCCCCCCCC(O)CN(CCCOC(=O)C(N)C(=O)OCCCN(CC(O)CCCCCCCCCC)CC(O)CCCCCCCCCC)CC(O)CCCCCCCCCC] |

Any of the compounds identified in Table A above may be provided in the form of a pharmaceutically acceptable salt and such salts are intended to be encompassed by the present invention.

In embodiments, the cationic lipids of the present invention include compounds selected from those depicted in Table B, or a pharmaceutically acceptable salt thereof.

Exemplary compounds include those described in Table B, or a pharmaceutically acceptable salt thereof.

TABLE B

| | |
|---|---|
| Compound | |
| Number | Structure of lipid |

Formula (IA1ii): 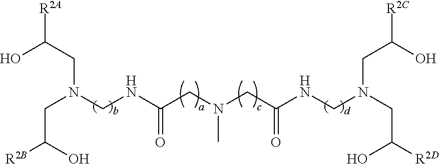 For example, wherein b is 3 or 4 and d is 3 or 4
Formula (IA1iia): 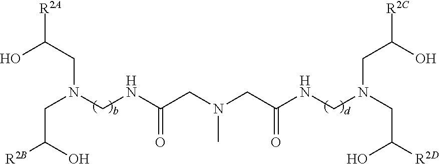 For example, wherein b is 3 or 4 and d is 3 or 4

| | |
|---|---|
| LXXV | 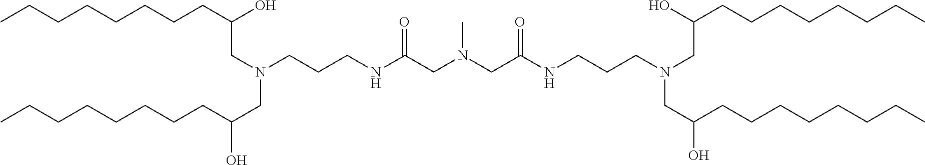 [SMILES: CCCCCCCCC(O)CN(CCCNC(=O)CN(C)CC(=O)NCCCN(CC(O)CCCCCCCC)CC(O)CCCCCCCC)CC(O)CCCCCCCC] |
| LXXVI | 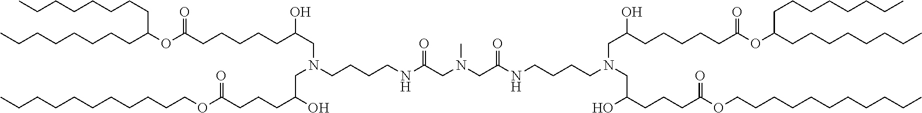 [SMILES: CCCCCCCCCCCOC(=O)CCCC(O)CN(CCCCNC(=O)CN(C)CC(=O)NCCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| LXXVIII | 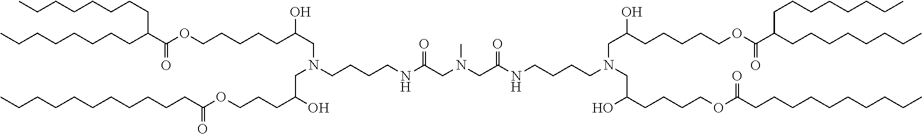 [SMILES: CCCCCCCCCCCC(=O)OCCCC(O)CN(CCCCNC(=O)CN(C)CC(=O)NCCCCN(CC(O)CCCCCOC(=O)C(CCCCCCCC)CCCCCCCC)CC(O)CCCCOC(=O)CCCCCCCCCC)CC(O)CCCCCOC(=O)C(CCCCCCCC)CCCCCCCC] |
| LXXIX | 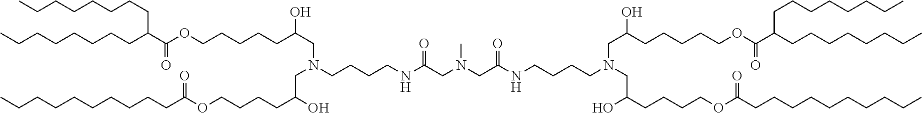 [SMILES: CCCCCCCCCCC(=O)OCCCCC(O)CN(CCCCNC(=O)CN(C)CC(=O)NCCCCN(CC(O)CCCCCOC(=O)C(CCCCCCCC)CCCCCCCC)CC(O)CCCCOC(=O)CCCCCCCCCC)CC(O)CCCCCOC(=O)C(CCCCCCCC)CCCCCCCC] |
| LXXXI | 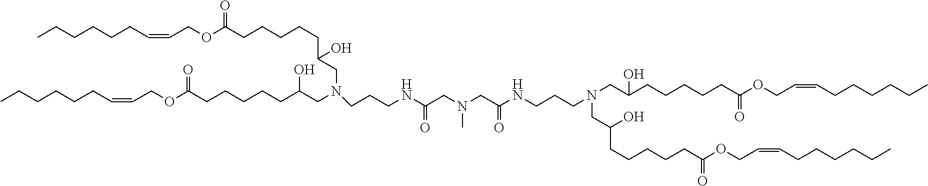 [SMILES: CCCCCC/C=C\COC(=O)CCCCCC(O)CN(CCCNC(=O)CN(C)CC(=O)NCCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC] |
| LXXXIII | 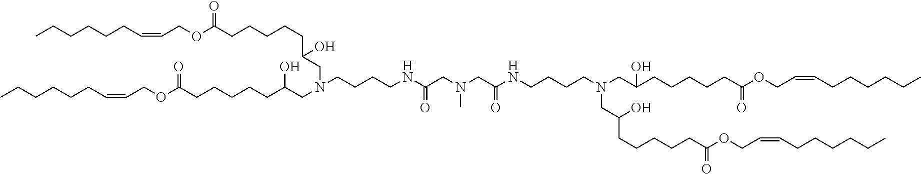 [SMILES: CCCCCC/C=C\COC(=O)CCCCCC(O)CN(CCCCNC(=O)CN(C)CC(=O)NCCCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC] |
| CVI | 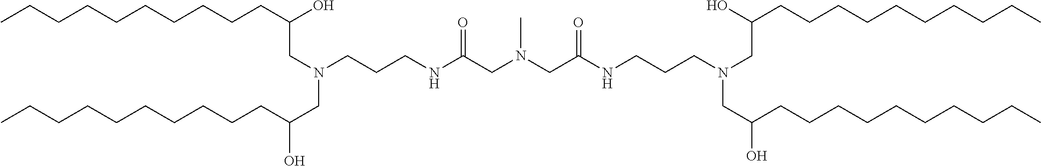 [SMILES: CCCCCCCCCCC(O)CN(CCCNC(=O)CN(C)CC(=O)NCCCN(CC(O)CCCCCCCCCC)CC(O)CCCCCCCCCC)CC(O)CCCCCCCCCC] |

Formula (IA1iii): 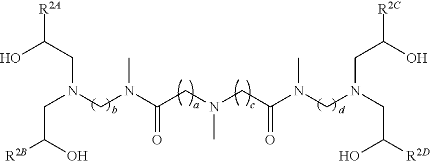 For example, wherein b is 3 or 4 and d is 3 or 4
Formula (IA1iiia): 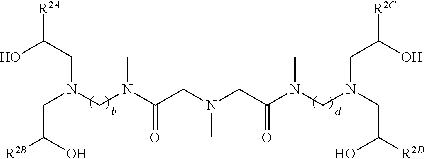 For example, wherein b is 3 or 4 and d is 3 or 4

| | |
|---|---|
| LXXVII | 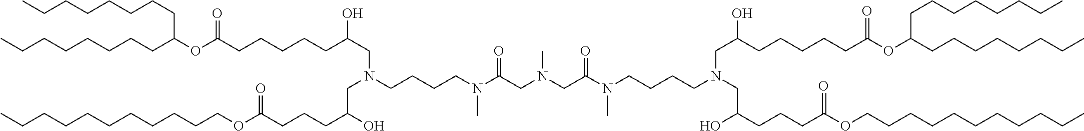 [SMILES: CCCCCCCCCCCOC(=O)CCCC(O)CN(CCCCN(C)C(=O)CN(C)CC(=O)N(C)CCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| LXXX | 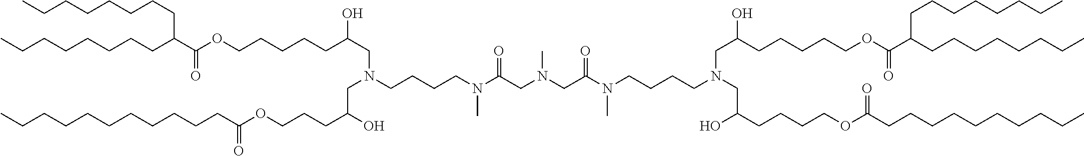 [SMILES: CCCCCCCCCCCC(=O)OCCCC(O)CN(CCCCN(C)C(=O)CN(C)CC(=O)N(C)CCCCN(CC(O)CCCCCOC(=O)C(CCCCCCCC)CCCCCCCC)CC(O)CCCCOC(=O)CCCCCCCCCC)CC(O)CCCCCOC(=O)C(CCCCCCCC)CCCCCCCC] |
| LXXXII | 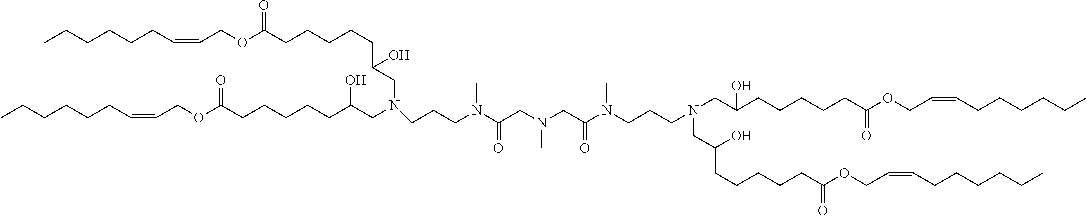 [SMILES: CCCCCC/C=C\COC(=O)CCCCCC(O)CN(CCCN(C)C(=O)CN(C)CC(=O)N(C)CCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC] |
| CVII | 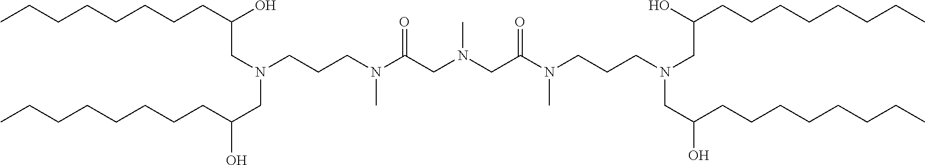 [SMILES: CCCCCCCCC(O)CN(CCCN(C)C(=O)CN(C)CC(=O)N(C)CCCN(CC(O)CCCCCCCC)CC(O)CCCCCCCC)CC(O)CCCCCCCC] |

Formula (IIC1): 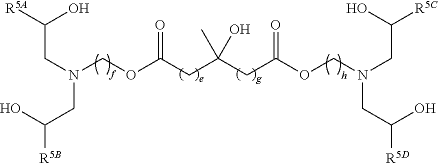 For example, wherein f is 3 and h is 3
Formula (IIC1ii): 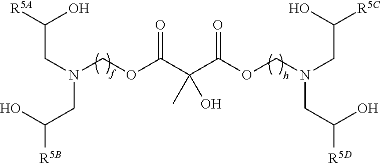 For example, wherein f is 3 and h is 3.

| | |
|---|---|
| LXXXV | 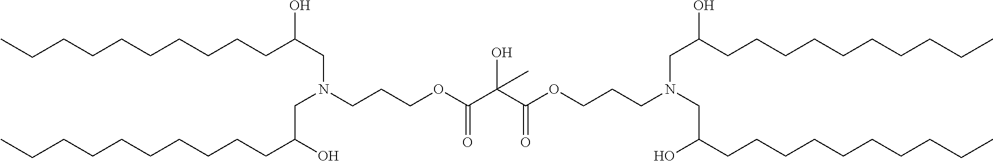 [SMILES: CCCCCCCCCCC(O)CN(CCCOC(=O)C(C)(O)C(=O)OCCCN(CC(O)CCCCCCCCCC)CC(O)CCCCCCCCCC)CC(O)CCCCCCCCCC] |

Formula (IIC2): 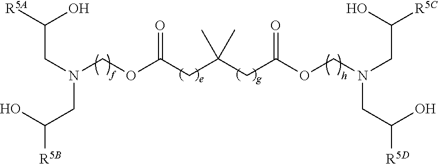 For example, wherein f is 4 and h is 4.
Formula (IIC2i): 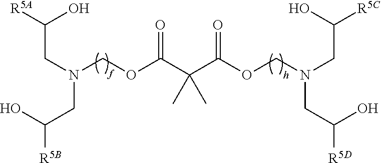 For example, wherein f is 4 and h is 4

| | |
|---|---|
| LXXXIV | 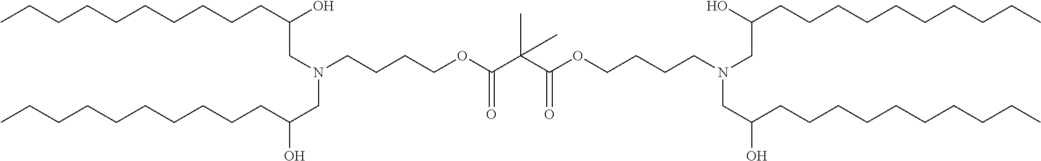 [SMILES: CCCCCCCCCCC(O)CN(CCCCOC(=O)C(C)(C)C(=O)OCCCCN(CC(O)CCCCCCCCCC)CC(O)CCCCCCCCCC)CC(O)CCCCCCCCCC] |

Formula (IIE): 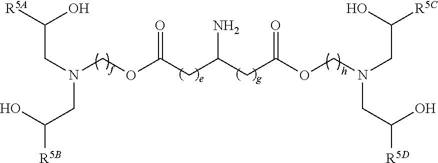 For example, wherein f is 3 and h is 3
Formula (IIE1): 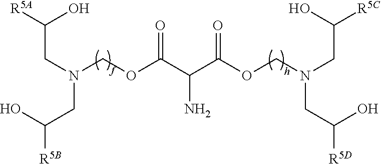 For example, wherein f is 3 and h is 3

| | |
|---|---|
| LXXXVI | 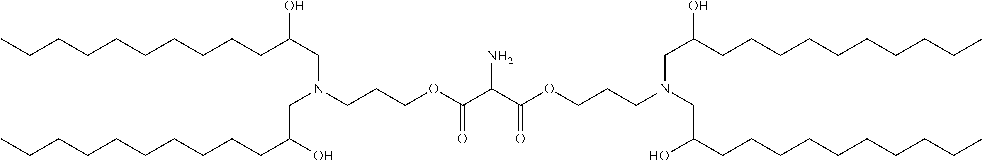 [SMILES: CCCCCCCCCCC(O)CN(CCCOC(=O)C(N)C(=O)OCCCN(CC(O)CCCCCCCCCC)CC(O)CCCCCCCCCC)CC(O)CCCCCCCCCC] |

Formula (IIIA): 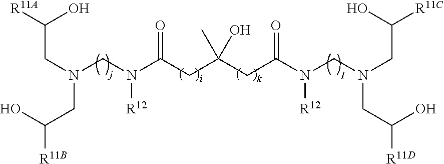 For example, wherein j is 3 or 4 and l is 3 or 4
Formula (IIIA1): 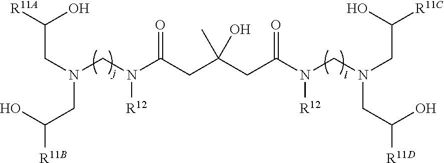 For example, wherein j is 3 or 4 and l is 3 or 4

| | |
|---|---|
| LXXXVIII | 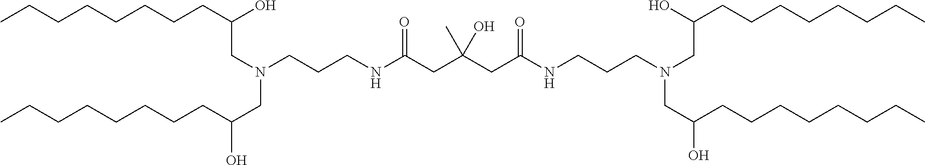 [SMILES: CCCCCCCCC(O)CN(CCCNC(=O)CC(C)(O)CC(=O)NCCCN(CC(O)CCCCCCCC)CC(O)CCCCCCCC)CC(O)CCCCCCCC] |
| LXXXIX | 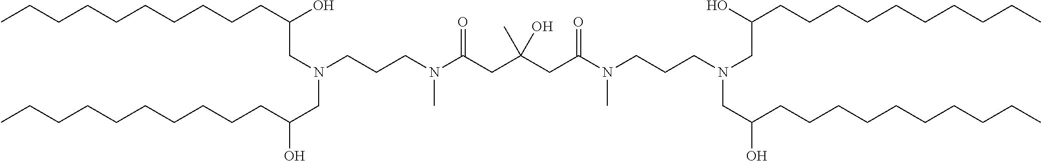 [SMILES: CCCCCCCCCCC(O)CN(CCCN(C)C(=O)CC(C)(O)CC(=O)N(C)CCCN(CC(O)CCCCCCCCCC)CC(O)CCCCCCCCCC)CC(O)CCCCCCCCCC] |
| XC | 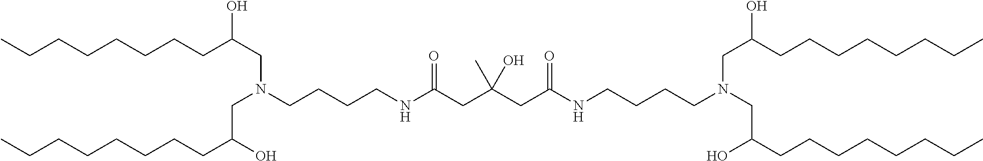 [SMILES: CCCCCCCCC(O)CN(CCCCNC(=O)CC(C)(O)CC(=O)NCCCCN(CC(O)CCCCCCCC)CC(O)CCCCCCCC)CC(O)CCCCCCCC] |
| XCI | 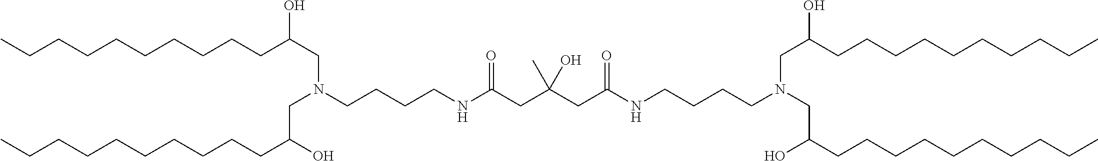 [SMILES: CCCCCCCCCCC(O)CN(CCCCNC(=O)CC(C)(O)CC(=O)NCCCCN(CC(O)CCCCCCCCCC)CC(O)CCCCCCCCCC)CC(O)CCCCCCCCCC] |
| XCII | 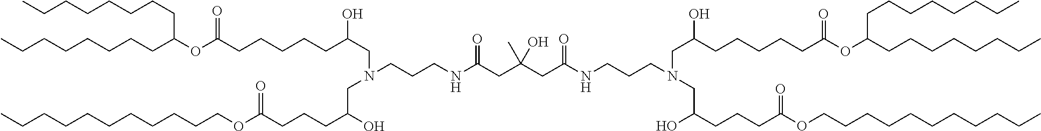 [SMILES: CCCCCCCCCCCOC(=O)CCCC(O)CN(CCCNC(=O)CC(C)(O)CC(=O)NCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| XCIII | 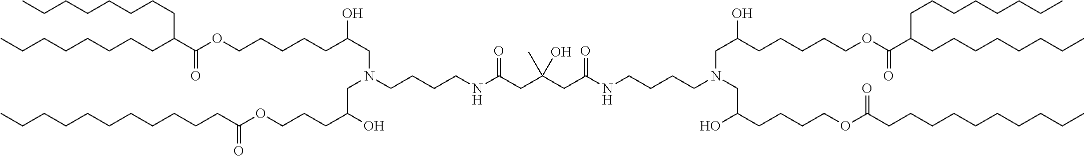 [SMILES: CCCCCCCCCCCC(=O)OCCCC(O)CN(CCCCNC(=O)CC(C)(O)CC(=O)NCCCCN(CC(O)CCCCCOC(=O)C(CCCCCCCC)CCCCCCCC)CC(O)CCCCOC(=O)CCCCCCCCCC)CC(O)CCCCCOC(=O)C(CCCCCCCC)CCCCCCCC] |
| XCIV | 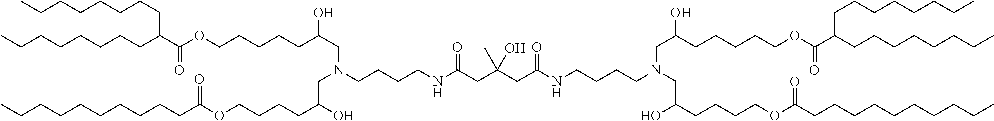 [SMILES: CCCCCCCCCCC(=O)OCCCCC(O)CN(CCCCNC(=O)CC(C)(O)CC(=O)NCCCCN(CC(O)CCCCCOC(=O)C(CCCCCCCC)CCCCCCCC)CC(O)CCCCOC(=O)CCCCCCCCCC)CC(O)CCCCCOC(=O)C(CCCCCCCC)CCCCCCCC] |
| CIV | 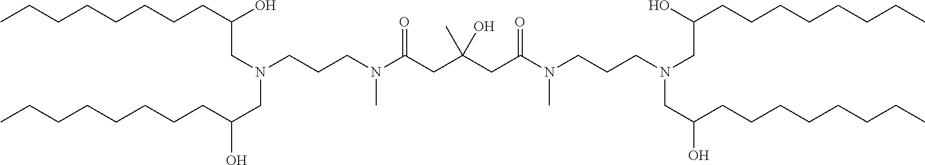 [SMILES: CCCCCCCCC(O)CN(CCCN(C)C(=O)CC(C)(O)CC(=O)N(C)CCCN(CC(O)CCCCCCCC)CC(O)CCCCCCCC)CC(O)CCCCCCCC] |
| CV | 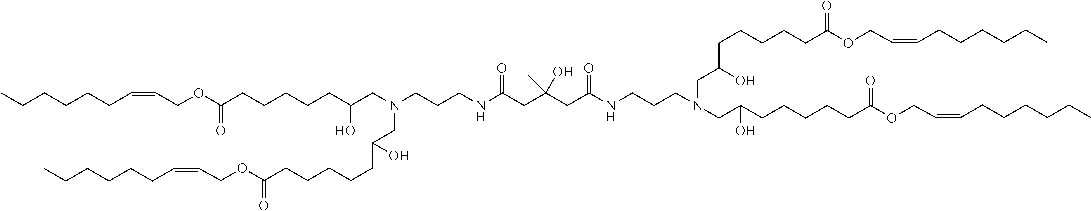 [SMILES: CCCCCC/C=C\COC(=O)CCCCCC(O)CN(CCCNC(=O)CC(C)(O)CC(=O)NCCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC] |

Formula (IIIB): 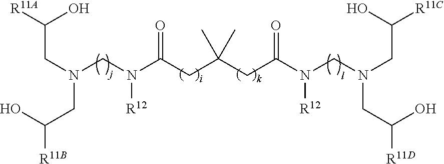 For example, wherein j is 3 and l is 3
Formula (IIIB1): 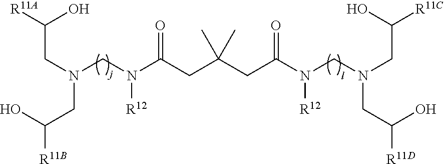 For example, wherein j is 3 and l is 3

| | |
|---|---|
| LXXXVII | 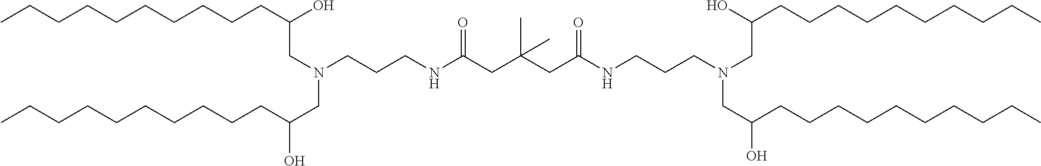 [SMILES: CCCCCCCCCCC(O)CN(CCCNC(=O)CC(C)(C)CC(=O)NCCCN(CC(O)CCCCCCCCCC)CC(O)CCCCCCCCCC)CC(O)CCCCCCCCCC] |

Formula (IVA): 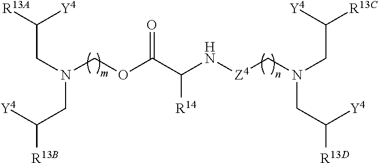 For example, wherein m is 3 and n is 3
Formula (IVA1): 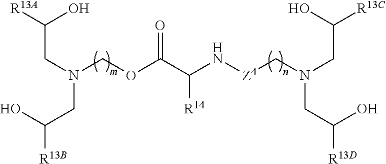 For example, wherein m is 3 and n is 3

| | |
|---|---|
| XCV | 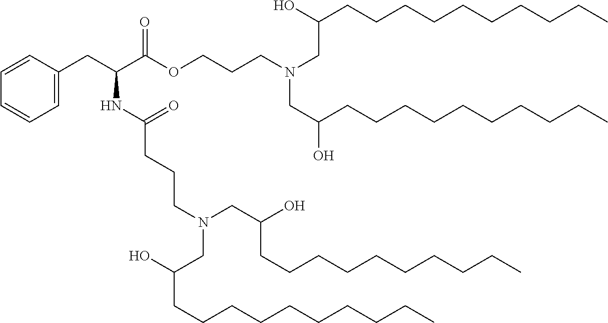 [SMILES: CCCCCCCCCCC(O)CN(CCCOC(=O)[C@H](Cc1ccccc1)NC(=O)CCCN(CC(O)CCCCCCCCCC)CC(O)CCCCCCCCCC)CC(O)CCCCCCCCCC] |
| XCVI | 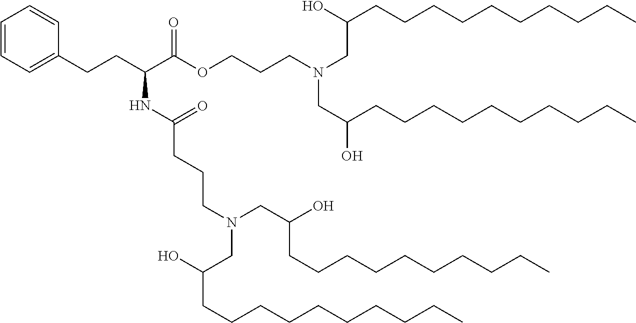 [SMILES: CCCCCCCCCCC(O)CN(CCCOC(=O)[C@H](CCc1ccccc1)NC(=O)CCCN(CC(O)CCCCCCCCCC)CC(O)CCCCCCCCCC)CC(O)CCCCCCCCCC] |
| XCVII | 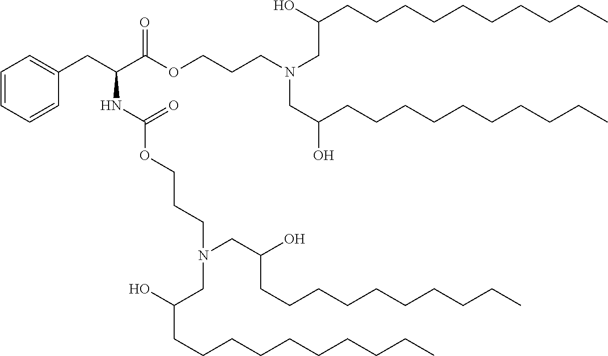 [SMILES: CCCCCCCCCCC(O)CN(CCCOC(=O)N[C@@H](Cc1ccccc1)C(=O)OCCCN(CC(O)CCCCCCCCCC)CC(O)CCCCCCCCCC)CC(O)CCCCCCCCCC] |
| XCVIII | 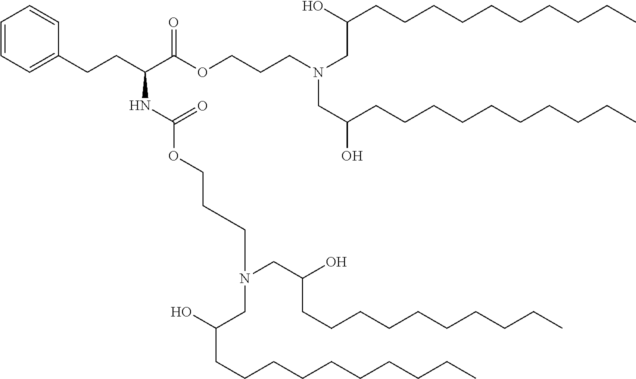 [SMILES: CCCCCCCCCCC(O)CN(CCCOC(=O)N[C@@H](CCc1ccccc1)C(=O)OCCCN(CC(O)CCCCCCCCCC)CC(O)CCCCCCCCCC)CC(O)CCCCCCCCCC] |
| XCIX | 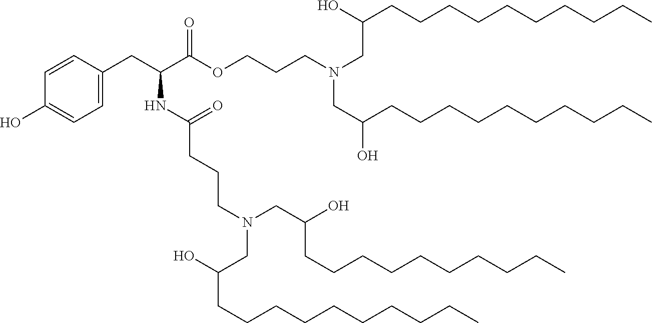 [SMILES: CCCCCCCCCCC(O)CN(CCCOC(=O)[C@H](Cc1ccc(O)cc1)NC(=O)CCCN(CC(O)CCCCCCCCCC)CC(O)CCCCCCCCCC)CC(O)CCCCCCCCCC] |
| C | 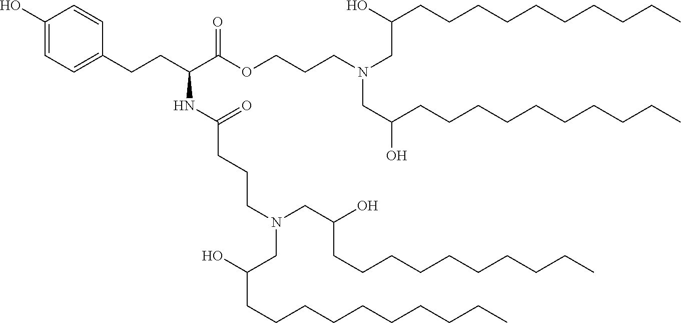 [SMILES: CCCCCCCCCCC(O)CN(CCCOC(=O)[C@H](CCc1ccc(O)cc1)NC(=O)CCCN(CC(O)CCCCCCCCCC)CC(O)CCCCCCCCCC)CC(O)CCCCCCCCCC] |
| CI | 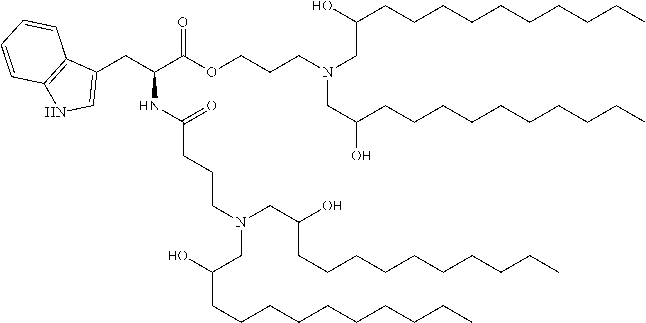 [SMILES: CCCCCCCCCCC(O)CN(CCCOC(=O)[C@H](Cc1c[nH]c2ccccc12)NC(=O)CCCN(CC(O)CCCCCCCCCC)CC(O)CCCCCCCCCC)CC(O)CCCCCCCCCC] |
| CII | 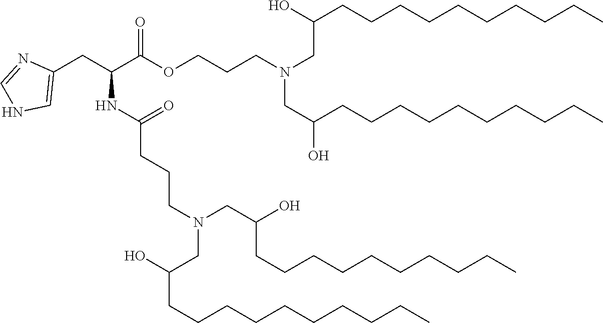 [SMILES: CCCCCCCCCCC(O)CN(CCCOC(=O)[C@H](Cc1c[nH]cn1)NC(=O)CCCN(CC(O)CCCCCCCCCC)CC(O)CCCCCCCCCC)CC(O)CCCCCCCCCC] |
| CIII | 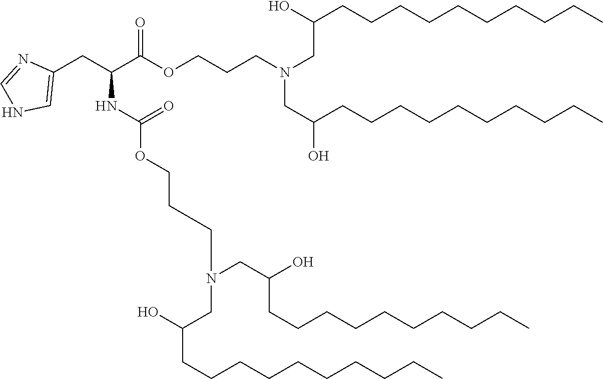 [SMILES: CCCCCCCCCCC(O)CN(CCCOC(=O)N[C@@H](Cc1c[nH]cn1)C(=O)OCCCN(CC(O)CCCCCCCCCC)CC(O)CCCCCCCCCC)CC(O)CCCCCCCCCC] |
| CVIII | 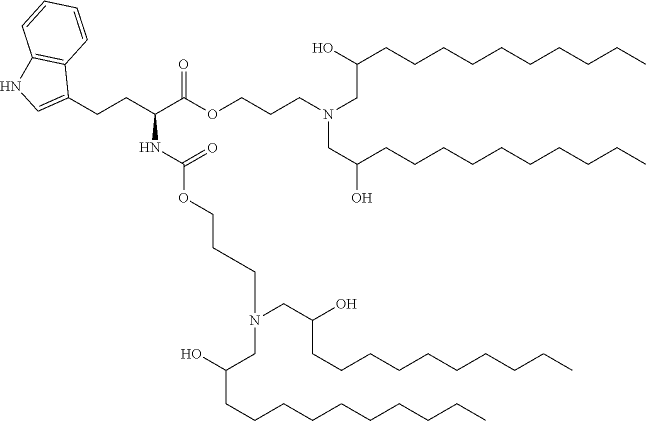 [SMILES: CCCCCCCCCCC(O)CN(CCCOC(=O)N[C@@H](CCc1c[nH]c2ccccc12)C(=O)OCCCN(CC(O)CCCCCCCCCC)CC(O)CCCCCCCCCC)CC(O)CCCCCCCCCC] |
| CIX | 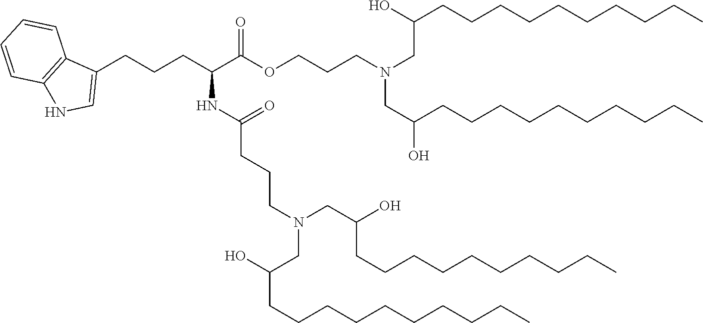 [SMILES: CCCCCCCCCCC(O)CN(CCCOC(=O)[C@H](CCCc1c[nH]c2ccccc12)NC(=O)CCCN(CC(O)CCCCCCCCCC)CC(O)CCCCCCCCCC)CC(O)CCCCCCCCCC] |
| CX | 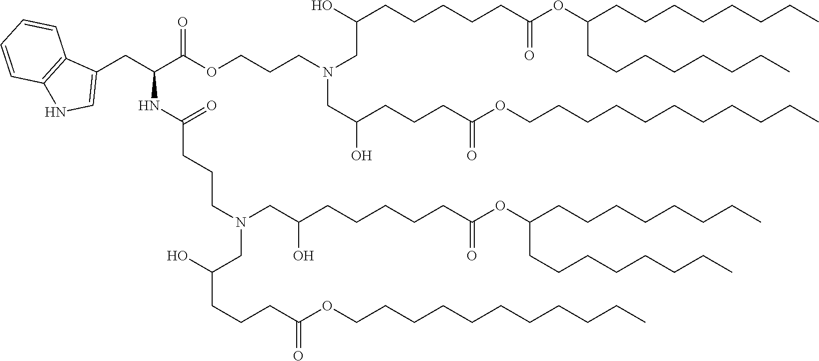 [SMILES: CCCCCCCCCCCOC(=O)CCCC(O)CN(CCCOC(=O)[C@H](Cc1c[nH]c2ccccc12)NC(=O)CCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| CXI | 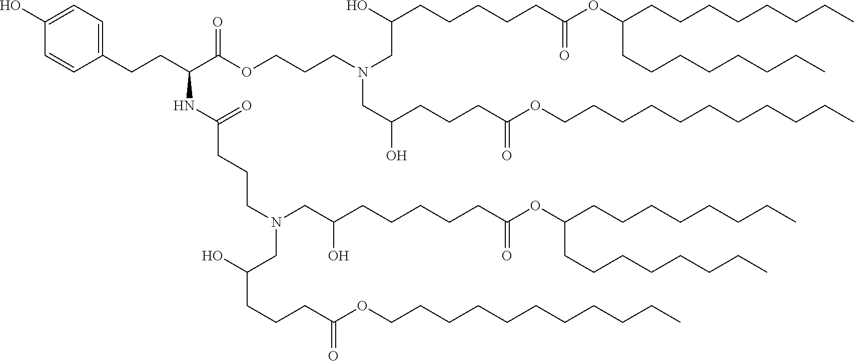 [SMILES: CCCCCCCCCCCOC(=O)CCCC(O)CN(CCCOC(=O)[C@H](CCc1ccc(O)cc1)NC(=O)CCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |

Formula (IVA2) 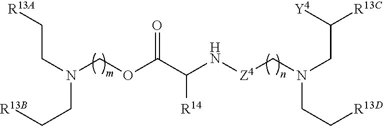 For example, wherein m is 3 and n is 3

| | |
|---|---|
| CXII | 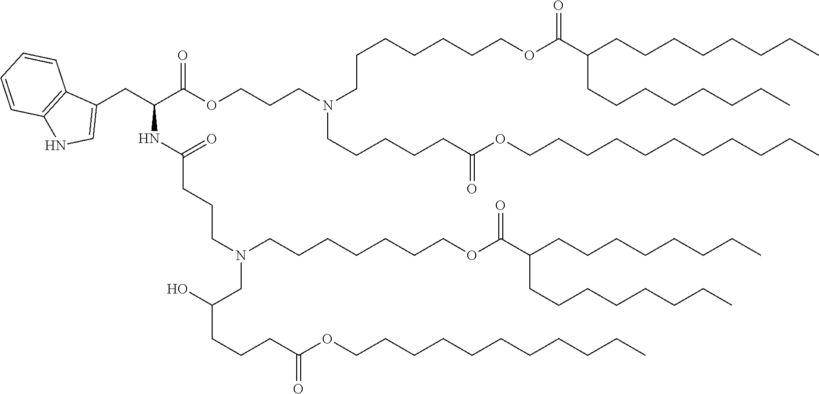 [SMILES: CCCCCCCCCCCOC(=O)CCCCCN(CCCCCCCOC(=O)C(CCCCCCCC)CCCCCCCC)CCCOC(=O)[C@H](Cc1c[nH]c2ccccc12)NC(=O)CCCN(CCCCCCCOC(=O)C(CCCCCCCC)CCCCCCCC)CC(O)CCCC(=O)OCCCCCCCCCCC] |
| CXIII | 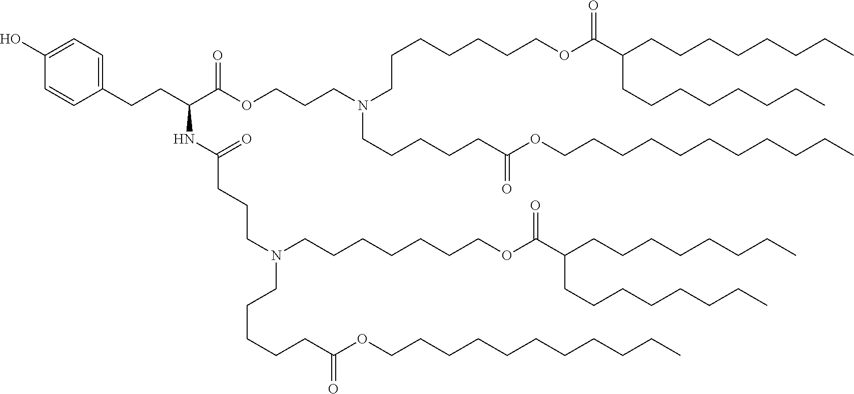 [SMILES: CCCCCCCCCCCOC(=O)CCCCCN(CCCCCCCOC(=O)C(CCCCCCCC)CCCCCCCC)CCCOC(=O)[C@H](CCc1ccc(O)cc1)NC(=O)CCCN(CCCCCCCOC(=O)C(CCCCCCCC)CCCCCCCC)CCCCCC(=O)OCCCCCCCCCCC] |
| CXIV | 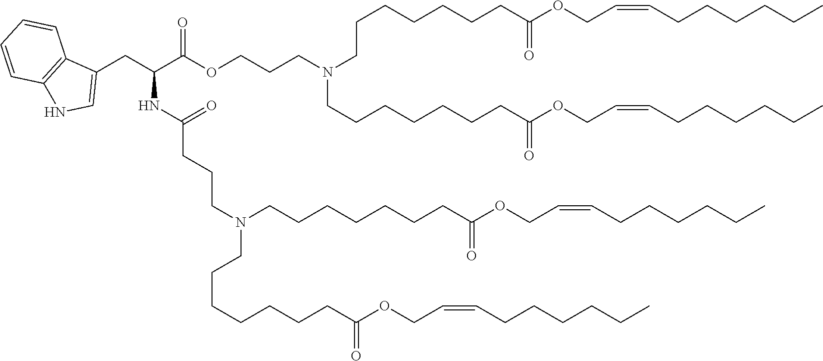 [SMILES: CCCCCC/C=C\COC(=O)CCCCCCCN(CCCCCCCC(=O)OC/C=C\CCCCCC)CCCOC(=O)[C@H](Cc1c[nH]c2ccccc12)NC(=O)CCCN(CCCCCCCC(=O)OC/C=C\CCCCCC)CCCCCCCC(=O)OC/C=C\CCCCCC] |
| CXV | 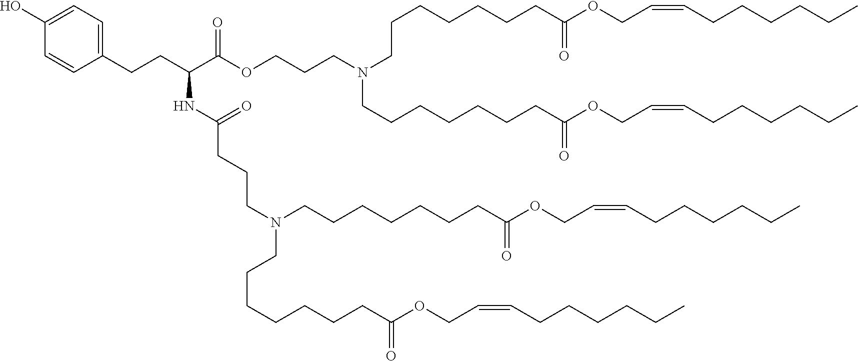 [SMILES: CCCCCC/C=C\COC(=O)CCCCCCCN(CCCCCCCC(=O)OC/C=C\CCCCCC)CCCOC(=O)[C@H](CCc1ccc(O)cc1)NC(=O)CCCN(CCCCCCCC(=O)OC/C=C\CCCCCC)CCCCCCCC(=O)OC/C=C\CCCCCC] |

Any of the compounds identified in Table B above may be provided in the form of a pharmaceutically acceptable salt and such salts are intended to be encompassed by the present invention.

The compounds of the invention as described herein can be prepared according to methods known in the art, including the exemplary syntheses of the Examples provided herein.

### Nucleic Acids

The compounds of the invention as described herein can be used to prepare compositions useful for the delivery of nucleic acids.

### Synthesis of Nucleic Acids

Nucleic acids according to the present invention may be synthesized according to any known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, mutated T7 or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7, mutated T7 or SP6 promoter, for in vitro transcription, followed by desired nucleotide sequence for desired mRNA and a termination signal.

Desired mRNA sequence(s) according to the invention may be determined and incorporated into a DNA template using standard methods. For example, starting from a desired amino acid sequence (e.g., an enzyme sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

Modified mRNA

In some embodiments, mRNA according to the present invention may be synthesized as unmodified or modified mRNA. Modified mRNA comprises nucleotide modifications in the RNA. A modified mRNA according to the invention can thus include nucleotide modification that are, for example, backbone modifications, sugar modifications or base modifications. In some embodiments, mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queuosine, beta-D-mannosyl-queuosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

### Pharmaceutical Formulations of Cationic Lipids and Nucleic Acids

In certain embodiments, the compounds of the invention as described herein, as well as pharmaceutical and liposomal compositions comprising such lipids, can be used in formulations to facilitate the delivery of encapsulated materials (e.g., one or more polynucleotides such as mRNA) to, and subsequent transfection of one or more target cells. For example, in certain embodiments cationic lipids described herein (and compositions such as liposomal compositions comprising such lipids) are characterized as resulting in one or more of receptor-mediated endocytosis, clathrin-mediated and caveolae-mediated endocytosis, phagocytosis and macropinocytosis, fusogenicity, endosomal or lysosomal disruption and/or releasable properties that afford such compounds advantages relative other similarly classified lipids.

According to the present invention, a nucleic acid, e.g., mRNA encoding a protein (e.g., a full length, fragment or portion of a protein) as described herein may be delivered via a delivery vehicle comprising a compound of the invention as described herein.

As used herein, the terms “delivery vehicle,” “transfer vehicle,” “nanoparticle,” or grammatical equivalents thereof, are used interchangeably.

For example, the present invention provides a composition (e.g., a pharmaceutical composition) comprising a compound described herein and one or more polynucleotides. A composition (e.g., a pharmaceutical composition) may further comprise one or more cationic lipids, one or more non-cationic lipids, one or more cholesterol-based lipids and/or one or more PEG-modified lipids.

In certain embodiments a composition exhibits an enhanced (e.g., increased) ability to transfect one or more target cells. Accordingly, also provided herein are methods of transfecting one or more target cells. Such methods generally comprise the step of contacting the one or more target cells with the cationic lipids and/or pharmaceutical compositions disclosed herein (e.g., a liposomal formulation comprising a compound described herein encapsulating one or more polynucleotides) such that the one or more target cells are transfected with the materials encapsulated therein (e.g., one or more polynucleotides). As used herein, the terms “transfect” or “transfection” refer to the intracellular introduction of one or more encapsulated materials (e.g., nucleic acids and/or polynucleotides) into a cell (e.g., into a target cell). The introduced polynucleotide may be stably or transiently maintained in the target cell. The term “transfection efficiency” refers to the relative amount of such encapsulated material (e.g., polynucleotides) up-taken by, introduced into, and/or expressed by the target cell which is subject to transfection. In practice, transfection efficiency may be estimated by the amount of a reporter polynucleotide product produced by the target cells following transfection. In certain embodiments, the compounds and pharmaceutical compositions described herein demonstrate high transfection efficiencies thereby improving the likelihood that appropriate dosages of the encapsulated materials (e.g., one or more polynucleotides) will be delivered to the site of pathology and subsequently expressed, while at the same time minimizing potential systemic adverse effects or toxicity associated with the compound or their encapsulated contents.

Following transfection of one or more target cells by, for example, the polynucleotides encapsulated in the one or more lipid nanoparticles comprising the pharmaceutical or liposomal compositions disclosed herein, the production of the product (e.g., a polypeptide or protein) encoded by such polynucleotide may be stimulated and the capability of such target cells to express the polynucleotide and produce, for example, a polypeptide or protein of interest is enhanced. For example, transfection of a target cell by one or more compounds or pharmaceutical compositions encapsulating mRNA will enhance (i.e., increase) the production of the protein or enzyme encoded by such mRNA.

Further, delivery vehicles described herein (e.g., liposomal delivery vehicles) may be prepared to preferentially distribute to other target tissues, cells or organs, such as the heart, lungs, kidneys, spleen. In embodiments, the lipid nanoparticles of the present invention may be prepared to achieve enhanced delivery to the target cells and tissues. For example, polynucleotides (e.g., mRNA) encapsulated in one or more of the compounds or pharmaceutical and liposomal compositions described herein can be delivered to and/or transfect targeted cells or tissues. In some embodiments, the encapsulated polynucleotides (e.g., mRNA) are capable of being expressed and functional polypeptide products produced (and in some instances excreted) by the target cell, thereby conferring a beneficial property to, for example the target cells or tissues. Such encapsulated polynucleotides (e.g., mRNA) may encode, for example, a hormone, enzyme, receptor, polypeptide, peptide or other protein of interest.

### Liposomal Delivery Vehicles

In some embodiments, a composition is a suitable delivery vehicle. In embodiments, a composition is a liposomal delivery vehicle, e.g., a lipid nanoparticle.

The terms “liposomal delivery vehicle” and “liposomal composition” are used interchangeably.

Enriching liposomal compositions with one or more of the cationic lipids disclosed herein may be used as a means of improving the safety profile or otherwise conferring one or more desired properties to such enriched liposomal composition (e.g., improved delivery of the encapsulated polynucleotides to one or more target cells and/or reduced in vivo toxicity of a liposomal composition). Accordingly, also contemplated are pharmaceutical compositions, and in particular liposomal compositions, that comprise one or more of the cationic lipids disclosed herein.

Thus, in certain embodiments, the compounds of the invention as described herein may be used as a component of a liposomal composition to facilitate or enhance the delivery and release of encapsulated materials (e.g., one or more therapeutic agents) to one or more target cells (e.g., by permeating or fusing with the lipid membranes of such target cells).

As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphophilic polymers and surfactants (e.g., polymerosomes, niosomes, etc.). In the context of the present invention, a liposomal delivery vehicle typically serves to transport a desired mRNA to a target cell or tissue.

In certain embodiments, such compositions (e.g., liposomal compositions) are loaded with or otherwise encapsulate materials, such as for example, one or more biologically-active polynucleotides (e.g., mRNA).

In embodiments, a composition (e.g., a pharmaceutical composition) comprises an mRNA encoding a protein, encapsulated within a liposome. In embodiments, a liposome comprises one or more cationic lipids, one or more non-cationic lipids, one or more cholesterol-based lipids and one or more PEG-modified lipids, and wherein at least one cationic lipid is a compound of the invention as described herein. In embodiments, a composition comprises an mRNA encoding for a peptide or protein (e.g., any peptide or protein described herein). In embodiments, a composition comprises an mRNA encoding for a peptide (e.g., any peptide described herein). In embodiments, a composition comprises an mRNA encoding for a protein (e.g., any protein described herein).

In embodiments, a composition (e.g., a pharmaceutical composition) comprises a nucleic acid encapsulated within a liposome, wherein the liposome comprises a compound described herein.

In embodiments, a nucleic acid is an mRNA encoding a peptide or protein. In embodiments, an mRNA encodes a peptide or protein for use in the delivery to or treatment of the lung of a subject or a lung cell. In embodiments, an mRNA encodes a peptide or protein for use in the delivery to or treatment of the liver of a subject or a liver cell. Still other exemplary mRNAs are described herein.

In embodiments, a liposomal delivery vehicle (e.g., a lipid nanoparticle) can have a net positive charge.

In embodiments, a liposomal delivery vehicle (e.g., a lipid nanoparticle) can have a net negative charge.

In embodiments, a liposomal delivery vehicle (e.g., a lipid nanoparticle) can have a net neutral charge.

In embodiments, a lipid nanoparticle that encapsulates a nucleic acid (e.g., mRNA encoding a peptide or protein) comprises one or more compounds of the invention as described herein.

For example, the amount of a compound of the invention as described herein in a composition can be described as a percentage (“wt %”) of the combined dry weight of all lipids of a composition (e.g., the combined dry weight of all lipids present in a liposomal composition).

In embodiments of the pharmaceutical compositions described herein, a compound of the invention as described herein is present in an amount that is about 0.5 wt % to about 30 wt % (e.g., about 0.5 wt % to about 20 wt %) of the combined dry weight of all lipids present in a composition (e.g., a liposomal composition).

In embodiments, a compound of the invention as described herein is present in an amount that is about 1 wt % to about 30 wt %, about 1 wt % to about 20 wt %, about 1 wt % to about 15 wt %, about 1 wt % to about 10 wt %, or about 5 wt % to about 25 wt % of the combined dry weight of all lipids present in a composition (e.g., a liposomal composition).

In embodiments, a compound of the invention as described herein is present in an amount that is about 0.5 wt % to about 5 wt %, about 1 wt % to about 10 wt %, about 5 wt % to about 20 wt %, or about 10 wt % to about 20 wt % of the combined dry weight of all lipids present in a composition such as a liposomal delivery vehicle.

In embodiments, the amount of a compound of the invention as described herein is present in an amount that is at least about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the combined dry weight of total lipids in a composition (e.g., a liposomal composition).

In embodiments, the amount of a compound of the invention as described herein is present in an amount that is no more than about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the combined dry weight of total lipids in a composition (e.g., a liposomal composition).

In embodiments, a composition (e.g., a liposomal delivery vehicle such as a lipid nanoparticle) comprises about 0.1 wt % to about 20 wt % (e.g., about 0.1 wt % to about 15 wt %) of a compound described herein. In embodiments, a delivery vehicle (e.g., a liposomal delivery vehicle such as a lipid nanoparticle) comprises about 0.5 wt %, about 1 wt %, about 3 wt %, about 5 wt %, or about 10 wt % of a compound described herein. In embodiments, a delivery vehicle (e.g., a liposomal delivery vehicle such as a lipid nanoparticle) comprises up to about 0.5 wt %, about 1 wt %, about 3 wt %, about 5 wt %, about 10 wt %, about 15 wt %, or about 20 wt % of a compound described herein. In embodiments, the percentage results in an improved beneficial effect (e.g., improved delivery to targeted tissues such as the liver or the lung).

The amount of a compound of the invention as described herein in a composition also can be described as a percentage (“mol %”) of the combined molar amounts of total lipids of a composition (e.g., the combined molar amounts of all lipids present in a liposomal delivery vehicle).

In embodiments of pharmaceutical compositions described herein, a compound of the invention as described herein is present in an amount that is about 0.5 mol % to about 50 mol % (e.g., about 0.5 mol % to about 20 mol %) of the combined molar amounts of all lipids present in a composition such as a liposomal delivery vehicle.

In embodiments, a compound of the invention as described herein is present in an amount that is about 0.5 mol % to about 5 mol %, about 1 mol % to about 10 mol %, about 5 mol % to about 20 mol %, about 10 mol % to about 20 mol %, about 15 mol % to about 30 mol %, about 20 mol % to about 35 mol %, about 25 mol % to about 40 mol %, about 30 mol % to about 45 mol %, about 35 mol % to about 50 mol %, about 40 mol % to about 55 mol %, or about 45 mol % to about 60 mol % of the combined molar amounts of all lipids present in a composition such as a liposomal delivery vehicle. In embodiments, a compound of the invention as described herein is present in an amount that is about 1 mol % to about 60 mol %, 1 mol % to about 50 mol %, 1 mol % to about 40 mol %, 1 mol % to about 30 mol %, about 1 mol % to about 20 mol %, about 1 mol % to about 15 mol %, about 1 mol % to about 10 mol %, about 5 mol % to about 55 mol %, about 5 mol % to about 45 mol %, about 5 mol % to about 35 mol % or about 5 mol % to about 25 mol % of the combined molar amounts of all lipids present in a composition such as a liposomal delivery vehicle

In certain embodiments, a compound of the invention as described herein can comprise from about 0.1 mol % to about 50 mol %, or from 0.5 mol % to about 50 mol %, or from about 1 mol % to about 50 mol %, or from about 5 mol % to about 50 mol %, or from about 10 mol % to about 50 mol %, or from about 15 mol % to about 50 mol %, or from about 20 mol % to about 50 mol %, or from about 25 mol % to about 50 mol %, or from about 30 mol % to about 50 mol %, of the total amount of lipids in a composition (e.g., a liposomal delivery vehicle).

In certain embodiments, a compound of the invention as described herein can comprise greater than about 0.1 mol %, or greater than about 0.5 mol %, or greater than about 1 mol %, greater than about 5 mol %, greater than about 10 mol %, greater than about 20 mol %, greater than about 30 mol %, or greater than about 40 mol % of the total amount of lipids in the lipid nanoparticle.

In certain embodiments, a compound as described can comprise less than about 60 mol %, or less than about 55 mol %, or less than about 50 mol %, or less than about 45 mol %, or less than about 40 mol %, or less than about 35 mol %, less than about 30 mol %, or less than about 25 mol %, or less than about 10 mol %, or less than about 5 mol %, or less than about 1 mol % of the total amount of lipids in a composition (e.g., a liposomal delivery vehicle).

In embodiments, the amount of a compound of the invention as described herein is present in an amount that is at least about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, about 96 mol %, about 97 mol %, about 98 mol %, or about 99 mol % of the combined molar amounts of total lipids in a composition (e.g., a liposomal composition).

In embodiments, the amount of a compound of the invention as described herein is present in an amount that is no more than about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, about 96 mol %, about 97 mol %, about 98 mol %, or about 99 mol % of the combined molar amounts of total lipids in a composition (e.g., a liposomal composition).

In embodiments, the percentage results in an improved beneficial effect (e.g., improved delivery to targeted tissues such as the liver or the lung).

In a typical embodiment, a composition of the invention (e.g., a liposomal composition) comprises one or more cationic lipids, one or more non-cationic lipids, one or more cholesterol-based lipids, and one or more PEG-modified lipids, wherein at least one cationic lipid is a compound of the invention as described herein. For example, a composition suitable for practicing the invention has four lipid components comprising a compound of the invention as described herein as the cationic lipid component, a non-cationic lipid, a cholesterol-based lipid, and a PEG-modified lipid. The non-cationic lipid may be DOPE or DEPE. The cholesterol-based lipid may be cholesterol. The PEG-modified lipid may be DMG-PEG2K.

In further embodiments, pharmaceutical (e.g., liposomal) compositions comprise one or more of a PEG-modified lipid, a non-cationic lipid and a cholesterol lipid. In other embodiments, such pharmaceutical (e.g., liposomal) compositions comprise: one or more PEG-modified lipids; one or more non-cationic lipids; and one or more cholesterol lipids. In yet further embodiments, such pharmaceutical (e.g., liposomal) compositions comprise: one or more PEG-modified lipids and one or more cholesterol lipids.

In embodiments, a composition (e.g., lipid nanoparticle) that encapsulates a nucleic acid (e.g., mRNA encoding a peptide or protein) comprises one or more compounds of the invention as described herein, and one or more lipids selected from the group consisting of a cationic lipid, a non-cationic lipid, and a PEGylated lipid.

In embodiments, a composition (e.g., lipid nanoparticle) that encapsulates a nucleic acid (e.g., mRNA encoding a peptide or protein) comprises one or more compound of the invention as described herein; one or more lipids selected from the group consisting of a cationic lipid, a non-cationic lipid, and a PEGylated lipid; and further comprises a cholesterol-based lipid. Typically, such a composition has four lipid components comprising a compound of the invention as described herein as the cationic lipid component, a non-cationic lipid (e.g., DOPE), a cholesterol-based lipid (e.g., cholesterol) and a PEG-modified lipid (e.g., DMG-PEG2K).

In embodiments, a lipid nanoparticle that encapsulates a nucleic acid (e.g., mRNA encoding a peptide or protein) comprises one or more compounds of the invention as described herein, as well as one or more lipids selected from the group consisting of a cationic lipid, a non-cationic lipid, a PEGylated lipid, and a cholesterol-based lipid.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). Thus, the molar ratios may be adjusted accordingly.

### Cationic Lipids

In addition to any of the compounds of the invention as described herein, a composition may comprise one or more additional cationic lipids.

In some embodiments, liposomes may comprise one or more additional cationic lipids. As used herein, the phrase “cationic lipid” refers to any of a number of lipid species that have a net positive charge at a selected pH, such as physiological pH. Several cationic lipids have been described in the literature, many of which are commercially available.

Suitable additional cationic lipids for use in the compositions include the cationic lipids as described in the literature.

### Helper Lipids

Compositions (e.g., liposomal compositions) may also comprise one or more helper lipids. Such helper lipids include non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), 1,2-Dierucoyl-sn-glycero-3-phosphoethanolamine (DEPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof. A non-cationic or helper lipid suitable for practicing the invention is dioleoylphosphatidylethanolamine (DOPE). Alternatively, 1,2-Dierucoyl-sn-glycero-3-phosphoethanolamine (DEPE) can be used as a non-cationic or helper lipid.

In some embodiments, a non-cationic lipid is a neutral lipid, i.e., a lipid that does not carry a net charge in the conditions under which the composition is formulated and/or administered.

In some embodiments, a non-cationic lipid may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 mol %, greater than about 10 mol %, greater than about 20 mol %, greater than about 30 mol %, or greater than about 40 mol %. In some embodiments, the percentage total non-cationic lipids in a liposome may be greater than about 5 mol %, greater than about 10 mol %, greater than about 20 mol %, greater than about 30 mol %, or greater than about 40 mol %. In some embodiments, the percentage of non-cationic lipid in a liposome is no more than about 5 mol %, no more than about 10 mol %, no more than about 20 mol %, no more than about 30 mol %, or no more than about 40 mol %. In some embodiments, the percentage total non-cationic lipids in a liposome may be no more than about 5 mol %, no more than about 10 mol %, no more than about 20 mol %, no more than about 30 mol %, or no more than about 40 mol %.

In some embodiments, a non-cationic lipid may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 wt %, greater than about 10 wt %, greater than about 20 wt %, greater than about 30 wt %, or greater than about 40 wt %. In some embodiments, the percentage total non-cationic lipids in a liposome may be greater than about 5 wt %, greater than about 10 wt %, greater than about 20 wt %, greater than about 30 wt %, or greater than about 40 wt %. In some embodiments, the percentage of non-cationic lipid in a liposome is no more than about 5 wt %, no more than about 10 wt %, no more than about 20 wt %, no more than about 30 wt %, or no more than about 40 wt %. In some embodiments, the percentage total non-cationic lipids in a liposome may be no more than about 5 wt %, no more than about 10 wt %, no more than about 20 wt %, no more than about 30 wt %, or no more than about 40 wt %.

### Cholesterol-Based Lipids

In some embodiments, a composition (e.g., a liposomal composition) comprises one or more cholesterol-based lipids. For example, a suitable cholesterol-based lipid for practicing the invention is cholesterol. Other suitable cholesterol-based lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), beta-sitosterol, delta 5 avenasterol, or imidazole cholesterol ester (ICE), which has the following structure,

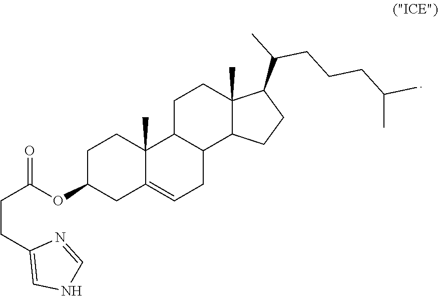 [SMILES: CC(C)CCCC(C)[C@H]1CCC2C3CC=C4C[C@@H](OC(=O)CCc5c[nH]cn5)CC[C@]4(C)C3CC[C@@]21C]

In some embodiments, a cholesterol-based lipid may be present in a molar ratio (mol %) of about 1% to about 30%, or about 5% to about 20% of the total lipids present in a liposome. In some embodiments, the percentage of cholesterol-based lipid in the lipid nanoparticle may be greater than about 5 mol %, greater than about 10 mol %, greater than about 20 mol %, greater than about 30 mol %, or greater than about 40 mol %. In some embodiments, the percentage of cholesterol-based lipid in the lipid nanoparticle may be no more than about 5 mol %, no more than about 10 mol %, no more than about 20 mol %, no more than about 30 mol %, or no more than about 40 mol %.

In some embodiments, a cholesterol-based lipid may be present in a weight ratio (wt %) of about 1% to about 30%, or about 5% to about 20% of the total lipids present in a liposome. In some embodiments, the percentage of cholesterol-based lipid in the lipid nanoparticle may be greater than about 5 wt %, greater than about 10 wt %, greater than about 20 wt %, greater than about 30 wt %, or greater than about 40 wt %. In some embodiments, the percentage of cholesterol-based lipid in the lipid nanoparticle may be no more than about 5 wt %, no more than about 10 wt %, no more than about 20 wt %, no more than about 30 wt %, or no more than about 40 wt %.

### PEGylated Lipids

In some embodiments, a composition (e.g., a liposomal composition) comprises one or more further PEGylated lipids. A suitable PEG-modified or PEGylated lipid for practicing the invention is 1,2-dimyristoyl-rac-glycero-3-methoxypolyethylene glycol-2000 (DMG-PEG2K).

For example, the use of polyethylene glycol (PEG)-modified phospholipids and derivatized lipids such as derivatized ceramides (PEG-CER), including N-octanoyl-sphingosine-1-[succinyl(methoxy polyethylene glycol)-2000](C8 PEG-2000 ceramide) is also contemplated by the present invention in combination with one or more of compounds of the invention as described herein and, in some embodiments, other lipids together which comprise the liposome. In some embodiments, particularly useful exchangeable lipids are PEG-ceramides having shorter acyl chains (e.g., C₁₄ or C₁₈).

Contemplated further PEG-modified lipids (also referred to herein as a PEGylated lipid, which term is interchangeable with PEG-modified lipid) include, but are not limited to, a polyethylene glycol chain of up to 5 kDa in length covalently attached to a lipid with alkyl chain(s) of C₆-C₂₀ length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613).

Further PEG-modified phospholipid and derivatized lipids of the present invention may be present in a molar ratio (mol %) from about 0% to about 10%, about 0.5% to about 10%, about 1% to about 10%, about 2% to about 10%, or about 3% to about 5% of the total lipid present in the composition (e.g., a liposomal composition).

### Pharmaceutical Formulations and Therapeutic Uses

Compounds of the invention as described herein may be used in the preparation of compositions (e.g., to construct liposomal compositions) that facilitate or enhance the delivery and release of encapsulated materials (e.g., one or more therapeutic polynucleotides) to one or more target cells (e.g., by permeating or fusing with the lipid membranes of such target cells).

For example, when a liposomal composition (e.g., a lipid nanoparticle) comprises or is otherwise enriched with one or more of the compounds disclosed herein, the phase transition in the lipid bilayer of the one or more target cells may facilitate the delivery of the encapsulated materials (e.g., one or more therapeutic polynucleotides encapsulated in a lipid nanoparticle) into the one or more target cells.

Similarly, in certain embodiments compounds of the invention as described herein may be used to prepare liposomal vehicles that are characterized by their reduced toxicity in vivo. In certain embodiments, the reduced toxicity is a function of the high transfection efficiencies associated with the compositions disclosed herein, such that a reduced quantity of such composition may be administered to the subject to achieve a desired therapeutic response or outcome.

Thus, pharmaceutical formulations comprising a compound described and nucleic acids provided by the present invention may be used for various therapeutic disease and/or disease prevention purposes. To facilitate delivery of nucleic acids in vivo, a compound described herein and nucleic acids can be formulated in combination with one or more additional pharmaceutical carriers, targeting ligands or stabilizing reagents. In some embodiments, a compound described herein can be formulated via pre-mixed lipid solution. In other embodiments, a composition comprising a compound described herein can be formulated using post-insertion techniques into the lipid membrane of the nanoparticles. Techniques for formulation and administration of drugs may be found in “Remington's Pharmaceutical Sciences,” Mack Publishing Co., Easton, Pa., latest edition.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments the administration results in delivery of the nucleic acids to a muscle cell. In some embodiments the administration results in delivery of the nucleic acids to a hepatocyte (i.e., liver cell).

A common route for administering a liposomal composition of the invention may be intravenous delivery, in particular when treating metabolic disorders, especially those affecting the liver (e.g., ornithine transcarbamylase (OTC) deficiency). Alternatively, depending on the disease or disorder to be treated, the liposomal composition may be administered via pulmonary delivery (e.g., for the treatment of cystic fibrosis). For vaccination, a liposomal composition of the invention is typically administered intramuscularly. Alternatively, a liposomal composition of the invention may be administered intranasally for vaccination. Diseases or disorders affecting the eye may be treated by administering a liposomal composition of the invention intravitreally.

Alternatively or additionally, pharmaceutical formulations of the invention may be administered in a local rather than systemic manner, for example, via injection of the pharmaceutical formulation directly into a targeted tissue (e.g., in a sustained release formulation). Local delivery can be affected in various ways, depending on the tissue to be targeted. Exemplary tissues in which mRNA may be delivered and/or expressed include, but are not limited to the liver, kidney, heart, spleen, serum, brain, skeletal muscle, lymph nodes, skin, and/or cerebrospinal fluid. In embodiments, the tissue to be targeted in the liver. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection.

Compositions described herein can comprise mRNA encoding peptides including those described herein (e.g., a polypeptide such as a protein).

In embodiments, a mRNA encodes a polypeptide.

In embodiments, a mRNA encodes a peptide. In embodiments, the peptide is an antigen.

In embodiments, a mRNA encodes a protein.

The present invention provides methods for delivering a composition having full-length mRNA molecules encoding a peptide or protein of interest for use in the treatment of a subject, e.g., a human subject or a cell of a human subject or a cell that is treated and delivered to a human subject.

### Delivery Methods

The route of delivery used in the methods of the invention allows for non-invasive, self-administration of the compounds of the invention. In some embodiments, the methods involve intranasal, intratracheal or pulmonary administration by aerosolization, nebulization, or instillation of a compositions comprising mRNA encoding a therapeutic peptide or protein in a suitable transfection or lipid carrier vehicles as described above. In some embodiments, the peptide or protein is encapsulated with a liposome. In some embodiments, the liposome comprises a lipid, which is a compound of the invention. As used herein below, administration of a compound of the invention includes administration of a composition comprising a compound of the invention.

Although the local cells and tissues of the lung represent a potential target capable of functioning as a biological depot or reservoir for production and secretion of the protein encoded by the mRNA, applicants have discovered that administration of the compounds of the invention to the lung via aerosolization, nebulization, or instillation results in the distribution of even non-secreted proteins outside the lung cells. Without wishing to be bound by any particular theory, it is contemplated that nanoparticle compositions of the invention pass, through the lung airway-blood barrier, resulting in translation of the intact nanoparticle to non-lung cells and tissues, such as, e.g., the heart, the liver, the spleen, where it results in the production of the encoded peptide or protein in these non-lung tissues. Thus, the utility of the compounds of the invention and methods of the invention extend beyond production of therapeutic protein in lung cells and tissues of the lung and can be used to delivery to non-lung target cells and/or tissues. They are useful in the management and treatment of a large number of diseases. In certain embodiments, the compounds of the invention, used in the methods of the invention result in the distribution of the mRNA encapsulated nanoparticles and production of the encoded peptide or protein in the liver, spleen, heart, and/or other non-lung cells. For example, administration of the compounds of the invention, by aerosolization, nebulization, or instillation to the lung will result in the composition itself and its peptide or protein product (e.g., an antigen or functional protein) will be detectable in both the local cells and tissues of the lung, as well as in peripheral target cells, tissues and organs as a result of translocation of the mRNA and delivery vehicle to non-lung cells.

In certain embodiments, the compounds of the invention may be employed in the methods of the invention to specifically target peripheral cells or tissues. Following the pulmonary delivery, it is contemplated the compounds of the invention cross the lung airway-blood barrier and distribute into cells other than the local lung cells. Accordingly, the compounds disclosed herein may be administered to a subject by way of the pulmonary route of administration, using a variety of approach known by those skilled in the art (e.g., by inhalation), and distribute to both the local target cells and tissues of the lung, as well as in peripheral non-lung cells and tissues (e.g., cells of the liver, spleen, kidneys, heart, skeletal muscle, lymph nodes, brain, cerebrospinal fluid, and plasma). As a result, both the local cells of the lung and the peripheral non-lung cells can serve as biological reservoirs or depots capable of producing and/or secreting a translation product encoded by one or more polynucleotides. Accordingly, the present invention is not limited to the treatment of lung diseases or conditions, but rather can be used as a non-invasive means of facilitating the delivery of polynucleotides, or the production of peptides or proteins encoded thereby, in peripheral organs, tissues and cells (e.g., hepatocytes) which would otherwise be achieved only by systemic administration. Exemplary peripheral non-lung cells include, but are not limited to, hepatocytes, epithelial cells, hematopoietic cells, epithelial cells, endothelial cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

Following administration of the composition to the subject, the peptide or protein product encoded by the mRNA (e.g., a functional protein or enzyme) is detectable in the peripheral target tissues for at least about one to seven days or longer following administration of the compound to the subject. The amount of peptide or protein product necessary to achieve a therapeutic effect will vary depending on the condition being treated, the peptide or protein encoded, and the condition of the patient. For example, the peptide or protein product may be detectable in the peripheral target tissues at a concentration (e.g., a therapeutic concentration) of at least 0.025-1.5 μg/ml (e.g., at least 0.050 μg/ml, at least 0.075 μg/ml, at least 0.1 μg/ml, at least 0.2 μg/ml, at least 0.3 μg/ml, at least 0.4 μg/ml, at least 0.5 μg/ml, at least 0.6 μg/ml, at least 0.7 μg/ml, at least 0.8 μg/ml, at least 0.9 μg/ml, at least 1.0 μg/ml, at least 1.1 μg/ml, at least 1.2 μg/ml, at least 1.3 μg/ml, at least 1.4 μg/ml, or at least 1.5 μg/ml), for at least about 1, 2, 3, 4, 5, 6, 7, 8, 9, 10, 11,12, 13, 14, 15, 16, 17, 18, 19, 20, 21, 22, 23, 24, 25, 26, 27, 28, 29, 30, 35, 40, 45 days or longer following administration of the compound to the subject.

It has been demonstrated that nucleic acids can be delivered to the lungs by intratracheal administration of a liquid suspension of the compound and inhalation of an aerosol mist produced by a liquid nebulizer or the use of a dry powder apparatus such as that described in U.S. Pat. No. 5,780,014, incorporated herein by reference.

In certain embodiments, the compounds of the invention may be formulated such that they may be aerosolized or otherwise delivered as a particulate liquid or solid prior to or upon administration to the subject. Such compounds may be administered with the assistance of one or more suitable devices for administering such solid or liquid particulate compositions (such as, e.g., an aerosolized aqueous solution or suspension) to generate particles that are easily respirable or inhalable by the subject. In some embodiments, such devices (e.g., a metered dose inhaler, jet-nebulizer, ultrasonic nebulizer, dry-powder-inhalers, propellant-based inhaler or an insufflator) facilitate the administration of a predetermined mass, volume or dose of the compositions (e.g., about 0.5 mg/kg of mRNA per dose) to the subject. For example, in certain embodiments, the compounds of the invention are administered to a subject using a metered dose inhaler containing a suspension or solution comprising the compound and a suitable propellant. In certain embodiments, the compounds of the invention may be formulated as a particulate powder (e.g., respirable dry particles) intended for inhalation. In certain embodiments, compositions of the invention formulated as respirable particles are appropriately sized such that they may be respirable by the subject or delivered using a suitable device (e.g., a mean D50 or D90 particle size less than about 500 μm, 400 μm, 300 μm, 250 μm, 200 μm, 150 μm, 100 μm, 75 μm, 50 μm, 25 μm, 20 μm, 15 μm, 12.5 μm, 10 μm, 5 μm, 2.5 μm or smaller). In yet other embodiments, the compounds of the invention are formulated to include one or more pulmonary surfactants (e.g., lamellar bodies). In some embodiments, the compounds of the invention are administered to a subject such that a concentration of at least 0.05 mg/kg, at least 0.1 mg/kg, at least 0.5 mg/kg, at least 1.0 mg/kg, at least 2.0 mg/kg, at least 3.0 mg/kg, at least 4.0 mg/kg, at least 5.0 mg/kg, at least 6.0 mg/kg, at least 7.0 mg/kg, at least 8.0 mg/kg, at least 9.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight is administered in a single dose. In some embodiments, the compounds of the invention are administered to a subject such that a total amount of at least 0.1 mg, at least 0.5 mg, at least 1.0 mg, at least 2.0 mg, at least 3.0 mg, at least 4.0 mg, at least 5.0 mg, at least 6.0 mg, at least 7.0 mg, at least 8.0 mg, at least 9.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg or at least 100 mg mRNA is administered in one or more doses.

### EXAMPLES

While certain compounds, compositions and methods of the present invention have been described with specificity in accordance with certain embodiments, the following examples serve only to illustrate the compounds of the invention and are not intended to limit the same.

### List of Abbreviations

ACN: Acetonitrile
Boc: tert-Butyloxycarbonyl
DIPEA: N,N-Diisopropylethylamine
DCM: Dichloromethane
DMAP: 4-Dimethylaminopyridine
EDC·HCl: 1-ethyl-3-(3-dimethylaminopropyl)carbodiimide hydrochloride
EtOAc: Ethyl acetate
THF: Tetrahydrofuran
IPA: isopropyl alcohol
LC-MS: Liquid chromatography-mass spectrometry
MeOH: Methanol
MS: Mass spectrometry
NaH: Sodium hydride
NaHCO₃: Sodium bicarbonate
Na₂SO₄: Sodium Sulfate
NH₄Cl: Ammonium chloride
NMR: Nuclear magnetic resonance spectroscopy
TFA: Trifluoroacetic Acid
TLC: Thin Layer Chromatography
TLC/ELSD: Thin Layer Chromatography/Evaporative Light Scattering Detector
Pd/C: Palladium on Carbon
NaOH: Sodium hydroxide
RT: Room temperature
SM: Starting material
SiO₂: Silicon dioxide
TBS: tert-butyldimethylsilyl
TBDMS: tert-butyldimethylsilyl
TLC: Thin Layer Chromatography Example 1: Synthesis of Compounds XXIV, XI, XXI, XXVII, XXVI, XXXV, XXX, XXXIII, XXVIII, XXXII, XXXIV, XXV, XXXVIII, XXXVI, XXXI and XXIX

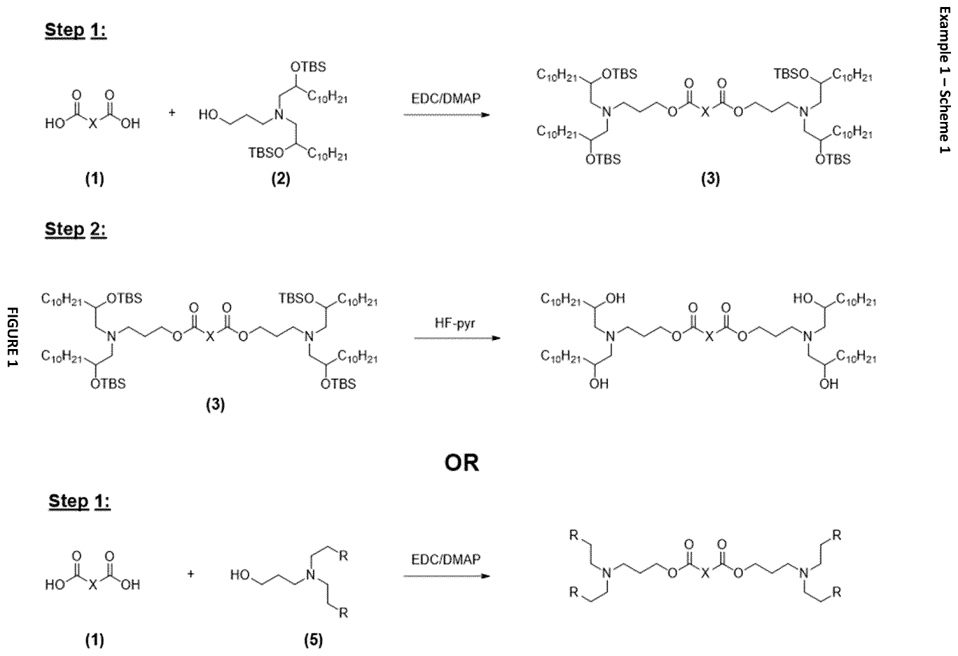

For example, the compounds of the invention may be prepared according to Scheme 1 (as depicted in FIG. 1).

### Synthesis of Compound XXIV

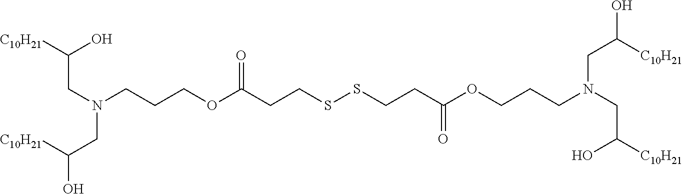

As depicted in Scheme 1, (wherein x is

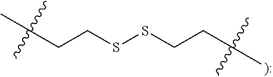 [SMILES: *CCSSCC*]

Step 1—To a 20 mL scintillation vial was added di-carboxylic acid (1) (71.1 mg, 1.0 equiv), alcohol intermediate (2) (500 mg, 2.2 equiv), DMAP (41.3 mg, 1.0 equiv), DIPEA (0.12 mL, 2.0 equiv), and anhydrous CH₂Cl₂ (7 mL). To this stirring solution at room temperature was added EDC (162 mg, 2.5 equiv) in one batch. The reaction was stirred at room temperature for 16 h and was monitored by TLC (10% EtOAc in hexanes). After significant consumption of (2) as determined by TLC, the reaction mixture was partitioned between layers of EtOAc and saturated NaHCO₃ aqueous solution. The separated aqueous layer was extracted with EtOAc (2×) before the combined organic layer was washed with brine and dried with sodium sulfate. Drying reagent was removed via filtration and the filtrate was concentrated under reduced pressure to provide crude product. Crude material was purified using medium pressure chromatography (Combiflash) using a gradient with 0-10% EtOAc in hexanes to isolate desired product (3) from the leftover starting material (2). The combined fractions containing (3) was concentrated to dryness to provide TBS ether intermediate (3) (426 mg, 83%) as viscous colourless oil. The identity of product was confirmed by MS.

### Results:

MS(ESI+) Calculated C₈₄H₁₇₆N₂O₈S₂Si₄, [M+H]⁺=1518.2, Observed=1518.9+760.0 [M/2].

As depicted in Scheme 1, (wherein x is

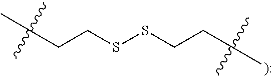 [SMILES: *CCSSCC*]

Step 2—To a plastic 20 mL scintillation vial was added TBS ether (3) (426 mg, 1.0 equiv) and anhydrous THF (4 mL). The resulting solution was stirred and cooled to 0° C. using ice bath before a 70% HF-pyridine solution was added dropwise (1.44 mL, 197 equiv of HF) and stirred at the same temperature for 5 minutes before allowed to warm up slowly to room temperature and stirred at room temperature over 16 h. After completion of reaction as monitored by MS, the reaction mixture was cooled to 0° C. and quenched with batch-wise addition of solid NaHCO₃. After gas formation has minimized, the resulting mixture was diluted with EtOAc and neutralized with aqueous NaHCO₃ until pH=7-8. The neutralized aqueous layer was extracted with EtOAc (2×) and the combined organic layers were washed with brine and dried with sodium sulfate. Drying reagent was removed via filtration and the filtrate was concentrated under reduced pressure to provide crude product. Crude material was purified using 0-60% EtOAc in hexanes gradient to obtain Compound XXIV (246 mg, 83%) as colourless viscous oil.

### Results:

MS(ESI+) Calculated C₆₀H₁₂₀N₂O₈S2, [M+H]+=1061.8, Observed=1061.7 and 531.4 [M/2].

### Synthesis of Compound XI

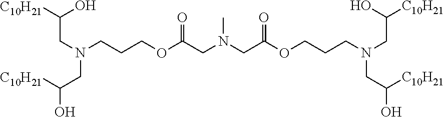

As depicted in Scheme 1, (wherein x is

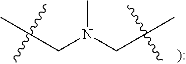 [SMILES: *CN(C)C*]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XI (97 mg) as colorless viscous oil.

### Results:

MS(ESI+) Calculated C₅₉H₁₁₉N₃O₈, [M+H]⁺=998.9, Observed=998.8 and 500.0 [M/2].

### Synthesis of Compound XXI

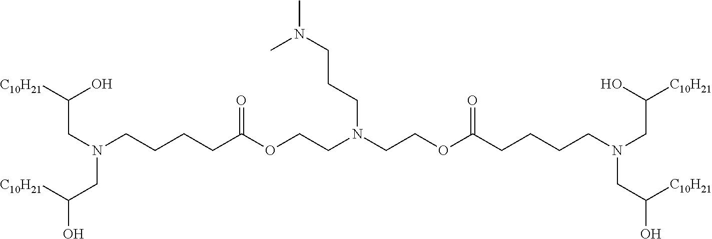

As depicted in Scheme 1, (wherein x is

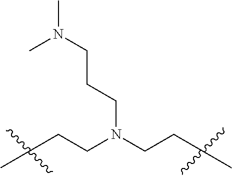 [SMILES: *CCN(CC*)CCCN(C)C]

and the esters bound to x are reversed): The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXI (135 mg) as colorless viscous oil.

### Results:

MS(ESI+) Calculated C₆₇H₁₃₆N₄O₈, [M+H]⁺=1126.0, Observed=1125.9 and 563.5 [M/2].

### Synthesis of Compound XXVII

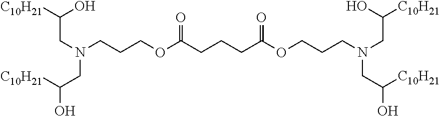

As depicted in Scheme 1, (wherein x is

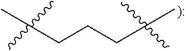 [SMILES: *CCC*]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXVII (272 mg) as colorless viscous oil.

### Results:

MS(ESI+) Calculated C₅₉H₁₁₈N₂O₈, [M+H]⁺=983.9, Observed=984.4 and 492.8 [M/2].

### Synthesis of Compound XXVI

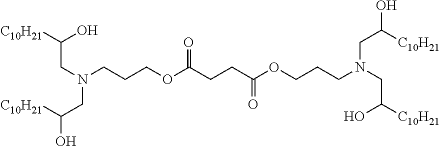

As depicted in Scheme 1, (wherein x is

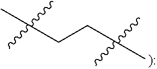 [SMILES: *CC*]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXVI (174 mg) as colorless viscous oil.

### Results:

MS(ESI+) Calculated C₅₈H₁₁₆N₂O₈, [M+H]⁺=969.9, Observed=969.2 and 485.1 [M/2].

### Synthesis of Compound XXXV

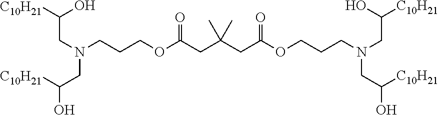

As depicted in Scheme 1, (wherein x is

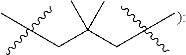 [SMILES: *CC(C)(C)C*]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXXV (33 mg) as colorless viscous oil.

### Results:

MS(ESI+) Calculated C₆₁H₁₂₂N₂O₈, [M+H]⁺=1011.9, Observed=1012.2 and 506.1 [M/2].

### Synthesis of Compound XXX

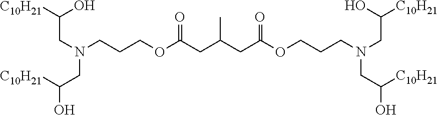

As depicted in Scheme 1, (wherein x is

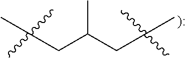 [SMILES: *CC(C)C*]

The procedure for Compound XXIV was followed using di-carboxylic acid (1) (1.0 equiv and common alcohol intermediate (2) (2.2 equiv) to obtain the desired intermediate (3). After confirmation by MS, TBS ether (3) was treated with the 70% HF-pyridine solution to provide Compound XXX (81 mg) as colorless viscous oil.

### Results:

MS(ESI+) Calculated CH₆₀H₁₂₀N₂O₈, [M+H]⁺=997.9, Observed=997.2 and 499.3 [M/2].

### Synthesis of Compound XXXIII

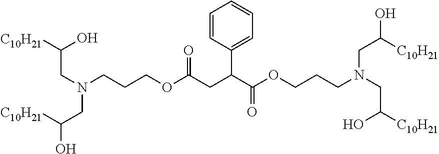

As depicted in Scheme 1, (wherein x is

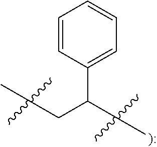 [SMILES: *CC(*)c1ccccc1]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXXIII (113 mg) as colorless viscous oil.

### Results:

MS(ESI+) Calculated C₆₄H₁₂₀N₂O₈, [M+H]⁺=1045.9, Observed=1045.2 and 523.2 [M/2].

### Synthesis of Compound XXVIII

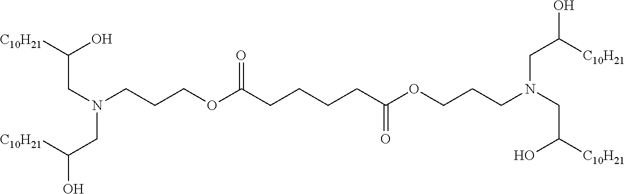

As depicted in Scheme 1, (wherein x is

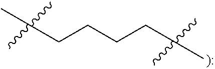 [SMILES: *CCCC*]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXVIII (120 mg) as colorless viscous oil.

### Results:

MS(ESI+) Calculated CH₆₀H₁₂₀N₂O₈, [M+H]⁺=997.9, Observed=998.2 and 499.1 [M/2].

### Synthesis of Compound XXXII

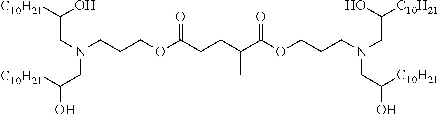

As depicted in Scheme 1, (wherein x is

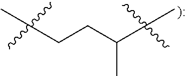 [SMILES: *CCC(*)C]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXXII (150 mg) as colorless viscous oil.

### Results:

MS(ESI+) Calculated C₆₀H₁₂₀N₂O₈, [M+H]⁺=997.9, Observed=998.2 and 499.1 [M/2].

### Synthesis of Compound XXXIV

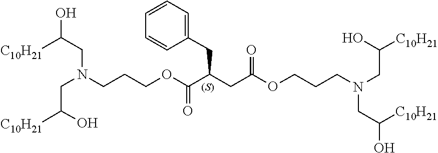

As depicted in Scheme 1, (wherein x is

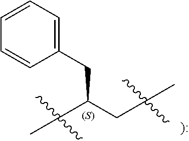

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXXIV (101 mg) as colorless viscous oil.

### Results:

MS(ESI+) Calculated C₆₅H₁₂₂N₂O₈, [M+H]⁺=1059.9, Observed=1059.2 and 530.3 [M/2].

### Synthesis of Compound XXV

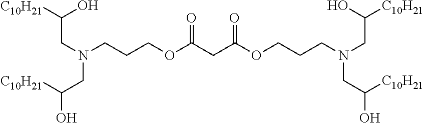

As depicted in Scheme 1, (wherein x is

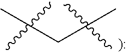 [SMILES: *C*]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXV (31 mg) as colorless viscous oil.

### Results:

MS(ESI+) Calculated C₅₇H₁₁₄N₂O₈, [M+H]⁺=955.9, Observed=955.2 and 478.1 [M/2].

### Synthesis of Compound XXXVIII

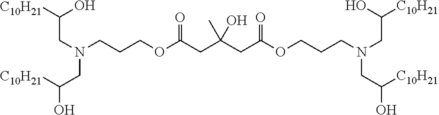

As depicted in Scheme 1, (wherein x is

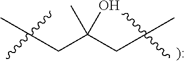 [SMILES: *CC(C)(O)C*]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXXVIII (55 mg) as colorless viscous oil.

### Results:

MS(ESI+) Calculated C₆₀H₁₂₀N₂O₉, [M+H]⁺=1013.9, Observed=1013.1 and 507.2 [M/2].

### Synthesis of Compound XXXVI

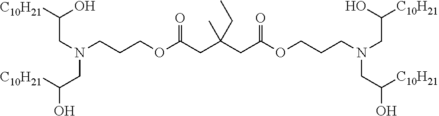

As depicted in Scheme 1, (wherein x is

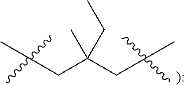 [SMILES: *CC(C)(C*)CC]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXXVI (56 mg) as colorless viscous oil.

### Results:

MS(ESI+) Calculated C₆₂H₁₂₄N₂O₈, [M+H]⁺=1025.9, Observed=1025.3 and 513.3 [M/2].

### Synthesis of Compound XXXI

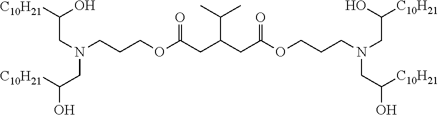

As depicted in Scheme 1, (wherein x is

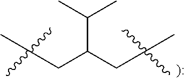 [SMILES: *CC(C*)C(C)C]

The procedure for Compound XXIV was followed using di-carboxylic acid (1) (1.0 equiv) and common alcohol intermediate (2) (2.2 equiv) to obtain the desired intermediate (3). After confirmation by MS, TBS ether (3) was treated with the 70% HF-pyridine solution to provide Compound XXXI (113 mg) as colorless viscous oil.

### Results:

MS(ESI+) Calculated C₆₂H₁₂₄N₂O₈, [M+H]⁺=1025.9, Observed=1026.8 and 514.0 [M/2].

### Synthesis of Compound XXIX

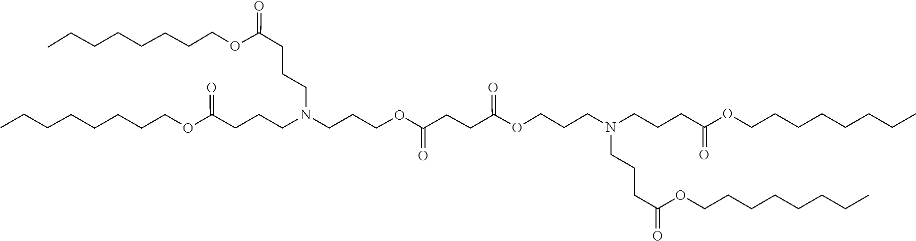 [SMILES: CCCCCCCCOC(=O)CCCN(CCCOC(=O)CCC(=O)OCCCN(CCCC(=O)OCCCCCCCC)CCCC(=O)OCCCCCCCC)CCCC(=O)OCCCCCCCC]

As depicted in Scheme 1, (wherein x is

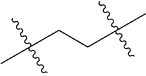 [SMILES: *CC*]

### and R is

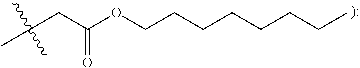 [SMILES: *CC(=O)OCCCCCCCC]

To a 20 mL scintillation vial was added di-carboxylic acid (1) (20 mg, 1.0 equiv), synthesized alcohol intermediate (5) (222 mg, 2.78 equiv), DMAP (27.4 mg, 1.3 equiv), DIPEA (0.08 mL, 2.65 equiv), and anhydrous CH₂Cl₂ (5 mL). To this stirring solution at room temperature was added EDC (107 mg, 3.3 equiv) in one batch. The reaction was stirred at room temperature for 16 h and was monitored by TLC (10% EtOAc in hexanes). After significant consumption of (5) as determined by TLC, the reaction mixture was partitioned between layers of EtOAc and saturated NaHCO₃ aqueous solution. The separated aqueous layer was extracted with EtOAc (2×) before the combined organic layer was washed with brine and dried with sodium sulfate. Drying reagent was removed via filtration and the filtrate was concentrated under reduced pressure to provide crude product. Crude material was purified using Combiflash using 50% EtOAc in hexanes to isolate desired product Compound XXIX. The combined fractions containing Compound XXIX was concentrated to dryness to provide lipid product Compound XXIX (90 mg, 53%) as viscous colourless oil.

### Results:

MS(ESI+) Calculated C₈₄H₁₇₆N₂O₃S₂Si₄, [M+H]⁺=1025.8, Observed=1025.2 and 513.1 [M/2].

### Example 2: Synthesis of Compounds V and XVI

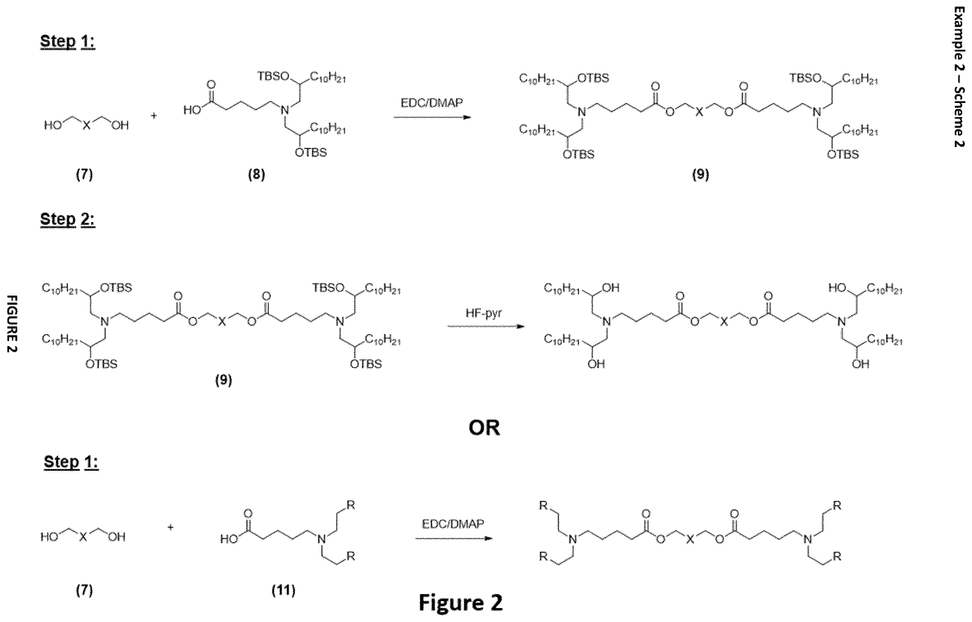

For example, the compounds of the invention may be prepared according to Scheme 2 (as depicted in FIG. 2).

### Synthesis of Compound V

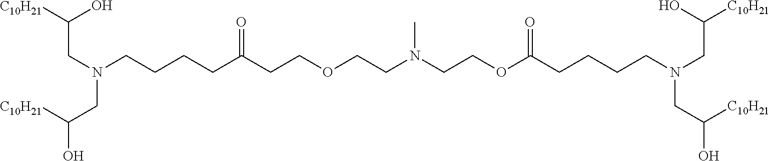

As depicted in Scheme 2, (wherein x is

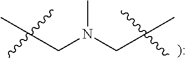 [SMILES: *CN(C)C*]

Step 1—To a 20 mL scintillation vial was added amino diol (7) (37.5 mg, 0.45 equiv), acid intermediate (8) (500 mg, 1.0 equiv), DMAP (85.5 mg, 1.0 equiv), DIPEA (0.73 mL, 6.0 equiv), and anhydrous CH₂Cl₂ (7 mL). To this stirring solution at room temperature was added EDC (268 mg, 2.0 equiv) in one batch. The reaction was stirred at room temperature for 16 h and was monitored by TLC (10% EtOAc in hexanes). After significant consumption of (8) as determined by TLC, the reaction mixture was partitioned between layers of EtOAc and saturated NaHCO₃ aqueous solution. The separated aqueous layer was extracted with EtOAc (2×) before the combined organic layer was washed with brine and dried with sodium sulfate. Drying reagent was removed via filtration and the filtrate was concentrated under reduced pressure to provide crude product. Crude material was purified using Combiflash using 0-10% EtOAc in hexanes to isolate desired product (9) from the leftover starting material (8). The combined fractions containing product was concentrated to dryness to provide TBS ether intermediate (9) (318 mg, 67%) as viscous colourless oil. The identity of product was confirmed by MS.

### Results:

MS(ESI+) Calculated C₃₇H₁₈₃N₃O₈Si₄, [M+H]⁺=1511.3, Observed=1512.2.

As depicted in Scheme 2, (wherein x is

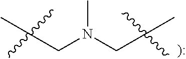 [SMILES: *CN(C)C*]

Step 2—To a plastic 20 mL scintillation vial was added TBS ether (9) (318 mg, 1.0 equiv) and anhydrous THF (3 mL). The resulting solution was stirred and cooled to 0° C. using ice bath before a 70% HF-pyridine solution was added dropwise (1.08 mL, 197 equiv of HF) and stirred at the same temperature for 5 minutes before allowed to warm up slowly to room temperature and stirred at room temperature over 16 h. After completion of reaction as monitored by MS, the reaction mixture was cooled to 0° C. and quenched with batch-wise addition of solid NaHCO₃. After gas formation has minimized, the resulting mixture was diluted with EtOAc and neutralized with aqueous NaHCO₃ until pH=7-8. The neutralized aqueous layer was extracted with EtOAc (2×) and the combined organic layers were washed with brine and dried with sodium sulfate. Drying reagent was removed via filtration and the filtrate was concentrated under reduced pressure to provide crude product. Crude material was purified using 0-20% MeOH in CH₂Cl₂ gradient to obtain Compound V (181 mg, 82%) as colourless viscous oil.

### Results:

MS(ESI+) Calculated C₆₃H₁₂₇N₃O₈, [M+H]⁺=1055.0, Observed=1054.8.

### Synthesis of Compound XVI

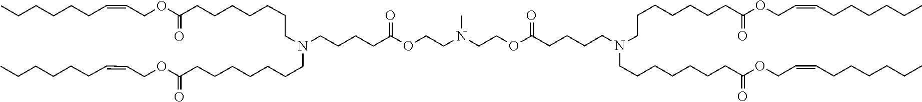 [SMILES: CCCCCC/C=C\COC(=O)CCCCCCCN(CCCCCCCC(=O)OC/C=C\CCCCCC)CCCCC(=O)OCCN(C)CCOC(=O)CCCCN(CCCCCCCC(=O)OC/C=C\CCCCCC)CCCCCCCC(=O)OC/C=C\CCCCCC]

As depicted in Scheme 2, (wherein x is

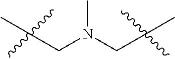 [SMILES: *CN(C)C*]

### and R is

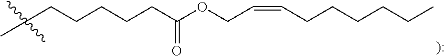 [SMILES: *CCCCCC(=O)OC/C=C\CCCCCC]

To a 20 mL scintillation vial was added amino diol (7) (21.0 mg, 1.0 equiv), acid intermediate (11) (270 mg, 2.0 equiv), DMAP (21.6 mg, 1.0 equiv), DIPEA (0.13 mL, 4.2 equiv), and anhydrous CH₂Cl₂ (3.5 mL). To this stirring solution at room temperature was added EDC (84.7 mg, 2.5 equiv) in one batch. The reaction was stirred at room temperature for 16 h and was monitored by MS. After significant consumption of (11) as determined by MS, the reaction mixture was partitioned between layers of EtOAc and saturated NaHCO₃ aqueous solution. The separated aqueous layer was extracted with EtOAc (2×) before the combined organic layer was washed with brine and dried with sodium sulfate. Drying reagent was removed via filtration and the filtrate was concentrated under reduced pressure to provide crude product. Crude material was purified using Combiflash using 10% MeOH in CH₂Cl₂ to isolate desired product Compound XVI. The combined fractions containing product was concentrated to dryness to provide lipid product Compound XVI (65 mg, 27%) as viscous colourless oil.

### Results:

MS(ESI+) Calculated C₃₃H₁₅₁N₃O₁₂, [M+H]⁺=1383.1, Observed=1383.2.

### Example 3: Synthesis of Compound XLI

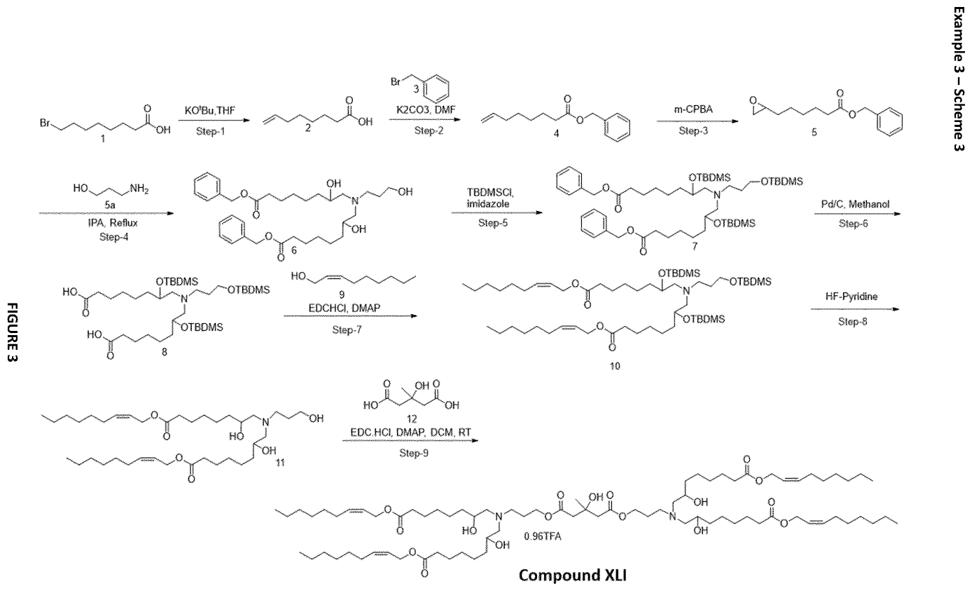

For example, the compounds of the invention may be prepared according to Scheme 3 (as depicted in FIG. 3).

### Synthesis of Intermediate [2]

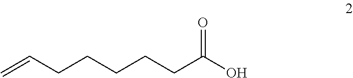

As depicted in Scheme 3: To the stirred solution of 8-bromooctanoic acid [1](15.0 g, 67.2 mmol) in tetrahydrofuran (0.5 L, 6.14 mol) was added potassium 2-methylpropan-2-olate (33.9 g, 303 mmol). The reaction mixture was stirred at 90° C. for 16 h. TLC shows SM consumed and formed new spot. The reaction mixture was dilute with cold water (500 mL) and acidified by using 2N aq. HCl up to 2-3 pH then extract with EtOAc (2×500 mL). The organic layer was dried over anhy. Na₂SO₄, filtered and evaporated to give oct-7-enoic acid [2] (10.0 g, crude) as pale yellow oil. Crude used as such for next step.

### Results:

1H-NMR (400 MHz, CDCl3)-10.5-11.00 (brs, 1H), 5.84-5.73 (m, 1H), 5.00-4.90 (m, 2H), 2.31-2.28 (t, J=7.6 Hz, 2H), 2.06-2.01 (q, J=7.6 Hz, 2H), 1.65-1.58 (m, 2H), 1.46-1.36 (m, 4H) ppm.

### Synthesis of Intermediate [4]

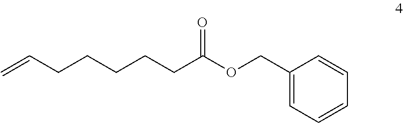 [SMILES: C=CCCCCCC(=O)OCc1ccccc1]

As depicted in Scheme 3: To the stirred solution of oct-7-enoic acid [2](10 g, 70.3 mmol) in dimethylformamide (200 mL) was added dipotassium carbonate (29.2 g, 211 mmol) followed by the addition of (bromomethyl)benzene [3](10 mL, 84.4 mmol) at RT. The reaction allowed to stir at RT for 16 h. Progress of reaction was monitored by TLC/ELSD. The reaction mixture was diluted with cold water (200 mL) and extracted with diethyl ether (2×500 mL). Organic layer was wash with saturated aq. NaHCO3 (500 mL) and brine (500 mL), dried over anhy. Na₂SO₄, filtered and concentrated. Crude obtained was purify over silica gel flash column chromatography (0-10% ethyl acetate in heptane) to give benzyl oct-7-enoate [4](6.5 g, 39.7% Yield) as pale yellow oil.

### Results:

1H-NMR (400 MHz, CDCl3)-7.39-7.30 (m, 5H), 5.84-5.74 (m, 1H), 5.11 (s, 2H), 5.01-4.92 (m, 2H), 2.38-2.34 (t, J=7.6 Hz, 2H), 2.06-2.01 (q, J=7.6 Hz, 2H), 1.69-1.61 (m, 2H), 1.44-1.27 (m, 4H) ppm.

### Synthesis of Intermediate [5]

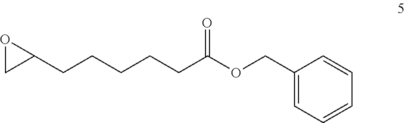 [SMILES: O=C(CCCCCC1CO1)OCc1ccccc1]

As depicted in Scheme 3: To a stirred solution of benzyl oct-7-enoate [4](6.5 g, 28 mmol) in dichloromethane (50 mL) was added 3-chlorobenzene-1-carboperoxoic acid (14.5 g, 83.9 mmol) to the reaction mass at 0° C., under nitrogen atmosphere. Reaction mixture allowed to stir at RT for 16 h. After 16 h, reaction progress was monitored by TLC. The reaction mass was diluted with DCM (50 mL), washed with saturated aq. solution of NaHCO₃ (100 mL) and brine (100.0 mL). Organic layer dried over anhy. Na₂SO₄, filtered and evaporated under reduce pressure. Crude was purified over silica gel flash column chromatography (0-10% ethyl acetate in heptane gradient) to give benzyl 6-(oxiran-2-yl)hexanoate [5](5.5 g, 79% Yield) as yellow oil.

### Results:

1H-NMR (400 MHz, CDCl3)-7.37-7.32 (m, 5H), 5.11 (s, 2H), 2.90-2.87 (m, 1H), 2.75-2.73 (m, 1H), 2.46-2.44 (m, 1H), 2.39-2.35 (t, J=7.6 Hz, 2H), 1.70-1.63 (m, 2H), 1.56-1.43 (m, 4H), 1.39-1.26 (m, 2H) ppm.

### Synthesis of Intermediate [6]

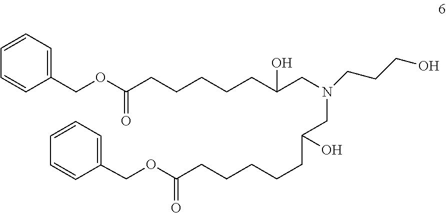 [SMILES: O=C(CCCCCC(O)CN(CCCO)CC(O)CCCCCC(=O)OCc1ccccc1)OCc1ccccc1]

As depicted in Scheme 3: To a stirred solution of benzyl 6-(oxiran-2-yl)hexanoate [5](7.73 g, 31.1 mmol) in isopropanol (0.1 L), added 3-aminopropan-1-ol [5a](1.11 g, 14.8 mmol) at RT under inert atmosphere. Resultant reaction mixture was stirred at 95° C. for 20 h. Progress of reaction was monitored by TLC. Reaction mass evaporated under reduced pressure to gives crude, which was purify over silica gel flash column chromatography by using 3-5% MeOH in DCM gradient as eluent to give benzyl 8-{[8-(benzyloxy)-2-hydroxy-8-oxooctyl](3-hydroxypropyl)amino}-7-hydroxyoctanoate [6](3.2 g, 37.7% Yield) as colourless oil.

### Results:

ELSD analysis: Purity 99.02%, Calculated C33H49NO7=571.35, Observed=572.30 (m/z, M+H+).

### Synthesis of Intermediate [7]

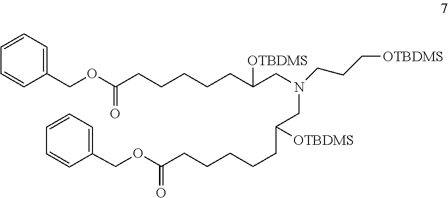 [SMILES: CC(C)(C)[Si](C)(C)OCCCN(CC(CCCCCC(=O)OCc1ccccc1)O[Si](C)(C)C(C)(C)C)CC(CCCCCC(=O)OCc1ccccc1)O[Si](C)(C)C(C)(C)C]

As depicted in Scheme 3: To a stirred solution of benzyl 8-{[8-(benzyloxy)-2-hydroxy-8-oxooctyl](3-hydroxypropyl)amino}-7-hydroxyoctanoate [6](3.2 g, 5.6 mmol) in dichloromethane (0.1 L), was added tert-butyl(chloro)dimethylsilane (6.75 g, 44.8 mmol) and 1H-imidazole (5.33 g, 78.4 mmol) at RT, under inert atmosphere. Resultant reaction mass allowed to stir at RT for 16 h. Progress of reaction was monitor by TLC. Reaction mass was filtered through sintered funnel. Filtrate was evaporated under reduced pressure to gives crude, which was purify over silica gel flash column chromatography (0-20% ethylacetate in heptane) to give benzyl 8-{5-[6-(benzyloxy)-6-oxohexyl]-2,2,3,3,12,12,13,13-octamethyl-4,11-dioxa-7-aza-3,12-disilatetradecan-7-yl}-7-[(tert-butyldimethylsilyl)oxy]octanoate [7](3.5 g, 68.4% Yield) as colorless liquid.

### Results:

ELSD analysis: Purity 97.95%, Calculated C51H91NO7Si3=913.61, Observed=914.50 (m/z, M+H+).

### Synthesis of Intermediate [8]

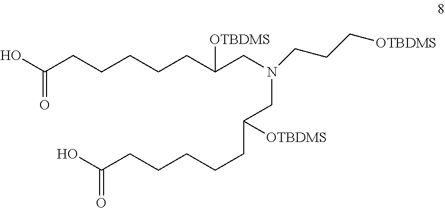 [SMILES: CC(C)(C)[Si](C)(C)OCCCN(CC(CCCCCC(=O)O)O[Si](C)(C)C(C)(C)C)CC(CCCCCC(=O)O)O[Si](C)(C)C(C)(C)C]

As depicted in Scheme 3: To a stirred solution of benzyl 8-{5-[6-(benzyloxy)-6-oxohexyl]-2,2,3,3,12,12,13,13-octamethyl-4,11-dioxa-7-aza-3,12-disilatetradecan-7-yl}-7-[(tert-butyldimethylsilyl)oxy]octanoate [7](4.3 g, 4.7 mmol), in methanol (15 mL), tetrahydrofuran (15 mL), added palladium on carbon (10% w/w, with 50% moisture) (1.5 g, 14.1 mmol) portion wise under nitrogen atmosphere. Resultant reaction mass was degassed and purged with Hydrogen at RT, then allowed to stir at hydrogen balloon pressure for 16 h. After, completion of reaction, reaction mixture was filtered through celite, washed the celite bed two times with methanol. Methanol was evaporated to dryness to get 7-[(tert-butyldimethylsilyl)oxy]-8-[5-(5-carboxypentyl)-2,2,3,3,12,12,13,13-octamethyl-4,11-dioxa-7-aza-3,12-disilatetradecan-7-yl]octanoic acid [8](3.4 g, 98% Yield) as colourless liquid.

### Results:

ELSD analysis: Purity 99.33%, Calculated C37H79NO7Si3=733.52, Observed=734.50 (m/z, M+H+).

### Synthesis of Intermediate [10]

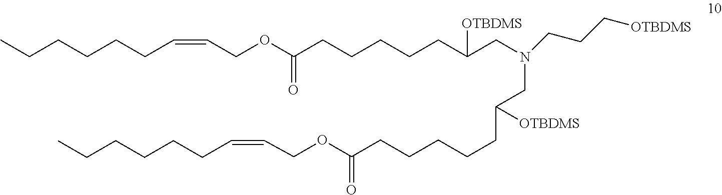 [SMILES: CCCCCC/C=C\COC(=O)CCCCCC(CN(CCCO[Si](C)(C)C(C)(C)C)CC(CCCCCC(=O)OC/C=C\CCCCCC)O[Si](C)(C)C(C)(C)C)O[Si](C)(C)C(C)(C)C]

As depicted in Scheme 3: To a stirred solution of 7-[(tert-butyldimethylsilyl)oxy]-8-[5-(5-carboxypentyl)-2,2,3,3,12,12,13,13-octamethyl-4,11-dioxa-7-aza-3,12-disilatetradecan-7-yl]octanoic acid [8](3.5 g, 4.77 mmol) in dichloromethane (70 mL), added {3-[cyano(ethyl)amino]propyl}dimethylazanium chloride (2.74 g, 14.3 mmol) and DMAP (587 mg, 4.77 mmol) at RT under inert atmosphere, then after 15 min was added (2Z)-non-2-en-1-ol [9](1.69 g, 11.9 mmol) at RT under inert atmosphere. To the resultant reaction mixture was stirred at RT for 16 h. Progress of reaction was monitored by TLC. Reaction mass quench with water (100 mL) and extracted with DCM (3×100 mL). Combined organic layer was wash with brine and dried over sodium sulphate, filtered and evaporated under reduced pressure. Crude was purify over silica gel flash column chromatography (0-10% Ethyl acetate in heptane) to give (2Z)-non-2-en-1-yl 7-[(tert-butyldimethylsilyl)oxy]-8-(2,2,3,3,12,12,13,13-octamethyl-5-{6-[(2Z)-non-2-en-1-yloxy]-6-oxohexyl}-4,11-dioxa-7-aza-3,12-disilatetradecan-7-yl)octanoate [10](3.7 g, 79% Yield) as colourless liquid.

### Results:

ELSD analysis: Purity 96.75%, Calculated C₅₅H₁₁₁NO₇Si₃=981.77, Observed=982.60 (m/z, M+H+).

### Synthesis of Intermediate [11]

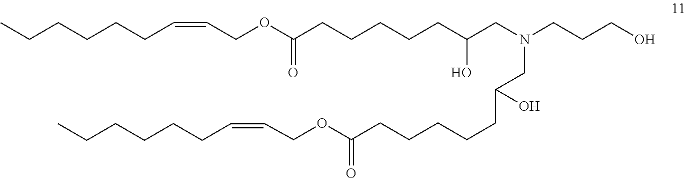 [SMILES: CCCCCC/C=C\COC(=O)CCCCCC(O)CN(CCCO)CC(O)CCCCCC(=O)OC/C=C\CCCCCC]

As depicted in Scheme 3: To a stirred solution of (2Z)-non-2-en-1-yl 7-[(tert-butyldimethylsilyl)oxy]-8-(2,2,3,3,12,12,13,13-octamethyl-5-{6-[(2Z)-non-2-en-1-yloxy]-6-oxohexyl}-4,11-dioxa-7-aza-3,12-disilatetradecan-7-yl)octanoate [10](3.6 g, 3.66 mmol) in tetrahydrofuran (30 mL), added pyridine hydro fluoride complex (1.45 g, 14.7 mmol) at 0° C. then allowed to stir for 16 h. The progress of reaction was monitored by TLC. After completion of reaction, reaction mixture was quenched with saturated aq. solution of sodium bicarbonate solution up to pH 8 and extracted with ethyl acetate (3×100 mL). The organic layer was dried over anhy. sodium sulphate, filtered and concentrated under reduced pressure. Crude was purified over silica gel flash column chromatography (0-5% MeOH in DCM) to give (2Z)-non-2-en-1-yl 7-hydroxy-8-({2-hydroxy-8-[(2Z)-non-2-en-1-yloxy]-8-oxooctyl}(3-hydroxypropyl)amino)octanoate [11](1.8 g, 76% Yield) as pale yellow liquid.

### Results:

ELSD analysis: Purity 98.33%, Calculated C₃₇H₆₉NO₇=639.51, Observed=640.45 (m/z, M+H+).

### Synthesis of Compound XLI

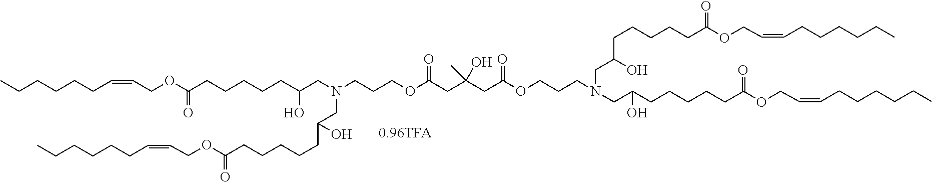 [SMILES: CCCCCC/C=C\COC(=O)CCCCCC(O)CN(CCCOC(=O)CC(C)(O)CC(=O)OCCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC]

As depicted in Scheme 3: A stirred solution of (2Z)-non-2-en-1-yl 7-hydroxy-8-({2-hydroxy-8-[(2Z)-non-2-en-1-yloxy]-8-oxooctyl}(3-hydroxypropyl)amino)octanoate [11](829 mg, 1.3 mmol) and 3-hydroxy-3-methylpentanedioic acid [12](0.1 g, 617 μmol) in dichloromethane (15 mL) was cooled to 0° C., added {3-[cyano(ethyl)amino]propyl}dimethylazanium chloride (355 mg, 1.85 mmol) and 4-(dimethylamino)pyridin-1-ium (228 mg, 1.85 mmol) successively at RT. Resultant reaction mixture was stir at RT for 48 h. The progress of reaction was monitored by ELSD/TLC (SM was consumed). Reaction mass was diluted with DCM (30 mL), washed with water (50 mL). Organic layer was dried over Na₂SO₄, filtered and concentrated under reduce pressure, and the crude was purified by prep HPLC (ACN/0.1% TFA in water) give the desired 1,5-bis({3-[bis({2-hydroxy-8-[(2Z)-non-2-en-1-yloxy]-8-oxooctyl})amino]propyl}) 3-hydroxy-3-methylpentanedioate.TFA salt Compound XLI (0.1 g, 11.5% Yield) as colourless liquid.

### Results:

1H-NMR (400 MHz, CDCl3)-5.67-5.60 (m, 4H), 5.34-5.48 (m, 4H), 4.62-4.60 (d, J=6.8 Hz, 8H), 4.26-4.22 (m, 4H), 4.12-4.00 (m, 4H), 3.49-3.38 (m, 4H), 3.22-3.05 (m, 8H), 2.77-2.70 (m, 2H), 2.61-2.54 (m, 4H), 2.33-2.29 (t, J=7.6 Hz, 8H), 2.20-2.10 (m, 4H), 2.10-2.06 (m, 8H), 1.65-1.58 (m, 8H), 1.58-1.42 (m, 12H), 1.41-1.27 (m, 45H) 0.89-0.86 (t, J=7.2 Hz, 12H) ppm.

ELSD analysis: Purity 99.95%, Calculated C₃₀H₁₄₄N₂O₁₇=1405.05, Observed=1405.85 (m/z, M+H+).

### Example 4: Synthesis of Compound LXXII

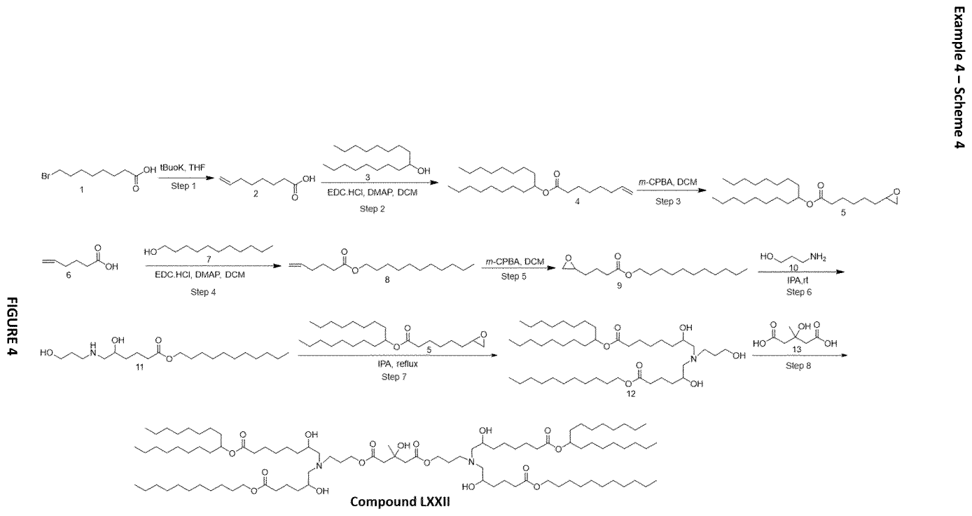

For example, the compounds of the invention may be prepared according to Scheme 4 (as depicted in FIG. 4).

### Synthesis of Intermediate [2]

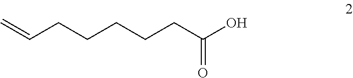

As depicted in Scheme 4: To the stirred solution of 8-bromooctanoic acid [1](15.0 g, 67.2 mmol) in tetrahydrofuran (0.5 L, 6.14 mol) was added potassium 2-methylpropan-2-olate (33.9 g, 303 mmol). The reaction mixture was stirred at 90° C. for 16 h. TLC shows SM consumed and formed new spot. The reaction mixture was dilute with cold water (500 mL) and acidified by using 2N aq. HCl up to 2-3 pH then extract with EtOAc (2×500 mL). The organic layer was dried over anhy. Na₂SO₄, filtered and evaporated to give oct-7-enoic acid (10.0 g, crude) as pale yellow oil. Crude used as such for next step.

### Results:

1H-NMR (400 MHz, CDCl3)-10.5-11.00 (brs, 1H), 5.84-5.73 (m, 1H), 5.00-4.90 (m, 2H), 2.31-2.28 (t, J=7.6 Hz, 2H), 2.06-2.01 (q, J=7.6 Hz, 2H), 1.65-1.58 (m, 2H), 1.46-1.36 (m, 4H) ppm.

### Synthesis of Intermediate [4]

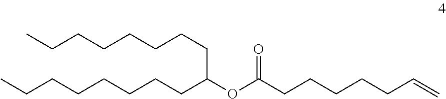 [SMILES: C=CCCCCCC(=O)OC(CCCCCCCC)CCCCCCCC]

As depicted in Scheme 4: To a stirred solution of oct-7-enoic acid [2](6.8 g, 47.8 mmol) in dichloromethane (100 mL, 469 mmol) added DMAP (5.89 g, 47.8 mmol) and ({[3-(dimethylamino)propyl]imino}methylidene)(ethyl)amine hydrochloride (18.3 g, 95.6 mmol) at room temperature. After this heptadecan-9-ol [3](13.5 g, 52.6 mmol) was added in reaction mixture and reaction mixture was stirred for 16 h at RT. The reaction was monitored by TLC, after completion the reaction, reaction mixture was diluted with DCM and washed with brine solution. The organic layers were combined, dried over sodium sulphate, concentrated under reduced pressure to get crude and crude used for column chromatography (0-5% ethyl acetate) to get desired product heptadecan-9-yl oct-7-enoate [4](10.5 g, 57.68%, Yield) as colourless liquid.

### Results:

1H NMR (400 MHz, CDCl3): δ 5.83-5.74 (m, 1H), 5.02-4.98 (m, 1H), 4.97-4.92 (m, 1H), 4.88-4.85 (m, 1H), 2.30-2.26 (t, J=7.6 Hz, 2H), 2.07-2.02 (q, J=6.8 Hz, 2H), 1.65-1.60 (m, 2H), 1.56-1.49 (m, 4H), 1.42-1.25 (m, 28H), 0.89-0.86 (t, J=6.8 Hz, 6H) ppm.

### Synthesis of Intermediate [5]

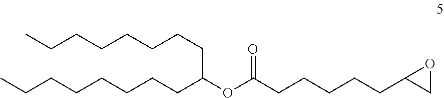 [SMILES: CCCCCCCCC(CCCCCCCC)OC(=O)CCCCCC1CO1]

As depicted in Scheme 4: To a stirred solution of heptadecan-9-yl oct-7-enoate [4](10.5 g, 52.5 mmol) in dichloromethane (200 mL), 3-chlorobenzene-1-carboperoxoic acid (10.5 g, 60.7 mmol) was added at 0° C. The resulting reaction mixture was stirred for 16 h at room temperature. The progress of reaction mass was monitored by ELSD/TLC (SM was consumed). The resulting reaction mixture was washed with cold aqueous sodium bicarbonate solution (500 mL). The resulting organic layer was dried over Na₂SO₄, and concentrated under reduce pressure, and the crude was purified by flash column chromatography (SiO₂: 0-5% ethyl acetate in hexane), to give the desired heptadecan-9-yl 6-(oxiran-2-yl)hexanoate [5](9.2 g, 84.08%, Yield) as a colourless liquid.

### Results:

1H-NMR (400 MHz, CDCl3)-4.86 (q, J=6.0 Hz, 1H), 2.92-2.87 (br, 1H), 2.75-2.73 (m, 1H), 2.46-2.44 (m, 1H), 2.29 (t, J=7.6 Hz, 2H), 1.66-1.62 (m, 2H), 1.55-1.44 (m, 7H), 1.41-1.36 (m, 2H), 1.25 (br, 25H), 0.89-0.85 (t, J=6.8 Hz, 6H).

ELSD analysis: Purity 99.93%, Calculated C₂₅H₄₈O₂=396.36, Observed=397.20 (m/z, M+H+) & 419.35 (m/z, M+Na+).

### Synthesis of Intermediate [8]

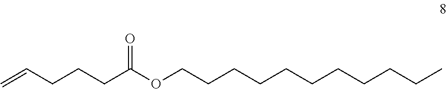 [SMILES: C=CCCCC(=O)OCCCCCCCCCCC]

As depicted in Scheme 4: To a solution of hex-5-enoic acid [6](10 g, 87.6 mmol), {3-[cyano(ethyl)amino]propyl}dimethylazanium chloride (25.2 g, 131 mmol), and DMAP (5.4 g, 43.8 mmol) in dichloromethane (150 mL) at room temperature. After this undecan-1-ol [7](13.6 g, 78.8 mmol) was added in reaction mixture, and reaction mixture was stirred at RT for 16 h. The reaction was monitored by TLC, after completion the reaction, reaction mixture was diluted with DCM and washed with brine solution. The organic layers were combined, dried over sodium sulphate, concentrated under reduced pressure. and the crude was purified by flash column chromatography (SiO₂: 0-5% ethyl acetate in hexane), to obtain the desired produced undecyl hex-5-enoate [8](19.8 g, 84.19%, Yield) as a colorless oil.

### Results:

1H-NMR (400 MHz, CDCl3)-δ 5.83-5.73 (m, 1H), 5.05-4.96 (m, 2H), 4.07-4.03 (t, J=6.8 Hz, 2H), 2.38-2.30 (m, 2H), 2.11-2.06 (q, J=7.2 Hz, 2H), 1.77-1.68 (m, 2H), 1.64-1.57 (m, 2H), 1.30-1.21 (m, 16H), 0.87-0.83 (t, J=6.8 Hz, 3H) ppm.

### Synthesis of Intermediate [9]

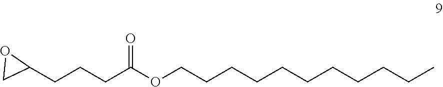 [SMILES: CCCCCCCCCCCOC(=O)CCCC1CO1]

As depicted in Scheme 4: To a stirred solution of undecyl hex-5-enoate [8](19.8 g, 73.8 mmol) in dichloromethane (200 mL), 3-chlorobenzene-1-carboperoxoic acid (25.5 g, 148 mmol) was added at 0° C. The resulting reaction mixture was stirred for 16 h at room temperature. The progress of reaction mass was monitored by TLC (SM was consumed). The resulting reaction mixture was washed with cold aqueous sodium bicarbonate solution (200 mL). The resulting organic layer was dried over Na₂SO₄, and concentrated under reduce pressure, and the crude was purified by flash column chromatography (SiO₂: 5-15% ethyl acetate in hexane), to give the desired undecyl 4-(oxiran-2-yl)butanoate [9](17.8 g, 84.84% Yield) as pale yellow liquid.

### Results:

1H-NMR (400 MHz, CDCl3)-δ 4.09-4.06 (t, J=6.8 Hz, 2H), 2.96-2.91 (m, 1H), 2.78-2.76 (t, J=4.8 Hz, 1H), 2.50-2.48 (m, 1H), 2.40-2.37 (m, 2H), 1.86-1.77 (m, 2H), 1.66-1.53 (m, 4H), 1.31-1.27 (m, 16H), 0.90-0.87 (t, J=6.8 Hz, 3H) ppm.

ELSD analysis: Purity 95.53%, Calculated C₁₇H₃₂O₃=284.24, Observed=285.20 (m/z, M+H+).

### Synthesis of Intermediate [11]

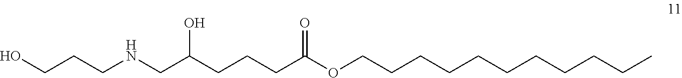 [SMILES: CCCCCCCCCCCOC(=O)CCCC(O)CNCCCO]

As depicted in Scheme 4: To a stirred solution of 3-aminopropan-1-ol [10](4.7 g, 62.6 mmol), and undecyl 4-(oxiran-2-yl)butanoate [9](17.8 g, 62.6 mmol) in isopropanol (100 mL was heated at 90° C. for 16 h. The progress of reaction was monitored by (SM was consumed). The reaction mixture was concentrated, and the crude was purified by flash column chromatography (SiO₂: 0-20% methanol in dichloromethane) to obtained the desired undecyl 5-hydroxy-6-[(3-hydroxypropyl)amino]hexanoate [11](6.5 g, 28.89% Yield) as light yellow liquid.

### Results:

ELSD analysis: Purity 99.56%, Calculated C₂₀H₄₁NO₄=359.30, Observed=360.65 (m/z, M+H+).

### Synthesis of Intermediate [12]

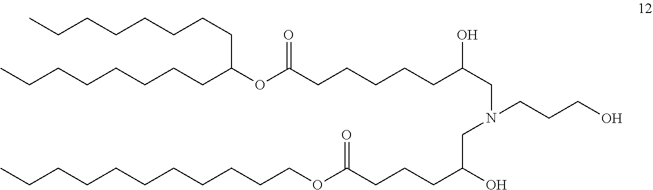 [SMILES: CCCCCCCCCCCOC(=O)CCCC(O)CN(CCCO)CC(O)CCCCCC(=O)OC(CCCCCCCC)CCCCCCCC]

As depicted in Scheme 4: To a stirred solution of undecyl 5-hydroxy-6-[(3-hydroxypropyl)amino]hexanoate [11](6.2 g, 17.2 mmol), and heptadecan-9-yl6-(oxiran-2-yl)hexanoate [5](7.52 g, 19 mmol) in isopropanol (100 mL) was heated at 90° C. for 16 h. The progress of reaction was monitored by (SM was consumed). The reaction mixture was concentrated, and the crude was purified by flash column chromatography (SiO₂: 0-20% methanol in dichloromethane) to obtain the desired heptadecan-9-yl 7-hydroxy-8-{[2-hydroxy-6-oxo-6-(undecyloxy)hexyl](3-hydroxypropyl)amino}octanoate [12](6.3 g, 48.31% Yield) as light yellow liquid.

### Results:

ELSD analysis: Purity 99.73%, Calculated C₄₅H₈₉NO₇=755.66, Observed=756.55 (m/z, M+H+).

### Synthesis of Compound LXXII

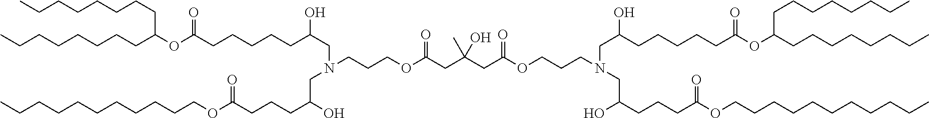 [SMILES: CCCCCCCCCCCOC(=O)CCCC(O)CN(CCCOC(=O)CC(C)(O)CC(=O)OCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC]

As depicted in Scheme 4: To a stirred solution of 3-hydroxy-3-methylpentanedioic acid [13](0.2 g, 1.23 mmol) and heptadecan-9-yl 7-hydroxy-8-{[2-hydroxy-6-oxo-6-(undecyloxy)hexyl](3-hydroxypropyl) amino}octanoate [12](1.77 g, 2.34 mmol) in dichloromethane (8 mL) was cooled to 0° C., EDC·HCl (709 mg, 3.7 mmol) was added followed by DMAP (456 mg, 3.7 mmol). The reaction mixture was stirred at r.t. for 48 h. The progress of reaction was monitored by ELSD/TLC (SM was consumed). Water (25 mL) was added to the reaction mixture and extract with DCM (3×50 mL). The resulting organic layer was dried over Na₂SO₄, and concentrated under reduce pressure, and the crude was purified by flash column chromatography (SiO₂: 0-5% methanol in dichloromethane), to obtain 1,5-bis(3-{[8-(heptadecan-9-yloxy)-2-hydroxy-8-oxooctyl][2-hydroxy-6-oxo-6-(undecyloxy)hexyl]amino}propyl) 3-hydroxy-3-methyl pentanedioate Compound LXXII (0.4 g, 19.79%, Yield) as a colorless liquid.

### Results:

1H NMR (400 MHz, CDCl3): δ 4.89-4.82 (m, 2H), 4.26-4.22 (m, 1H), 4.18-4.14 (m, 3H), 4.07-4.03 (t, J=6.4 Hz, 4H), 3.6 (brs, 4H), 3.28-3.26 (br, 2H), 2.75-2.60 (m, 7H), 2.58-2.52 (m, 2H), 2.49-2.39 (m, 4H), 2.37-2.31 (m, 4H), 2.30-2.26 (t, J=7.2 Hz, 4H), 1.80-1.79 (m, 6H), 1.67-1.57 (m, 14H), 1.50-1.49 (m, 8H), 1.45-1.39 (m, 6H), 1.36-1.25 (brs, 94H), 0.89-0.86 (t, J=7.2 Hz, 18H) ppm.

ELSD analysis: Purity 96.73%, Calculated C₉₆H₁₈₄N₂O₁₇=1637.36, Observed=1638.10 (m/z, M+H+).

### Example 5: Synthesis of Compound XXXVII

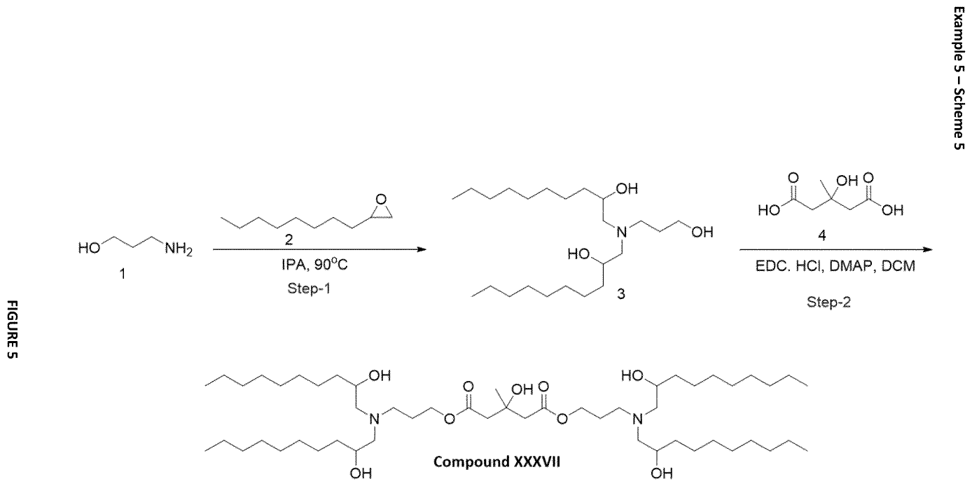

For example, the compounds of the invention may be prepared according to Scheme 5 (as depicted in FIG. 5).

### Synthesis of Intermediate [3]

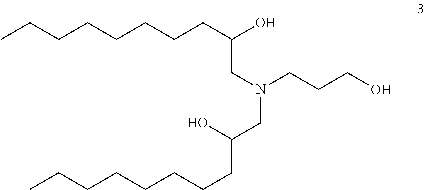 [SMILES: CCCCCCCCC(O)CN(CCCO)CC(O)CCCCCCCC]

As depicted in Scheme 5: A mixture of 2-octyloxirane [2](21.8 g, 140 mmol) and 3-aminopropan-1-ol [1](5 g, 66.6 mmol) in isopropanol (100 mL, 654 mmol) was heated under nitrogen atmosphere to 95° C. for 20 h. The progress of reaction was monitored by ELSD/TLC (SM was consumed). The reaction mixture was concentrated and the crude was purified by flash column chromatography (SiO₂: 0-20% methanol in dichloromethane) to obtain the desired 1-[(2-hydroxydecyl)(3-hydroxypropyl)amino]decan-2-ol [3](22 g, yield: 85%) as off white solid.

### Result:

ELSD analysis: Purity 99.85%, Calculated C₂₃H₄₉NO₃=387.37, Observed=388.35 (m/z, M+H+).

### Synthesis of Compound XXXVII

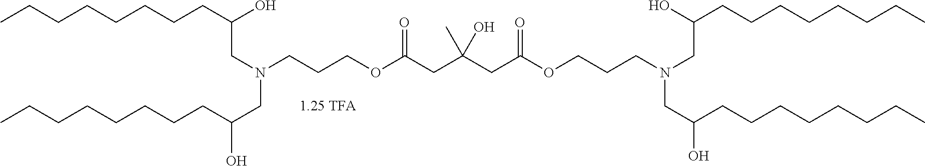 [SMILES: CCCCCCCCC(O)CN(CCCOC(=O)CC(C)(O)CC(=O)OCCCN(CC(O)CCCCCCCC)CC(O)CCCCCCCC)CC(O)CCCCCCCC]

As depicted in Scheme 5: To a stirred solution of 3-hydroxy-3-methylpentanedioic acid [4](0.1 g, 617 μmol) in dichloromethane (20 mL, 312 mmol), was added 4-(dimethylamino)pyridin-1-ium (456 mg, 3.7 mmol), {3-[cyano(ethyl)amino]propyl}dimethylazanium chloride (355 mg, 1.85 mmol) and 1-[(2-hydroxydecyl)(3-hydroxypropyl)amino]decan-2-ol [3](526 mg, 1.36 mmol) at RT under inert atmosphere. Resultant reaction allowed to stir at RT for 48 h. After 48 h reaction progress was monitor by TLC starting material was consumed completely. Reaction mass was evaporated under reduced pressure, then washed with heptane 5 time. Combined heptane fractions were evaporated under reduced pressure to gives crude reaction mass. Crude was purified by prep HPLC (ACN/0.1% TFA in water) as gradient eluent to give 1,5-bis({3-[bis(2-hydroxydecyl)amino]propyl}) 3-hydroxy-3-methylpentanedioate. Trifluoroacetic acid salt Compound XXXVII (0.14 g, 25% Yield) as Colorless liquid.

### Results:

1H NMR (400 MHz, CDCl3): δ 8.85-8.65 (brs, 1H), 8.55-8.35 (brs, 1H), 5.38-5.29 (m, 4H), 4.96 (s, 1H), 4.23-4.17 (m, 4H), 4.07 (s, 4H), 3.48-3.35 (m, 4H), 3.19-3.05 (m, 10H), 2.80-2.70 (m, 2H), 2.61-2.55 (m, 2H), 2.14 (s, 4H), 1.44-1.30 (m, 12H), 1.29-1.25 (brs, 45H), 0.89-0.86 (t, J=6.8 Hz, 12H) ppm.

ELSD analysis: Purity 99.92%, Calculated C₅₂H₁₀₄N₂O₉=900.77, Observed=901.60 (m/z, M+H+).

### Example 6: Synthesis of Compound XL

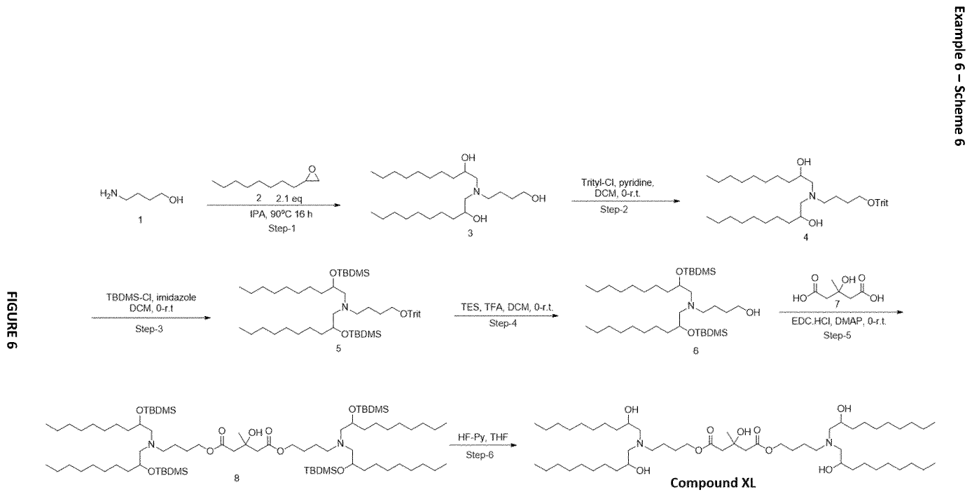

For example, the compounds of the invention may be prepared according to Scheme 6 (as depicted in FIG. 6).

### Synthesis of Intermediate [3]

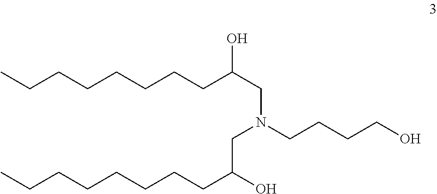 [SMILES: CCCCCCCCC(O)CN(CCCCO)CC(O)CCCCCCCC]

As depicted in Scheme 6: A mixture of 2-octyloxirane [2](18.4 g, 118 mmol) and 4-aminobutan-1-ol [1](5 g, 56.1 mmol) in isopropanol (100 mL) was stirred and heated under nitrogen atmosphere to 95° C. for 20 h. The progress of reaction was monitored by ELSD/TLC (SM was consumed). The reaction mixture was concentrated and the crude was purified by flash column chromatography (SiO₂: 0-10% Methanol in Dichloromethane) to obtain the desired 1-[(4-hydroxybutyl)(2-hydroxydecyl)amino]decan-2-ol [3](20.0 g, 91% Yield) as a white solid compound.

### Result:

ELSD analysis: Purity 99.85%, Calculated C₂₄H₅₁NO₃=401.39, Observed=402.45 (m/z, M+H+).

### Synthesis of Intermediate [4]

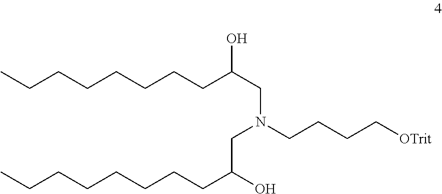

As depicted in Scheme 6: To a stirred solution of 1-[(4-hydroxybutyl)(2-hydroxydecyl)amino]decan-2-ol [3](5.0 g, 12.4 mmol) in dichloromethane (50 mL, 781 mmol), (chlorodiphenylmethyl)benzene (4.16 g, 14.9 mmol) and pyridine (985 mg, 12.4 mmol) was added in cooling, under inert atmosphere. The reaction mass was allowed to stir at RT for 16 h. Progress of reaction was monitored by TLC/ELSD. Reaction mass was dilute with DCM (50 mL), washed with fresh water (20 mL), extraction was done with DCM (3×50 mL). The organic layers were collect, dried over anhy. sodium sulphate, filtered, and concentrated under reduced pressure to get crude 1-[(2-hydroxydecyl)[4-(triphenylmethoxy)butyl]amino]decan-2-ol [4](8 g, 12.4 mmol) as colorless liquid, which was use as such for next step.

### Result:

ELSD analysis: Purity 97.95%, Calculated C₄₃H₆₅NO₃=643.50, Observed=644.45 (m/z, M+H+).

### Synthesis of Intermediate [5]

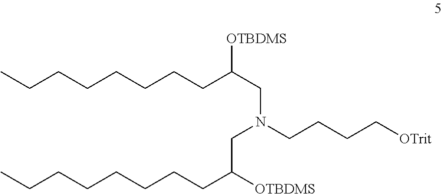

As depicted in Scheme 6: To a stirred solution of 1-[(2-hydroxydecyl)[4-(triphenylmethoxy)butyl]amino]decan-2-ol [4](8.0 g, 12.4 mmol) in dichloromethane (0.1 L, 1.56 mol), 1H-imidazole (7.61 g, 112 mmol) and tert-butyl(chloro)dimethylsilane (11.2 g, 74.5 mmol) was added successively under inert atmosphere. The resultant reaction mass allowed to stirred at RT for 16 h. Progress of reaction was monitor with TLC. SM was consumed completely. Reaction was quench with ice cold water (100.0 mL), extracted with DCM (2×50 ml). Combined organic layer was washed with brine and dried over sodium sulphate, filtered and evaporated under reduced pressure to gives crude reaction mass. Crude was purified with silica gel flash column chromatography using 30% Ethyl acetate in Hexane as gradient eluent to afford 2,2,3,3,11,11,12,12-octamethyl-5,9-dioctyl-7-[4-(triphenylmethoxy)butyl]-4,10-dioxa-7-aza-3,11-disilatridecane [5](10.7 g, 98.5% Yield; after two step) as colourless liquid.

### Result:

ELSD analysis: Purity 99.91%, Calculated C₅₅H₉₃NO₃Si₂=871.67, Observed=872.55 (m/z, M+H+).

### Synthesis of Intermediate [6]

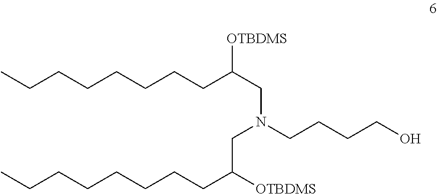 [SMILES: CCCCCCCCC(CN(CCCCO)CC(CCCCCCCC)O[Si](C)(C)C(C)(C)C)O[Si](C)(C)C(C)(C)C]

As depicted in Scheme 6: To the stirred solution of 2,2,3,3,11,11,12,12-octamethyl-5,9-dioctyl-7-[4-(triphenylmethoxy)butyl]-4,10-dioxa-7-aza-3,11-disilatridecane [5](5 g, 5.73 mmol) in dichloromethane (20 mL, 312 mmol), triethylsilyl (1.32 g, 11.5 mmol) was added at 0° C. followed by the addition of trifluoroacetic acid (3.27 g, 28.7 mmol) dropwise under inert atmosphere. The reaction was stirred at RT for 4 h. Progress of reaction was monitor by TLC. The reaction mass was quenched using saturated aq. NaHCO₃ solution up pH 8. The compound was extracted with DCM (2×100 mL). The combined organic layer was dried over anhy. Na₂SO₄, filtered and evaporated to get crude. The crude compound was purified on silica gel flash column chromatography by using 10-30% Ethyl acetate in Hexane as gradient eluent to afford 4-(2,2,3,3,11,11,12,12-octamethyl-5,9-dioctyl-4,10-dioxa-7-aza-3,11-disilatridecan-7-yl)butan-1-ol [6](3.0 g, 83% Yield) as a pale yellow liquid.

### Result:

ELSD analysis: Purity 99.48%, Calculated C₃₆H₇₉NO₃Si₂=629.56, Observed=630.55 (m/z, M+H+).

### Synthesis of Intermediate [8]

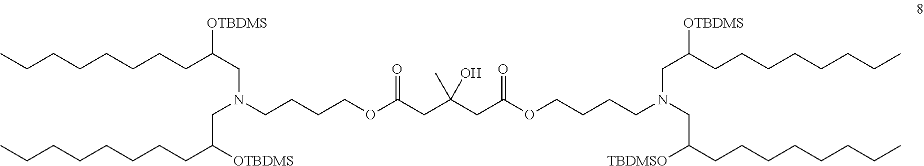 [SMILES: CCCCCCCCC(CN(CCCCOC(=O)CC(C)(O)CC(=O)OCCCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 6: A stirred solution of 4-(2,2,3,3,11,11,12,12-octamethyl-5,9-dioctyl-4,10-dioxa-7-aza-3,11-disilatridecan-7-yl)butan-1-ol [6](2.45 g, 3.89 mmol) and 3-hydroxy-3-methylpentanedioic acid [7](0.5 g, 3.4 mmol) in dichloromethane (50 mL, 781 mmol) was cooled to O ° C., {3-[cyano(ethyl)amino]propyl}dimethylazanium chloride (1.06 g, 5.55 mmol) was added followed by 4-(dimethylamino)pyridin-1-ium (684 mg, 5.55 mmol). The reaction mixture was stirred at RT for 48 h. The progress of reaction was monitored by ELSD/TLC. Water (50 mL) was added into the reaction mixture and extract with DCM (3×50 mL). The organic layer was collected, dried over Na₂SO₄, filtered and concentrated under reduce pressure. The crude was purified by flash silica column chromatography (0-100% ethyl acetate in hexane) as gradient eluent to give 1,5-bis[4-(2,2,3,3,11,11,12,12-octamethyl-5,9-dioctyl-4,10-dioxa-7-aza-3,11-disilatridecan-7-yl)butyl]3-hydroxy-3-methylpentanedioate [8](700 mg, 14.9%, Yield) as a pale yellow liquid.

### Result:

ELSD analysis: Purity 99.20%, Calculated C₇₃H₁₆₄N₂O₉Si₄=1385.15, Observed=1386.85 (m/z, M+H+).

### Synthesis of Compound XL

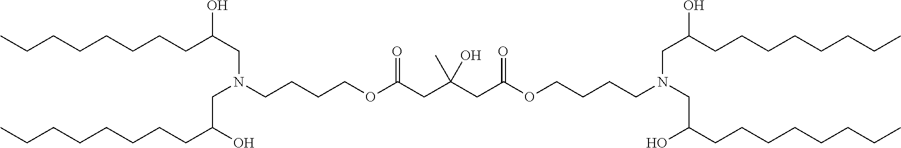 [SMILES: CCCCCCCCC(O)CN(CCCCOC(=O)CC(C)(O)CC(=O)OCCCCN(CC(O)CCCCCCCC)CC(O)CCCCCCCC)CC(O)CCCCCCCC]

As depicted in Scheme 6: To a stirred solution of 1,5-bis[4-(2,2,3,3,11,11,12,12-octamethyl-5,9-dioctyl-4,10-dioxa-7-aza-3,11-disilatridecan-7-yl)butyl]3-hydroxy-3-methylpentanedioate [8](0.7 g, 505 μmol) in tetrahydrofuran (5 mL, 61.4 mmol), slowly added pyridine hydrofluoride (0.3 g, 3.03 mmol) at 0° C. under inert atmosphere. Reaction mixture allowed to stir at RT for 16 h. Progress of reaction was monitor by TLC and ELSD data. After completion the reaction, reaction mass was quenched by saturated sodium bicarbonate up to pH 8. The extraction was done with ethyl acetate (3×50 ml). The combined organic layer was dried over sodium sulphate, filtered and evaporate under reduced pressure. The crude purified by with silica gel flash column to give 1,5-bis({4-[bis(2-hydroxydecyl)amino]butyl}) 3-hydroxy-3-methylpentanedioate Compound XL (126 mg, 26.8% Yield) as colorless liquid.

### Result:

1H NMR (400 MHz, CDCl3): δ 4.13-4.08 (m 4H), 3.65-3.60 (m, 4H), 2.71-2.53 (m, 8H), 2.49-2.38 (m, 8H), 1.70-1.58 (m, 4H), 1.56-1.49 (m, 4H), 1.48-1.43 (m, 4H), 1.42-1.33 (brs, 10H), 1.34-1.2 (brs, 45H), 0.89-0.86 (t, J=6.8 Hz, 12H) ppm.

ELSD analysis: Purity 99.35%, Calculated C₅₄H₁₀₈N₂O₉=928.81, Observed=929.60 (m/z, M+H+).

### Example 7: Synthesis of Compound XXXIX

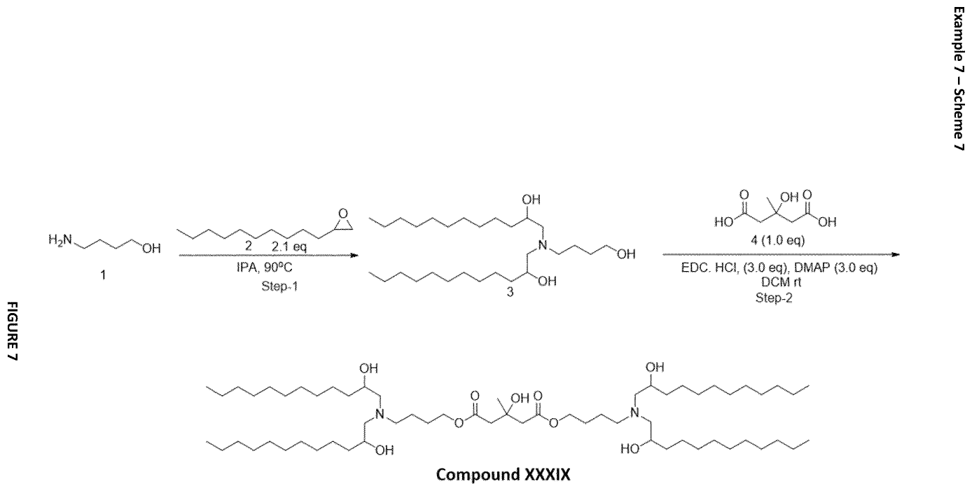

For example, the compounds of the invention may be prepared according to Scheme 7 (as depicted in FIG. 7).

### Synthesis of Intermediate [3]

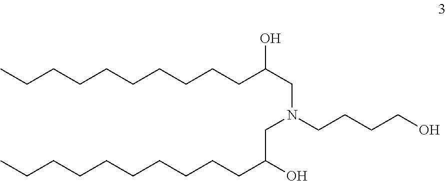 [SMILES: CCCCCCCCCCC(O)CN(CCCCO)CC(O)CCCCCCCCCC]

As depicted in Scheme 7: A solution of 4-aminobutan-1-ol [1](3 g, 33.7 mmol) and 2-decyloxirane [2](12.4 g, 67.3 mmol) in propan-2-ol (60 mL) was stirred at 90° C. for 20 h. Progress of reaction was monitored by TLC/ELSD. Reaction mixture was concentrated under reduced pressure and crude was purify by silica gel flash chromatography (0-7% methanol in dichloromethane) as gradient eluent to give 1-[(4-hydroxybutyl)(2-hydroxydodecyl)amino]dodecan-2-ol [3](11.2 g, 24.5 mmol) as pale yellow liquid.

### Result:

ELSD analysis: Purity 99.94%, Calculated C₂₈H₅₉NO₃=457.45, Observed=458.45 (m/z, M+H+).

### Synthesis of Compound XXXIX

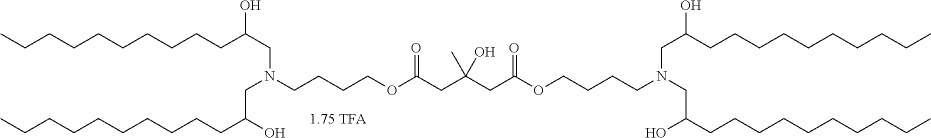 [SMILES: CCCCCCCCCCC(O)CN(CCCCOC(=O)CC(C)(O)CC(=O)OCCCCN(CC(O)CCCCCCCCCC)CC(O)CCCCCCCCCC)CC(O)CCCCCCCCCC]

As depicted in Scheme 7: To a stirred solution of 3-hydroxy-3-methylpentanedioic acid [4](0.4 g, 2.47 mmol) in dichloromethane (10 mL), was added {3-[cyano(ethyl)amino]propyl}dimethylazanium chloride (1.47 g, 7.65 mmol) and DMAP (942 mg, 7.65 mmol) followed by 1-[(4-hydroxybutyl)(2-hydroxydodecyl)amino]dodecan-2-ol [3](2.37 g, 5.18 mmol) at RT under inert atmosphere. The reaction mixture was stirred at RT for 48 h. Progress of reaction was monitor by TLC/ELSD. Reaction mass was evaporate under reduce pressure to give crude, which was purified by prep HPLC (acetonitrile/0.1% TFA in water) as gradient eluent to give 1,5-bis({4-[bis(2-hydroxydodecyl)amino]butyl}) 3-hydroxy-3-methylpentanedioate TFA salt Compound XXXIX (310 mg, 11.9% Yield) as a colorless semi solid.

### Result:

1H NMR (400 MHz, CDCl3): δ 4.14 (brs, 4H), 4.05 (brs, 4H), 3.68 (brs, 1H), 3.47-3.28 (m, 16H), 2.71-2.67 (m, 2H), 2.67-2.56 (m, 2H), 1.88 (brs, 4H), 1.73 (brs, 4H), 1.50-1.38 (m, 14H), 1.26 (brs, 61H), 0.89-0.86 (t, J=6.8 Hz, 12H) ppm.

ELSD analysis: Purity 99.84%, Calculated C₆₂H₁₂₄N₂O₉=1040.93, Observed=1041.75 (m/z, M+H+).

### Example 8: Synthesis of Compound XLV

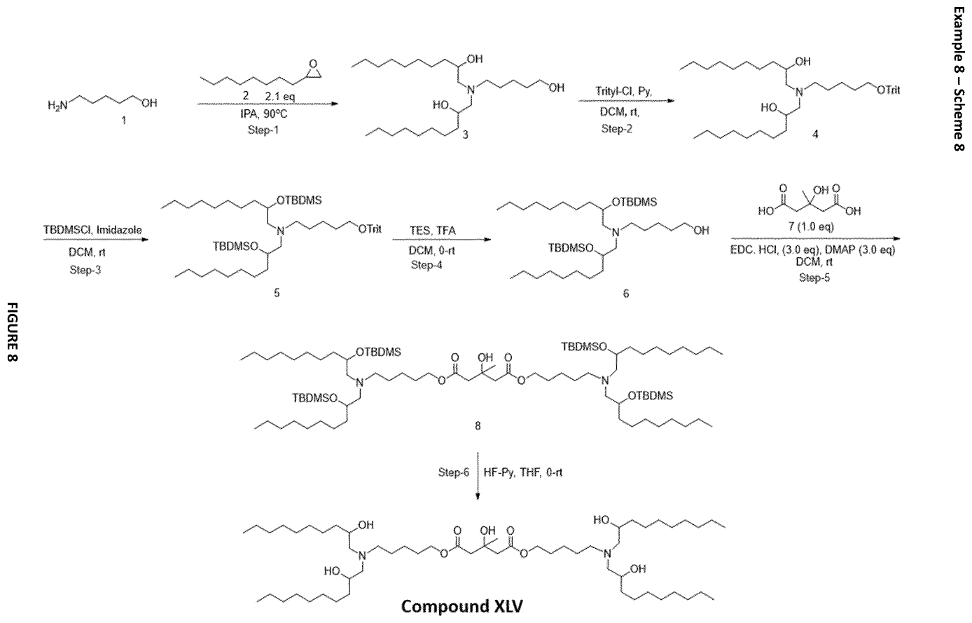

For example, the compounds of the invention may be prepared according to Scheme 8 (as depicted in FIG. 8).

### Synthesis of Intermediate [3]

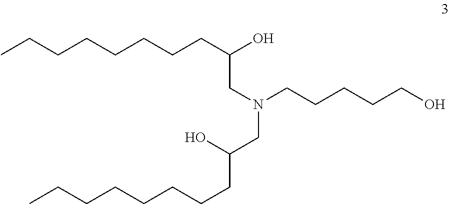 [SMILES: CCCCCCCCC(O)CN(CCCCCO)CC(O)CCCCCCCC]

As depicted in Scheme 8: To a stirred solution of 5-aminopentan-1-ol [1](1.0 g, 9.69 mmol) in isopropanol (20 mL) added 2-octyloxirane [2](3.33 g, 21.3 mmol) and allow to stirred at 90° C. for 16 h. Progress of reaction was monitor by TLC. After completion, reaction mixture was evaporated under reduce pressure to get crude, which was purified by silica gel flash column chromatography (0-5% MeOH in DCM) to give 1-[(2-hydroxydecyl)(5-hydroxypentyl)amino]decan-2-ol [3](2.65 g, 65.77% Yield) as greenish liquid.

### Result:

ELSD analysis: Purity 99.66%, Calculated C₂₅H₅₃NO₃=415.40, Observed=416.35 (m/z, M+H+).

### Synthesis of Intermediate [4]

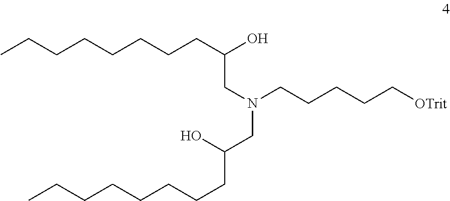

As depicted in Scheme 8: To a stirred solution of 1-[(2-hydroxydecyl)(5-hydroxypentyl)amino]decan-2-ol [3](2.4 g, 5.77 mmol) in DCM (25 mL), (chlorodiphenylmethyl)benzene (1.77 g, 6.35 mmol)) and pyridine (685 mg, 8.66 mmol) was added in cooling, under inert atmosphere. The reaction mass was allowed to stir at RT for 16 h. Progress of reaction was monitored by TLC/ELSD. Reaction mass was dilute with DCM (50 mL), washed with fresh water (50 mL). Organic layer was collect, dried over anhy. Sodium sulphate, filtered and concentrated to give crude 1-[(2-hydroxydecyl)[5-(triphenylmethoxy)pentyl]amino]decan-2-ol [4](3.8 g, crude) as colourless liquid, which was use as such for next step.

### Result:

ELSD analysis: Purity 97.79%, Calculated C₄₄H₆₇NO₃=657.51, Observed=658.45 (m/z, M+H+).

### Synthesis of Intermediate [5]

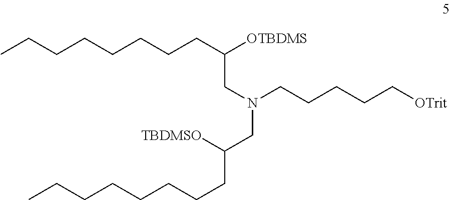

As depicted in Scheme 8: To a stirred solution of 1-[(2-hydroxydecyl)[5-(triphenylmethoxy)pentyl]amino]decan-2-ol [4](3.8 g, 5.77 mmol) in DCM (50 mL), added 1H-imidazole (2.36 g, 34.6 mmol) and tert-butyl(chloro)dimethylsilane (3.48 g, 23.1 mmol). Reaction mixture was stirred for 16 h at RT. Progress of reaction was monitor by TLC/ELSD. After completion, reaction mass was dilute with water (50 ml) and extracted with DCM (3×50 mL). Combined organic layer was dried over sodium sulphate, filtered and evaporated under reduce pressure to get crude, which was purified by silica gel flash column chromatography (30% Ethyl acetate in Hexane) to get 2,2,3,3,11,11,12,12-octamethyl-5,9-dioctyl-7-[5-(triphenylmethoxy)pentyl]-4,10-dioxa-7-aza-3,11-disilatridecane [5](2.8 g, 54.69% Yield after two step) as colorless liquid.

### Result:

ELSD analysis: Purity 99.96%, Calculated C₅₆H₉₅NO₃Si₂=885.69, Observed=886.55 (m/z, M+H+).

### Synthesis of Intermediate [6]

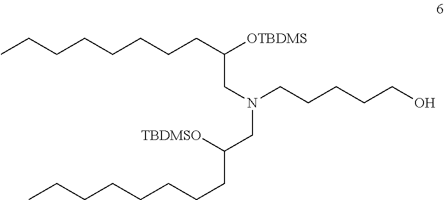 [SMILES: CCCCCCCCC(CN(CCCCCO)CC(CCCCCCCC)O[Si](C)(C)C(C)(C)C)O[Si](C)(C)C(C)(C)C]

As depicted in Scheme 8: To a stirred solution of 2,2,3,3,11,11,12,12-octamethyl-5,9-dioctyl-7-[5-(triphenylmethoxy)pentyl]-4,10-dioxa-7-aza-3,11-disilatridecane [5](2.8 g, 3.16 mmol) in dichloromethane (30 mL) was cool to 0° C., then trifluoroacetic acid (1.8 g, 15.8 mmol) and triethylsilyl (728 mg, 6.32 mmol) were added drop wise. The reaction mixture was stirred for 3 h at RT. Progress of reaction was monitored by TLC. After completion, reaction mass was quench by saturated aq. sodium bicarbonate up to pH 8, and extracted with DCM (3×50 ml). The organic layer was combine, dried over anhy. sodium sulphate, filtered and evaporated under reduce pressure to get crude, which was purified by silica gel flash column chromatography (10-30% ethyl acetate in hexane) to get 5-(2,2,3,3,11,11,12,12-octamethyl-5,9-dioctyl-4,10-dioxa-7-aza-3,11-disilatridecan-7-yl)pentan-1-ol [6](1.8 g, 88.47% Yield) as colourless liquid.

### Result:

ELSD analysis: Purity 99.31%, Calculated C₃₇H₃₁NO₃Si₂=643.58, Observed=644.50 (m/z, M+H+).

### Synthesis of Intermediate [8]

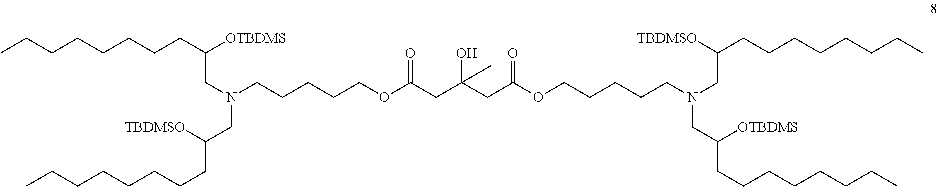 [SMILES: CCCCCCCCC(CN(CCCCCOC(=O)CC(C)(O)CC(=O)OCCCCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 8: To a stirred solution of 3-hydroxy-3-methylpentanedioic acid [7](0.2 g, 1.24 mmol) in dichloromethane (20 mL), added 5-(2,2,3,3,11,11,12,12-octamethyl-5,9-dioctyl-4,10-dioxa-7-aza-3,11-disilatridecan-7-yl)pentan-1-ol [6](1.59 g, 2.46 mmol), {3-[cyano(ethyl)amino]propyl}dimethylazanium chloride (710 mg, 3.70 mmol) and 4-(dimethylamino)pyridin-1-ium (456 mg, 3.70 mmol) at room temperature under inert atmosphere. Reaction mixture was stirred at RT for 48 h. Progress of reaction was monitored by TLC/ELSD. After completion, reaction mass was quench with water (20 mL) and extracted with DCM (3×50 mL). The organic layer was combine, dried over sodium sulphate, filter and evaporate under reduced pressure to get crude, which was purify by silica gel flash column chromatography (0-20% methanol in dichloromethane) to give 1,5-bis[5-(2,2,3,3,11,11,12,12-octamethyl-5,9-dioctyl-4,10-dioxa-7-aza-3,11-disilatridecan-7-yl)pentyl]3-hydroxy-3-methylpentanedioate [8](480 mg, 27.51% Yield) as colourless liquid.

### Result:

ELSD analysis: Purity 99.95%, Calculated C₃₀H₁₆₈N₂O₉Si₄=1413.18, Observed=1414.00 (m/z, M+H+).

### Synthesis of Compound XLV

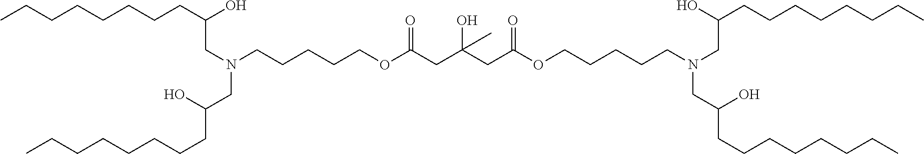 [SMILES: CCCCCCCCC(O)CN(CCCCCOC(=O)CC(C)(O)CC(=O)OCCCCCN(CC(O)CCCCCCCC)CC(O)CCCCCCCC)CC(O)CCCCCCCC]

As depicted in Scheme 8: To a stirred solution of 1,5-bis[5-(2,2,3,3,11,11,12,12-octamethyl-5,9-dioctyl-4,10-dioxa-7-aza-3,11-disilatridecan-7-yl)pentyl]3-hydroxy-3-methylpentanedioate [8](440 mg, 311 μmol) in tetrahydrofuran (4 mL), added pyridine hydro fluoride (308 mg, 3.11 mmol) drop wise at 0° C. Reaction mixture was allowed to stir for 16 h at RT. Progress of reaction was monitored by TLC/ELSD. After completion, reaction mass was quenched with aq. saturated sodium bicarbonate up to pH 8, and extracted with ethyl acetate (3×25 ml). The organic layer was combine, dried over sodium sulphate, filtered and evaporated under reduce pressure to get crude, which was purify by silica gel flash column chromatography (0-5% MeOH in DCM) to give 1,5-bis({5-[bis(2-hydroxydecyl)amino]pentyl}) 3-hydroxy-3-methylpentanedioate Compound XLV (0.145 g, 48.8% Yield) as pale yellow liquid.

### Result:

1H NMR (400 MHz, CDCl3): δ 4.15-4.08 (m, 5H), 3.64-3.63 (m, 4H), 2.70-2.55 (m, 8H), 2.45-2.39 (m, 8H), 1.69-1.62 (m, 4H), 1.50-1.27 (m, 66H), 0.88 (t, J=6.4 Hz, 12H) ppm.

ELSD analysis: Purity 99.85%, Calculated C₅₆H₁₁₂N₂O₉=956.84, Observed=957.60 (m/z, M+H+).

### Example 9: Synthesis of Compound XLVI

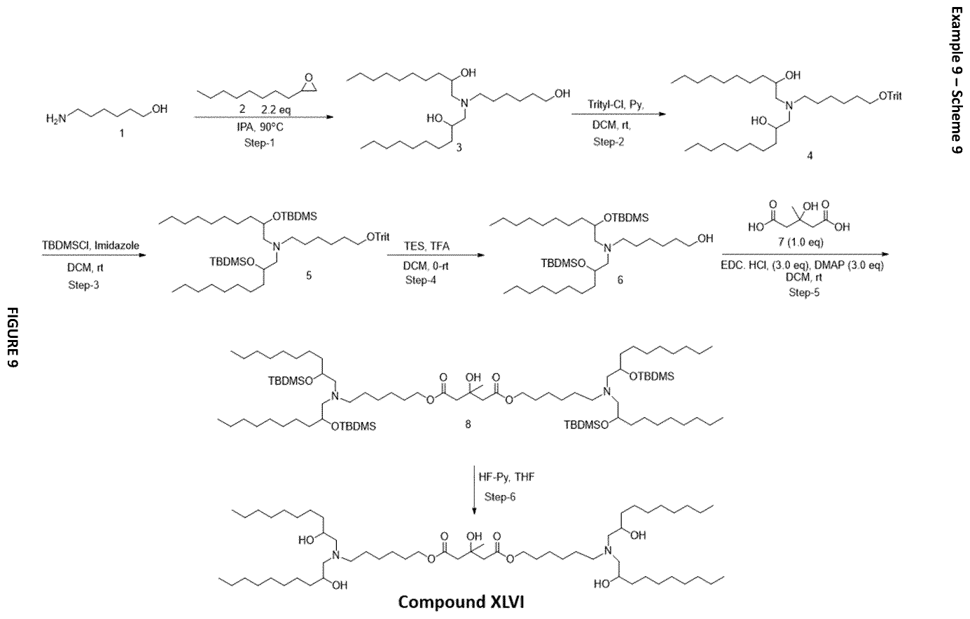

For example, the compounds of the invention may be prepared according to Scheme 9 (as depicted in FIG. 9).

### Synthesis of Intermediate [3]

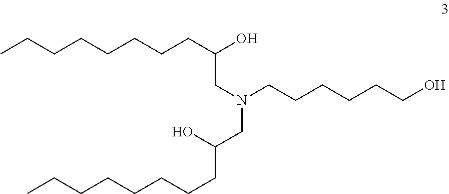 [SMILES: CCCCCCCCC(O)CN(CCCCCCO)CC(O)CCCCCCCC]

As depicted in Scheme 9: A mixture of 6-aminohexan-1-ol [1](1 g, 8.53 mmol) and 2-octyloxirane [2](2.93 g, 18.8 mmol) in isopropanol (20 mL, 262 mmol) was stirred and heated under nitrogen atmosphere at 95° C. for 20 h. The progress of reaction was monitored by ELSD/TLC (SM was consumed). The reaction mixture was concentrated, and the crude was purified by flash column chromatography (SiO₂: 0-20% methanol in dichloromethane) to give the desired 1-[(2-hydroxydecyl)(6-hydroxyhexyl)amino]decan-2-ol (3.0 g, 81.81%, Yield) as off white liquid.

### Results:

ELSD analysis: Purity 99.80%, Calculated C₂₆H₅₅NO₃=429.42, Observed=430.35 (m/z, M+H+).

### Synthesis of Intermediate [4]

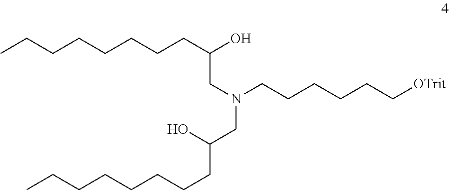

As depicted in Scheme 9: To stirred solution of 1-[(2-hydroxydecyl)(6-hydroxyhexyl)amino]decan-2-ol [3](2.5 g, 5.82 mmol) in DCM (30 mL) added pyridine (690 mg, 8.73 mmol) and (chlorodiphenylmethyl)benzene (1.78 g, 6.4 mmol) at RT. The reaction mixture was stirred for 16 h at RT. Reaction progress was monitored by TLC and ELSD data. After completion of reaction mixture, reaction mass evaporate under reduced pressure to get crude 1-[(2-hydroxydecyl)[6-(triphenylmethoxy)hexyl]amino]decan-2-ol [4](3.91 g; crude), which was use as such for next step.

### Results:

ELSD analysis: Purity 96.36%, Calculated C₄₅H₆₉NO₃=671.53, Observed=672.50 (m/z, M+H+).

### Synthesis of Intermediate [5]

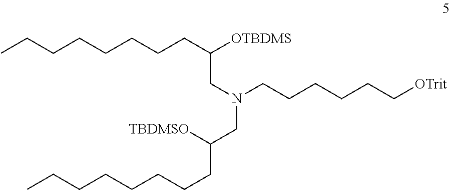

As depicted in Scheme 9: To stirred solution of 1-[(2-hydroxydecyl)[6-(triphenylmethoxy)hexyl]amino]decan-2-ol [4](3.91 g, 5.81 mmol) in DCM (50 mL), added 1H-imidazole (3.17 g, 46.54 mmol) and tert-butyl(chloro)dimethylsilane (5.26 g, 34.9 mmol) at RT. The reaction mixture was stirred for 16 h at RT. Reaction progress was monitored by TLC and ELSD data, after completion of reaction, reaction mass quenched with water (30 ml) and extracted with DCM (3×50 ml). Organic layer were combine and dried over sodium sulphate and evaporate under reduced pressure to get crude, which was purified over silica gel flash column chromatography by using 10-30% gradient of ethyl acetate in Hexane as eluent to get 2,2,3,3,11,11,12,12-octamethyl-5,9-dioctyl-7-[5-(triphenylmethoxy)pentyl]-4,10-dioxa-7-aza-3,11-disilatridecane [5](3.9 g, 74.56%, Yield after two step) as a light yellow liquid.

### Results:

ELSD analysis: Purity 99.67%, Calculated C₅₇H₉₇NO₃Si₂=899.70, Observed=900.55 (m/z, M+H+).

### Synthesis of Intermediate [6]

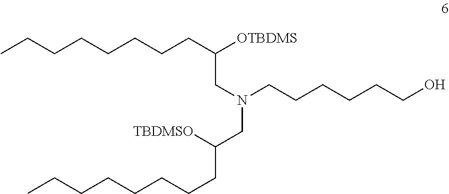 [SMILES: CCCCCCCCC(CN(CCCCCCO)CC(CCCCCCCC)O[Si](C)(C)C(C)(C)C)O[Si](C)(C)C(C)(C)C]

As depicted in Scheme 9: To a stirred solution of 2,2,3,3,11,11,12,12-octamethyl-5,9-dioctyl-7-[6-(triphenylmethoxy)hexyl]-4,10-dioxa-7-aza-3,11-disilatridecane [5](3.9 g, 4.33 mmol) in dichloromethane (53.2 mL, 831 mmol), added trifluoroacetic acid (2.47 g, 21.7 mmol) and triethylsilyl (1.37 mL, 8.66 mmol) drop wise simultaneously at cooling condition. The reaction mixture was stirred for 3 h at RT. Reaction progress was monitored by TLC, after completion of reaction, reaction mass quenched by saturated sodium bicarbonate and extracted with DCM (3×50 ml). The organic layer were combined, dried over sodium sulphate and evaporate under reduced pressure to get crude, which was purify over silica gel flash column chromatography by using 10-30% gradient of ethyl acetate in hexane as eluent to get 6-(2,2,3,3,11,11,12,12-octamethyl-5,9-dioctyl-4,10-dioxa-7-aza-3,11-disilatridecan-7-yl)hexan-1-ol [6](1.9 g, 66.65%, Yield) as a colourless liquid.

### Results:

ELSD analysis: Purity 99.67%, Calculated C₃₈H₈₃NO₃Si₂=657.59, Observed=658.50 (m/z, M+H+).

### Synthesis of Intermediate [8]

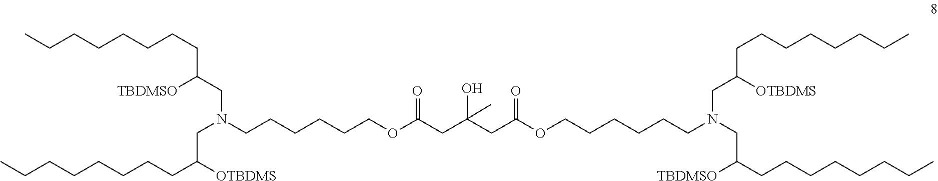 [SMILES: CCCCCCCCC(CN(CCCCCCOC(=O)CC(C)(O)CC(=O)OCCCCCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 9: To a stirred solution of 3-hydroxy-3-methylpentanedioic acid [7](234 mg, 1.44 mmol) in dichloromethane (30 mL, 469 mmol), was added 4-(dimethylamino)pyridin-1-ium (178 mg, 1.44 mmol) and {3-[cyano(ethyl)amino]propyl}dimethylazanium chloride (830 mg, 3 eq., 4.33 mmol) at room temperature. After this 6-(2,2,3,3,11,11,12,12-octamethyl-5,9-dioctyl-4,10-dioxa-7-aza-3,11-disilatridecan-7-yl)hexan-1-ol [6](1.9 g, 2 eq., 2.89 mmol) was added in reaction mixture and the reaction mixture was stirred for 32 h at RT. The reaction was monitored by TLC, after completion the reaction, reaction mixture was diluted with DCM and washed with brine solution. The organic layers were combined, dried over sodium sulphate, concentrated under reduced pressure to get crude, which was purify over silica gel flash column chromatography by using 10-30% gradient of ethyl acetate in hexane as eluent to get 1,5-bis[6-(2,2,3,3,11,11,12,12-octamethyl-5,9-dioctyl-4,10-dioxa-7-aza-3,11-disilatridecan-7-yl)hexyl]3-hydroxy-3-methylpentanedioate [8](390 mg, 18.83%, Yield) as pale yellow liquid.

### Results:

ELSD analysis: Purity 99.70%, Calculated C₈₂H₁₇₂N₂O₉Si₄=1441.21, Observed=1441.95 (m/z, M+H+).

### Synthesis of Compound XLVI

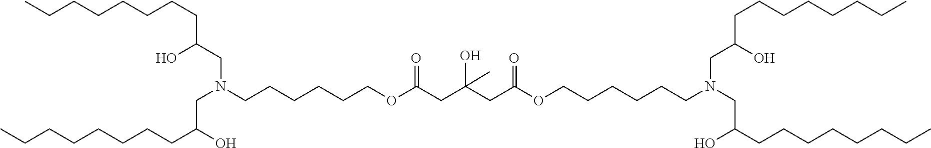 [SMILES: CCCCCCCCC(O)CN(CCCCCCOC(=O)CC(C)(O)CC(=O)OCCCCCCN(CC(O)CCCCCCCC)CC(O)CCCCCCCC)CC(O)CCCCCCCC]

As depicted in Scheme 9: To a stirred solution of 1,5-bis[6-(2,2,3,3,11,11,12,12-octamethyl-5,9-dioctyl-4,10-dioxa-7-aza-3,11-disilatridecan-7-yl)hexyl]3-hydroxy-3-methylpentanedioate [8](380 mg, 263 μmol) in tetrahydrofuran (5 mL, 61.4 mmol), pyridine hydrofluoride (400 μL, 4.48 mmol) was added at 0° C. The resulting reaction mixture was stirred for 16 h at room temperature. The progress of reaction was monitored by ELSD/TLC (SM was consumed). The resulting reaction mixture was quenched with cold aqueous sodium bicarbonate solution up to pH 8, and extract with ethyl acetate (3×20 mL). The resulting organic layer was dried over Na₂SO₄, filtered and concentrated under reduce pressure, and the crude was purified over flash column chromatography (SiO₂: 0-10% methanol in dichloromethane), to obtain 1,5-bis({6-[bis(2-hydroxydecyl)amino]hexyl}) 3-hydroxy-3-methylpentanedioate Compound XLVI (220 mg, 84.74%, Yield) as a pale yellow liquid.

### Results:

1H NMR (400 MHz, CDCl3): δ 4.14 (brs 1H), 4.11-4.08 (t, J=6.8 Hz, 4H), 3.66-3.65 (m, 4H), 2.71-2.57 (m, 8H), 2.48-2.42 (m, 8H), 1.66-1.62 (m, 4H), 1.54-1.37 (m, 17H), 1.35 (brs, 3H), 1.36-1.20 (m, 55H), 0.89-0.86 (t, J=7.2 Hz, 12H) ppm.

ELSD analysis: Purity 99.69%, Calculated C₅₈H₁₁₆N₂O₉=984.87, Observed=985.60 (m/z, M+H+).

### Example 10: Synthesis of Compound XLIII

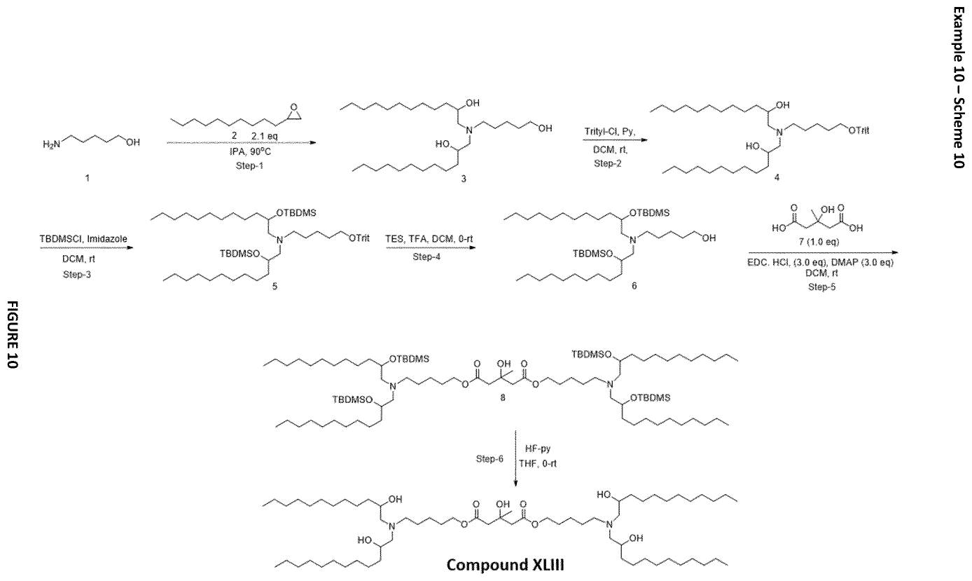

For example, the compounds of the invention may be prepared according to Scheme 10 (as depicted in FIG. 10).

### Synthesis of Intermediate [3]

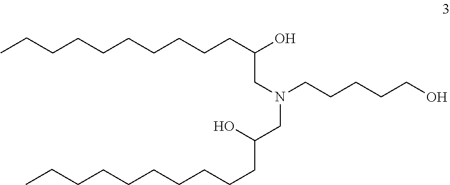 [SMILES: CCCCCCCCCCC(O)CN(CCCCCO)CC(O)CCCCCCCCCC]

As depicted in Scheme 10: To a stirred solution of 5-aminopentan-1-ol [1](1 g, 9.69 mmol) in isopropanol (20 mL) added 2-decyloxirane [2](3.93 g, 21.3 mmol) and the reaction mixture was stirred for 16 h at 90° C. Reaction progress was monitored by TLC, after completion of reaction, reaction mixture was evaporated under reduced pressure to get crude, and crude used for purification by flash column chromatography (0-10% Methanol in dichloromethane) to get 1-[(2-hydroxydodecyl)(5-hydroxypentyl)amino]dodecan-2-ol [3](3.5 g, 7.42 mmol) as a light green liquid.

### Result:

ELSD analysis: Purity 99.80%, Calculated C₂₉H₆₁NO₃=471.47, Observed=472.40 (m/z, M+H+).

### Synthesis of Intermediate [4]

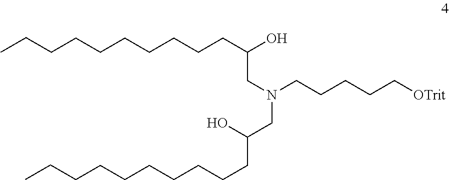

As depicted in Scheme 10: To a stirred solution of 1-[(2-hydroxydodecyl)(5-hydroxypentyl)amino]dodecan-2-ol [3](3.5 g, 7.42 mmol) in DCM (50 mL), added pyridine (880 mg, 11.1 mmol) and (chlorodiphenylmethyl)benzene (2.27 g, 8.16 mmol) at room temperature. The reaction mixture was stirred for 16 h at RT. Reaction progress was monitored by TLC and ELSD. After completion, reaction mass was evaporated under reduced pressure to get crude 1-[(2-hydroxydecyl)[6-(triphenylmethoxy)hexyl]amino]decan-2-ol [4](5.3 g crude). Crude was used as such for next step.

### Result:

ELSD analysis: Purity 98.10%, Calculated C₄₈H₇₅NO₃=713.57, Observed=714.55 (m/z, M+H+).

### Synthesis of Intermediate [5]

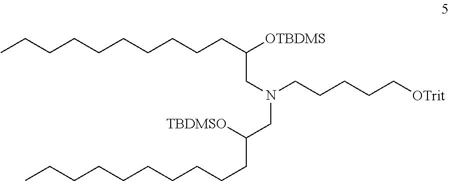

As depicted in Scheme 10: To a stirred solution of 1-[(2-hydroxydecyl)[6-(triphenylmethoxy)hexyl]amino]decan-2-ol [4](5.3 g, 7.42 mmol) in DCM (50 mL), added 1H-imidazole (3.03 g, 44.5 mmol) and tert-butyl(chloro)dimethylsilane (4.47 g, 29.7 mmol) at room temperature under inert atmosphere. The reaction mixture was stirred for 16 h at RT. Reaction progress was monitored by TLC and ELSD. After completion, reaction mass diluted with water (30 mL) and extracted with DCM (2×100 mL). Organic layer was combined and dried over sodium sulphate, evaporate under reduced pressure to get crude, which was purified by silica gel flash column chromatography (10-30% Ethyl acetate in Heptane) to get 5,9-bis(decyl)-2,2,3,3,11,11,12,12-octamethyl-7-[5-(triphenylmethoxy)pentyl]-4,10-dioxa-7-aza-3,11-disilatridecane [5](5.6 g, 80.05% Yield after two step) as yellow viscous oil.

### Result:

ELSD analysis: Purity 99.92%, Calculated C₆₀H₁₀₃NO₃Si₂=941.75, Observed=942.65 (m/z, M+H+).

### Synthesis of Intermediate [6]

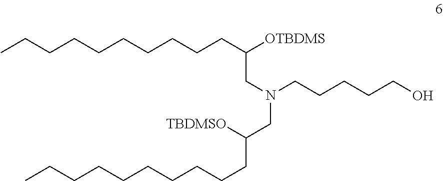 [SMILES: CCCCCCCCCCC(CN(CCCCCO)CC(CCCCCCCCCC)O[Si](C)(C)C(C)(C)C)O[Si](C)(C)C(C)(C)C]

As depicted in Scheme 10: To a stirred solution of 5,9-bis(decyl)-2,2,3,3,11,11,12,12-octamethyl-7-[5-(triphenylmethoxy)pentyl]-4,10-dioxa-7-aza-3,11-disilatridecane [5](5.6 g, 5.94 mmol) in dichloromethane (50 mL), simultaneously drop wise added trifluoroacetic acid (3.39 g, 29.7 mmol) and triethylsilyl (1.37 mL, 11.9 mmol) at cooling condition. The reaction mixture was stirred for 3 h at RT. Reaction progress was monitored by TLC. After completion of reaction, reaction mass quenched by saturated sodium bicarbonate up to pH 8, and extracted with DCM (2×100 ml). The organic layer were combined, dried over sodium sulphate and evaporate under reduced pressure to get crude, which was purified over silica gel flash column chromatography (10-30% Ethyl acetate in Hexane) to get 5-[5,9-bis(decyl)-2,2,3,3,11,11,12,12-octamethyl-4,10-dioxa-7-aza-3,11-disilatridecan-7-yl]pentan-1-ol [6](3.5 g, 84.13% Yield) as colorless liquid.

### Results:

ELSD analysis: Purity 99.24%, Calculated C₄₁H₈₉NO₃Si₂=699.64, Observed=700.55 (m/z, M+H+).

### Synthesis of Intermediate [8]

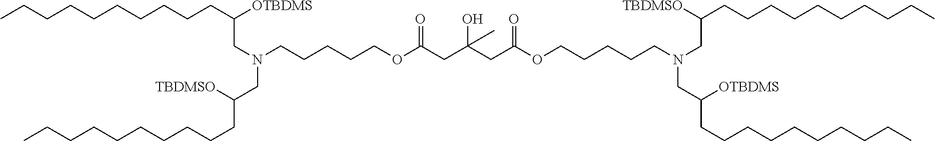 [SMILES: CCCCCCCCCCC(CN(CCCCCOC(=O)CC(C)(O)CC(=O)OCCCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 10: To a stirred solution of 3-hydroxy-3-methylpentanedioic acid [7](250 mg, 1.54 mmol) in dichloromethane (40 mL), was added 5-[5,9-bis(decyl)-2,2,3,3,11,11,12,12-octamethyl-4,10-dioxa-7-aza-3,11-disilatridecan-7-yl]pentan-1-ol [6](2.16 g 3.08 mmol), {3-[cyano(ethyl)amino]propyl}dimethylazanium chloride (0.877 g, 4.57 mmol) and DMAP (0.6 g, 4.87 mmol) at RT under inert atmosphere. The reaction mixture was stirred for for 48 h at RT. Reaction progress was monitored by TLC and ELSD. After completion of reaction, reaction mass quenched by water (20 mL) and extracted with DCM (3×25 ml). The organic layer were combined, dried over sodium sulphate, filtered and evaporate under reduced pressure to get crude, which was purified over silica gel flash column chromatography (0-40% Ethyl acetate in Heptane) to get desired product as 1,5-bis({5-[5,9-bis(decyl)-2,2,3,3,11,11,12,12-octamethyl-4,10-dioxa-7-aza-3,11-disilatridecan-7-yl]pentyl}) 3-hydroxy-3-methylpentanedioate [8](0.554 g, 23.6% Yield) as colourless liquid.

### Results:

ELSD analysis: Purity 99.86%, Calculated C₃₃H₁₈₄N₂O₉Si₄=1525.31, Observed=1526.15 (m/z, M+H+).

### Synthesis of Compound XLIII

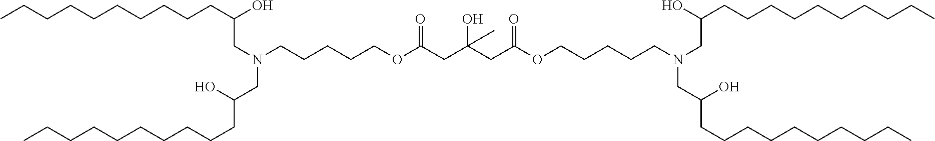 [SMILES: CCCCCCCCCCC(O)CN(CCCCCOC(=O)CC(C)(O)CC(=O)OCCCCCN(CC(O)CCCCCCCCCC)CC(O)CCCCCCCCCC)CC(O)CCCCCCCCCC]

As depicted in Scheme 10: To a stirred solution of 1,5-bis({5-[5,9-bis(decyl)-2,2,3,3,11,11,12,12-octamethyl-4,10-dioxa-7-aza-3,11-disilatridecan-7-yl]pentyl}) 3-hydroxy-3-methylpentanedioate [8](0.5 g, 327 μmol) in tetrahydrofuran (7 mL), added pyridine hydrofluoride (0.3 mL, 3.27 mmol) drop wise at cooling condition then allowed to stir for 16 h at RT. Reaction progress was monitor by TLC and ELSD data. After completion, reaction mass was quenched by saturated sodium bicarbonate up to pH 8, and extracted with ethyl acetate (3×25 ml). The organic layer was combined, dried over sodium sulphate, filtered and evaporate under reduce pressure to get crude, which was purify over silica (0-10% MeOH in DCM) to give 1,5-bis({5-[bis(2-hydroxydodecyl)amino]pentyl}) 3-hydroxy-3-methylpentanedioate Compound XLIII (0.2 g, 57.09% Yield) as colourless liquid.

### Results:

1H NMR (400 MHz, CDCl3): δ 4.16 (brs, 1H), 4.11-4.08 (t, J=6.4 Hz, 4H), 3.64-3.63 (m, 4H), 2.71-2.55 (m, 11H), 2.45-2.39 (m, 10H), 1.69-1.60 (m, 4H), 1.50-1.43 (m, 16H), 1.40-1.33 (m, 5H), 1.26 (m, 61H), 0.89-0.86 (t, J=7.2 Hz, 12H) ppm.

ELSD analysis: Purity 99.87%, Calculated C₆₄H₁₂₈N₂O₉=1068.96, Observed=1069.70 (m/z, M+H+).

### Example 11: Synthesis of Compound XLIV

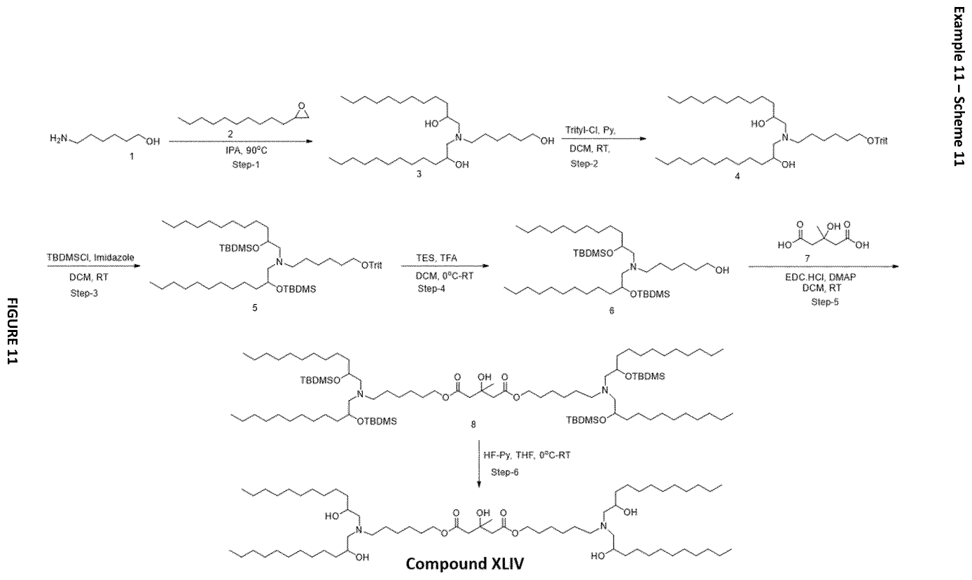

For example, the compounds of the invention may be prepared according to Scheme 11 (as depicted in FIG. 11).

### Synthesis of Intermediate [3]

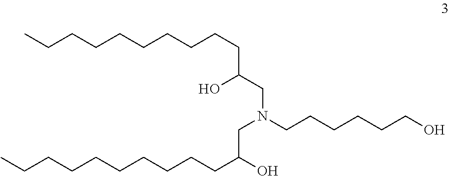 [SMILES: CCCCCCCCCCC(O)CN(CCCCCCO)CC(O)CCCCCCCCCC]

As depicted in Scheme 11: To a stirred solution of 6-aminohexan-1-ol [1](1 g, 8.53 mmol) in isopropanol (20 mL) was added 2-decyloxirane [2](3.46 g, 2.2 eq., 18.8 mmol). The reaction mixture was stirred for 16 h 90° C. Progress of reaction was monitor by TLC. After completion, reaction mixture was evaporated under reduced pressure to get crude, which was purified by silica gel flash column chromatography (0-5% MeOH in DCM) to give 1-[(2-hydroxydodecyl)(6-hydroxyhexyl) amino]dodecan-2-ol [3](3 g, 72% Yield) as greenish liquid.

### Result:

ELSD analysis: Purity 99.76%, Calculated C₃₀H₆₃NO₃=485.48, Observed=486.50 (m/z, M+H+).

### Synthesis of Intermediate [4]

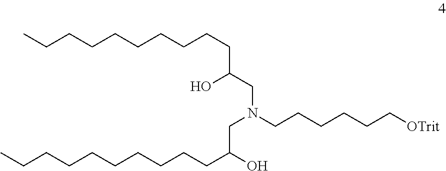

As depicted in Scheme 11: To a stirred solution of 1-[(2-hydroxydodecyl)(6-hydroxyhexyl)amino]dodecan-2-ol [3](2.8 g, 5.76 mmol) in DCM (25 mL), (chlorodiphenylmethyl)benzene (1.61 g, 5.76 mmol)) and pyridine (684 mg, 8.64 mmol) added at 0° C., under inert atmosphere. The reaction mass allowed to stir at RT for 16 h. Progress of reaction was monitored by TLC/ELSD. Reaction mass was dilute with DCM (50 mL), washed with fresh water (50 mL). Organic layer was collect, dried over anhy. Sodium sulphate, filtered and concentrated to give of 1-[(2-hydroxydodecyl)[6-(triphenylmethoxy)hexyl]amino]dodecan-2-ol [4](4.2 g, crude) as colourless liquid. Compound was use as such for next step.

### Result:

ELSD analysis: Purity 97.60%, Calculated C₄₉H₇₇NO₃=727.59, Observed=728.50 (m/z, M+H+).

### Synthesis of Intermediate [5]

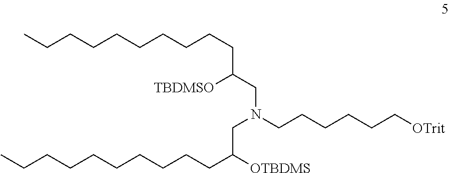

As depicted in Scheme 11: To a stirred solution of 1-[(2-hydroxydodecyl)[6-(triphenylmethoxy)hexyl]amino]dodecan-2-ol [4](4.2 g, 5.77 mmol) in DCM (50 mL), added 1H-imidazole (2.36 g, 34.6 mmol) and tert-butyl(chloro)dimethylsilane (3.48 g, 23.1 mmol). The reaction mixture was stirred for 16 h at RT. Progress of reaction was monitored by TLC/ELSD. After completion, reaction mass was dilute with water (100 ml) and extracted with DCM (3×50 mL). Combined organic layer was dried over sodium sulphate, filtered and evaporated under reduced pressure to get crude, which was purified by silica gel flash column chromatography (0-30% Ethyl acetate in Hexane) to get 5,9-bis(decyl)-2,2,3,3,11,11,12,12-octamethyl-7-[6-(triphenylmethoxy)hexyl]-4,10-dioxa-7-aza-3,11-disilatridecane [5](4.79 g, 86.9% Yield after two step) as colourless liquid.

### Result:

ELSD analysis: Purity 98.64%, Calculated Ce₁H₁₀₅NO₃Si₂=955.76, Observed=956.70 (m/z, M+H+).

### Synthesis of Intermediate [6]

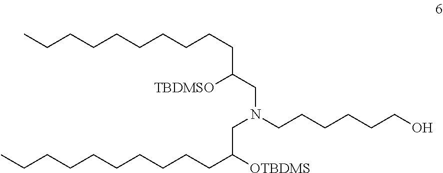 [SMILES: CCCCCCCCCCC(CN(CCCCCCO)CC(CCCCCCCCCC)O[Si](C)(C)C(C)(C)C)O[Si](C)(C)C(C)(C)C]

As depicted in Scheme 11: A stirred solution of 5,9-bis(decyl)-2,2,3,3,11,11,12,12-octamethyl-7-[6-(triphenylmethoxy)hexyl]-4,10-dioxa-7-aza-3,11-disilatridecane [5](4.79 g, 5.0 mmol) in dichloromethane (50 mL) was cool to 0° C., then trifluoroacetic acid (2.85 g, 25.0 mmol) and triethylsilyl (1.15 g, 10.0 mmol) were added drop wise. The reaction mixture was stirred for 3 h at RT. Progress of reaction was monitored by TLC. After completion, reaction mass was quenched by saturated aq. sodium bicarbonate up to pH 8 and extracted with DCM (3×50 mL). The organic layer was combined, dried over anhy. sodium sulphate, filtered and evaporated under reduced pressure to get crude, which was purified by silica gel flash column chromatography (0-30% ethyl acetate in hexane) to get 6-[5,9-bis(decyl)-2,2,3,3,11,11,12,12-octamethyl-4,10-dioxa-7-aza-3,11-disilatridecan-7-yl]hexan-1-ol [6](2.9 g, 81.1% Yield) as colorless liquid.

### Result:

ELSD analysis: Purity 98.98%, Calculated C₄₂H₉₁NO₃Si₂=713.65, Observed=714.60 (m/z, M+H+).

### Synthesis of Intermediate [8]

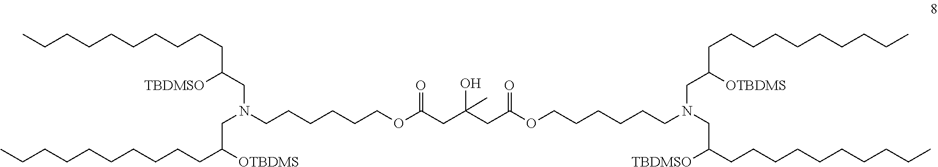 [SMILES: CCCCCCCCCCC(CN(CCCCCCOC(=O)CC(C)(O)CC(=O)OCCCCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 11: To a stirred solution of 3-hydroxy-3-methylpentanedioic acid [7](0.3 g, 1.85 mmol) in dichloromethane (20 mL), added 6-[5,9-bis(decyl)-2,2,3,3,11,11,12,12-octamethyl-4,10-dioxa-7-aza-3,11-disilatridecan-7-yl]hexan-1-ol [6](2.64 g, 3.69 mmol), {3-[cyano(ethyl)amino]propyl}dimethylazanium chloride (1.06 g, 5.55 mmol) and DMAP (684 mg, 5.55 mmol) at room temperature under inert atmosphere. The reaction mixture was stirred at RT for 48 h. Progress of reaction was monitored by TLC/ELSD. After completion, reaction mass was quenched with water (20 mL) and extracted with DCM (3×50 mL). The organic layer was combined, dried over sodium sulphate, filtered and evaporated under reduced pressure to get crude, which was purified by silica gel flash column chromatography (0-20% methanol in dichloromethane) to give 1,5-bis({6-[5,9-bis(decyl)-2,2,3,3,11,11,12,12-octamethyl-4,10-dioxa-7-aza-3,11-disilatridecan-7-yl]hexyl}) 3-hydroxy-3-methylpentanedioate [8](388 mg, 13.5% Yield) as colorless liquid.

### Result:

ELSD analysis: Purity 99.83%, Calculated C₉₀H₁₃₃N₂O₉Si₄=1553.34, Observed=1554.25 (m/z, M+H+).

### Synthesis of Compound XLIV

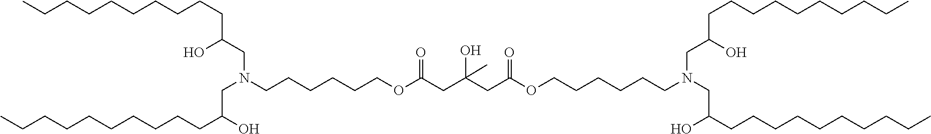 [SMILES: CCCCCCCCCCC(O)CN(CCCCCCOC(=O)CC(C)(O)CC(=O)OCCCCCCN(CC(O)CCCCCCCCCC)CC(O)CCCCCCCCCC)CC(O)CCCCCCCCCC]

As depicted in Scheme 11: To a stirred solution of 1,5-bis[5-(2,2,3,3,11,11,12,12-octamethyl-5,9-dioctyl-4,10-dioxa-7-aza-3,11-disilatridecan-7-yl)pentyl]3-hydroxy-3-methylpentanedioate [8](388 mg, 0.249 mmol) in tetrahydrofuran (4 mL), added pyridine hydro fluoride (247 mg, 2.49 mmol) drop wise at 0° C. The reaction mixture was stirred for 16 h at RT. Progress of reaction was monitored by TLC/ELSD. After completion, reaction mass was quenched by aq. saturated sodium bicarbonate up to pH 8, and extracted with ethyl acetate (3×25 ml). The organic layer was combined, dried over sodium sulphate, filtered and evaporated under reduced pressure to get crude, which was purify by silica gel flash column chromatography (0-5% MeOH in DCM) to give 1,5-bis({6-[bis(2-hydroxydodecyl)amino]hexyl}) 3-hydroxy-3-methylpentanedioate Compound XLIV (0.16 g, 58.6% Yield) as pale yellow liquid.

### Result:

1H NMR (400 MHz, CDCl3): δ 4.28-4.14 (brs, 1H), 4.11-4.08 (t, J=6.4 Hz, 4H), 3.65-3.64 (m, 4H), 2.71-2.57 (m, 9H), 2.47-2.40 (m, 7H), 1.67-1.60 (m, 4H), 1.48-1.33 (m, 26H), 1.32-1.26 (brs, 61H), 0.89-0.86 (t, J=6.4 Hz, 12H) ppm.

ELSD analysis: Purity 99.15%, Calculated C₆₆H₁₃₂N₂O₉=1096.99, Observed=1097.80 (m/z, M+H+).

### Example 12: Synthesis of Compound XLVII

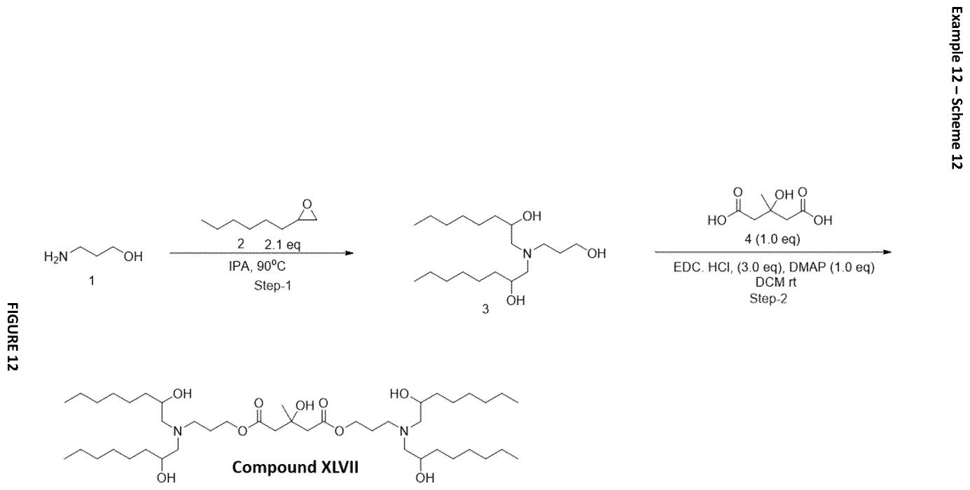

For example, the compounds of the invention may be prepared according to Scheme 12 (as depicted in FIG. 12).

### Synthesis of Intermediate [3]

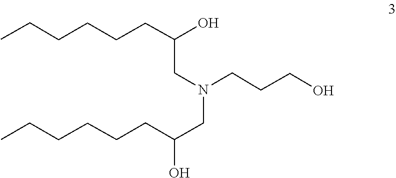 [SMILES: CCCCCCC(O)CN(CCCO)CC(O)CCCCCC]

As depicted in Scheme 12: A mixture of 2-hexyloxirane [2](3.6 g, 28 mmol) and 3-aminopropan-1-ol [1](1 g, 13.3 mmol) in isopropanol (20 mL, 654 mmol) was stirred and heated under nitrogen atmosphere to 95° C. for 20 h. The progress of reaction was monitored by ELSD/TLC (SM was consumed). The reaction mixture was concentrated and the crude was purified by flash column chromatography (SiO₂: 0-20% methanol in dichloromethane) to obtain the desired 1-[(2-hydroxyoctyl)(3-hydroxypropyl)amino]octan-2-ol (2.8 g, 63.49% Yield) as colourless liquid.

ELSD analysis: Purity 99.80%, Calculated C₁₉H₄₁NO₃=331.31, Observed=332.30 (m/z, M+H+).

### Synthesis of Compound XLVII

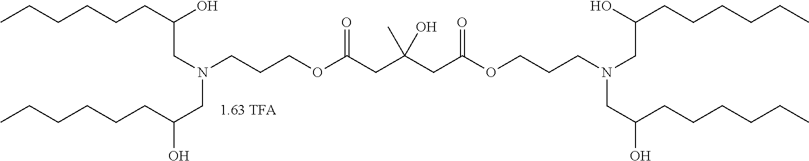 [SMILES: CCCCCCC(O)CN(CCCOC(=O)CC(C)(O)CC(=O)OCCCN(CC(O)CCCCCC)CC(O)CCCCCC)CC(O)CCCCCC]

As depicted in Scheme 12: To a stirred solution of 3-hydroxy-3-methylpentanedioic acid [4](0.2 g, 1.23 mmol) in dichloromethane (20 mL), was added 4-(dimethylamino)pyridin-1-ium (456 mg, 3.7 mmol), {3-[cyano(ethyl)amino]propyl}dimethylazanium chloride (919 mg, 4.79 mmol)) and 1-[(2-hydroxyoctyl)(3-hydroxypropyl)amino]octan-2-ol [3](859 mg, 2.59 mmol) at RT under inert atmosphere. Resultant reaction mass allowed to stir at RT for 48 h. Reaction progress was monitor by TLC/ELSD. Reaction mass was evaporated under reduced pressure, then washed with heptane 5 time. Combined heptane fractions were evaporated under reduced pressure to give crude reaction mass. Crude was purified by prep HPLC (ACN/0.1% TFA in water) to give 1,5-bis({3-[bis(2-hydroxyoctyl)amino]propyl}) 3-hydroxy-3-methylpentanedioate trifluoroacetic acid salt Compound XLVII (325 mg, 33.4% Yield) as colourless liquid.

### Results:

1H NMR (400 MHz, CDCl3): δ 4.36-4.16 (m, 4H), 4.10-4.05 (m, 4H), 3.68-3.30 (m, 6H), 3.30-2.95 (m, 10H), 2.80-2.67 (m, 3H), 2.61-2.53 (m, 3H), 2.12-2.08 (brs, 4H), 1.60-1.39 (m, 14H), 1.35-1.22 (brs, 28H), 0.89-0.86 (t, J=6.8 Hz, 12H) ppm.

ELSD analysis: Purity 99.81%, Calculated C₄₄H₈₈N₂O₉=788.65, Observed=789.50 (m/z, M+H+).

### Example 13: Synthesis of Compound XLVIII

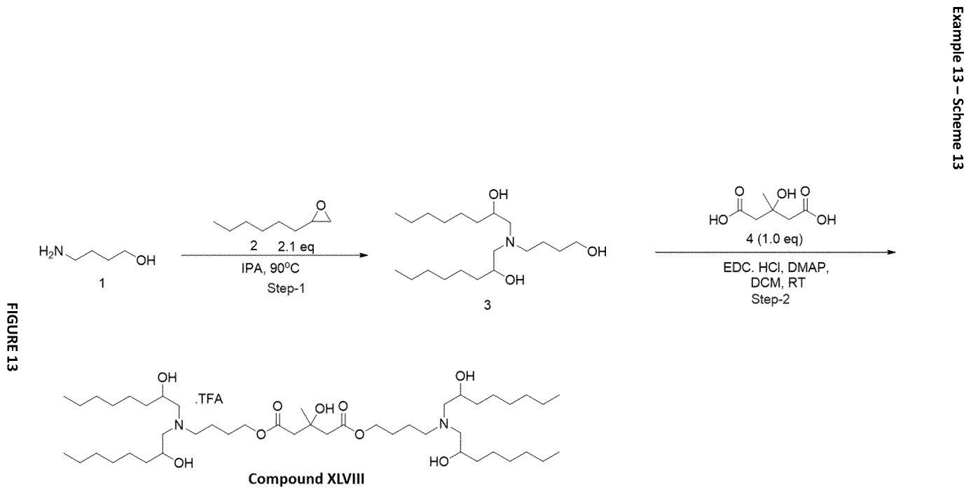

For example, the compounds of the invention may be prepared according to Scheme 13 (as depicted in FIG. 13).

### Synthesis of Intermediate [3]

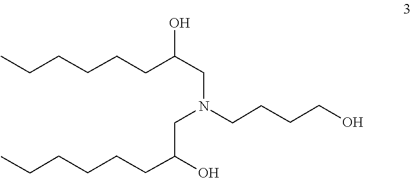 [SMILES: CCCCCCC(O)CN(CCCCO)CC(O)CCCCCC]

As depicted in Scheme 13: A mixture of 2-hexyloxirane [2](3.6 g, 28 mmol) and 4-aminobutan-1-ol [1](1 g, 11.2 mmol) in isopropanol (20 mL, 654 mmol) was stirred and heated under nitrogen atmosphere to 95° C. for 20 h. The progress of reaction was monitored by ELSD/TLC (SM was consumed). The reaction mixture was concentrated and the crude was purified by flash column chromatography (SiO₂: 0-20% methanol in dichloromethane) to obtained the desired 1-[(4-hydroxybutyl)(2-hydroxyoctyl)amino]octan-2-ol [3](3.4 g, 87.7% Yield) as colourless liquid.

### Result:

ELSD analysis: Purity 99.80%, Calculated C₂₀H₄₃NO₃=345.32, Observed=346.35 (m/z, M+H+).

### Synthesis of Compound XLVIII

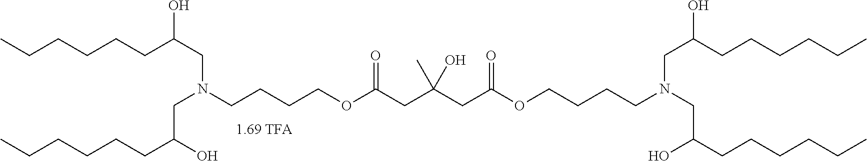 [SMILES: CCCCCCC(O)CN(CCCCOC(=O)CC(C)(O)CC(=O)OCCCCN(CC(O)CCCCCC)CC(O)CCCCCC)CC(O)CCCCCC]

As depicted in Scheme 13: To a stirred solution of 3-hydroxy-3-methylpentanedioic acid [4](0.4 g, 2.47 mmol) in dichloromethane (25 mL), was added {3-[cyano(ethyl)amino]propyl}dimethylazanium chloride (1.89 g, 9.87 mmol) and 4-(dimethylamino)pyridin-1-ium (1.22 g, 9.87 mmol) and 1-[(4-hydroxybutyl)(2-hydroxyoctyl)amino]octan-2-ol [3](1.62 g, 4.69 mmol) at RT under inert atmosphere. The reaction mixture was stirred for 48 h at RT. Reaction progress was monitored by TLC/ELSD. Reaction mass was evaporated under reduced pressure, then washed with heptane 5 times. Combined heptane fractions were evaporated under reduced pressure to give crude reaction mass. Crude was purified by prep HPLC (ACN/0.1% TFA in water) to give 1,5-bis({4-[bis(2-hydroxyoctyl)amino]butyl}) 3-hydroxy-3-methylpentanedioate trifluoroacetic acid salt (Compound XLVIII; 256 mg, 12.7% Yield) as light yellow semi solid.

### Results:

1H-NMR (400 MHz, CDCl3)-δ 4.21-3.90 (m, 8H), 3.40-3.25 (m, 4H), 3.22-2.95 (m, 8H), 2.73-2.67 (m, 2H), 2.60-2.56 (m, 2H), 1.92-1.81 (m, 4H), 1.80-1.69 (m, 4H), 1.57-1.40 (m, 11H), 1.38 (s, 3H), 1.33-1.22 (m, 29H), 0.89-0.86 (t, J=6.8 Hz, 12H) ppm.

ELSD analysis: Purity 99.95%, Calculated C₄₆H₉₂N₂O₉=816.68, Observed=817.50 (m/z, M+H+).

### Example 14: Synthesis of Compound XLIX

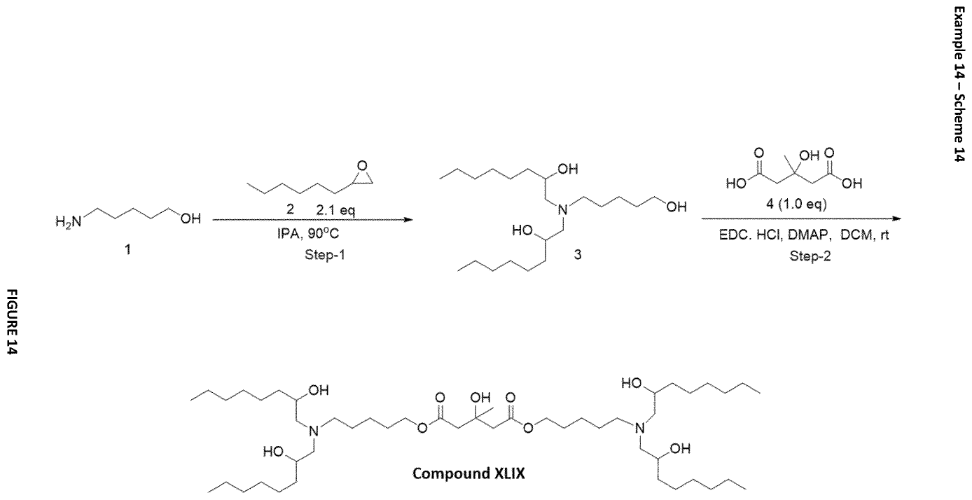

For example, the compounds of the invention may be prepared according to Scheme 14 (as depicted in FIG. 14).

### Synthesis of Intermediate [3]

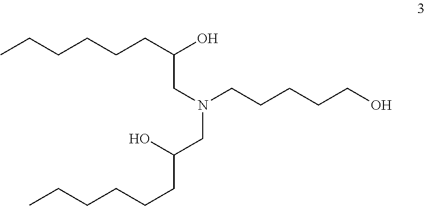 [SMILES: CCCCCCC(O)CN(CCCCCO)CC(O)CCCCCC]

As depicted in Scheme 14: A solution of 5-aminopentan-1-ol [1](1 g, 9.69 mmol) and 2-hexyloxirane [2](3.11 g, 24.2 mmol) in isopropanol (20 mL, 654 mmol) was stirred under nitrogen atmosphere to 95° C. for 20 h. The progress of reaction was monitored by ELSD/TLC (SM was consumed). The reaction mixture was concentrated and the crude was purified by flash column chromatography (SiO₂: 0-20% methanol in dichloromethane) to afford 1-[(2-hydroxyoctyl)(5-hydroxypentyl) amino]octan-2-ol [3](3.1 g, 88.94% Yield) as colourless liquid.

### Result:

ELSD analysis: Purity 99.69%, Calculated C₂₁H₄₅NO₃=359.34, Observed=360.40 (m/z, M+H+).

### Synthesis of Compound XLIX

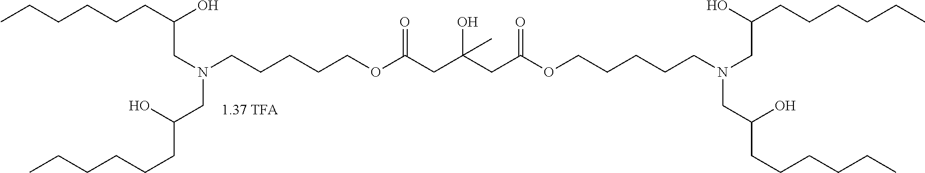 [SMILES: CCCCCCC(O)CN(CCCCCOC(=O)CC(C)(O)CC(=O)OCCCCCN(CC(O)CCCCCC)CC(O)CCCCCC)CC(O)CCCCCC]

As depicted in Scheme 14: To a stirred solution of 3-hydroxy-3-methylpentanedioic acid [4](0.4 g, 2.47 mmol) in dichloromethane (10 mL), added {3-[cyano(ethyl)amino]propyl}dimethylazanium chloride (1.89 g, 9.87 mmol) and 4-(dimethylamino)pyridin-1-ium (1.22 g, 9.87 mmol) and 1-[(2-hydroxyoctyl)(5-hydroxypentyl)amino]octan-2-ol [3](1.69 g, 4.69 mmol) at RT under inert atmosphere. Resultant reaction mixture was allowed to stir at RT for 16 h. Reaction progress was monitored by TLC/ELSD. Reaction mass evaporated under reduced pressure, then washed with heptane 5 times. Combined heptane fractions were evaporated under reduced pressure to give crude reaction mass. Crude was purified by prep HPLC (ACN/0.1% TFA in water) to give 1,5-bis({5-[bis(2-hydroxyoctyl)amino]pentyl}) 3-hydroxy-3-methylpentanedioate trifluoroacetic acid salt Compound XLIX (126 mg, 6.04% Yield) as a light yellow semi solid.

### Results:

1H-NMR (400 MHz, CDCl3)-δ 5.29 (s, 1H), 4.20-3.97 (m, 5H), 3.65-3.62 (m, 2H), 3.40-3.02 (m, 12H), 3.98-3.90 (m, 1H), 2.86-2.74 (m, 2H), 2.70-2.50 (m, 4H), 1.82-1.69 (m, 8H), 1.68-1.54 (m, 2H), 1.51-1.45 (m, 7H), 1.45-1.38 (m, 6H), 1.33-1.27 (brs, 30H), 0.89-0.86 (t, J=6.8 Hz, 12H) ppm.

ELSD analysis: Purity 99.91%, Calculated C₄₈H₉₆N₂O₉=844.71, Observed=845.55 (m/z, M+H+).

### Example 15: Synthesis of Compound L

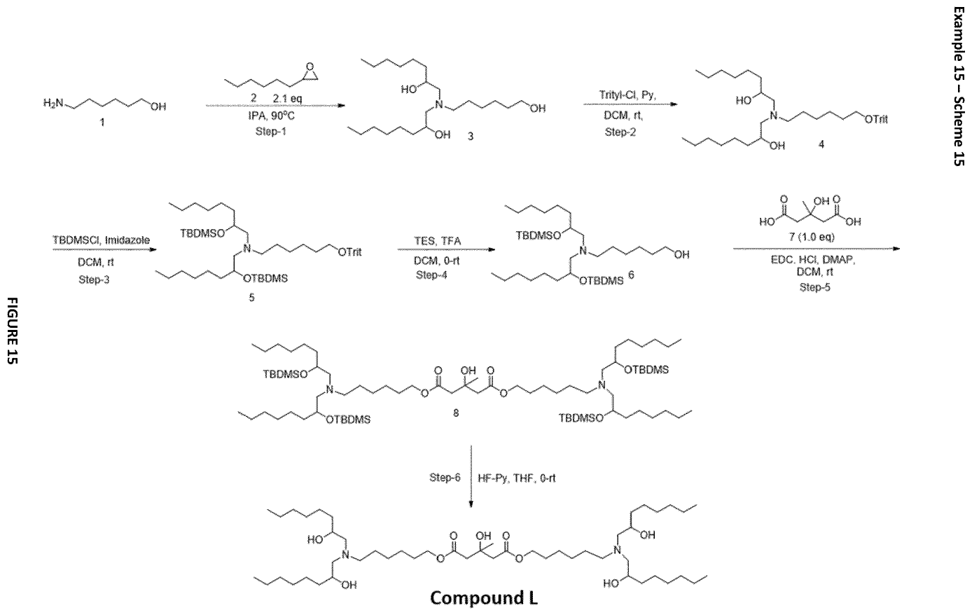

For example, the compounds of the invention may be prepared according to Scheme 15 (as depicted in FIG. 15).

### Synthesis of Intermediate [3]

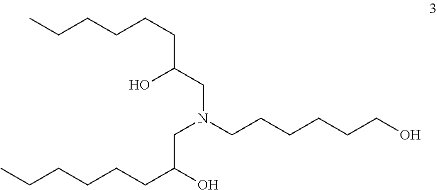 [SMILES: CCCCCCC(O)CN(CCCCCCO)CC(O)CCCCCC]

As depicted in Scheme 15: To a stirred solution of 6-aminohexan-1-ol [1](1.0 g, 8.53 mmol) in IPA (20 mL), added 2-hexyloxirane [2](2.41 g, 18.8 mmol). The reaction mixture was stirred for 16 h at 90° C. Reaction progress was monitored by TLC. After completion, reaction mixture was evaporated under reduced pressure to get crude, which was purified over silica gel flash column chromatography by using 0-5% methanol in dichloromethane gradient as eluent to get 1-[(6-hydroxyhexyl)(2-hydroxyoctyl)amino]octan-2-ol [3](2.0 g, 62.73% Yield:) as light green liquid.

### Result:

ELSD analysis: Purity 99.50%, Calculated C₂₂H₄₇NO₃=373.36, Observed=374.30 (m/z, M+H+).

### Synthesis of Intermediate [4]

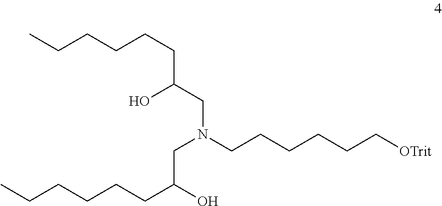

As depicted in Scheme 15: To a stirred solution of 1-[(6-hydroxyhexyl)(2-hydroxyoctyl)amino]octan-2-ol [3](2.0 g, 5.35 mmol) in DCM (25 mL), added pyridine (635 mg, 8.03 mmol) and (chlorodiphenylmethyl)benzene (1.81 g, 6.48 mmol) at RT. The reaction mixture was stirred for 16 h at RT. Reaction progress was monitored by TLC and ELSD data. After completion of reaction, reaction mass evaporated under reduced pressure to get crude 1-[(2-hydroxyoctyl)[6-(triphenylmethoxy)hexyl]amino]octan-2-ol [4](3.3 g, crude). Crude was used as such for next step.

### Result:

ELSD analysis: Purity 99.19%, Calculated C₄₁H₆₁NO₃=615.47, Observed=616.45 (m/z, M+H+).

### Synthesis of Intermediate [5]

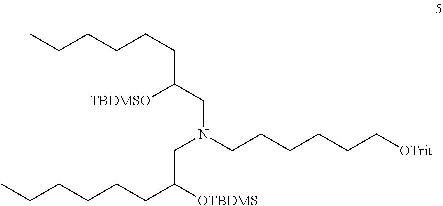

As depicted in Scheme 15: To a stirred solution of 1-[(2-hydroxyoctyl)[6-(triphenylmethoxy)hexyl]amino]octan-2-ol [4](3.3 g, 5.36 mmol) in DCM (50 mL), added 1H-imidazole (2.19 g, 32.1 mmol) and tert-butyl(chloro)dimethylsilane (3.23 g, 21.4 mmol) at room temperature under inert atmosphere. The reaction mixture was stirred for 16 h at RT. Reaction progress was monitored by TLC and ELSD data. After completion, reaction mass diluted with water (20 mL) and extracted with DCM (3×50 mL). Organic layers were combined and dried over sodium sulphate, filtered and evaporated under reduced pressure to get crude, which was purified over silica gel flash column chromatography (0-30% EtOAc in Heptane) to get 5,9-dihexyl-2,2,3,3,11,11,12,12-octamethyl-7-[6-(triphenylmethoxy)hexyl]-4,10-dioxa-7-aza-3,11-disilatridecane [5](4.0 g, 88.9% Yield after two step) as yellow viscous oil.

### Result:

ELSD analysis: Purity 99.34%, Calculated C₅₃H₈₉NO₃Si₂=843.64, Observed=844.50 (m/z, M+H+).

### Synthesis of Intermediate [6]

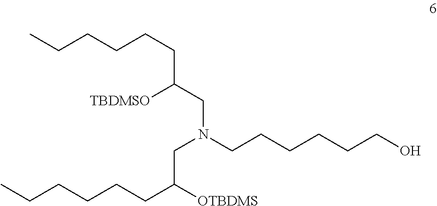 [SMILES: CCCCCCC(CN(CCCCCCO)CC(CCCCCC)O[Si](C)(C)C(C)(C)C)O[Si](C)(C)C(C)(C)C]

As depicted in Scheme 15: To stirred solution of 5,9-dihexyl-2,2,3,3,11,11,12,12-octamethyl-7-[6-(triphenylmethoxy)hexyl]-4,10-dioxa-7-aza-3,11-disilatridecane [5](4.0 g, 4.74 mmol) in dichloromethane (50 mL), simultaneously drop wise added trifluoroacetic acid (2.7 g, 23.7 mmol) and triethylsilyl (1.09 g, 9.47 mmol) at cooling condition. The reaction mixture was stirred for 3 h at RT. Reaction progress was monitor by TLC. After completion of reaction, reaction mass quenched by saturated sodium bicarbonate up to pH 8, and extracted with DCM (3×50 mL). The organic layers were combined, dried over sodium sulphate and evaporated under reduced pressure to get crude, which was purified on silica gel flash column chromatography by using 10-30% Ethyl acetate in Hexane to get 6-(5,9-dihexyl-2,2,3,3,11,11,12,12-octamethyl-4,10-dioxa-7-aza-3,11-disilatridecan-7-yl)hexan-1-ol (2.68 g, 94.1% Yield) as colorless liquid.

### Results:

ELSD analysis: Purity 85.17%, Calculated C₃₄H₇₅NO₃Si₂=601.53, Observed=698.40 (m/z, M+TFA+).

### Synthesis of Intermediate [8]

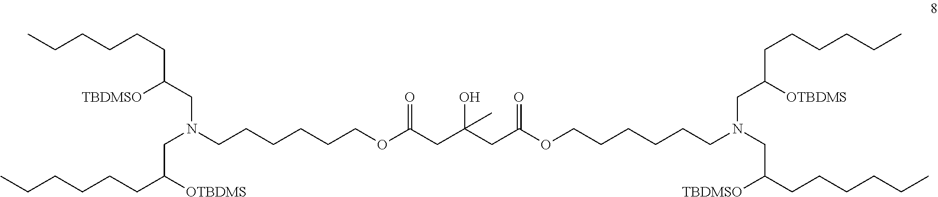 [SMILES: CCCCCCC(CN(CCCCCCOC(=O)CC(C)(O)CC(=O)OCCCCCCN(CC(CCCCCC)O[Si](C)(C)C(C)(C)C)CC(CCCCCC)O[Si](C)(C)C(C)(C)C)CC(CCCCCC)O[Si](C)(C)C(C)(C)C)O[Si](C)(C)C(C)(C)C]

As depicted in Scheme 15: To a stirred solution of 3-hydroxy-3-methylpentanedioic acid [7](250 mg, 1.54 mmol) in dichloromethane (15 mL), added 6-(5,9-dihexyl-2,2,3,3,11,11,12,12-octamethyl-4,10-dioxa-7-aza-3,11-disilatridecan-7-yl)hexan-1-ol [6](1.85 g, 3.07 mmol), {3-[cyano(ethyl)amino]propyl}dimethylazanium chloride (887 mg, 4.62 mmol) and 4-(dimethylamino)pyridin-1-ium (570 mg, 4.62 mmol) at room temperature under inert atmosphere. The reaction mixture was stirred for 48 h at RT. Reaction progress was monitored by TLC and ELSD. After completion, reaction mass was quenched by water (20 mL)) and extracted with DCM (3×50 mL). The organic layers were combined and dried over sodium sulphate, filtered and evaporated under reduced pressure to get crude, which was purified over silica gel flash column chromatography by using 0-30% ethyl acetate in n-hexane gradient as eluent to get 1,5-bis[6-(2,2,3,3,11,11,12,12-octamethyl-5,9-dioctyl-4,10-dioxa-7-aza-3,11-disilatridecan-7-yl)hexyl]3-hydroxy-3-methylpentanedioate [8](0.70 g, 35% Yield) as colorless liquid.

### Results:

ELSD analysis: Purity 99.80%, Calculated C₇₄H₁₅₆N₂O₉Si₄=1329.09, Observed=1330.90 (m/z, M+H+).

### Synthesis of Compound L

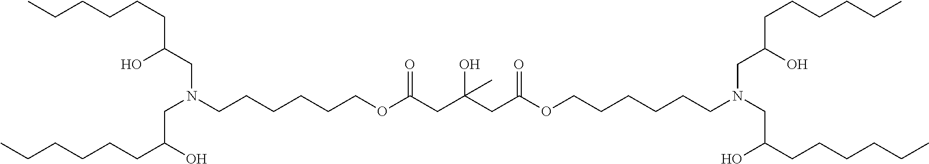 [SMILES: CCCCCCC(O)CN(CCCCCCOC(=O)CC(C)(O)CC(=O)OCCCCCCN(CC(O)CCCCCC)CC(O)CCCCCC)CC(O)CCCCCC]

As depicted in Scheme 15: To a stirred solution of 1,5-bis[6-(5,9-dihexyl-2,2,3,3,11,11,12,12-octamethyl-4,10-dioxa-7-aza-3,11-disilatridecan-7-yl)hexyl]3-hydroxy-3-methylpentanedioate [8](650 mg, 489 μmol) in tetrahydrofuran (10 mL), added pyridine hydrofluoride (484 mg, 4.89 mmol) drop wise at 0° C. Reaction mixture allowed to stir for 16 h at RT. Progress of reaction was monitor by TLC and ELSD. After completion of reaction, reaction mass quenched by saturated sodium bicarbonate up to pH 8, and extracted with ethyl acetate (3×25 mL). The organic layers were combine, dried over sodium sulphate, filtered and evaporated under reduce pressure to get crude, which was purify over silica (0-5% MeOH in DCM) to give 1,5-bis({6-[bis(2-hydroxyoctyl)amino]hexyl}) 3-hydroxy-3-methylpentanedioate Compound L (0.3 g, 70.3% Yield) as pale yellow liquid.

### Results:

1H-NMR (400 MHz, CDCl3)-δ 4.14 (brs, 1H), 4.10-4.07 (t, J=6.8 Hz, 4H), 3.67-3.62 (m, 4H), 2.70-2.53 (m, 8H), 2.47-2.36 (m, 8H), 1.67-1.60 (m, 4H), 1.51-1.25 (m, 55H), 0.89-0.86 (t, J=6.8 Hz, 12H) ppm.

ELSD analysis: Purity 99.06%, Calculated C₅₀H₁₀₀N₂O₉=872.74, Observed=873.6 (m/z, M+H+).

### Example 16: Synthesis of Compound LI

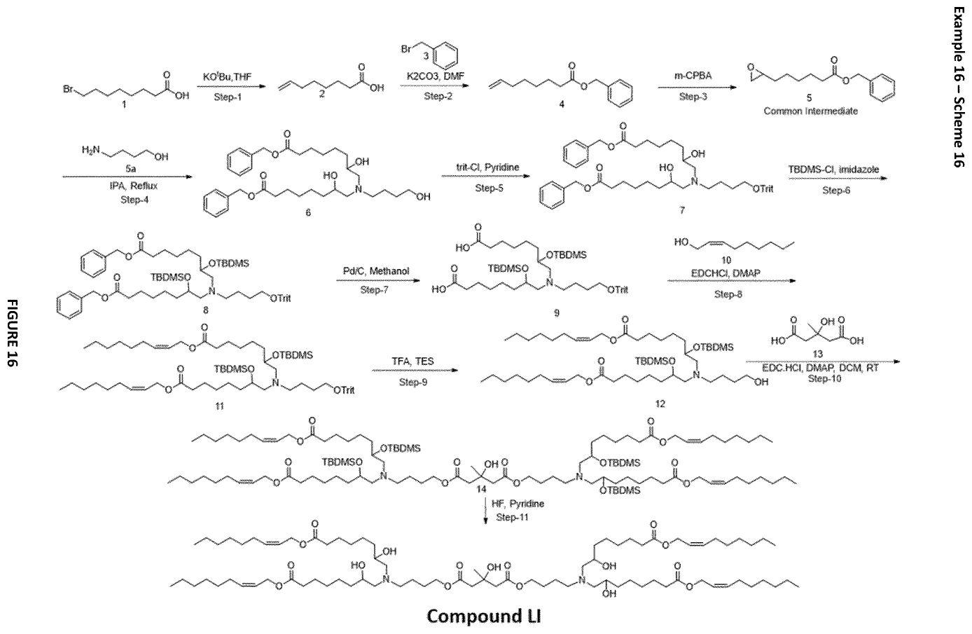

For example, the compounds of the invention may be prepared according to Scheme 16 (as depicted in FIG. 16).

### Synthesis of Intermediate [2]

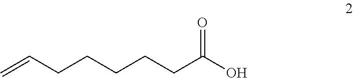

As depicted in Scheme 16: To a stirred solution of 8-bromooctanoic acid [1](100 g, 0.448 mol) in tetrahydrofuran (3.25 L, 39.9 mol), potassium 2-methylpropan-2-olate (226 g, 2.02 mol) was added at RT under nitrogen atmosphere. The reaction mixture was allowed to stir at 90° C. for 16 h. The progress of reaction was monitored by TLC. After completion, reaction mixture quenched by conc. 2M HCl up to pH 3 and extracted by ethyl acetate (3×250 mL), combined organic layer was dried over anhydrous sodium sulphate, filtered and evaporated under reduced pressure to get oct-7-enoic acid (61.0 g, 429 mmol) as a light yellow liquid.

### Result:

1H-NMR (400 MHz, CDCl3)-δ 11.96 (s, 1H), 5.83-5.73 (m, 1H), 5.01-4.92 (m, 2H), 2.20-2.16 (t, J=7.2 Hz, 2H), 2.03-1.98 (m, 2H), 1.54-1.45 (m, 2H), 1.38-1.30 (m, 2H), 1.30-1.23 (m, 2H).

### Synthesis of Intermediate [4]

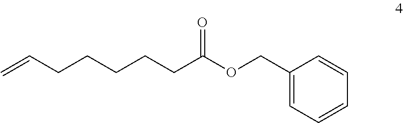 [SMILES: C=CCCCCCC(=O)OCc1ccccc1]

As depicted in Scheme 16: To a stirred solution of oct-7-enoic acid [2](61 g, 429 mmol) in dimethylformamide (610 mL, 7.88 mol) was added dipotassium carbonate (296 g, 2.14 mol) and (bromomethyl)benzene [3](80.7 g, 472 mmol). The reaction mixture was stirred for 16 h at RT. The progress of reaction was monitored by TLC. After completion of the reaction, RM was quench by ice cold water (1.5 Lit) and extracted by diethyl ether (3×500 mL). Combined organic layer dried over anhydrous sodium sulphate, filtered and reduced under vacuum. The crude was purified over silica gel flash column chromatography, by using 0-10% ethyl acetate in heptane gradient as eluent to give benzyl oct-7-enoate [4](80 g, 80.27% yield) as alight yellow liquid.

### Result:

1H-NMR (400 MHz, CDCl3)-7.39-7.26 (m, 5H), 5.84-5.74 (m, 1H), 5.11 (s, 2H), 5.01-4.92 (m, 2H), 2.38-2.34 (t, J=7.6 Hz, 2H), 2.06-2.01 (q, J=7.6 Hz, 2H), 1.69-1.61 (m, 2H), 1.44-1.27 (m, 4H) ppm.

### Synthesis of Intermediate [5]

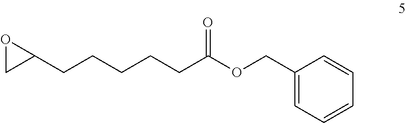 [SMILES: O=C(CCCCCC1CO1)OCc1ccccc1]

As depicted in Scheme 16: To a stirred solution of benzyl oct-7-enoate [4](80 g, 344 mmol) in DCM (941 mL), 3-chlorobenzene-1-carboperoxoic acid (89.1 g, 517 mmol) was added portion wise under nitrogen atmosphere. The reaction mixture was stirred for 16 h at RT. The progress of reaction was monitored by TLC. After completion, reaction was quench by saturated solution of sodium bicarbonate up to pH 8 and extracted by DCM (2×500 mL). The organic layer was dried over anhydrous sodium sulphate, filtered and reduced under vacuum. The crude was purified over silica gel flash column chromatography by using 5-15% gradient of ethyl acetate in heptane as eluent to give benzyl 6-(oxiran-2-yl)hexanoate [5] (61 g, 71% yield) as a colorless liquid.

### Result:

1H-NMR (400 MHz, CDCl3)-7.37-7.32 (m, 5H), 5.11 (s, 2H), 2.90-2.87 (m, 1H), 2.75-2.73 (m, 1H), 2.46-2.44 (m, 1H), 2.39-2.35 (t, J=7.6 Hz, 2H), 1.70-1.63 (m, 2H), 1.56-1.43 (m, 4H), 1.39-1.26 (m, 2H) ppm.

### Synthesis of Intermediate [6]

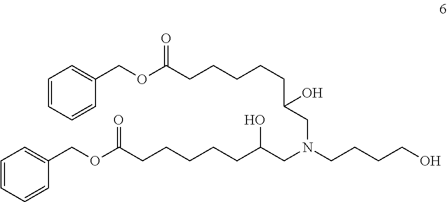 [SMILES: O=C(CCCCCC(O)CN(CCCCO)CC(O)CCCCCC(=O)OCc1ccccc1)OCc1ccccc1]

As depicted in Scheme 16: To the stirred solution of benzyl 6-(oxiran-2-yl)hexanoate (8.36 g, 33.7 mmol) in isopropanol (100 mL, 951 mmol), 4-aminobutan-1-ol [5a](1.2 g, 13.5 mmol) was added at RT. The reaction mixture was stirred for 16 h 90° C. Progress of reaction was monitor by TLC. Then solvent was evaporated to get crude compound. The crude compound was purified by column chromatography using silica gel and 0-40% ethyl acetate in heptane as eluent to afford benzyl 8-{[8-(benzyloxy)-2-hydroxy-8-oxooctyl](4-hydroxybutyl)amino}-7-hydroxy octanoate [6](6.7 g, 85% yield) as yellowish viscous oil.

### Result:

ELSD analysis: Purity 99.59%, Calculated C₃₄H₅₁NO₇=585.37, Observed=586.30 (m/z, M+H+).

### Synthesis of Intermediate [7]

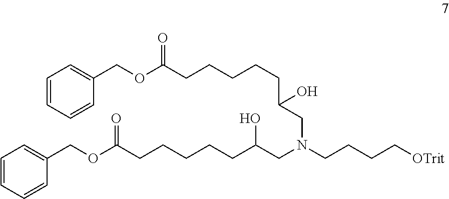

As depicted in Scheme 16: To a stirred solution of benzyl 8-{[8-(benzyloxy)-2-hydroxy-8-oxooctyl](4-hydroxybutyl)amino}-7-hydroxyoctanoate [6](6.7 g, 11.4 mmol) in dichloromethane (50 mL, 781 mmol), (chlorodiphenylmethyl)benzene (4.08 g, 14.6 mmol) and pyridine (1.58 g, 20 mmol) was added at 0° C., under inert atmosphere. The reaction mass allowed to stir at RT for 16 h. Progress of reaction was monitor by TLC/ELSD. Reaction mass was concentrated with repeated addition of DCM under reduced pressure, then dried under high vacuum to give benzyl 8-{[8-(benzyloxy)-2-hydroxy-8-oxooctyl][4-(triphenylmethoxy)butyl]amino}-7-hydroxyoctanoate [7](11 g, 99.76% yield) as colorless liquid. Compound was used as such for next step.

### Result:

ELSD analysis: Purity 99.94%, Calculated C₅₃H₆₅NO₇=827.48, Observed=828.30 (m/z, M+H+).

### Synthesis of Intermediate [8]

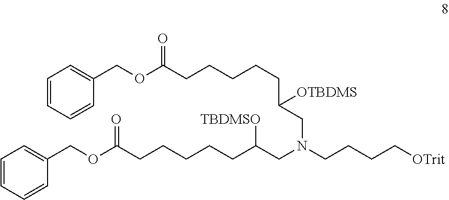

As depicted in Scheme 16: To a stirred solution of benzyl 8-{[8-(benzyloxy)-2-hydroxy-8-oxooctyl][4-(triphenylmethoxy)butyl]amino}-7-hydroxyoctanoate [7](11 g, 13.3 mmol) in dichloromethane (250 mL, 1.56 mol), 1H-imidazole (5.43 g, 79.7 mmol) and tert-butyl(chloro)dimethylsilane (8.01 g, 53.1 mmol) were added successively under inert atmosphere. The reaction mixture was stirred for 16 h at RT. Progress of reaction was monitor by TLC. Reaction mixture was diluted with DCM (100 mL) and washed with cold water (2×100 mL). Organic layer was dried over sodium sulphate, filtered and evaporated under reduced pressure to gives crude reaction mass. Crude was purified over silica gel flash column chromatography (30% EtOAc in Hexane) to afford benzyl 8-{9-[6-(benzyloxy)-6-oxohexyl]-11,11,12,12-tetramethyl-1,1,1-triphenyl-2,10-dioxa-7-aza-11-silatridecan-7-yl}-7-[(tert-butyldimethylsilyl)oxy]octanoate [8](11.5 g, 81.9% yield) as colourless liquid.

### Result:

ELSD analysis: Purity 99.93%, Calculated C₆₅H₉₃NO₇Si₂=1055.65, Observed=1056.50 (m/z, M+H+).

### Synthesis of Intermediate [9]

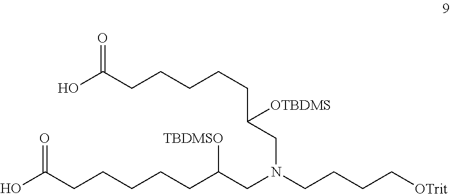

As depicted in Scheme 16: To a stirred solution of benzyl 8-{9-[6-(benzyloxy)-6-oxohexyl]-11,11,12,12-tetramethyl-1,1,1-triphenyl-2,10-dioxa-7-aza-11-silatridecan-7-yl}-7-[(tert-butyldimethylsilyl)oxy]octanoate [8](8 g, 7.57 mmol), in methanol (20 mL), tetrahydrofuran (20 mL), was added palladium on carbon (10% w/w with 50% moisture) (4.03 g, 37.9 mmol) portion wise under inert atmosphere. Reaction mixture was degassed with vacuum and allowed to stir at RT under hydrogen balloon pressure for 16 h. After, completion of reaction, reaction mixture was filtered through celite, washed two times with methanol. Methanol was evaporated to dryness under reduce pressure to get 7-[(tert-butyldimethylsilyl)oxy]-8-[9-(5-carboxypentyl)-11,11,12,12-tetramethyl-1,1,1-triphenyl-2,10-dioxa-7-aza-11-silatridecan-7-yl]octanoic acid [9](6.0 g, 90.4% yield) as colourless liquid.

### Result:

ELSD analysis: Purity 99.68%, Calculated C₅₁H₈₁NO₇Si₂=875.56, Observed=876.35 (m/z, M+H+).

### Synthesis of Intermediate [11]

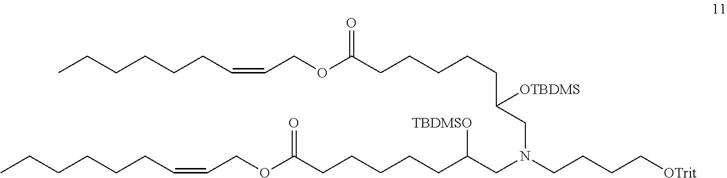

As depicted in Scheme 16: To a stirred solution of 7-[(tert-butyldimethylsilyl)oxy]-8-[9-(5-carboxypentyl)-11,11,12,12-tetramethyl-1,1,1-triphenyl-2,10-dioxa-7-aza-11-silatridecan-7-yl]octanoic acid [9](1.8 g, 2.05 mmol) in dichloromethane (50 mL), {3-[cyano(ethyl)amino]propyl}dimethylazanium chloride (1.18 g, 6.16 mmol) and 4-(dimethylamino)pyridin-1-ium (253 mg, 2.05 mmol) were added at RT under inert atmosphere. To the resulting reaction mixture (2Z)-non-2-en-1-ol (730 mg, 5.13 mmol) was added at RT under inert atmosphere. The reaction mixture was stirred for 16 h at RT. Progress of reaction was monitor by TLC. SM was consumed completely. Reaction mass was evaporated under reduced pressure. Crude was purified over silica gel flash column chromatography by using 10-30% EtOAc in Hexane gradient as eluent to give (2Z)-non-2-en-1-yl 7-[(tert-butyldimethylsilyl)oxy]-8-(11,11,12,12-tetramethyl-9-{6-[(2Z)-non-2-en-1-yloxy]-6-oxohexyl}-1,1,1-triphenyl-2,10-dioxa-7-aza-11-silatridecan-7-yl)octanoate [11](2.0 g, 86.57% yield) as pale yellow liquid.

### Result:

ELSD analysis: Purity 99.62%, Calculated C₆₉H₁₁₃NO₇Si₂=1123.81, Observed=1124.50 (m/z, M+H+).

### Synthesis of Intermediate [12]

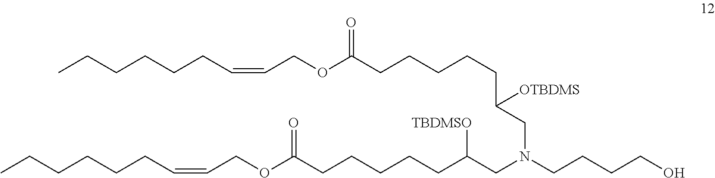 [SMILES: CCCCCC/C=C\COC(=O)CCCCCC(CN(CCCCO)CC(CCCCCC(=O)OC/C=C\CCCCCC)O[Si](C)(C)C(C)(C)C)O[Si](C)(C)C(C)(C)C]

As depicted in Scheme 16: To the stirred solution of (2Z)-non-2-en-1-yl 7-[(tert-butyldimethylsilyl)oxy]-8-(11,11,12,12-tetramethyl-9-{6-[(2Z)-non-2-en-1-yloxy]-6-oxohexyl}-1,1,1-triphenyl-2,10-dioxa-7-aza-11-silatridecan-7-yl)octanoate [11](1.8 g, 1.6 mmol) in dichloromethane (20 mL), triethylsilyl (369 mg, 3.2 mmol) and trifluoroacetic acid (912 mg, 8 mmol) added dropwise successively at 0° C. under inert atmosphere. The reaction was allowed to stir at RT for 4 h. Progress of reaction was monitor by TLC. The solvent was evaporated under reduced pressure, and residue was basified using saturated NaHCO₃ aq. solution up to pH 8. The compound was extracted with diethyl ether (2×50 mL). The combined organic layer dried over Na₂SO₄, filtered and evaporated to get crude (2Z)-non-2-en-1-yl 7-[(tert-butyldimethylsilyl)oxy]-8-({2-[(tert-butyldimethylsilyl)oxy]-8-[(2Z)-non-2-en-1-yloxy]-8-oxooctyl}(4-hydroxybutyl)amino) octanoate [12](1.4 g, crude) as colourless liquid compound. The crude was used as such for next step.

### Result:

ELSD analysis: Purity 99.56%, Calculated C₅₀H₉₉NO₇Si₂=881.70, Observed=882.50 (m/z, M+H+).

### Synthesis of Intermediate [14]

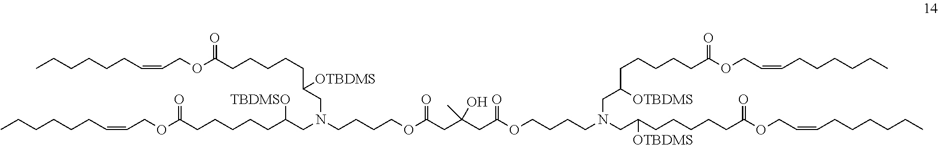 [SMILES: CCCCCC/C=C\COC(=O)CCCCCC(CN(CCCCOC(=O)CC(C)(O)CC(=O)OCCCCN(CC(CCCCCC(=O)OC/C=C\CCCCCC)O[Si](C)(C)C(C)(C)C)CC(CCCCCC(=O)OC/C=C\CCCCCC)O[Si](C)(C)C(C)(C)C)CC(CCCCCC(=O)OC/C=C\CCCCCC)O[Si](C)(C)C(C)(C)C)O[Si](C)(C)C(C)(C)C]

As depicted in Scheme 16: To a stirred solution of 3-hydroxy-3-methylpentanedioic acid [13](122 mg, 118 μmol) in dichloromethane (20 mL), {3-[cyano(ethyl)amino]propyl}dimethylazanium chloride (433 mg, 2.25 mmol) and 4-(dimethylamino)pyridin-1-ium (93 mg, 0.752 mmol) were added at RT under inert atmosphere. After 10 min (2Z)-non-2-en-1-yl 7-[(tert-butyldimethylsilyl)oxy]-8-({2-[(tert-butyldimethylsilyl)oxy]-8-[(2Z)-non-2-en-1-yloxy]-8-oxooctyl}(4-hydroxybutyl) amino)octanoate [12](218 mg, 247 μmol) was added to the resulting reaction mixture. The reaction mixture was stirred for 16 h at RT. After 16 h reaction progress was monitor by TLC, SM was consumed completely. Reaction mass was evaporated under reduced pressure to afford crude which was purified over silica gel flash column chromatography by using 0-40% ethyl acetate in heptane gradient as eluent to give 1,5-bis({4-[2,2,3,3,11,11,12,12-octamethyl-5,9-bis({6-[(2Z)-non-2-en-1-yloxy]-6-oxohexyl})-4,10-dioxa-7-aza-3,11-disilatridecan-7-yl]butyl}) 3-hydroxy-3-methylpentanedioate [14](0.55 g, 35% yield) as pale yellow liquid.

### Result:

ELSD analysis: Purity 98.45%, Calculated C₁₀₆H₂₀₄N₂O₁₇Si₄=1889.42, Observed=1890.15 (m/z, M+H+).

### Synthesis of Compound LI

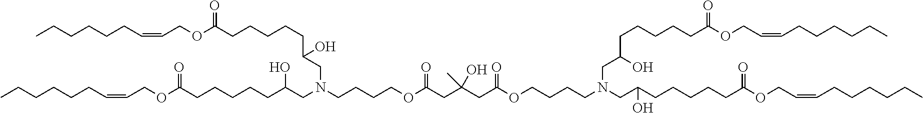 [SMILES: CCCCCC/C=C\COC(=O)CCCCCC(O)CN(CCCCOC(=O)CC(C)(O)CC(=O)OCCCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC]

As depicted in Scheme 16: To a stirred solution of 1,5-bis({4-[2,2,3,3,11,11,12,12-octamethyl-5,9-bis({6-[(2Z)-non-2-en-1-yloxy]-6-oxohexyl})-4,10-dioxa-7-aza-3,11-disilatridecan-7-yl]butyl}) 3-hydroxy-3-methylpentanedioate [14](0.5 g, 264 μmol) in dichloromethane (5 mL, 78.1 mmol), pyridine hydrofluoride (131 mg, 1.32 mmol) was added at 0° C. under inert atmosphere. The reaction mixture was stirred for 16 h at RT. The progress of reaction was monitored by TLC. After completion of reaction, reaction mixture was quenched by saturated sodium bicarbonate solution up to pH 8 and extracted with ethyl acetate (3×50 mL). The organic layers were combined, dried over anhydride sodium sulphate, filtered and concentrated under reduced pressure. The crude was purified by flash column chromatography (SiO2: 0-5% methanol in dichloromethane), to obtain 1,5-bis({4-[bis({2-hydroxy-8-[(2Z)-non-2-en-1-yloxy]-8-oxooctyl})amino]butyl}) 3-hydroxy-3-methylpentanedioate Compound LI (145 mg, 38% Yield) as colourless liquid.

### Result:

1H-NMR (400 MHz, CDCl3)-5.67-5.60 (m, 4H), 5.54-5.48 (m, 4H), 4.62-4.60 (d, J=6.8 Hz, 8H), 4.14-4.09 (m, 6H), 4.00-3.60 (brs, 4H), 2.72-2.68 (m, 2H), 2.62-2.58 (m, 2H), 2.33-2.29 (t, J=7.6 Hz, 8H), 2.12-2.07 (m, 8H), 1.80-1.60 (m, 16H), 1.56-1.20 (m, 69H), 0.90-0.86 (t, J=6.8 Hz, 12H) ppm.

ELSD analysis: Purity 97.96%, Calculated C₃₂H₁₄₃N₂O₁₇=1433.08, Observed=1433.75 (m/z, M+H+).

### Example 17: Synthesis of Compound LII

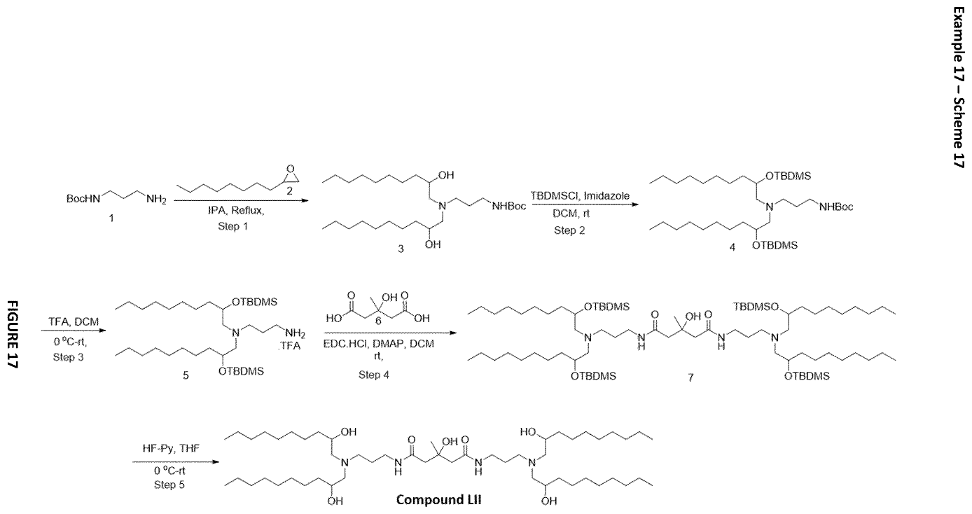

For example, the compounds of the invention may be prepared according to Scheme 17 (as depicted in FIG. 17).

### Synthesis of Intermediate [3]

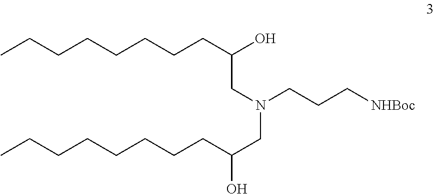 [SMILES: CCCCCCCCC(O)CN(CCCNC(=O)OC(C)(C)C)CC(O)CCCCCCCC]

As depicted in Scheme 17: To a stirred solution of tert-butyl (3-aminopropyl)carbamate [1](2.0 g, 11.5 mmol) and 2-octyloxirane [2](3.95 g, 25.3 mmol) in isopropanol (40 mL) was added ethylbis(propan-2-yl)amine (1.48 g, 11.5 mmol). The resultant reaction mixture was stirred for 16 h at 90° C. The reaction progress was monitored by TLC and ELSD. Starting material was consumed completely. Reaction mass was concentrated under reduced pressure to get crude compound, which was purified by silica gel flash column chromatography (0-5% MeOH in DCM) to give tert-butyl N-{3-[bis(2-hydroxydecyl)amino]propyl}carbamate [3](5.8 g, 93.4% yield) as colourless liquid Result:

ELSD analysis: Purity 96.6%, Calculated C₂₈H₅₈N₂O₄=486.44, Observed=487.40 (m/z, M+H+).

### Synthesis of Intermediate [4]

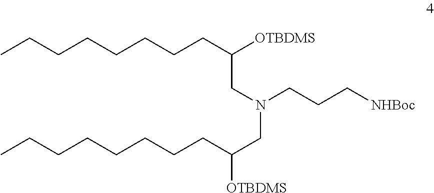 [SMILES: CCCCCCCCC(CN(CCCNC(=O)OC(C)(C)C)CC(CCCCCCCC)O[Si](C)(C)C(C)(C)C)O[Si](C)(C)C(C)(C)C]

As depicted in Scheme 17: To a solution of tert-butyl N-{3-[bis(2-hydroxydecyl)amino]propyl}carbamate [3](5.5 g, 11.3 mmol) in dichloromethane (100 mL), was added 1H-imidazole (12.3 g, 181 mmol), followed by tert-butyl(chloro)dimethylsilane (17 g, 113 mmol) at 0° C. under nitrogen atmosphere. The resulting mixture was stirred at room temperature for overnight. After 16 h, TLC (20% EtOAc in hexanes) analysis indicated completion of the reaction. The reaction mixture was diluted with dichloromethane (50 mL) and washed with water (2×100 mL) and brine solution (50 mL). The organic layer was dried over anhydrous Na₂SO₄, filtered and concentrated. The crude residue was purified over silica gel flash column chromatography (0-6% EtOAc in heptane) to obtain tert-butyl N-[3-(2,2,3,3,11,11,12,12-octamethyl-5,9-dioctyl-4,10-dioxa-7-aza-3,11-disilatridecan-7-yl)propyl]carbamate [4](8 g, 98% yield) as colourless liquid.

### Result:

ELSD analysis: Purity 99.84%, Calculated C₄₀H₈₆N₂O₄Si₂=714.61, Observed=715.50 (m/z, M+H+).

### Synthesis of Intermediate [5]

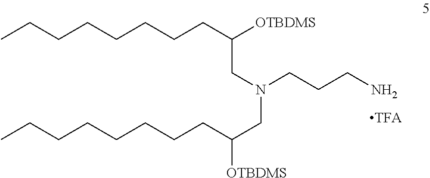 [SMILES: CCCCCCCCC(CN(CCCN)CC(CCCCCCCC)O[Si](C)(C)C(C)(C)C)O[Si](C)(C)C(C)(C)C]

As depicted in Scheme 17: To a stirred solution of tert-butyl N-[3-(2,2,3,3,11,11,12,12-octamethyl-5,9-dioctyl-4,10-dioxa-7-aza-3,11-disilatridecan-7-yl)propyl]carbamate [4](4.0 g, 5.59 mmol) in dichloromethane (20 mL), was added trifluoroacetic acid (6.42 mL, 83.9 mmol) at 0° C. The reaction mixture was stirred at RT for 6 h. Progress of reaction was monitored by TLC. Reaction mixture was concentrated under reduced pressure with repeated (2 times) addition of DCM and dried under high vacuum to give crude 7-(3-aminopropyl)-2,2,3,3,11,11,12,12-octamethyl-5,9-dioctyl-4,10-dioxa-7-aza-3,11-disilatridecane TFA salt [5](2.14 g, 61.9%) as colourless liquid.

### Result:

ELSD analysis: Purity 99.71%, Calculated C₃₅H₇₃N₂O₂Si₂=614.56, Observed=615.45 (m/z, M+H+).

### Synthesis of Intermediate [7]

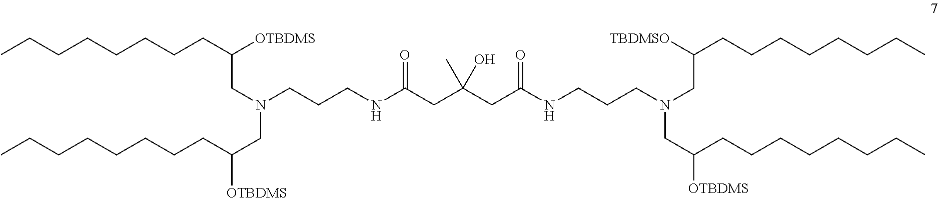 [SMILES: CCCCCCCCC(CN(CCCNC(=O)CC(C)(O)CC(=O)NCCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 17: To a solution of 7-(3-aminopropyl)-2,2,3,3,11,11,12,12-octamethyl-5,9-dioctyl-4,10-dioxa-7-aza-3,11-disilatridecane TFA salt [5](2.09 g, 3.39 mmol) in dichloromethane (20 mL) was added triethylamine (1.56 g, 15.4 mmol) at 0° C. After 5 min 4-(dimethylamino)pyridin-1-ium (380 mg, 3.08 mmol), 3-hydroxy-3-methylpentanedioic acid (250 mg, 1.54 mmol) and {3-[cyano(ethyl)amino]propyl}dimethylazanium chloride (739 mg, 3.85 mmol) was added to the resulting reaction mixture and stirred at RT. The resulting reaction mixture was stirred for 16 h at RT. Progress of reaction was monitored by TLC. The reaction mixture was diluted with dichloromethane (50 mL) and washed with water (20 mL) and brine solution (20 mL). The organic layer was dried over anhydrous Na₂SO₄, filtered and concentrated. The crude was purified over silica gel flash column (0-35% EtOAc in heptane) to give 3-hydroxy-3-methyl-N,N′-bis[3-(2,2,3,3,11,11,12,12-octamethyl-5,9-dioctyl-4,10-dioxa-7-aza-3,11-disilatridecan-7-yl)propyl]pentanediamide [7](0.7 g, 33.34 yield) as pale yellow liquid.

### Result:

ELSD analysis: Purity 99.78%, Calculated C₇₆H₁₆₂N₄O₇Si₄=1355.15, Observed=1355.80 (m/z, M+H+).

### Synthesis of Compound LII

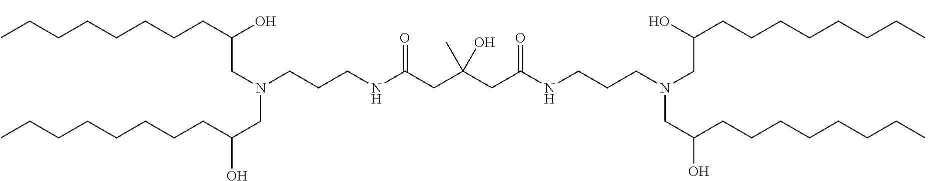 [SMILES: CCCCCCCCC(O)CN(CCCNC(=O)CC(C)(O)CC(=O)NCCCN(CC(O)CCCCCCCC)CC(O)CCCCCCCC)CC(O)CCCCCCCC]

As depicted in Scheme 17: To a stirred solution of 3-hydroxy-3-methyl-N,N′-bis[3-(2,2,3,3,11,11,12,12-octamethyl-5,9-dioctyl-4,10-dioxa-7-aza-3,11-disilatridecan-7-yl)propyl]pentanediamide [7](0.7 g, 516 μmol) in tetrahydrofuran (7 mL) was added pyridine hydrofluoride 70% (0.8 mL) at 0° C. Reaction mixture was allowed to stir for 16 h at RT. Reaction progress was monitor by TLC. After SM consumed, reaction mixture was quenched with aqueous sodium bicarbonate solution up to pH 8, and extracted with Ethyl acetate (2×50 mL). Organic layer was dried over anhy. sodium sulphate, filtered and concentrated under reduced pressure to get crude, which was dissolved in heptane (20 mL) wash with ACN (2×5 mL). The heptane layers was collected, dried under reduced pressure up to 5 mL remaining. This remaining solution was filtered by 0.22 μm filter, the filtrate was concentrated under reduced pressure to obtain N,N′-bis({3-[bis(2-hydroxydecyl)amino]propyl})-3-hydroxy-3-methylpentanediamide Compound LII (280 mg, 60.3% yield) as pale yellow liquid.

### Result:

1H NMR (400 MHz, CDCl3): δ 7.73-7.72 (m, 2H), 6.15-6.06 (m, 1H), 3.73-3.68 (m, 4H), 3.58-3.53 (m, 1H), 3.36-3.35 (m, 2H), 3.16-3.13 (m, 1H), 2.70-2.63 (m, 3H), 2.53-2.36 (m, 11H), 2.25-2.22 (m, 2H), 1.73-1.72 (m, 1H), 1.65-1.63 (m, 3H), 1.42-1.27 (m, 63H), 0.89-0.86 (t, J=6.8 Hz, 12H) ppm.

ELSD analysis: Purity 99.97%, Calculated C₅₂H₁₀₆N₄O₇=898.81, Observed=899.65 (m/z, M+H+).

### Example 18: Synthesis of Compound LIV

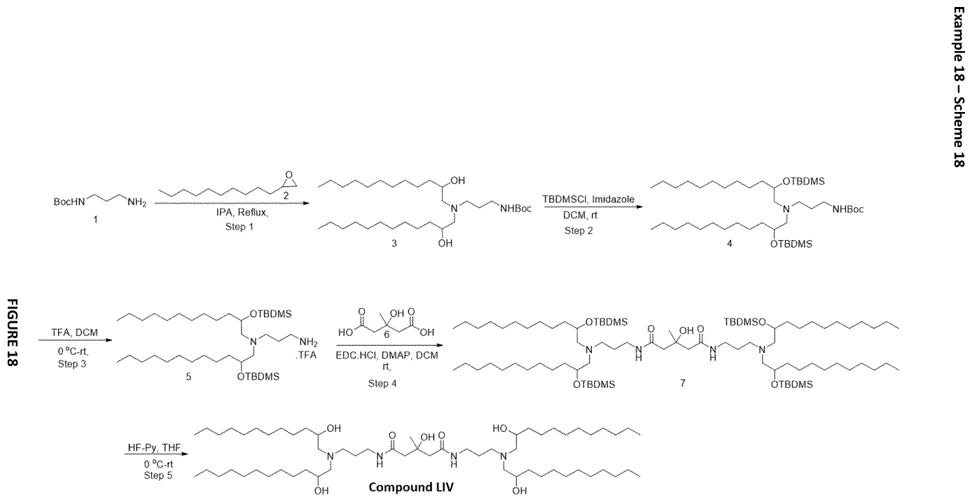

For example, the compounds of the invention may be prepared according to Scheme 18 (as depicted in FIG. 18).

### Synthesis of Intermediate [3]

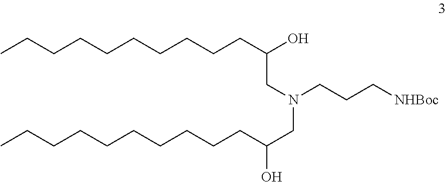 [SMILES: CCCCCCCCCCC(O)CN(CCCNC(=O)OC(C)(C)C)CC(O)CCCCCCCCCC]

As depicted in Scheme 18: To a stirred solution of tert-butyl (3-aminopropyl)carbamate [1](2.0 g, 11.5 mmol) and 2-decyloxirane [2](4.65 g, 25.3 mmol), in isopropanol (40 mL), ethylbis(propan-2-yl)amine (4 mL, 23 mmol) was added. The resultant reaction mixture was stirred for 16 h at 90° C. The reaction progress was monitored by TLC and ELSD. Starting material was consumed completely. Reaction mass was concentrated under reduced pressure get crude, which was purified by silica gel flash column chromatography (0-5% MeOH in DCM) to give tert-butyl N-{3-[bis(2-hydroxydodecyl)amino]propyl}carbamate [3](6.2 g, 95.6%) as colourless liquid Result:

ELSD analysis: Purity 96.47%, Calculated C₃₂H₆₆N₂O₄=542.50, Observed=543.45 (m/z, M+H+).

### Synthesis of Intermediate [4]

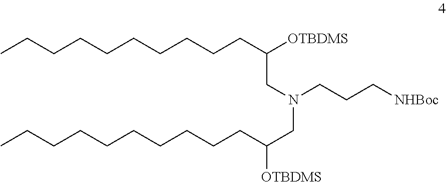 [SMILES: CCCCCCCCCCC(CN(CCCNC(=O)OC(C)(C)C)CC(CCCCCCCCCC)O[Si](C)(C)C(C)(C)C)O[Si](C)(C)C(C)(C)C]

As depicted in Scheme 18: To a solution of tert-butyl N-{3-[bis(2-hydroxydodecyl)amino]propyl}carbamate [3](6 g, 11.1 mmol), in dichloromethane (100 mL), was added 1H-imidazole (12 g, 177 mmol) followed by tert-butyl(chloro)dimethylsilane (16.7 g, 111 mmol) at 0° C. under nitrogen atmosphere. The resulting mixture was stirred at room temperature for overnight. Progress of reaction was monitor by TLC. The reaction mixture was diluted with dichloromethane (50 mL) and washed with water (2×50.0 mL) and brine solution (50.0 mL). The organic layer was dried over anhydrous Na₂SO₄, filtered and concentrated. The crude residue was purified over silica gel flash column chromatography (0-5% EtOAc in heptane) to obtain tert-butyl N-{3-[5,9-bis(decyl)-2,2,3,3,11,11,12,12-octamethyl-4,10-dioxa-7-aza-3,11-disilatridecan-7-yl]propyl}carbamate [4](7 g, 82% yield) as colourless liquid.

### Result:

ELSD analysis: Purity 99.94%, Calculated C₄₄H₉₄N₂O₄Si₂=770.68, Observed=771.50 (m/z, M+H+).

### Synthesis of Intermediate [5]

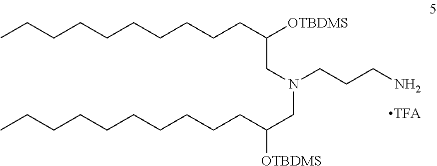 [SMILES: CCCCCCCCCCC(CN(CCCN)CC(CCCCCCCCCC)O[Si](C)(C)C(C)(C)C)O[Si](C)(C)C(C)(C)C]

As depicted in Scheme 18: To a stirred solution of tert-butyl N-{3-[5,9-bis(decyl)-2,2,3,3,11,11,12,12-octamethyl-4,10-dioxa-7-aza-3,11-disilatridecan-7-yl]propyl}carbamate (4.31 g, 5.59 mmol) in dichloromethane (25 mL), was added trifluoroacetic acid (6.42 mL, 83.9 mmol) at 0° C., then allowed to stir for 6 h. Progress of reaction was monitored by TLC. Reaction mixture was concentrated under reduced pressure with repeated (2 times) addition of DCM and dried under high vacuum to give crude 7-(3-aminopropyl)-5,9-bis(decyl)-2,2,3,3,11,11,12,12-octamethyl-4,10-dioxa-7-aza-3,11-disilatridecane TFA salt [5](2.3 g, 3.43 mmol) as colourless liquid.

### Result:

ELSD analysis: Purity 99.22%, Calculated C₃₉H₈₆N₂O₂Si₂=670.62, Observed=671.55 (m/z, M+H+).

### Synthesis of Intermediate [7]

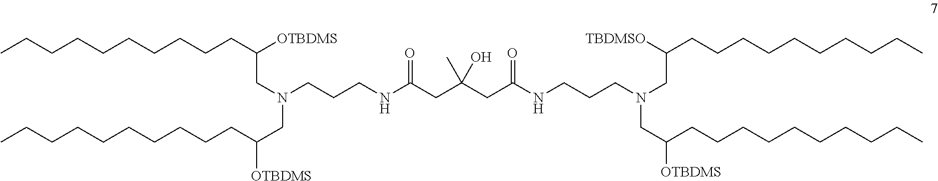 [SMILES: CCCCCCCCCCC(CN(CCCNC(=O)CC(C)(O)CC(=O)NCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 18: To a solution of 7-(3-aminopropyl)-5,9-bis(decyl)-2,2,3,3,11,11,12,12-octamethyl-4,10-dioxa-7-aza-3,11-disilatridecane TFA salt [5](2.28 g, 3.39 mmol) in dichloromethane (15 mL) was added triethylamine (1.56 g, 15.4 mmol) at 0° C. After 5 min, N,N-dimethylpyridin-4-amine (377 mg, 3.08 mmol), 3-hydroxy-3-methylpentanedioic acid [6](250 mg, 1.54 mmol) and {3-[cyano(ethyl)amino]propyl}dimethylazanium chloride (739 mg, 3.85 mmol) was added to the resulting reaction mixture and stirred at RT. The resulting reaction mixture was stirred for overnight at RT. Progress of reaction was monitor by TLC. The reaction mixture was diluted with dichloromethane (50 mL) and washed with water (20 mL) and brine solution (20 mL). The organic layer was dried over anhydrous Na₂SO₄, filtered and concentrated. The crude was purified over silica gel flash column (0-35% EtOAc in heptane) to give N-{3-[5,9-bis(decyl)-2,2,3,3,11,11,12,12-octamethyl-4,10-dioxa-7-aza-3,11-disilatridecan-7-yl]propyl}-N′-[3-(5-decyl-2,2,3,3,11,11,12,12-octamethyl-9-nonyl-4,10-dioxa-7-aza-3,11-disilatridecan-7-yl)propyl]-3-hydroxy-3-methylpentanediamide [7](0.5 g, 22% yield) as pale yellow liquid. ELSD analysis: Purity 99.39%, Calculated C₃₄H₁₇₃N₄O₇Si₄=1467.28, Observed=1468.95 (m/z, M+H+).

### Synthesis of Compound LIV

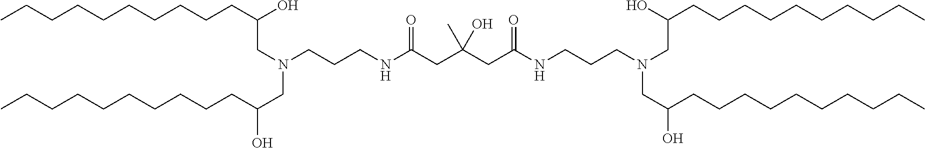 [SMILES: CCCCCCCCCCC(O)CN(CCCNC(=O)CC(C)(O)CC(=O)NCCCN(CC(O)CCCCCCCCCC)CC(O)CCCCCCCCCC)CC(O)CCCCCCCCCC]

As depicted in Scheme 18: To a solution of N-{3-[5,9-bis(decyl)-2,2,3,3,11,11,12,12-octamethyl-4,10-dioxa-7-aza-3,11-disilatridecan-7-yl]propyl}-N′-[3-(5-decyl-2,2,3,3,11,11,12,12-octamethyl-9-nonyl-4,10-dioxa-7-aza-3,11-disilatridecan-7-yl)propyl]-3-hydroxy-3-methylpentanediamide [7](0.5 g, 340 μmol) in tetrahydrofuran (5 mL, 61.4 mmol), was added pyridine hydrofluoride (1.07 mL, 11.9 mmol) at 0° C. Reaction mixture was allowed to stir for overnight at RT. Reaction progress was monitored by TLC. After SM was consumed, reaction mixture was quenched with cold aqueous sodium bicarbonate solution up to pH 8 and extracted with Ethyl acetate (2×50 mL). Organic layer was dried over anhydrous sodium sulphate, filtered and concentrated under reduced pressure to get crude, which was dissolved in heptane (20 mL) wash with ACN (2×5 mL). The heptane layers were collect dried under reduced pressure up to 5 mL remain, this remaining solution was filtered by 0.22 μm filter, the filtrate was concentrated under reduced pressure to obtain N,N′-bis({3-[bis(2-hydroxydodecyl)amino]propyl})-3-hydroxy-3-methylpentanediamide Compound LIV (230 mg, 66% yield) as colourless liquid.

### Result:

1H NMR (400 MHz, CDCl3): δ 7.79-7.68 (m, 2H), 6.16-6.05 (m, 1H), 3.68 (brs, 4H), 3.60-3.56 (m, 1H), 3.36 (br, 2H), 3.15-3.13 (m, 1H), 2.70-2.67 (m, 3H), 2.54-2.36 (m, 11H), 2.25-2.22 (m, 2H), 1.73-1.63 (m, 4H), 1.43-1.26 (m, 75H), 0.89-0.86 (t, J=6.8 Hz, 12H) ppm.

ELSD analysis: Purity 99.66%, Calculated C₆₀H₁₂₂N₄O₇=1010.93, Observed=1011.75 (m/z, M+H+).

### Example 19: Synthesis of Compound CIV

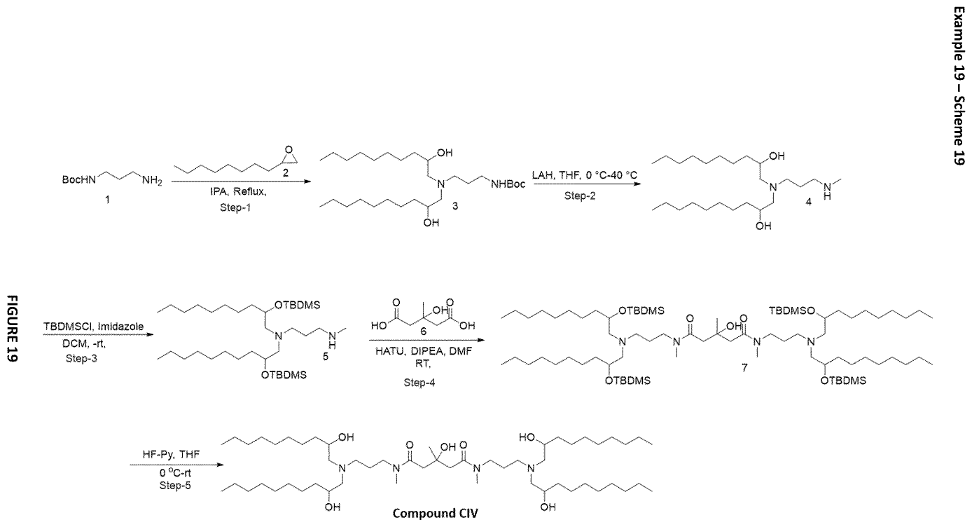

For example, the compounds of the invention may be prepared according to Scheme 19 (as depicted in FIG. 19).

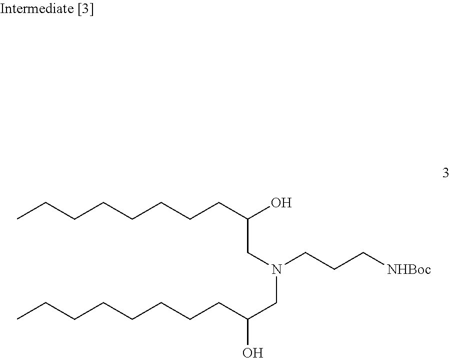 [SMILES: CCCCCCCCC(O)CN(CCCNC(=O)OC(C)(C)C)CC(O)CCCCCCCC]

As depicted in Scheme 19: To a stirred solution of tert-butyl (3-aminopropyl)carbamate [1](2.0 g, 11.5 mmol) and 2-octyloxirane [2](3.95 g, 25.3 mmol) in isopropanol (40 mL) was added ethylbis(propan-2-yl)amine (1.48 g, 11.5 mmol). The resultant reaction mixture was stirred for 16 h at 90° C. The reaction progress was monitored by TLC and ELSD. Starting material was consumed completely. Reaction mass was concentrated under reduced pressure to get crude compound, which was purified by silica gel flash column chromatography (0-5% MeOH in DCM) to give tert-butyl N-{3-[bis(2-hydroxydecyl)amino]propyl}carbamate [3](5.8 g, 93.4% yield) as a colourless liquid Result:

ELSD analysis: Purity 96.6%, Calculated C₂₈H₅₈N₂O₄=486.44, Observed=487.40 (m/z, M+H⁺).

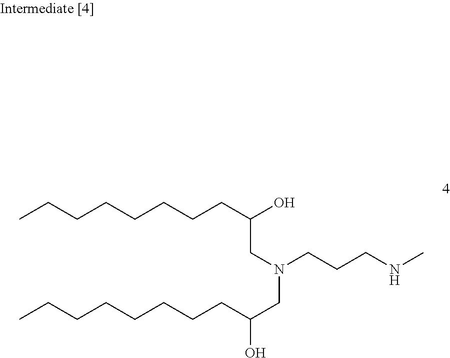 [SMILES: CCCCCCCCC(O)CN(CCCNC)CC(O)CCCCCCCC]

As depicted in Scheme 19: To a stirred solution of 3-[bis(2-hydroxydecyl)amino]propylamino-tert-butylformylate [3](3.4 g, 6.98 mmol) in tetrahydrofuran (34 mL), was added 2M solution of lithium aluminium hydride (530 mg, 14 mmol) in THF at 0° C., allow this reaction mixture for stirring at 40° C. for 48 h. Reaction progress was checked by TLC/ELSD after completion of reaction, reaction mixture was quenched by cold water. Fisher workup method was used for extraction with ethyl acetate, filter by sintered funnel. The organic layers were combined and evaporated under reduced pressure to obtain crude, used for purification by flash column chromatography (SiO2: 0-10 Methanol in DCM) to obtain 1-{(2-hydroxydecyl)[3-(methylamino)propyl]amino}-2-decanol [4](2.7 g, 96.47% Yield) as colourless liquid.

### Result:

ELSD analysis: Purity 99.06%, Calculated C₂₄H₅₂N₂O₂=400.40, Observed=401.40 (m/z, M+H⁺).

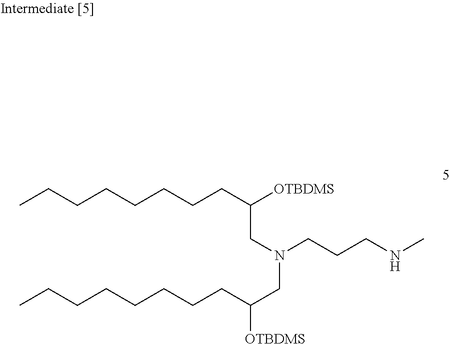 [SMILES: CCCCCCCCC(CN(CCCNC)CC(CCCCCCCC)O[Si](C)(C)C(C)(C)C)O[Si](C)(C)C(C)(C)C]

As depicted in Scheme 19: To a stirred solution of 1-{(2-hydroxydecyl)[3-(methylamino)propyl]amino}-2-decanol [4](2.7 g, 6.74 mmol) in dichloromethane (150 mL) were added imidazole (3.67 g, 53.9 mmol) and (tert-butyl)(chloro)bis(methyl)silane (6.09 g, 40.4 mmol) at room temperature for 16 hr. The progress of reaction was monitored by TLC. After completion, reaction mass was diluted with water and extract by DCM (2×80 mL). The organic layers were collect, dried over anhydrous sodium sulphate, concentrated under reduced pressure to get crude. The crude was purified by flash column chromatography (SiO₂: 0-20% ethyl acetate in hexane) to give desired product 1-(bis{2-[(tert-butyl)bis(methyl)siloxy]decyl}amino)-3-(methylamino)propane [5](2.2 g, 3.5 mmol) reddish liquid.

### Result:

ELSD analysis: Purity 99.99%, Calculated C₃₆H₃₀N₂O₂Si₂=628.58, Observed=629.45 (m/z, M+H⁺).

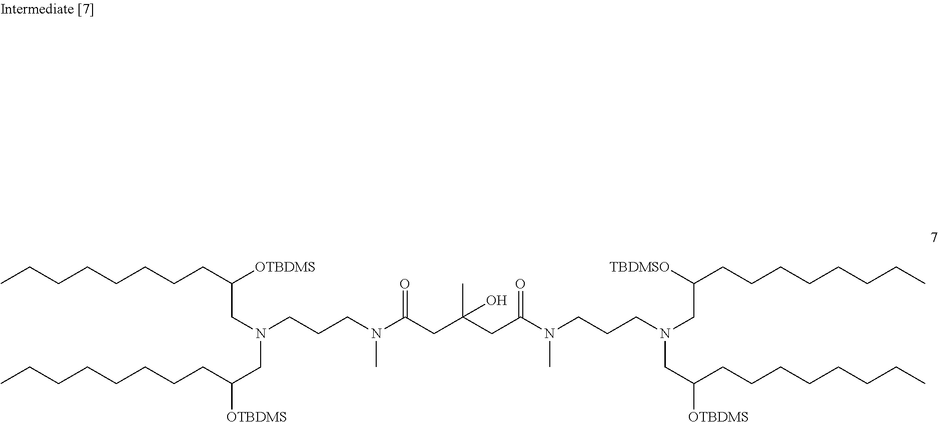 [SMILES: CCCCCCCCC(CN(CCCN(C)C(=O)CC(C)(O)CC(=O)N(C)CCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 19: To a stirred solution of 3-hydroxy-3-methylglutaric acid (260 mg, 1.6 mmol) in dimethylformamide (25 mL) were added N-ethylbis(isopropyl)amine (1.04 g, 8.02 mmol) and HATU reagent (1.52 g, 4.0 mmol) at RT for 15 min. After this 1-(bis{2-[(tert-butyl)bis(methyl)siloxy]decyl}amino)-3-(methylamino)propane (2.12 g, 3.37 mmol) and allow to stirred reaction at RT for 16 h. The progress of reaction was monitored by TLC and ELSD data. After completion, reaction mass quenched by water (7 mL) and extracted with ethyl acetate (3×25 mL), organic layer was dried over sodium sulphate and evaporate under reduced pressure to get crude and crude was purified by flash column chromatography (SiO2: 0-30% ethyl acetate in hexane) to give desired product as N′,N₅-bis(3-(bis(2-((tert-butyldimethylsilyl)oxy)decyl)amino)propyl)-3-hydroxy-N¹,N⁵,3-trimethylpentanediamide [7](750 mg, 33.78% Yield) as colourless liquid.

### Result:

ELSD analysis: Purity 99.49%, Calculated C₇₃H₁₆₆N₄O₇Si₄=1383.18, Observed=1384.85 (m/z, M+H⁺).

### Synthesis of Compound CIV

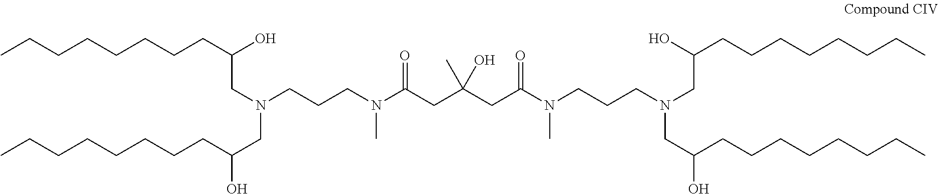 [SMILES: CCCCCCCCC(O)CN(CCCN(C)C(=O)CC(C)(O)CC(=O)N(C)CCCN(CC(O)CCCCCCCC)CC(O)CCCCCCCC)CC(O)CCCCCCCC]

As depicted in Scheme 19: To a stirred a solution of N¹,N⁵-bis(3-(bis(2-((tert-butyldimethylsilyl)oxy)decyl)amino) propyl)-3-hydroxy-N¹,N⁵,3-trimethylpentanediamide [7](0.7 g, 506 μmol) in tetrahydrofuran (7 mL) added hydrogen fluoride pyridine (1.37 mL 15.2 mmol) at cooling condition drop wise. Allow to stirred reaction mixture for 16 hr at RT, reaction progress was monitored by TLC and ELSD data. After completion, reaction mass quenched by saturated sodium bicarbonate and extracted with ethyl acetate (3×25 mL). The organic layer was combined, dried over sodium sulphate, evaporate under reduced pressure to get crude and crude was purified by flash column chromatography (SiO₂: 0-10% MeOH in DCM) to obtain N¹,N⁵-bis(3-(bis(2-hydroxydecyl)amino)propyl)-3-hydroxy-N¹,N⁵,3-trimethylpentanediamide [Compound CIV](340 mg, 72.51% Yield) as colourless liquid.

### Result:

¹H NMR (400 MHz, CDCl3): δ 4.20-3.88 (m, 4H), 3.61-3.30 (m, 8H), 3.20-3.06 (m, 12H), 3.12-2.81 (m, 12H), 2.76-2.53 (m, 4H), 2.20-1.90 (m, 4H), 1.58-1.38 (m, 8H), 1.35-1.20 (m, 46H), 0.89-0.86 (t, J=6.8 Hz, 12H).

ELSD analysis: Purity 99.86%, Calculated C₅₄H₁₁₀N₄O₇=926.84, Observed=927.55 (m/z, M+H+).

### Example 20: Synthesis of Compound LXXXIX

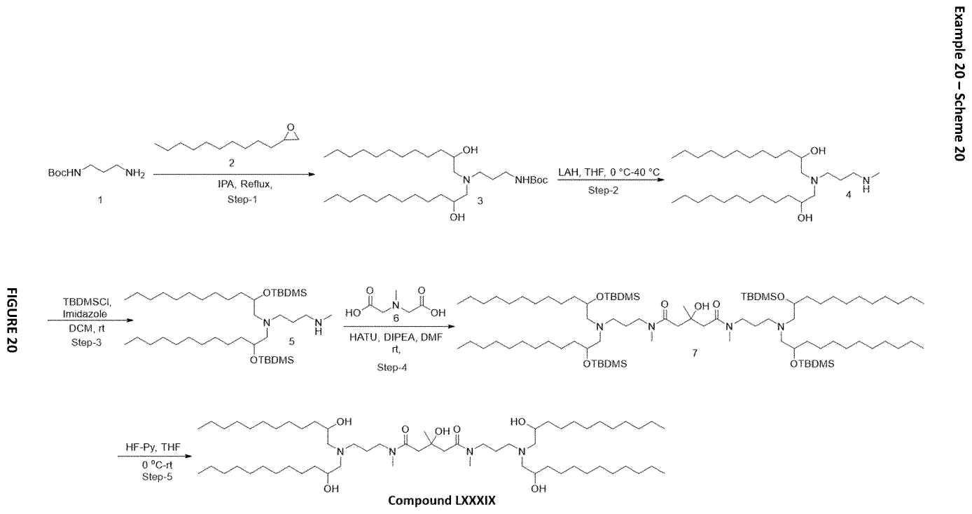

For example, the compounds of the invention may be prepared according to Scheme 20 (as depicted in FIG. 20).

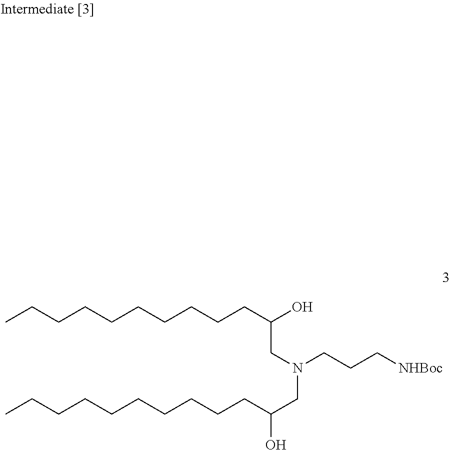 [SMILES: CCCCCCCCCCC(O)CN(CCCNC(=O)OC(C)(C)C)CC(O)CCCCCCCCCC]

As depicted in Scheme 20: To a stirred solution of tert-butyl (3-aminopropyl)carbamate [1](2.0 g, 11.5 mmol) and 2-decyloxirane [2](4.65 g, 25.3 mmol) in isopropanol (40 mL) was added ethylbis(propan-2-yl)amine (4 mL, 23 mmol). The resultant reaction mixture was stirred for 16 h at 90° C. The reaction progress was monitored by TLC and ELSD. Starting material was consumed completely. Reaction mass was concentrated under reduced pressure to give crude compound, which was purified by silica gel flash column chromatography (0-5% MeOH in DCM) to give tert-butyl N-{3-[bis(2-hydroxydodecyl)amino]propyl}carbamate [3](6.2 g, 95.6%) as a colourless liquid.

### Result:

ELSD analysis: Purity 96.47%, Calculated C₃₂H₆₆N₂O₄=542.50, Observed=543.45 (m/z, M+H⁺).

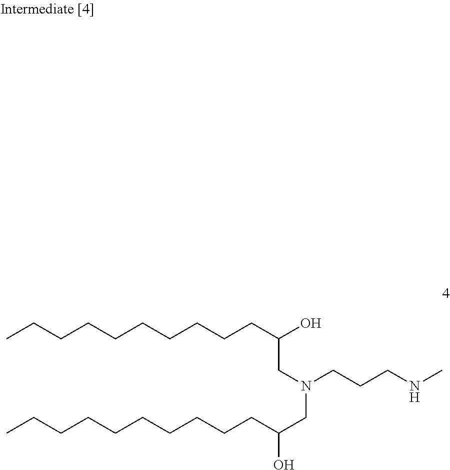 [SMILES: CCCCCCCCCCC(O)CN(CCCNC)CC(O)CCCCCCCCCC]

As depicted in Scheme 20: To a stirred solution of tert-butyl (3-(bis(2-hydroxydodecyl)amino)propyl)carbamate [3](6.4 g, 11.8 mmol) in tetrahydrofuran (50 mL), was added 2M solution of lithium aluminium hydride (895 mg, 23.6 mmol) in THF at 0° C., allow this reaction mixture for stirring at 40° C. for 48 h. Reaction progress was checked by TLC/ELSD after completion of reaction, reaction mixture was quenched by cold water. Fisher workup method was used for extraction with ethyl acetate, filter by sintered funnel. The organic layers were combined and evaporated under reduced pressure to obtain crude, used for purification by flash column chromatography (SiO2: 0-10 Methanol in DCM) to obtain 1,1′-((3-(methylamino)propyl)azanediyl)bis(dodecan-2-ol) [4](5.2 g, 96.56% Yield) as colourless liquid.

### Result:

ELSD analysis: Purity 99.70%, Calculated C₂₈H₆₀N₂O₂=456.47, Observed=457.40 (m/z, M+H⁺).

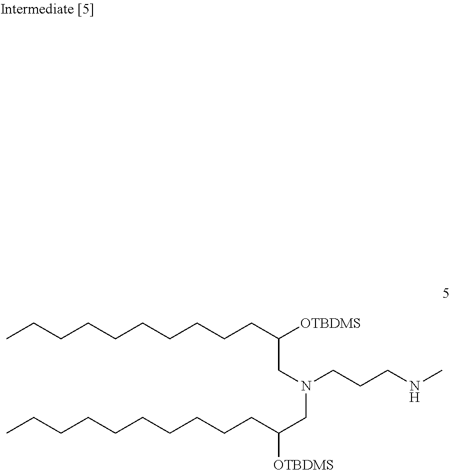 [SMILES: CCCCCCCCCCC(CN(CCCNC)CC(CCCCCCCCCC)O[Si](C)(C)C(C)(C)C)O[Si](C)(C)C(C)(C)C]

As depicted in Scheme 20: To a stirred solution of 1,1′-((3-(methylamino)propyl)azanediyl)bis(dodecan-2-ol) [4](3.5 g, 7.66 mmol) in dichloromethane (150 mL) were added imidazole (4.17 g, 61.3 mmol) and (tert-butyl)(chloro)bis(methyl)silane (6.93 g, 46.0 mmol) at room temperature for 16 hr. The progress of reaction was monitored by TLC. After completion, reaction mass was diluted with water and extract by DCM (2×100 mL). The organic layers were collect, dried over anhydrous sodium sulphate, concentrated under reduced pressure to get crude. The crude was purified by flash column chromatography (SiO₂: 0-20% ethyl acetate in hexane) to obtain N¹,N¹-bis(2-((tert-butyldimethylsilyl)oxy)dodecyl)-N³-methylpropane-1,3-diamine [5](3.27 g 62.27% Yield) as reddish liquid.

### Result:

ELSD analysis: Purity 99.72%, Calculated C₄₀H₃₃N₂O₂Si₂=684.64, Observed=685.55 (m/z, M+H⁺).

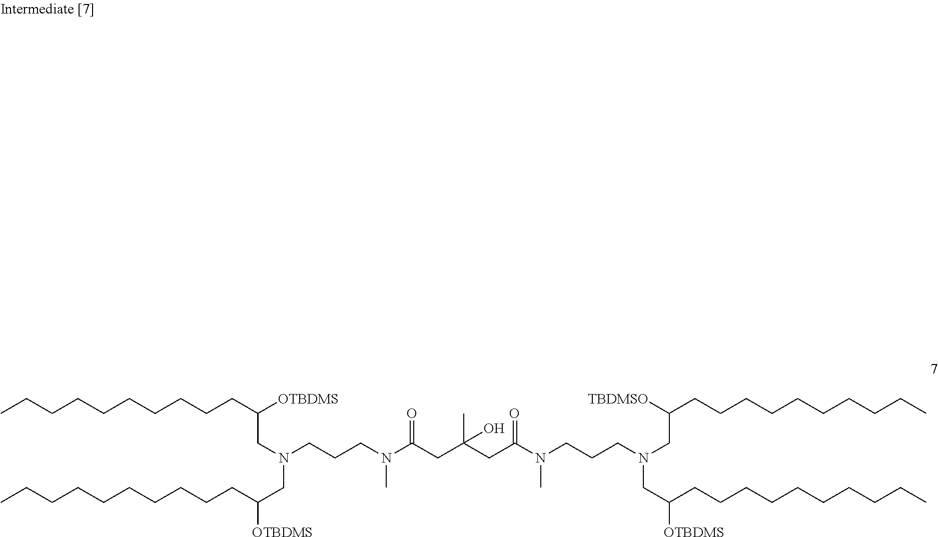 [SMILES: CCCCCCCCCCC(CN(CCCN(C)C(=O)CC(C)(O)CC(=O)N(C)CCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 20: To a stirred solution of 3-hydroxy-3-methylglutaric acid [6] (390 mg, 2.41 mmol) in dimethylformamide (30 mL) were added N-ethylbis(isopropyl)amine (1.55 g, 12.0 mmol) and HATU reagent (2.29 g, 6.0 mmol) at RT for 15 min. After this N¹,N¹-bis(2-((tert-butyldimethylsilyl)oxy)dodecyl)-N³-methylpropane-1,3-diamine [5](3.63 g, 5.29 mmol) and allow to stirred reaction at RT for 16 h. The progress of reaction was monitored by TLC and ELSD data. After completion, reaction mass quenched by cold water (10 mL) and extracted with ethyl acetate (3×25 mL), organic layer was dried over sodium sulphate and evaporate under reduced pressure to get crude and crude was purified by flash column chromatography (SiO2: 0-30% ethyl acetate in hexane) to give desired product as N¹,N⁵-bis(3-(bis(2-((tert-butyldimethylsilyl)oxy)dodecyl)amino)propyl)-3-hydroxy-N¹,N⁵,3-trimethylpentanediamide [7](1.2 g, 33.33% Yield) as pale yellow liquid.

### Result:

ELSD analysis: Purity 99.58%, Calculated Cs₆H₁₃₂N₄O₇Si₄=1495.31, Observed=1496.80 (m/z, M+H⁺).

### Synthesis of Compound LXXXIX

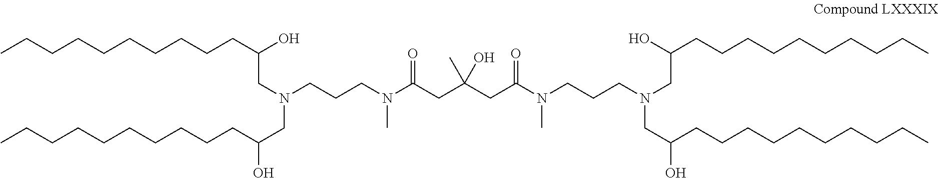 [SMILES: CCCCCCCCCCC(O)CN(CCCN(C)C(=O)CC(C)(O)CC(=O)N(C)CCCN(CC(O)CCCCCCCCCC)CC(O)CCCCCCCCCC)CC(O)CCCCCCCCCC]

As depicted in Scheme 20: To a stirred a solution of N¹,N⁵-bis(3-(bis(2-((tert-butyldimethylsilyl)oxy) dodecyl)amino) propyl)-3-hydroxy-N¹,N⁵,3-trimethylpentanediamide (0.8 g, 534 μmol) in tetrahydrofuran (8 mL) added hydrogen fluoride pyridine (1.72 mL 13.4 mmol) at cooling condition drop wise. Allow to stirred reaction mixture for 16 hr at RT, reaction progress was monitored by TLC and ELSD data. After completion, reaction mass quenched by saturated sodium bicarbonate and extracted with ethyl acetate (3×25 mL). The organic layer was combined, dried over sodium sulphate, evaporate under reduced pressure to get crude and crude was purified by flash column chromatography (SiO₂: 0-10% MeOH in DCM) to obtain N¹,N⁵-bis(3-(bis(2-hydroxydodecyl) amino)propyl)-3-hydroxy-N¹,N⁵,3-trimethylpentanediamide [Compound LXXXIX](250 mg, 45.0% Yield) as pale yellow liquid.

### Result:

¹H NMR (400 MHz, CDCl3): δ 6.20-5.70 (m, 1H), 4.35-3.79 (m, 4H), 3.59-3.52 (m, 4H), 3.51-3.37 (m, 4H), 3.02-2.98 (s, 3H), 2.92-2.91 (s, 3H), 2.86-2.20 (m, 16H), 1.80-1.67 (m, 4H), 1.50-1.38 (m, 4H), 1.40-1.20 (m, 72H), 0.89-0.86 (t, J=6.8 Hz, 12H).

ELSD analysis: Purity 99.88%, Calculated C₆₂H₁₂₆N₄O₇=1038.96, Observed=1039.65 (m/z, M+H+).

### Example 21: Synthesis of Compound XC

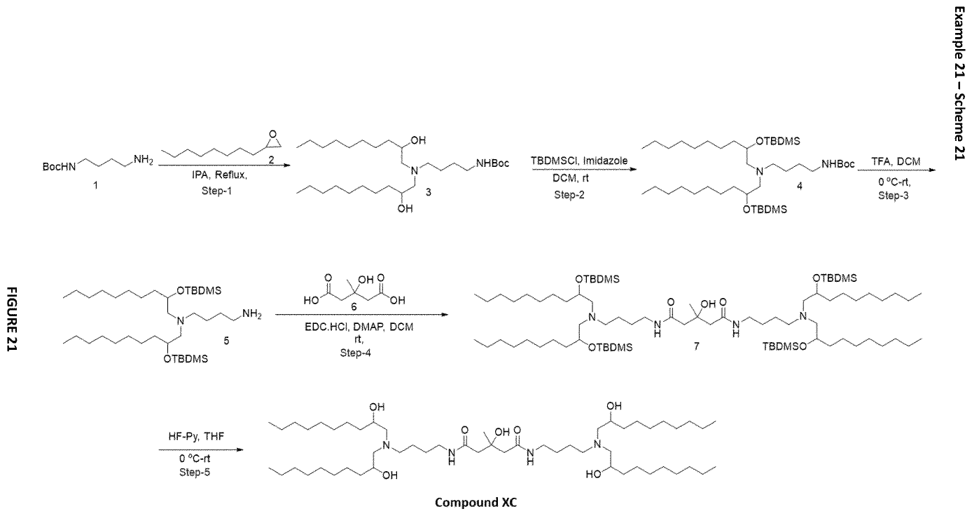

For example, the compounds of the invention may be prepared according to Scheme 21 (as depicted in FIG. 21).

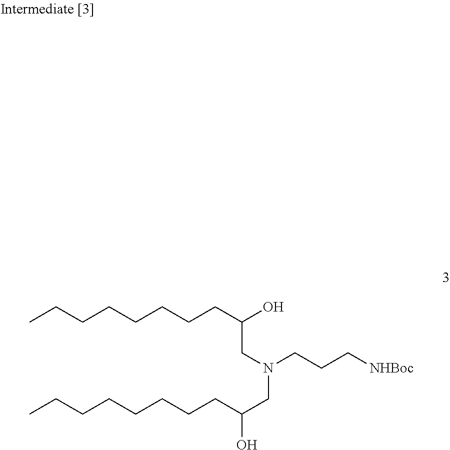 [SMILES: CCCCCCCCC(O)CN(CCCNC(=O)OC(C)(C)C)CC(O)CCCCCCCC]

As depicted in Scheme 21: To a stirred solution of tert-butyl N-(4-aminobutyl)carbamate [1](2 g, 10.6 mmol) in isopropanol (40 mL) was added ethylbis(propan-2-yl)amine (4.12 g, 31.9 mmol) and 2-octyloxirane [2](3.65 g, 23.4 mmol) at RT. The reaction mixture was heated at 90° C. for 16 hr. The progress of the reaction was checked by TLC after completion, reaction mixture concentrated under reduced pressure to obtain crude of tert-butyl (4-(bis(2-hydroxydecyl)amino)butyl)carbamate [3](5.3 g 99.62% Yield) as pale yellow liquid.

### Result:

ELSD analysis: Purity 99.11%, Calculated C₂₉H₆₀N₂O₄=500.46, Observed=501.45 (m/z, M+H+).

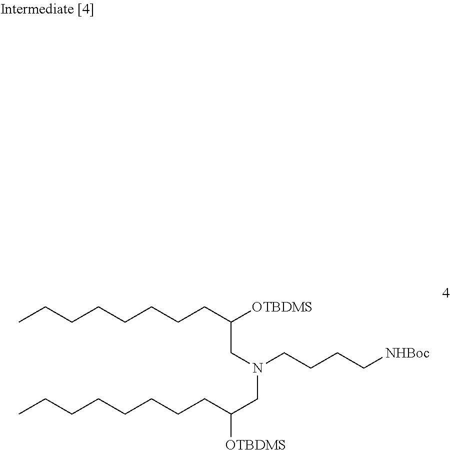 [SMILES: CCCCCCCCC(CN(CCCCNC(=O)OC(C)(C)C)CC(CCCCCCCC)O[Si](C)(C)C(C)(C)C)O[Si](C)(C)C(C)(C)C]

As depicted in Scheme 21: To the stirred solution of tert-butyl N-{4-[bis(2-hydroxydecyl)amino]butyl}carbamate (5 g, 9.98 mmol) in dichloromethane (100 mL), was added N,N-dimethylpyridin-4-amine (1.22 g, 9.98 mmol) and 1H-imidazole (10.9 g, 160 mmol). The reaction mixture was stirred for 10 minutes and added tert-butyl(chloro)dimethylsilane (15 g, 99.8 mmol). The reaction mixture was stirred at room temperature for 16 h. The progress of reaction was monitored by ELSD/TLC (Starting was consumed). Water (100 ml) was added to reaction mixture and extracted with DCM. The combined organic layer was dried by over anhydrous sodium sulphate and concentrated under reduced pressure to get crude product. The crude was purified by flash column chromatography (SiO2: 0-10% EtOAC/Hexane) to obtain tert-butyl (4-(bis(2-((tert-butyldimethylsilyl)oxy)decyl)amino)butyl)carbamate [4](7.0 g, 96.13% Yield) as a colourless liquid.

### Result:

ELSD analysis: Purity 98.45%, Calculated C₄₁H₃₃N₂O₄Si₂=728.63, Observed=729.50 (m/z, M+H+).

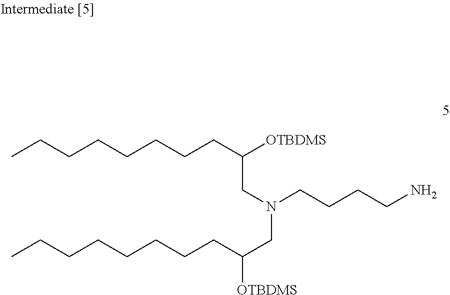 [SMILES: CCCCCCCCC(CN(CCCCN)CC(CCCCCCCC)O[Si](C)(C)C(C)(C)C)O[Si](C)(C)C(C)(C)C]

As depicted in Scheme 21: To a stirred solution of tert-butyl (4-(bis(2-((tert-butyldimethylsilyl)oxy) decyl)amino) butyl)carbamate [4](7 g, 9.6 mmol) in dichloromethane (50 mL) was added trifluoroacetic acid (7.34 mL, 96 mmol) at 0° C. The reaction was stirred at rt for 3 h. After completion, reaction mixture was concentrated under reduced pressure. The excess of trifluoroacetic was quenched by trimethylamine and concentrated to get N¹,N¹-bis(2-((tert-butyldimethylsilyl)oxy)decyl)butane-1,4-diamine [5](6.0 g crude). The crude used for next step without further purification.

### Result:

ELSD analysis: Purity 99.04%, Calculated C₃₆H₃₀N₂O₂Si₂=628.58, Observed=629.40 (m/z, M+H+).

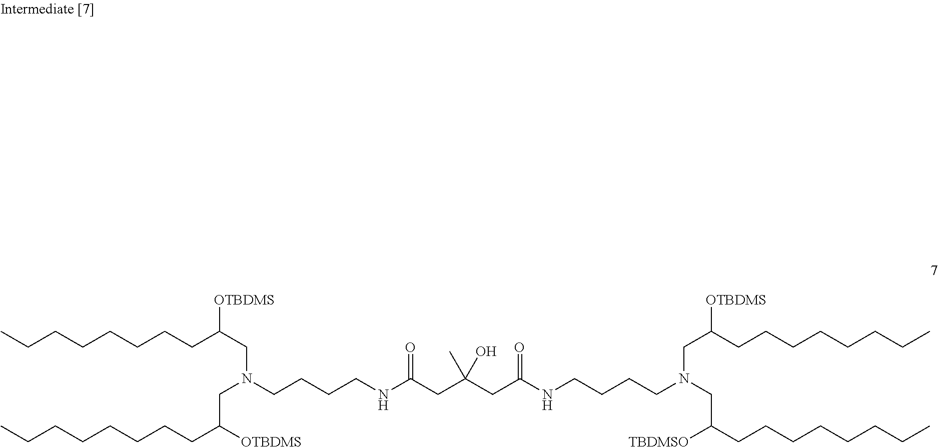 [SMILES: CCCCCCCCC(CN(CCCCNC(=O)CC(C)(O)CC(=O)NCCCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 21: To a stirred solution of 3-hydroxy-3-methylpentanedioic acid (0.3 g, 1.85 mmol) in dichloromethane (100 mL) was added EDC·HCl (887 mg, 4.63 mmol) and 4-(dimethylamino)pyridin-1-ium (456 mg, 3.7 mmol) at RT, after 15 mins N¹,N¹-bis(2-((tert-butyldimethylsilyl)oxy)decyl)butane-1,4-diamine [5](2.91 g, 4.16 mmol) was added at rt. The reaction was stirred at RT for 16 h. The progress of reaction was monitored by TLC and ELSD. The reaction mass was diluted with 30 ml water and extracted with DCM. The organic layer was washed with brine, dried over sodium sulphate and concentrated under reduced pressure to get crude compound which was purified by flash column chromatography (SiO₂: 20-35% ethyl acetate-hexane) to get N¹,N⁵-bis(4-(bis(2-((tert-butyldimethylsilyl)oxy)decyl)amino) butyl)-3-hydroxy-3-methylpentanediamide [7](455 mg, 18% Yield) as a pale yellow liquid.

### Result:

ELSD analysis: Purity 97.60%, Calculated C₇₃H₁₆₆N₄O₇Si₄=1383.18, Observed=1384.70 (m/z, M+H+).

### Synthesis of Compound XC

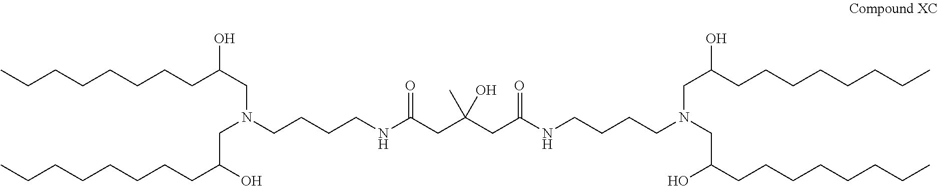 [SMILES: CCCCCCCCC(O)CN(CCCCNC(=O)CC(C)(O)CC(=O)NCCCCN(CC(O)CCCCCCCC)CC(O)CCCCCCCC)CC(O)CCCCCCCC]

As depicted in Scheme 21: To a solution of N¹,N⁵-bis(4-(bis(2-((tert-butyldimethylsilyl)oxy)decyl)amino)butyl)-3-hydroxy-3-methylpentanediamide [7](448 mg, 323 μmol) in tetrahydrofuran (5 mL), was added hydrogen fluoride pyridine (787 μL, 8.73 mmol) at 0° C. Reaction mixture was allowed to stir for overnight at RT. The progress of reaction was monitored by TLC. After SM consumed, reaction mixture was quenched with cold aq. sodium bicarbonate solution upto pH 8, and extracted with Ethyl acetate (2×50 mL). The Organic layers were dried over anhy. sodium sulphate, filtered and concentrated under reduced pressure to get crude, which was dissolved in heptane (20 mL) wash with ACN (2×5 mL). The heptane layers was collect dried under reduced pressure upto 5 mL remain, this remaining solution was filtered by 0.22 μm filter, the filtrate was concentrated under reduced pressure to obtain N¹,N⁵-bis(4-(bis(2-hydroxydecyl)amino)butyl)-3-hydroxy-3-methylpentanediamide [Compound XC](180 mg, 60% Yield) as a pale yellow liquid.

### Result:

¹H NMR (400 MHz, CDCl3): δ 7.19-6.77 (m, 2H), 5.70-5.40 (brs, 1H), 3.65-3.63 (m, 4H), 3.42-3.16 (m, 4H), 2.62-2.24 (m, 16H), 2.10-1.80 (m, 4H), 1.52-1.27 (m, 67H), 0.88-0.86 (t, J=6.8 Hz, 12H).

ELSD analysis: Purity 99.37%, Calculated C₅₄H₁₁₀N₄O₇=926.84.96, Observed=927.60 (m/z, M+H⁺).

### Example 22: Synthesis of Compound XCI

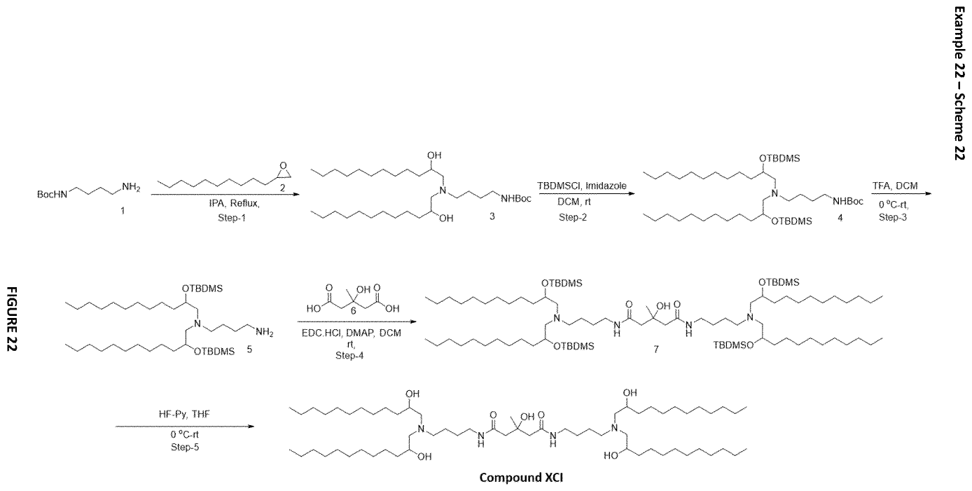

For example, the compounds of the invention may be prepared according to Scheme 22 (as depicted in FIG. 22).

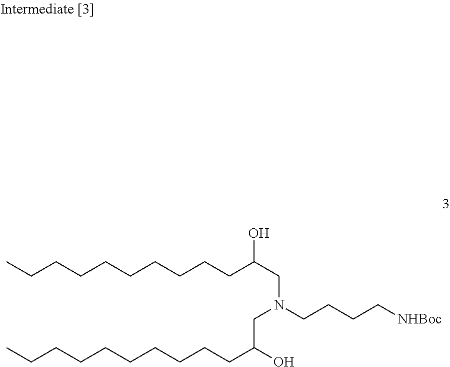 [SMILES: CCCCCCCCCCC(O)CN(CCCCNC(=O)OC(C)(C)C)CC(O)CCCCCCCCCC]

As depicted in Scheme 22: To a stirred solution of tert-butyl N-(4-aminobutyl)carbamate [1](2 g, 10.6 mmol) in isopropanol (40 mL) was added ethylbis(propan-2-yl)amine (9.28 mL, 53.1 mmol) and 2-decyloxirane [2](4.31 g, 23.4 mmol) at RT. The reaction mixture was stirred at 90° C. for 16 h. The progress of reaction was checked by TLC/ELSD. After completion, reaction mixture was concentrated under reduced pressure to give crude of tert-butyl (4-(bis(2-((tert-butyldimethylsilyl)oxy)dodecyl)amino)butyl)carbamate [3](5.9 g, 97% Yield) which was forwarded to the next step without further purification.

### Result:

ELSD analysis: Purity 97.20%, Calculated C₃₃H₆₈N₂O₄=556.52, Observed=557.50 (m/z, M+H⁺).

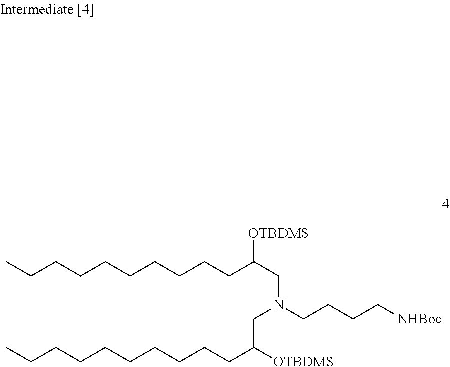 [SMILES: CCCCCCCCCCC(CN(CCCCNC(=O)OC(C)(C)C)CC(CCCCCCCCCC)O[Si](C)(C)C(C)(C)C)O[Si](C)(C)C(C)(C)C]

As depicted in Scheme 22: To a stirred solution of tert-butyl (4-(bis(2-((tert-butyldimethylsilyl)oxy)dodecyl) amino)butyl)carbamate [3](6.0 g, 10.8 mmol) in dichloromethane (100 mL) was added N,N-dimethylpyridin-4-amine (1.32 g, 10.8 mmol) and 1H-imidazole (11.7 g, 172 mmol) followed by tert-butyl(chloro)dimethylsilane (16.2 g, 108 mmol). The reaction mixture was stirred at room temperature for 16 h. The progress of reaction was monitored by TLC/ELSD (starting material was consumed). The reaction mass was dilute with water (50 mL) and extracted with DCM (3×50 mL). The combined organic layer was dried over sodium sulphate, filtered and evaporated under reduce pressure to get crude, which was purified by flash column chromatography (SiO₂: 0-10% EtOAC/Hexane) to get the tert-butyl (4-(bis(2-((tert-butyldimethylsilyl)oxy)dodecyl)amino)butyl)carbamate [4](8.0 g, 94.54% Yield) as a colourless liquid.

### Result:

ELSD analysis: Purity 99.67%, Calculated C₄₅H₉₆N₂O₄Si₂=784.69, Observed=785.55 (m/z, M+H⁺).

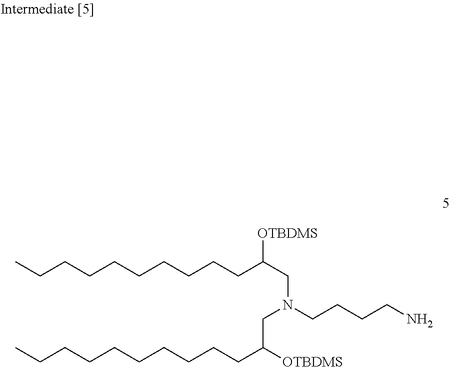 [SMILES: CCCCCCCCCCC(CN(CCCCN)CC(CCCCCCCCCC)O[Si](C)(C)C(C)(C)C)O[Si](C)(C)C(C)(C)C]

As depicted in Scheme 22: To a stirred solution of tert-butyl (4-(bis(2-((tert-butyldimethylsilyl)oxy)dodecyl) amino)butyl)carbamate [4](3.3 g, 4.2 mmol) in DCM (45 mL) was added trifluoroacetic acid (4.82 mL, 63 mmol) dropwise at 0° C. The reaction mixture was stirred at room temperature for 16 h. After completion (starting material was consumed), the reaction mixture was concentrated under reduced pressure. The crude was diluted with diethyl ether and concentrated under vacuum. After concentration the excess of trifluoracetic was quenched by trimethylamine and again concentrated under vacuum to give N¹,N¹-bis(2-((tert-butyldimethylsilyl)oxy)dodecyl)butane-1,4-diamine [5](2.8 g, 97.24% Yield). The product was used further for next step without further purification.

### Result:

ELSD analysis: Purity 99.67%, Calculated C₄₅H₉₆N₂O₄Si₂=684.64, Observed=685.55 (m/z, M+H⁺).

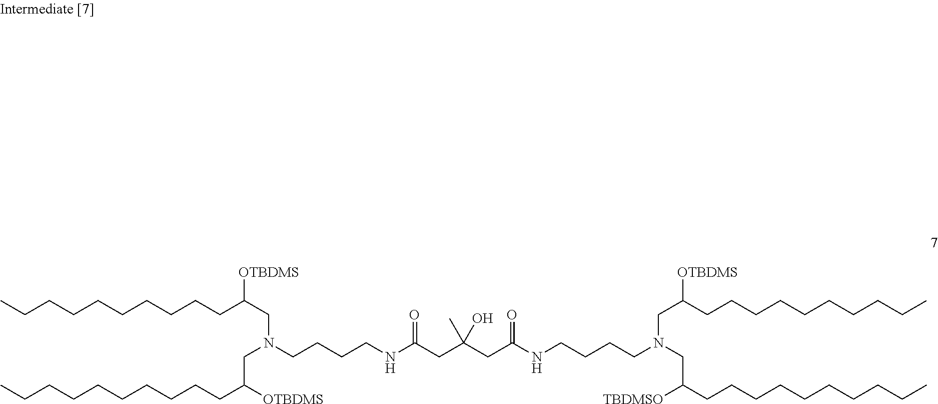 [SMILES: CCCCCCCCCCC(CN(CCCCNC(=O)CC(C)(O)CC(=O)NCCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 22: To a stirred solution of 3-hydroxy-3-methylpentanedioic acid [6](0.3 g, 1.85 mmol) in dichloromethane (60 mL) were added EDC·HCl (887 mg, 4.63 mmol) and N,N-dimethylpyridin-4-amine (497 mg, 4.07 mmol) at RT, after 15 min N¹,N¹-bis(2-((tert-butyldimethylsilyl)oxy)dodecyl)butane-1,4-diamine [5](2.79 g, 4.07 mmol) was added to the reaction mixture. The reaction mixture was stirred at RT for 16 h. The progress of reaction was monitored by TLC and ELSD. After completion, the reaction was quenched with ice cold water and extracted with ethyl acetate. The organic layer was separated, dried over sodium sulphate and evaporated under reduced pressure to get the crude compound. The crude compound was purified by flash column chromatography (SiO₂: 20-40% ethyl acetate/heptane) to afford N¹,N⁵-bis(4-(bis(2-((tert-butyldimethylsilyl)oxy)dodecyl)amino)butyl)-3-hydroxy-3-methylpentanediamide [7](550 mg, 19.34% Yield) as a pale yellow sticky liquid.

### Result:

ELSD analysis: Purity 97.48%, Calculated C₈₆H₁₃₂N₄O₇Si₄=1495.31, Observed=1495.90 (m/z, M+H⁺).

### Synthesis of Compound XCI

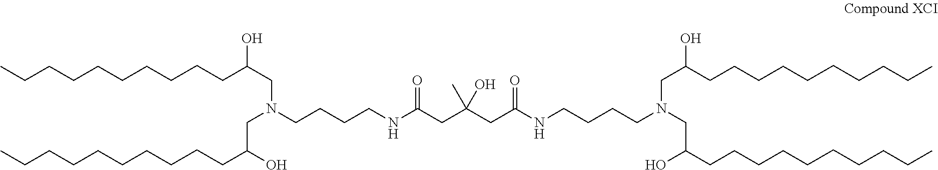 [SMILES: CCCCCCCCCCC(O)CN(CCCCNC(=O)CC(C)(O)CC(=O)NCCCCN(CC(O)CCCCCCCCCC)CC(O)CCCCCCCCCC)CC(O)CCCCCCCCCC]

As depicted in Scheme 22: To a stirred solution of N¹,N⁵-bis(4-(bis(2-((tert-butyldimethylsilyl)oxy)dodecyl) amino)butyl)-3-hydroxy-3-methylpentanediamide [7](0.5 g, 334 μmol) in tetrahydrofuran (5 mL) was added hydrogen fluoride pyridine (301 μL, 2.34 mmol) 0° C. The reaction mixture was stirred over night at RT. The progress of reaction was monitored by TLC. After completion, reaction mixture was quenched by saturated sodium bicarbonate solution up to pH 8 and extracted with ethyl acetate (3×15.0 mL). The organic layers were combine, dried over sodium sulphate anhydride, concentrated under reduced pressure. The crude was dissolved in heptane (20 mL) and washed with acetonitrile (2×5 mL). The n-heptane layer was concentrated under reduced pressure to obtain N¹,N⁵-bis(4-(bis(2-hydroxydodecyl)amino)butyl)-3-hydroxy-3-methylpentanediamide [Compound XCI](250 mg, 71.98% Yield) as a pale yellow liquid.

### Result:

¹H NMR (400 MHz, CDCl3): δ 7.15-6.72 (m, 2H), 5.70-5.30 (brs, 1H), 3.67-3.61 (m, 4H), 3.40-3.25 (m, 4H), 2.60-2.26 (m, 16H), 1.56-1.42 (m, 12H), 1.39-1.20 (m, 72H), 0.89-0.86 (t, J=6.8 Hz, 12H).

ELSD analysis: Purity 99.48%, Calculated C₆₂H₁₂₆N₄O₇=1038.96, Observed=1039.60 (m/z, M+H+).

### Example 23: Synthesis of Compound CV

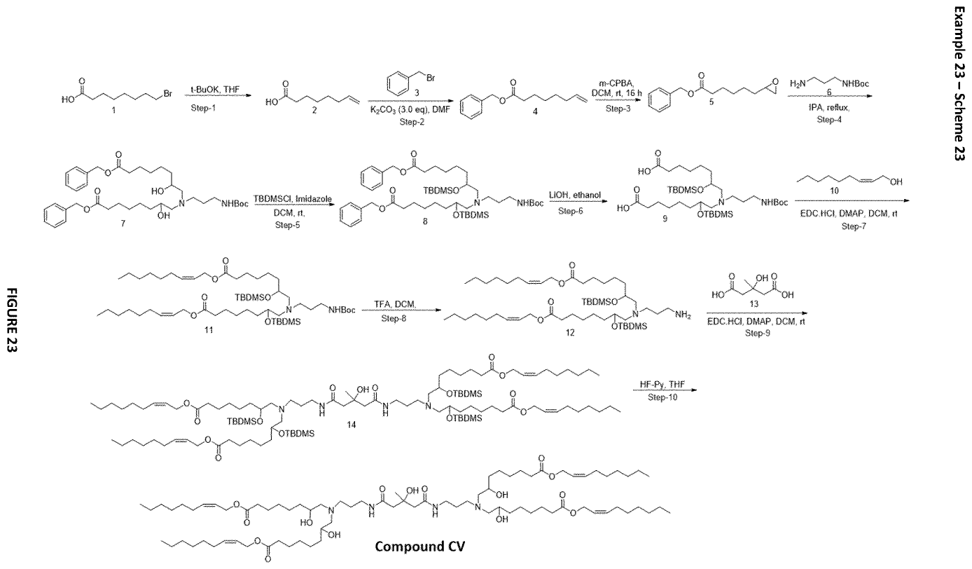

For example, the compounds of the invention may be prepared according to Scheme 23 (as depicted in FIG. 23).

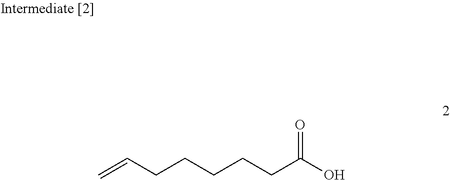

As depicted in Scheme 23: To a stirred solution of 8-bromooctanoic acid [1](100 g, 0.448 mol) in tetrahydrofuran (3.25 L) was added potassium 2-methylpropan-2-olate (226 g, 2.02 mol) at RT under nitrogen atmosphere. The reaction mixture was stirred at 90° C. for 16 h. The progress of reaction was monitored by TLC. After completion, reaction mixture quenched by 2M HCl up to pH 3 and extracted by ethyl acetate (3×250 mL). The combined organic layer was dried over anhydrous sodium sulphate, filtered and evaporated under reduced pressure to get oct-7-enoic acid [2](61.0 g, 95.76% Yield) as a light yellow liquid.

### Result:

¹H-NMR (400 MHz, CDCl₃)-δ 11.96 (s, 1H), 5.83-5.73 (m, 1H), 5.01-4.92 (m, 2H), 2.20-2.16 (t, J=7.2 Hz, 2H), 2.03-1.98 (m, 2H), 1.54-1.45 (m, 2H), 1.38-1.30 (m, 2H), 1.30-1.23 (m, 2H) ppm.

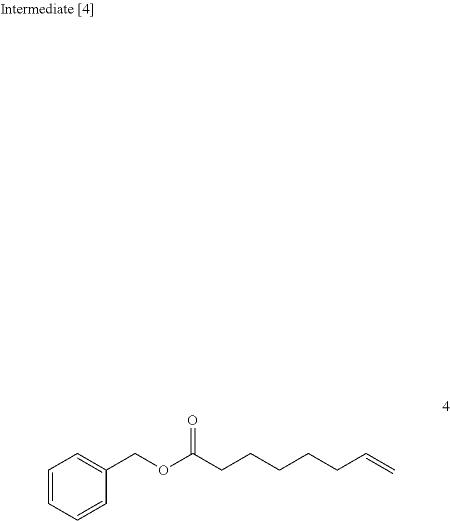 [SMILES: C=CCCCCCC(=O)OCc1ccccc1]

As depicted in Scheme 23: To a stirred solution of oct-7-enoic acid [2](9 g, 63.3 mmol) in dimethylformamide (90 mL), was added dipotassium carbonate (26.2 g, 190 mmol) and (bromomethyl)benzene [3](11.3 mL, 94.9 mmol) at rt. The reaction mixture was stirred for 16 h. The progress of reaction was monitored by TLC. After completion of the reaction, reaction mass quenched by NH₄Cl solution and extracted with ethyl acetate (3×300 mL). The organic layer was dried over anhydrous sodium sulphate and concentrated under reduced pressure to get crude. The crude was purified by flash column chromatography (SiO₂: 0-1% ethyl acetate in hexane) to give desired product as benzyl oct-7-enoate [4](10.8 g, 73.45% Yield) as a yellow liquid.

### Results:

¹H-NMR (400 MHz, CDCl₃)-δ 7.40-7.32 (m, 5H), 5.84-5.74 (m, 1H), 5.11 (s, 2H), 5.01-4.92 (m, 2H), 2.38-2.34 (t, J=7.6 Hz, 2H), 2.06-2.01 (q, J=6.8 Hz, 2H), 1.69-1.62 (m, 2H), 1.44-1.26 (m, 4H).

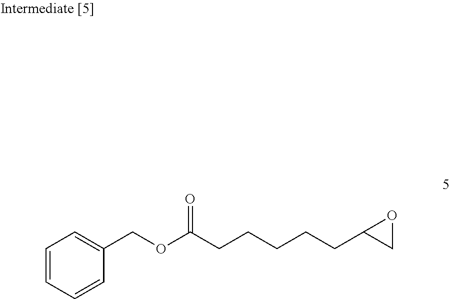 [SMILES: O=C(CCCCCC1CO1)OCc1ccccc1]

As depicted in Scheme 23: To a stirred solution of benzyl oct-7-enoate [4](10.8 g, 46.5 mmol) in dichloromethane (200 mL), was added m-chlorobenzeneperoxycarboxylic acid (16.0 g, 93 mmol). The reaction was stirred at room temperature for 16 h. The progress of reaction was monitored by TLC. After completion, reaction mixture was diluted with DCM (300 mL) and washed with cold aqueous sodium bicarbonate and organic layer was separated. The organic layer was dried over sodium sulphate and concentrated under reduced pressure to get crude. The crude product was again diluted with pentane and allow to stand for some time at low temperature. The solution was filtered and filtrate was concentrated under reduced pressure to get benzyl 6-(oxiran-2-yl)hexanoate [5](10.0 g, 86.63% Yield), which was used in next step without further purification.

(Note: The solid on sintered was quenched with aqueous sodium bicarbonate and then discarded)

### Results:

¹H-NMR (400 MHz, CDCl₃)-δ 7.43-7.30 (m, 5H), 5.11 (s, 2H), 2.91-2.87 (m, 1H), 2.75-2.73 (m, 1H), 2.46-2.44 (m, 1H), 2.39-2.35 (t, J=7.6 Hz, 2H), 1.70-1.63 (m, 2H), 1.57-1.34 (m, 6H)

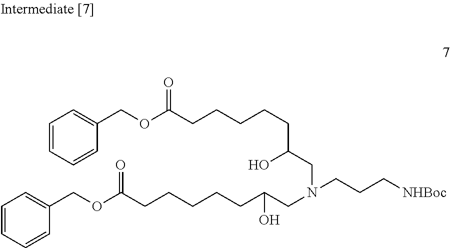 [SMILES: CC(C)(C)OC(=O)NCCCN(CC(O)CCCCCC(=O)OCc1ccccc1)CC(O)CCCCCC(=O)OCc1ccccc1]

As depicted in Scheme 23: To a stirred solution of tert-butyl N-(3-aminopropyl)carbamate [6](4.0 g, 23 mmol) in isopropanol (80 mL) add ethylbis(propan-2-yl)amine (12.2 mL, 68.9 mmol) at room temperature was added benzyl 6-(oxiran-2-yl)hexanoate [5](12.5 g, 50.5 mmol). The reaction mixture was stirred 100° C. for 16 hr. The progress of reaction was monitored by ELSD/TLC (starting material was consumed). After completion, reaction mass was concentrated under reduced pressure to get the crude product. The crude was purified by flash column chromatography (SiO₂: 0-5% MeOH-DCM) to get benzyl 8-{[8-(benzyloxy)-2-hydroxy-8-oxooctyl](3-{[(tert-butoxy)carbonyl]amino}propyl)amino}-7-hydroxyoctanoate [7](12.0 g, 77.92% Yield) as a colourless liquid.

### Result:

ELSD analysis: Purity 91.13%, Calculated C₃₈H₅₈N₂O₈=670.42, Observed=671.30 (m/z, M+H⁺).

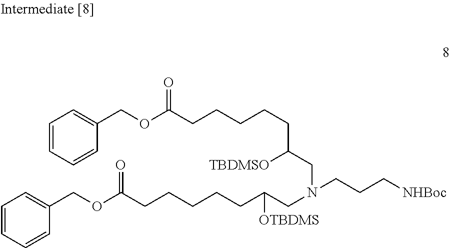 [SMILES: CC(C)(C)OC(=O)NCCCN(CC(CCCCCC(=O)OCc1ccccc1)O[Si](C)(C)C(C)(C)C)CC(CCCCCC(=O)OCc1ccccc1)O[Si](C)(C)C(C)(C)C]

As depicted in Scheme 23: To a stirred solution of benzyl 8-{[8-(benzyloxy)-2-hydroxy-8-oxooctyl](3-{[(tert-butoxy)carbonyl]amino}propyl)amino}-7-hydroxyoctanoate [7](12.0 g, 17.9 mmol) in dichloromethane (150 mL) was added 1H-imidazole (19.5 g, 286 mmol) and N,N-dimethylpyridin-4-amine (2.19 g, 17.9 mmol) at RT. The reaction mixture was stirred for 15 min and added tert-butyl(chloro)dimethylsilane (15.7 g, 104 mmol) at 0° C. The reaction mixture was stirred room temperature for 16 hr. The progress of reaction was monitored by TLC. After completion the reaction, reaction mass was diluted with water and extracted with DCM. The organic layer was collect, dried over anhydrous sodium sulphate, concentrated under reduced pressure to get crude. The crude was purified by flash column chromatography (SiO₂: 5-10% ethyl acetate in hexane) to give desired product benzyl 8-{[8-(benzyloxy)-2-[(tert-butyldimethylsilyl)oxy]-8-oxooctyl](3-{[(tert-butoxy)carbonyl]amino}propyl)amino}-7-[(tert-butyldimethylsilyl)oxy]octanoate [8](12.0 g, 74.59% Yield) as a colourless liquid.

### Result:

ELSD analysis: Purity 99.88%, Calculated C₅₀H₅₆N₂OSi₂=898.59, Observed=899.35 (m/z, M+H⁺).

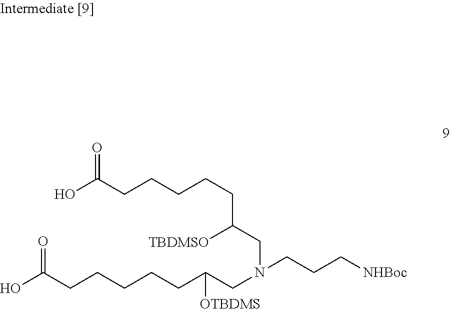 [SMILES: CC(C)(C)OC(=O)NCCCN(CC(CCCCCC(=O)O)O[Si](C)(C)C(C)(C)C)CC(CCCCCC(=O)O)O[Si](C)(C)C(C)(C)C]

As depicted in Scheme 23: To a stirred solution of benzyl 8-({7-(benzyloxycarbonyl)-2-[(tert-butyl)bis(methyl)siloxy]heptyl}[3-(tert-butoxycarbonylamino)propyl]amino)-7-[(tert-butyl)bis(methyl)siloxy]octanoate [8](6.0 g, 6.67 mmol) in tetrahydrofuran (30 mL) added 10% palladium on carbon (50% by wet moisture) (3.0 g) portion wise and stirred at room temperature. The reaction mixture was degassed and allowed to stir at room temperature for overnight under hydrogen atmosphere (balloon pressure). The progress of reaction was monitored by TLC. The reaction mixture was filtered through celite and washed with methanol two time. The filtrate was concentrated under vacuum to get 8-{[3-(tert-butoxycarbonylamino)propyl]{2-[(tert-butyl)bis(methyl)siloxy]-7-carboxyheptyl}amino}-7-[(tert-butyl)bis(methyl)siloxy]octanoic acid [9](4.7 g, 98.0% Yield) as a colourless liquid.

### Result:

ELSD analysis: Purity 99.92%, Calculated C₃₆H₇₄N₂OSi₂=718.50, Observed=719.35 (m/z, M+H⁺).

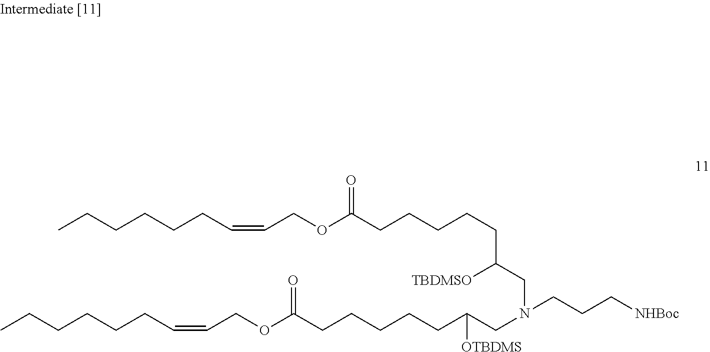 [SMILES: CCCCCC/C=C\COC(=O)CCCCCC(CN(CCCNC(=O)OC(C)(C)C)CC(CCCCCC(=O)OC/C=C\CCCCCC)O[Si](C)(C)C(C)(C)C)O[Si](C)(C)C(C)(C)C]

As depicted in Scheme 23: To stirred a solution of 8-{[3-(tert-butoxycarbonylamino)propyl]{2-[(tert-butyl)bis(methyl)siloxy]-7-carboxyheptyl}amino}-7-[(tert-butyl)bis(methyl)siloxy]octanoic acid [9](4.8 g, 6.67 mmol) in dichloromethane (50 mL) was added N,N-dimethyl-4-pyridylamine (2.45 g, 20 mmol) and EDC·HCl (3.84 g, 20 mmol). The resulting reaction mixture was stirred at RT for 15 min then added (Z)-2-nonen-1-ol [10](2.36 mL, 14 mmol). The resulting reaction mixture was stirred for 16 h at RT. The progress of reaction was monitored by TLC. After completion, the reaction mixture was diluted with dichloromethane and washed with water and brine solution. The organic layer was dried over anhydrous Na₂SO₄, filtered and concentrated. The crude was purified by flash column chromatography (SiO₂: 0-35% EtOAc in heptane) to give (Z)-2-nonenyl 8-({7-[(Z)-2-nonenyloxycarbonyl]-2-[(tert-butyl)bis(methyl)siloxy]heptyl}[3-(tert-butoxycarbonylamino)propyl]amino)-7-[(tert-butyl)bis(methyl)siloxy]octanoate [11](4.8 g, 74.17% Yield) as a colourless sticky liquid.

### Result:

ELSD analysis: Purity 99.81%, Calculated C₅₄H₁₀₆N₂O₈Si₂=966.75, Observed=967.50 (m/z, M+H⁺).

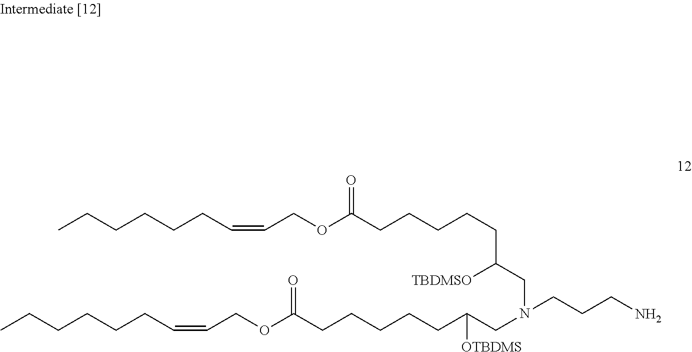 [SMILES: CCCCCC/C=C\COC(=O)CCCCCC(CN(CCCN)CC(CCCCCC(=O)OC/C=C\CCCCCC)O[Si](C)(C)C(C)(C)C)O[Si](C)(C)C(C)(C)C]

As depicted in Scheme 23: To a stirred solution of (Z)-2-nonenyl 8-({7-[(Z)-2-nonenyloxycarbonyl]-2-[(tert-butyl)bis(methyl)sioxy]heptyl}[3-(tert-butoxycarbonylamino)propyl]amino)-7-[(tert-butyl)bis(methyl)siloxy]octanoate [11](4.0 g, 4.13 mmol) in dichloromethane (40 mL) was added trifluoroacetic acid (4.75 mL, 62 mmol) at 0° C. The reaction was stirred at rt for 3 h. After completion, reaction mixture was concentrated under reduced pressure. Extra trifluoroacetic acid was quenched by DIPEA upto pH 8 and concentrated under vacuum to give di((Z)-non-2-en-1-yl) 8,8′-((3-aminopropyl)azanediyl)bis(7-((tert-butyldimethylsilyl)oxy) octanoate) [12](3.4 g, 94.81% Yield), which was used in next step without purification.

### Result:

ELSD analysis: Purity 96.40%, Calculated C₄₉H₉₈N₂O₆Si₂=866.70, Observed=867.50 (m/z, M+H⁺).

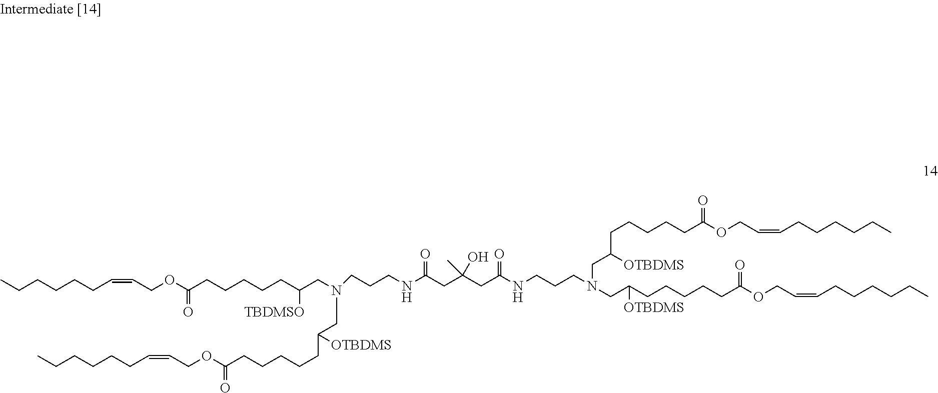 [SMILES: CCCCCC/C=C\COC(=O)CCCCCC(CN(CCCNC(=O)CC(C)(O)CC(=O)NCCCN(CC(CCCCCC(=O)OC/C=C\CCCCCC)O[Si](C)(C)C(C)(C)C)CC(CCCCCC(=O)OC/C=C\CCCCCC)O[Si](C)(C)C(C)(C)C)CC(CCCCCC(=O)OC/C=C\CCCCCC)O[Si](C)(C)C(C)(C)C)O[Si](C)(C)C(C)(C)C]

As depicted in Scheme 23: To a solution of 3-hydroxy-3-methylglutaric acid [13](0.3 g, 1.85 mmol) in dichloromethane (30 mL) were added N,N-dimethyl-4-pyridylamine (452 mg, 3.7 mmol) and 2-methyl-2,6,8-triaza-6,7-decadiene-hydrogen chloride (1/1) (709 mg, 3.7 mmol). The resulting mixture was stirred at RT for 15 min and then added (Z)-2-nonenyl 8-({7-[(Z)-2-octenyloxycarbonyl]-2-[(tert-butyl)bis(methyl)siloxy]heptyl}(3-aminopropyl)amino)-7-[(tert-butyl)bis(methyl) siloxy]octanoate [12](3.47 g, 4.07 mmol). The reaction mixture was stirred at RT for 24 h. The progress of reaction was monitor by TLC. After completion, the reaction mixture was diluted with dichloromethane and washed with water and brine solution. The organic layer was dried over anhydrous Na₂SO₄, filtered and concentrated. The crude was purified by flash column chromatography (SiO2: 0-35% EtOAc in heptane) to give (Z)-2-nonenyl 8-[(3-{4-[N-3-(bis{7-[(Z)-2-nonenyloxycarbonyl]-2-[(tert-butyl)bis(methyl)siloxy]heptyl}amino) propylcarbamoyl]-3-hydroxy-3-methylbutyrylamino}propyl){7-[(Z)-2-nonenyloxycarbonyl]-2-[(tert-butyl)bis(methyl)siloxy]heptyl}amino]-7-[(tert-butyl)bis(methyl)siloxy]octanoate [14](0.9 g, 25.87% Yield) as a colourless sticky liquid.

### Result:

ELSD analysis: Purity 99.94%, Calculated C₁₀₄H₂₀₂N₄O₁₅Si₄=1859.42, Observed=1860.10 (m/z, M+H⁺).

### Synthesis of Compound CV

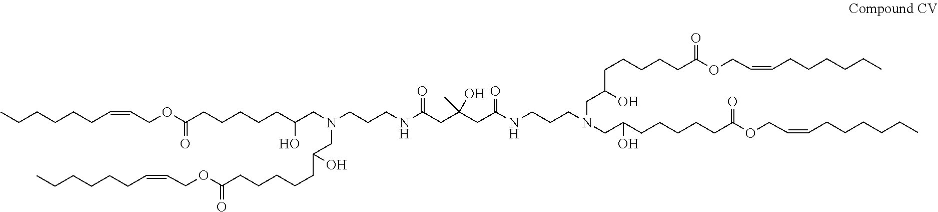 [SMILES: CCCCCC/C=C\COC(=O)CCCCCC(O)CN(CCCNC(=O)CC(C)(O)CC(=O)NCCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC]

As depicted in Scheme 23: To a stirred solution of (Z)-2-nonenyl 8-[(3-{4-[N-3-(bis{7-[(Z)-2-nonenyloxycarbonyl]-2-[(tert-butyl)bis(methyl)siloxy]heptyl}amino)propylcarbamoyl]-3-hydroxy-3-methylbutyryl amino}propyl){7-[(Z)-2-nonenyloxycarbonyl]-2-[(tert-butyl)bis(methyl)siloxy]heptyl}amino]-7-[(tert-butyl)bis(methyl)siloxy]octanoate [14](450 mg, 242 μmol) in tetrahydrofuran (5 mL) was added hydrogen fluoride pyridine (479 mg, 4.84 mmol) at 0° C. Reaction mixture was allowed to stir for overnight at RT. The progress of reaction was monitored by TLC. After completion, reaction mass was diluted with diethyl ether and water. Triethyl amine was added to maintain pH 8, separate the ether layer and washed with brine. The organic layer was dried over anhy. sodium sulphate, filtered and concentrated under reduced pressure to get crude. The crude was taken in ACN (10.0 mL) and extracted with heptane (2×20.0 mL), the heptane layer was concentrated to get (Z)-2-nonenyl 8-[(3-{4-[N-3-(bis{7-[(Z)-2-nonenyloxycarbonyl]-2-hydroxyheptyl}amino) propylcarbamoyl]-3-hydroxy-3-methylbutyrylamino}propyl){7-[(Z)-2-nonenyloxycarbonyl]-2-hydroxyheptyl}amino]-7-hydroxyoctanoate [Compound CV](0.3 g, 88.37% Yield) as a colourless sticky liquid.

### Result:

¹H NMR (400 MHz, CDCl3): δ 7.73 (brs, 2H), 5.67-5.60 (m, 4H), 5.54-5.48 (m, 4H), 4.62-4.60 (d, J=6.8 Hz, 8H), 3.77 (brs, 4H), 3.00-2.50 (m, 11H), 2.47-2.39 (m, 4H), 2.32-2.29 (t, J=7.6 Hz, 8H), 2.12-2.12 (q, J=7.2 Hz, 8H), 1.75 (brs 4H), 1.66-1.59 (m, 8H), 1.54-1.27 (m, 64H), 0.89-0.86 (t, J=6.8 Hz, 12H).

ELSD analysis: Purity 98.35%, Calculated C₈₀H₁₄₆N₄O₁₅=1403.08, Observed=1403.65 (m/z, M+H+).

### Example 24: Synthesis of Compound XCII

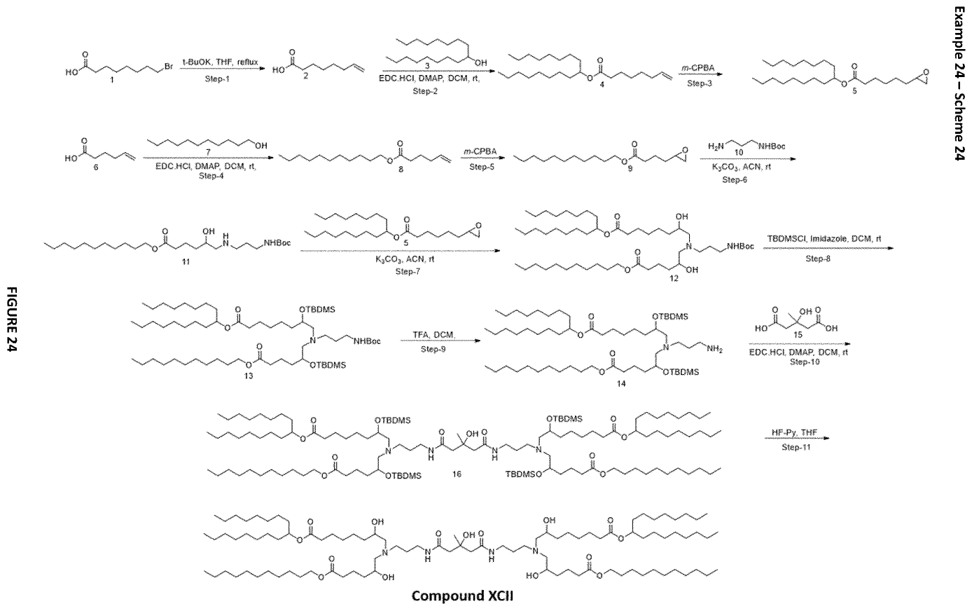

For example, the compounds of the invention may be prepared according to Scheme 24 (as depicted in FIG. 24).

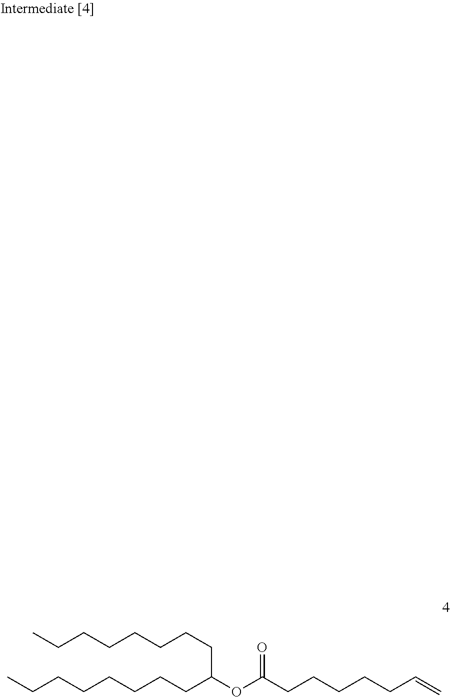 [SMILES: C=CCCCCCC(=O)OC(CCCCCCCC)CCCCCCCC]

As depicted in Scheme 24: To stirred solution of oct-7-enoic acid [2](6.5 g, 45.7 mmol) in dichloromethane (200 mL) were added 4-(dimethylamino)pyridin-1-ium (5.63 g, 45.7 mmol) and EDC·HCl (17.5 g, 91.4 mmol). After 15 min heptadecan-9-ol [3](10.6 g, 41.1 mmol) was added in reaction mixture. The reaction mixture was stirred at RT for 16 hr. The progress of reaction was monitored by TLC. After completion, reaction mass quenched by water and extracted with DCM (2×100 mL). The organic layer was dried over sodium sulphate and concentrated under reduced pressure to get crude. The crude was purified by flash column chromatography (SiO₂: 0-20% EtOAc in heptane) to give heptadecan-9-yl oct-7-enoate [4](11.0 g, 63.22% Yield) as a pale yellow liquid.

### Results:

¹H-NMR (400 MHz, CDCl₃)-δ 5.82-5.78 (m, 1H), 5.03-4.87 (m, 3H), 2.31-2.28 (t, J=7.6 Hz, 2H), 2.07-2.04 (m, 2H), 1.66-1.57 (m, 2H), 1.52-1.49 (m, 4H), 1.42-1.27 (m, 28H), 0.90-0.87 (t, J=7.2 Hz, 6H).

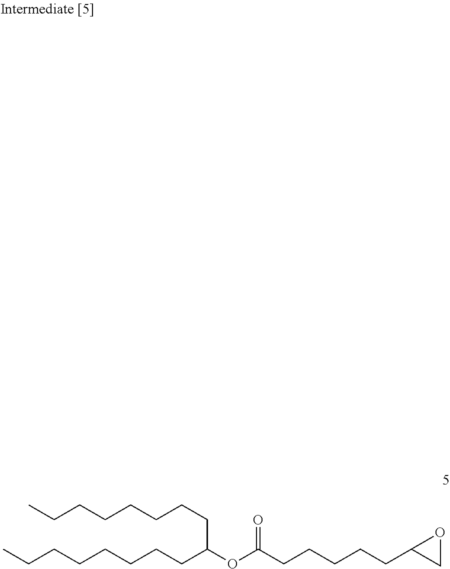 [SMILES: CCCCCCCCC(CCCCCCCC)OC(=O)CCCCCC1CO1]

As depicted in Scheme 24: To a stirred solution of heptadecan-9-yl oct-7-enoate [4](11 g, 28.9 mmol) in dichloromethane (200 L) was added 3-chlorobenzene-1-carboperoxoic acid (9.97 g, 57.8 mmol) at 0° C. under nitrogen atmosphere. The reaction mixture was stirred at RT for 16 hr. The reaction mixture was monitored by TLC. After completion, reaction mixture was quenched by saturated sodium bicarbonate and extracted with DCM (3×100 mL). The organic layer was dried over sodium sulphate and concentrated under reduced pressure to give crude. The crude product was again diluted with pentane and allow to stand for some time at low temperature. The solution was filtered and filtrate was concentrated under reduced pressure to give heptadecan-9-yl 6-(oxiran-2-yl)hexanoate [5](11.0 g, 96% Yield) as a colourless liquid.

### Results:

¹H NMR (400 MHz, CDCl₃): δ 4.90-4.83 (m, 1H), 2.92-2.88 (m, 1H), 2.75-2.73 (t, J=4.4, 1H), 2.47-2.45 (m, 1H), 2.31-2.27 (t, J=7.6 Hz, 2H), 1.68-1.60 (m, 2H), 1.58-1.34 (m, 10H), 1.29-1.26 (m, 24H), 0.89-0.86 (t, J=6.8 Hz, 6H).

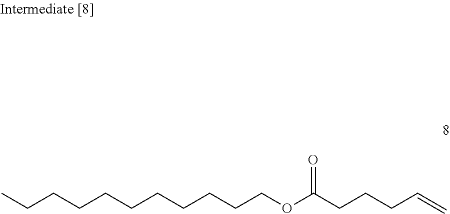 [SMILES: C=CCCCC(=O)OCCCCCCCCCCC]

As depicted in Scheme 24: To a stirred solution of hex-5-enoic acid [6](40 g, 350 mmol) in dichloromethane (800 mL) were added EDC·HCl (80.6 g, 421 mmol) and 4-(dimethylamino)pyridin-1-ium (43.2 g, 350 mmol) at room temperature. The reaction mass was stirred for 15 min and then added undecan-1-ol [7](60.4 g, 350 mmol). The reaction mixture was stirred at RT for 16 h. The progress of reaction was monitored by TLC. After completion, the reaction mass quenched with water (300 mL) and extracted with DCM (2×500 mL). The organic layer was combined, dried over sodium sulphate and evaporate under reduced pressure to get crude. The crude was dissolved in n-heptane (2×200 mL) and filtered with filter paper. The filtrate was concentrated under reduced pressure to obtain undecyl hex-5-enoate [8](92.0 g, 97.8% Yield) as a pale yellow liquid.

### Result:

¹H NMR (400 MHz, CDCl3): δ 5.83-5.76 (m, 1H), 5.06-4.99 (m, 2H), 4.09-4.05 (t, J=6.4 Hz, 2H), 2.33-2.30 (t, J=7.6 Hz, 2H), 2.13-2.08 (m, 2H), 1.78-1.70 (m, 2H), 1.66-1.59 (m, 2H), 1.31-1.23 (m, 16H), 0.90-0.88 (t, J=6.8 Hz, 3H).

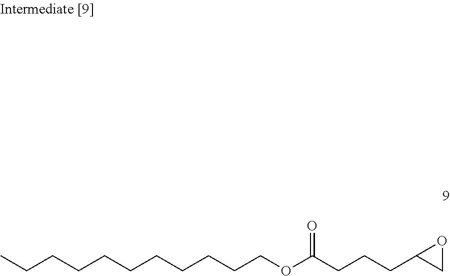 [SMILES: CCCCCCCCCCCOC(=O)CCCC1CO1]

As depicted in Scheme 24: To a stirred solution of undecyl hex-5-enoate [8](92 g, 343 mmol) in dichloromethane (1.0 L) was added 3-chlorobenzene-1-carboperoxoic acid (121 g, 0.7 mol) at 0° C. under nitrogen atmosphere. The reaction mixture was stirred at RT for 16 hr. The progress of reaction was monitored by TLC. After completion of reaction, reaction mixture was quenched by saturated sodium bicarbonate and extracted with DCM (3×200 mL). The organic layer was dried over sodium sulphate and concentrated under reduced pressure to give crude. The crude was purified by flash column chromatography (SiO₂: 0-20% ethyl acetate in n-heptane) to obtain undecyl 4-(oxiran-2-yl)butanoate [9](45.0 g, 45.18% Yield) as a colourless liquid.

¹H NMR (400 MHz, CDCl3): δ 4.08-4.05 (t, J=6.4 Hz, 2H), 2.92-2.91 (m, 1H), 2.76-2.74 (t, J=4.4, 1H), 2.48-2.46 (m, 1H), 2.39-2.35 (m, 2H), 1.82-1.77 (m, 2H), 1.65-1.56 (m, 4H), 1.30-1.26 (m, 16H), 0.89-0.86 (t, J=6.8 Hz, 3H).

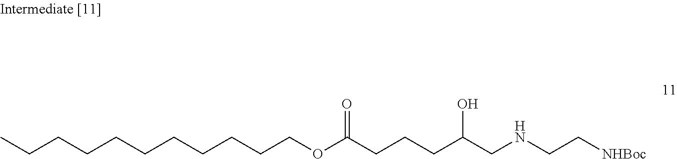 [SMILES: CCCCCCCCCCCOC(=O)CCCC(O)CNCCNC(=O)OC(C)(C)C]

As depicted in Scheme 24: To a stirred solution of tert-butyl (3-aminopropyl)carbamate [10](5 g, 28.7 mmol) in IPA (40 mL) was added N,N-diisopropylethylamine (5 mL, 28.7 mmol), undecyl 4-(2-oxiranyl)butyrate [9](8.98 g, 31.6 mmol). The reaction mixture was stirred at RT for 16 h. The progress of reaction was monitored by TLC. The reaction mixture was concentrated under reduced pressure to get crude. The crude was purified over silica gel using 0-10% MeOH in DCM to get desired product undecyl 6-[3-(tert-butoxycarbonylamino)propylamino]-5-hydroxyhexanoate [11](4.17 g, 32.0% Yield) as an off white solid.

### Result:

ELSD analysis: Purity 99.94%, Calculated C₂₅H₅₀N₂O₅=458.37, Observed=459.30 (m/z, M+H⁺).

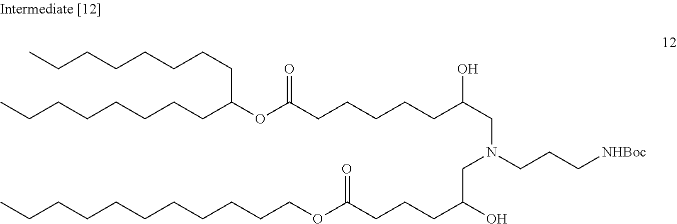 [SMILES: CCCCCCCCCCCOC(=O)CCCC(O)CN(CCCNC(=O)OC(C)(C)C)CC(O)CCCCCC(=O)OC(CCCCCCCC)CCCCCCCC]

As depicted in Scheme 24: To a stirred solution of undecyl 6-[3-(tert-butoxycarbonylamino)propylamino]-5-hydroxyhexanoate [11](4.17 g, 9.09 mmol) in isopropanol (100 mL) was added N-ethylbis(isopropyl)amine (5.88 g, 45.5 mmol) followed by the addition of 1-octylnonyl 6-(2-oxiranyl)hexanoate [5](3.61 g, 9.09 mmol) at room temperature. The s reaction mixture was stirred at 90° C. for 16 h. The progress of reaction was monitored by TLC/ELSD. After completion, isopropanol was evaporated under reduced pressure to get crude product. The crude was purified by flash column chromatography (SiO2: 0-10% MeOH/DCM) to obtain 1-octylnonyl 8-{[3-(tert-butoxycarbonylamino)propyl][2-hydroxy-5-(undecyloxycarbonyl) pentyl]amino}-7-hydroxyoctanoate [12](3.7 g, 47.58% Yield) as a colourless liquid.

### Result:

ELSD analysis: Purity 94.99%, Calculated C₅₀H₉₈N₂O₈=854.73, Observed=855.50 (m/z, M+H⁺).

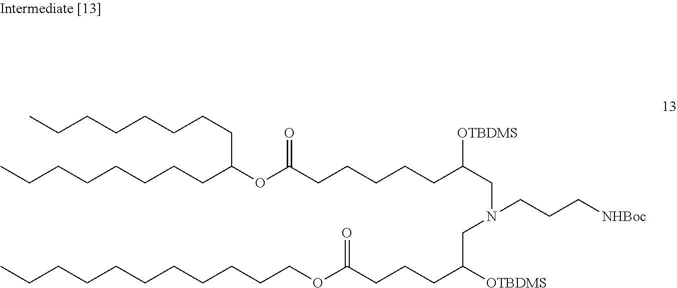 [SMILES: CCCCCCCCCCCOC(=O)CCCC(CN(CCCNC(=O)OC(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 24: To a stirred solution of 1-octylnonyl 8-{[3-(tert-butoxycarbonylamino)propyl][2-hydroxy-5-(undecyloxycarbonyl)pentyl]amino}-7-hydroxyoctanoate [12](3.6 g, 4.21 mmol) in DCM (35 mL) was added imidazole (2.29 g, 33.7 mmol) and (tert-butyl)(chloro)bis(methyl)silane (3.81 g, 25.3 mmol) at room temperature. The reaction mixture was stirred at RT for 16 h. The progress of reaction was monitored by TLC. After completion of reaction, reaction mass was diluted with water and extract by DCM (2×60 mL). The organic layer was collected, dried over anhydrous sodium sulphate, concentrated under reduced pressure to get crude. The crude was purified by flash column chromatography (SiO₂: 0-10% ethyl acetate in hexane) to give 1-octylnonyl 8-{[3-(tert-butoxycarbonylamino)propyl]{2-[(tert-butyl)bis(methyl)siloxy]-5-(undecyloxycarbonyl)pentyl}amino}-7-[(tert-butyl)bis(methyl)siloxy]octanoate [13](3.4 g, 74.53% Yield) as a colourless liquid.

### Result:

ELSD analysis: Purity 99.52%, Calculated C₆₂H₁₂₆N₂O₈Si₂=1082.91, Observed=1083.65 (m/z, M+H⁺).

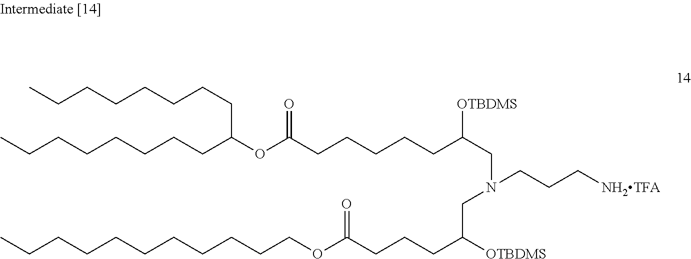

As depicted in Scheme 24: To a stirred solution of 1-octylnonyl 8-{[3-(tert-butoxycarbonylamino)propyl]{2-[(tert-butyl)bis(methyl)siloxy]-5-(undecyloxycarbonyl)pentyl}amino}-7-[(tert-butyl)bis(methyl) siloxy]octanoate [13](3.4 g, 3.14 mmol) in dichloromethane (50 mL) was added trifluoroacetic acid (2.4 mL, 31.4 mmol) at 0° C. slowly. The reaction mixture was stirred at room temperature for 16 h. The progress of reaction was monitored ELSD/TLC. After completion, reaction mass was quenched with DIPEA (maintain basic pH). Evaporate TFA by making azeotrope with dichloromethane for two to three times to get crude. This crude of 1-octylnonyl 8-[(3-aminopropyl){2-[(tert-butyl)bis(methyl)siloxy]-5-(undecyloxycarbonyl) pentyl}amino]-7-[(tert-butyl)bis(methyl)siloxy]octanoate [14](3.0 g, 97.22% Yield) as colourless liquid. The crude was used for next step without further purification.

### Result:

ELSD analysis: Purity 98.35%, Calculated C₅₇H₁₁₃N₂O₆Si₂=982.85, Observed=983.65 (m/z, M+H⁺).

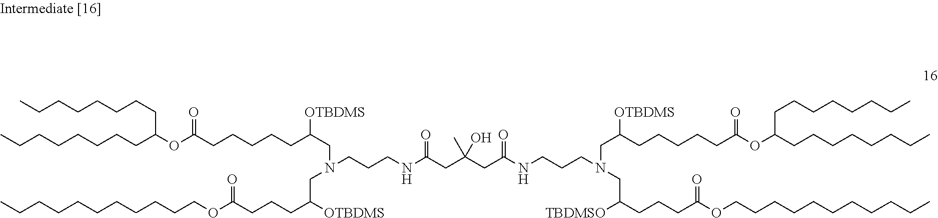 [SMILES: CCCCCCCCCCCOC(=O)CCCC(CN(CCCNC(=O)CC(C)(O)CC(=O)NCCCN(CC(CCCCCC(=O)OC(CCCCCCCC)CCCCCCCC)O[Si](C)(C)C(C)(C)C)CC(CCCC(=O)OCCCCCCCCCCC)O[Si](C)(C)C(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 24: To a stirred solution of 3-hydroxy-3-methylglutaric acid [15] (250 mg, 1.54 mmol) in dichloromethane (20 mL) was added 2-methyl-2,6,8-triaza-6,7-decadiene-hydrogen chloride (1/1) (887 mg, 4.63 mmol), N,N-dimethyl-4-pyridylamine (565 mg, 4.63 mmol). The reaction mixture was stirred for 15 min and then added 1-octylnonyl 8-[(3-aminopropyl){2-[(tert-butyl)bis(methyl)siloxy]-5-(undecyloxycarbonyl)pentyl}amino]-7-[(tert-butyl)bis(methyl)siloxy]octanoate [14](3.03 g, 3.08 mmol). The reaction mixture was stirred at room temperature for 16 h. The Progress of reaction was monitored by ELSD/TLC. After completion, reaction mass was diluted with water (50 mL) and extracted with DCM (3×80 mL). The combined organic layer was dried over anhydrous sodium sulphate and concentrated under reduced pressure to get crude product. The crude was purified by flash column chromatography (SiO₂: 0-10% ethyl acetate in heptane) to give di(heptadecan-9-yl) 7,25-bis((tert-butyldimethylsilyl)oxy)-9,23-bis(2-((tert-butyldimethylsilyl)oxy)-6-oxo-6-(undecyloxy)hexyl)-16-hydroxy-16-methyl-14,18-dioxo-9,13,19,23-tetraazahentriacontanedioate [16](1.5 g, 46.43% Yield) as a colourless liquid.

### Result:

ELSD analysis: Purity 99.43%, Calculated C₁₂₀H₂₄₂N₄O₁₅Si₄=2091.74, Observed=1047.35 (m/z, M/2+H⁺).

### Synthesis of Compound XCII

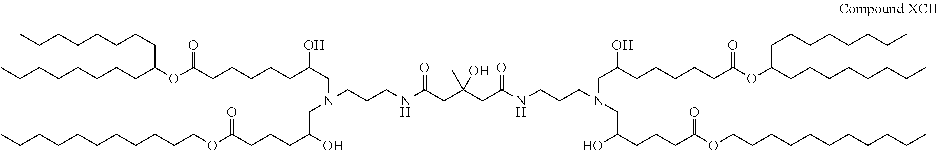 [SMILES: CCCCCCCCCCCOC(=O)CCCC(O)CN(CCCNC(=O)CC(C)(O)CC(=O)NCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC]

As depicted in Scheme 24: To stirred a solution of di(heptadecan-9-yl) 7,25-bis((tert-butyldimethylsilyl)oxy)-9,23-bis(2-((tert-butyldimethylsilyl)oxy)-6-oxo-6-(undecyloxy)hexyl)-16-hydroxy-16-methyl-14,18-dioxo-9,13,19,23-tetraazahentriacontanedioate [16](1.5 g, 716 μmol) in tetrahydrofuran (15.0 mL) was added hydrogen fluoride pyridine (2.58 mL, 28.7 mmol) drop wise at cooling condition. The reaction mixture was stirred at RT for 16 h. The progress of reaction was monitored by TLC and ELSD data. After completion, reaction mass quenched by saturated sodium bicarbonate and extracted with ethyl acetate (3×25 mL). The organic layer was combined, dried over sodium sulphate, concentrated under reduced pressure to get crude. The crude was purified by flash column chromatography (SiO₂: 0-10% MeOH in DCM) to give di(heptadecan-9-yl) 7,16,25-trihydroxy-9,23-bis(2-hydroxy-6-oxo-6-(undecyloxy)hexyl)-16-methyl-14,18-dioxo-9,13,19,23-tetraazahentriacontanedioate [Compound XCII](0.7 g, 59.7% Yield) as a colourless liquid.

### Result:

¹H NMR (400 MHz, CDCl3): δ 7.82-7.60 (m, 2H), 6.39-6.05 (m, 1H), 4.83-4.82 (m, 2H), 4.06-4.03 (t, J=6.8 Hz, 4H), 3.70-3.49 (m, 5H), 3.36 (brs, 2H), 3.14 (brs, 1H), 2.79-2.62 (m, 3H), 2.60-2.21 (m, 20H), 1.85-1.75 (m, 3H), 1.74-1.58 (m, 14H), 1.53-1.46 (m, 10H), 1.43-1.25 (m, 94H), 0.89-0.86 (t, J=6.8 Hz, 18H).

ELSD analysis: Purity 96.43%, Calculated C₉₆H₁₈₆N₄O₁₅=1635.39, Observed=1636.80 (m/z, M+H⁺).

### Example 25: Synthesis of Compound CVI

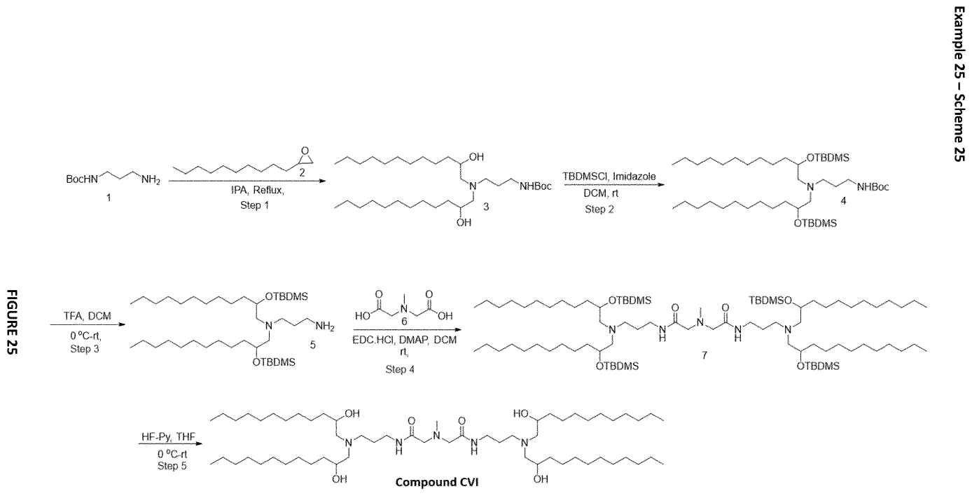

For example, the compounds of the invention may be prepared according to Scheme 25 (as depicted in FIG. 25).

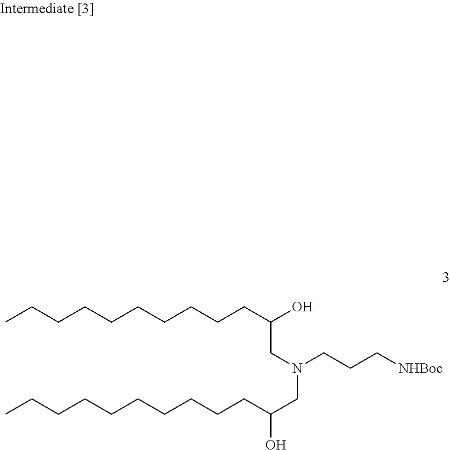 [SMILES: CCCCCCCCCCC(O)CN(CCCNC(=O)OC(C)(C)C)CC(O)CCCCCCCCCC]

As depicted in Scheme 25: To a stirred solution of tert-butyl N-(3-aminopropyl)carbamate [1](2.0 g, 11.5 mmol) and 2-decyloxirane [2](4.65 g, 25.3 mmol) in isopropanol (40 mL) was added ethylbis(propan-2-yl)amine (4 mL, 23 mmol) at room temperature. The reaction mixture was stirred at 90° C. for 16 h. The progress of reaction was monitored by ELSD and TLC. After completion, reaction mixture was cooled to room temperature and concentrated under reduced pressure to get tert-butyl N-{3-[bis(2-hydroxydodecyl)amino]propyl}carbamate [3](6.2 g, 95.98% Yield) as a colourless liquid, which was used in next step without purification.

### Result:

ELSD analysis: Purity 96.47%, Calculated C₃₂H₆₆N₂O₄=542.50, Observed=543.45 (m/z, M+H⁺).

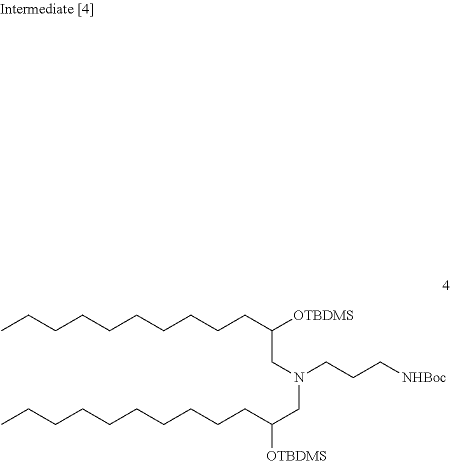 [SMILES: CCCCCCCCCCC(CN(CCCNC(=O)OC(C)(C)C)CC(CCCCCCCCCC)O[Si](C)(C)C(C)(C)C)O[Si](C)(C)C(C)(C)C]

As depicted in Scheme 25: To a stirred solution of tert-butyl N-{3-[bis(2-hydroxydodecyl)amino]propyl}carbamate [3](6 g, 11.1 mmol) in dichloromethane (60 mL) were added 1H-imidazole (12 g, 177 mmol) and N,N-dimethylpyridin-4-amine (1.35 g, 11.1 mmol) at room temperature. The reaction mixture was stirred for 10 min and then added tert-butyl(chloro)dimethylsilane (16.7 g, 111 mmol) at 0° C. The resulting mixture was stirred at room temperature for 16 h. The progress of reaction was monitored by ELSD and TLC. The reaction mixture was diluted with dichloromethane and washed with water and brine solution. The organic layer was dried over anhydrous Na₂SO₄ and concentrated. The crude residue was purified by flash column chromatography (SiO₂: 0-3% EtOAc in heptane) to obtain tert-butyl N-{3-[5,9-bis(decyl)-2,2,3,3,11,11,12,12-octamethyl-4,10-dioxa-7-aza-3,11-disilatridecan-7-yl]propyl}carbamate [4](7.0 g, 82.06% Yield) as a colourless liquid.

### Result:

ELSD analysis: Purity 99.94%, Calculated C₄₄H₉₄N₂O₄Si₂=770.68, Observed=771.50 (m/z, M+H⁺).

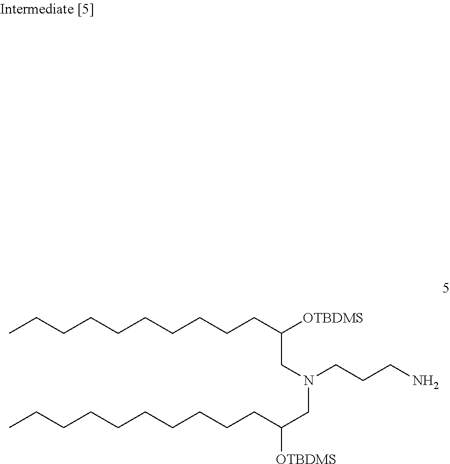 [SMILES: CCCCCCCCCCC(CN(CCCN)CC(CCCCCCCCCC)O[Si](C)(C)C(C)(C)C)O[Si](C)(C)C(C)(C)C]

As depicted in Scheme 25: To a stirred solution of tert-butyl N-{3-[5,9-bis(decyl)-2,2,3,3,11,11,12,12-octamethyl-4,10-dioxa-7-aza-3,11-disilatridecan-7-yl]propyl}carbamate [4] (6.0 g, 7.78 mmol) in dichloromethane (100 mL) was added trifluoroacetic acid (13.3 g, 117 mmol) at 0° C. Reaction mass stirred at RT for 4 h. The progress of reaction was monitored by TLC (starting material consumed). After completion, reaction mass concentrated under reduced pressure to give crude compound. The crude was dissolved in diethyl ether and concentrated under vacuum to give 7-(3-aminopropyl)-5,9-bis(decyl)-2,2,3,3,11,11,12,12-octamethyl-4,10-dioxa-7-aza-3,11-disilatridecane (5.5 gm crude) as a viscous liquid. After that the product was treated with triethylamine to quench trifluoroacetic acid to get 7-(3-aminopropyl)-5,9-bis(decyl)-2,2,3,3,11,11,12,12-octamethyl-4,10-dioxa-7-aza-3,11-disilatridecane [5](5.5 g crude). The product was used further for next step.

### Result:

ELSD analysis: Purity 99.74%, Calculated C₃₉H₈₆N₂O₂Si₂=670.62, Observed=671.50 (m/z, M+H⁺).

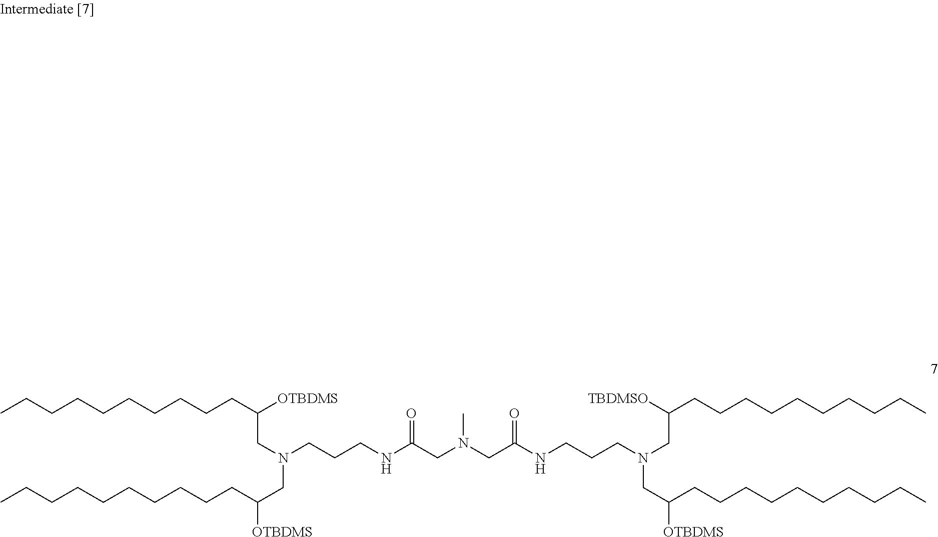 [SMILES: CCCCCCCCCCC(CN(CCCNC(=O)CN(C)CC(=O)NCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 25: To a stirred solution of 2-[(carboxymethyl)(methyl)amino]acetic acid [6](250 mg, 1.7 mmol) in dichloromethane (50 mL) was added 4-(dimethylamino)pyridin-1-ium (419 mg, 3.4 mmol), followed by EDC·HCl (814 mg, 4.25 mmol). The reaction mixture was stirred at RT for 15 min, then added 7-(3-aminopropyl)-5,9-bis(decyl)-2,2,3,3,11,11,12,12-octamethyl-4,10-dioxa-7-aza-3,11-disilatridecane [5](2.51 g, 3.74 mmol). The resulting reaction mixture was stirred for overnight at RT. The progress of reaction was monitored by TLC. The reaction mixture was diluted with dichloromethane (50.0 mL) and washed with water (50.0 mL) and brine solution (50.0 mL). The organic layer was dried over anhydrous Na₂SO₄, filtered and concentrated. The crude was purified by flash column chromatography (SiO₂: 0-35% EtOAc in heptane) to give 2,2′-(methylazanediyl)bis(N-(3-(bis(2-((tert-butyldimethylsilyl)oxy)dodecyl)amino)propyl)acetamide) [7](350 mg, 14.17% Yield) as a pale yellow liquid.

### Result:

ELSD analysis: Purity 99.16%, Calculated C₃₃H₇₇N₅O₆Si₄=1452.28, Observed=1453.90 (m/z, M+H⁺).

### Synthesis of Compound CVI

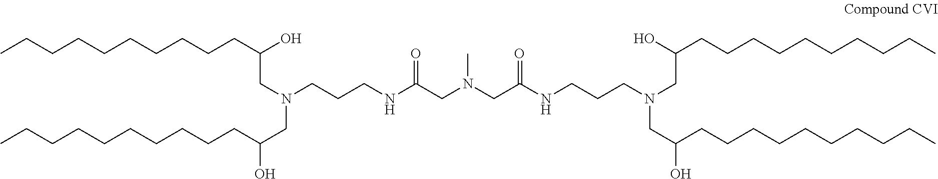 [SMILES: CCCCCCCCCCC(O)CN(CCCNC(=O)CN(C)CC(=O)NCCCN(CC(O)CCCCCCCCCC)CC(O)CCCCCCCCCC)CC(O)CCCCCCCCCC]

As depicted in Scheme 25: To a stirred solution of 2,2′-(methylazanediyl)bis(N-(3-(bis(2-((tert-butyldimethylsilyl)oxy) dodecyl)amino)propyl)acetamide) [7](0.3 g, 206 μmol) in tetrahydrofuran (3 mL) was added hydrogen fluoride pyridine (205 mg, 2.06 mmol) at 0° C. Reaction mixture was stirred for overnight at RT. The progress of reaction was monitor by TLC. After completion, reaction mixture was quenched with cold aq. sodium bicarbonate solution upto pH 8, and extracted with Ethyl acetate (2×50 mL). Organic layer was dried over anhy. sodium sulphate, filtered and concentrated under reduced pressure to get crude. The crude was dissolved in heptane (20 mL) washed with ACN (2×5 mL). The heptane layers were collect and concentrated under reduced pressure upto 5 mL. The remaining solution was filtered by 0.22 μm filter and the filtrate was concentrated under reduced pressure to obtain 2,2′-(methylazanediyl)bis(N-(3-(bis(2-hydroxydodecyl)amino)propyl)acetamide) (180 mg, 87.52% Yield) as a colourless liquid.

### Result:

¹H NMR (400 MHz, CDCl3): δ 7.76 (brs, 2H), 3.70-3.60 (brs, 4H), 3.50-3.30 (m, 4H), 3.08 (s, 4H), 2.69-2.62 (m, 3H), 2.58-2.54 (m, 2H), 2.48-2.42 (m, 4H), 2.36-2.32 (m, 4H), 2.28-2.25 (m, 2H), 1.72 (brs, 6H), 1.48-1.20 (m, 74H), 0.89-0.86 (t, J=6.8 Hz, 12H).

ELSD analysis: Purity 99.89%, Calculated C₅₉H₁₂₁N₅O₆=995.93, Observed=996.70 (m/z, M+H+).

### Example 26: Synthesis of Compound LXXXVII

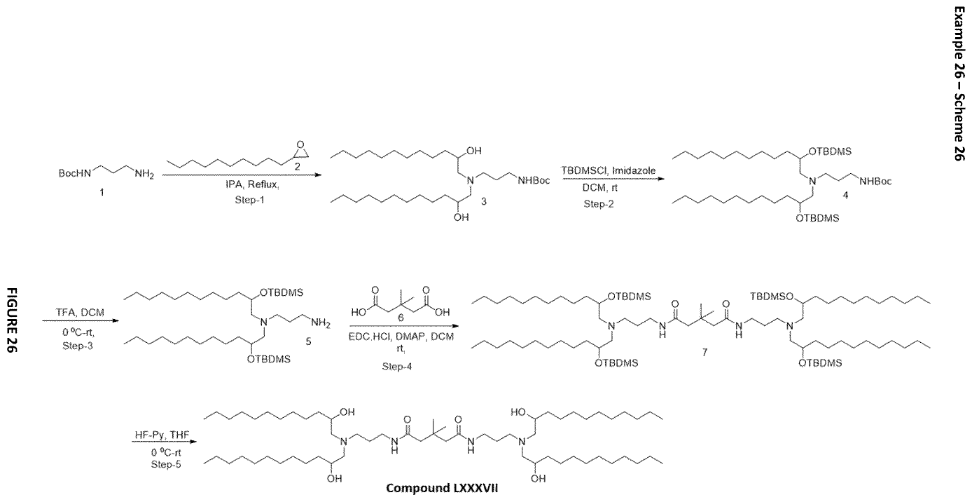

For example, the compounds of the invention may be prepared according to Scheme 26 (as depicted in FIG. 26).

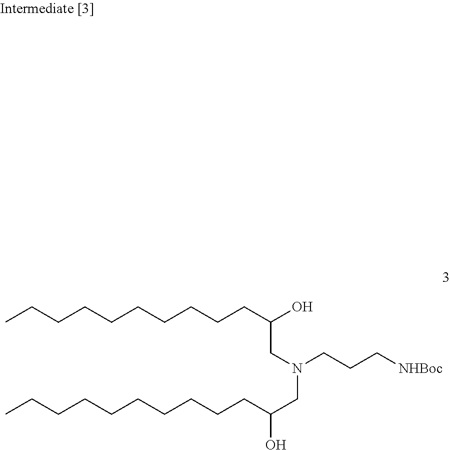 [SMILES: CCCCCCCCCCC(O)CN(CCCNC(=O)OC(C)(C)C)CC(O)CCCCCCCCCC]

As depicted in Scheme 26: To a stirred solution of tert-butyl N-(3-aminopropyl)carbamate [1](2.0 g, 11.5 mmol) and 2-decyloxirane [2](4.65 g, 25.3 mmol) in isopropanol (40 mL) was added ethylbis(propan-2-yl)amine (4 mL, 23 mmol) at room temperature. The reaction mixture was stirred at 90° C. for 16 h. The progress of reaction was monitored by ELSD and TLC. After completion, reaction mixture was cooled to room temperature and concentrated under reduced pressure to get tert-butyl N-{3-[bis(2-hydroxydodecyl)amino]propyl}carbamate [3](6.2 g, 95.98% Yield) as a colourless liquid, which was used in next step without purification.

### Result:

ELSD analysis: Purity 96.47%, Calculated C₃₂H₆₆N₂O₄=542.50, Observed=543.45 (m/z, M+H⁺).

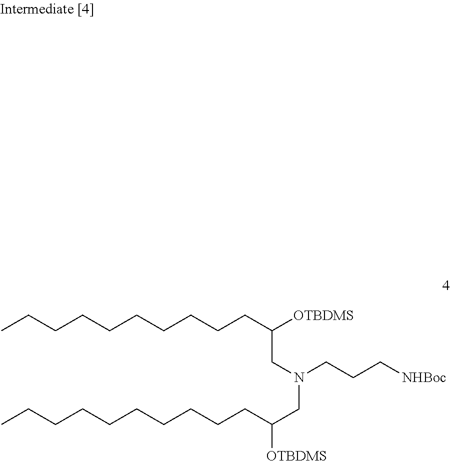 [SMILES: CCCCCCCCCCC(CN(CCCNC(=O)OC(C)(C)C)CC(CCCCCCCCCC)O[Si](C)(C)C(C)(C)C)O[Si](C)(C)C(C)(C)C]

As depicted in Scheme 26: To a stirred solution of tert-butyl N-{3-[bis(2-hydroxydodecyl)amino]propyl}carbamate [3](6 g, 11.1 mmol) in dichloromethane (60 mL) were added 1H-imidazole (12 g, 177 mmol) and N,N-dimethylpyridin-4-amine (1.35 g, 11.1 mmol) at room temperature. The reaction mixture was stirred for 10 min and then added tert-butyl(chloro)dimethylsilane (16.7 g, 111 mmol) at 0° C. The resulting mixture was stirred at room temperature for 16 h. The progress of reaction was monitored by ELSD and TLC. The reaction mixture was diluted with dichloromethane and washed with water and brine solution. The organic layer was dried over anhydrous Na₂SO₄ and concentrated. The crude residue was purified by flash column chromatography (SiO₂: 0-3% EtOAc in heptane) to obtain tert-butyl N-{3-[5,9-bis(decyl)-2,2,3,3,11,11,12,12-octamethyl-4,10-dioxa-7-aza-3,11-disilatridecan-7-yl]propyl}carbamate [4](7.0 g, 82.06% Yield) as a colourless liquid.

### Result:

ELSD analysis: Purity 99.94%, Calculated C₄₄H₉₄N₂O₄Si₂=770.68, Observed=771.50 (m/z, M+H⁺).

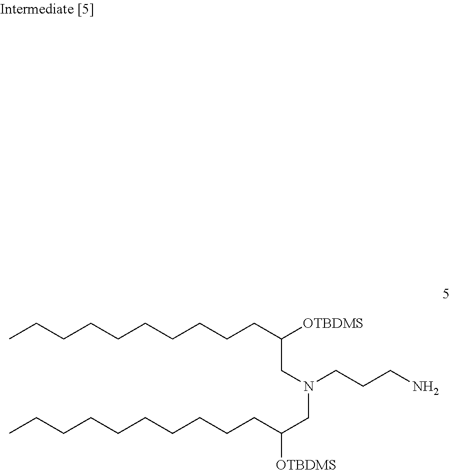 [SMILES: CCCCCCCCCCC(CN(CCCN)CC(CCCCCCCCCC)O[Si](C)(C)C(C)(C)C)O[Si](C)(C)C(C)(C)C]

As depicted in Scheme 26: To a stirred solution of tert-butyl N-{3-[5,9-bis(decyl)-2,2,3,3,11,11,12,12-octamethyl-4,10-dioxa-7-aza-3,11-disilatridecan-7-yl]propyl}carbamate (6.0 g, 7.78 mmol) in dichloromethane (100 mL) was added trifluoroacetic acid (13.3 g, 117 mmol) at 0° C. Reaction mass stirred at RT for 4 h. The progress of reaction was monitored by TLC (starting material consumed). After completion, reaction mass concentrated under reduced pressure to give crude compound. The crude was dissolved in diethyl ether and concentrated under vacuum to give 7-(3-aminopropyl)-5,9-bis(decyl)-2,2,3,3,11,11,12,12-octamethyl-4,10-dioxa-7-aza-3,11-disilatridecane (5.5 gm crude) as a viscous liquid. After that the product was treated with triethylamine to quench trifluoroacetic acid to get 7-(3-aminopropyl)-5,9-bis(decyl)-2,2,3,3,11,11,12,12-octamethyl-4,10-dioxa-7-aza-3,11-disilatridecane [5](5.5 g crude). The product was used further for next step.

### Result:

ELSD analysis: Purity 99.74%, Calculated C₃₉H₈₆N₂O₂Si₂=670.62, Observed=671.50 (m/z, M+H⁺).

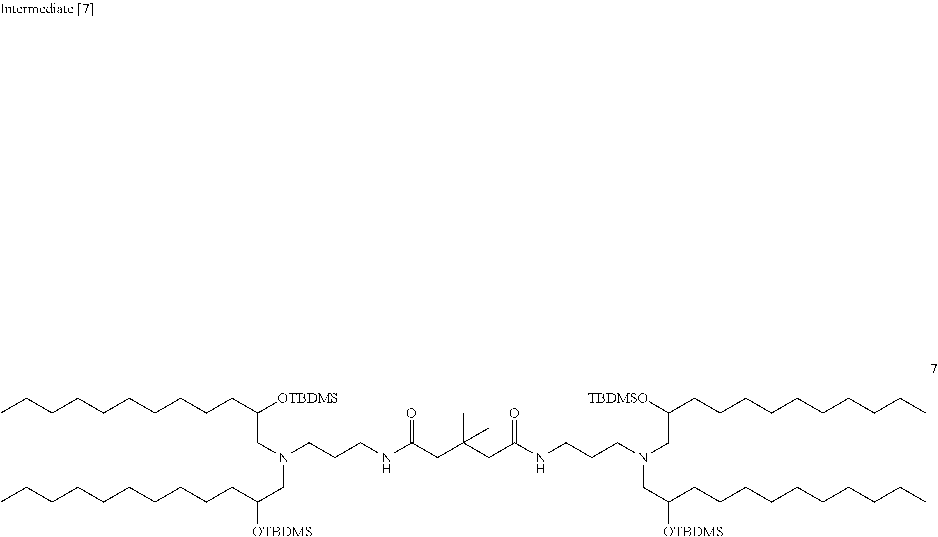 [SMILES: CCCCCCCCCCC(CN(CCCNC(=O)CC(C)(C)CC(=O)NCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 26: To a stirred solution of 3,3-dimethylpentanedioic acid [6](0.3 g, 1.87 mmol) in dichloromethane (20 mL), were added 4-(dimethylamino)pyridin-1-ium (461 mg, 3.75 mmol) and EDC·HCl (898 mg, 4.68 mmol)® The reaction mixture was stirred for 10 min at RT, then added 7-(3-aminopropyl)-5,9-bis(decyl)-2,2,3,3,11,11,12,12-octamethyl-4,10-dioxa-7-aza-3,11-disilatridecane [5](2.64 g, 3.93 mmol). The reaction mixture was stirred at room temperature for 16 h. The progress of reaction was monitored by ELSD/TLC (starting material was consumed). After completion, the reaction mixture was quenched with water/brine solution (50 mL) and extracted with dichloromethane (2×50 mL). The combined organic layer was dried anhydrous sodium sulphate and concentrated under reduced pressure to get crude product. The crude was purified by flash column chromatography (SiO₂: 0-40% EtOAC/Hexane) to obtain N,N′-bis({3-[5,9-bis(decyl)-2,2,3,3,11,11,12,12-octamethyl-4,10-dioxa-7-aza-3,11-disilatridecan-7-yl]propyl})-3,3-dimethylpentanediamide [7](1.3 g, 47.32% Yield) as a pale yellow liquid

### Result:

ELSD analysis: Purity 78.91%, Calculated Cs₅H₁₃₀N₄O₆Si₄=1465.30, Observed=1466.05 (m/z, M+H⁺).

### Synthesis of Compound LXXXVII

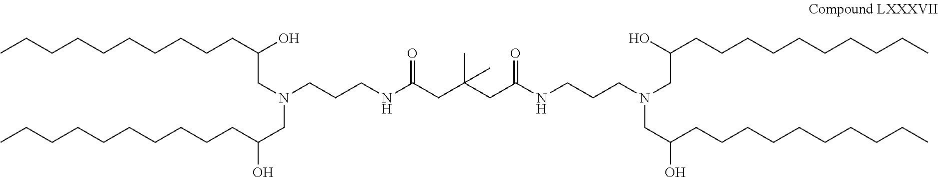 [SMILES: CCCCCCCCCCC(O)CN(CCCNC(=O)CC(C)(C)CC(=O)NCCCN(CC(O)CCCCCCCCCC)CC(O)CCCCCCCCCC)CC(O)CCCCCCCCCC]

As depicted in Scheme 26: To stirred solution of N,N′-bis({3-[5,9-bis(decyl)-2,2,3,3,11,11,12,12-octamethyl-4,10-dioxa-7-aza-3,11-disilatridecan-7-yl]propyl})-3,3-dimethylpentanediamide [7](1.3 g, 886 μmol) in tetrahydrofuran (13 mL) was added hydrogen fluoride pyridine (2.81 g, 28.4 mmol) drop wise at cooling condition. The reaction mixture was stirred for 16 hr at RT. The progress of reaction was monitored by TLC and ELSD data. After completion, reaction mass was quenched with saturated sodium bicarbonate and extracted with ethyl acetate (3×10 mL). The organic layers were combined and dried over sodium sulphate, concentrated under reduced pressure to get crude. The crude was purified by flash column chromatography (SiO₂: 0-10% methanol in dichloromethane) to obtain N,N′-bis({3-[bis(2-hydroxydodecyl)amino]propyl})-3,3-dimethylpentanediamide [Compound LXXXVII](0.5 g, 56% Yield) as a pale yellow liquid.

### Result:

¹H NMR (400 MHz, CDCl3): δ 7.52-7.43 (m, 2H), 3.70-3.60 (brs, 4H), 3.49-3.21 (m, 4H), 2.68-2.63 (m, 4H), 2.58-2.53 (m, 2H), 2.48-2.42 (m, 6H), 2.31-2.18 (m, 8H), 1.72-1.64 (m, 4H), 1.46-1.34 (m, 8H), 1.25 (m, 64H), 1.06-1.05 (m, 6H) 0.89-0.86 (t, J=6.8 Hz, 12H).

ELSD analysis: Purity 99.95%, Calculated C₆₁H₁₂₄N₄O₆=1008.95, Observed=1009.75 (m/z, M+H⁺).

### Example 27: Synthesis of Compound LXXV

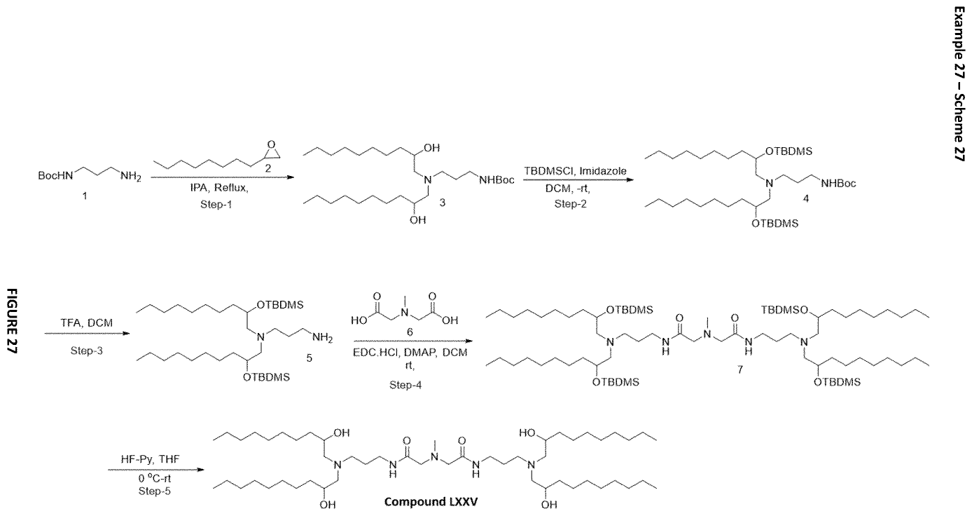

For example, the compounds of the invention may be prepared according to Scheme 27 (as depicted in FIG. 27).

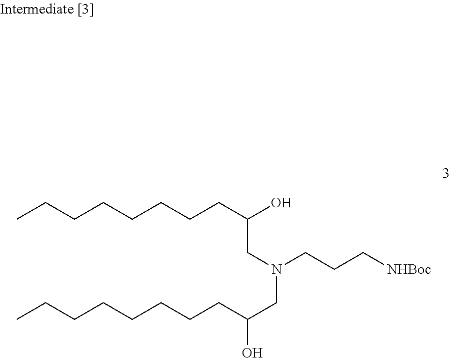 [SMILES: CCCCCCCCC(O)CN(CCCNC(=O)OC(C)(C)C)CC(O)CCCCCCCC]

As depicted in Scheme 27: To a stirred solution of tert-butyl (3-aminopropyl)carbamate [1](2.0 g, 11.5 mmol) and 2-octyloxirane [2](3.95 g, 25.3 mmol) in isopropanol (40 mL) was added ethylbis(propan-2-yl)amine (1.48 g, 11.5 mmol). The resultant reaction mixture was stirred for 16 h at 90° C. The reaction progress was monitored by TLC and ELSD. Starting material was consumed completely. Reaction mass was concentrated under reduced pressure to get crude compound, which was purified by silica gel flash column chromatography (0-5% MeOH in DCM) to give tert-butyl N-{3-[bis(2-hydroxydecyl)amino]propyl}carbamate [3](5.8 g, 93.4% yield) as a colourless liquid

### Result:

ELSD analysis: Purity 96.6%, Calculated C₂₈H₅₈N₂O₄=486.44, Observed=487.40 (m/z, M+H⁺).

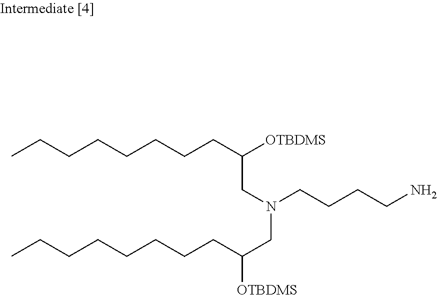 [SMILES: CCCCCCCCC(CN(CCCCN)CC(CCCCCCCC)O[Si](C)(C)C(C)(C)C)O[Si](C)(C)C(C)(C)C]

As depicted in Scheme 27: To a stirred solution of 3-[bis(2-hydroxydecyl)amino]propylamino-tert-butylformylate [3](4.7 g, 9.66 mmol) in dichloromethane (100 mL), was added imidazole (5.26 g, 77.2 mmol) followed by the addition of (tert-butyl)(chloro)bis(methyl)silane (8.73 g, 57.9 mmol) at room temperature. The reaction mixture was stirred at room temperature for 16 h. The progress of reaction was monitored by ELSD/TLC. After completion (SM was almost consumed), reaction mass was diluted with water (20 ml) and extracted with DCM (2×100 mL). The combined organic layer was dried over anhydrous sodium sulphate and concentrated under reduced pressure to get crude. The crude was then purified by flash column chromatography (SiO₂: 0-20% EtOAc/Hexane) to get 3-(bis{2-[(tert-butyl)bis(methyl)siloxy]decyl}amino)propylamino-tert-butylformylate [4](5.4 g, 78.19% Yield) as a pale yellow liquid.

### Result:

ELSD analysis: Purity 99.78%, Calculated C₄₀H₅₆N₂O₄Si₂=714.61, Observed=715.65 (m/z, M+H+).

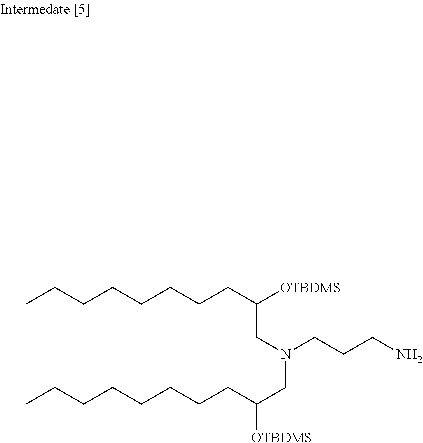 [SMILES: CCCCCCCCC(CN(CCCN)CC(CCCCCCCC)O[Si](C)(C)C(C)(C)C)O[Si](C)(C)C(C)(C)C]

As depicted in Scheme 27: To a stirred solution of 3-(bis{2-[(tert-butyl)bis(methyl)siloxy]decyl}amino)propylamino-tert-butylformylate [4](3.4 g, 4.75 mmol) in dichloromethane (30 mL) was added trifluoroacetic acid (3.64 mL, 10 eq., 47.5 mmol) at 0° C. The reaction mixture was stirred at RT for 6 h. The progress of reaction was monitored by TLC/ELSD (SM Consumed). After completion of reaction, reaction mixture was concentrated under reduced pressure. The crude was dissolved in DCM and concentrated under high vacuum to obtain 3-(bis{2-[(tert-butyl)bis(methyl) siloxy]decyl}amino)-1-propanamine-trifluoroacetic acid (1/1) [5](3.46 g, 98.0% Yield) as a pale yellow viscous liquid.

### Result:

ELSD analysis: Purity 99.51%, Calculated C₃₅H₇₃N₂O₂Si₂=614.56, Observed=615.60 (m/z, M+H+).

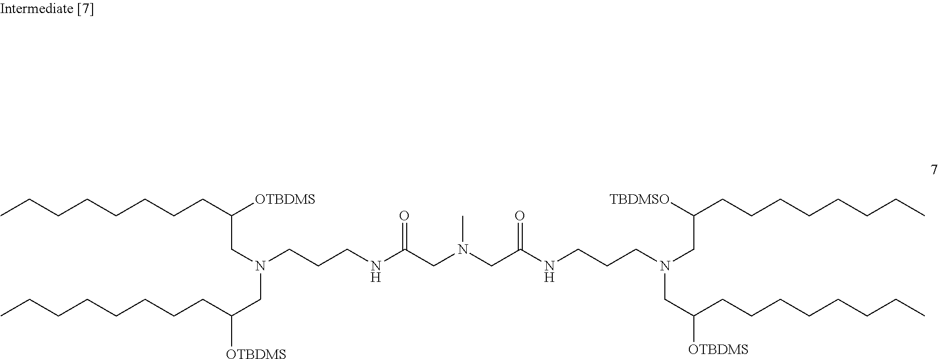 [SMILES: CCCCCCCCC(CN(CCCNC(=O)CN(C)CC(=O)NCCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 27: To a solution of [(carboxymethyl)-N-methylamino]acetic acid [6](347 mg, 2.36 mmol) in dichloromethane (50 mL) was added N,N-dimethyl-4-pyridylamine (864 mg, 7.07 mmol) and EDC·HCl (1.36 g, 7.07 mmol). The reaction mixture was stirred at RT for 15 min and then added 3-(bis{2-[(tert-butyl)bis(methyl)siloxy]decyl}amino)-1-propanamine-trifluoroacetic acid (1/1) (3.44 g, 4.71 mmol) in DCM 10 mL and N-ethylbis(isopropyl)amine (1.52 g, 11.8 mmol) at RT. The resulting reaction mixture was stirred for overnight at RT. The progress of reaction was monitor by TLC/ELSD. The reaction mixture was diluted with dichloromethane (50.0 mL) and washed with water (50.0 mL) and brine solution (50.0 mL). The organic layer was dried over anhydrous Na₂SO₄, filtered and concentrated. The crude was purified by flash column chromatography (SiO₂: 0-35% EtOAc in heptane) to give N-[3-(bis{2-[(tert-butyl)bis(methyl)siloxy]decyl}amino)propyl]({[N-3-(bis{2-[(tert-butyl)bis(methyl)siloxy]decyl}amino)propylcarbamoyl]methyl}-N-methylamino)acetamide [7](650 mg, 20.56% Yield) as a pale yellow liquid.

### Result:

ELSD analysis: Purity 98.39%, Calculated C₇₅H₁₆₁N₅O₆Si₄=1340.15, Observed=1341.05 (m/z, M+H+).

### Synthesis of Compound LXXV

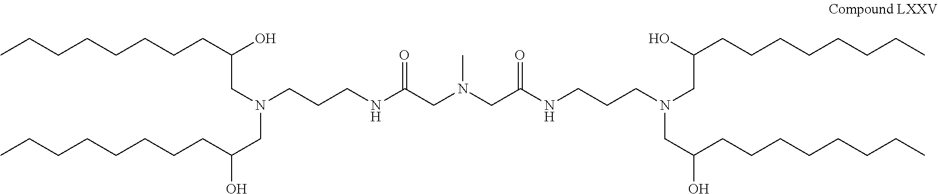 [SMILES: CCCCCCCCC(O)CN(CCCNC(=O)CN(C)CC(=O)NCCCN(CC(O)CCCCCCCC)CC(O)CCCCCCCC)CC(O)CCCCCCCC]

As depicted in Scheme 27: To a stirred solution of N-[3-(bis{2-[(tert-butyl)bis(methyl)siloxy]decyl}amino)propyl]({[N-3-(bis{2-[(tert-butyl)bis(methyl)siloxy]decyl}amino)propylcarbamoyl]methyl}-N-methylamino) acetamide [7](630 mg, 470 μmol) in tetrahydrofuran (7 mL) was added hydrogen fluoride-pyridine (1/1) (1.27 mL, 14.1 mmol) at 0° C. Reaction mixture was stirred for overnight at RT. The reaction progress was monitored by TLC/ELSD. After SM consumed, reaction mixture was quenched with aq sodium bicarbonate solution upto pH 8, and extracted with Ethyl acetate (2×50.0 mL). Organic layer was dried over anhy. sodium sulphate, filtered and concentrated under reduced pressure to get crude. The crude was taken in ACN (10.0 mL) and extracted with heptane (2×20.0 mL). The heptane layer was concentrated under vacuum to get N-{3-[bis(2-hydroxydecyl)amino]propyl}[({N-3-[bis(2-hydroxydecyl)amino]propylcarbamoyl}methyl)-N-methylamino]acetamide (230 mg, 55.37% Yield) as pale yellow liquid.

### Result:

¹H NMR (400 MHz, CDCl₃): δ 8.30-7.80 (brs, 2H), 3.95-3.65 (m, 4H), 3.52-3.36 (m, 4H), 3.17-3.06 (m, 4H), 3.00-2.68 (m, 7H), 2.63-2.28 (m, 8H), 2.04-1.65 (m, 4H), 1.65-1.0 (m, 60H), 0.89-0.80 (t, J=6.8 Hz, 12H) ppm.

ELSD analysis: Purity 99.84%, Calculated C₅₁H₁₀₅N₅O₆=883.81, Observed=884.75 (m/z, M+H⁺).

### Example 28: Synthesis of Compound CVII

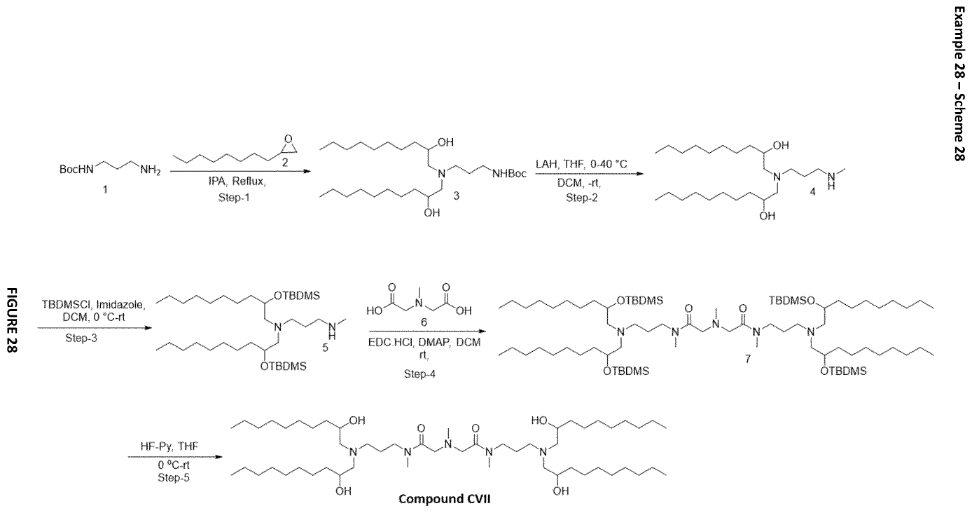

For example, the compounds of the invention may be prepared according to Scheme 28 (as depicted in FIG. 28).

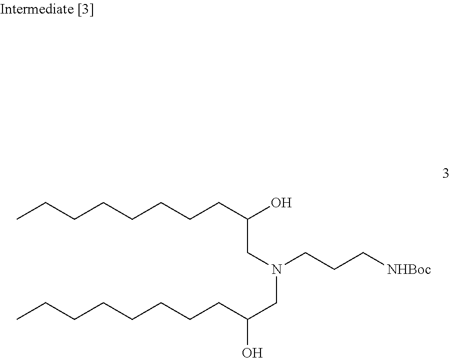 [SMILES: CCCCCCCCC(O)CN(CCCNC(=O)OC(C)(C)C)CC(O)CCCCCCCC]

As depicted in Scheme 28: To a stirred solution of tert-butyl (3-aminopropyl)carbamate [1](2.0 g, 11.5 mmol) and 2-octyloxirane [2](3.95 g, 25.3 mmol) in isopropanol (40 mL) was added ethylbis(propan-2-yl)amine (1.48 g, 11.5 mmol). The resultant reaction mixture was stirred for 16 h at 90° C. The reaction progress was monitored by TLC and ELSD. Starting material was consumed completely. Reaction mass was concentrated under reduced pressure to get crude compound, which was purified by silica gel flash column chromatography (0-5% MeOH in DCM) to give tert-butyl N-{3-[bis(2-hydroxydecyl)amino]propyl}carbamate [3](5.8 g, 93.4% yield) as a colourless liquid Result:

ELSD analysis: Purity 96.6%, Calculated C₂₈H₅₈N₂O₄=486.44, Observed=487.40 (m/z, M+H⁺).

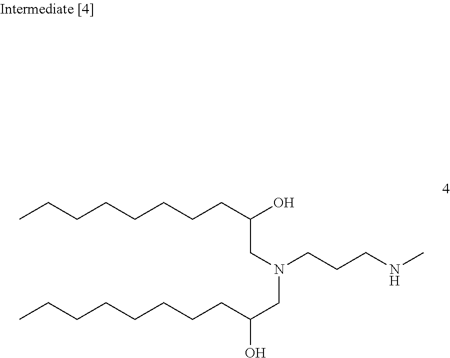 [SMILES: CCCCCCCCC(O)CN(CCCNC)CC(O)CCCCCCCC]

As depicted in Scheme 28: To a stirred solution of 3-[bis(2-hydroxydecyl)amino]propylamino-tert-butylformylate (6.98 g, 14.3 mmol) in tetrahydrofuran (100 mL) was added aluminium (3+) lithium tetrahydride (5.44 g, 10 eq., 143 mmol) at 0° C. The reaction mixture was stirred at RT for 48 hr. The progress of reaction was monitored by TLC and ELSD. After completion of reaction, reaction mixture was cooled to 0° C. and Fisher workup was done. The mixture was diluted with ethyl acetate and filtered by sintered funnel. The organic layer was separated, dried over Na₂SO₄, concentrated under reduced pressure to obtain 1-{(2-hydroxydecyl)[3-(methylamino)propyl]amino}-2-decanol (5.5 g, 95.73% Yield) as a colourless liquid, which was used for next step without further purification.

### Results:

ELSD analysis: Purity 99.55%, Calculated C₂₄H₅₂N₂O₂=400.40, Observed=401.45 (m/z, M+H⁺).

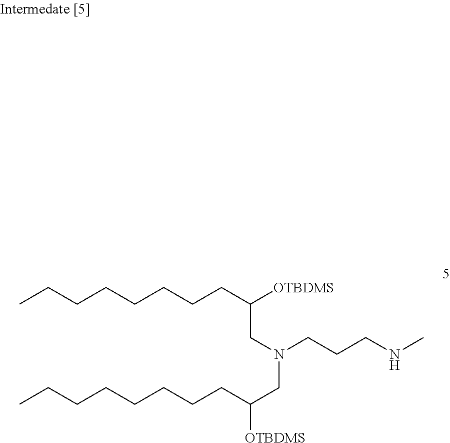 [SMILES: CCCCCCCCC(CN(CCCNC)CC(CCCCCCCC)O[Si](C)(C)C(C)(C)C)O[Si](C)(C)C(C)(C)C]

As depicted in Scheme 28: To a stirred solution of 1-{(2-hydroxydecyl)[3-(methylamino)propyl]amino}-2-decanol [4](5.5 g, 13.7 mmol) in dichloromethane (100 mL) was added imidazole (9.34 g, 137 mmol) followed by the addition of (tert-butyl)(chloro)bis(methyl)silane (12.4 g, 82.4 mmol). The reaction mixture was stirred at room temperature for 16 hr. The progress of reaction was monitored by ELSD/TLC. After completion of reaction, reaction mass was diluted with water (20 mL) and extracted with DCM (2×100 mL). The combined organic layer was dried over anhydrous sodium sulphate and concentrated under reduced pressure to get crude. The crude was purified by flash column chromatography (SiO₂: 0-10% MeOH/DCM) to get 1-(bis{2-[(tert-butyl)bis(methyl)siloxy]decyl}amino)-3-(methylamino)propane [5](4.3 g, 49.79 5 Yield) as a greenish viscous liquid.

### Results:

ELSD analysis: Purity 99.44%, Calculated C₃₆H₃₀N₂O₂Si₂=628.58, Observed=629.55 (m/z, M+H⁺).

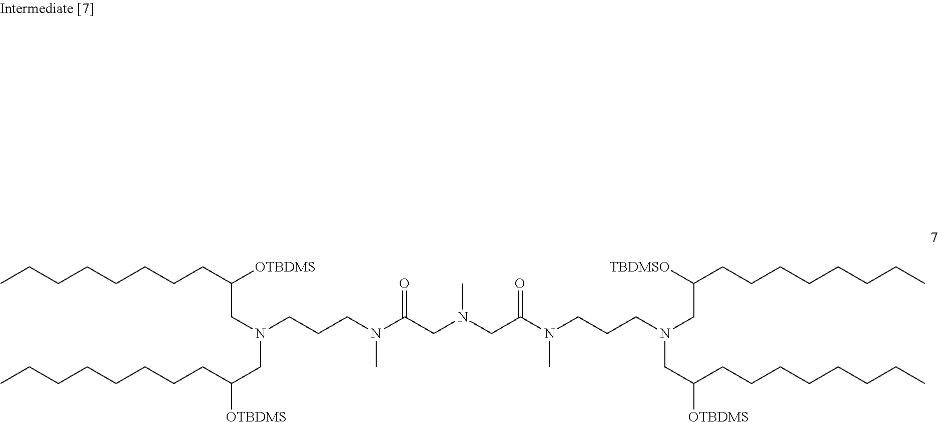 [SMILES: CCCCCCCCC(CN(CCCN(C)C(=O)CN(C)CC(=O)N(C)CCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 28: To a stirred solution of [(carboxymethyl)-N-methylamino]acetic acid [6](0.3 g, 2.04 mmol) in dimethylformamide (20.0 mL) was added N-ethylbis(isopropyl)amine (1.32 g, 10.2 mmol) followed by the addition of 1,1,3,3-tetramethyl-2-(3H-1,2,3,4-tetraazainden-3-yl)-3-isoureaium hexafluoridophosphate(1-) (1.55 g, 4.08 mmol). The reaction mixture was stirred for 10 min and then added 1-(bis{2-[(tert-butyl)bis(methyl) siloxy]decyl}amino)-3-(methylamino)propane [5](2.57 g, 4.08 mmol) to the reaction mixture. The reaction mixture was stirred at room temperature for 16 h. The progress of reaction was monitored by ELSD/TLC. After completion, reaction mass was quenched by water (10 mL) and extracted with ethyl acetate (3×20 mL). The organic layer was dried over sodium sulphate, concentrated under reduced pressure to get crude. The crude was purified by flash column chromatography (SiO₂: 0-30% ethyl acetate in hexane) to get 2,2′-(methylazanediyl)bis(N-(3-(bis(2-((tert-butyldimethylsilyl)oxy)decyl)amino)propyl)-N-methylacetamide) [7](0.4 g, 14.32% Yield) pale yellow liquid.

### Results:

ELSD analysis: Purity 99.35%, Calculated C₇₇H₁₆₅N₅O₆Si₄=1368.18, Observed=1369.0 (m/z, M+H⁺).

### Synthesis of Compound CVII

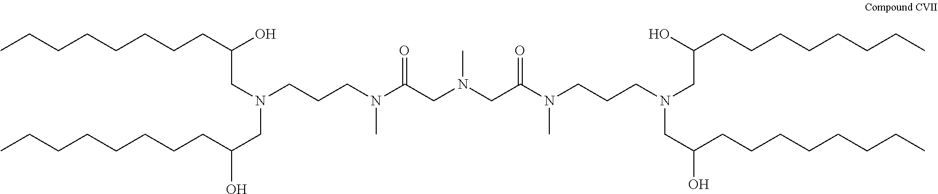 [SMILES: CCCCCCCCC(O)CN(CCCN(C)C(=O)CN(C)CC(=O)N(C)CCCN(CC(O)CCCCCCCC)CC(O)CCCCCCCC)CC(O)CCCCCCCC]

As depicted in Scheme 28: To a stirred solution of N-[3-(bis{2-[(tert-butyl)bis(methyl)siloxy]decyl}amino)propyl]-N-methyl[({[3-(bis{2-[(tert-butyl)bis(methyl)siloxy]decyl}amino)propyl]-N-methylcarbamoyl}methyl)-N-methylamino]acetamide (0.4 g, 292 μmol) in tetrahydrofuran (4.0 mL) was added hydrogen fluoride pyridine (1.4 mL, 15.5 mmol) at 0° C. under inert atmosphere. The resultant reaction mass was stirred at RT for 16 h. The progress of reaction was monitored by TLC. After completion of reaction, reaction mixture was quench by saturated sodium bicarbonate solution up to pH 8 and extracted with ethyl acetate (3×50 mL). The organic layer was dried over anhydride sodium sulphate, filtered and concentrated under reduced pressure to get crude. The crude was purified by flash column chromatography (SiO₂: 0-5% methanol in dichloromethane) to obtain N-{3-[bis(2-hydroxydecyl)amino]propyl}-N-methyl{[({3-[bis(2-hydroxydecyl)amino]propyl}-N-methylcarbamoyl)methyl]-N-methylamino}acetamide [Compound CVII](128 mg, 48.03% Yield) as a pale yellow liquid.

### Result:

¹H NMR (400 MHz, CDCl₃): δ 3.67-3.56 (m, 4H), 3.52-3.16 (m, 11H), 3.04-2.88 (m, 6H), 2.71-2.25 (m, 15H), 1.81-1.62 (m, 4H), 1.53-1.26 (m, 57H), 0.89-0.80 (t, J=6.8 Hz, 12H) ppm.

ELSD analysis: Purity 99.82%, Calculated C₅₃H₁₀₉N₅O₆=911.84, Observed=912.75 (m/z, M+H⁺).

### Example 29: Synthesis of Compound LXXVI

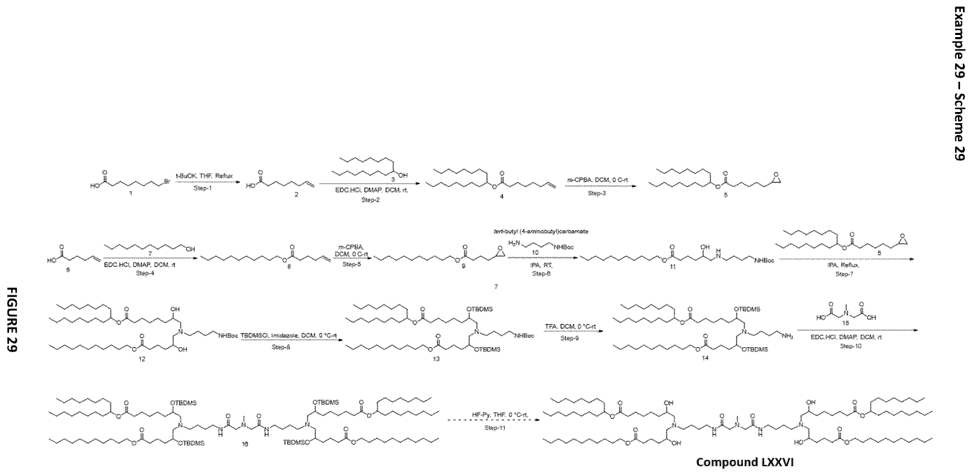

For example, the compounds of the invention may be prepared according to Scheme 29 (as depicted in FIG. 29).

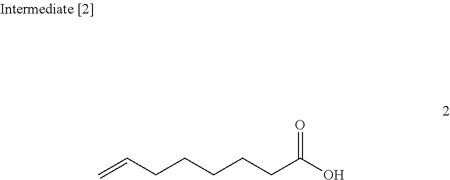

As depicted in Scheme 29: To a stirred solution of 8-bromooctanoic acid [1](100 g, 0.448 mol) in tetrahydrofuran (3.25 L) was added potassium 2-methylpropan-2-olate (226 g, 2.02 mol) at RT under nitrogen atmosphere. The reaction mixture was stirred at 90° C. for 16 h. The progress of reaction was monitored by TLC. After completion, reaction mixture 5 quenched by 2M HCl up to pH 3 and extracted by ethyl acetate (3×250 mL). The combined organic layer was dried over anhydrous sodium sulphate, filtered and evaporated under reduced pressure to get oct-7-enoic acid [2](61.0 g, 95.76% Yield) as a light yellow liquid.

### Result:

¹H-NMR (400 MHz, CDCl₃)-δ 11.96 (s, 1H), 5.83-5.73 (m, 1H), 5.01-4.92 (m, 2H), 2.20-2.16 (t, J=7.2 Hz, 2H), 2.03-1.98 (m, 2H), 1.54-1.45 (m, 2H), 1.38-1.30 (m, 2H), 1.30-1.23 (m, 2H).

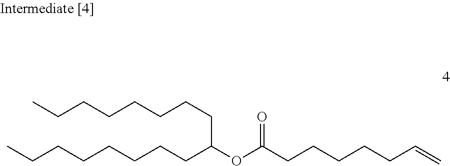 [SMILES: C=CCCCCCC(=O)OC(CCCCCCCC)CCCCCCCC]

As depicted in Scheme 29: In a dry RBF, to a stirred solution oct-7-enoic acid [2](30 g, 211 mmol) in dichloromethane (750 mL) was added EDC·HCl (50.6 g, 264 mmol) and N,N-dimethylpyridin-4-amine (6.7 g, 54.9 mmol). The reaction mixture was stirred for 15 min and then heptadecan-9-ol [3](48.7 g, 190 mmol) at room temperature under nitrogen atmosphere. The reaction mixture was stirred at RT for 18 h. The progress of reaction was monitored by TLC and ELSD. The reaction mixture was quenched with water (150.0 mL) and extracted with DCM (2×100.0 mL). The organic layer was dried over anhy. sodium sulphate, filtered and concentrated under vacuum to give heptadecan-9-yl oct-7-enoate [4](26.5 g, crude), which was forwarded to next step without purification.

### Result:

ELSD analysis: Purity 46.23%, Calculated C₂₅H₄₈O₂=380.37, Observed=381.40 (m/z, M+H+).

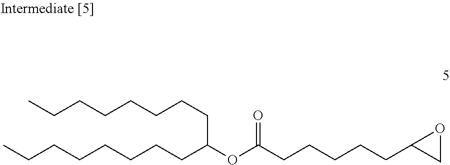 [SMILES: CCCCCCCCC(CCCCCCCC)OC(=O)CCCCCC1CO1]

As depicted in Scheme 29: To a stirred solution of heptadecan-9-yl oct-7-enoate [4](11 g, 28.9 mmol) in dichloromethane (200 L) was added 3-chlorobenzene-1-carboperoxoic acid (9.97 g, 57.8 mmol) at 0° C. under nitrogen atmosphere. The reaction mixture was stirred at RT for 16 hr. The reaction mixture was monitored by TLC. After completion, reaction mixture was quenched by saturated sodium bicarbonate and extracted with DCM (3×100 mL). The organic layer was dried over sodium sulphate and concentrated under reduced pressure to give crude. The crude product was again diluted with pentane and allow to stand for some time at low temperature. The solution was filtered and filtrate was concentrated under reduced pressure to give heptadecan-9-yl 6-(oxiran-2-yl)hexanoate [5](11.0 g, 96% Yield) as a colourless liquid.

### Results:

¹H NMR (400 MHz, CDCl3): δ 4.90-4.83 (m, 1H), 2.92-2.88 (m, 1H), 2.75-2.73 (t, J=4.4, 1H), 2.47-2.42 (m, 1H), 2.31-2.27 (t, J=7.6 Hz, 2H), 1.68-1.60 (m, 2H), 1.53-1.34 (m, 10H), 1.32-1.20 (m, 24H), 0.89-0.86 (t, J=6.8 Hz, 6H) ppm.

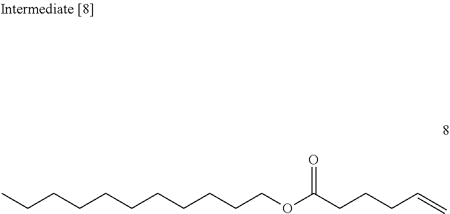 [SMILES: C=CCCCC(=O)OCCCCCCCCCCC]

As depicted in Scheme 29: To a stirred solution of hex-5-enoic acid [6](20.0 g, 175 mmol) in dichloromethane (300 mL), was added DMAP (5.61 g, 45.6 mmol) and EDC·HCl (40.3 g, 210 mmol). The reaction mixture was stirred for 15 min and then added undecan-1-ol [7](28.7 g, 166 mmol). The reaction mixture was stirred was at RT for 16 h. The progress of reaction was monitored by ELSD/TLC. The reaction mass was concentrated under reduced pressure to give crude. The resulting crude was purified by flash column chromatography (SiO₂: 2-5% ethyl acetate in n-heptane to get undecyl hex-5-enoate [8](43.0 g, 91.48% Yield) as a colourless liquid.

### Result:

¹H NMR (400 MHz, CDCl3): δ 5.83-5.76 (m, 1H), 5.06-4.99 (m, 2H), 4.09-4.05 (t, J=6.4 Hz, 2H), 2.33-2.30 (t, J=7.6 Hz, 2H), 2.13-2.05 (m, 2H), 1.78-1.70 (m, 2H), 1.66-1.59 (m, 2H), 1.39-1.20 (m, 16H), 0.90-0.88 (t, J=6.8 Hz, 3H) ppm.

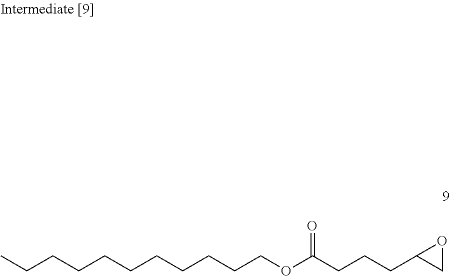 [SMILES: CCCCCCCCCCCOC(=O)CCCC1CO1]

As depicted in Scheme 29: To a stirred solution of undecyl hex-5-enoate [8](20.0 g, 74.5 mmol) in dichloromethane (200 mL) was added 3-chlorobenzene-1-carboperoxoic acid (30.9 g, 134 mmol) portion wise at 0° C. The reaction was stirred at RT for 16 h. The progress of reaction was monitored by TLC & ELSD. The reaction mixture was quenched by saturated sodium bicarbonate and extracted with DCM. The organic layer was dried over anhydrous sodium sulphate and concentrated under vacuum. The crude was purified by flash column chromatography (SiO₂: 5-15% EA:Heptane) undecyl 4-(oxiran-2-yl)butanoate [9](9.0 g, 42.47% Yield) as a colourless liquid.

### Result:

ELSD analysis: Purity 94.79%, Calculated C₁₇H₃₂O₃=284.24, Observed=285.35 (m/z, M+H⁺).

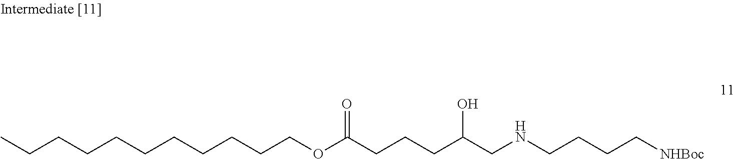 [SMILES: CCCCCCCCCCCOC(=O)CCCC(O)CNCCCCNC(=O)OC(C)(C)C]

As depicted in Scheme 29: To a stirred solution of tert-butyl (4-aminobutyl)carbamate [10](5.0 g, 26.6 mmol) in isopropanol (100 mL) was added undecyl 4-(2-oxiranyl)butyrate [9](7.55 g, 26.6 mmol). The reaction mixture was stirred at room temperature for 16 h. The progress of reaction was monitored by ELSD/TLC. After completion of reaction (starting material was consumed), the reaction mixture was concentrated under reduced pressure to get crude. The crude was purified over silica by using (0-10% MeOH/DCM) to obtain undecyl 6-[4-(tert-butoxycarbonylamino) butylamino]-5-hydroxyhexanoate [11](4.3 g, 34.25% Yield) as a colourless liquid.

### Result:

ELSD analysis: Purity 99.45%, Calculated C₂₆H₅₂N₂O₅=472.39, Observed=473.35 (m/z, M+H⁺).

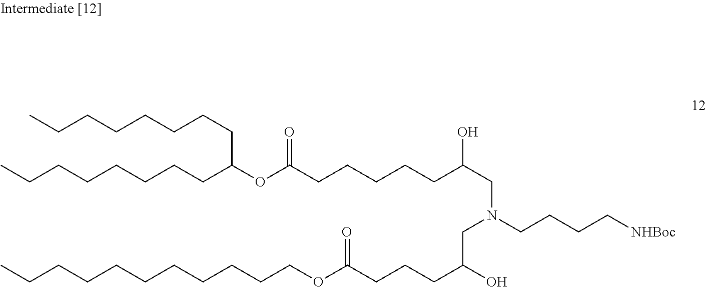 [SMILES: CCCCCCCCCCCOC(=O)CCCC(O)CN(CCCCNC(=O)OC(C)(C)C)CC(O)CCCCCC(=O)OC(CCCCCCCC)CCCCCCCC]

As depicted in Scheme 29: To a stirred solution of undecyl 6-[4-(tert-butoxycarbonylamino)butylamino]-5-hydroxyhexanoate [11](4.3 g, 9.1 mmol) in IPA (25 mL) was added 1-octylnonyl 6-(2-oxiranyl)hexanoate [5](3.97 g, 10 mmol). The reaction mixture was stirred at 90° C. for 16 h. The progress of reaction was monitored by TLC and ELSD. After completion of reaction, reaction mixture was concentrated under reduced pressure to get crude, which was purified by flash column chromatography (SiO₂: 0-10% MeOH in DCM) to give 1-octylnonyl 8-{[4-(tert-butoxycarbonylamino)butyl][2-hydroxy-5-(undecyloxycarbonyl)pentyl]amino}-7-hydroxyoctanoate [12](3.9 g, 49.32% Yield) as a greenish liquid.

### Result:

ELSD analysis: Purity 96.57%, Calculated C₅₁H₁₀₀N₂O₈=868.75, Observed=869.70 (m/z, M+H⁺).

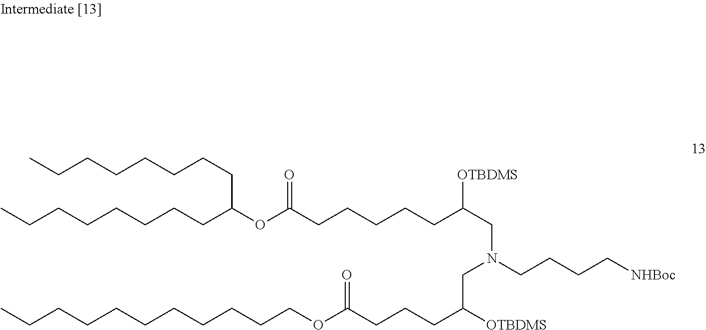 [SMILES: CCCCCCCCCCCOC(=O)CCCC(CN(CCCCNC(=O)OC(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 29: To a stirred solution of 1-octylnonyl 8-{[4-(tert-butoxycarbonylamino)butyl][2-hydroxy-5-(undecyloxycarbonyl)pentyl]amino}-7-hydroxyoctanoate [12](3.9 g, 4.49 mmol) in dichloromethane (30 mL) was added imidazole (3.05 g, 44.9 mmol) and (tert-butyl)(chloro)bis(methyl)silane (4.06 g, 26.9 mmol). The reaction mixture was stirred at RT for 16 hr. The progress of reaction was monitored by TLC and ELSD. After completion of reaction, the reaction mass was quenched with water (30 ml) and extracted with DCM (3×30 ml). The organic layer was collected, dried over sodium sulphate and concentrated under reduced pressure to get crude. The crude was purified over silica using 0-10% ethyl acetate in hexane to give desired product as 1-octylnonyl 8-{[4-(tert-butoxycarbonylamino) butyl]{2-[(tert-butyl)bis(methyl)siloxy]-5-(undecyloxycarbonyl)pentyl}amino}-7-[(tert-butyl)bis(methyl) siloxy]octanoate [13](3.2 g, 64.97% Yield) as a colourless liquid.

### Result:

ELSD analysis: Purity 98.03%, Calculated C₆₃H₁₂₈N₂O₈Si₂=1096.92, Observed=1097.80 (m/z, M+H⁺).

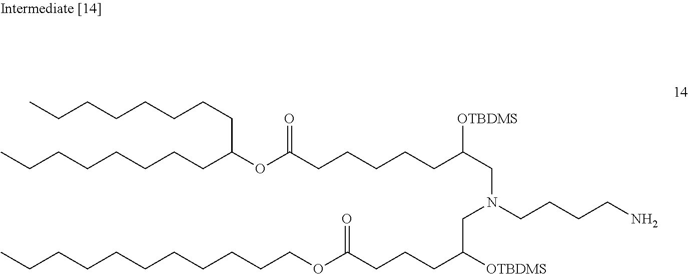 [SMILES: CCCCCCCCCCCOC(=O)CCCC(CN(CCCCN)CC(CCCCCC(=O)OC(CCCCCCCC)CCCCCCCC)O[Si](C)(C)C(C)(C)C)O[Si](C)(C)C(C)(C)C]

As depicted in Scheme 29: To a stirred solution of 1-octylnonyl 8-{[4-(tert-butoxycarbonylamino)butyl]{2-[(tert-butyl)bis(methyl)siloxy]-5-(undecyloxycarbonyl)pentyl}amino}-7-[(tert-butyl)bis(methyl) siloxy]octanoate [13](2.8 g, 2.55 mmol) in dichloromethane (20 mL) was added trifluoroacetic acid (1.95 mL, 10 eq., 25.5 mmol) at 0° C. The reaction mixture was stirred at RT for 3 hr. The progress of reaction was monitored by ELSD. After completion of reaction, the reaction mass was concentrated under reduced pressure to get desired compound [14](2.54 g, Crude), which was used as such for next step.

### Result:

ELSD analysis: Purity 99.82%, Calculated C₅₈H₁₂₀N₂O₆Si₂=996.87, Observed=997.75 (m/z, M+H⁺).

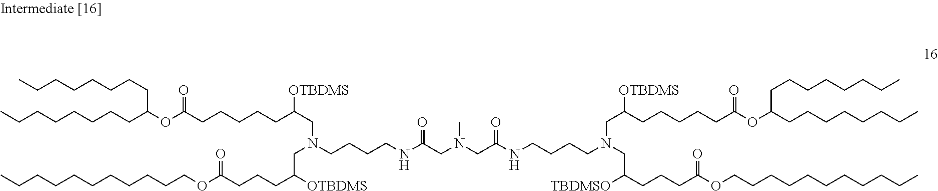 [SMILES: CCCCCCCCCCCOC(=O)CCCC(CN(CCCCNC(=O)CN(C)CC(=O)NCCCCN(CC(CCCCCC(=O)OC(CCCCCCCC)CCCCCCCC)O[Si](C)(C)C(C)(C)C)CC(CCCC(=O)OCCCCCCCCCCC)O[Si](C)(C)C(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 29: To a stirred solution of [(carboxymethyl)-N-methylamino]acetic acid [15](160 mg, 1.09 mmol) in dichloromethane (20 mL) was added N,N-dimethyl-4-pyridylamine (399 mg, 3.26 mmol) and EDC·HCl (625 mg, 3.26 mmol) at room temperature. The reaction mixture was stirred for 15 min at RT and then added 1-octylnonyl 8-[(4-aminobutyl){2-[(tert-butyl)bis(methyl)siloxy]-5-(undecyloxycarbonyl)pentyl}amino]-7-[(tert-butyl)bis(methyl) siloxy]octanoate [14](2.39 g, 2.39 mmol). The reaction mixture was stirred for 16 h. The progress of reaction was monitored by TLC. After completion, reaction mixture was diluted with brine solution and extracted with DCM. The organic layers were combined, dried over sodium sulphate, concentrated under reduced pressure to get crude. The crude was purified by flash column chromatography (SiO₂: 0-40% Ethyl acetate in n-hexane) to afford 1-octylnonyl 8-[(4-{[({[N-4-({2-[(tert-butyl)bis(methyl)siloxy]-7-(1-octylnonyloxycarbonyl)heptyl}{2-[(tert-butyl)bis(methyl)siloxy]-5-(undecyloxycarbonyl)pentyl}amino)butylcarbamoyl]methyl}-N-methylamino)methyl]carbonylamino}butyl){2-[(tert-butyl)bis(methyl)siloxy]-5-(undecyloxycarbonyl)pentyl}amino]-7-[(tert-butyl)bis(methyl)siloxy]octanoate [16](1.6 g, 69.84% Yield) as a pale yellow.

### Result:

ELSD analysis: Purity 98.42%, Calculated C₁₂₁H₂₄₅N₅O₁₄Si₄=2104.77, Observed=1053.90 (m/z, M/2+H⁺).

### Synthesis of Compound LXXVI

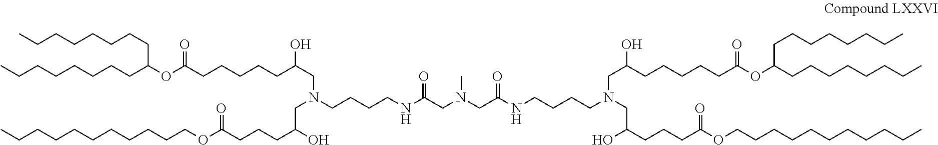 [SMILES: CCCCCCCCCCCOC(=O)CCCC(O)CN(CCCCNC(=O)CN(C)CC(=O)NCCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC]

As depicted in Scheme 29: To a stirred solution of 1-octylnonyl 8-[(4-{[({[N-4-({2-[(tert-butyl)bis(methyl)siloxy]-7-(1-octylnonyloxycarbonyl)heptyl}{2-[(tert-butyl)bis(methyl)siloxy]-5-(undecyloxycarbonyl) pentyl}amino)butylcarbamoyl]methyl}-N-methylamino)methyl]carbonylamino}butyl){2-[(tert-butyl)bis(methyl)siloxy]-5-(undecyloxycarbonyl)pentyl}amino]-7-[(tert-butyl)bis(methyl)siloxy]octanoate [16](0.5 g, 237 μmol) in tetrahydrofuran (5 mL) was added hydrogen fluoride pyridine (978 μL, 7.59 mmol) at 0° C. under inert atmosphere. The resultant reaction mass was allowed to stir at RT for 16 h. The progress of reaction was monitored by TLC. After completion of reaction, reaction mixture was quench by saturated sodium bicarbonate solution (up to pH 8) and extracted with ethyl acetate (3×50 mL). The organic layer was dried over anhydride sodium sulphate, filtered and concentrated under reduced pressure to get crude. The crude was purified by flash column chromatography (SiO₂: 0-5% methanol in DCM) to give 1-octylnonyl 8-({4-[({[(N-4-{[2-hydroxy-7-(1-octylnonyloxycarbonyl) heptyl][2-hydroxy-5-(undecyloxycarbonyl)pentyl]amino}butylcarbamoyl)methyl]-N-methylamino}methyl) carbonylamino]butyl}[2-hydroxy-5-(undecyloxycarbonyl)pentyl]amino)-7-hydroxyoctanoate [Compound LXXVI](175 mg, 44.70% Yield) as a pale yellow liquid.

### Result:

¹H NMR (400 MHz, CDCl₃): δ 7.48-7.18 (brs, 2H), 4.96-4.77 (m, 2H), 4.01-3.97 (t, J=6.8 Hz, 4H), 3.68-3.52 (m, 4H), 3.31-3.19 (m, 4H), 3.05 (s, 4H), 2.68-2.45 (m, 8H), 2.43-2.34 (m, 4H), 2.32-2.19 (m, 13H), 1.81-1.62 (m, 4H), 1.61-1.50 (m, 12H), 1.49-1.39 (m, 12H), 1.37-1.13 (m, 96H), 0.89-0.81 (t, J=6.8 Hz, 18H).

ELSD analysis: Purity 99.85%, Calculated C₉₇H₁₈₉N₅O₁₄=1648.42, Observed=1649.20 (m/z, M+H+).

### Example 30: Synthesis of Compound LXXVII

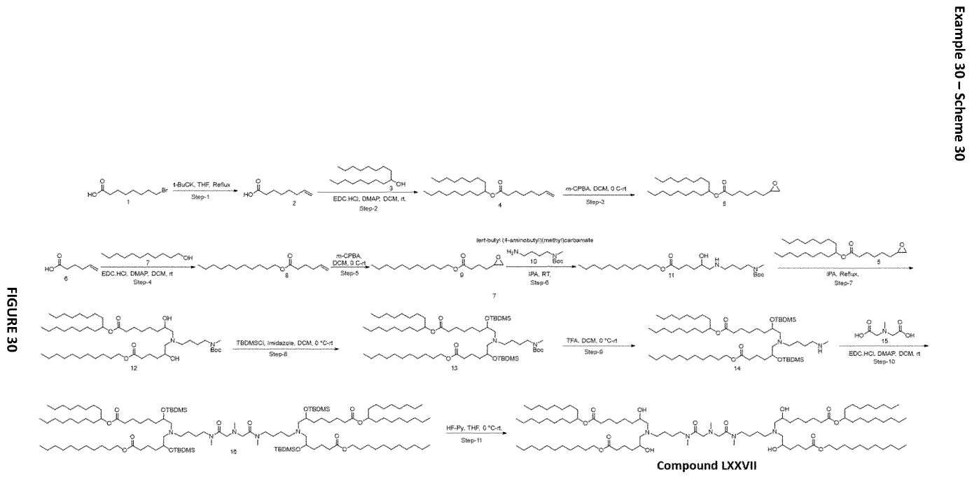

For example, the compounds of the invention may be prepared according to Scheme 30 (as depicted in FIG. 30).

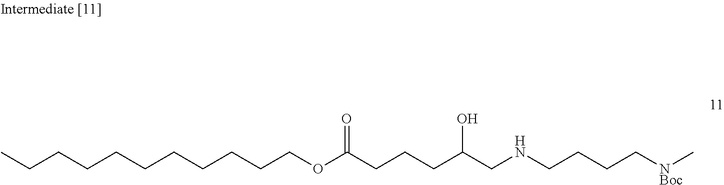 [SMILES: CCCCCCCCCCCOC(=O)CCCC(O)CNCCCCN(C)C(=O)OC(C)(C)C]

As depicted in Scheme 30: To a stirred solution tert-butyl (4-aminobutyl)(methyl)carbamate [10](4 g, 19.8 mmol) in IPA (100 mL) was added undecyl 4-(2-oxiranyl)butyrate [9](5.06 g, 17.8 mmol). The reaction mixture was stirred for 32 hr at RT. The progress of reaction was monitored by TLC. After completion of reaction, reaction mixture was concentrated under reduced pressure to get crude. The crude was purified over silica using 0-20% methanol in DCM to give undecyl 6-[4-(N-tert-butoxycarbonyl-N-methylamino)butylamino]-5-hydroxyhexanoate [11](2.8 g, 25.31% Yield) as colourless solid.

### Result:

ELSD analysis: Purity 87.81%, Calculated C₂₇H₅₄N₂O₅=486.40, Observed=487.40 (m/z, M+H⁺).

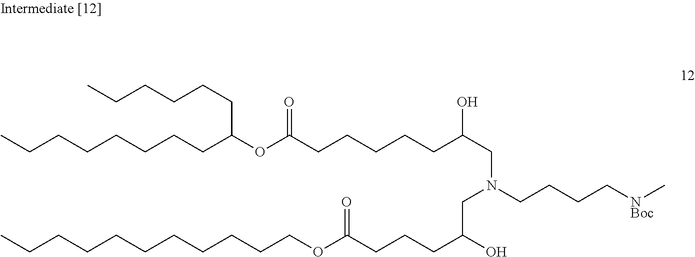 [SMILES: CCCCCCCCCCCOC(=O)CCCC(O)CN(CCCCN(C)C(=O)OC(C)(C)C)CC(O)CCCCCC(=O)OC(CCCCCC)CCCCCCCC]

As depicted in Scheme 30: To a stirred solution of undecyl 6-[4-(N-tert-butoxycarbonyl-N-methylamino)butylamino]-5-hydroxyhexanoate [11](2.88 g, 5.92 mmol) in isopropanol (60 mL) was added 1-octylnonyl 6-(2-oxiranyl)hexanoate [5](2.58 g, 6.52 mmol) at RT. The reaction mixture was heated at 90° C. for 16 h. The progress of reaction was monitored by TLC/ELSD. After completion of reaction, reaction mixture was concentrated under reduced pressure to get crude, which was purified by flash column chromatography (SiO₂: 0-5% MeOH:DCM) to obtain 1-octylnonyl8-{[4-(N-tert-butoxycarbonyl-N-methylamino)butyl][2-hydroxy-5-(undecyloxycarbonyl) pentyl]amino}-7-hydroxyoctanoate [12] (3.1 g, 59.24% Yield) as a pale yellow liquid.

### Result:

ELSD analysis: Purity 53.33%, Calculated C₅₂H₁₀₂N₂O₈=882.76, Observed=883.40 (m/z, M+H⁺).

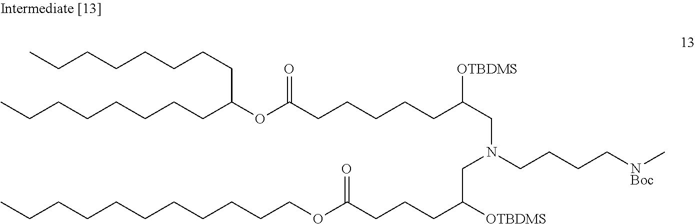 [SMILES: CCCCCCCCCCCOC(=O)CCCC(CN(CCCCN(C)C(=O)OC(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 30: To a stirred solution of 1-octylnonyl 8-{[4-(N-tert-butoxycarbonyl-N-methylamino)butyl][2-hydroxy-5-(undecyloxycarbonyl)pentyl]amino}-7-hydroxyoctanoate [12](3.1 g, 3.51 mmol), in dichloromethane (150 mL) was added imidazole (2.39 g, 35.1 mmol) followed by the addition of (tert-butyl)(chloro)bis(methyl)silane (4.23 g, 28.1 mmol). The reaction mixture was stirred at room temperature for 16 h. The progress of reaction was monitored by ELSD/TLC. After completion, reaction mass diluted with water (30 mL) and extracted with DCM (2×100 mL). The combined organic layer was dried over anhydrous sodium sulphate and concentrated under reduced pressure to get crude. The crude was then purified by flash column chromatography (SiO₂: 0-20% EtOAc/Heptane) to get 1-octylnonyl 8-{[4-(N-tert-butoxycarbonyl-N-methylamino)butyl]{2-[(tert-butyl)bis(methyl)siloxy]-5-(undecyloxycarbonyl)pentyl}amino}-7-[(tert-butyl)bis(methyl)siloxy]octanoate [13](1.0 g, 25.63% Yield) as pale yellow liquid.

### Result:

ELSD analysis: Purity 98.03%, Calculated C₆₄H₁₃₀N₂O₈Si₂=1110.94, Observed=1111.40 (m/z, M+H⁺).

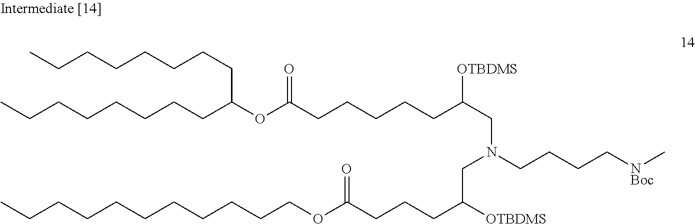 [SMILES: CCCCCCCCCCCOC(=O)CCCC(CN(CCCCN(C)C(=O)OC(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 30: To a stirred solution of 1-octylnonyl 8-{[4-(N-tert-butoxycarbonyl-N-methylamino)butyl]{2-[(tert-butyl)bis(methyl)siloxy]-5-(undecyloxycarbonyl)pentyl}amino}-7-[(tert-butyl)bis(methyl)siloxy]octanoate [13](1 g, 899 μmol) in dichloromethane (10 mL) was added trifluoroacetic acid (1.03 mL, 13.5 mmol) at 0° C. The reaction mixture was stirred at room temperature for 5 h. The progress of reaction was monitored by TLC/ELSD. After completion of reaction, reaction mixture was quenched with saturated sodium bicarbonate upto pH 8. The solution was extracted with DCM (2×50 mL). The organic layer was dried over anhy. sodium sulphate and concentrated under reduced pressure to get heptadecan-9-yl 7-((tert-butyldimethylsilyl)oxy)-8-((2-((tert-butyldimethylsilyl)oxy)-6-oxo-6-(undecyloxy)hexyl)(4-(methylamino)butyl)amino)octanoate [14] (0.9 g, crude) as a pale yellow liquid, which was used as such for next step without purification.

### Result:

ELSD analysis: Purity 96.13%, Calculated C₅₉H₁₂₂N₂O₆Si₂=1010.88, Observed=1011.50 (m/z, M+H⁺).

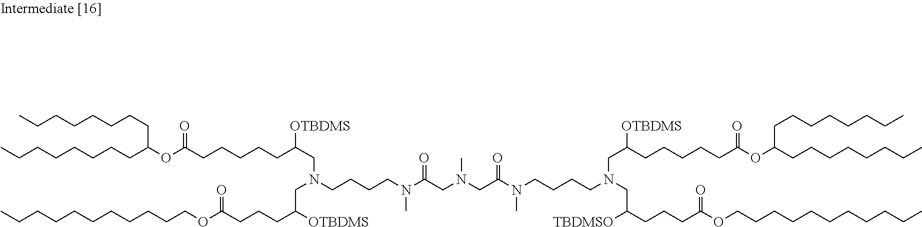 [SMILES: CCCCCCCCCCCOC(=O)CCCC(CN(CCCCN(C)C(=O)CN(C)CC(=O)N(C)CCCCN(CC(CCCCCC(=O)OC(CCCCCCCC)CCCCCCCC)O[Si](C)(C)C(C)(C)C)CC(CCCC(=O)OCCCCCCCCCCC)O[Si](C)(C)C(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 30: To a stirred solution of [(carboxymethyl)-N-methylamino]acetic acid [15](55 mg, 374 μmol) in dimethylformamide (10 mL) was added N-ethylbis(isopropyl)amine (242 mg, 1.87 mmol) and 1,1,3,3-tetramethyl-2-(3H-1,2,3,4-tetraazainden-3-yl)-3-isoureaium hexafluoridophosphate(1-) (355 mg, 935 μmol) at RT. The reaction mixture was stirred for 10 min at RT and added 1-octylnonyl 7-[(tert-butyl)bis(methyl)siloxy]-8-({2-[(tert-butyl)bis(methyl)siloxy]-5-(undecyloxycarbonyl)pentyl}[4-(methylamino)butyl]amino) octanoate [14](794 mg, 785 μmol). The reaction mixture was stirred at 60° C. for 16 h. The progress of reaction was monitored by TLC and ELSD. After completion of reaction, reaction mass was quenched by cold brine solution (10 ml) and extracted with ethyl acetate (3×15 mL). The organic layer was dried over sodium sulphate and concentrated under reduced pressure to get crude. The crude was purified by flash column chromatography (SiO₂: 0-10% ethyl acetate in heptane) to give di(heptadecan-9-yl) 7,27-bis((tert-butyldimethylsilyl)oxy)-9,25-bis(2-((tert-butyldimethylsilyl)oxy)-6-oxo-6-(undecyloxy)hexyl)-14,17,20-trimethyl-15,19-dioxo-9,14,17,20,25-pentaazatritriacontanedioate [16](210 mg, 26.32% Yield) as pale yellow liquid.

### Result:

ELSD analysis: Purity 99.37%, Calculated C₁₂₃H₂₄₉N₅O₁₄Si₄=2132.80, Observed=1067.30 (m/z, M/2+H⁺).

### Synthesis of Compound LXXVII

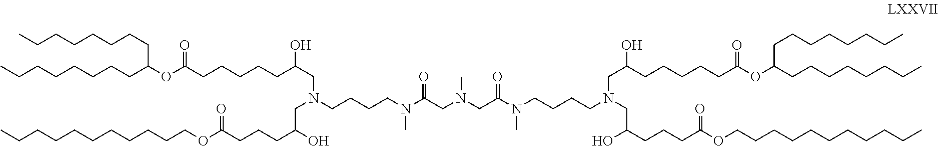 [SMILES: CCCCCCCCCCCOC(=O)CCCC(O)CN(CCCCN(C)C(=O)CN(C)CC(=O)N(C)CCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC]

As depicted in Scheme 30: To a stirred solution of di(heptadecan-9-yl) 7,27-bis((tert-butyldimethylsilyl)oxy)-9,25-bis(2-((tert-butyldimethylsilyl)oxy)-6-oxo-6-(undecyloxy)hexyl)-14,17,20-trimethyl-15,19-dioxo-9,14,17,20,25-pentaazatritriacontanedioate [16](0.2 g, 93.7 μmol) in tetrahydrofuran (2 mL) was added hydrogen fluoride pyridine (203 μL, 2.25 mmol) at 0° C. The reaction mixture was stirred for 16 h at room temperature. The progress of reaction was monitored by TLC. After completion of reaction, reaction mixture was quenched with cold saturated sodium bicarbonate solution up to pH 8 and extracted with ethyl acetate (3×10 mL). The organic layer was dried over sodium sulphate, concentrate under reduced pressure to get crude, which was purified by flash column chromatography (SiO₂: 0-10% methanol in DCM) to obtain di(heptadecan-9-yl) 7,27-dihydroxy-9,25-bis(2-hydroxy-6-oxo-6-(undecyloxy)hexyl)-14,17,20-trimethyl-15,19-dioxo-9,14,17,20,25-pentaazatritriacontanedioate (65 mg, 41.35% Yield) as pale yellow solid.

### Result:

¹H NMR (400 MHz, CDCl₃): δ 4.89-4.85 (m, 2H), 4.11-4.04 (t, J=6.8 Hz, 4H), 3.92-3.11 (m, 12H), 3.08-2.88 (m, 6H), 2.80-2.51 (brs, 6H), 2.50-2.43 (m, 4H), 2.37-2.33 (m, 4H), 2.32-2.28 (m, 4H), 2.08-1.88 (m, 3H), 1.86-1.78 (m, 3H), 1.76-1.56 (m, 14H), 1.55-1.48 (m, 12H), 1.46-1.21 (m, 94H), 1.20-0.97 (br 4H), 0.89-0.81 (t, J=6.8 Hz, 18H).

ELSD analysis: Purity 99.37%, Calculated C₉₉H₁₉₃N₅O₁₄=1676.45, Observed=1678.60 (m/z, M+H⁺).

### Example 31: Synthesis of Compound LXXVIII

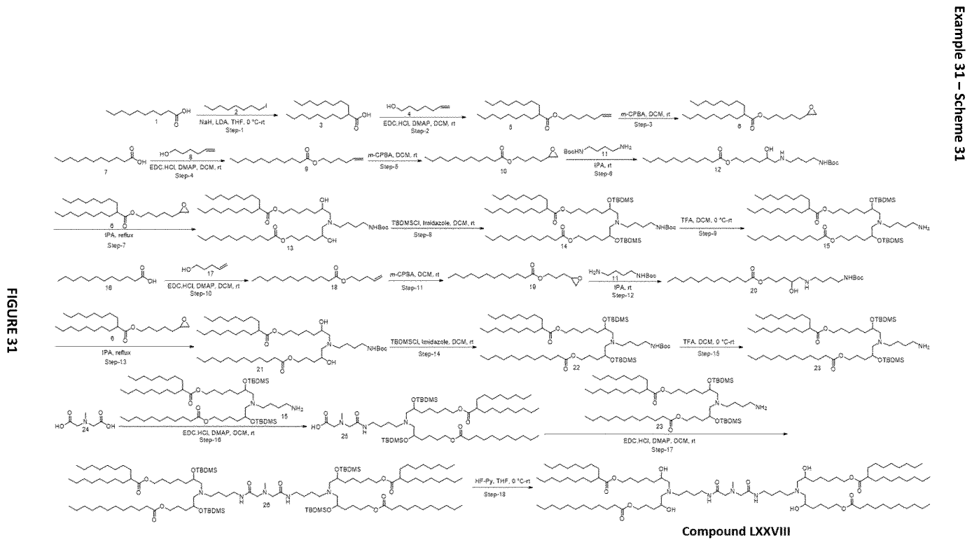

For example, the compounds of the invention may be prepared according to Scheme 31 (as depicted in FIG. 31).

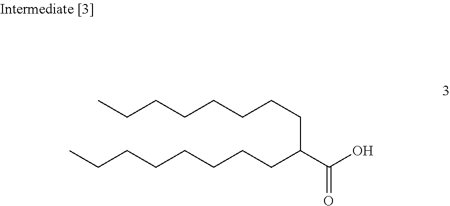 [SMILES: CCCCCCCCC(CCCCCCCC)C(=O)O]

As depicted in Scheme 31: To a stirred solution of decanoic acid [1](28 g, 163 mmol) in THF (0.5 L) was added sodium hydride (4.68 g, 195 mmol) at 0° C. Stirred for 15 minute, Then added lithium bis(isopropyl)azanide (20.9 g, 195 mmol) as drop wise at same temp. Reaction mass again stirred for 30 minute and then added 1-iodooctane [2](46.8 g, 195 mmol). Reaction mass was heated to 45° C. for next 6 h. Progress of reaction mass was monitored by ELSD and TLC. After completion, 1N HCl—50 ml was added into reaction mass and adjusted pH acidic. Reaction mass extracted with ethyl acetate-100 mL. Organic layer was dried over anhy. sodium sulphate, filter and concentrated under reduced pressure to get crude. The crude was purified by flash column chromatography (SiO₂: 0-3% ethyl acetate in n-hexane) to obtained 2-octyldecanoic acid [3](20 g, 43.25% Yield) as yellow liquid.

### Result:

¹H NMR (400 MHz, CDCl₃): δ 10.73 (s, 1H), 2.42-2.34 (m, 1H), 1.66-1.59 (m, 2H), 1.54-1.42 (m, 2H), 1.40-1.12 (m, 24H), 0.89-0.86 (t, J=6.8 Hz, 6H) ppm.

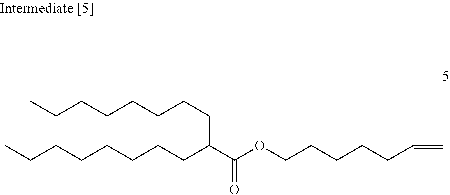 [SMILES: C=CCCCCCOC(=O)C(CCCCCCCC)CCCCCCCC]

As depicted in Scheme 31: To a stirred solution of 2-octyldecanoic acid [3](12.5 g, 43.8 mmol) in dichloromethane (0.2 L, 3.12 mol) were added N,N-dimethyl-4-pyridylamine (2.67 g, 21.9 mmol) and EDC·HCl (12.6 g, 65.7 mmol) followed by 6-hepten-1-ol [4](5.0 g, 43.8 mmol) at RT allowed to stir for 16 h. The progress of reaction was monitored by TLC (SM was consumed). After completion, water (50 mL) was added to the reaction mixture, and extract with DCM (2×200 mL). The resulting organic layer was dried over Na₂SO₄, and concentrated under reduce pressure to get crude. The crude was purified by flash column chromatography (SiO₂: 0-10% Ethyl acetate in n-heptane), to obtain 6-heptenyl 2-octyldecanoate [5](14.9 g, 89.39% Yield) as colourless liquid.

### Result:

¹H NMR (400 MHz, CDCl₃): δ 5.85-5.75 (m, 1H), 5.02-4.93 (m, 2H), 4.08-4.05 (t, J=6.8 Hz, 2H), 2.43-2.26 (m, 1H), 2.12-2.03 (m, 2H), 1.69-1.47 (m, 4H), 1.46-1.32 (m, 6H), 1.32-1.8 (m, 24H), 0.89-0.86 (t, J=6.8 Hz, 6H).

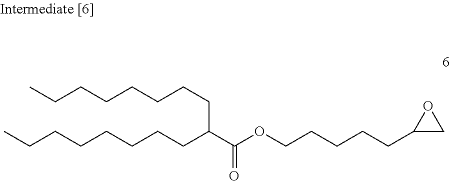 [SMILES: CCCCCCCCC(CCCCCCCC)C(=O)OCCCCCC1CO1]

As depicted in Scheme 31: To a stirred solution of 6-heptenyl 2-octyldecanoate [5](14.9 g, 39.1 mmol) in dichloromethane (0.3 L), was added m-chlorobenzeneperoxycarboxylic acid (14.9 g, 86.1 mmol) at 0° C., allowed to stir for 36 h at room temperature. The progress of reaction was monitored by TLC (SM was consumed). The reaction mixture was washed with cold aqueous sodium bicarbonate solution (200 mL). The resulting organic layer was dried over Na₂SO₄, and concentrated under reduce pressure, and the crude was purified by flash column chromatography (SiO2: 0-5% ethyl acetate in hexane), to give 5-(2-oxiranyl)pentyl 2-octyldecanoate [6](8.5 g, 54.74% Yield) as colourless liquid.

### Result:

¹H NMR (400 MHz, CDCl₃): δ 4.08-4.05 (t, J=6.8 Hz, 2H), 2.92-2.88 (m, 1H), 2.76-2.74 (t, J=4.4 Hz, 1H), 2.47-2.45 (m, 1H), 2.36-2.27 (m, 1H), 1.68-1.39 (m, 12H), 1.32-1.18 (m, 24H), 0.89-0.86 (t, J=6.8 Hz, 6H).

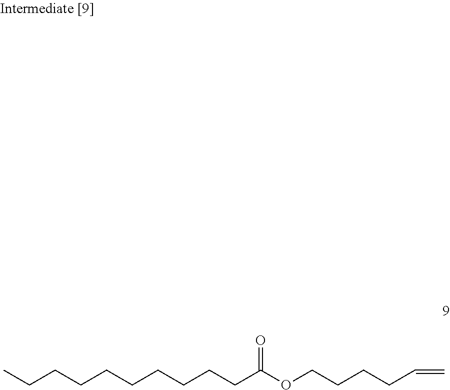 [SMILES: C=CCCCCOC(=O)CCCCCCCCCC]

As depicted in Scheme 31: To a stirred solution of undecanoic acid [7](51.2 g, 300 mmol) in dichloromethane (1.0 L, 9.37 mol), were added N,N-dimethyl-4-pyridylamine (15.24 g, 124.8 mmol) and EDC·HCl (71.8 g, 374 mmol) followed by 5-hexen-1-ol [8](25.0 g, 250 mmol) at room temperature. Reaction mass was allowed to stirred for 16 h at room temperature. The progress of reaction was monitored by ELSD/TLC (SM was consumed). Water (300 ml) was added to the reaction mixture and extract with DCM (2×500 ml). The separated organic layer was collect, combined, dried over anhy. Na₂SO₄ and concentrated under reduce pressure to obtain crude product. The crude was purified by flash column chromatography (SiO₂:0-5% EtOAc/Heptane) to obtain 5-hexenyl undecanoate [9](36.80 g, 54.9% Yield) as a colourless liquid.

### Result:

¹H NMR (400 MHz, CDCl₃): δ 5.84-5.74 (m, 1H), 5.03-4.94 (m, 2H), 4.08-4.04 (t, J=6.8 Hz, 2H), 2.32-2.25 (m, 2H), 2.10-2.05 (m, 2H), 1.70-1.54 (m, 4H), 1.51-1.41 (m, 2H), 1.37-1.10 (m, 14H), 0.89-0.86 (t, J=6.8 Hz, 3H).

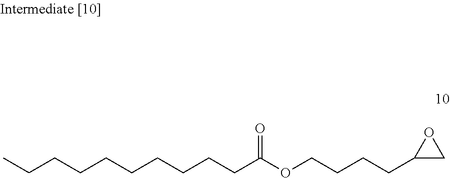 [SMILES: CCCCCCCCCCC(=O)OCCCCC1CO1]

As depicted in Scheme 31: To the stirred solution of 5-hexenyl undecanoate [9](33.5 g, 125 mmol) in dichloromethane (335 mL) at room temperature. Then was added 3-chlorobenzene-1-carboperoxoic acid (43.1 g, 187 mmol) at 0° C., allowed to stir for 16 h at room temperature. The progress of reaction was monitored by TLC (SM was consumed). The reaction mixture was washed with cold aqueous sodium bicarbonate solution (200 mL). The resulting organic layer was dried over Na2SO4, and concentrated under reduce pressure, and the crude was purified by flash column chromatography (SiO2: 0-5% ethyl acetate in hexane), to give 4-(2-oxiranyl)butyl undecanoate [10](15 g, 42.25% Yield) as a colourless liquid.

### Result:

¹H NMR (400 MHz, CDCl₃): δ 4.09-4.06 (t, J=6.8 Hz, 2H), 2.92-2.90 (m, 1H), 2.89-2.74 (m, 1H), 2.48-2.46 (m, 2H), 2.30-2.27 (t, J=6.8 Hz, 2H), 1.70-1.47 (m, 7H), 1.29-1.15 (m, 14H), 0.89-0.86 (t, J=6.8 Hz, 3H).

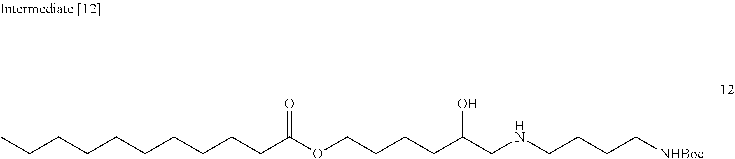 [SMILES: CCCCCCCCCCC(=O)OCCCCC(O)CNCCCCNC(=O)OC(C)(C)C]

As depicted in Scheme 31: To a stirred solution tert-butyl (4-aminobutyl)carbamate (14.7 g, 78.1 mmol) of in IPA (0.3 L) added 4-(2-oxiranyl)butyl undecanoate [10](20.0 g, 70.3 mmol) and allow to stirred reaction mix. to 32 hr at RT. Reaction progress was monitored by TLC. After completion, reaction mix. evaporate under reduced pressure to get crude. The crude was purified by flash column chromatography (SiO₂: 0-20% methanol in DCM) to give 6-[4-(tert-butoxycarbonylamino)butylamino]-5-hydroxyhexyl undecanoate [12](11.3 g 30.59% Yield) as white solid.

### Result:

ELSD analysis: Purity 99.54%, Calculated C₂₆H₅₂N₂O₅=472.39, Observed=473.40 (m/z, M+H⁺).

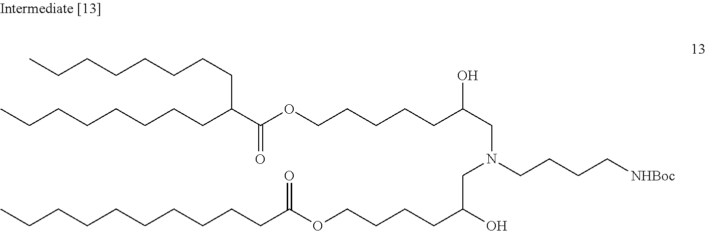 [SMILES: CCCCCCCCCCC(=O)OCCCCC(O)CN(CCCCNC(=O)OC(C)(C)C)CC(O)CCCCCOC(=O)C(CCCCCCCC)CCCCCCCC]

As depicted in Scheme 31: To a stirred solution of 6-[4-(tert-butoxycarbonylamino)butylamino]-5-hydroxyhexyl undecanoate [12](6 g, 12.7 mmol) in IPA (0.1 L) added N-ethylbis(isopropyl)amine (8.2 g, 63.5 mmol) and 5-(2-oxiranyl)pentyl 2-octyldecanoate [6](4.53 g, 11.4 mmol), allow to stirred at 90° C. for 16 h. The progress of reaction was monitored by ELSD data and TLC. After completion, reaction mass evaporated under reduced pressure to get crude. The crude was purified by flash column chromatography (SiO₂: 0-10% methanol in DCM) to get 6-{[4-(tert-butoxycarbonylamino)butyl][2-hydroxy-7-(1-octylnonylcarbonyloxy)heptyl]amino}-5-hydroxyhexyl undecanoate [13](6.2 g, 56.18 Yield) as pale yellow liquid.

### Result:

ELSD analysis: Purity 99.23%, Calculated C₅₁H₁₀₀N₂O₈=868.75, Observed=869.50 (m/z, M+H⁺).

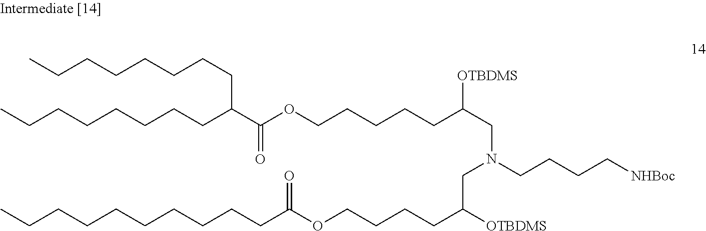 [SMILES: CCCCCCCCCCC(=O)OCCCCC(CN(CCCCNC(=O)OC(C)(C)C)CC(CCCCCOC(=O)C(CCCCCCCC)CCCCCCCC)O[Si](C)(C)C(C)(C)C)O[Si](C)(C)C(C)(C)C]

As depicted in Scheme 31: To a stirred solution of 6-{[4-(tert-butoxycarbonylamino)butyl][2-hydroxy-7-(1-octylnonylcarbonyloxy)heptyl]amino}-5-hydroxyhexyl undecanoate [13](6.2 g, 7.13 mmol) in dichloromethane (0.2 L) added imidazole (3.88 g, 57.1 mmol) and (tert-butyl)(chloro)bis(methyl)silane (10.7 g, 71.3 mmol) and allow to stirred at RT for 16 hr. The progress of reaction was monitored by TLC and ELSD data. After completion, reaction mass quenched by water (30 mL) and extracted with DCM (3×50 mL), organic layer was collected and dried over sodium sulphate and evaporate under reduced pressure to get crude and crude was purified by flash column chromatography (SiO₂: 0-10% ethyl acetate in hexane) to give 6-{[4-(tert-butoxycarbonylamino)butyl]{2-[(tert-butyl)bis(methyl)siloxy]-7-(1-octylnonylcarbonyloxy)heptyl}amino}-5-[(tert-butyl)bis(methyl)siloxy]hexyl undecanoate [14](6.2 g, 79.18% Yield) as reddish liquid.

### Result:

ELSD analysis: Purity 95.63%, Calculated C₆₃H₁₂₃N₂O₈Si₂=1096.92, Observed=1097.55 (m/z, M+H⁺).

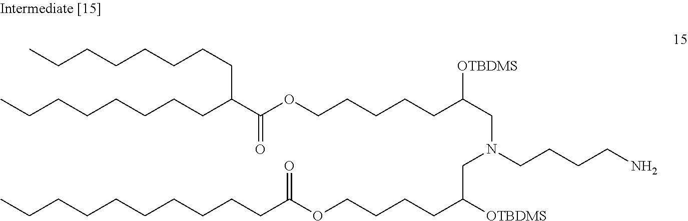 [SMILES: CCCCCCCCCCC(=O)OCCCCC(CN(CCCCN)CC(CCCCCOC(=O)C(CCCCCCCC)CCCCCCCC)O[Si](C)(C)C(C)(C)C)O[Si](C)(C)C(C)(C)C]

As depicted in Scheme 31: To stirred solution of 6-{[4-(tert-butoxycarbonylamino)butyl]{2-[(tert-butyl)bis(methyl) siloxy]-7-(1-octylnonylcarbonyloxy)heptyl}amino}-5-[(tert-butyl)bis(methyl)siloxy]hexyl undecanoate [14](1.2 g, 1.09 mmol) in dichloromethane (12 mL, 187 mmol) added trifluoroacetic acid (1.87 g, 16.4 mmol) dropwise at cooling condition and allow to stirred at RT for 3 hr. The progress of reaction was monitored by TLC. After completion, reaction mass quenched by saturated aqueous sodium bicarbonate solution and extract with DCM (3×15 mL), organic layer was collected dried over sodium sulphate, evaporate under reduced pressure to get 6-[(4-aminobutyl){2-[(tert-butyl)bis(methyl)siloxy]-7-(1-octylnonylcarbonyloxy)heptyl}amino]-5-[(tert-butyl)bis(methyl)siloxy]hexyl undecanoate [15](1.0 g 91.74% Yield) as pale yellow liquid. The crude used for next step without further purification.

### Result:

ELSD analysis: Purity 99.13%, Calculated C₅₈H₁₂₀N₂O₆Si₂=996.87, Observed=997.50 (m/z, M+H⁺).

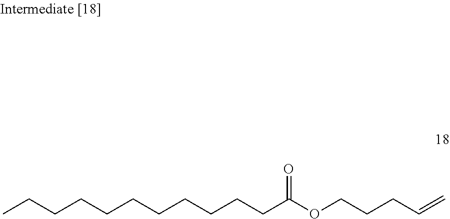 [SMILES: C=CCCCOC(=O)CCCCCCCCCCC]

As depicted in Scheme 31: To a stirred solution of lauric acid [16](25.6 g, 128 mmol) in dichloromethane (0.6 L, 9.37 mol), were added N,N-dimethyl-4-pyridylamine (7.09 g, 58 mmol) and EDC·HCl (26.7 g, 139 mmol) followed by 4-penten-1-ol [17](10 g, 116 mmol) at room temperature. Reaction mass was allowed to stirred for 16 h at room temperature. The progress of reaction was monitored by ELSD/TLC (SM was consumed). Water (300 ml) was added to the reaction mixture and extract with DCM (2×500 mL). The separated organic layer was collect, combined, dried over anhy. Na₂SO₄ and concentrated under reduce pressure to obtain crude product. The crude product of 4-pentenyl dodecanoate [18](23.0 g, 73.8% Yield) was forwarded to next step without further purification.

### Result:

¹H NMR (400 MHz, CDCl3): δ 5.83-5.75 (m, 1H), 5.06-4.97 (m, 2H), 4.09-4.06 (t, J=6.8 Hz, 2H), 2.32-2.25 (t, J=7.6 Hz, 2H), 2.15-2.09 (q, J=7.6 Hz, 2H), 1.75-1.68 (m, 2H), 1.62-1.57 (m, 2H), 1.35-1.23 (m, 16H), 0.89-0.86 (t, J=6.8 Hz, 3H) ppm.

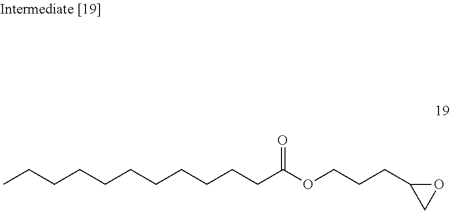 [SMILES: CCCCCCCCCCCC(=O)OCCCC1CO1]

As depicted in Scheme 31: To a stirred solution of 4-pentenyl dodecanoate [18](23.0 g, 85.7 mmol) in dichloromethane (300 mL) was added m-chlorobenzeneperoxycarboxylic acid (29.6 g, 171 mmol) at room temperature. The reaction mixture was stirred for 16 h at room temperature. The progress of reaction was monitored by ELSD/TLC (SM was consumed). After completion of reaction, reaction mixture was quenched with saturated sodium bicarbonate solution. Water (500 mL) was added to the reaction mixture and extracted with DCM (2×500 ml). The organic layer was separated, dried over Na₂SO₄ and concentrated under reduce pressure to ger crude. The crude was purified by flash column chromatography (SiO₂: 0-5% EtOAc/Heptane) to obtain 3-(2-oxiranyl)propyl dodecanoate [19] (10 g, 41.03% Yield) as colourless liquid.

### Result:

¹H NMR (400 MHz, CDCl₃): δ 4.13-4.09 (m, 2H), 2.95-2.92 (m, 1H), 2.78-2.75 (t, J=4.4 Hz, 1H), 2.49.75-2.47 (m, 1H), 2.30-2.27 (t, J=7.6 Hz, 2H), 1.82-1.75 (m, 2H), 1.67-1.56 (m, 4H), 1.32-1.20 (m, 16H), 0.89-0.86 (t, J=6.8 Hz, 3H) ppm.

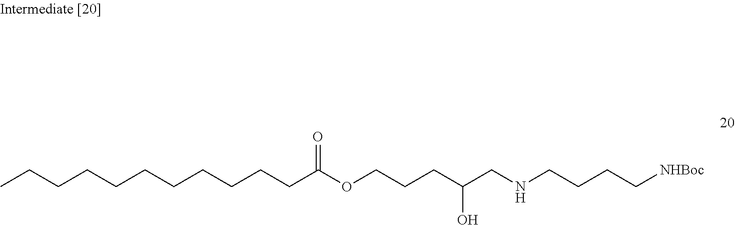 [SMILES: CCCCCCCCCCCC(=O)OCCCC(O)CNCCCCNC(=O)OC(C)(C)C]

As depicted in Scheme 31: To a stirred solution of tert-butyl (4-aminobutyl)carbamate [11](11.0 g, 58.4 mmol) in isopropanol (220 mL) was added 3-(2-oxiranyl)propyl dodecanoate [19](15 g, 52.6 mmol) slowly at RT. The reaction mixture was stirred at room temperature for 72 h. The progress of reaction was monitored by ELSD/TLC. After completion of reaction (starting material was consumed), the reaction mixture was concentrated under reduced pressure to get crude. The crude was purified by flash column chromatography (SiO₂: 0-10% MeOH/DCM) to obtain 5-[4-(tert-butoxycarbonylamino)butylamino]-4-hydroxypentyl dodecanoate [20](8.0 g, 29% Yield) as a colourless liquid.

### Result:

ELSD analysis: Purity 99.94%, Calculated C₂₆H₅₂N₂O₅=472.39, Observed=473.40 (m/z, M+H⁺).

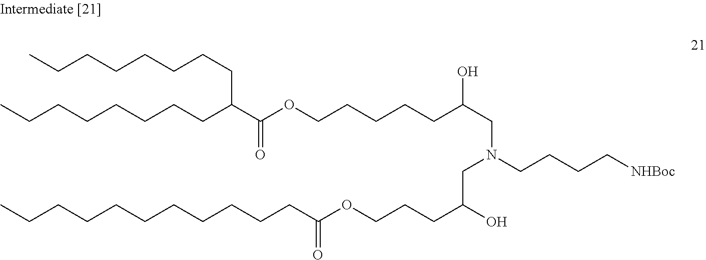 [SMILES: CCCCCCCCCCCC(=O)OCCCC(O)CN(CCCCNC(=O)OC(C)(C)C)CC(O)CCCCCOC(=O)C(CCCCCCCC)CCCCCCCC]

As depicted in Scheme 31: To a stirred solution of 5-[4-(tert-butoxycarbonylamino)butylamino]-4-hydroxypentyl dodecanoate [20](4.33 g, 9.16 mmol) in isopropanol (100 mL), was added N-ethylbis(isopropyl)amine (3.55 g, 27.5 mmol) followed by the addition of 5-(2-oxiranyl)pentyl 2-octyldecanoate [6](4 g, 10.1 mmol) at room temperature. The reaction mixture was stirred for 16 hr at 90° C. The progress of reaction was monitored by ELSD/TLC. After completion of reaction, the reaction mixture was concentrated under reduced pressure to get crude. The crude was purified by flash column chromatography (SiO₂: 0-10% MeOH/DCM) to get the desired compound 5-{[4-(tert-butoxycarbonylamino)butyl][2-hydroxy-7-(1-octylnonylcarbonyloxy) heptyl]amino}-4-hydroxypentyl dodecanoate [21](5.5 g, 69.09% Yield) as a pale yellow viscous liquid.

### Result:

ELSD analysis: Purity 98.72%, Calculated C₅₁H₁₀₀N₂O₈=868.75, Observed=869.45 (m/z, M+H⁺).

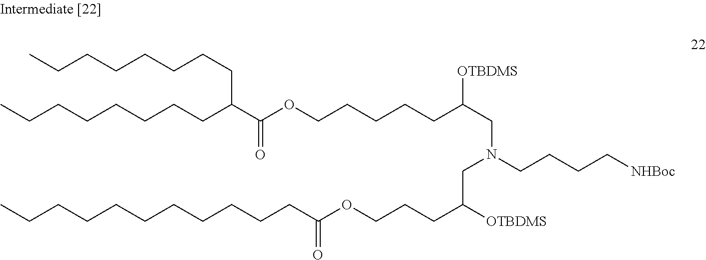 [SMILES: CCCCCCCCCCCC(=O)OCCCC(CN(CCCCNC(=O)OC(C)(C)C)CC(CCCCCOC(=O)C(CCCCCCCC)CCCCCCCC)O[Si](C)(C)C(C)(C)C)O[Si](C)(C)C(C)(C)C]

As depicted in Scheme 31: To a stirred solution of 5-{[4-(tert-butoxycarbonylamino)butyl][2-hydroxy-7-(1-octylnonylcarbonyloxy)heptyl]amino}-4-hydroxypentyl dodecanoate [21](5.5 g, 6.33 mmol) in DCM (150 mL) was added imidazole (4.31 g, 63.3 mmol) and (tert-butyl)(chloro)bis (methyl)silane (7.63 g, 50.6 mmol). The reaction mixture was stirred at RT for 16 hr. The progress of reaction was monitored by TLC and ELSD data. After completion of reaction, reaction mass quenched by water (50 ml) and extracted with DCM (3×50 mL). The organic layer was dried over sodium sulphate and concentrated under reduced pressure to get crude. The crude was purified by flash column chromatography (SiO₂: 0-10% ethyl acetate in hexane) to give 5-{[4-(tert-butoxycarbonylamino)butyl]{2-[(tert-butyl)bis(methyl)siloxy]-7-(1-octylnonylcarbonyloxy)heptyl}amino}-4-[(tert-butyl)bis(methyl)siloxy]pentyl dodecanoate [22](6 g, 86.38% Yield) as a reddish liquid.

ELSD analysis: Purity 95.64%, Calculated C₆₃H₁₂₃N₂O₈Si₂=1096.92, Observed=1097.55 (m/z, M+H⁺).

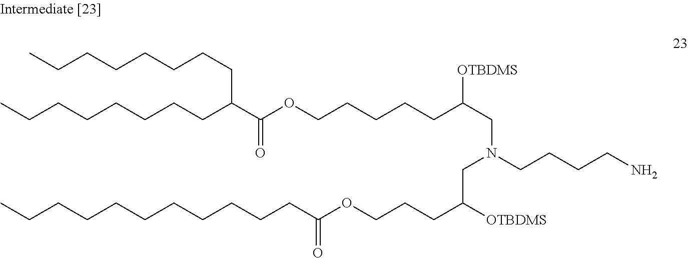 [SMILES: CCCCCCCCCCCC(=O)OCCCC(CN(CCCCN)CC(CCCCCOC(=O)C(CCCCCCCC)CCCCCCCC)O[Si](C)(C)C(C)(C)C)O[Si](C)(C)C(C)(C)C]

As depicted in Scheme 31: To a stirred solution of 5-{[4-(tert-butoxycarbonylamino)butyl]{2-[(tert-butyl)bis(methyl) siloxy]-7-(1-octylnonylcarbonyloxy)heptyl}amino}-4-[(tert-butyl)bis(methyl)siloxy]pentyl dodecanoate [22](1 g, 911 μmol) in dichloromethane (10 mL) was added trifluoroacetic acid (1.05 mL, 13.7 mmol) drop wise at cooling condition. The reaction mixture was stirred at RT for 3 hr. The progress of reaction was monitored by TLC. After completion of reaction, reaction was quenched with saturated sodium bicarbonate and extracted with DCM (3×15 mL). The organic layer was dried over sodium sulphate, evaporate under reduced pressure to get (0.9 g, Crude) as pale yellow liquid, which was used in next step without further purification.

### Result:

ELSD analysis: Purity 98.75%, Calculated C₅₈H₁₂₀N₂O₆Si₂=996.87, Observed=997.50 (m/z, M+H⁺).

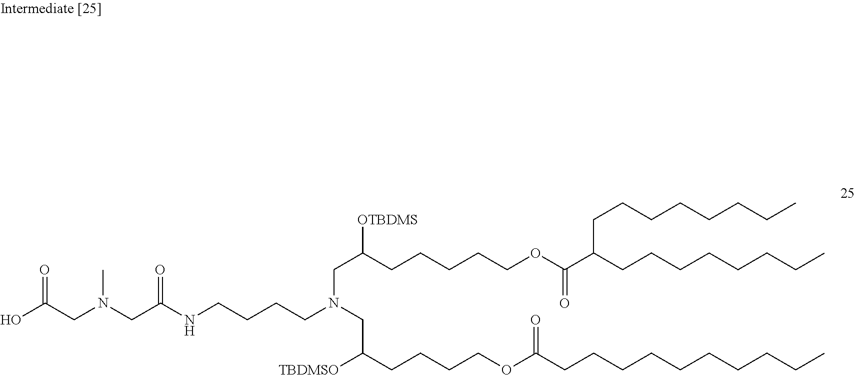 [SMILES: CCCCCCCCCCC(=O)OCCCCC(CN(CCCCNC(=O)CN(C)CC(=O)O)CC(CCCCCOC(=O)C(CCCCCCCC)CCCCCCCC)O[Si](C)(C)C(C)(C)C)O[Si](C)(C)C(C)(C)C]

As depicted in Scheme 31: To a stirred solution of [(carboxymethyl)-N-methylamino]acetic acid [24](0.3 g, 2.04 mmol) in tetrahydrofuran (15 mL) and dimethylformamide (3 mL) added 1H-1,2,3-benzotriazol-1-yloxytridimethylaminophosphonium hexafluoridophosphate(1-) (1.22 mL, 2.04 mmol) followed by N-ethylbis(isopropyl)amine (395 mg, 3.06 mmol) and allow to stirred reaction mix. to stirred at RT for 6 hr, reaction progress was monitored by TLC. After completion, 6-((4-aminobutyl)(2-((tert-butyldimethylsilyl)oxy)-7-((2-octyldecanoyl)oxy)heptyl)amino)-5-((tert-butyldimethylsilyl)oxy)hexyl undecanoate [15](906 mg, 908 μmol) was added into reaction mass at RT. The reaction mixture was stirred at RT for next 16 h. The progress of reaction was monitored by ELSD and TLC. After completion, reaction was quenched with water (15 mL) and extracted with DCM (3×25 mL). The organic layer was dried over sodium sulphate and concentrated under reduced pressure to get crude. The crude was purified by flash column chromatography (SiO₂: 0-10% methanol in DCM) to obtain 7-(2-((tert-butyldimethylsilyl)oxy)-6-(undecanoyloxy)hexyl)-2,2,3,3,15-pentamethyl-5-(5-((2-octyldecanoyl)oxy)pentyl)-13-oxo-4-oxa-7,12,15-triaza-3-silaheptadecan-17-oic acid [25](0.85 g, 37% Yield) as reddish liquid.

### Result:

ELSD analysis: Purity 99.03%, Calculated C₆₃H₁₂₇N₃O₉Si₂=1125.91, Observed=1126.40 (m/z, M+H⁺).

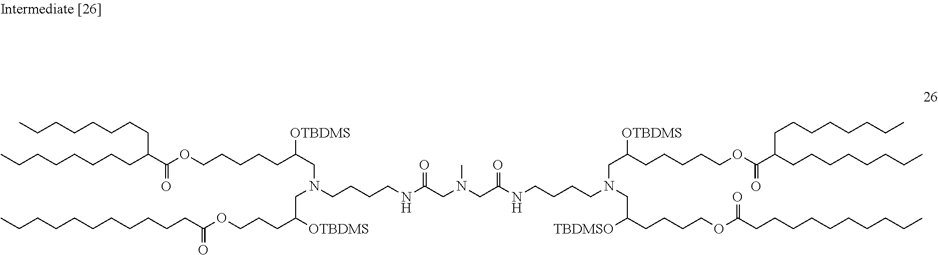 [SMILES: CCCCCCCCCCCC(=O)OCCCC(CN(CCCCNC(=O)CN(C)CC(=O)NCCCCN(CC(CCCCCOC(=O)C(CCCCCCCC)CCCCCCCC)O[Si](C)(C)C(C)(C)C)CC(CCCCOC(=O)CCCCCCCCCC)O[Si](C)(C)C(C)(C)C)CC(CCCCCOC(=O)C(CCCCCCCC)CCCCCCCC)O[Si](C)(C)C(C)(C)C)O[Si](C)(C)C(C)(C)C]

As depicted in Scheme 31: To a stirred solution of ({[N-4-({2-[(tert-butyl)bis(methyl)siloxy]-6-(decylcarbonyloxy) hexyl}{2-[(tert-butyl)bis(methyl)siloxy]-7-(1-octylnonylcarbonyloxy) heptyl}amino) butylcarbamoyl]methyl}-N-methylamino)acetic acid (850 mg, 754 μmol) in dichloromethane (10 mL) was added N,N-dimethyl-4-pyridylamine (46.1 mg, 377 μmol), and 2-methyl-2,6,8-triaza-6,7-decadiene-hydrogen chloride (1/1) (217 mg, 1.13 mmol) followed by 5-[(4-aminobutyl){2-[(tert-butyl)bis(methyl)siloxy]-7-(1-octylnonylcarbonyloxy) heptyl}amino]-4-[(tert-butyl)bis(methyl)siloxy]pentyl dodecanoate [23](677 mg, 679 μmol) at room temperature under inert atmosphere. The resulting reaction mass was stirred at RT for 48 h. The progress of reaction was monitored by TLC and ELSD mass analysis. After completion, reaction was quenched by water (5 ml) and extracted with DCM (2×50 mL). The organic layer was dried over anhy. sodium sulphate and concentrated under reduced pressure to get crude. The crude was purified by flash column chromatography (SiO2: 0-10% EA/Heptane) to obtain 6,26-bis((tert-butyldimethylsilyl)oxy)-8-(2-((tert-butyldimethylsilyl) oxy)-5-(dodecanoyloxy)pentyl)-24-(2-((tert-butyldimethylsilyl)oxy)-6-(undecanoyloxy) hexyl)-16-methyl-14,18-dioxo-8,13,16,19,24-pentaazahentriacontane-1,31-diyl bis(2-octyldecanoate) [26] (0.5 g, 31.47% Yield) as a colourless liquid.

### Result:

ELSD analysis: Purity 99.45%, Calculated C₁₂₁H₂₄₅N₅O₁₄Si₄=2104.77, Observed=1053.65 (m/z, M/2+H⁺).

### Synthesis of Compound LXXVIII

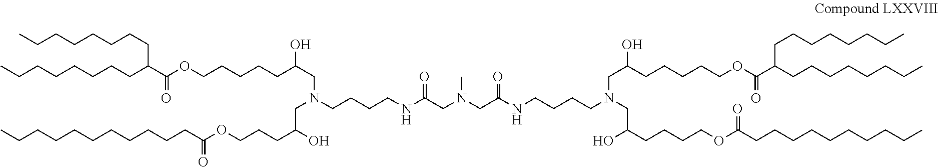 [SMILES: CCCCCCCCCCCC(=O)OCCCC(O)CN(CCCCNC(=O)CN(C)CC(=O)NCCCCN(CC(O)CCCCCOC(=O)C(CCCCCCCC)CCCCCCCC)CC(O)CCCCOC(=O)CCCCCCCCCC)CC(O)CCCCCOC(=O)C(CCCCCCCC)CCCCCCCC]

As depicted in Scheme 31: To a stirred solution of 6,26-bis((tert-butyldimethylsilyl)oxy)-8-(2-((tert-butyldimethylsilyl)oxy)-5-(dodecanoyloxy)pentyl)-24-(2-((tert-butyldimethylsilyl)oxy)-6-(undecanoyloxy)hexyl)-16-methyl-14,18-dioxo-8,13,16,19,24-pentaazahentriacontane-1,31-diyl bis(2-octyldecanoate) [26](0.5 g, 237 μmol) in tetrahydrofuran (5 mL) was added hydrogen fluoride pyridine (823 mg, 8.31 mmol) drop wise at cooling condition under nitrogen atmosphere. The reaction mixture was stirred at RT for 16 h. The progress of reaction was monitored by ELSD and TLC. After completion, reaction mass was diluted with diethyl ether (10 ml) and ice cold water (5 ml). To the above solution was added saturated sodium bicarbonate (pH 7) and the organic layer was separated. The organic layer was washed with Milli Q water (2×8 mL), dried over sodium sulphate and concentrated under reduced pressure to get crude compound. The crude was purified by flash column chromatography (SiO₂: 0-10% methanol in DCM) to get 8-(5-(dodecanoyloxy)-2-hydroxypentyl)-6,26-dihydroxy-24-(2-hydroxy-6-(undecanoyloxy)hexyl)-16-methyl-14,18-dioxo-8,13,16,19,24-pentaazahentriacontane-1,31-diylbis(2-octyldecanoate) [Compound LXXVIII] (0.1 g, 25.54% Yield) as a pale yellow liquid.

### Result:

¹H NMR (400 MHz, CDCl₃): δ 7.3-7.10 (brs, 2H), 4.14-4.00 (m, 8H), 3.74-3.58 (m, 4H), 3.40-3.22 (m, 4H), 3.13-3.07 (m, 4H), 2.63-2.24 (m, 26H), 1.86-1.20 (m, 123H), 0.89-0.86 (t, J=6.8 Hz, 18H) ppm.

ELSD analysis: Purity 99.45%, Calculated C₉₇H₁₈₉N₅O₁₄=1648.42, Observed=1650.55 (m/z, M+H⁺).

### Example 32: Synthesis of Compound LXXIX

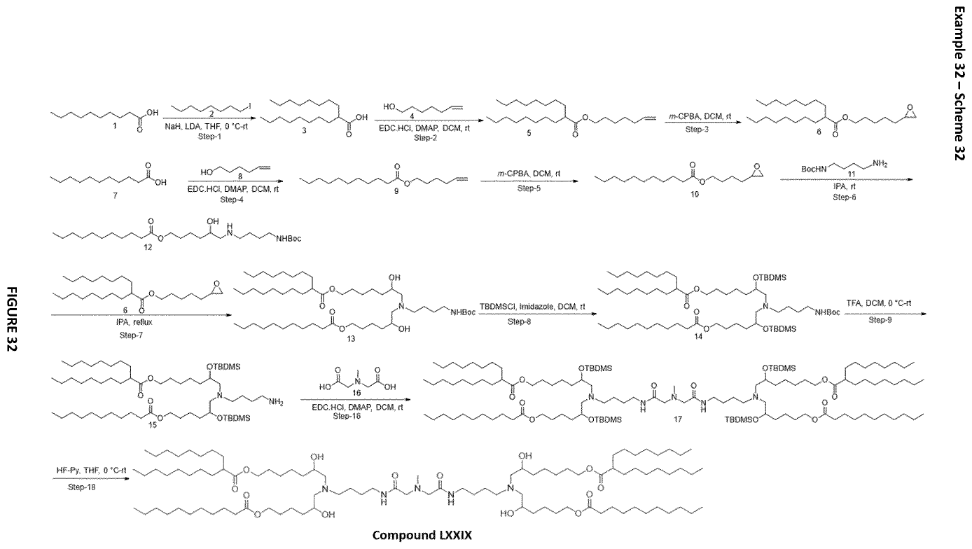

For example, the compounds of the invention may be prepared according to Scheme 32 (as depicted in FIG. 32).

### Intermediate [15]

Synthesis procedure of Intermediate [15] is given in the synthesis for Compound LXXVIII.

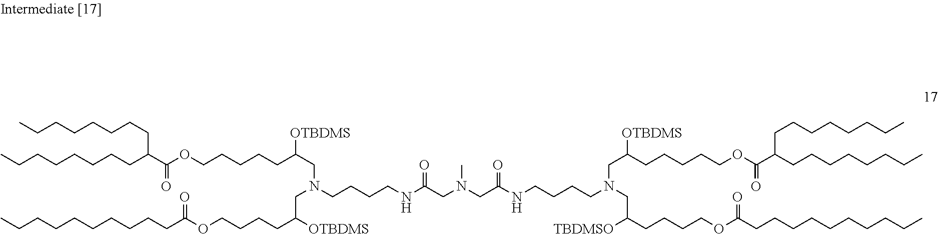 [SMILES: CCCCCCCCCCC(=O)OCCCCC(CN(CCCCNC(=O)CN(C)CC(=O)NCCCCN(CC(CCCCCOC(=O)C(CCCCCCCC)CCCCCCCC)O[Si](C)(C)C(C)(C)C)CC(CCCCOC(=O)CCCCCCCCCC)O[Si](C)(C)C(C)(C)C)CC(CCCCCOC(=O)C(CCCCCCCC)CCCCCCCC)O[Si](C)(C)C(C)(C)C)O[Si](C)(C)C(C)(C)C]

As depicted in Scheme 32: To a stirred solution of [(carboxymethyl)-N-methylamino]acetic acid [16](0.5 g, 3.4 mmol) in dichloromethane (50 mL) were added N,N-dimethyl-4-pyridylamine (208 mg, 1.7 mmol) and EDC·HCl (977 mg, 5.1 mmol) at RT. The reaction mixture was stirred at RT for 15 min and then added 6-[(4-aminobutyl){2-[(tert-butyl)bis(methyl)siloxy]-7-(1-octylnonyl carbonyloxy)heptyl}amino]-5-[(tert-butyl)bis (methyl)siloxy]hexyl undecanoate [15](3.05 g, 3.06 mmol). The reaction mixture was stirred RT for 16 h. The progress of reaction was monitored by ELSD data and TLC. After completion the reaction, reaction mass quenched with water (15 mL) and extracted with DCM (3×25 ml). The organic layer was collected, dried over sodium sulphate and concentrated under reduced pressure to get crude. The crude was purified by flash column chromatography (SiO₂: 0-10% methanol in DCM to obtain 5,25-bis((tert-butyldimethylsilyl)oxy)-7,23-bis(2-((tert-butyldimethylsilyl)oxy)-7-((2-octyldecanoyl)oxy) heptyl)-15-methyl-13,17-dioxo-7,12,15,18,23-pentaazanonacosane-1,29-diyldiundecanoate [17] (1.4 g 19.55% Yield) as a pale yellow liquid.

### Result:

ELSD analysis: Purity 95.39%, Calculated C₁₂₁H₂₄₅N₅O₁₄Si₄=2104.77, Observed=1053.65 (m/z, M/2+H⁺).

### Synthesis of Compound LXXIX

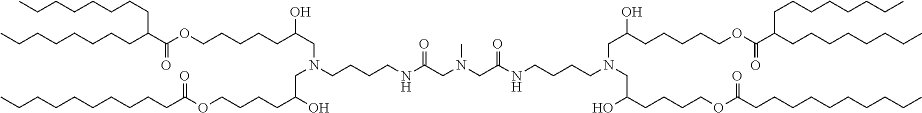 [SMILES: CCCCCCCCCCC(=O)OCCCCC(O)CN(CCCCNC(=O)CN(C)CC(=O)NCCCCN(CC(O)CCCCCOC(=O)C(CCCCCCCC)CCCCCCCC)CC(O)CCCCOC(=O)CCCCCCCCCC)CC(O)CCCCCOC(=O)C(CCCCCCCC)CCCCCCCC]

As depicted in Scheme 32: To stirred solution of 5,25-bis((tert-butyldimethylsilyl)oxy)-7,23-bis(2-((tert-butyldimethylsilyl)oxy)-7-((2-octyldecanoyl)oxy)heptyl)-15-methyl-13,17-dioxo-7,12,15,18,23-pentaazanonacosane-1,29-diyl diundecanoate5,25-bis((tert-butyldimethylsilyl) oxy)-7,23-bis(2-((tert-butyldimethylsilyl)oxy)-7-((2-octyldecanoyl)oxy)heptyl)-15-methyl-13,17-dioxo-7,12,15,18,23-pentaazanonacosane-1,29-diyl diundecanoate [14](1.4 g, 665 μmol) in tetrahydrofuran (14.0 mL) was added hydrogen fluoride pyridine (2.31 g, 23.3 mmol) dropwise at cooling condition under nitrogen atmosphere. The reaction mixture was stirred at RT for 16 h. The progress of reaction was monitored by ELSD data and TLC. After completion of the reaction, reaction mass was diluted with diethyl ether (50 mL) and ice cold water (20 mL). The organic layer was separated and washed with saturated sodium bicarbonate (20 mL), Milli Q water (2×15 mL). The organic layer was collected, dried over sodium sulphate and concentrated under reduced pressure to obtain 5,25-dihydroxy-7,23-bis(2-hydroxy-7-((2-octyldecanoyl)oxy)heptyl)-15-methyl-13,17-dioxo-7,12,15,18,23-pentaazanonacosane-1,29-diyldiundecanoate [Compound LXXIX] (0.5 g, 45.61% Yield) as a pale yellow liquid.

### Results:

¹H-NMR (400 MHz, CDCl3): δ 7.21-6.81 (brm, 2H), 4.05 (t, J=6.0 Hz, 8H), 3.75-3.65 (brs, 4H), 3.42-3.20 (brm, 4H), 3.12-3.07 (brm, 4H), 2.65-2.58 (m, 10H), 2.38 (s, 3H), 2.30-2.62 (m, 8H), 1.66-1.51 (m, 28H), 1.48-1.33 (m, 20H), 1.32-1.18 (brm, 80H), 0.87 (t, J=6.8 Hz, 18H).

ELSD analysis: Purity 99.24%, Calculated C₉₇H₁₈₉N₅O₁₄=1648.42, Observed=1649.60 (m/z, M+H⁺).

### Example 33: Synthesis of Compound LXXX

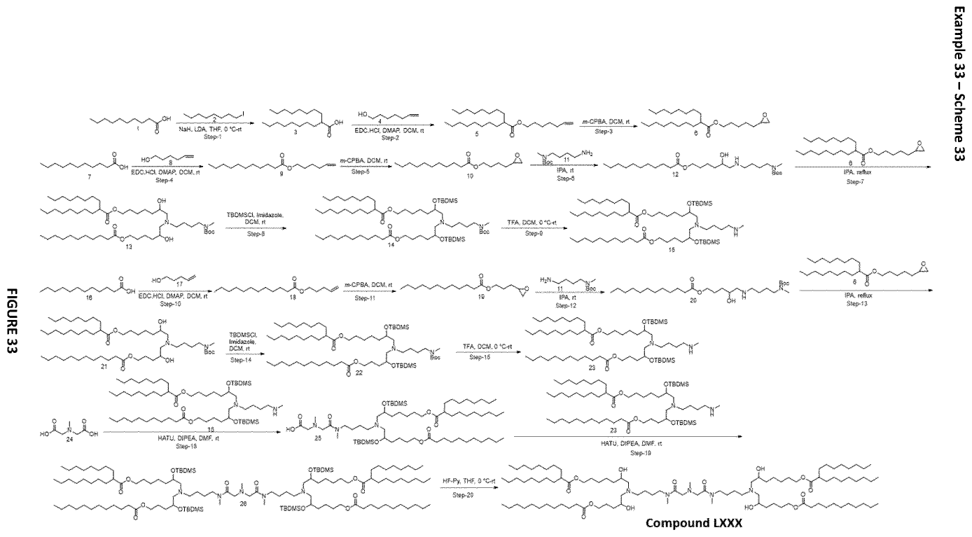

For example, the compounds of the invention may be prepared according to Scheme 33 (as depicted in FIG. 33).

### Intermediate [6], Intermediate [10] and Intermediate [19]

As depicted in Scheme 33: Synthesis procedure of Intermediate [6], Intermediate [10] and Intermediate [19] is given in the synthesis for Compound LXXVIII.

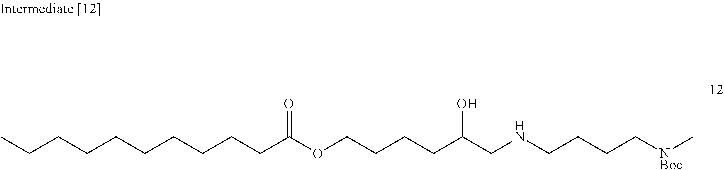 [SMILES: CCCCCCCCCCC(=O)OCCCCC(O)CNCCCCN(C)C(=O)OC(C)(C)C]

As depicted in Scheme 33: To a stirred solution of tert-butyl (4-aminobutyl)(methyl)carbamate [11](3 g, 14.8 mmol) in isopropanol (150 mL) was added 4-(2-oxiranyl)butyl undecanoate [10](4.22 g, 14.8 mmol) and stirred at RT for 3 days. The progress of reaction was monitored by TLC (SM was consumed). After completion of the reaction, reaction mass was concentrated under reduce pressure to get crude. The crude was purified by flash column chromatography (SiO₂: 0-10% Methanol in DCM) to obtain 6-[4-(N-tert-butoxycarbonyl-N-methylamino)butylamino]-5-hydroxyhexyl undecanoate [12](2.6 g, 36.02% Yield) as colourless liquid.

### Result:

ELSD analysis: Purity 99.67%, Calculated C₂₇H₅₄N₂O₅=486.40, Observed=487.30 (m/z, M+H⁺).

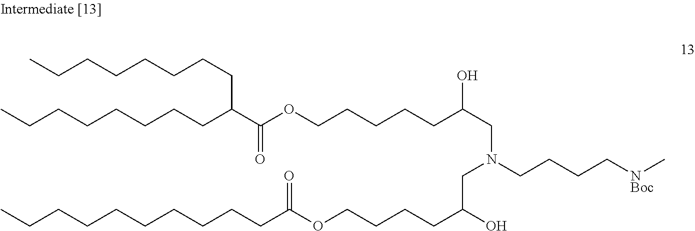 [SMILES: CCCCCCCCCCC(=O)OCCCCC(O)CN(CCCCN(C)C(=O)OC(C)(C)C)CC(O)CCCCCOC(=O)C(CCCCCCCC)CCCCCCCC]

As depicted in Scheme 33: To a stirred solution of 6-({4-[(tert-butyl)(oxycarbonylamino)]butyl}-N-methylamino)-5-hydroxyhexyl undecanoate [12](2.78 g, 5.71 mmol) in isopropanol (49.1 mL) was added N-ethylbis(isopropyl)amine (2.98 mL, 17.1 mmol) followed by the addition of 5-(2-oxiranyl)pentyl 2-octyldecanoate [6](2.49 g, 6.28 mmol) at room temperature. The reaction mixture was stirred at 0° C. for 64 h. The progress of reaction was monitored by TLC/ELSD. After completion of the reaction, reaction mass was concentrated under reduced pressure to get crude. The crude was purified by flash column chromatography (SiO₂: 0-10% MeOH in DCM) to obtain 6-({4-[(tert-butyl)(methyl)(oxycarbonylamino)]butyl}[2-hydroxy-7-(1-octylnonylcarbonyloxy)heptyl]amino)-5-hydroxyhexyl undecanoate [13](3.3 g, 69.93% Yield) as a pale yellow liquid.

### Result:

ELSD analysis: Purity 99.26%, Calculated C₅₂H₁₀₂N₂O₈=882.76, Observed=883.40 (m/z, M+H⁺).

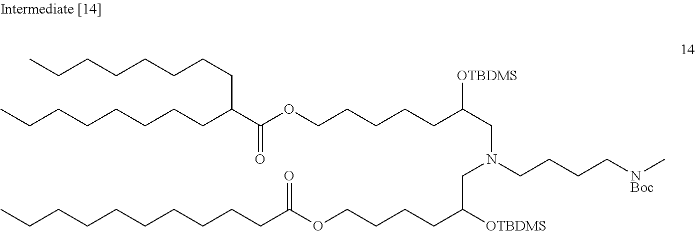 [SMILES: CCCCCCCCCCC(=O)OCCCCC(CN(CCCCN(C)C(=O)OC(C)(C)C)CC(CCCCCOC(=O)C(CCCCCCCC)CCCCCCCC)O[Si](C)(C)C(C)(C)C)O[Si](C)(C)C(C)(C)C]

As depicted in Scheme 33: To a stirred solution of 6-({4-[(tert-butyl)(methyl)(oxycarbonylamino)]butyl}[2-hydroxy-7-(1-octylnonylcarbonyloxy)heptyl]amino)-5-hydroxyhexyl undecanoate [13](3.3 g, 3.74 mmol) in dichloromethane (105 mL) were added imidazole (2.54 g, 37.4 mmol) and (tert-butyl)(chloro)bis(methyl)silane (4.5 g, 29.9 mmol). The reaction mixture was stirred at RT for 16 h. The progress of reaction was monitored by TLC and ELSD data. After completion of the reaction, reaction mass quenched by water (30 mL) and extracted with DCM (3×50 mL). The organic layer was collected and dried over sodium sulphate and concentrated under reduced pressure to get crude. The crude was purified by flash column chromatography (SiO₂: 0-10% ethyl acetate in hexane to give as 6-((4-((tert-butoxycarbonyl)(methyl)amino)butyl)(2-((tert-butyldimethylsilyl)oxy)-7-((2-octyldecanoyl)oxy)heptyl)amino)-5-((tert-butyldimethylsilyl) oxy)hexyl undecanoate [14](3.2 g, 77.04% Yield) as a pale yellow liquid.

### Result:

ELSD analysis: Purity 98.82%, Calculated C₆₄H₁₃₀N₂O₃Si₂=1110.94, Observed=1111.40 (m/z, M+H⁺).

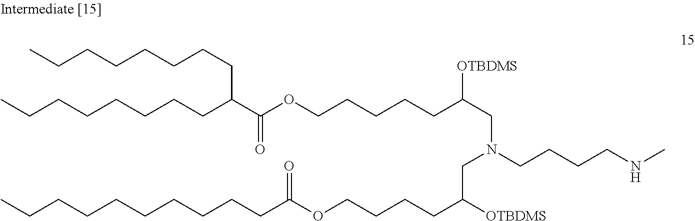 [SMILES: CCCCCCCCCCC(=O)OCCCCC(CN(CCCCNC)CC(CCCCCOC(=O)C(CCCCCCCC)CCCCCCCC)O[Si](C)(C)C(C)(C)C)O[Si](C)(C)C(C)(C)C]

As depicted in Scheme 33: To a stirred solution of 6-((4-((tert-butoxycarbonyl)(methyl)amino)butyl)(2-((tert-butyldimethylsilyl)oxy)-7-((2-octyldecanoyl)oxy)heptyl)amino)-5-((tert-butyldimethylsilyl) oxy)hexyl undecanoate [14](3 g, 2.7 mmol) in dichloromethane (25 mL) was added trifluoroacetic acid (3.1 mL, 40.5 mmol) at cooling condition. The reaction mixture was stirred at RT for 4 h. The progress of reaction was monitored by TLC. After completion of the reaction, reaction mass was quenched by saturated sodium bicarbonate (upto pH=8) and extracted with DCM (3×20 mL). The organic layer was collected, dried over sodium sulphate and concentrated under reduced pressure to get 5-((tert-butyldimethylsilyl)oxy)-6-((2-((tert-butyldimethylsilyl)oxy)-7-((2-octyldecanoyl)oxy)heptyl)(4-(methylamino)butyl)amino)hexyl undecanoate [15](2.76 g 98% Yield) as a pale yellow liquid.

### Result:

ELSD analysis: Purity 98.28%, Calculated C₅₉H₁₂₂N₂O₆Si₂=1010.88, Observed=1011.35 (m/z, M+H⁺).

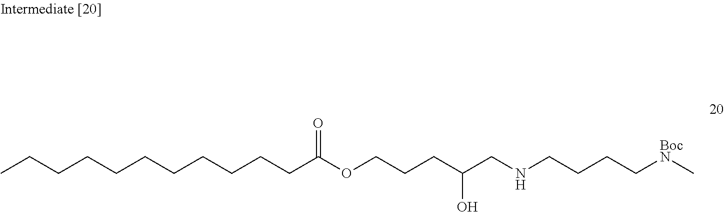 [SMILES: CCCCCCCCCCCC(=O)OCCCC(O)CNCCCCN(C)C(=O)OC(C)(C)C]

As depicted in Scheme 33: To a stirred solution of tert-butyl (4-aminobutyl)(methyl)carbamate [11](2 g, 9.89 mmol) in isopropanol (0.1 L, 1.31 mol) was added 3-(2-oxiranyl)propyl dodecanoate [19](2.81 g, 9.89 mmol) at RT. The reaction mixture was stirred at RT for 3 days. The progress of reaction was monitored by TLC (SM was consumed). After completion of the reaction, reaction mass was concentrated under reduced pressure to get crude. The crude was purified by flash column chromatography (SiO₂: 0-10% Methanol in DCM), to obtain 5-[4-(N-tert-butoxycarbonyl-N-methylamino)butylamino]-4-hydroxypentyl dodecanoate [20](1.8 g, 37.40% Yield) as a colourless liquid.

### Result:

ELSD analysis: Purity 99.89%, Calculated C₂₇H₅₄N₂O₅=486.40, Observed=487.35 (m/z, M+H⁺).

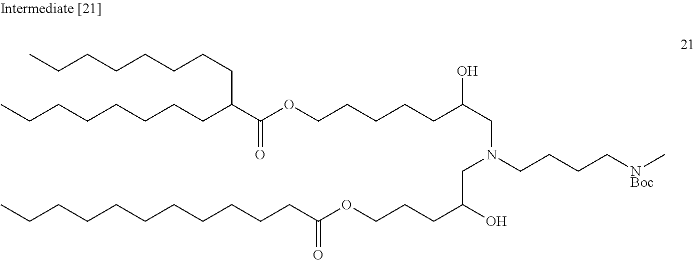 [SMILES: CCCCCCCCCCCC(=O)OCCCC(O)CN(CCCCN(C)C(=O)OC(C)(C)C)CC(O)CCCCCOC(=O)C(CCCCCCCC)CCCCCCCC]

As depicted in Scheme 33: To a stirred solution of 5-{4-[(tert-butyl)(methyl)(oxycarbonylamino)]butylamino}-4-hydroxypentyl dodecanoate [20](1.8 g, 3.7 mmol) in isopropanol (18.4 mL), were added N-ethylbis(isopropyl)amine (1.93 mL, 11.1 mmol) followed by 5-(2-oxiranyl)pentyl 2-octyldecanoate [6](1.61 g, 4.07 mmol) at room temperature. The reaction mixture was stirred for 64 hr at 90° C. The progress of reaction was monitored by ELSD/TLC. After completion of the reaction, IPA was evaporated under reduced pressure to get crude. The crude was purified by flash column chromatography (SiO₂: 0-10% MeOH/DCM) to get 5-((4-((tert-butoxycarbonyl)(methyl)amino)butyl)(2-hydroxy-7-((2-octyldecanoyl)oxy)heptyl)amino)-4-hydroxypentyl dodecanoate [21](2.3 g, 70.40% Yield) as a pale yellow viscous liquid.

### Result:

ELSD analysis: Purity 85.41%, Calculated C₅₂H₁₀₂N₂O₈=882.76, Observed=883.45 (m/z, M+H⁺)

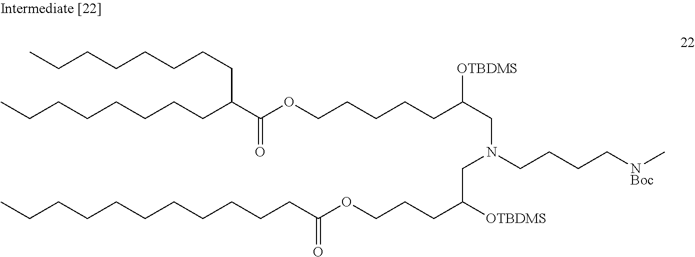 [SMILES: CCCCCCCCCCCC(=O)OCCCC(CN(CCCCN(C)C(=O)OC(C)(C)C)CC(CCCCCOC(=O)C(CCCCCCCC)CCCCCCCC)O[Si](C)(C)C(C)(C)C)O[Si](C)(C)C(C)(C)C]

As depicted in Scheme 33: To a stirred solution of 5-({4-[(tert-butyl)(methyl)(oxycarbonylamino)]butyl}[2-hydroxy-7-(1-octylnonylcarbonyloxy)heptyl]amino)-4-hydroxypentyl dodecanoate [21](2.3 g, 2.6 mmol) in DCM (60 mL) were added imidazole (1.77 g, 26 mmol) and (tert-butyl)(chloro)bis(methyl)silane (3.14 g, 20.8 mmol). The reaction mixture was stirred at RT for 16 h. The progress of reaction was monitored by TLC and ELSD data. After completion-of the reaction, reaction mass quenched with water (50 mL) and extracted with DCM (3×50 mL). The organic layer was collected and dried over sodium sulphate and concentrated under reduced pressure to get crude. The crude was purified by flash column chromatography (SiO₂: 0-10% ethyl acetate in hexane) to give 4-[(tert-butyl)bis(methyl)siloxy]-5-({2-[(tert-butyl)bis (methyl)siloxy]-7-(1-octylnonylcarbonyloxy)heptyl}{4-[(tert-butyl)(methyl) (oxycarbonylamino)]butyl}amino)pentyl dodecanoate [22](1.8 g, 62.17% Yield) as a pale yellow liquid.

### Result:

ELSD analysis: Purity 99.65%, Calculated C₆₄H₁₃₀N₂O₃Si₂=1110.94, Observed=1111.35 (m/z, M+H⁺).

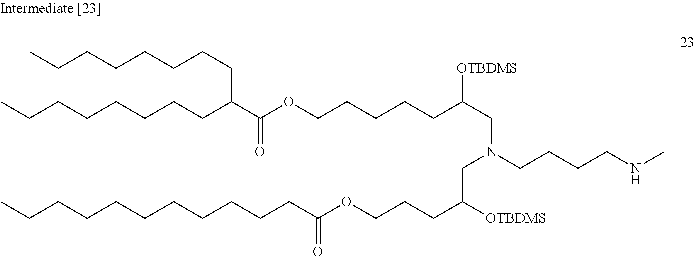 [SMILES: CCCCCCCCCCCC(=O)OCCCC(CN(CCCCNC)CC(CCCCCOC(=O)C(CCCCCCCC)CCCCCCCC)O[Si](C)(C)C(C)(C)C)O[Si](C)(C)C(C)(C)C]

As depicted in Scheme 33: To a stirred solution of 4-[(tert-butyl)bis(methyl)siloxy]-5-({2-[(tert-butyl)bis(methyl)siloxy]-7-(1-octylnonylcarbonyloxy)heptyl}{4-[(tert-butyl)(methyl)(oxycarbonylamino)]butyl}amino)pentyl dodecanoate [22](1.8 g, 1.62 mmol) in dichloromethane (20 mL) was added trifluoroacetic acid (1.86 mL, 24.3 mmol) dropwise at 0° C. The reaction mass was stirred at room temperature for 3 h. The progress of reaction was monitored by TLC. After completion of reaction, reaction mass was quenched with saturated solution of sodium bicarbonate (make pH 8-9) and extracted with DCM (2×5 mL). The combined organic layers was dried over anhydrous sodium sulphate and concentrated under reduced pressure to get crude of 4-[(tert-butyl)bis(methyl)siloxy]-5-({2-[(tert-butyl)bis(methyl)siloxy]-7-(1-octylnonylcarbonyloxy) heptyl}[4-(methylamino)butyl]amino)pentyl dodecanoate [23](1.6 g, 97.68% Yield) as a pale yellow liquid.

### Result:

ELSD analysis: Purity 99.53%, Calculated C₅₉H₁₂₂N₂O₆Si₂=1010.88, Observed=1011.35 (m/z, M+H⁺).

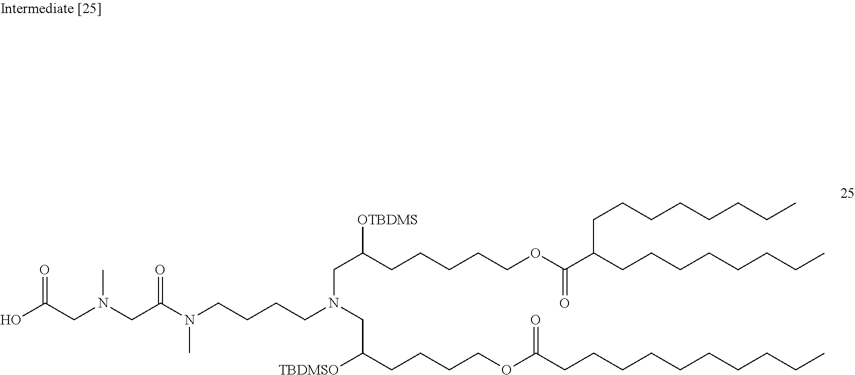 [SMILES: CCCCCCCCCCC(=O)OCCCCC(CN(CCCCN(C)C(=O)CN(C)CC(=O)O)CC(CCCCCOC(=O)C(CCCCCCCC)CCCCCCCC)O[Si](C)(C)C(C)(C)C)O[Si](C)(C)C(C)(C)C]

As depicted in Scheme 33: To a stirred solution of [(carboxymethyl)-N-methylamino]acetic acid [24](0.5 g, 3.4 mmol) in tetrahydrofuran (25 mL) and dimethylformamide (5 mL) was added 1H-1,2,3-benzotriazol-1-yloxytridimethylaminophosphonium hexafluoridophosphate(1-) (1.5 g, 3.4 mmol) followed by the addition of N-ethylbis(isopropyl)amine (888 μL, 5.1 mmol). The reaction mixture was stirred at RT for 6 hr. The progress of reaction was monitored by TLC and ELSD. After that 4-[(tert-butyl)bis(methyl)siloxy]-5-({2-[(tert-butyl)bis(methyl)siloxy]-7-(1-octylnonyl carbonyloxy)heptyl}[4-(methylamino)butyl]amino)pentyl undecanoate [15](2.72 g, 2.72 mmol) was added. The reaction mixture was stirred at RT for 16 h. The progress of reaction was monitored by ELSD data and TLC. After completion of reaction, reaction mass quenched by water (15 mL) and extracted with DCM (3×25 mL). The organic layers were collected, combined, dried over anhy. sodium sulphate and concentrated under reduced pressure to get crude. The crude was purified by flash column chromatography (SiO₂: 0-10% methanol in DCM) to get [({[4-({2-[(tert-butyl)bis(methyl)siloxy]-6-(decylcarbonyloxy)hexyl}{2-[(tert-butyl)bis(methyl)siloxy]-7-(1-octylnonylcarbonyloxy)heptyl}amino)butyl]-N-methylcarbamoyl}methyl)-N-methylamino]acetic acid [25](1.5 g, 38.75% Yield) as a reddish liquid.

### Result:

ELSD analysis: Purity 96.48%, Calculated C₆₄H₁₂₉N₃O₉Si₂=1139.93, Observed=1140.40 (m/z, M+H⁺).

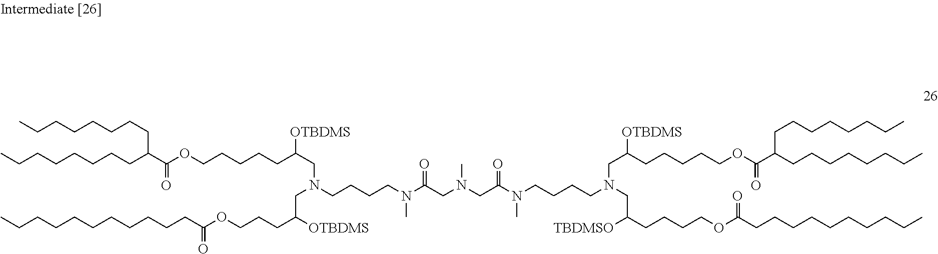 [SMILES: CCCCCCCCCCCC(=O)OCCCC(CN(CCCCN(C)C(=O)CN(C)CC(=O)N(C)CCCCN(CC(CCCCCOC(=O)C(CCCCCCCC)CCCCCCCC)O[Si](C)(C)C(C)(C)C)CC(CCCCOC(=O)CCCCCCCCCC)O[Si](C)(C)C(C)(C)C)CC(CCCCCOC(=O)C(CCCCCCCC)CCCCCCCC)O[Si](C)(C)C(C)(C)C)O[Si](C)(C)C(C)(C)C]

As depicted in Scheme 33: To a stirred solution of [({[4-({2-[(tert-butyl)bis(methyl)siloxy]-6-(decylcarbonyloxy) hexyl}{2-[(tert-butyl)bis(methyl)siloxy]-7-(1-octylnonylcarbonyloxy)heptyl}amino)butyl]-N-methylcarbamoyl}methyl)-N-methylamino]acetic acid [25](1.5 g, 1.3 mmol) in dimethylformamide (12 mL) were added to N-ethylbis(isopropyl)amine (421 mg, 3.25 mmol), and 1,1,3,3-tetramethyl-2-(3H-1,2,3,4-tetraazainden-3-yl)-3-isoureaium hexafluorido phosphate(1-) (742 mg, 1.95 mmol) followed by 4-[(tert-butyl)bis(methyl)siloxy]-5-({2-[(tert-butyl)bis(methyl)siloxy]-7-(1-octylnonylcarbonyloxy)heptyl}[4-(methylamino)butyl]amino)pentyl dodecanoate [23](1.32 g, 1.3 mmol). The reaction mixture was stirred at 60° C. for 16 h. The progress of reaction was monitored by ELSD data and TLC. After completion-of reaction, reaction mass quenched by brine solution (20 mL) and extracted with ethyl acetate (3×20 mL). The organic layers were collected, combined, dried over sodium sulphate and evaporate under reduced pressure to get crude. The crude was purified by flash column chromatography (SiO₂: 0-30% ethyl acetate in hexane) to get 6,26-bis((tert-butyldimethylsilyl)oxy)-8-(2-((tert-butyldimethylsilyl)oxy)-5-(dodecanoyloxy)pentyl)-24-(2-((tert-butyldimethylsilyl)oxy)-6-(undecanoyloxy)hexyl)-13,16,19-trimethyl-14,18-dioxo-8,13,16,19,24-pentaazahentriacontane-1,31-diyl bis(2-octyldecanoate) [26](1.2 g, 42.75% Yield) as a reddish liquid.

### Result:

ELSD analysis: Purity 98.16%, Calculated C₁₂₃H₂₄₉N₅O₁₄Si₄=2132.80, Observed=1067.60 (m/z, M/2+H⁺).

### Synthesis of Compound LXXX

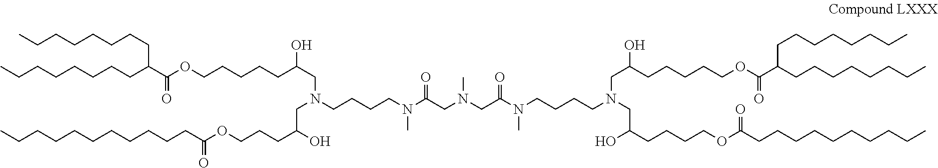 [SMILES: CCCCCCCCCCCC(=O)OCCCC(O)CN(CCCCN(C)C(=O)CN(C)CC(=O)N(C)CCCCN(CC(O)CCCCCOC(=O)C(CCCCCCCC)CCCCCCCC)CC(O)CCCCOC(=O)CCCCCCCCCC)CC(O)CCCCCOC(=O)C(CCCCCCCC)CCCCCCCC]

As depicted in Scheme 33: To a stirred solution of 5-{[4-(N-methyl{[({[4-({2-[(tert-butyl)bis(methyl)siloxy]-6-(decylcarbonyloxy)hexyl}{2-[(tert-butyl)bis(methyl)siloxy]-7-(1-octylnonylcarbonyloxy) heptyl}amino)butyl]-N-methylcarbamoyl}methyl)-N-methylamino]methyl}carbonylamino) butyl]{2-[(tert-butyl)bis(methyl)siloxy]-7-(1-octylnonylcarbonyloxy)heptyl}amino}-4-[(tert-butyl)bis(methyl)siloxy]pentyl dodecanoate [26](1.2 g, 562 μmol) in tetrahydrofuran (12 mL) was added hydrogen fluoride pyridine (2.53 mL, 28.1 mmol) drop wise at cooling condition under nitrogen atmosphere. The reaction mixture was stirred at RT for 16 h. The progress of reaction was monitored by ELSD data and TLC. After completion of reaction, reaction mass was diluted with diethyl ether (30 mL) and ice cold water (10 mL). The organic layer was separated and washed with saturated sodium bicarbonate (10 mL), Milli Q water (2×15 mL). The organic layer was collected, dried over sodium sulphate and convcentrated under reduced pressure to obtain 5-[(4-{N-methyl[({[(4-{[6-(decylcarbonyloxy)-2-hydroxyhexyl][2-hydroxy-7-(1-octylnonylcarbonyloxy)heptyl]amino}butyl)-N-methylcarbamoyl]methyl}-N-methylamino)methyl]carbonylamino}butyl)[2-hydroxy-7-(1-octylnonylcarbonyloxy) heptyl]amino]-4-hydroxypentyl dodecanoate [Compound LXXX](550 mg, 58.32% Yield) as pale yellow liquid.

### Results:

¹H-NMR (400 MHz, CDCl₃): δ 4.07 (m, 8H), 3.70-3.64 (m, 5H), 3.40-3.36 (m, 6H), 3.00-2.89 (m, 7H), 2.69-2.37 (m, 13H), 2.35-2.26 (m, 7H), 1.89-1.79 (brs, 1H), 1.67-1.53 (brm, 21H), 1.44-1.37 (brm, 20H), 1.31-1.19 (brm, 83H), 0.87 (t, J=6.8 Hz, 18H).

ELSD analysis: Purity 99.29%, Calculated C₉₉H₁₉₃N₅O₁₄=1676.45, Observed=1677.65 (m/z, M+H⁺).

### Example 34: Synthesis of Compound XCIII

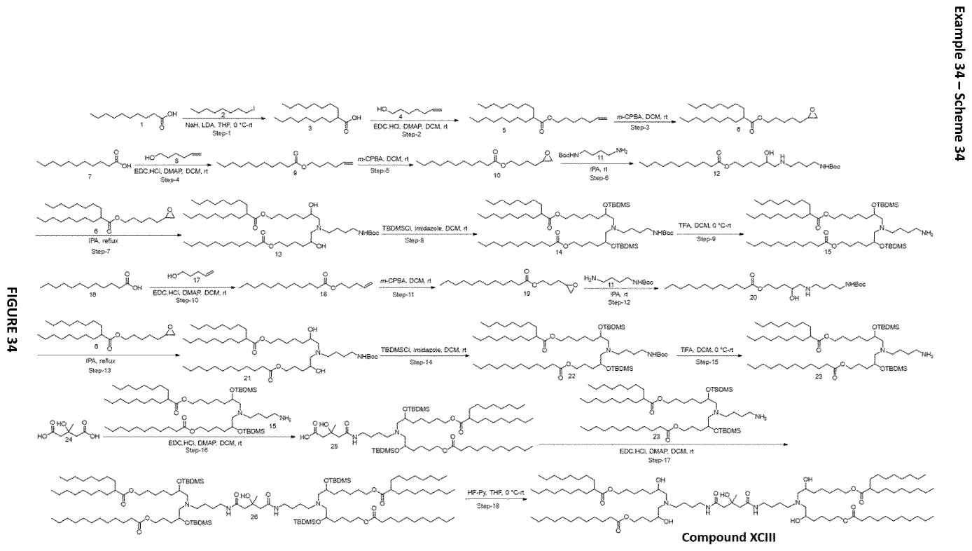

For example, the compounds of the invention may be prepared according to Scheme 34 (as depicted in FIG. 34).

### Intermediate [15] and Intermediate [23]

As depicted in Scheme 34: Synthesis procedure of Intermediate [15] and Intermediate [23] is given in the synthesis for Compound LXXVIII.

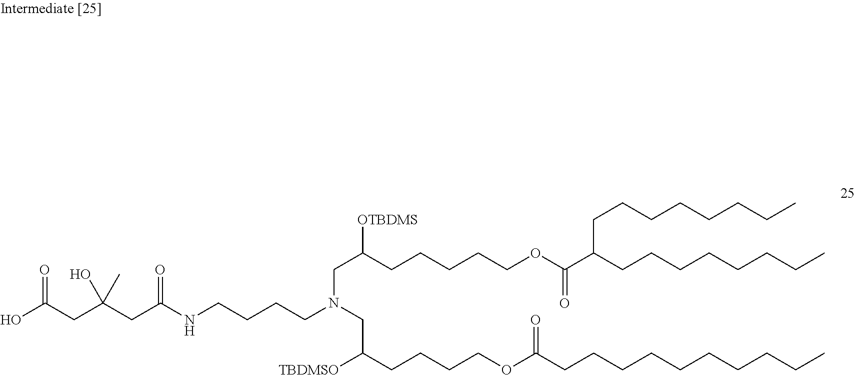 [SMILES: CCCCCCCCCCC(=O)OCCCCC(CN(CCCCNC(=O)CC(C)(O)CC(=O)O)CC(CCCCCOC(=O)C(CCCCCCCC)CCCCCCCC)O[Si](C)(C)C(C)(C)C)O[Si](C)(C)C(C)(C)C]

As depicted in Scheme 34: To a stirred solution of [(carboxymethyl)-N-methylamino]acetic acid [24](0.3 g, 1.85 mmol) in tetrahydrofuran (15 mL) and dimethylformamide (3 mL) was added 1H-1,2,3-benzotriazol-1-yloxytridimethylaminophosphonium hexafluoridophosphate(1-) (818 mg, 1.85 mmol) followed by N-ethylbis(isopropyl)amine (359 mg, 2.78 mmol). The reaction mixture was stirred at RT for 6 hr. The progress of reaction was monitored by TLC and ELSD. After that 6-[(4-aminobutyl){2-[(tert-butyl)bis(methyl)siloxy]-7-(1-octylnonylcarbonyloxy)heptyl}amino]-5-[(tert-butyl)bis(methyl)siloxy]hexyl undecanoate [15](858 mg, 859 μmol) was added into reaction mass. The reaction mixture was stirred at RT for 16 h. The progress of reaction was monitored by ELSD data and TLC. After completion of reaction, reaction mass quenched by water (15 mL) and extracted with DCM (3×25 mL). The organic layers were collected, combined, dried over anhy. sodium sulphate and concentrated under reduced pressure to get crude. The crude was purified by flash column chromatography (SiO₂: 0-10% methanol in DCM to get 4-[N-4-({2-[(tert-butyl)bis(methyl)siloxy]-6-(decylcarbonyloxy)hexyl}{2-[(tert-butyl)bis(methyl)siloxy]-7-(1-octylnonylcarbonyloxy) heptyl}amino)butylcarbamoyl]-3-hydroxy-3-methylbutyric acid [25](1.0 g, 47.39% Yield) as a pale yellow liquid.

### Result:

ELSD analysis: Purity 97.31%, Calculated C₆₄H₁₂₃N₂O₁₀Si₂=1140.91, Observed=1141.45 (m/z, M+H⁺).

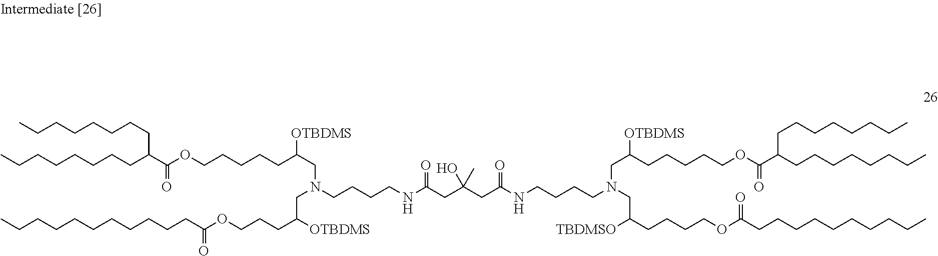 [SMILES: CCCCCCCCCCCC(=O)OCCCC(CN(CCCCNC(=O)CC(C)(O)CC(=O)NCCCCN(CC(CCCCCOC(=O)C(CCCCCCCC)CCCCCCCC)O[Si](C)(C)C(C)(C)C)CC(CCCCOC(=O)CCCCCCCCCC)O[Si](C)(C)C(C)(C)C)CC(CCCCCOC(=O)C(CCCCCCCC)CCCCCCCC)O[Si](C)(C)C(C)(C)C)O[Si](C)(C)C(C)(C)C]

As depicted in Scheme 34: To a stirred solution of 4-[N-4-({2-[(tert-butyl)bis(methyl)siloxy]-6-(decylcarbonyloxy) hexyl}{2-[(tert-butyl)bis(methyl)siloxy]-7-(1-octylnonylcarbonyloxy) heptyl}amino) butylcarbamoyl]-3-hydroxy-3-methylbutyric acid [25](1.0 g, 876 μmol) in dichloromethane (20 mL) were added N,N-dimethyl-4-pyridylamine (53.5 mg, 0.5 eq., 438 μmol) and EDC·HCl (252 mg, 1.31 mmol). The reaction mixture was stirred at RT for 0.5 hr. after that 5-[(4-aminobutyl){2-[(tert-butyl)bis(methyl)siloxy]-7-(1-octylnonylcarbonyloxy) heptyl}amino]-4-[(tert-butyl)bis(methyl)siloxy]pentyl dodecanoate [23](786 mg, 788 μmol). The reaction mixture was stirred at RT for 48 h. The progress of reaction was monitored by ELSD data and TLC. After completion of reaction, reaction mass quenched by water (10 mL) and extracted with DCM (3×25 mL). The organic layers were combined and dried over sodium sulphate and concentrated under reduced pressure to get crude. The crude was purified by flash column chromatography (SiO₂: 0-10% ethyl acetate in hexane) to get 6,26-bis((tert-butyldimethylsilyl)oxy)-8-(2-((tert-butyldimethylsilyl)oxy)-5-(dodecanoyloxy)pentyl)-24-(2-((tert-butyldimethylsilyl)oxy)-6-(undecanoyloxy)hexyl)-16-hydroxy-16-methyl-14,18-dioxo-8,13,19,24-tetraazahentriacontane-1,31-diyl bis(2-octyldecanoate) [26](370 mg, 19.91% Yield) as a pale yellow liquid.

### Result:

ELSD analysis: Purity 95.68%, Calculated C₁₂₂H₂₄₆N₄O₁₅Si₄=2119.77, Observed=1061.15 (m/z, M/2+H⁺).

### Synthesis of Compound XCIII

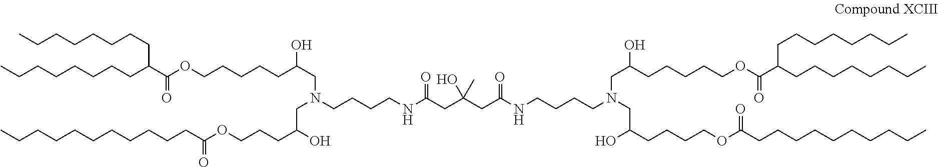 [SMILES: CCCCCCCCCCCC(=O)OCCCC(O)CN(CCCCNC(=O)CC(C)(O)CC(=O)NCCCCN(CC(O)CCCCCOC(=O)C(CCCCCCCC)CCCCCCCC)CC(O)CCCCOC(=O)CCCCCCCCCC)CC(O)CCCCCOC(=O)C(CCCCCCCC)CCCCCCCC]

As depicted in Scheme 34: To a stirred solution of 6,26-bis((tert-butyldimethylsilyl)oxy)-8-(2-((tert-butyldimethylsilyl) oxy)-5-(dodecanoyloxy)pentyl)-24-(2-((tert-butyldimethylsilyl)oxy)-6-(undecanoyloxy) hexyl)-16-hydroxy-16-methyl-14,18-dioxo-8,13,19,24-tetraazahentriacontane-1,31-diyl bis(2-octyldecanoate) [26](0.3 g, 141 μmol) in tetrahydrofuran (3.0 mL) was added hydrogen fluoride pyridine (306 μL, 3.39 mmol) at 0° C., The reaction mixture was stirred at room temperature for 16 h. The progress of reaction was monitored by TLC. After completion of reaction, reaction mixture was diluted with diethyl ether (50 mL) and cold water (2×5 mL). The diethyl ether layer was quenched with cold saturated sodium bicarbonate solution up to pH 8, separated and washed with cold Mili Q water (2×5 mL). The organic layer was dried over Anhy. sodium sulphate, concentrated under reduced pressure to get crude. The crude was purified by flash column chromatography (SiO₂: 0-20% methanol in DCM) to obtain 8-(5-(dodecanoyloxy)-2-hydroxypentyl)-6,16,26-trihydroxy-24-(2-hydroxy-6-(undecanoyloxy) hexyl)-16-methyl-14,18-dioxo-8,13,19,24-tetraazahentriacontane-1,31-diyl bis(2-octyldecanoate) [Compound XCIII](140 mg, 59.48% Yield) as a colourless liquid.

### Results:

¹H-NMR (400 MHz, CDCl₃): δ 7.15-6.75 (m, 2H), 4.09-4.04 (m, 8H), 3.67-2.90 (brm, 10H), 2.62-2.19 (br m, 23H), 1.89-1.73 (m, 2H), 1.72-1.49 (m, 22H), 1.44-1.38 (m, 18H), 1.36-1.18 (m, 83H), 0.87 (t, J=7.2 Hz, 18H).

ELSD analysis: Purity 99.51%, Calculated C₉₈H₁₉₀N₄O₁₅=1663.42, Observed=1664.55 (m/z, M+H⁺).

### Example 35: Synthesis of Compound XCIV

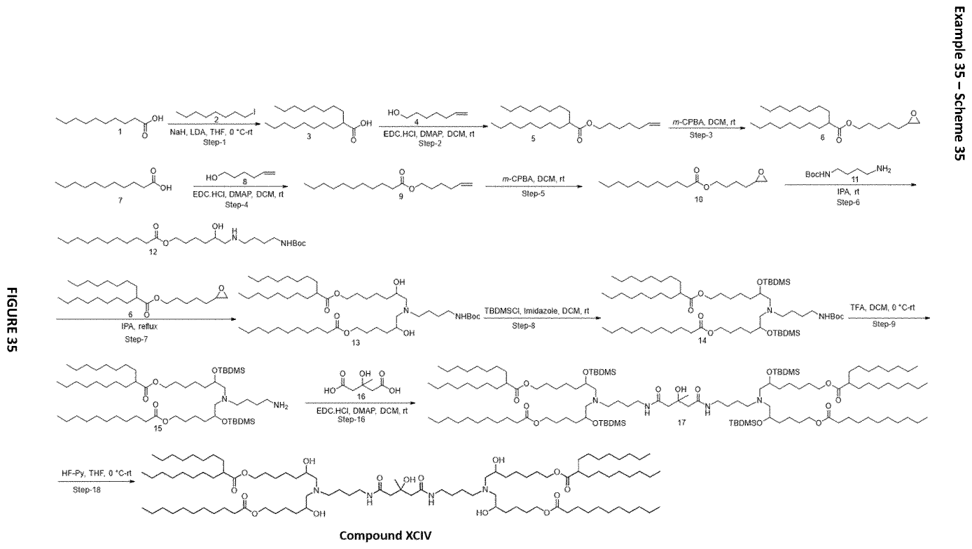

For example, the compounds of the invention may be prepared according to Scheme 35 (as depicted in FIG. 35).

### Intermediate [15]

As depicted in Scheme 35: Synthesis procedure of Intermediate [15] is given in the synthesis for Compound LXXVIII.

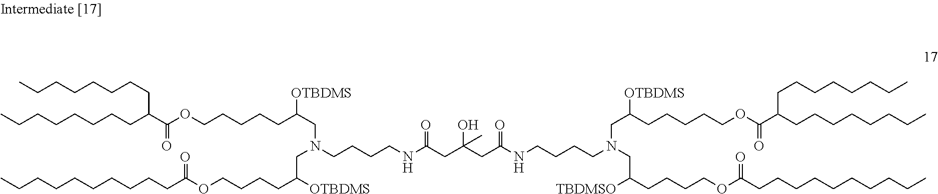 [SMILES: CCCCCCCCCCC(=O)OCCCCC(CN(CCCCNC(=O)CC(C)(O)CC(=O)NCCCCN(CC(CCCCCOC(=O)C(CCCCCCCC)CCCCCCCC)O[Si](C)(C)C(C)(C)C)CC(CCCCOC(=O)CCCCCCCCCC)O[Si](C)(C)C(C)(C)C)CC(CCCCCOC(=O)C(CCCCCCCC)CCCCCCCC)O[Si](C)(C)C(C)(C)C)O[Si](C)(C)C(C)(C)C]

As depicted in Scheme 35: To a stirred solution of 3-hydroxy-3-methylglutaric acid (550 mg, 3.39 mmol) in dichloromethane (50 mL) were added N,N-dimethyl-4-pyridylamine (207 mg, 1.7 mmol), EDC·HCl (975 mg, 5.09 mmol). The reaction mixture was stirred at RT for 15 min and added 6-((4-aminobutyl)(2-((tert-butyldimethylsilyl)oxy)-7-((2-octyldecanoyl)oxy)heptyl)amino)-5-((tert-butyldimethylsilyl)oxy)hexyl undecanoate [15](3.05 g, 3.05 mmol). The reaction mixture was stirred at RT for 16 h. The progress of reaction was monitored by ELSD data and TLC. After completion, reaction mass quenched by water (15 mL) and extracted with DCM (3×25 mL). The organic layers were collected, dried over anhy. sodium sulphate and concentrated under reduced pressure to get crude. The crude was purified by flash column chromatography (SiO₂: 0-10% methanol in DCM) to get 5,25-bis((tert-butyldimethylsilyl)oxy)-7,23-bis(2-((tert-butyldimethylsilyl)oxy)-7-((2-octyldecanoyl)oxy) heptyl)-15-hydroxy-15-methyl-13,17-dioxo-7,12,18,23-tetraazanonacosane-1,29-diyl diundecanoate [17](0.8 g, 11.11% Yield) as a pale yellow liquid.

### Result:

ELSD analysis: Purity 85.57%, Calculated C₁₂₂H₂₄₆N₄O₁₅Si₄=2119.77, Observed=1061.15 (m/z, M/2+H⁺).

### Synthesis of Compound XCIV

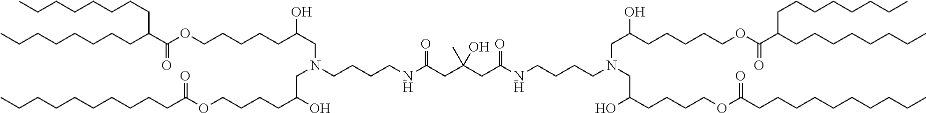 [SMILES: CCCCCCCCCCC(=O)OCCCCC(O)CN(CCCCNC(=O)CC(C)(O)CC(=O)NCCCCN(CC(O)CCCCCOC(=O)C(CCCCCCCC)CCCCCCCC)CC(O)CCCCOC(=O)CCCCCCCCCC)CC(O)CCCCCOC(=O)C(CCCCCCCC)CCCCCCCC]

As depicted in Scheme 35: To a stirred solution of 5,25-bis((tert-butyldimethylsilyl)oxy)-7,23-bis(2-((tert-butyldimethylsilyl)oxy)-7-((2-octyldecanoyl)oxy)heptyl)-15-hydroxy-15-methyl-13,17-dioxo-7,12,18,23-tetraazanonacosane-1,29-diyl diundecanoate [17](0.8 g 0.377 mmol) in tetrahydrofuran (8 mL) was added hydrogen fluoride pyridine (815 μL, 9.05 mmol) at 0° C. The reaction mixture was stirred at room temperature for 16 h. The progress of reaction was monitored by TLC. After completion of the reaction, reaction mixture was quenched with cold saturated sodium bicarbonate solution up to pH 8 and extracted with ethyl acetate (3×10 mL). The organic layer was dried over sodium sulphate, concentrated under reduced pressure to get crude. The crude was purified by column chromatography (SiO₂: 0-10% methanol in DCM) to obtain 8-(5-(dodecanoyloxy)-2-hydroxypentyl)-6,16,26-trihydroxy-24-(2-hydroxy-6-(undecanoyloxy)hexyl)-16-methyl-14,18-dioxo-8,13,19,24-tetraazahentriacontane-1,31-diyl bis(2-octyldecanoate) [Compound XCIV](0.3 g, 47.79% Yield) as a pale yellow liquid.

### Results:

¹H-NMR (400 MHz, CDCl₃): δ 7.18-6.80 (m, 2H), 5.56-5.47 (m, 1H), 4.05 (t, J=6.8 Hz, 8H), 3.75-3.61 (brs, 4H), 3.49-2.98 (m, 4H), 2.72-2.51 (m, 6H), 2.49-2.39 (m, 9H), 2.37-2.26 (m, 9H), 1.661-1.46 (m, 29H), 1.42-1.37 (m, 18H), 1.29-1.13 (m, 82H), 0.87 (t, J=7.2 Hz, 18H).

ELSD analysis: Purity 99.86%, Calculated C₉₈H₁₉₀N₄O₁₅=1663.42, Observed=1664.65 (m/z, M+H⁺).

### Example 36: Synthesis of Compound LXXXI

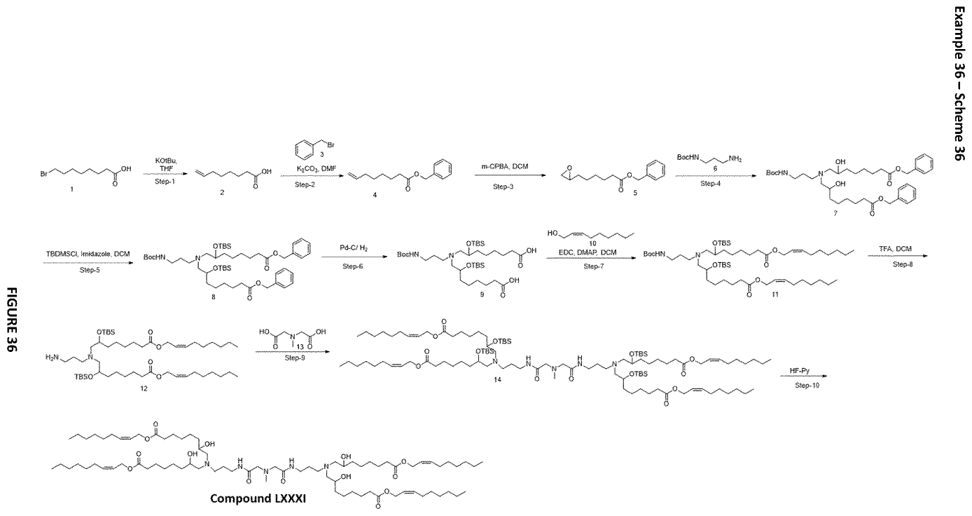

For example, the compounds of the invention may be prepared according to Scheme 36 (as depicted in FIG. 36).

### Intermediate [5]

As depicted in Scheme 36: Synthesis procedure of Intermediate [5] is given in the synthesis for Compound CV.

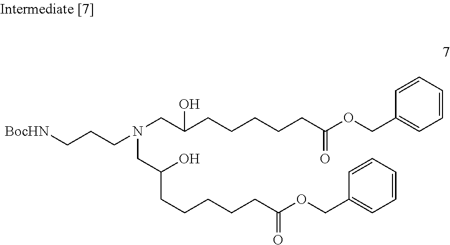 [SMILES: CC(C)(C)OC(=O)NCCCN(CC(O)CCCCCC(=O)OCc1ccccc1)CC(O)CCCCCC(=O)OCc1ccccc1]

As depicted in Scheme 36: To a stirred solution of tert-butyl (3-aminopropyl)carbamate [6](4.0 g, 23 mmol) in isopropanol (40.0 mL) was added benzyl 6-(2-oxiranyl)hexanoate [5](13.1 g, 52.8 mmol). The reaction mixture was stirred at 90° C. for 16 h. The progress of reaction was monitored by ELSD data and TLC. After completion of reaction, reaction mass was concentrated under reduced pressure to get dibenzyl 8,8′-((3-((tert-butoxycarbonyl)amino) propyl)azanediyl)bis(7-hydroxyoctanoate) [7](16.0 g, crude) as a reddish liquid, which was used for next step without further purification.

### Result:

ELSD analysis: Purity 94.12%, Calculated C₃₈H₅₈N₂O₈=670.42, Observed=671.30 (m/z, M+H⁺).

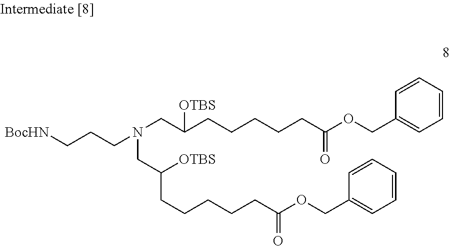 [SMILES: CC(C)(C)OC(=O)NCCCN(CC(CCCCCC(=O)OCc1ccccc1)O[Si](C)(C)C(C)(C)C)CC(CCCCCC(=O)OCc1ccccc1)O[Si](C)(C)C(C)(C)C]

As depicted in Scheme 36: To a stirred solution of dibenzyl 8,8′-((3-((tert-butoxycarbonyl)amino)propyl)azanediyl)bis(7-hydroxyoctanoate) [7](15.5 g, 23.1 mmol) in dichloromethane (200 mL) were added imidazole (25.2 g, 370 mmol), and (tert-butyl)(chloro)bis(methyl)silane (27.9 g, 185 mmol) portion wise. The reaction mixture was stirred at RT for 16 h. The progress of reaction was monitored by TLC/ELSD. After completion of reaction, reaction mixture was diluted with water and extracted with DCM (2×300 mL). The combined organic layer was dried anhydrous sodium sulphate, concentrated under reduced vacuum. The crude was purified by flash column chromatography (SiO₂: 0-30% EtOAc/Heptane) to afford dibenzyl 8,8′-((3-((tert-butoxycarbonyl)amino)propyl)azanediyl)bis(7-((tert-butyldimethylsilyl)oxy)octanoate) [8](16.0 g, 76.99% Yield) as a colourless liquid.

### Result:

ELSD analysis: Purity 91.65%, Calculated C₅₀H₈₆N₂O₈Si₂=898.59, Observed=899.40 (m/z, M+H⁺).

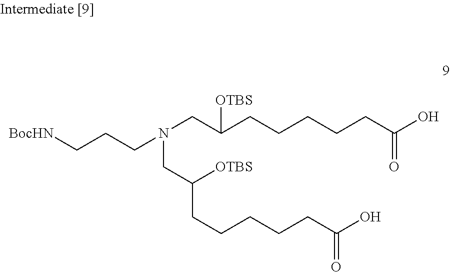 [SMILES: CC(C)(C)OC(=O)NCCCN(CC(CCCCCC(=O)O)O[Si](C)(C)C(C)(C)C)CC(CCCCCC(=O)O)O[Si](C)(C)C(C)(C)C]

As depicted in Scheme 36: To a stirred solution of dibenzyl 8,8′-((3-((tert-butoxycarbonyl)amino)propyl)azanediyl)bis(7-((tert-butyldimethylsilyl)oxy)octanoate) [8](10.0 g, 11.1 mmol) in tetrahydrofuran (50.0 mL), methanol (50.0 mL) was added 10% palladium on carbon (50% wet) (5.0 g, 47 mmol) under nitrogen atmosphere. The reaction mixture was degassed and allowed to stir at room temperature overnight under hydrogen atmosphere (balloon pressure). The progress of reaction was monitored by TLC. After completion of reaction, reaction mixture was filtered through celite and washed with ethyl acetate. The filtrate was concentrated under reduced pressure to get crude compound of 8,8′-((3-((tert-butoxycarbonyl)amino)propyl)azanediyl)bis(7-((tert-butyldimethylsilyl)oxy)octanoic acid) [9](7.0 g, 87.54% Yield) as a colourless liquid.

### Result:

ELSD analysis: Purity 97.83%, Calculated C₃₆H₇₄N₂O₈Si₂=718.50, Observed=719.30 (m/z, M+H⁺).

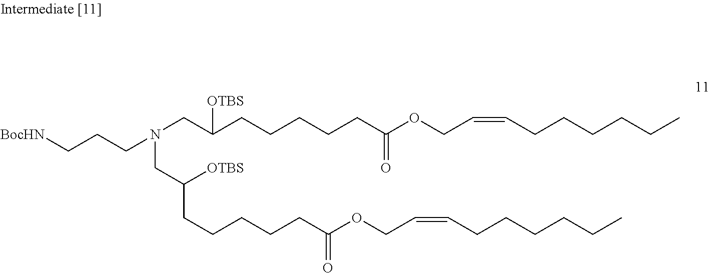 [SMILES: CCCCCC/C=C\COC(=O)CCCCCC(CN(CCCNC(=O)OC(C)(C)C)CC(CCCCCC(=O)OC/C=C\CCCCCC)O[Si](C)(C)C(C)(C)C)O[Si](C)(C)C(C)(C)C]

As depicted in Scheme 36: To a stirred solution of 8,8′-((3-((tert-butoxycarbonyl)amino)propyl)azanediyl)bis(7-((tert-butyldimethylsilyl)oxy)octanoic acid) [9](7.0 g, 9.73 mmol) in dichloromethane (100.0 mL) were added 4-(dimethylamino)pyridin-1-ium (599 mg, 4.87 mmol) and EDC·HCl (2.8 g, 14.6 mmol) at room temperature. The reaction mixture was stirred at RT for 15 min and added (Z)-non-2-en-1-ol (2.91 g, 20.4 mmol). The reaction mixture was stirred at room temperature for 48 h. The progress of reaction was monitored by ELSD data and TLC. After completion of reaction, reaction mixture was quenched with water (50 mL) and extracted with ethyl acetate (2×50 mL). The organic layer was dried over Na₂SO₄ and concentrated under reduced pressure to get crude. The crude was purified by flash column chromatography (SiO2: 40-50% EtOAc/Heptane) to obtain di((Z)-non-2-en-1-yl) 8,8′-((3-((tert-butoxycarbonyl)amino) propyl)azanediyl)bis(7-((tert-butyldimethylsilyl)oxy)octanoate) [11](5.7 g, 48.41% Yield) as a pale yellow liquid.

### Result:

ELSD analysis: Purity 78.80%, Calculated C₅₄H₁₀₆N₂O₈Si₂=966.75, Observed=967.35 (m/z, M+H⁺).

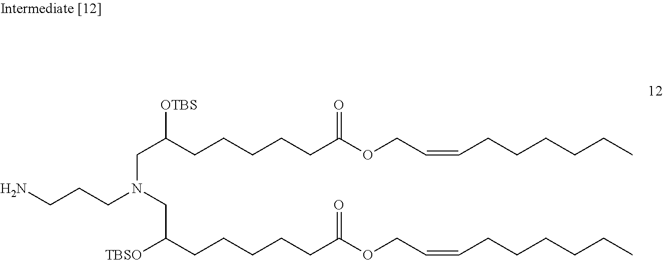 [SMILES: CCCCCC/C=C\COC(=O)CCCCCC(CN(CCCN)CC(CCCCCC(=O)OC/C=C\CCCCCC)O[Si](C)(C)C(C)(C)C)O[Si](C)(C)C(C)(C)C]

As depicted in Scheme 36: To a stirred solution of di((Z)-non-2-en-1-yl) 8,8′-((3-((tert-butoxycarbonyl)amino) propyl)azanediyl)bis(7-((tert-butyldimethylsilyl)oxy)octanoate) [11](4.4 g, 4.55 mmol) in dichloromethane (45.0 mL) was added trifluoroacetic acid (3.48 mL, 45.5 mmol) at cooling condition. The reaction mixture was stirred at RT for 3 h. The progress of reaction was monitored by ELSD data and TLC. After completion of reaction, reaction mass was quenched with ice cold saturated aqueous sodium bicarbonate solution and extracted with ethyl acetate (2×100 mL). The organic layer was dried over anhy. sodium sulphate and concentrated under reduced pressure to afford di((Z)-non-2-en-1-yl) 8,8′-((3-aminopropyl)azanediyl)bis(7-((tert-butyldimethylsilyl)oxy)octanoate) [12](4.0 g, crude) used for next step without further purification.

### Result:

ELSD analysis: Purity 98.47%, Calculated C₄₉H9₃N₂O₆Si₂=866.70, Observed=867.45 (m/z, M+H⁺).

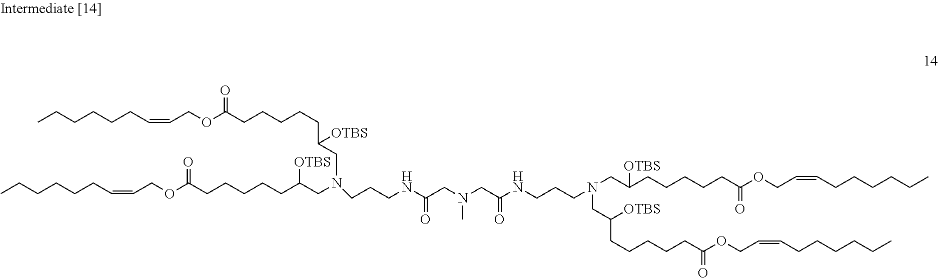 [SMILES: CCCCCC/C=C\COC(=O)CCCCCC(CN(CCCNC(=O)CN(C)CC(=O)NCCCN(CC(CCCCCC(=O)OC/C=C\CCCCCC)O[Si](C)(C)C(C)(C)C)CC(CCCCCC(=O)OC/C=C\CCCCCC)O[Si](C)(C)C(C)(C)C)CC(CCCCCC(=O)OC/C=C\CCCCCC)O[Si](C)(C)C(C)(C)C)O[Si](C)(C)C(C)(C)C]

As depicted in Scheme 36: To a stirred solution of [(carboxymethyl)-N-methylamino]acetic acid [13](646 mg, 4.39 mmol) in dichloromethane (10.0 mL) were added N,N-dimethylpyridin-4-amine (134 mg, 1.1 mmol) and EDC·HCl (631 mg, 3.29 mmol). The reaction mixture was stirred at RT for 15 min and added di((Z)-non-2-en-1-yl) 8,8′-((3-aminopropyl)azanediyl)bis(7-((tert-butyldimethylsilyl)oxy)octanoate) [12](4.0 g, 4.61 mmol). Reaction mixture was stirred at RT for 16 h. The progress of reaction was monitored by ELSD data and TLC. After completion of reaction, water was added into the reaction mixture and extract with DCM (2×50 mL). The organic layer was dried over Na₂SO₄, and concentrated under reduce pressure to get crude. The crude was purified by flash column chromatography (SiO₂: 15-18% Ethyl acetate in Heptane) to afford di((Z)-non-2-en-1-yl) 7,25-bis((tert-butyldimethylsilyl)oxy)-9,23-bis(2-((tert-butyldimethylsilyl)oxy)-8-(((Z)-non-2-en-1-yl)oxy)-8-oxooctyl)-16-methyl-14,18-dioxo-9,13,16,19,23-pentaazahentriacontanedioate [14] (1.6 g, 39.47% Yield) as a colour less liquid.

### Result:

ELSD analysis: Purity 97.95%, Calculated C₁₀₃H₂₀₁N₅O₁₄Si₄=1844.42, Observed=1845.75 (m/z, M+H⁺).

### Synthesis of Compound LXXXI

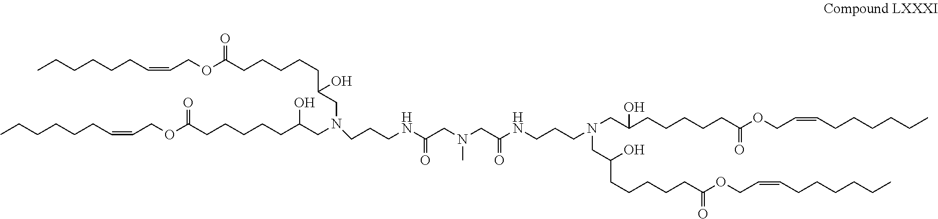 [SMILES: CCCCCC/C=C\COC(=O)CCCCCC(O)CN(CCCNC(=O)CN(C)CC(=O)NCCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC]

As depicted in Scheme 36: To a stirred solution of di((Z)-non-2-en-1-yl) 7,25-bis((tert-butyldimethylsilyl)oxy)-9,23-bis(2-((tert-butyldimethylsilyl)oxy)-8-(((Z)-non-2-en-1-yl)oxy)-8-oxooctyl)-16-methyl-14,18-dioxo-9,13,16,19,23-pentaazahentriacontanedioate [14](1.6 g, 867 μmol), in tetrahydrofuran (16 mL) was cooled to 0° C. and added hydrogen fluoride pyridine (781 μL, 8.67 mmol). The reaction mass was stirred at RT for 16 h. The progress of reaction was monitored by ELSD data and TLC. After completion of reaction, reaction mass was quenched in ice cold saturated aqueous sodium bicarbonate and extracted with EtOAc (2×20 mL). The organic layer was dried over anhy. sodium sulphate and evaporated under reduced pressure to get crude. The crude was purified by flash column chromatography (SiO₂: 2-3% MeOH in DCM) to afford di((Z)-non-2-en-1-yl) 7,25-dihydroxy-9,23-bis(2-hydroxy-8-(((Z)-non-2-en-1-yl)oxy)-8-oxooctyl)-16-methyl-14,18-dioxo-9,13,16,19,23-pentaazahentriacontanedioate [Compound LXXXI](650 mg, 53.99% Yield) as a brownish liquid.

### Results:

¹H-NMR (400 MHz, CDCl₃): δ 7.77-7.70 (m, 2H), 5.67-5.60 (m, 4H), 5.34-5.48 (m, 4H), 4.62 (d, J=6.8 Hz, 8H), 3.65 (m, 4H), 3.46-3.45 (m, 5H), 3.07 (s, 4H), 2.66-2.64 (m, 3H), 2.53-2.50 (m, 2H), 2.46-2.36 (m, 4H), 2.35-2.20 (m, 14H), 2.11-20.6 (m, 8H), 1.68-1.60 (m, 10H), 1.47-1.27 (m, 58H), 0.86 (t, J=7.2 Hz, 12H).

ELSD analysis: Purity 99.73%, Calculated C₇₉H₁₄₅N₅O₁₄=1388.08, Observed=1389.15 (m/z, M+H⁺).

### Example 37: Synthesis of Compound LXXXII

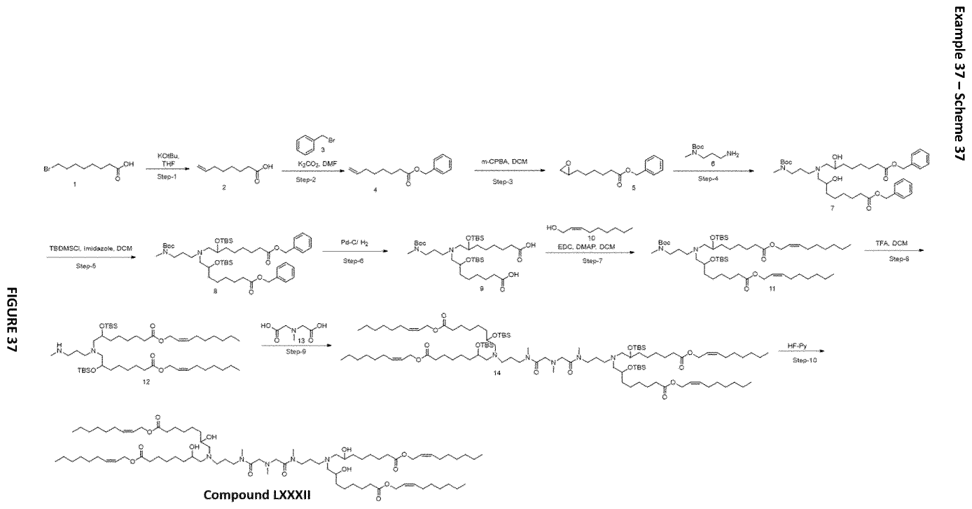

For example, the compounds of the invention may be prepared according to Scheme 37 (as depicted in FIG. 37).

### Intermediate [5]

Synthesis procedure of Intermediate [5] is given in the synthesis for Compound CV.

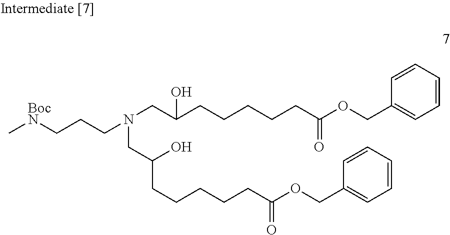 [SMILES: CN(CCCN(CC(O)CCCCCC(=O)OCc1ccccc1)CC(O)CCCCCC(=O)OCc1ccccc1)C(=O)OC(C)(C)C]

As depicted in Scheme 37: To a stirred solution of tert-butyl (3-aminopropyl)(methyl)carbamate [6](4.5 g, 23.9 mmol) in isopropanol (40.0 mL) was added benzyl 6-(2-oxiranyl)hexanoate [5](13.7 g, 55 mmol) and reaction mixture was stirred at 90° C. for 16 h. The progress of reaction was monitored by ELSD data and TLC. After completion of reaction, reaction mass was concentrated under reduced pressure to get crude. The crude of dibenzyl 8,8′-((3-((tert-butoxycarbonyl)(methyl)amino) propyl)azanediyl)bis(7-hydroxyoctanoate) [7](16.4 g, crude) as a reddish liquid used for next step without further purification.

### Result:

ELSD analysis: Purity 92.88%, Calculated C₃₉H₆₀N₂O₈=684.43, Observed=685.35 (m/z, M+H⁺).

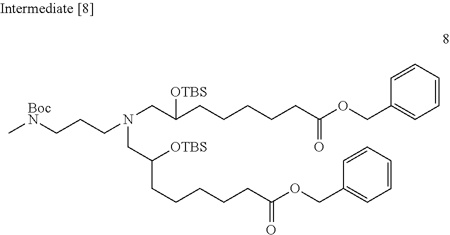 [SMILES: CN(CCCN(CC(CCCCCC(=O)OCc1ccccc1)O[Si](C)(C)C(C)(C)C)CC(CCCCCC(=O)OCc1ccccc1)O[Si](C)(C)C(C)(C)C)C(=O)OC(C)(C)C]

As depicted in Scheme 37: To a stirred solution of dibenzyl 8,8′-((3-((tert-butoxycarbonyl)(methyl)amino) propyl)azanediyl)bis(7-hydroxyoctanoate) [7](16.4 g, 23.9 mmol) in dichloromethane (200 mL) were added imidazole (26.1 g, 383 mmol) and (tert-butyl)(chloro)bis(methyl)silane (28.9 g, 192 mmol) portion wise. The reaction mixture was stirred at RT for 16 h. The progress of reaction was monitored by TLC/ELSD. After completion of reaction, reaction mixture was diluted with water and extracted with DCM (2×300 mL). The combined organic layer was dried anhydrous sodium sulphate, concentrated under reduced vacuum. The crude was purified by flash column chromatography (SiO₂: 0-30% EtOAc/Heptane) to afford dibenzyl 8,8′-((3-((tert-butoxycarbonyl)(methyl)amino)propyl)azanediyl)bis(7-((tert-butyldimethylsilyl)oxy) octanoate) [8] (16.0 g, 59.43% Yield) as a colourless liquid.

### Result:

ELSD analysis: Purity 99.84%, Calculated C₅₁H₃₃N₂OSi₂=912.61, Observed=913.25 (m/z, M+H⁺).

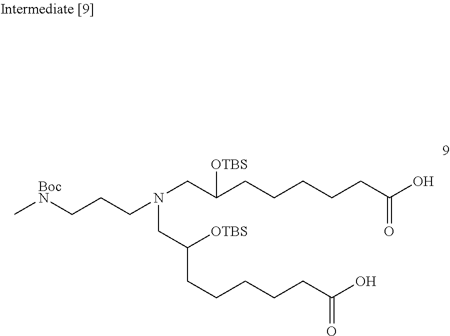 [SMILES: CN(CCCN(CC(CCCCCC(=O)O)O[Si](C)(C)C(C)(C)C)CC(CCCCCC(=O)O)O[Si](C)(C)C(C)(C)C)C(=O)OC(C)(C)C]

As depicted in Scheme 37: To a stirred solution of dibenzyl 8,8′-((3-((tert-butoxycarbonyl)(methyl)amino) propyl)azanediyl)bis(7-((tert-butyldimethylsilyl)oxy)octanoate) [8](10.0 g, 11.1 mmol) in tetrahydrofuran (50 mL), methanol (50 mL) was added 10% palladium on carbon (50% wet) (5.0 g, 47 mmol) under nitrogen atmosphere. The reaction mixture was degassed and allowed to stir at room temperature overnight under hydrogen atmosphere (balloon pressure). The progress of reaction was monitored by TLC. After completion of reaction, reaction mixture was filtered through celite and washed with ethyl acetate. The filtrate was concentrated under reduced pressure to get crude compound of 8,8′-((3-((tert-butoxycarbonyl)(methyl)amino) propyl)azanediyl)bis(7-((tert-butyldimethylsilyl)oxy)octanoic acid) [9](7.5 g, 93.43% Yield) as a colourless liquid.

### Result:

ELSD analysis: Purity 98.80%, Calculated C₃₇H₇₆N₂O₈Si₂=732.51, Observed=733.30 (m/z, M+H⁺).

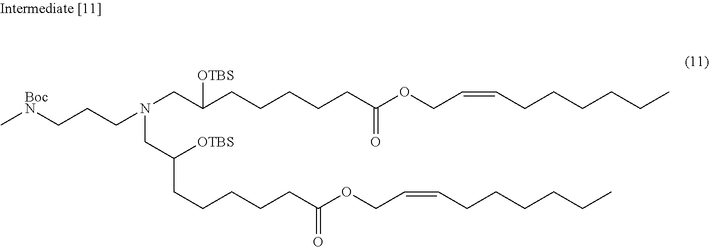 [SMILES: CCCCCC/C=C\COC(=O)CCCCCC(CN(CCCN(C)C(=O)OC(C)(C)C)CC(CCCCCC(=O)OC/C=C\CCCCCC)O[Si](C)(C)C(C)(C)C)O[Si](C)(C)C(C)(C)C]

As depicted in Scheme 37: To a stirred solution of 8,8′-((3-((tert-butoxycarbonyl)(methyl)amino)propyl)azanediyl)bis(7-((tert-butyldimethylsilyl)oxy)octanoic acid) [9](7.5 g, 10.6 mmol) in dichloromethane (100.0 mL) were added 4-(dimethylamino)pyridin-1-ium (664 mg, 5.39 mmol) and EDC·HCl (3.1 g, 16.2 mmol) at room temperature. The reaction mixture was stirred for 10 min and added (Z)-non-2-en-1-ol (2.91 g, 20.4 mmol). The reaction mixture was stirred at room temperature for 48 h. The progress of reaction was monitored by ELSD data and TLC. After completion of reaction, reaction mixture was quenched with water (50 mL) and extracted with ethyl acetate (2×50 mL). The organic layer was dried over Na₂SO₄ and concentrated under reduced pressure to get crude. The crude was purified by flash column chromatography (SiO2: 40-50% EtOAc/Heptane) to obtain di((Z)-non-2-en-1-yl) 8,8′-((3-((tert-butoxycarbonyl)(methyl) amino)propyl)azanediyl)bis(7-((tert-butyldimethylsilyl)oxy)octanoate) [11](6.2 g, 59.46% Yield) as pale yellow liquid.

### Result:

ELSD analysis: Purity 99.80%, Calculated C₅₅H₁₀₃N₂O₃Si₂=980.76, Observed=981.40 (m/z, M+H⁺).

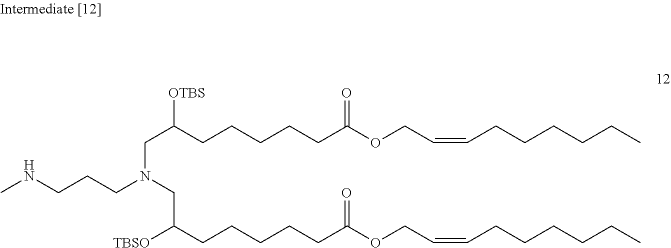 [SMILES: CCCCCC/C=C\COC(=O)CCCCCC(CN(CCCNC)CC(CCCCCC(=O)OC/C=C\CCCCCC)O[Si](C)(C)C(C)(C)C)O[Si](C)(C)C(C)(C)C]

As depicted in Scheme 37: To a stirred solution of di((Z)-non-2-en-1-yl) 8,8′-((3-((tert-butoxycarbonyl) (methyl) amino)propyl)azanediyl)bis(7-((tert-butyldimethylsilyl)oxy) octanoate) [11](4.0 g, 4.07 mmol) in dichloromethane (45.0 mL) was added trifluoroacetic acid (3.12 mL, 40.7 mmol) at cooling, and allow to stirred at RT for 3 h. The progress of reaction was monitored by ELSD data and TLC. After completion, reaction mass was quenched with ice cold saturated aqueous sodium bicarbonate solution and extracted with ethyl acetate (2×100 mL). The organic layers were collect, combined, dried over anhy. sodium sulphate and evaporated under reduced pressure to afford di((Z)-non-2-en-1-yl) 8,8′-((3-(methylamino)propyl)azanediyl)bis(7-((tert-butyldimethylsilyl)oxy)octanoate) [12](3.4 g, crude) used for next step without further purification.

### Result:

ELSD analysis: Purity 99.91%, Calculated C₅₀H₁₀₀N₂O₆Si₂=880.71, Observed=881.40 (m/z, M+H⁺).

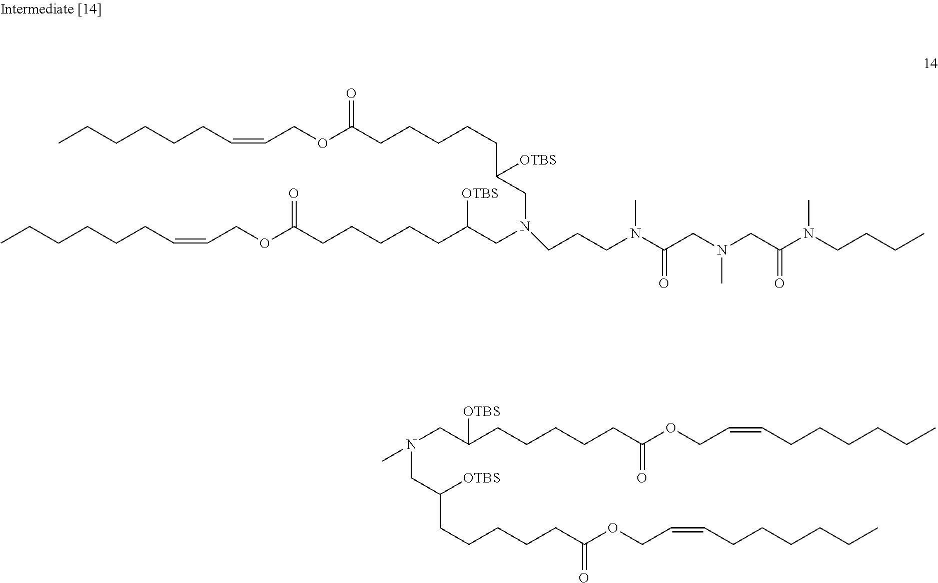

As depicted in Scheme 37: To a stirred solution of [(carboxymethyl)-N-methylamino]acetic acid [13](522 mg, 3.55 mmol) in dichloromethane (10.0 mL) were added N,N-dimethylpyridin-4-amine (120 mg, 986 μmol), and EDC·HCl (567 mg, 2.96 mmol) followed by di((Z)-non-2-en-1-yl) 8,8′-((3-(methylamino)propyl)azanediyl)bis(7-((tert-butyldimethylsilyl)oxy)octanoate) [12](3.48 g, 3.94 mmol). Reaction mixture was stirred at RT for 16 h. The progress of reaction was monitored by ELSD data and TLC. After completion, Water was added into the reaction mixture, and extract with DCM (2×50 mL). The organic layers were collect, combined, dried over Na₂SO₄, and concentrated under reduce pressure, to get crude. The crude was purified by flash column chromatography (SiO₂: 15-18% Ethyl acetate in Heptane) to afford di((Z)-non-2-en-1-yl) 7,25-bis((tert-butyldimethylsilyl)oxy)-9,23-bis(2-((tert-butyldimethylsilyl)oxy)-8-(((Z)-non-2-en-1-yl)oxy)-8-oxooctyl)-13,16,19-trimethyl-14,18-dioxo-9,13,16,19,23-pentaazahentriacontanedioate [14](1.7 g, 47.03% Yield) as pale yellow liquid.

### Result:

ELSD analysis: Purity 99.74%, Calculated C₁₀₅H₂₀₅N₅O₁₄Si₄=1872.46, Observed=1845.75 (m/z, M+H⁺).

### Synthesis of Compound LXXXII

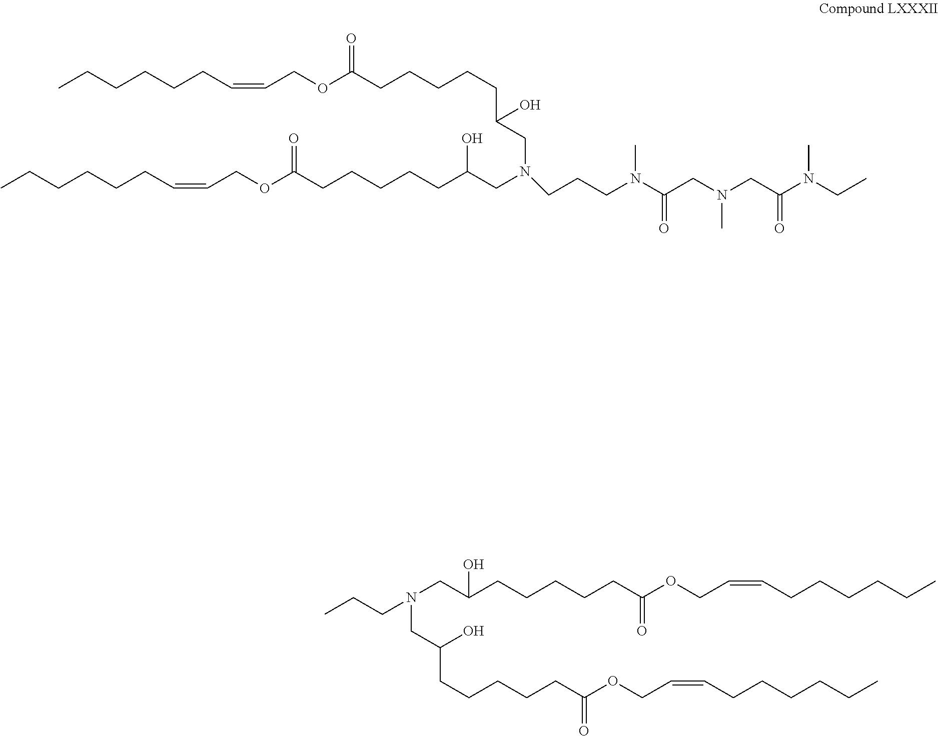

As depicted in Scheme 37: To a stirred solution of di((Z)-non-2-en-1-yl) 7,25-bis((tert-butyldimethylsilyl)oxy)-9,23-bis(2-((tert-butyldimethylsilyl)oxy)-8-(((Z)-non-2-en-1-yl)oxy)-8-oxooctyl)-13,16,19-trimethyl-14,18-dioxo-9,13,16,19,23-pentaazahentriacontanedioate [14](1.7 g, 907 μmol), in tetrahydrofuran (17 mL) was cooled to 0° C., hydrogen fluoride pyridine (781 μL, 9.07 mmol) was added at 0° C. and resultant reaction mass was allowed to stirred at RT for 16 h. The progress of reaction was monitored by ELSD data and TLC. After completion, reaction mass was quenched in ice cold saturated aqueous sodium bicarbonate and extracted with EtOAc (2×20 mL). The organic layers were collect, combined, dried over anhy. sodium sulphate and evaporated under reduced pressure to get crude. The crude was purified by flash column chromatography (SiO₂: 2-3% MeOH in DCM) to afford di((Z)-non-2-en-1-yl) 7,25-dihydroxy-9,23-bis(2-hydroxy-8-(((Z)-non-2-en-1-yl)oxy)-8-oxooctyl)-13,16,19-trimethyl-14,18-dioxo-9,13,16,19,23-pentaazahentriacontanedioate [Compound LXXXII](518 mg, 40.29% Yield) as brownish liquid.

### Results:

¹H-NMR (400 MHz, CDCl₃): δ 5.67-5.60 (m, 4H), 5.34-5.48 (m, 4H), 4.62 (d, J=6.8 Hz, 8H), 3.62-3.53 (m, 4H), 3.51-3.25 (m, 8H), 3.07-2.99 (m, 2H), 2.96-2.95 (m, 2H), 2.89-2.82 (m, 3H), 2.60-2.48 (m, 6H), 2.45-2.34 (m, 8H), 2.31-2.24 (m, 10H), 2.11 (q, J=7.2 Hz, 8H), 1.73-1.60 (m, 12H), 1.53-1.42 (m, 4H), 1.36-1.24 (m, 54H), 0.85 (t, J=7.2 Hz, 12H).

ELSD analysis: Purity 98.54%, Calculated C₈₁H₁₄₉N₅O₁₄=1416.11, Observed=1417.20 (m/z, M+H⁺).

### Example 38: Synthesis of Compound LXXXIII

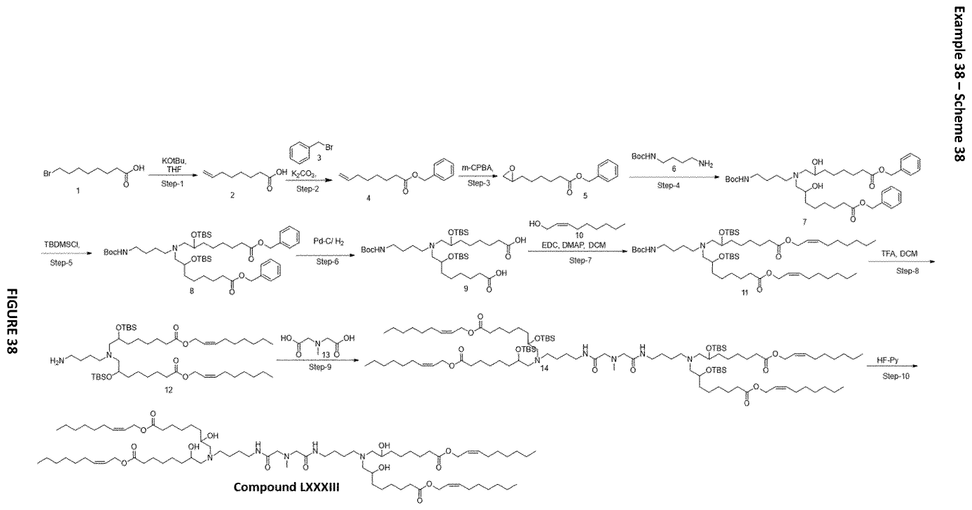

For example, the compounds of the invention may be prepared according to Scheme 38 (as depicted in FIG. 38).

### Intermediate [5]

As depicted in Scheme 38: Synthesis procedure of Intermediate [5] is given in the synthesis for Compound CV.

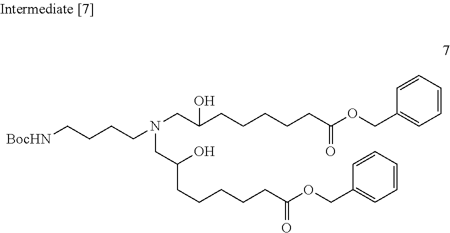 [SMILES: CC(C)(C)OC(=O)NCCCCN(CC(O)CCCCCC(=O)OCc1ccccc1)CC(O)CCCCCC(=O)OCc1ccccc1]

As depicted in Scheme 38: To a stirred solution of tert-butyl (4-aminobutyl)carbamate [6](5.0 g, 26.6 mmol) in isopropanol (50.0 mL) was added benzyl 6-(2-oxiranyl)hexanoate [5](15.2 g, 61.1 mmol), and allow to stirred at 90° C. for 16 h. The progress of reaction was monitored by ELSD data and TLC. After completion, reaction mass was concentrated under reduced pressure to get crude. The crude of dibenzyl 8,8′-((4-((tert-butoxycarbonyl)amino)butyl)azanediyl)bis(7-hydroxyoctanoate) [7](18.0 g, crude) as reddish liquid used for next step without further purification.

### Result:

ELSD analysis: Purity 96.90%, Calculated C₃₉H₆₀N₂O₈=684.43, Observed=685.35 (m/z, M+H⁺).

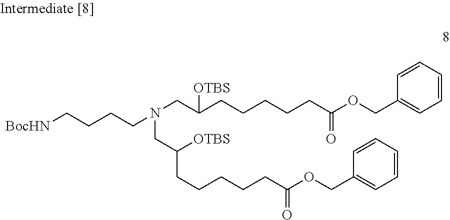 [SMILES: CC(C)(C)OC(=O)NCCCCN(CC(CCCCCC(=O)OCc1ccccc1)O[Si](C)(C)C(C)(C)C)CC(CCCCCC(=O)OCc1ccccc1)O[Si](C)(C)C(C)(C)C]

As depicted in Scheme 38: To a stirred solution of dibenzyl 8,8′-((4-((tert-butoxycarbonyl)amino)butyl)azanediyl)bis(7-hydroxyoctanoate) [7](18.5 g, 27.0 mmol) in dichloromethane (200 mL) were added imidazole (29.4 g, 432 mmol), and (tert-butyl)(chloro)bis(methyl)silane (33.6 g, 216 mmol) portion wise. The reaction mixture was stirred at RT for 16 h. The progress of reaction was monitored by TLC/ELSD. After completion, reaction mixture was diluted with water and extracted with DCM (2×300 mL). The combined organic layer was dried anhydrous sodium sulphate, concentrated under reduced vacuum. The crude was purified by flash column chromatography (SiO₂: 0-30% EtOAc/Heptane) to afford dibenzyl 8,8′-((4-((tert-butoxycarbonyl) amino)butyl)azanediyl)bis(7-((tert-butyldimethylsilyl)oxy)octanoate) [8](21.0 g, 85.11% Yield) as a colourless liquid.

### Result:

ELSD analysis: Purity 97.55%, Calculated C₅₁H₈₈N₂O₈Si₂=912.61, Observed=913.20 (m/z, M+H⁺).

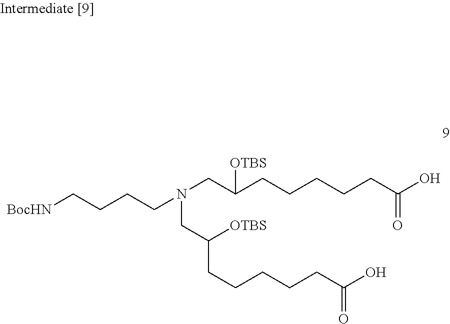 [SMILES: CC(C)(C)OC(=O)NCCCCN(CC(CCCCCC(=O)O)O[Si](C)(C)C(C)(C)C)CC(CCCCCC(=O)O)O[Si](C)(C)C(C)(C)C]

As depicted in Scheme 38: To a stirred solution of dibenzyl 8,8′-((4-((tert-butoxycarbonyl)amino)butyl)azanediyl)bis(7-((tert-butyldimethylsilyl)oxy)octanoate) [8](9.0 g, 9.85 mmol) in tetrahydrofuran (45.0 mL), methanol (45.0 mL) was added 10% palladium on carbon (50% wet) (4.4 g, 41.4 mmol) under nitrogen atmosphere. The reaction mixture was degassed and allowed to stir at room temperature overnight under hydrogen atmosphere (balloon pressure). The progress of reaction was monitored by TLC. After completion, reaction mixture was filtered through celite and washed with ethyl acetate. The filtrate was concentrated under reduced pressure to get crude compound of 8,8′-((4-((tert-butoxycarbonyl)amino)butyl)azanediyl)bis(7-((tert-butyldimethylsilyl)oxy)octanoic acid) [9](7.2 g, 99.0% Yield) as colourless liquid.

### Result:

ELSD analysis: Purity 98.00%, Calculated C₃₇H₇₆N₂O₈Si₂=732.51, Observed=733.25 (m/z, M+H⁺).

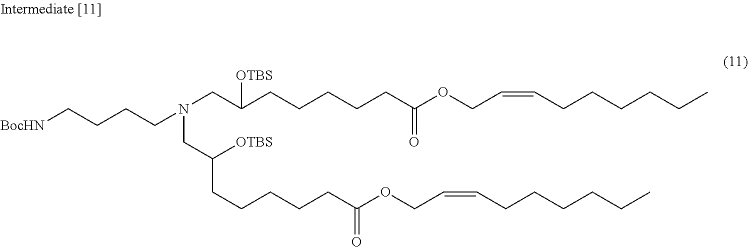 [SMILES: CCCCCC/C=C\COC(=O)CCCCCC(CN(CCCCNC(=O)OC(C)(C)C)CC(CCCCCC(=O)OC/C=C\CCCCCC)O[Si](C)(C)C(C)(C)C)O[Si](C)(C)C(C)(C)C]

As depicted in Scheme 38: To a stirred solution of 8,8′-((4-((tert-butoxycarbonyl)amino)butyl)azanediyl)bis(7-((tert-butyldimethylsilyl)oxy)octanoic acid) [9](5.4 g, 7.37 mmol) in dichloromethane (100.0 mL) were added 4-(dimethylamino)pyridin-1-ium (454 mg, 3.68 mmol) and EDC·HCl (2.12 g, 11.0 mmol) at room temperature. After this (Z)-non-2-en-1-ol (2.62 g, 18.4 mmol) was added into reaction mass and allowed to stirred at room temperature for 48 h. The progress of reaction was monitored by ELSD data and TLC. After completion, reaction mixture was quenched with water (50 mL) and extracted with ethyl acetate (2×50 mL). The organic layers were collect, combined and dried over Na₂SO₄ and concentrated under reduced pressure to get crude. The crude was purified by flash column chromatography (SiO2: 40-50% EtOAc/Heptane) to obtain di((Z)-non-2-en-1-yl)_{8,8}′-((4-((tert-butoxycarbonyl)amino)butyl)azanediyl)bis(7-((tert-butyldimethylsilyl)oxy)octanoate) [11](3.0 g, 41.49% Yield) as pale yellow liquid.

### Result:

ELSD analysis: Purity 99.56%, Calculated C₅₅H₁₀₃N₂O₈Si₂=980.76, Observed=981.40 (m/z, M+H⁺).

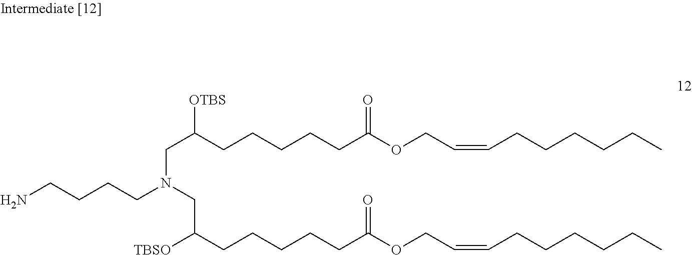 [SMILES: CCCCCC/C=C\COC(=O)CCCCCC(CN(CCCCN)CC(CCCCCC(=O)OC/C=C\CCCCCC)O[Si](C)(C)C(C)(C)C)O[Si](C)(C)C(C)(C)C]

As depicted in Scheme 38: To a stirred solution of di((Z)-non-2-en-1-yl) 8,8′-((4-((tert-butoxycarbonyl) amino)butyl) azanediyl)bis(7-((tert-butyldimethylsilyl)oxy)octanoate) [11](2.8 g, 2.85 mmol) in dichloromethane (30.0 mL) was added trifluoroacetic acid (2.18 mL, 28.5 mmol) at cooling, and allow to stirred at RT for 3 h. The progress of reaction was monitored by ELSD data and TLC. After completion, reaction mass was quenched with ice cold saturated aqueous sodium bicarbonate solution and extracted with ethyl acetate (2×100 mL). The organic layers were collect, combined, dried over anhy. sodium sulphate and evaporated under reduced pressure to afford di((Z)-non-2-en-1-yl) 8,8′-((4-aminobutyl)azanediyl)bis(7-((tert-butyldimethylsilyl) oxy)octanoate) [12](2.1 g, 835.17% Yield) used for next step without further purification.

### Result:

ELSD analysis: Purity 99.56%, Calculated C₅₀H₁₀₀N₂O₆Si₂=880.71, Observed=881.40 (m/z, M+H⁺).

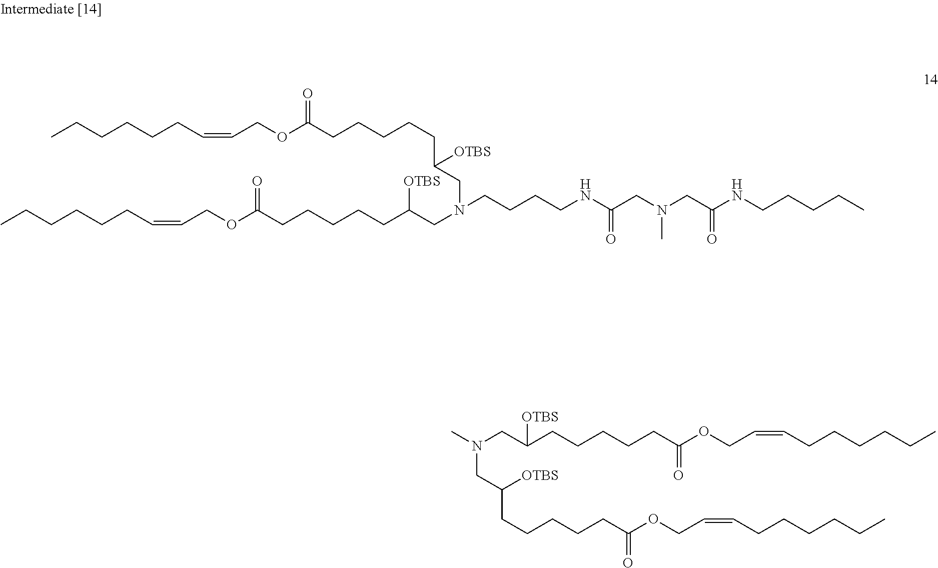

As depicted in Scheme 38: To a stirred solution of [(carboxymethyl)-N-methylamino]acetic acid [13](160 mg, 1.09 mmol) in dichloromethane (5.48 mL) were added N,N-dimethylpyridin-4-amine (66.4 mg, 544 μmol), and EDC·HCl (313 mg, 1.63 mmol) Followed by di((Z)-non-2-en-1-yl) 8,8′-((4-aminobutyl)azanediyl)bis(7-((tert-butyldimethylsilyl)oxy)octanoate) [12](2.1 g, 2.38 mmol), allowed to stirred at RT for 16 h under nitrogen atmosphere. The progress of reaction was monitored by TLC. After completion, reaction mixture was diluted with DCM (50 mL) and washed with brine solution (20 mL) followed by water (20 mL). The organic layers were combined, dried over sodium sulphate, concentrated under reduced pressure. The crude was purified by column chromatography (SiO₂:0-20% Ethyl acetate in Heptane) to afford di((Z)-non-2-en-1-yl) 7,27-bis((tert-butyldimethylsilyl)oxy)-9,25-bis(2-((tert-butyldimethylsilyl) oxy)-8-(((Z)-non-2-en-1-yl)oxy)-8-oxooctyl)-17-methyl-15,19-dioxo-9,14,17,20,25-pentaazatritriacontanedioate [14](1.1 g, 53.97% Yield) as a colour less liquid.

### Results:

ELSD analysis: Purity 99.67%, Calculated C₁₀₅H₂₀₅N₅O₁₄Si₄=1872.46, Observed=1873.75 (m/z, M+H⁺).

### Synthesis of Compound LXXXIII

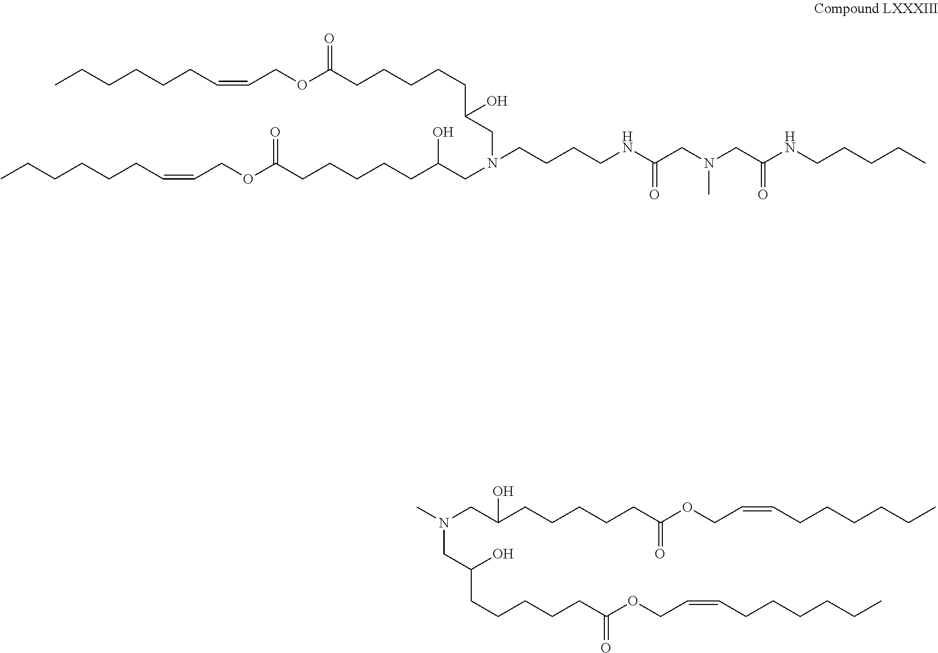

As depicted in Scheme 38: To a stirred solution of di((Z)-non-2-en-1-yl) 7,27-bis((tert-butyldimethylsilyl)oxy)-9,25-bis(2-((tert-butyldimethylsilyl)oxy)-8-(((Z)-non-2-en-1-yl)oxy)-8-oxooctyl)-17-methyl-15,19-dioxo-9,14,17,20,25-pentaazatritriacontanedioate [14](1.1 g, 587 μmol), in tetrahydrofuran (12.0 mL) was cooled to 0° C. Hydrogen fluoride pyridine (529 μL, 5.87 mmol) was added at 0° C., and resultant reaction mass was allowed to stirred at RT for 16 h. The progress of reaction was monitored by ELSD data and TLC. After completion, reaction mass was quenched in ice cold saturated aqueous sodium bicarbonate and extracted with EtOAc (2×20 mL). The organic layers were collect, combined, dried over anhy. sodium sulphate and evaporated under reduced pressure to get crude. The crude was purified by flash column chromatography (SiO2: 2-3% MeOH in DCM) to obtain di((Z)-non-2-en-1-yl) 7,27-dihydroxy-9,25-bis(2-hydroxy-8-(((Z)-non-2-en-1-yl)oxy)-8-oxooctyl)-17-methyl-15,19-dioxo-9,14,17,20,25-pentaazatritriacontanedioate (680 mg, 81.75% Yield) as brownish liquid.

### Results:

¹H-NMR (400 MHz, CDCl3)-δ 8.03-8.01 (m, 2H), 5.63-5.57 (m, 4H), 5.50-5.46 (m, 4H), 4.56-4.54 (d, J=6.8 Hz, 8H), 4.37-4.16 (m, 4H), 3.47-3.38 (m, 4H), 3.12-3.06 (m, 4H), 2.95 (m, 4H), 2.47-2.32 (m, 6H), 2.29-2.21 (m, 12H), 2.19 (s, 3H), 2.08-2.01 (m, 8H), 1.52-1.47 (m, 8H), 1.36-1.17 (m, 66H), 0.87-0.83 (t, J=7.2 Hz, 12H).

ELSD analysis: Purity 99.89%, Calculated C₃₁H₁₄₉N₄O₁₅=1416.11, Observed=1416.55 (m/z, M+H⁺).

### Example 39: Synthesis of Compound XCV

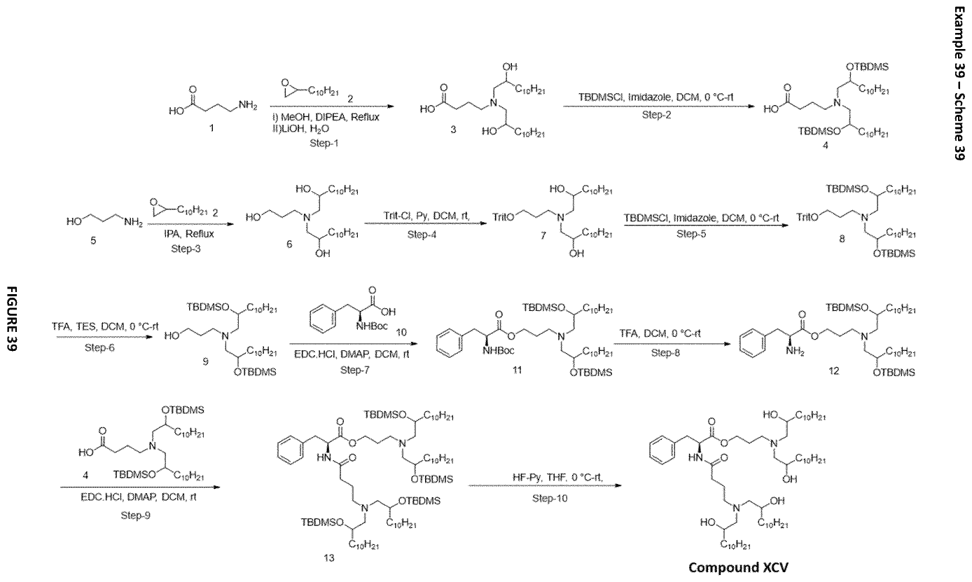

For example, the compounds of the invention may be prepared according to Scheme 39 (as depicted in FIG. 39).

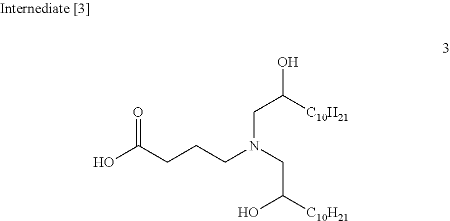

As depicted in Scheme 39: To stirred a solution of 4-aminobutanoic acid [1](5 g, 48.5 mmol) and 2-decyloxirane [2](22.3 g, 121 mmol) in methanol (100 mL) was added bis(propan-2-yl)amine (3.48 g, 34.4 mmol) and heated under nitrogen atmosphere at 95° C. for 20 h. The progress of reaction was monitored by ELSD/TLC (SM was consumed). Reaction mass was cooled to RT. Added lithium(1+) hydroxide (2.32 g, 97 mmol) and water (25.0 mL). Reaction mixture was allowed to stir for 4 h at RT. Progress of reaction mixture was monitored by TLC/ELSD. On reaction completion, excess of methanol was removed under reduced pressure. Residue was acidified with 1N HCl up to pH-3. extract with ethyl acetate (2×50 mL). The organic layers were combined, dried over anhydrous sodium sulphate, concentrated. The crude was purified by flash column chromatography (SiO₂: 0-20% methanol in dichloromethane) to obtain the 4-[bis(2-hydroxydodecyl)amino]butanoic acid [3] (15 g, 65.58% Yield) as a white solid.

### Results:

ELSD analysis: Purity 99.79%, Calculated C₂₈H₅₇NO₄=471.43, Observed=472.40 (m/z, M+H⁺).

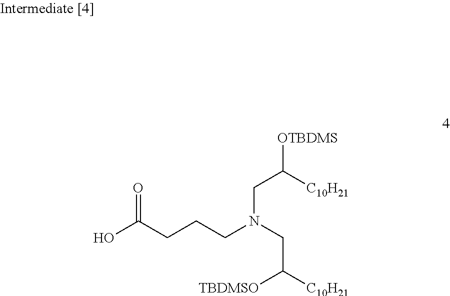

As depicted in Scheme 39: To a stirred solution of 4-[bis(2-hydroxydodecyl)amino]butanoic acid [3](15 g, 31.8 mmol) in dichloromethane (300 mL), were added 1H-imidazole (13 g, 191 mmol) followed by tert-butyl(chloro)dimethylsilane (14.4 g, 95.4 mmol) and allowed reaction mixture for stirring at room temperature for 16 h. The progress of reaction was monitored by TLC/ELSD (starting material was consumed). After completion, the reaction mixture was quenched by water/brine (200 mL) and extracted with dichloromethane (2×500 mL). The combined organic layer was dried over anhydrous sodium sulphate and concentrated under reduced pressure to get crude product. The crude was purified over silica by column chromatography (SiO₂: 0-40% EtOAC/Hexane) to obtain 4-[5,9-bis(decyl)-2,2,3,3,11,11,12,12-octamethyl-4,10-dioxa-7-aza-3,11-disilatridecan-7-yl]butanoic acid [4](18 g, 80.84% Yield) as light green liquid.

### Results:

ELSD analysis: Purity 99.94%, Calculated C₄₀H₈₅NO₄Si₂=699.60, Observed=700.65 (m/z, M+H⁺).

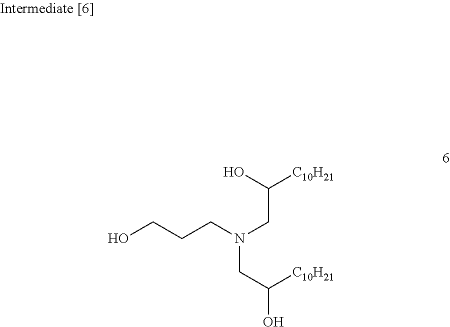

As depicted in Scheme 39: A mixture of 3-aminopropan-1-ol [5](7.5 g, 99.9 mmol) and 2-decyloxirane [2](40.5 g, 220 mmol) and in isopropanol (100 mL) was heated under a nitrogen atmosphere to 95° C. for 20 h. The progress of the reaction was monitored by ELSD/TLC (SM was consumed). The reaction mixture was concentrated, and the crude was purified by flash column chromatography (SiO2: 0-20% methanol in dichloromethane) to obtain the desired 1-[(2-hydroxydodecyl)(3-hydroxypropyl)amino]dodecan-2-ol [6](35.0 g, 79.0%, Yield) as off white solid.

### Results:

ELSD analysis: Purity 99.88%, Calculated C₂₇H₅₇NO₃=443.43, Observed=444.65 (m/z, M+H⁺).

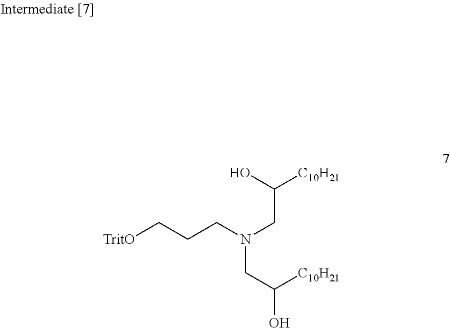

As depicted in Scheme 39: To a stirred solution of 1-[(2-hydroxydodecyl)(3-hydroxypropyl)amino]dodecan-2-ol [6](35.0 g, 78.9 mmol) in dichloromethane (700 mL), was added pyridine (19.1 mL, 237 mmol) followed by (chlorodiphenylmethyl)benzene (24.2 g, 86.8 mmol) and the reaction was stirred at room temperature for 16 h. The progress of reaction was monitored by ELSD/TLC (SM was consumed). After completion, reaction mixture was wash with ice cold water (200.0 mL), and saturated brine solution. The organic layer was dried over sodium sulphate, filtered and concentrated under reduced pressure to give crude compound. The crude was purified by flash column chromatography (SiO₂: 5-6% Methanol in dichloromethane), to obtain the desired 1-[(2-hydroxydodecyl)[3-(triphenylmethoxy)propyl]amino]dodecan-2-ol [7](44.0 g, 81.33%, Yield) as a light green oil.

### Results:

ELSD analysis: Purity 99.13%, Calculated C₄₆H₇₁NO₃=685.54, Observed=686.40 (m/z, M+H⁺).

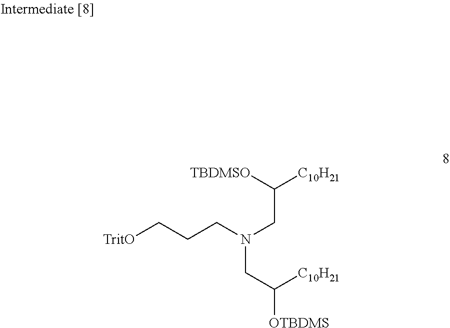

As depicted in Scheme 39: To a stirred solution of 1-[(2-hydroxydodecyl)[3-(triphenylmethoxy)propyl]amino]dodecan-2-ol [7](44.0 g, 64.1 mmol) in dichloromethane (1.0 L), 1H-imidazole (69.9 g, 1.03 mol) and tert-butyl(chloro)dimethylsilane (77.3 g, 513 mmol) was added successively under inert atmosphere. The resultant reaction mass was stirred at RT for 16 h. The progress of reaction was monitor with TLC/ELSD. After completion, reaction mixture was quench with ice cold water (300.0 mL), and the organic layer was separated. The organic layer was wash with saturated brine solution, and dried over sodium sulphate, filtered and concentrated under reduced pressure to give crude compound. The crude was purified by flash column chromatography (0-30% Ethyl acetate in heptane) to afford 5,9-bis(decyl)-2,2,3,3,11,11,12,12-octamethyl-7-[3-(triphenylmethoxy)propyl]-4,10-dioxa-7-aza-3,11-disilatridecane [8](45.0 g, 76.72% Yield) as a colourless liquid.

### Result:

ELSD analysis: Purity 97.97%, Calculated C₅₈H₉₉NO₃Si₂=913.72, Observed=914.50 (m/z, M+H⁺).

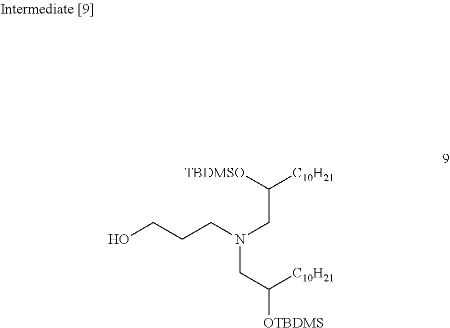

As depicted in Scheme 39: To a stirred solution of [3-(trityloxy)propyl]bis{2-[(tert-butyl)bis(methyl)siloxy]dodecyl}amine [8](25 g, 27.3 mmol) in dichloromethane (250 mL) was added triethylsilane (9.75 mL, 65.6 mmol) at 0° C. followed by the addition of trifluoroacetic acid (12.6 mL, 164 mmol) dropwise under inert atmosphere. The reaction was stirred at RT for 4 h. The progress of reaction was monitor by TLC. After completion, reaction mixture was quenched with saturated aq. NaHCO₃ solution up pH 8. The compound was extracted with DCM (2×500 mL). The combined organic layer was dried over anhy. Na₂SO₄, filtered and evaporated to get crude compound. The crude compound was purified by flash column chromatography (SiO₂: 10-30% Ethyl acetate in Heptane) to afford 3-(bis{2-[(tert-butyl)bis(methyl)siloxy]dodecyl}amino)-1-propanol [9](15.0 g, 81.63% Yield) as a colourless liquid

### Result:

ELSD analysis: Purity 99.38%, Calculated C₃₉H₈₅NO₃Si₂=671.61, Observed=672.30 (m/z, M+H⁺).

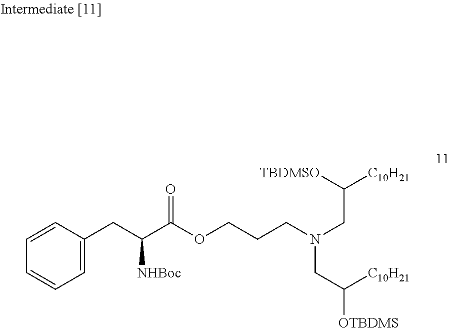

As depicted in Scheme 39: To a stirred solution of (tert-butoxycarbonyl)-L-phenylalanine [10](0.5 g, 1.88 mmol) in dichloromethane (50 mL), were added N,N-dimethyl-4-pyridylamine (230 mg, 1.88 mmol) and EDC·HCl (723 mg, 3.77 mmol) followed by of 3-(bis{2-[(tert-butyl)bis(methyl) siloxy]dodecyl}amino)-1-propanol [9](1.27 g, 1.88 mmol) at RT for 16 h. The progress of reaction was monitored by TLC. After completion, reaction mass was diluted with brine solution (20 mL) and extracted with DCM (2×100 mL). The combined organic layers were dried over anhydrous sodium sulphate and concentrated under reduced pressure to get crude. The crude was the purified over silica (0-20% EtOAc/Hexane) to get 3-(bis(2-((tert-butyldimethylsilyl)oxy)dodecyl)amino)propyl (tert-butoxycarbonyl)-L-phenylalaninate [11](0.9 g 51.93% Yield) as colourless viscous.

### Results:

ELSD analysis: Purity 99.58%, Calculated C₅₃H₁₀₂N₂O₆Si₂=918.73, Observed=919.65 (m/z, M+H⁺).

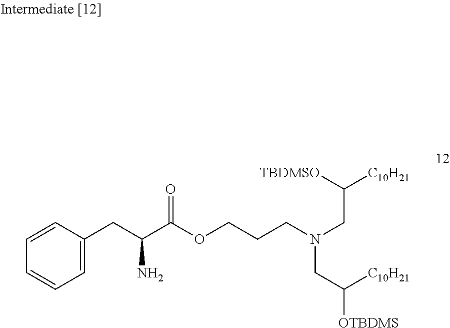

As depicted in Scheme 39: To a stirred solution of 3-(bis(2-((tert-butyldimethylsilyl)oxy)dodecyl)amino)propyl (tert-butoxycarbonyl)-L-phenylalaninate [11](0.9 g, 979 μmol) in dichloromethane (15 mL) was added trifluoroacetic acid (749 μL, 9.79 mmol) at 0° C. for 4 h. The progress of reaction was monitored by TLC/ELSD. After completion, reaction mixture was quenched with saturated solution of sodium bicarbonate (make pH 8-9) and extracted with ethyl acetate (2×50 mL). The combined organic layer was dried over anhy. sodium sulphate, and concentrated under reduced pressure to get 3-(bis(2-((tert-butyldimethylsilyl)oxy)dodecyl)amino)propyl L-phenylalaninate [12](0.780 g, 97.25% Yield) as pale yellow liquid.

### Results:

ELSD analysis: Purity 99.07%, Calculated C₄₃H₉₄N₂O₄Si₂=818.68, Observed=819.60 (m/z, M+H⁺).

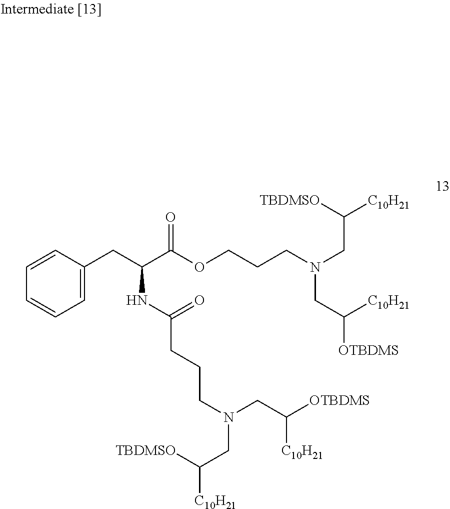

As depicted in Scheme 39: To a stirred solution 4-(bis(2-((tert-butyldimethylsilyl)oxy)dodecyl)amino)butanoic acid [4](0.6 g, 857 μmol) in dichloromethane (20 mL) were added N,N-dimethyl-4-pyridylamine (105 mg, 857 μmol) and EDC·HCl (329 mg, 1.71 mmol) followed by 3-(bis(2-((tert-butyldimethylsilyl)oxy)dodecyl)amino)propyl L-phenylalaninate [12](772 mg, 942 μmol). Allow the reaction mixture to stir at room temperature for 16 h. The progress of reaction was monitored by TLC. After completion of reactin, the reaction mass was diluted with saturated brine solution (2×10 mL), and extracted with DCM (2×25 mL). The combined organic layer was dried over anhydrous sodium sulphate and concentrated under reduced pressure to get crude. The crude was purified by flash column chromatography (SiO₂: 0-20% EtOAc/Heptane) to get 3-(bis(2-((tert-butyldimethylsilyl)oxy)dodecyl)amino)propyl (4-(bis(2-((tert-butyldimethylsilyl)oxy)dodecyl)amino)butanoyl)-L-phenylalaninate [13](580 mg 45.07% Yield) as pale yellow liquid.

### Results:

ELSD analysis: Purity 99.76%, Calculated C₃₃H₁₇₇N₃O₇Si₄=1500.27, Observed=1501.05 (m/z, M+H⁺).

### Synthesis of Compound XCV

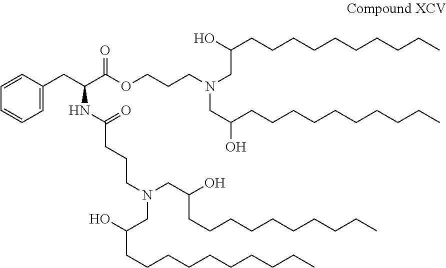 [SMILES: CCCCCCCCCCC(O)CN(CCCOC(=O)[C@H](Cc1ccccc1)NC(=O)CCCN(CC(O)CCCCCCCCCC)CC(O)CCCCCCCCCC)CC(O)CCCCCCCCCC]

As depicted in Scheme 39: To a stirred solution of 3-(bis{2-[(tert-butyl)bis(methyl)siloxy]dodecyl}amino)propyl (S)-2-[4-(bis{2-[(tert-butyl)bis(methyl)siloxy]dodecyl}amino)butyrylamino]-3-phenylpropionate [13](550 mg, 366 μmol) in tetrahydrofuran (5 mL) was added hydrogen fluoride pyridine (1.04 mL, 8.06 mmol) at 0° C. under inert atmosphere for 16 h. The resultant reaction mass was allowed to stirred at RT for 16 h. The progress of reaction was monitored by TLC. After completion, reaction mixture was quench by saturated sodium bicarbonate solution up to pH 8 and extracted with ethyl acetate (3×50 mL). The organic layers were combine, dried over anhydride sodium sulphate, filtered and concentrated under reduced pressure to get crude. The crude was purified by flash column chromatography (SiO₂: 0-5% methanol in dichloromethane) to obtain 3-[bis(2-hydroxydodecyl)amino]propyl (S)-2-{4-[bis(2-hydroxydodecyl)amino]butyrylamino}-3-phenylpropionate [Compound XCV](280 mg, 73.18% Yield) as colourless liquid.

### Results:

¹H-NMR (400 MHz, CDCl3)-δ 8.22-8.17 (m, 1H), 7.28-7.24 (m, 2H), 7.20-7.13 (m, 3H), 4.44-4.39 (m, 1H), 4.28-4.10 (m, 4H), 4.07-3.97 (m, 2H), 3.48-3.35 (m, 4H), 3.02-2.97 (m, 1H), 2.90-2.84 (m, 1H), 2.40-2.18 (m, 10H), 2.10-2.00 (m, 2H), 1.66-1.56 (m, 2H), 1.55-1.45 (m, 2H), 1.40-1.30 (m, 8H), 1.30-1.16 (m, 66H), 0.87-0.83 (t, J=7.2 Hz, 12H).

ELSD analysis: Purity 99.10%, Calculated C₆₄H₁₂₁N₃O₇=1043.92, Observed=1044.75 (m/z, M+H⁺).

### Example 40: Synthesis of Compound XCVI

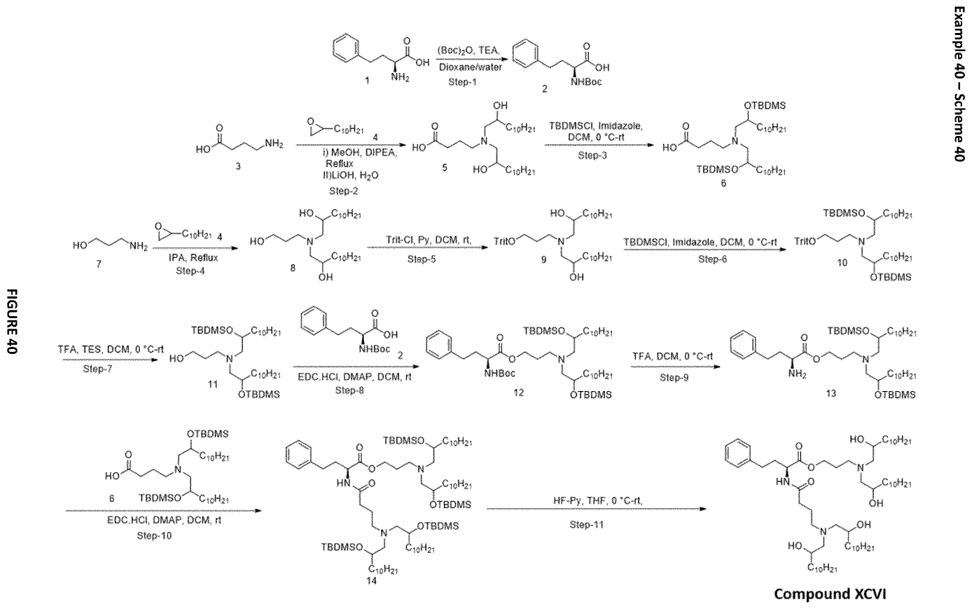

For example, the compounds of the invention may be prepared according to Scheme 40 (as depicted in FIG. 40).

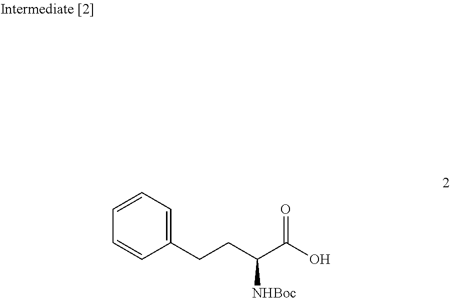 [SMILES: CC(C)(C)OC(=O)N[C@@H](CCc1ccccc1)C(=O)O]

As depicted in Scheme 40: To a stirred solution of (S)-2-amino-4-phenylbutanoic acid [1](0.5 g, 2.79 mmol) in 1,4-dioxane (10 mL) and water (10 mL), were added N-ethylbis(isopropyl)amine (1.94 mL, 11.2 mmol) followed by Boc anhydride (705 μL, 3.07 mmol) at 0° C., allow to stir for stir at room temperature for 16 h. The progress of reaction was monitored by ELSD/TLC. After completion, reaction mass was diluted with water and extracted with ethyl acetate (100 mL). The organic layer was dried over anhydrous sodium sulphate and concentrated under reduced pressure to get crude. The crude was purified by flash column chromatography (SiO₂: 0-80% EtOAc/Hexane) to get (S)-2-((tert-butoxycarbonyl)amino)-4-phenylbutanoic acid [2](0.7 g 89.82% Yield) as colourless liquid.

### Results:

¹H NMR (400 MHz, CDCl3): δ 7.35-7.26 (m, 2H), 7.25-7.16 (m, 3H), 5.16-4.90 (m, 1H), 4.41-4.30 (m, 1H), 2.80-2.65 (t, J=7.6 Hz, 2H), 2.24-2.12 (m, 1H), 2.04-1.97 (m, 1H), 1.47 (s, 9H).

### Intermediate [6]

As depicted in Scheme 40: Synthesis procedure of Intermediate [6] is given in the synthesis for Compound XCV.

### Intermediate [11]

As depicted in Scheme 40: Synthesis procedure of Intermediate [11] is given in the synthesis for Compound XCV.

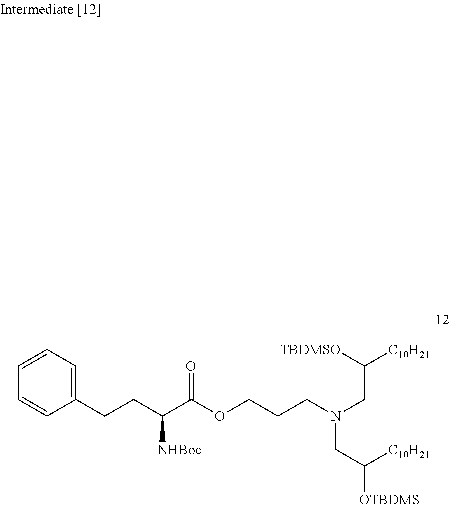

As depicted in Scheme 40: To a stirred solution of (S)-2-((tert-butoxycarbonyl)amino)-4-phenylbutanoic acid [2](0.7 g, 2.51 mmol) in dichloromethane (20 mL, 312 mmol), were added N,N-dimethyl-4-pyridylamine (306 mg, 2.51 mmol) and EDC·HCl (961 mg, 5.01 mmol). Followed by 3-(bis{2-[(tert-butyl)bis(methyl)siloxy]dodecyl}amino)-1-propanol [11](1.68 g, 2.51 mmol) at room temperature for 16 h. The progress of reaction was monitored by ELSD/TLC. After completion, reaction mass diluted with water (10 mL) and extracted with DCM (2×50 mL). The combined organic layers were dried over anhydrous sodium sulphate and concentrated under reduced pressure to get crude. The crude was purified by flash column chromatography (SiO₂: 0-10% EtOAc/Hexane) to get the desired product 3-(bis(2-((tert-butyldimethylsilyl)oxy) dodecyl) amino)propyl (2S)-2-((tert-butoxycarbonyl)amino)-4-phenylbutanoate [12](0.7 g 29.92% Yield) as pale yellow semi solid.

### Results:

ELSD analysis: Purity 99.17%, Calculated C₅₄H₁₀₄N₂O₆Si₂=932.74, Observed=933.60 (m/z, M+H⁺).

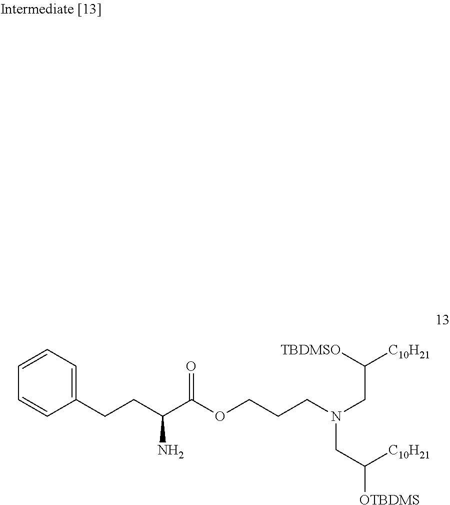

As depicted in Scheme 40: To a stirred solution of 3-(bis(2-((tert-butyldimethylsilyl)oxy)dodecyl)amino)propyl (2S)-2-((tert-butoxycarbonyl)amino)-4-phenylbutanoate [12](0.7 g, 750 μmol) in dichloromethane (10 mL), was added trifluoroacetic acid (574 μL, 7.5 mmol) drop by drop at 0° C., allowed to stir at RT for 4 h. The progress of reaction was monitored by ELSD/TLC. After completion, reaction mass was evaporated under reduced pressure by making azeotrope with Diethyl ether 2 to 3 times to remove the excess amount of TFA. After that reaction mass was diluted with DCM and TFA was quenched with triethyl amine by making pH 8-9 to obtain crude of 3-(bis(2-((tert-butyldimethylsilyl)oxy)dodecyl)amino)propyl (2S)-2-amino-4-phenylbutanoate [13](0.8 g crude) as pale yellow liquid. The crude was used for the next step without further purification.

### Results:

ELSD analysis: Purity 98.11%, Calculated C₄₉H₉₆N₂O₄Si₂=832.69, Observed=833.50 (m/z, M+H⁺).

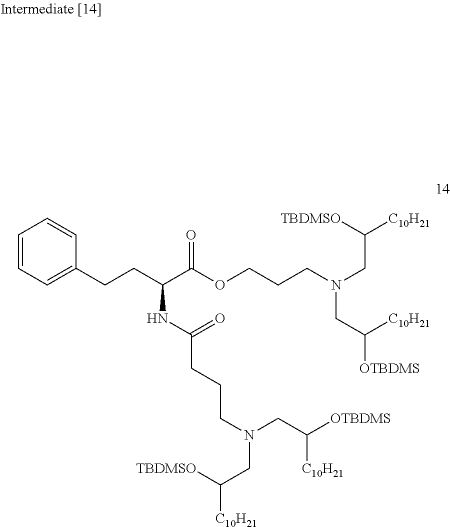

As depicted in Scheme 40: To a stirred solution of 4-(bis{2-[(tert-butyl)bis(methyl)siloxy]dodecyl}amino)butyric acid [6](670 mg, 957 μmol) in dichloromethane (40 mL, 625 mmol), add N,N-dimethyl-4-pyridylamine (58.4 mg, 478 μmol) and EDC·HCl (220 mg, 1.15 mmol) followed by of 3-(bis(2-((tert-butyldimethylsilyl)oxy)dodecyl)amino)propyl (2S)-2-amino-4-phenylbutanoate [13](627 mg, 765 μmol) at room temperature for 16 h. The progress of reaction was monitored by TLC. After completion, reaction mass was diluted with brine solution (20 mL) and extracted with DCM (2×50 mL). The combine organic layers were dried over anhydrous sodium sulphate and concentrated under reduced pressure to get crude. The crude was purified by flash column chromatography (SiO₂: 0-20% EtOAc/Hexane) to get 3-(bis(2-((tert-butyldimethylsilyl)oxy) dodecyl)amino)propyl(13S)-7-(2-((tert-butyldimethylsilyl)oxy)dodecyl)-5-decyl-2,2,3,3-tetramethyl-11-oxo-13-phenethyl-4-oxa-7,12-diaza-3-silatetradecan-14-oate [14] (320 mg 22.06% Yield) as a colourless liquid.

### Results:

ELSD analysis: Purity 99.39%, Calculated C₈₉H₁₇₉N₃O₇Si₄=1514.28, Observed=1514.95 (m/z, M+H⁺).

### Synthesis of Compound XCVI

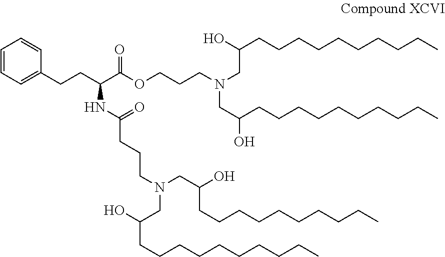 [SMILES: CCCCCCCCCCC(O)CN(CCCOC(=O)[C@H](CCc1ccccc1)NC(=O)CCCN(CC(O)CCCCCCCCCC)CC(O)CCCCCCCCCC)CC(O)CCCCCCCCCC]

As depicted in Scheme 40: To a stirred solution of 3-(bis(2-((tert-butyldimethylsilyl)oxy)dodecyl)amino)propyl (13S)-7-(2-((tert-butyldimethylsilyl)oxy)dodecyl)-5-decyl-2,2,3,3-tetramethyl-11-oxo-13-phenethyl-4-oxa-7,12-diaza-3-silatetradecan-14-oate [14] (310 mg, 205 μmol) in tetrahydrofuran (3 mL, 36.9 mmol) was added hydrogen fluoride pyridine (627 μL, 6.95 mmol) at 0° C., then allowed to stirred at room temperature for 16 h. The progress of reaction was monitored by TLC. After completion, reaction mixture was quenched with cold saturated sodium bicarbonate solution up to pH 8, extraction was done by ethyl acetate (3×10 mL). The organic layers were combine, dried over sodium sulphate, concentrate under reduced pressure to get crude. The crude was purified by flash column chromatography (SiO₂: 0-10% methanol in DCM) to obtain 3-(bis(2-hydroxydodecyl)amino)propyl (2S)-2-(4-(bis(2-hydroxydodecyl)amino)butanamido)-4-phenylbutanoate [Compound XCVI](120 mg, 55.42% Yield) as colourless liquid.

### Results:

¹H-NMR (400 MHz, CDCl3)-δ 7.30-7.24 (m, 2H), 7.23-7.14 (m, 3H), 4.62-4.53 (m, 1H), 4.37-4.20 (m, 1H), 4.14-3.97 (m, 1H), 3.83-3.58 (m, 4H), 2.78-2.23 (m, 16H), 2.20-2.12 (m, 1H), 2.09-1.99 (m, 1H), 1.90-1.74 (m, 4H), 1.50-1.18 (m, 72H), 0.89-0.86 (t, J=7.2 Hz, 12H).

ELSD analysis: Purity 98.80%, Calculated C₆₅H₁₂₃N₃O₇=1057.94, Observed=1058.70 (m/z, M+H⁺).

### Example 41: Synthesis of Compound XCVII

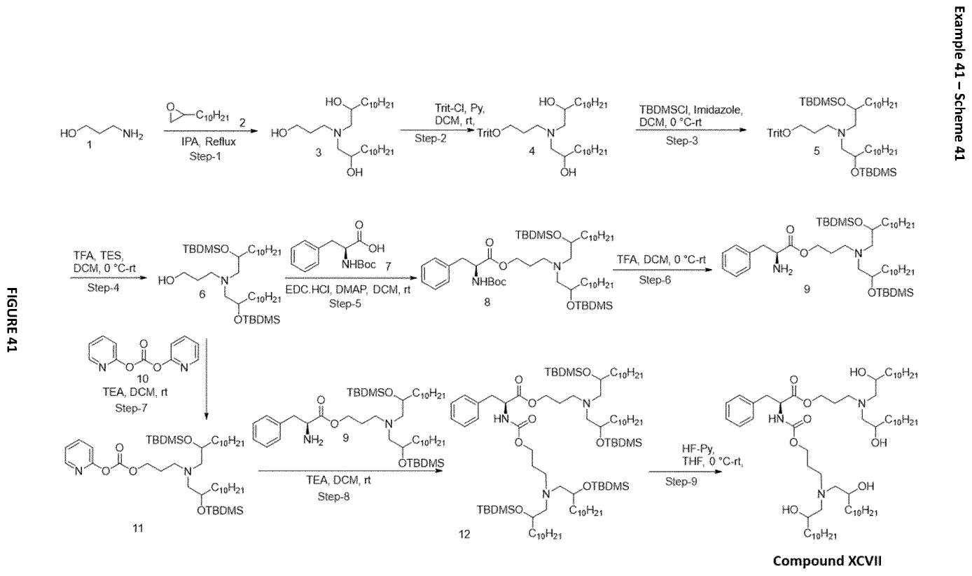

For example, the compounds of the invention may be prepared according to Scheme 41 (as depicted in FIG. 41).

### Intermediate [6]

Synthesis procedure of Intermediate [6] is given in the synthesis for Compound XCV.

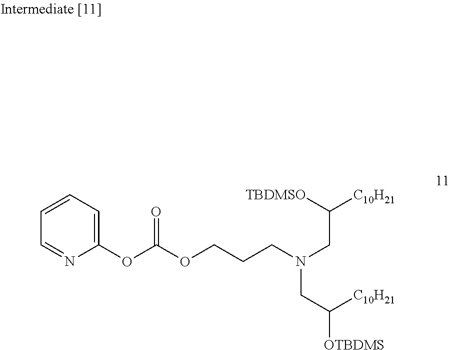

As depicted in Scheme 41: To stirred solution of 3-(bis{2-[(tert-butyl)bis(methyl)siloxy]dodecyl}amino)-1-propanol [6](1.5 g, 2.23 mmol) in dichloromethane (15 mL), add triethylamine (466 μL, 3.35 mmol) followed by the addition of di(pyridin-2-yl) carbonate [10](724 mg, 3.35 mmol), allow to stirred reaction at RT for 16 h. The progress of reaction was monitored by ELSD/TLC. After completion, reaction mass of 3-(bis(2-((tert-butyldimethylsilyl)oxy)dodecyl)amino)propyl pyridin-2-yl carbonate[11] used as it is for next step without further purification.

### Results:

ELSD analysis: Purity 98.69%, Calculated C₄₅H₃₃N₂O₅Si₂=792.62, Observed=793.35 (m/z, M+H⁺).

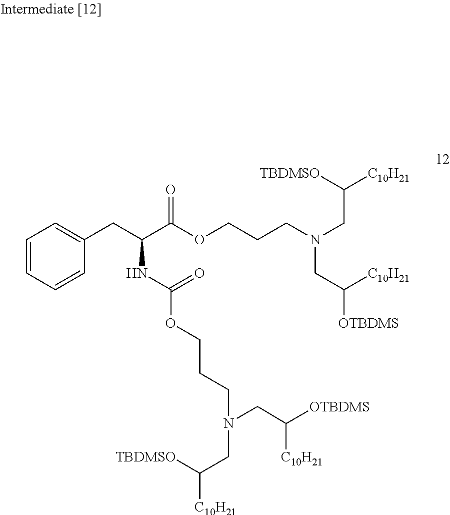

As depicted in Scheme 41: To a stirred solution of 3-(bis(2-((tert-butyldimethylsilyl)oxy)dodecyl)amino)propyl pyridin-2-yl carbonate [11](1.7 g, 2.14 mmol) in dichloromethane (50 mL), were add triethylamine (448 μL, 3.21 mmol) followed by 3-(bis{2-[(tert-butyl)bis(methyl)siloxy]dodecyl}amino)propyl (S)-2-amino-3-phenylpropionate [9](1.76 g, 2.14 mmol) in inert atmosphere, allow to stir at room temperature for 16 h. The progress of reaction was monitored by ELSD/TLC. After completion, reaction mass was evaporated under reduced pressure to get crude. The crude was purified by flash column chromatography (SiO₂: 0-10% EtOAc/Heptane) to get 3-(bis(2-hydroxydodecyl)amino)propyl ((3-(bis(2-hydroxydodecyl)amino) propoxy)carbonyl)-L-phenylalaninate [12](1.4 g, 43.048% Yield) as a pale yellow liquid.

### Results:

ELSD analysis: Purity 99.76%, Calculated C₃₃H₁₇₇N₃O₈Si₄=1516.26, Observed=1517.60 (m/z, M+H⁺).

### Synthesis of Compound XCVII

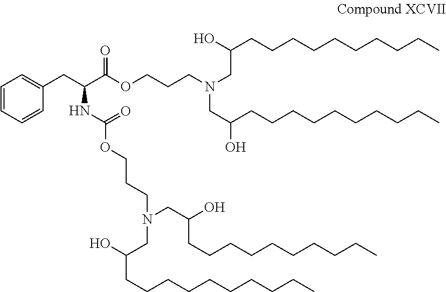 [SMILES: CCCCCCCCCCC(O)CN(CCCOC(=O)N[C@@H](Cc1ccccc1)C(=O)OCCCN(CC(O)CCCCCCCCCC)CC(O)CCCCCCCCCC)CC(O)CCCCCCCCCC]

As depicted in Scheme 41: To stirred solution of 3-(bis{2-[(tert-butyl)bis(methyl)siloxy]dodecyl}amino)propyl (S)-2-{[3-(bis{2-[(tert-butyl)bis(methyl)siloxy]dodecyl}amino)propyl](oxycarbonylamino)}-3-phenylpropionate [12](0.7 g, 461 μmol) in tetrahydrofuran (7 mL, 86 mmol) under nitrogen atmosphere added hydrogen fluoride pyridine (1.6 g, 16.1 mmol) drop wise at 0° C., allow to stirred reaction at RT for 16 h. The progress of reaction was monitored by ELSD/TLC. After completion, reaction mass was diluted with diethyl ether (60 mL) and washed with ice cold water (30 mL), saturated sodium bicarbonate (20 mL), Milli Q water (2×15 ml). The organic layer was collected, dried over sodium sulphate and evaporate under reduced pressure to get 3-(bis(2-hydroxydodecyl)amino)propyl ((3-(bis(2-hydroxydodecyl)amino)propoxy)carbonyl)-L-phenylalaninate [Compound XCVII](0.3 g, 61.32% Yield) as pale yellow liquid.

### Results:

¹H-NMR (400 MHz, CDCl3)-δ 7.32-7.27 (m, 2H), 7.24-7.20 (m, 1H), 7.16-7.13 (m, 2H), 5.36-5.22 (m, 1H), 4.57-4.50 (m, 1H), 4.30-4.07 (m, 4H), 3.67-3.55 (m, 4H), 3.35-2.85 (m, 4H), 2.69-2.47 (m, 6H), 2.45-2.27 (m, 7H), 1.80-1.71 (m, 4H), 1.50-1.21 (m, 74H), 0.89-0.86 (t, J=7.2 Hz, 12H).

ELSD analysis: Purity 99.57%, Calculated C₆₄H₁₂₁N₃O₈=1059.92, Observed=1061.0 (m/z, M+H⁺).

### Example 42: Synthesis of Compound XCVIII

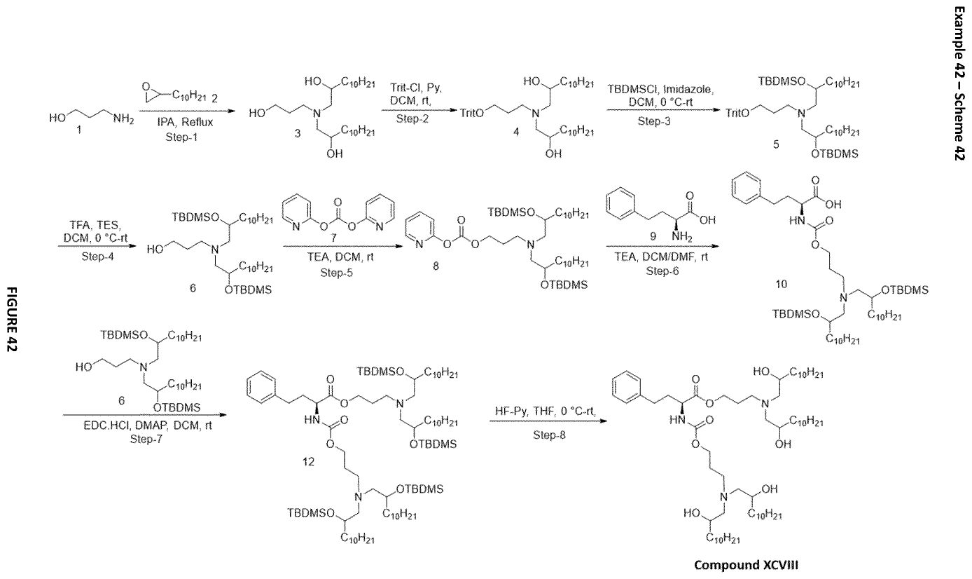

For example, the compounds of the invention may be prepared according to Scheme 42 (as depicted in FIG. 42).

### Intermediate [6]

As depicted in Scheme 42: Synthesis procedure of Intermediate [6] is given in the synthesis for Compound XCV.

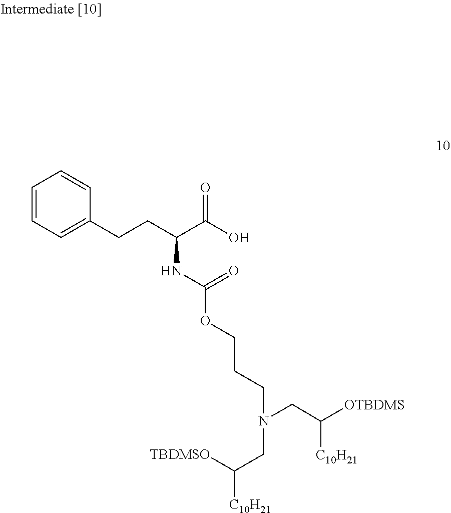

As depicted in Scheme 42: To stirred solution of 3-(bis{2-[(tert-butyl)bis(methyl)siloxy]dodecyl}amino)-1-propanol [6](1 g, 1.49 mmol) in dichloromethane (10 mL, 156 mmol) added triethylamine (1.04 mL, 7.44 mmol) and di(pyridin-2-yl) carbonate [7] (482 mg, 2.23 mmol) and allowed to stir reaction at RT for 16 h to form 3-(bis(2-((tert-butyldimethylsilyl)oxy)dodecyl)amino)propyl pyridin-2-yl carbonate [8]. After this 2-amino-4-phenylbutyric acid (533 mg, 2.97 mmol) was added into reaction mass at RT and allowed to stir for next 16 h. The progress of reaction was monitored by ELSD/TLC. After completion, reaction mass was quenched by water (10 mL) and extracted with DCM (3×15 mL), dried over sodium sulphate, evaporate under reduced pressure to get crude. The crude was purified by flash column chromatography (SiO₂: 0-70% ethyl acetate in hexane) to obtain (14S)-7-(2-((tert-butyldimethylsilyl)oxy)dodecyl)-5-decyl-2,2,3,3-tetramethyl-12-oxo-14-phenethyl-4,11-dioxa-7,13-diaza-3-silapentadecan-15-oic acid [10](0.4 g, 30.65% Yield) as pale yellow liquid.

### Results:

ELSD analysis: Purity 99.60%, Calculated C₅₀H₉₆N₂O₆Si₂=876.68, Observed=877.40 (m/z, M+H⁺).

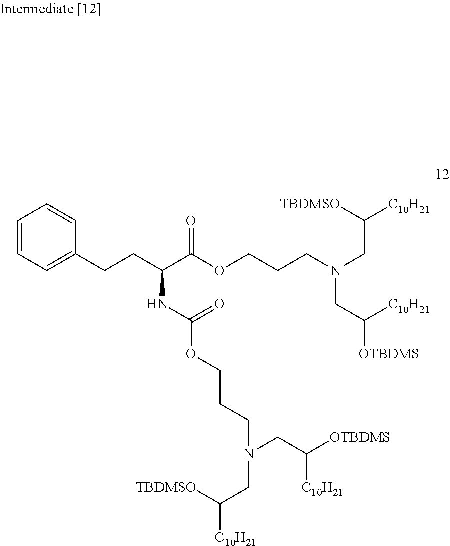

As depicted in Scheme 42: To a stirred solution of (S)-2-[3-(bis{2-[(tert-butyl)bis(methyl)siloxy]dodecyl}amino)propoxycarbonylamino]-4-phenylbutyric acid [10](0.4 g, 456 μmol) in dichloromethane (10 mL, 156 mmol), add N,N-dimethyl-4-pyridylamine (27.8 mg, 0.5 eq., 228 μmol) followed by the addition of 2-methyl-2,6,8-triaza-6,7-decadiene-hydrogen chloride (1/1) (131 mg, 1.5 eq., 684 μmol) at room temperature. After this add 3-(bis{2-[(tert-butyl)bis(methyl)siloxy]dodecyl}amino)-1-propanol [6](306 mg, 456 μmol). Allow the reaction mixture to stir at room temperature for 16 h. Progress of reaction was monitored by ELSD/TLC, after completion the reaction (sm was consumed), reaction mixture was quenched with brine solution (5 ml) and extracted with DCM (2×15 ml). The combined organic layer was dried over anhydrous sodium sulphate, concentrated under reduced pressure to get crude and crude was purified by column chromatography (0-50% Ethyl acetate/Heptane) to get desired product 3-(bis{2-[(tert-butyl)bis(methyl)siloxy]dodecyl}amino)propyl (S)-2-[3-(bis{2-[(tert-butyl)bis(methyl)siloxy]dodecyl}amino)propoxycarbonylamino]-4-phenylbutyrate [12](0.293 g 41.96% Yield) as pale yellow liquid.

### Result:

ELSD analysis: Purity 99.88%, Calculated C₈₉H₁₇₉N₃O₈Si₄=1530.28, Observed=1531.75 (m/z, M+H⁺).

### Synthesis of Compound XCVIII

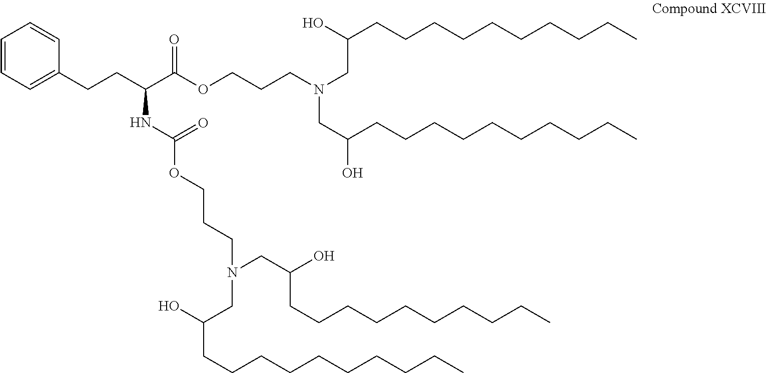 [SMILES: CCCCCCCCCCC(O)CN(CCCOC(=O)N[C@@H](CCc1ccccc1)C(=O)OCCCN(CC(O)CCCCCCCCCC)CC(O)CCCCCCCCCC)CC(O)CCCCCCCCCC]

As depicted in Scheme 42: To a stirred solution of 3-(bis{2-[(tert-butyl)bis(methyl)siloxy]dodecyl}amino)propyl (S)-2-{[3-(bis{2-[(tert-butyl)bis(methyl)siloxy]dodecyl}amino)propyl](oxycarbonylamino)}-4-phenylbutyrate [12](290 mg, 189 μmol) in tetrahydrofuran (3 mL, 36.9 mmol) was added hydrogen fluoride pyridine (597 μL, 6.63 mmol) at 0° C., allowed to stir at room temperature for 16 h. The progress of reaction was monitored by TLC/ELSD. After completion, reaction mixture was quenched with cold saturated sodium bicarbonate solution up to pH 8, extraction was done by ethyl acetate (3×10 mL). The organic layers were combine, dried over sodium sulphate, concentrate under reduced pressure to get crude. The crude was purified by flash column chromatography (SiO₂: 0-10% methanol in DCM) to obtain 3-(bis(2-hydroxydodecyl)amino)propyl (2S)-2-(((3-(bis(2-hydroxydodecyl)amino)propoxy) carbonyl) amino)-4-phenylbutanoate [Compound XCVIII](130 mg, 63.89% Yield) as colourless liquid.

### Result:

¹H-NMR (400 MHz, CDCl3)-δ 7.30-7.28 (m, 2H), 7.23-7.16 (m, 3H), 5.90-5.30 (br, 1H), 4.40-3.98 (m, 5H), 3.82-3.60 (m, 4H), 2.97-2.38 (m, 16H), 2.21-2.12 (m, 1H), 2.10-1.96 (m, 1H), 1.48-1.37 (m, 10H), 1.31-1.22 (m, 68H), 0.87-0.83 (t, J=7.2 Hz, 12H).

ELSD analysis: Purity 99.75%, Calculated C₆₅H₁₂₃N₃O₈=1073.93, Observed=1074.50 (m/z, M+H+).

### Example 43: Synthesis of Compound XCIX

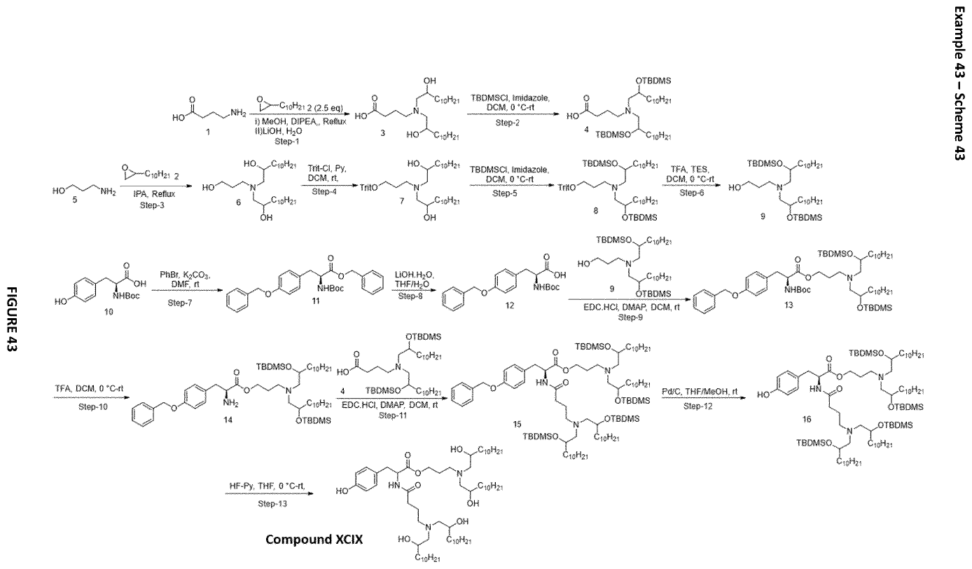

For example, the compounds of the invention may be prepared according to Scheme 43 (as depicted in FIG. 43).

### Intermediate [4] and Intermediate [9]

As depicted in Scheme 43: Synthesis procedure of Intermediate [4] and Intermediate [9] is given in the synthesis for Compound XCV.

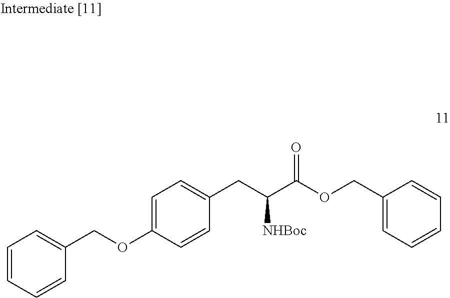 [SMILES: CC(C)(C)OC(=O)N[C@@H](Cc1ccc(OCc2ccccc2)cc1)C(=O)OCc1ccccc1]

As depicted in Scheme 43: To a stirred solution of (S)-2-[(tert-butyl)(oxycarbonylamino)]-3-(p-hydroxyphenyl)propionic acid [10](2.5 g, 8.89 mmol) in dimethylformamide (0.1 L) were added dipotassium carbonate (6.14 g, 44.4 mmol) and (bromomethyl)benzene (4.56 g, 26.7 mmol). The reaction mixture was stirred at RT for 32 h. The progress of the reaction was monitored by ELSD/TLC. After completion of the reaction, the reaction mass was quenched with an ice-cold solution of brine (15 mL) and extracted with ethyl acetate (3×30 mL). The organic layer was collected, dried over sodium sulphate and concentrated under reduced pressure to get crude. The crude was purified by flash column chromatography (SiO₂: 0-20% ethyl acetate in hexane) to give benzyl (S)-3-[p-(benzyloxy)phenyl]-2-[(tert-butyl)(oxycarbonylamino)]propionate [11](3 g, 73.14% Yield) as a colourless solid.

### Results:

¹H NMR (400 MHz, CDCl₃): δ 7.44-7.29 (m, 10H), 6.99 (d, J=8.4 Hz, 2H), 6.84 (d, J=8.4 Hz, 2H), 5.29-4.96 (m, 5H), 4.61-4.60 (m, 1H), 3.02 (d, J=5.6 Hz, 2H), 1.42 (s, 9H).

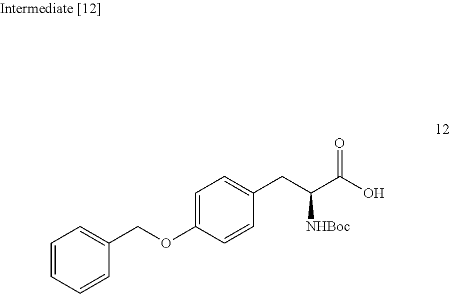 [SMILES: CC(C)(C)OC(=O)N[C@@H](Cc1ccc(OCc2ccccc2)cc1)C(=O)O]

As depicted in Scheme 43: To a stirred solution of benzyl (S)-3-[p-(benzyloxy)phenyl]-2-[(tert-butyl) (oxycarbonylamino)]propionate [11](3 g, 6.5 mmol) in methanol (23 mL), water (23 mL), and tetrahydrofuran (7.89 mL) was added lithium hydroxide monohydrate (1.09 g, 26 mmol). The reaction mixture was stirred at RT for 32 h. The progress of the reaction was monitored by ELSD/TLC. After completion of reaction, the reaction mass was quenched with a saturated solution of citric acid (pH 6) and extracted with DCM (3×50 mL). The organic layer was collected, dried over sodium sulphate and concentrated under reduced pressure to get crude. The crude was purified by flash column chromatography (SiO₂: 0-70% ethyl acetate in hexane) to get (S)-3-[p-(benzyloxy)phenyl]-2-[(tert-butyl)(oxycarbonylamino)]propionic acid [12](1.8 g, 74.55% Yield) as a pale yellow liquid.

### Results:

ELSD analysis: Purity 99.36%, Calculated C₂₁H₂₅NO₅=371.17, Observed=394.15 (m/z, M+Na⁺).

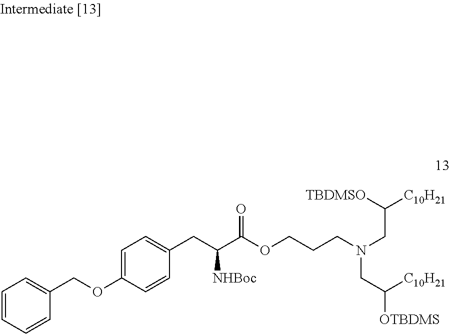

As depicted in Scheme 43: To a stirred solution of (S)-3-[p-(benzyloxy)phenyl]-2-(tert-butoxycarbonylamino)propionic acid [12](0.5 g, 1.35 mmol) in dichloromethane (20 mL), were added N,N-dimethyl-4-pyridylamine (82.2 mg, 673 μmol), EDC·HCl (387 mg, 2.02 mmol). The reaction mixture was stirred at RT for 30 min and added 3-(bis{2-[(tert-butyl)bis(methyl)siloxy]dodecyl}amino)-1-propanol [9](905 mg, 1.35 mmol). The reaction mixture was stirred at RT for 48 h. The progress of reaction was monitored by ELSD/TLC. After completion of reaction, reaction mass quenched with water (30 mL) and extracted with DCM (3×100 mL). The organic layer was combined and dried over sodium sulphate and concentrated under reduced pressure to get crude. The crude was purified by flash column chromatography (SiO₂: 0-10% ethyl acetate in hexane) to get 3-(bis{2-[(tert-butyl)bis(methyl)siloxy]dodecyl}amino)propyl (S)-3-[p-(benzyloxy)phenyl]-2-(tert-butoxycarbonylamino)propionate [13](950 mg, 68.80% Yield) as a pale yellow liquid.

### Results:

ELSD analysis: Purity 97.66%, Calculated C₆₀H₁₀₈N₂O₇Si₂=1024.77, Observed=1025.50 (m/z, M+H⁺).

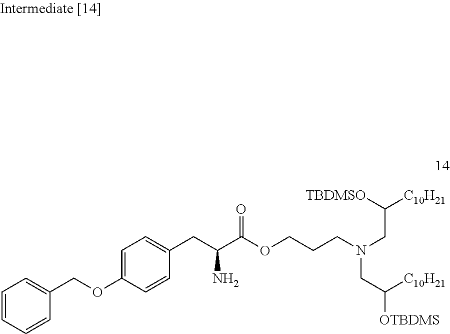

As depicted in Scheme 43: To a stirred solution of 3-(bis{2-[(tert-butyl)bis(methyl)siloxy]dodecyl}amino)propyl (S)-3-[p-(benzyloxy)phenyl]-2-(tert-butoxycarbonylamino)propionate [13](1.5 g, 1.46 mmol) in dichloromethane (15 mL) was slowly added trifluoroacetic acid (2.53 mL, 33 mmol) at 0° C. The progress of reaction was monitored by ELSD/TLC. After completion of reaction, reaction mass was quenched with saturated solution of sodium bicarbonate (make pH 8-9) and extracted with DCM (2×20 mL).

The combined organic layer was dried over anhydrous sodium sulphate and concentrated under reduced pressure to get 3-(bis(2-((tert-butyldimethylsilyl)oxy)dodecyl)amino)propyl (2S)-2-amino-3-(4-(benzyloxy)phenyl) propanoate [14](1.33 g, 98.25% Yield) as apale yellow liquid.

### Results:

ELSD analysis: Purity 97.37%, Calculated C₅₅H₁₀₀N₂O₅Si₂=924.72, Observed=925.50 (m/z, M+H⁺).

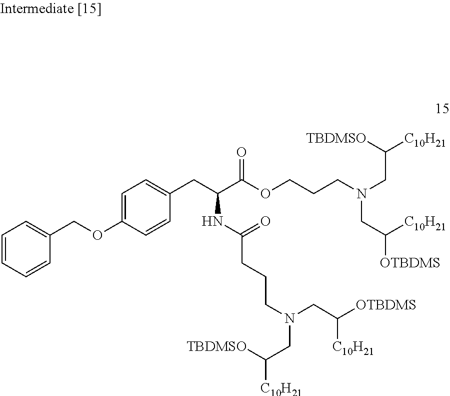

As depicted in Scheme 43: To a stirred solution of 4-(bis{2-[(tert-butyl)bis(methyl)siloxy]dodecyl}amino)butyric acid [4](1.1 g, 1.57 mmol) in dichloromethane (30 mL), were added N,N-dimethyl-4-pyridylamine (96 mg, 785 μmol) and EDC·HCl (452 mg, 2.36 mmol) followed by the addation 3-(bis(2-((tert-butyldimethylsilyl)oxy)dodecyl)amino)propyl (2S)-2-amino-3-(4-(benzyloxy)phenyl) propanoate [14](1.45 g, 1.57 mmol) at room temperature. The reaction mixture was stirred at room temperature for 16 h. The progress of reaction was monitored by ELSD/TLC. After completion of reaction, reaction mass was quenched with brine solution (15 mL) and extracted with DCM (2×50 mL). The combined organic layer was dried over anhydrous sodium sulphate and concentrated under reduced pressure to get crude. The crude was purified by flash column chromatography (SiO₂: 0-15% EtOAc/Heptane) to get desired product 3-(bis(2-((tert-butyldimethylsilyl)oxy)dodecyl)amino)propyl (13S)-13-(4-(benzyloxy)benzyl)-7-(2-((tert-butyldimethylsilyl)oxy)dodecyl)-5-decyl-2,2,3,3-tetramethyl-11-oxo-4-oxa-7,12-diaza-3-silatetradecan-14-oate [15](1.3 g, 51.47% Yield) as a pale yellow liquid.

### Results:

ELSD analysis: Purity 99.37%, Calculated C₉₅H₁₃₃N₃OSi₄=1606.31, Observed=1607.85 (m/z, M+H⁺).

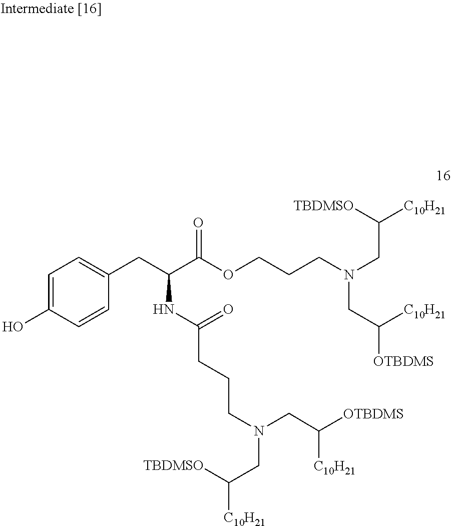

As depicted in Scheme 43: To a stirred solution of 3-(bis(2-((tert-butyldimethylsilyl)oxy)dodecyl)amino)propyl (13S)-13-(4-(benzyloxy)benzyl)-7-(2-((tert-butyldimethylsilyl)oxy)dodecyl)-5-decyl-2,2,3,3-tetramethyl-11-oxo-4-oxa-7,12-diaza-3-silatetradecan-14-oate [15](1.3 g, 809 μmol) in methanol (27.9 mL) was added Palladium on carbon 10%, wet 50% water (654 mg, 614 μmol). The reaction mixture was degassed and stirred at room temperature overnight under hydrogen atmosphere (balloon pressure). The progress of reaction was monitored by TLC. The reaction mixture was filtered through celite and washed with MeOH (100 mL). The filtrate was concentrated under reduced pressure to obtain 3-(bis(2-((tert-butyldimethylsilyl) oxy)dodecyl) amino)propyl (4-(bis(2-((tert-butyldimethylsilyl)oxy)dodecyl)amino)butanoyl)-L-tyrosinate [16](950 mg, 77.41% Yield) as a colourless liquid.

### Results:

ELSD analysis: Purity 99.72%, Calculated C₃₃H₁₇₇N₃OSi₄=1516.26, Observed=1517.75 (m/z, M+H+).

### Synthesis of Compound XCIX

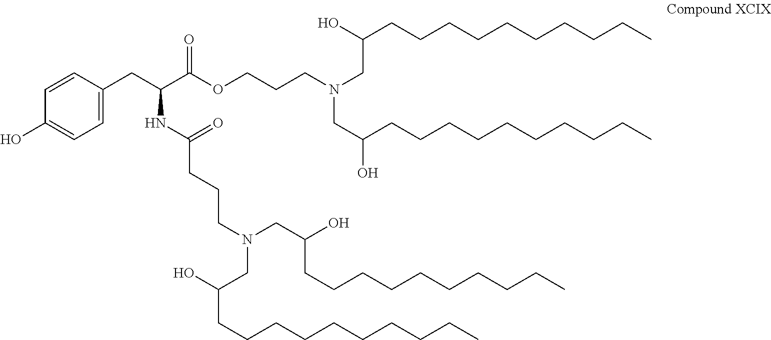 [SMILES: CCCCCCCCCCC(O)CN(CCCOC(=O)[C@H](Cc1ccc(O)cc1)NC(=O)CCCN(CC(O)CCCCCCCCCC)CC(O)CCCCCCCCCC)CC(O)CCCCCCCCCC]

As depicted in Scheme 43: To a stirred solution of 3-(bis(2-((tert-butyldimethylsilyl)oxy)dodecyl)amino)propyl (4-(bis(2-((tert-butyldimethylsilyl)oxy)dodecyl)amino)butanoyl)-L-tyrosinate [16](0.7 g, 461 μmol) in tetrahydrofuran (7 mL) was added hydrogen fluoride pyridine (1.6 g, 16.1 mmol) drop wise at cooling condition under nitrogen atmosphere. The reaction mixture was stirred at RT for 16 h. The progress of reaction was monitored by ELSD/TLC. After completion of reaction, reaction mass was diluted with diethyl ether (10 mL) and washed with ice cold water (5 mL), saturated sodium bicarbonate (8 mL), Milli Q water (2×8 mL). The organic layer was collected, dried over sodium sulphate and concentrated under reduced pressure to get 3-(bis(2-hydroxydodecyl)amino)propyl (4-(bis(2-hydroxydodecyl)amino)butanoyl)-L-tyrosinate [Compound XCIX](450 mg, 91.98% Yield) as a pale yellow liquid.

### Results:

¹H-NMR (400 MHz, CDCl₃): δ 6.99 (d, J=8.4 Hz, 2H), 6.79-6.74 (m, 2H), 6.62-6.30 (m, 1H), 4.82-4.70 (m, 1H), 4.22-4.02 (m, 2H), 3.70-3.5 (brm, 4H), 3.03-2.92 (m, 2H), 2.57-2.23 (17H), 1.72-1.66 (4H), 1.50-1.20 (m, 74H), 0.88 (t, J=6.4 Hz, 12H).

ELSD analysis: Purity 98.65%, Calculated C₆₄H₁₂₁N₃O₃=1059.92, Observed=1061.05 (m/z, M+H⁺).

### Example 44: Synthesis of Compound C

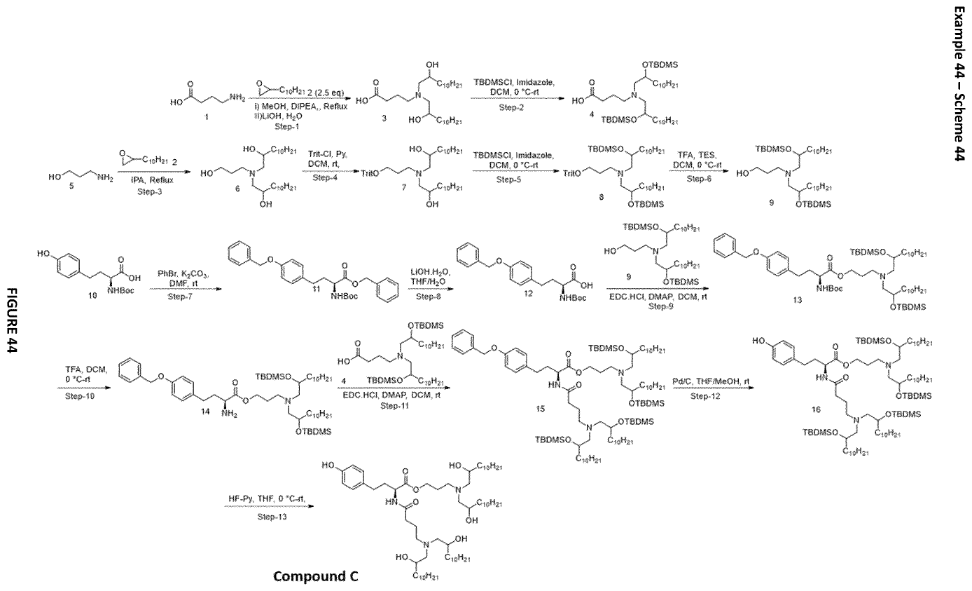

For example, the compounds of the invention may be prepared according to Scheme 44 (as depicted in FIG. 44).

### Intermediate [4] and Intermediate [9]

As depicted in Scheme 44: Synthesis procedure of Intermediate [4] and Intermediate [9] is given in the synthesis for Compound XCV.

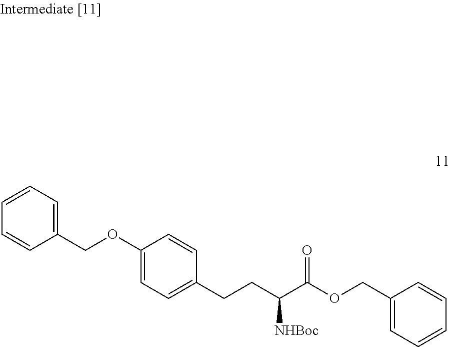 [SMILES: CC(C)(C)OC(=O)N[C@@H](CCc1ccc(OCc2ccccc2)cc1)C(=O)OCc1ccccc1]

As depicted in Scheme 44: To a stirred suspension of (S)-2-[(tert-butyl)(oxycarbonylamino)]-4-(p-hydroxyphenyl)butyric acid [10](0.5 g, 1.69 mmol) and dipotassium carbonate (1.17 g, 8.46 mmol) in dimethylformamide (10 mL), was added (bromomethyl)benzene (603 μL, 5.08 mmol) at ° C., under inert atmosphere. The reaction mixture was stirred at RT for 32 h. The progress of reaction was monitored by TLC. After completion of reaction, reaction mass was quenched with ice cold brine solution (15 mL) and extracted with ethyl acetate (3×30 mL). The organic layer was collected, dried over anhy. sodium sulphate and concentrated under reduced pressure to get crude. The crude was purified by flash column chromatography (0-20% ethyl acetate in hexane) to give benzyl (S)-4-[p-(benzyloxy)phenyl]-2-[(tert-butyl)(oxycarbonylamino)]butyrate [11](0.6 g, 74.52% Yield) as a pale yellow liquid.

### Results:

¹H NMR (400 MHz, CDCl₃): δ 7.45-7.31 (m, 11H), 7.05 (d, J=8.4 Hz, 2H), 6.90 (d, J=8.4 Hz, 2H), 5.31-5.11 (m, 2H), 5.08-5.04 (m, 2H), 4.41-4.39 (m, 1H), 2.65-2.51 (m, 2H), 2.11-2.09 (m, 1H), 1.95-1.87 (m, 1H), 1.46 (s, 9H).

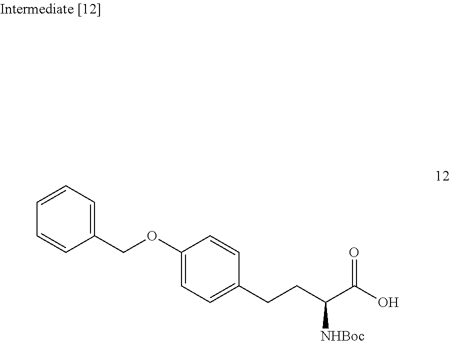 [SMILES: CC(C)(C)OC(=O)N[C@@H](CCc1ccc(OCc2ccccc2)cc1)C(=O)O]

As depicted in Scheme 44: To a stirred solution of benzyl (S)-4-[p-(benzyloxy)phenyl]-2-[(tert-butyl) (oxycarbonylamino)]butyrate (0.6 g, 1.26 mmol) in methanol (5 mL, 123 mmol), tetrahydrofuran (5 mL, 61.4 mmol) and water (5 mL, 278 mmol) was added lithium hydroxide (121 mg, 4 eq., 5.05 mmol). The reaction mixture was stirred at RT for 16 h. The progress of reaction was monitored by TLC. After completion of reaction, reaction mass was quenched saturated solution of citric acid (PH upto 5) and extracted with DCM (2×50 mL). The organic layer was collected dried over sodium sulphate and evaporate under reduced pressure to get crude. The crude was purified over silica using 0-70% ethyl acetate in hexane to get desired product as (S)-4-[p-(benzyloxy)phenyl]-2-[(tert-butyl)(oxycarbonylamino)]butyric acid (475 mg, 97.68% Yield) as a colourless liquid.

### Results:

ELSD analysis: Purity 94.13%, Calculated C₂₂H₂₇NO₅=385.19, Observed=403.29 (m/z, M+NH₄⁺).

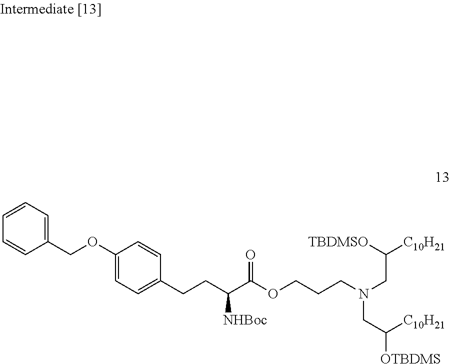

As depicted in Scheme 44: To a stirred solution of (S)-4-[p-(benzyloxy)phenyl]-2-[(tert-butyl)(oxycarbonylamino)]butyric acid [12](475 mg, 1.23 mmol) in dichloromethane (100 mL), were added N,N-dimethyl-4-pyridylamine (75.3 mg, 616 μmol) and EDC·HCl (354 mg, 1.85 mmol) at room temperature. The reaction mixture was stirred for 30 min at RT and added 3-(bis{2-[(tert-butyl) bis(methyl)siloxy]dodecyl}amino)-1-propanol [9] (828 mg, 1.23 mmol). The reaction mixture was stirred at RT for 16 h. The progress of reaction was monitored by TLC. After completion of reaction, reaction mixture was diluted with DCM (50 mL) washed with water (2×20 mL) and brine solution (10 mL). The organic layer was collect, dried over sodium sulphate, concentrated under reduced pressure to get crude. The crude was purified by flash column chromatography (SiO₂: 0-40% ethyl acetate in heptane) to afford 3-(bis{2-[(tert-butyl)bis(methyl)siloxy]dodecyl}amino)propyl (S)-4-[p-(benzyloxy)phenyl]-2-[(tert-butyl) (oxycarbonylamino)]butyrate [13](630 mg, 49.17% Yield) as a pale yellow.

### Results:

ELSD analysis: Purity 99.60%, Calculated Ce₁H₁₁₀N₂O₇Si₂=1038.79, Observed=1039.40 (m/z, M+H⁺).

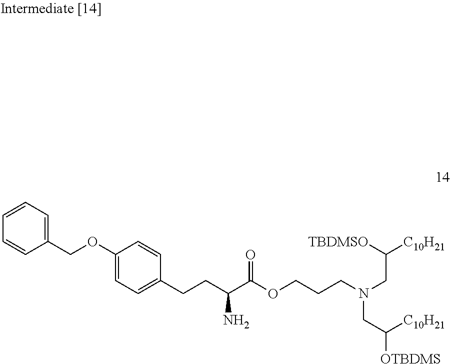

As depicted in Scheme 44: To a stirred solution of 3-(bis{2-[(tert-butyl)bis(methyl)siloxy]dodecyl}amino)propyl (S)-4-[p-(benzyloxy)phenyl]-2-[(tert-butyl)(oxycarbonylamino)]butyrate [13](630 mg, 606 μmol) in dichloromethane (10 mL), was added trifluoroacetic acid (696 μL, 9.09 mmol) dropwise at 0° C. The reaction mixture was stirred at RT for 4 h. The progress of reaction was monitored by TLC. After completion of reaction, reaction mass was quenched by with saturated solution of sodium bicarbonate (make pH 8-9) and extracted with DCM (2×25 mL). The combined organic layer was collected, dried over anhydrous sodium sulphate and concentrated under reduced pressure to get of 3-(bis{2-[(tert-butyl)bis(methyl)siloxy]dodecyl}amino)propyl (S)-2-amino-4-[p-(benzyloxy)phenyl]butyrate [14](540 mg, 94.84% Yield) as a pale yellow liquid which was used in the next step without further purification.

### Results:

ELSD analysis: Purity 99.15%, Calculated C₅₆H₁₀₂N₂O₅Si₂=938.73, Observed=939.40 (m/z, M+H⁺).

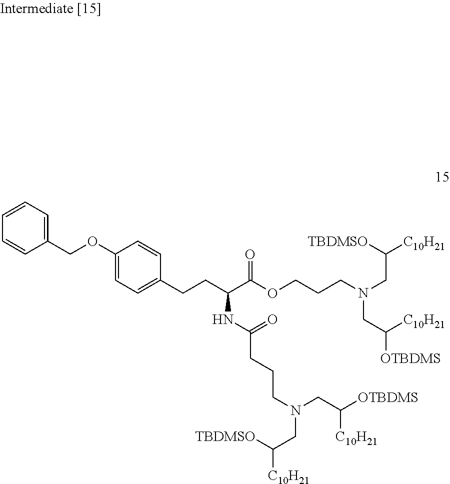

As depicted in Scheme 44: To a stirred solution 4-(bis{2-[(tert-butyl)bis(methyl)siloxy]dodecyl}amino)butyric acid [4](402 mg, 575 μmol) in dichloromethane (10 mL), were added N,N-dimethyl-4-pyridylamine (70.2 mg, 575 μmol) and EDC·HCl (165 mg, 862 μmol) followed by 3-(bis{2-[(tert-butyl)bis(methyl)siloxy]dodecyl}amino)propyl (S)-2-amino-4-[p-(benzyloxy)phenyl]butyrate [14] (540 mg, 575 μmol) at room temperature. The reaction mixture was stirred at RT for 16 h. The progress of reaction was monitored by TLC. After completion of reaction, reaction mass was diluted with saturated brine solution (2×10 mL), and extracted with DCM (2×50 mL). The combined organic layer was dried over anhydrous sodium sulphate and concentrated under reduced pressure to get crude. The crude was then purified by flash column chromatography (Silica 0-20% EtOAc/Heptane) to obtain 3-(bis{2-[(tert-butyl)bis(methyl)siloxy]dodecyl}amino)propyl (S)-4-[p-(benzyloxy)phenyl]-2-[4-(bis{2-[(tert-butyl)bis(methyl) siloxy]dodecyl}amino)butyrylamino]butyrate [15](560 mg, 60.8% Yield) as a pale yellow liquid.

### Results:

ELSD analysis: Purity 99.29%, Calculated C₉₆H₁₈₅N₃O₈Si₄=1620.32, Observed=1621.75 (m/z, M+H⁺).

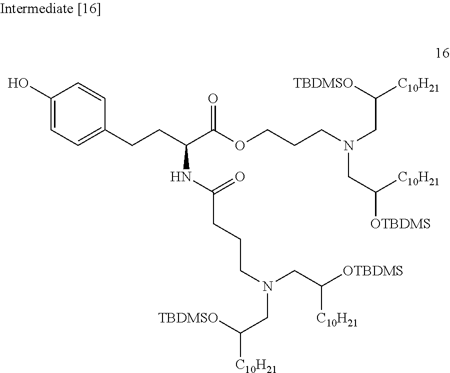

As depicted in Scheme 44: To a stirred solution of 3-(bis{2-[(tert-butyl)bis(methyl)siloxy]dodecyl}amino)propyl (S)-4-[p-(benzyloxy)phenyl]-2-[4-(bis{2-[(tert-butyl)bis(methyl)siloxy]dodecyl}amino) butyrylamino]butyrate [15](540 mg, 333 μmol) in tetrahydrofuran (10 mL, 123 mmol), was added Palladium on carbon 10%, wet 50% water (549 mg, 516 μmol). The reaction mixture was degassed and stirred at room temperature overnight under hydrogen atmosphere (balloon pressure). The progress of reaction was monitored by TLC. The reaction mixture was filtered through celite and washed with MeOH (100 mL). The filtrate was concentrated under reduced pressure to obtain 3-(bis{2-[(tert-butyl)bis(methyl)siloxy]dodecyl}amino)propyl (S)-2-[4-(bis{2-[(tert-butyl)bis(methyl)siloxy]dodecyl}amino)butyrylamino]-4-(p-hydroxyphenyl) butyrate [16](350 mg, 68.63% Yield) as a colourless liquid.

### Results:

ELSD analysis: Purity 99.05%, Calculated C₈₉H₁₇₉N₃O₈Si₄=1530.28, Observed=1531.70 (m/z, M+H⁺).

### Synthesis of Compound C

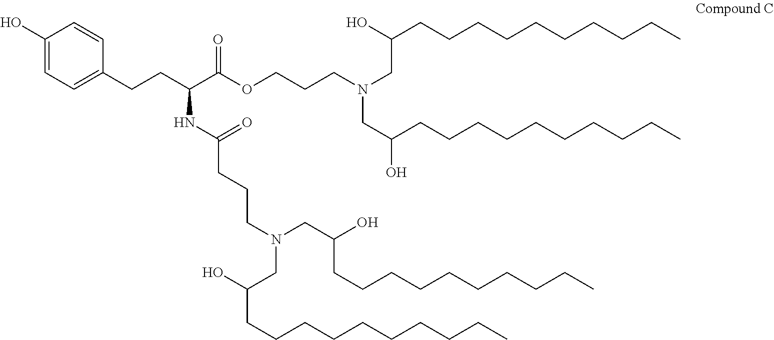 [SMILES: CCCCCCCCCCC(O)CN(CCCOC(=O)[C@H](CCc1ccc(O)cc1)NC(=O)CCCN(CC(O)CCCCCCCCCC)CC(O)CCCCCCCCCC)CC(O)CCCCCCCCCC]

As depicted in Scheme 44: To a stirred solution of 3-(bis{2-[(tert-butyl)bis(methyl)siloxy]dodecyl}amino)propyl (S)-2-[4-(bis{2-[(tert-butyl)bis(methyl)siloxy]dodecyl}amino)butyrylamino]-4-(p-hydroxyphenyl) butyrate [16](320 mg, 209 μmol) in tetrahydrofuran (4 mL) was added Hydrogen fluoride pyridine (640 μL, 7.1 mmol) drop wise at 0° C. under nitrogen atmosphere. The reaction mixture was stirred at RT for 16 h. The progress of reaction was monitored by ELSD/TLC. After completion of reaction, reaction mass was diluted with diethyl ether (30 mL), washed with ice cold water (10 mL), saturated sodium bicarbonate (10 mL) and Milli Q water (2×15 mL). The organic layer was collected, dried over sodium sulphate and concentrated under reduced pressure to get 3-[bis(2-hydroxydodecyl)amino]propyl (S)-2-{4-[bis(2-hydroxydodecyl)amino]butyrylamino}-4-(p-hydroxyphenyl)butyrate [Compound C](185 mg, 82.39% Yield) as a pale yellow liquid.

### Results:

¹H-NMR (400 MHz, CDCl₃): δ 7.01-6.99 (d, J=8.4 Hz, 2H), 6.78-6.75 (m, 2H), 6.07-5.95 (m, 1H), 4.62-4.50 (m, 1H), 4.31-4.00 (m, 2H), 3.71-3.55 (brs, 4H), 2.72-2.30 (m, 7H), 2.50-2.25 (m, 7H), 2.20-1.90 (m, 5H), 1.82-1.72 (m, 2H), 1.71-1.62 (m, 2H), 1.50-1.20 (m, 72H), 0.88 (t, J=6.4 Hz, 12H).

ELSD analysis: Purity 99.66%, Calculated C₆₅H₁₂₃N₃O₈=1073.93, Observed=1074.85 (m/z, M+H⁺).

### Example 45: Synthesis of Compound CI

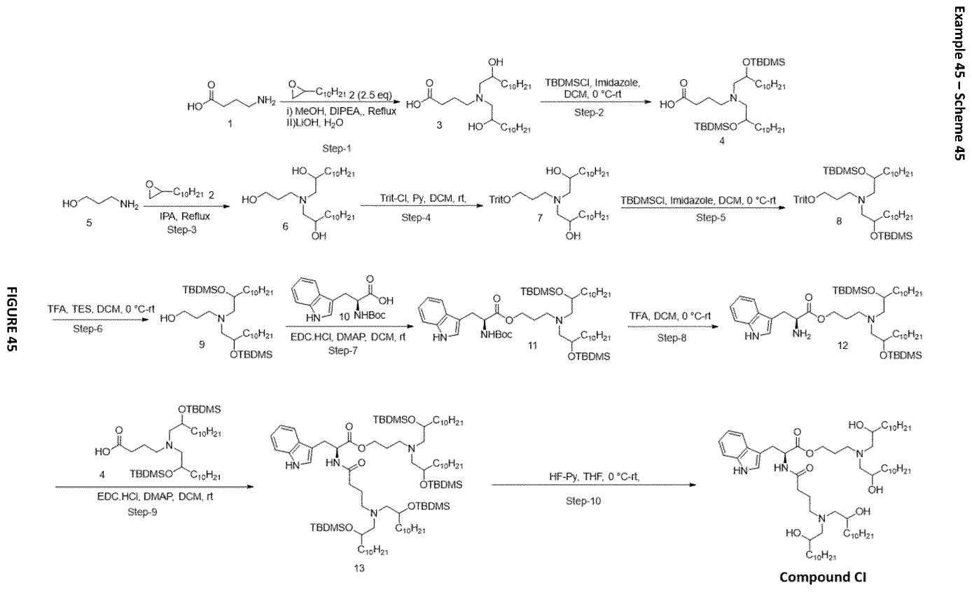

For example, the compounds of the invention may be prepared according to Scheme 45 (as depicted in FIG. 45).

### Intermediate [4] and Intermediate [9]

As depicted in Scheme 45: Synthesis procedure of Intermediate [4] and Intermediate [4] is given in the synthesis for Compound XCV.

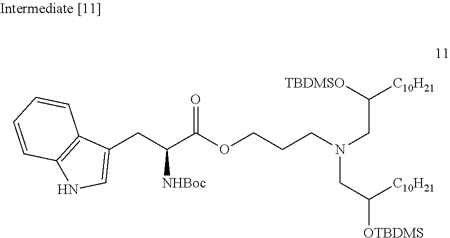

As depicted in Scheme 45: To a stirred solution of 2-(tert-butoxycarbonylamino)-3-(3-indolyl)propionic acid [10](1 g, 3.29 mmol) in dichloromethane (20 mL), were add N,N-dimethyl-4-pyridylamine (201 mg, 1.64 mmol) and EDC·HCl (945 mg, 4.93 mmol). The reaction mixture was stirred for 30 min at RT and added 3-(bis{2-[(tert-butyl)bis(methyl)siloxy]dodecyl}amino)-1-propanol [9](1.99 g, 2.96 mmol). The reaction mixture was stirred at room temperature for 16 h. The progress of reaction was monitored by TLC. After completion of reaction, reaction mass was diluted with water (20 mL) and extracted with DCM (2×50 mL). The combined organic layer was dried over anhydrous sodium sulphate and concentrated under reduced pressure to get crude. The crude was then purified over silica using (0-30% EtOAc/Hexane) to get 3-(bis(2-((tert-butyldimethylsilyl)oxy)dodecyl)amino)propyl (tert-butoxycarbonyl)-L-tryptophanate [11](1.6 g, 50.8% Yield) as a colourless semi solid.

### Result:

ELSD analysis: Purity 98.56%, Calculated C₅₅H₁₀₃N₃O₆Si₂=957.74, Observed=958.50 (m/z, M+H⁺).

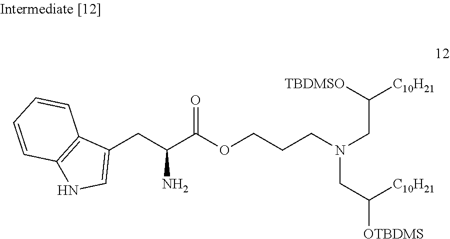

As depicted in Scheme 45: To a stirred solution of 3-(bis(2-((tert-butyldimethylsilyl)oxy)dodecyl)amino)propyl (tert-butoxycarbonyl)-L-tryptophanate [11](1.6 g, 1.67 mmol) in dichloromethane (10 mL) was added trifluoroacetic acid (1.28 mL, 16.7 mmol) drop by drop at 0° C. The reaction mixture was stirred at room temperature a for 3 h. The progress of reaction was monitored by TLC. After completion of reaction, reaction mass was concentrated under vacuum. To the crude diethyl ether was added and concentrated under vacuum remove excess amount of TFA. The crude was dissolved in DCM and added triethyl amine (pH 8-9) after that water was added. The organic layer was separated, dried over anhydrous sodium sulphate and concentrated under reduced pressure to get 3-(bis{2-[(tert-butyl)bis(methyl)siloxy]dodecyl}amino)propyl 2-amino-3-(3-indolyl)propionate (1.4 g crude) as a pale yellow oil, which was used in next step without further purification.

### Result:

ELSD analysis: Purity 97.06%, Calculated C₅₀H₉₅N₃O₄Si₂=857.69, Observed=858.50 (m/z, M+H⁺).

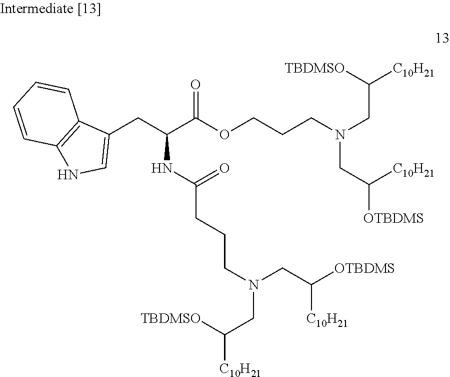

As depicted in Scheme 45: To a stirred solution of 4-(bis{2-[(tert-butyl)bis(methyl)siloxy]dodecyl}amino)butyric acid [4](1.17 g, 1.67 mmol) in dichloromethane (25 mL) were added N,N-dimethyl-4-pyridylamine (102 mg, 833 μmol) and EDC·HCl (479 mg, 2.5 mmol) followed by 3-(bis(2-((tert-butyldimethylsilyl)oxy)dodecyl)amino)propyl L-tryptophanate [12](1.43 g, 1.67 mmol). The reaction mixture was stirred for 16 h at room temperature. The progress of reaction was monitored by TLC. After completion of reaction, reaction mass was diluted with brine solution (15 mL) and extracted with DCM (2×50 mL). The combined organic layer was dried over anhydrous sodium sulphate and concentrated under reduced pressure to get crude. The crude was purified by flash column chromatography (SiO₂: 0-40% EtOAc/Heptane) to get 3-(bis(2-((tert-butyldimethylsilyl)oxy)dodecyl)amino)propyl(4-(bis(2-((tert-butyldimethylsilyl) oxy)dodecyl)amino)butanoyl)-L-tryptophanate [13](0.92 g, 35.85% Yield) as a pale yellow liquid.

### Result:

ELSD analysis: Purity 99.63%, Calculated C₉₀H₁₇₈N₄O₇Si₄=1539.28, Observed=1540.90 (m/z, M+H⁺).

### Synthesis of Compound CI

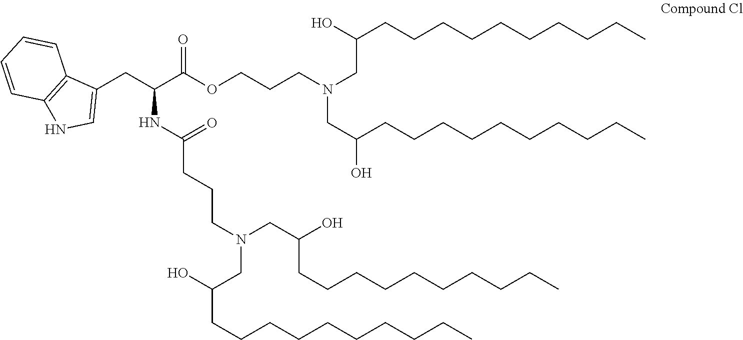 [SMILES: CCCCCCCCCCC(O)CN(CCCOC(=O)[C@H](Cc1c[nH]c2ccccc12)NC(=O)CCCN(CC(O)CCCCCCCCCC)CC(O)CCCCCCCCCC)CC(O)CCCCCCCCCC]

As depicted in Scheme 45: To a stirred solution of 3-(bis{2-[(tert-butyl)bis(methyl)siloxy]dodecyl}amino)propyl 2-[4-(bis{2-[(tert-butyl)bis(methyl)siloxy]dodecyl}amino)butyrylamino]-3-(3-indolyl)propionate [13](0.9 g, 584 μmol) in tetrahydrofuran (10 mL) was added hydrogen fluoride pyridine (2.03 g, 20.4 mmol) dropwise at 0° C. under nitrogen atmosphere. The reaction mixture was stirred at RT for 16 h. The progress of reaction was monitored by TLC. After completion of reaction, reaction mass was diluted with diethyl ether (50 mL) and washed with ice cold water (20 mL), saturated sodium bicarbonate (20 mL), Milli Q water (2×15 mL). The organic layer was collected, dried over sodium sulphate and evaporate under reduced pressure to get 3-(bis(2-hydroxydodecyl)amino)propyl (4-(bis(2-hydroxydodecyl)amino)butanoyl)-L-tryptophanate [Compound CI](540 mg, 85.3% Yield) as a colourless liquid.

### Results:

¹H-NMR (400 MHz, CDCl₃): δ 9.20-8.40 (m, 1H), 7.58 (d, J=8.0 Hz, 1H), 7.38-7.36 (m, 1H), 7.20-7.02 (m, 3H), 4.85-4.81 (m, 1H), 4.31-3.95 (m, 3H), 3.82-3.40 (m, 6H), 3.27-3.26 (m, J=5.2 Hz, 2H), 2.82-2.20 (m, 14H), 1.90-1.60 (m, 4H), 1.49-1.20 (m, 74H), 0.87 (t, J=6.4 Hz, 12H).

ELSD analysis: Purity 98.63%, Calculated C₆₆H₁₂₂N₄O₇=1082.93, Observed=1083.75 (m/z, M+H⁺).

### Example 46: Synthesis of Compound CII

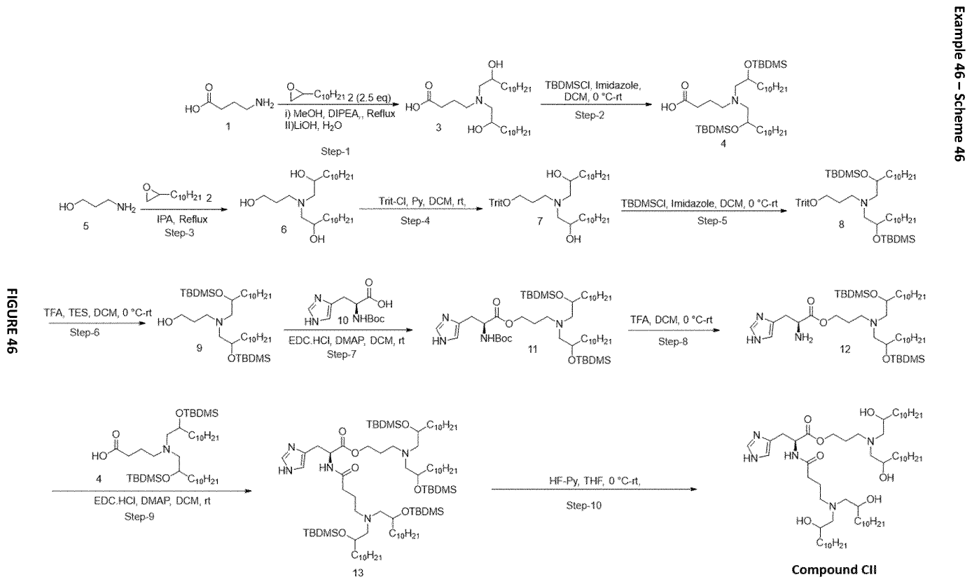

For example, the compounds of the invention may be prepared according to Scheme 46 (as depicted in FIG. 46).

### Intermediate [4] and Intermediate [9]

As depicted in Scheme 46: Synthesis procedure of Intermediate [4] and Intermediate [4] is given in the synthesis for Compound XCV.

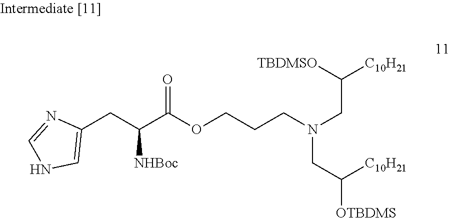

As depicted in Scheme 46: To a stirred solution of (tert-butoxycarbonyl)-L-histidine [10](2 g, 7.83 mmol) in dichloromethane (0.1 L) were added N,N-dimethyl-4-pyridylamine (957 mg, 7.83 mmol) and EDC·HCl (3 g, 15.7 mmol). The reaction mixture was stirred at RT for 30 m in and added 3-(bis{2-[(tert-butyl)bis(methyl)siloxy]dodecyl}amino)-1-propanol [9](4.74 g, 7.05 mmol). The reaction mixture was stirred at room temperature for 16 h. The progress of reaction was monitored by TLC. The reaction mass was diluted with brine solution (3×10 mL) and extracted with DCM (3×50 mL). The combined organic layer was dried over anhydrous sodium sulphate and concentrated under reduced pressure to get crude. The crude was purified by flash column chromatography (SiO₂: 0-70% EtOAc/Heptane) to get 3-(bis(2-((tert-butyldimethylsilyl)oxy) dodecyl)amino)propyl (tert-butoxycarbonyl)-L-histidinate [11](3.5 gm, 49.12% Yield) as a pale yellow liquid.

### Results:

ELSD analysis: Purity 99.48%, Calculated C₅₀H₁₀₀N₄O₆Si₂=908.72, Observed=909.45 (m/z, M+H⁺).

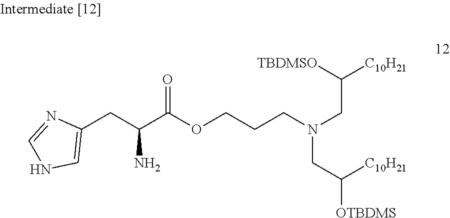

As depicted in Scheme 46: To a stirred solution of 3-(bis(2-((tert-butyldimethylsilyl)oxy)dodecyl)amino)propyl (tert-butoxycarbonyl)-L-histidinate [11](2 g, 2.2 mmol) in dichloromethane (40 mL), was added trifluoroacetic acid (2.53 mL, 33 mmol) at 0° C. The reaction mixture was stirred at room temperature for 3 h. The progress of reaction was monitored by TLC/ELSD. After completion of reaction, reaction mass was quenched with saturated solution of sodium bicarbonate (pH ~8) and extracted with DCM (2×50 mL). The combined organic layer was dried over anhydrous sodium sulphate and concentrated under reduced pressure to get 3-(bis(2-((tert-butyldimethylsilyl)oxy)dodecyl)amino)propyl L-histidinate [12](1.7 gm, 95.51% Yield) as a pale yellow liquid.

### Results:

ELSD analysis: Purity 99.48%, Calculated C₄₅H₉₂N₄O₄Si₂=808.67, Observed=809.45 (m/z, M+H⁺).

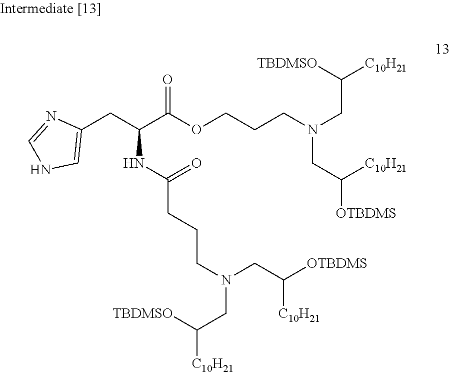

As depicted in Scheme 46: To a stirred solution of 4-(bis{2-[(tert-butyl)bis(methyl)siloxy]dodecyl}amino)butyric acid [4](1.5 g, 2.14 mmol) in dichloromethane (25 mL) were added N,N-dimethyl-4-pyridylamine (131 mg, 1.07 mmol) and EDC·HCl (616 mg, 3.21 mmol) followed by 3-(bis(2-((tert-butyldimethylsilyl)oxy)dodecyl)amino)propyl L-histidinate [11](1.73 g, 2.14 mmol). The reaction mixture was stirred at RT for 16 h. The progress of reaction was monitored by ELSD/TLC. The reaction mass was quenched with water (20 mL) and extracted with DCM (3×30 mL). The organic layers were collected, dried over sodium sulphate and concentrated under reduced pressure to get crude. The crude was purified by flash column chromatography (SiO₂: 0-10% ethyl acetate in hexane) to get 3-(bis(2-((tert-butyldimethylsilyl) oxy)dodecyl)amino)propyl (4-(bis(2-((tert-butyldimethylsilyl)oxy) dodecyl)amino)butanoyl)-L-histidinate [13](850 mg, 26.6% Yield) as a pale yellow liquid.

### Results:

ELSD analysis: Purity 98.14%, Calculated C₈₅H₁₇₅N₅O₇Si₄=1490.26, Observed=1491.70 (m/z, M+H⁺).

### Synthesis of Compound CII

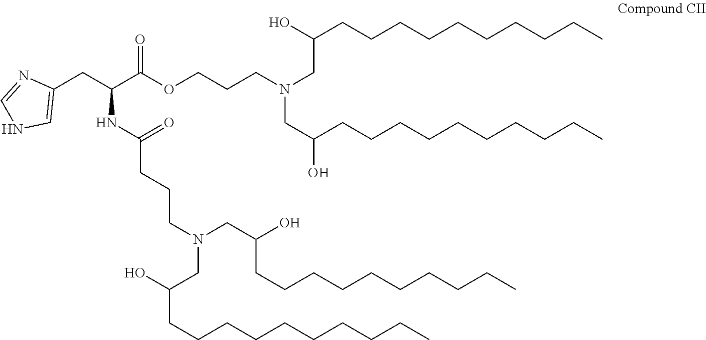 [SMILES: CCCCCCCCCCC(O)CN(CCCOC(=O)[C@H](Cc1c[nH]cn1)NC(=O)CCCN(CC(O)CCCCCCCCCC)CC(O)CCCCCCCCCC)CC(O)CCCCCCCCCC]

As depicted in Scheme 46: To a stirred solution of 3-(bis(2-((tert-butyldimethylsilyl)oxy)dodecyl)amino)propyl (4-(bis(2-((tert-butyldimethylsilyl)oxy)dodecyl)amino)butanoyl)-L-histidinate [13](850 mg, 570 μmol) in tetrahydrofuran (10 mL) was added hydrogen fluoride pyridine (1.98 g, 19.9 mmol) dropwise at 0° C. under nitrogen atmosphere. The reaction mixture was stirred at RT for 16 h. The progress of reaction was monitored by TLC/ELSD. After completion of reaction, reaction mass quenched with saturated sodium bicarbonate solution upto pH-8 and extracted with ethyl acetate (2×15 mL). The organic layers were collected, dried over sodium sulphate and evaporated under reduced pressure to get crude. The crude was purified by reverse phase column (water/0.1% TFA in ACN to get 3-(bis(2-hydroxydodecyl)amino)propyl (4-(bis(2-hydroxydodecyl)amino)butanoyl)-L-histidinate.TFA salt [Compound CII](180 mg, 30.53% Yield) as pale yellow liquid.

### Results:

¹H-NMR (400 MHz, CDCl₃): δ 9.50-9.0 (br, 1H), 8.70-8.40 (m, 1H), 8.40-8.25 (m, 1H), 7.20-7.19 (m, 1H), 4.90-4.68 (m, 1H), 4.44-3.41 (brm, 2H), 4.39-3.90 (brm, 4H), 3.80-3.40 (brm, 8H), 3.39-3.28 (m, 2H), 3.20-3.00 (m, 8H), 2.62-2.32 (brm, 2H), 2.30-2.05 (brm, 2H), 2.02-1.90 (brm, 2H), 1.48-1.40 (m, 8H), 1.35-1.00 (brm, 60H), 0.87 (t, J=6.4 Hz, 12H)

ELSD analysis: Purity 99.67%, Calculated C₆₁H₁₁₉N₅O₇=1033.91, Observed=1035.10 (m/z, M+H⁺).

### Example 47: Synthesis of Compound CIII

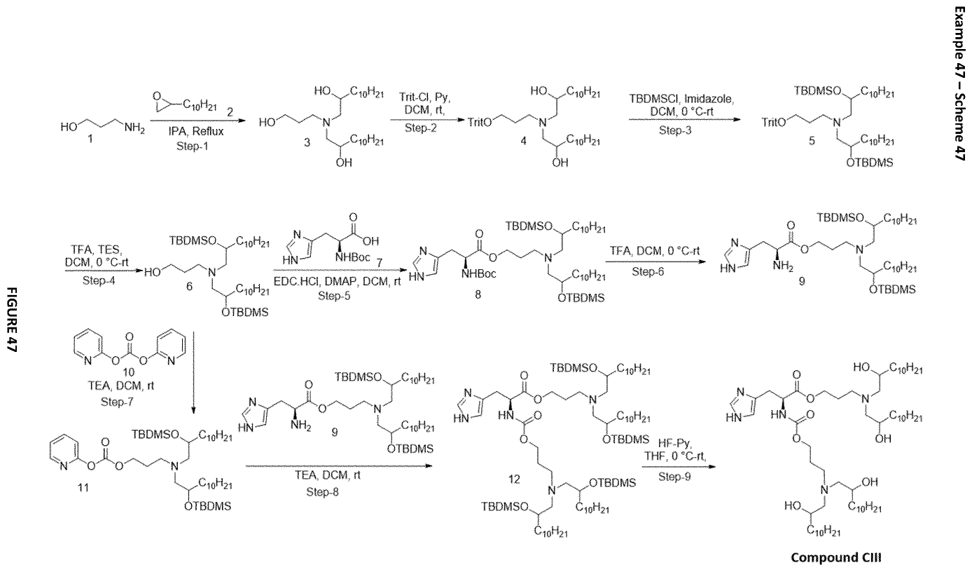

For example, the compounds of the invention may be prepared according to Scheme 47 (as depicted in FIG. 47).

### Intermediate [9]

As depicted in Scheme 47: Synthesis procedure of Intermediate [9] is given in the synthesis for Compound CII.

### Intermediate [11]

As depicted in Scheme 47: Synthesis procedure of Intermediate [11] is given in the synthesis for Compound XCVII.

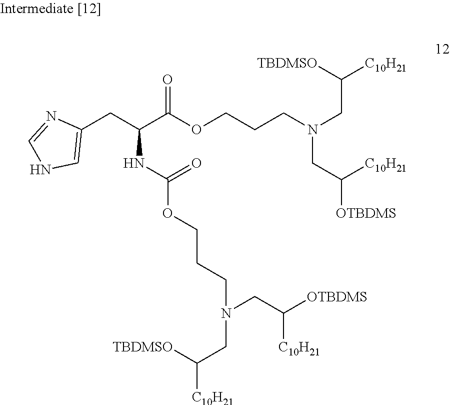

As depicted in Scheme 47: To a stirred solution of 3-(bis{2-[(tert-butyl)bis(methyl)siloxy]dodecyl}amino)propyl 2-pyridyl carbonate [11](1.1 g, 1.39 mmol) in dichloromethane (10 mL, 156 mmol) was added triethylamine (289 μL, 2.08 mmol) and 3-(bis(2-((tert-butyldimethylsilyl) oxy)dodecyl)amino) propyl L-histidinate [9](1.12 g, 1.39 mmol) at room temperature. The reaction mixture was stirred at RT for 16 h. The progress of reaction was monitored by ELSD/TLC. After completion of reaction, reaction mass was concentrated under reduced pressure to get crude. The crude was purified by flash column chromatography (SiO₂: 0-15% Ethyl acetate in heptane) to obtain 3-(bis(2-((tert-butyldimethylsilyl)oxy)dodecyl)amino) propyl(6-(2-((tert-butyldimethylsilyl) oxy)dodecyl)-8-decyl-10,10,11,11-tetramethyl-2,9-dioxa-6-aza-10-siladodecanoyl)-L-histidinate [12](580 mg, 27.75% Yield) as a pale yellow liquid.

### Results:

ELSD analysis: Purity 99.37%, Calculated C₈₅H₁₇₅N₅O₈Si₄=1506.25, Observed=1507.70 (m/z, M+H⁺).

### Synthesis of Compound CIII

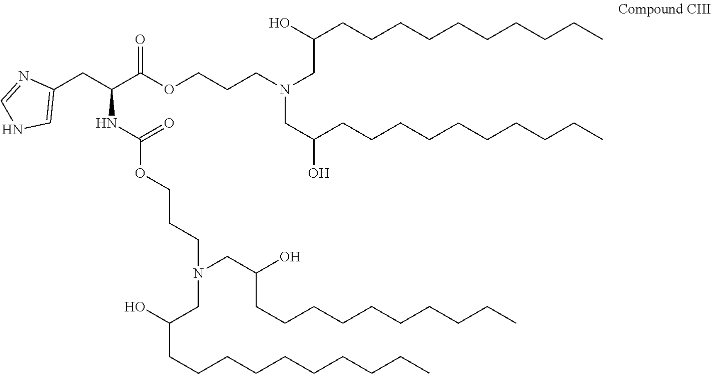 [SMILES: CCCCCCCCCCC(O)CN(CCCOC(=O)N[C@@H](Cc1c[nH]cn1)C(=O)OCCCN(CC(O)CCCCCCCCCC)CC(O)CCCCCCCCCC)CC(O)CCCCCCCCCC]

As depicted in Scheme 47: To a stirred solution of 3-(bis(2-((tert-butyldimethylsilyl)oxy)dodecyl)amino)propyl (6-(2-((tert-butyldimethylsilyl)oxy)dodecyl)-8-decyl-10,10,11,11-tetramethyl-2,9-dioxa-6-aza-10-siladodecanoyl)-L-histidinate [12](550 mg, 365 μmol) in tetrahydrofuran (6 mL, 73.7 mmol) was added hydrogen fluoride pyridine (1.12 mL, 12.4 mmol) at 0° C. under inert atmosphere. The reaction mixture was stirred for 16 h at room temperature. The progress of reaction was monitored by TLC. The reaction mixture was diluted with diethyl ether (50 mL) and washed with cold water (2×5 mL). To the organic layer was added cold saturated sodium bicarbonate solution (pH ~8). The organic layer was separated and washed with cold water (2×5 mL). The organic layer was dried over anhy. sodium sulphate, concentrated under reduced pressure to get crude. The crude was purified by flash column chromatography (SiO₂: 0-20% methanol in DCM) to obtain 3-(bis(2-hydroxydodecyl)amino)propyl ((3-(bis(2-hydroxydodecyl)amino) propoxy)carbonyl)-L-histidinate [Compound CIII](0.3 g, 78.27% Yield) as a pale yellow liquid.

### Results:

¹H-NMR (400 MHz, CDCl₃): δ 7.53 (d, J=4.8 Hz, 1H), 6.81 (s, 1H), 6.40-5.40 (brs, 1H), 4.55 (m, 1H), 4.40-4.00 (m, 4H), 3.62 (brm, 4H), 3.11 (m, 2H), 2.70-2.30 (m, 12H), 1.80-1.60 (m, 4H), 1.50-1.20 (m, 72H), 0.87 (t, J=6.4 Hz, 12H).

ELSD analysis: Purity 99.71%, Calculated C₆₁H₁₁₉N₅O₈=1049.91, Observed=1050.85 (m/z, M+H⁺).

### Example 48: Synthesis of Compound LXXXV

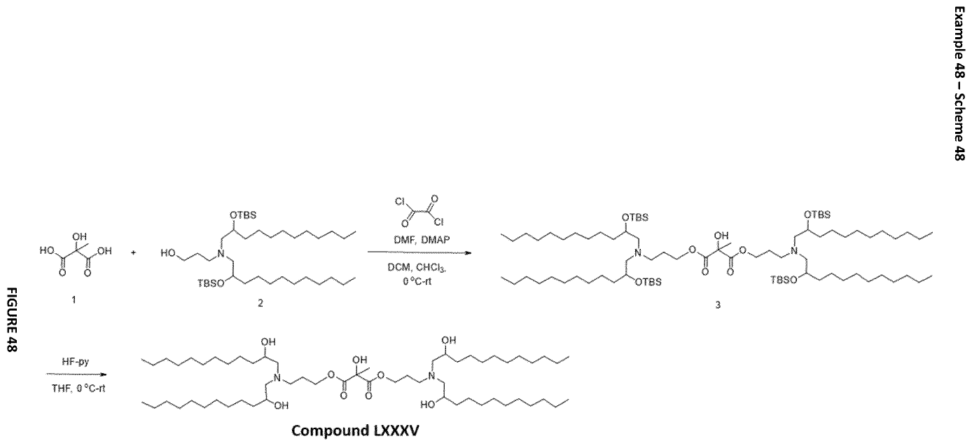

For example, the compounds of the invention may be prepared according to Scheme 48 (as depicted in FIG. 48).

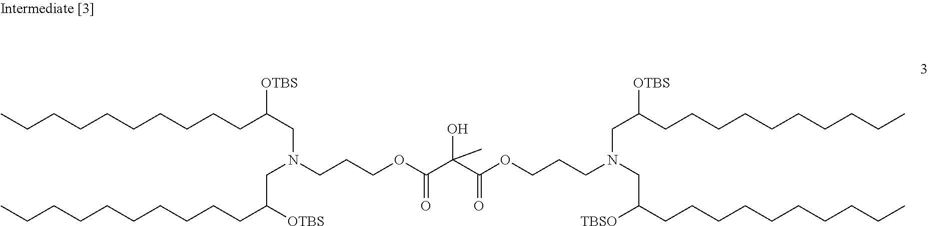 [SMILES: CCCCCCCCCCC(CN(CCCOC(=O)C(C)(O)C(=O)OCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 48: To a 50 mL round bottom flask with a stir bar in an ice bath was added 2-hydroxy-2-methyl-propanedioic acid [1](50 mg, 0.3729 mmol), N,N-dimethylformamide (0.01 mL) and Chloroform (6 mL). The reaction was cooled to 0° C. Then oxalyl dichloride (95 mg, 0.7485 mmol) was added Hydrofluoric Acid via a syringe. The reaction was then stirred at room temp for 16 hours. The reaction mixture was then transferred dropwise using a glass pipet to a 20 mL scintillation vial with a stir bar and prepared solution of 3-[bis[2-[tert-butyl(dimethyl)silyl]oxydodecyl]amino]propan-1-ol [2](550 mg, 0.8181 mmol), 4-Dimethylaminopyridine (190 mg, 1.5552 mmol) and Dichloromethane (7 mL). The coupling reaction was stirred at room temp for 16 hours. LC-MS confirmed the completion of the reaction. The reaction was diluted by Ethyl Acetate then washed by sat. Sodium Bicarbonate and brine and dried by Sodium Sulphate. The organic solvent was removed by reduced pressure and the raw product was purified on a silica column to yield pure intermediate [3](340 mg, 63.2% yield).

### Result:

Calculated C₃₂H₁₇₂N₂O₉Si₄=1441.21, Observed=1459.7 (m/z, M+NH₄⁺).

### Synthesis of Compound LXXXV

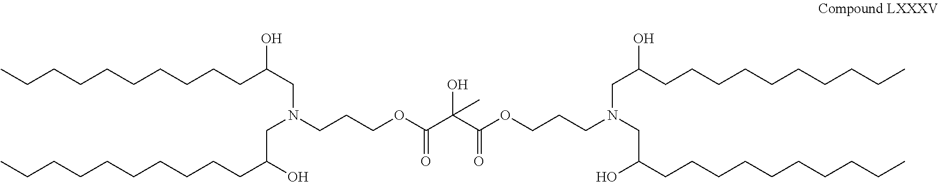 [SMILES: CCCCCCCCCCC(O)CN(CCCOC(=O)C(C)(O)C(=O)OCCCN(CC(O)CCCCCCCCCC)CC(O)CCCCCCCCCC)CC(O)CCCCCCCCCC]

As depicted in Scheme 48: To a 20 mL plastic vial with stir bar was added HMMA-E3E12-TBS [3](340 mg, 0.2357 mmol) and Tetrahydrofuran (6 mL). Then Hydrofluoric Acid (140 mg, 4.8985 mmol, 70% mass) was added via a syringe. The reaction was running at room temp for 16 hours. The LC-MS confirmed the completion of deprotection. The reaction was diluted by Ethyl Acetate and solid Sodium Bicarbonate was added to stir for 30 min. The solution was washed by sat. Sodium Bicarbonate and brine then dried by NaSO4. The organic solvent was dried by reduced pressure and the mixture was purified with silica column to yield pure product as colourless oil (35 mg, 15.1% yield).

### Result:

¹H NMR (400 MHz, CDCl3) δ 4.34-4.11 (m, 4H), 3.41 (s, 4H), 3.20-2.81 (m, 12H), 2.20 (s, 2H), 1.88 (s, 4H), 1.59 (s, 4H), 1.39 (s, 5H), 1.22-1.19 (m, 64H), 0.81 (t, J=6.6 Hz, 12H).

ELSD analysis: Purity 96.9%, Calculated C₅₈H₁₁₆N₂O₉=984.86, Observed=1003.80 (m/z, M+NH₄⁻).

### Example 49: Synthesis of Compound LXXXVI

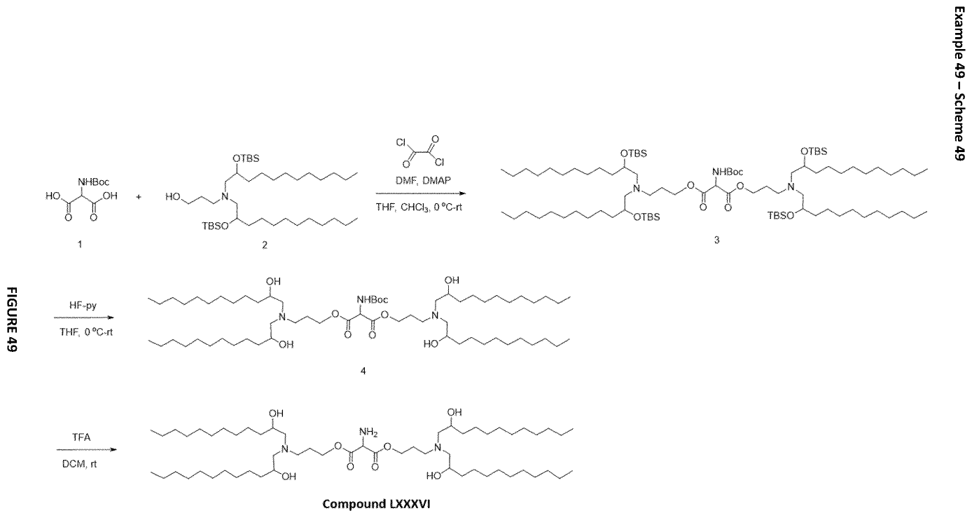

For example, the compounds of the invention may be prepared according to Scheme 49 (as depicted in FIG. 49).

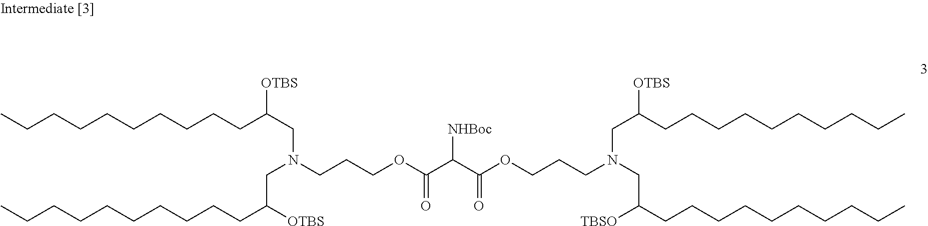 [SMILES: CCCCCCCCCCC(CN(CCCOC(=O)C(NC(=O)OC(C)(C)C)C(=O)OCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 49: To a 50 mL round bottom flask with a stir bar in an ice bath was added 2-(tert-butoxycarbonylamino)propanedioic acid [1](100 mg, 0.4562 mmol), Dimethylformamide (20 μL), Tetrahydrofuran (3 mL) and chloroform (5 mL). Then oxalyl dichloride (B, 100 μL, 1.17 mmol, 100 mass %) was added dropwise via a syringe. The reaction was then stirred at room temp for 16 hours. The reaction mixture was then transferred dropwise using a glass pipet to a 20 mL scintillation vial with a stir bar and prepared solution of 3-[bis[2-[tert-butyl(dimethyl)silyl]oxydodecyl]amino]propan-1-ol [2](700 mg, 1.0413 mmol), 4-Dimethylaminopyridine (180 mg, 1.4734 mmol) and Dichloromethane (7 mL). The coupling reaction was stirred at room temp for 16 hours. LC-MS confirmed the completion of the reaction. The reaction was diluted by Ethyl Acetate then washed by sat. Sodium Bicarbonate and brine and dried by Sodium Sulphate. The organic solvent was removed by reduced pressure and the raw product was purified on a silica column to yield pure intermediate [3](140 mg, 20.1% yield).

### Result:

Calculated C₈₆H₁₇₉N₃O₁₀Si₄=1527.69, Observed=773.6 (m/2z, M+NH₄++H⁺), 1545.2 (m/z, M+NH₄⁺).

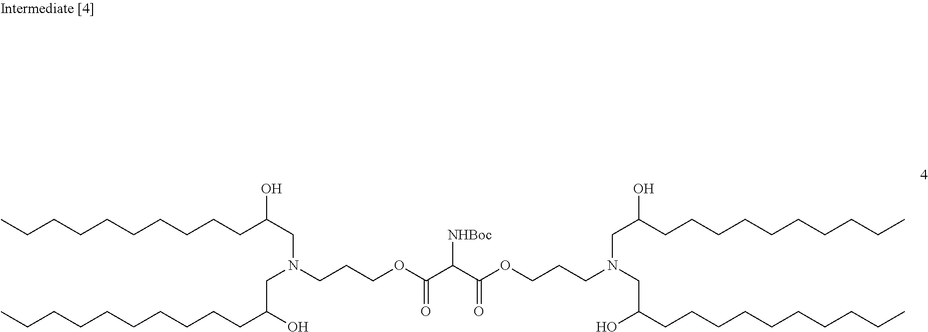 [SMILES: CCCCCCCCCCC(O)CN(CCCOC(=O)C(NC(=O)OC(C)(C)C)C(=O)OCCCN(CC(O)CCCCCCCCCC)CC(O)CCCCCCCCCC)CC(O)CCCCCCCCCC]

As depicted in Scheme 49: To a 20 mL plastic vial with stir bar in an ice batch was added bis[3-[bis[2-[tert-butyl(dimethyl)silyl]oxydodecyl]amino]propyl]2-(tert-butoxycarbonylamino)propanedioate [3](130 mg, 0.0851 mmol) and Tetrahydrofuran (5 mL). Then Hydrofluoric Acid (80 mg, 2.7991 mmol, 70 mass %) was added via a syringe. The reaction was stirred at room temp for 16 hours. The LC-MS confirmed the completion of deprotection. The reaction was diluted by Ethyl Acetate and solid Sodium Bicarbonate was added to stir for 30 min. The solution was washed by sat. Sodium Bicarbonate and brine then dried by Sodium Sulphate. The organic solvent was dried by reduced pressure and the mixture was purified with silica column to yield pure intermediate [4] as colourless oil (56 mg, 61.5% yield).

### Result:

Calculated C₆₂H₁₂₃N₃O₁₀=1069.92, Observed=544.4 (m/2z, M+NH₄⁺+H⁺).

### Synthesis of Compound LXXXVI

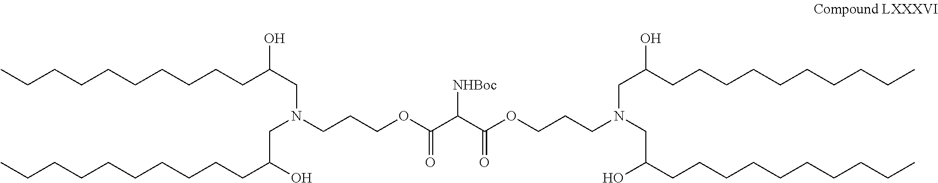 [SMILES: CCCCCCCCCCC(O)CN(CCCOC(=O)C(NC(=O)OC(C)(C)C)C(=O)OCCCN(CC(O)CCCCCCCCCC)CC(O)CCCCCCCCCC)CC(O)CCCCCCCCCC]

As depicted in Scheme 49: To a 20 mL scintillation vial with stir bar was added NHBocMA-E3E12 [4](56 mg, 0.0523 mmol) and Dichloromethane (5 mL). Then Trifluoroacetic Acid (50 mg, 0.4373 mmol) was added via a syringe. The reaction was stirred at room temp for 48 hours. Then Dichloromethane was removed under reduced pressure and the mixture was diluted in Ethyl Acetate. Then the solution was washed by sat. Sodium Bicarbonate and brine then dried by Sodium Sulphate. The organic phase was collected, and the solvent was removed under reduced pressure. The crude product was purified on a silica column to yield oil as pure product (25 mg, 45.3% yield).

### Result:

¹H NMR (400 MHz, CDCl3) δ 5.21 (s, 1H), 4.03 (t, J=7.0 Hz, 4H), 3.82-3.50 (m, 4H), 2.57-2.25 (m, 12H), 1.95 (d, J=3.3 Hz, 4H), 1.76-1.53 (m, 8H), 1.37-1.18 (m, 64H), 0.80 (d, J=5.3 Hz, 12H) ppm.

ELSD analysis: Purity 99.7%, Calculated C₅₇H₁₁₅N₃O₈=969.86, Observed=970.90 (m/z, M+H⁺).

### Example 50: Synthesis of Compound LXXXIV

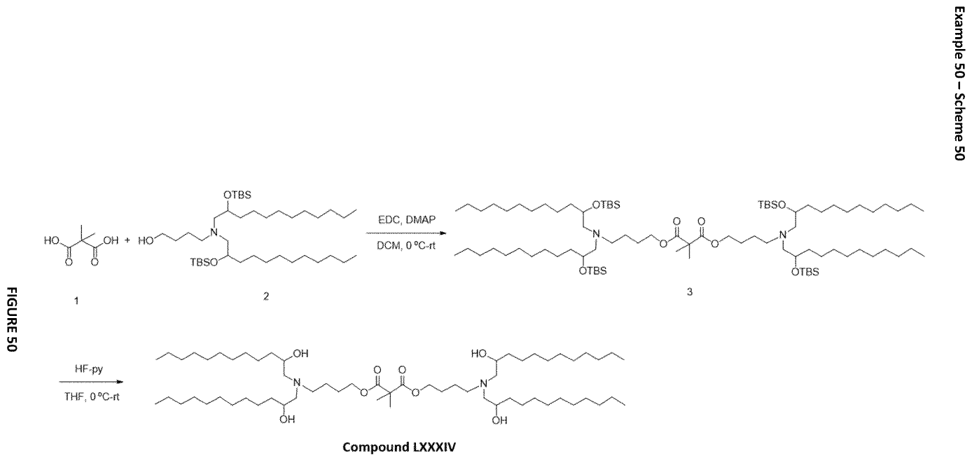

For example, the compounds of the invention may be prepared according to Scheme 50 (as depicted in FIG. 50).

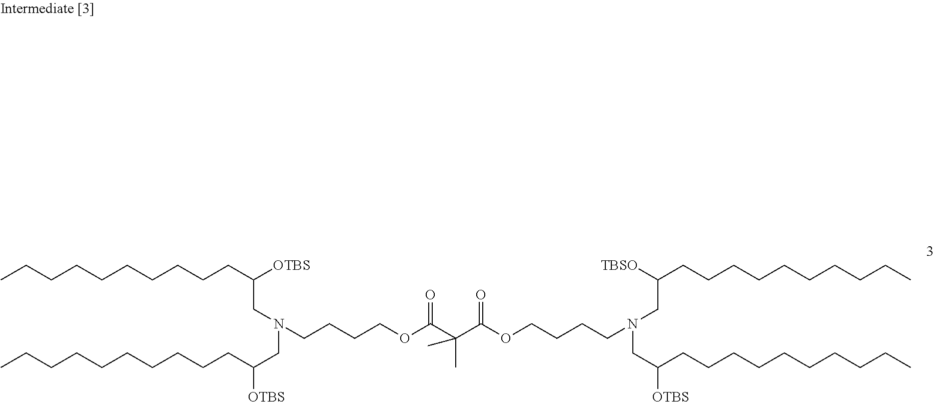 [SMILES: CCCCCCCCCCC(CN(CCCCOC(=O)C(C)(C)C(=O)OCCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 50: To a 50 ml round bottom flask equipped with a stir bar was added 2,2-dimethylpropanedioic acid [1](100 mg, 0.7569 mmol), HIM-E4E12 [2](1.20 g, 1.75 mmol), 4-Dimethylaminopyridine (180 mg, 1.4734 mmol) and Dichloromethane (12 mL), purged with nitrogen. 1-Ethyl-3-(3-dimethylaminopropyl)carbodiimide (350 mg, 1.8258 mmol) was added and the reaction was stirred for 18 hours. Completion of the reaction was determined by LC/MS. The solvent was removed by reduced pressure and the reaction mixture was dissolved in Ethyl Acetate. The organic solution was then washed by water, brine and dried by sodium sulphate. The organic phase was filtered and solvent was removed by reduced pressure. The crude product was purified by silica column with Ethyl Acetate/Hexane as mobile phase to yield pure intermediate [3](200 mg, 18.0% yield).

### Result:

Calculated C₈₅H₁₇₈N₂O₈Si₄=1467.27, Observed=1468.33 (m/z, M+H⁺).

### Synthesis of Compound LXXXIV

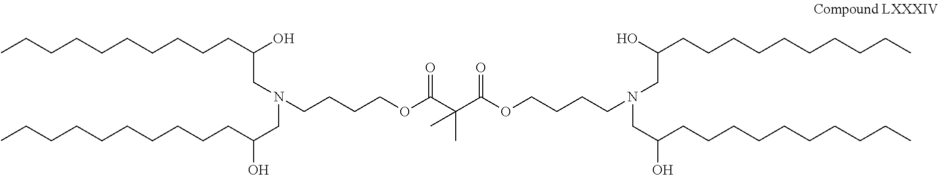 [SMILES: CCCCCCCCCCC(O)CN(CCCCOC(=O)C(C)(C)C(=O)OCCCCN(CC(O)CCCCCCCCCC)CC(O)CCCCCCCCCC)CC(O)CCCCCCCCCC]

As depicted in Scheme 50: To a 50 ml plastic round bottom flask equipped with a stir bar was added DMMA-E4E12-TBS [3](190 mg, 0.1294 mmol) along with Tetrahydrofuran (5 mL). The reaction was purged with nitrogen and then cooled down to 0° C. using an ice bath. Then while stirring, Hydrofluoric Acid (60 mg, 2.0993 mmol, 70 mass %) was added and allowed to stir for 5 min at 0° C. and then at room temperature overnight. The reaction was checked for completion using LC/MS. The reaction mixture was diluted by Ethyl Acetate and was added solid sodium bicarbonate to stir for 30 min. Then the mixture was washed with saturated sodium bicarbonate solution, brine and dried by magnesium sulphate.

The organic phase was filtered and concentrated under reduced pressure. The crude product was purified by silica column with Ethyl Acetate/Hexane as mobile phase to yield colourless oil as final product (72 mg, 55.0% yield).

### Result:

1H NMR (400 MHz, CDCl3) δ 4.01 (s, 4H), 3.65 (s, 4H), 2.74-2.16 (m, 12H), 1.55 (m, 4H), 1.36 (m, 4H), 1.20 (s, 72H), 1.14-0.95 (m, 12H), 0.81 (q, J=4.8 Hz, 12H) ppm.

ELSD analysis: Purity 97.1%, Calculated C₆₁H₁₂₂N₂O₈=1010.92, Observed=1011.01 (m/z, M+H⁺).

### Example 51: Lipid Nanoparticule Formulation

Cationic lipids described herein can be used in the preparation of lipid nanoparticles according to methods known in the art. For example, suitable methods include methods described in International Publication No. WO 2018/089801, which is hereby incorporated by reference in its entirety.

The lipid nanoparticles in the examples of the present invention were formulated using Process A of WO 2018/089801 (see, e.g., Example 1 and FIG. 1 of WO 2018/089801). Process A (“A”) relates to a conventional method of encapsulating mRNA by mixing mRNA with a mixture of lipids, without first pre-forming the lipids into lipid nanoparticles. In an exemplary process, an ethanol lipid solution and an aqueous buffered solution of mRNA were prepared separately. A solution of mixture of lipids (cationic lipid, helper lipids, zwitterionic lipids, PEG lipids etc.) was prepared by dissolving lipids in ethanol. The mRNA solution was prepared by dissolving the mRNA in citrate buffer. Then, these two solutions were mixed using a pump system. In some instances, the two solutions were mixed using a gear pump system. In certain embodiments, the two solutions were mixing using a ‘T’ junction (or “Y” junction). The mixture was then purified by diafiltration with a TFF process. The resultant formulation concentrated and stored at 2-8° C. until further use.

Lipid nanoparticle formulations of Table 1 were prepared by Process A. More specifically, an ethanolic solution of a mixture of lipids including DMG-PEG-2000, cationic lipid, cholesterol, and phosphatidylethanolamine (DOPE) in a ratio of 1.5:40:28.5:30 were combined with an aqueous buffered solution (1 mM Citrate and 150 mM NaCl) of target mRNA at an acidic pH (4.5) under controlled conditions to yield a dispersion of uniform lipid nanoparticles (LNPs). Upon ultrafiltration and diafiltration into the final buffer, the resulting nanoparticle dispersions were diluted to required concentration using the final buffer (10% Trehalose), sterile filtered and stored frozen at −80° C. until use.

All of the lipid nanoparticle formulations comprised hEPO mRNA and the different lipids in following mol % ratios: Cationic Lipid: DMG-PEG2000; Cholesterol:DOPE=40:1.5:28.5:30.

DLS measurements were performed using a Malvern Instruments Zetasizer with a backscattering detector angle of 173° and a 4-mW, 633-nm He—Ne laser (Worcestershire, UK). The samples were analyzed by diluting in 10% Trehalose and measuring the size and Poly dispersity Index (PDI) in an optical grade polystyrene cuvette.

TABLE 1
Exemplary lipid nanoparticle characterizations

| Compound | | Size | | Encapsulation |
|---|---|---|---|---|
| # | N/P ratio | (nm) | PDI | Percentage (%) |
| XII | 4 | 110 | 0.091 | 46 |
| XXV | 4 | 89 | 0.087 | 83 |
| XXXII | 4 | 106 | 0.124 | 78 |
| XXXVIII | 4 | 101 | 0.089 | 64 |
| XV | 4 | 134 | 0.052 | 33 |
| XIV | 4 | 101 | 0.098 | 67 |

The N/P ratio is defined as the ratio of the number of nitrogen in cationic lipid to the number of phosphate in nucleic acid.

### Example 52: Delivery of hEPO mRNA by Intramuscular Administration

WO2022/099003 A1 describes an in vivo assay for intramuscular administration (e.g. on page 46, paragraph [00206]). Further details of the intramuscular experiment performed in this application are provided below.

Study Design Table

| | | | | | Dose | | |
|---|---|---|---|---|---|---|---|
| Group | No. of | | Dose Levels | Conc. | Volume | Dosing Regimen | Terminal |
| No. | Animals | Test Article | (μg/An.) | (μg/mL) | (μL/An.) | ROA | Time Point |
| 1 | 4 | SALINE | 0.0 | 0.0 | 30 | Once on Day 1 | At 24 hours |
| 2 | 4 | DLIN-MC3- | 0.1 | 3.33 | | via IM injections | post dose on |
| | | DMA (MC3) | | | | into the right | Day 2 |
| 3 | 4 | XII | 0.1 | 3.33 | | gastrocnemius | |
| 4 | 4 | XXV | 0.1 | 3.33 | | muscle. | |
| 5 | 4 | XXXII | 0.1 | 3.33 | | | |
| 6 | 4 | XXXVIII | 0.1 | 3.33 | | | |
| 7 | 4 | XV | 0.1 | 3.33 | | | |
| 8 | 4 | XIV | 0.1 | 3.33 | | | |

An. = animal; TA = test article; Conc. = concentration; ROA = route of administration; IM = intramuscular.

### Test Materials and Treatment Regimen

Test materials remained RNase free during loading into the syringe (as applicable).

### Test Article Class of Compound: Oligonucleotides

### ABSL-1

Treatment Regimen: On Day 1, animals from Groups 1-8 were dosed via intramuscular injection while under light isoflurane anesthesia according to the study design table above. Animals in Groups 2-8 were injected with EPO in the right leg only. Group 1 animals received saline control.

### Study Animals

### Animals:

| | |
|---|---|
| Species/Sex | Mouse/Female |
| Strain | BALB/cJ (Jax #000651) |
| Number | N = 32 + 4 spares |
| Age | 6-8 Weeks |

Acclimation: Animals were acclimatised to the Test Facility for at least 24 hours.

Housing: All animals were socially housed in polycarbonate cages with contact bedding in an animal housing room.

Food and Water: Food (Envigo irradiated 2918 diet) and filtered tap water was provided to animals ad libitum.

### In-Life Observations and Measures

Animal Health Checks: At least once daily animals received a cage side health check observation.

Clinical Observations: Clinical observations were performed for all animals on Day 1 prior to dose administration and prior to euthanasia. Clinical observations were performed more often if abnormal clinical signs were exhibited by animals on study.

Body Weights: Body weights were recorded prior to test material administration. Body weights were rounded to the nearest 0.1 g.

Interim Sample Collections: Interim whole blood (~50 μL) was collected by tail snip or saphenous vein at 6 hours post dose administration (±5%). Blood samples were collected into serum separator tubes, allowed to clot at room temperature for at least 10 minutes, centrifuged at ambient temperature at minimum 1000 g for 10 minutes and the serum was extracted. All serum samples were stored at nominally −70° C. until analysis hEPO by the Testing Facility. The results of the EPO analysis were included in the Data Submission.

In-Life Sample Collection Table

| Group | In Life Sample Collection Time Points |
|---|---|
| No. | Whole Blood |
| 1-8 | T = 6 Hours Post Dose |
| Volume | ~50 μL |
| Additive | None |

No. = number

### Terminal Procedures

Euthanasia: On Day 2, 24 hours post dose, all animals were euthanized by CO₂ asphyxiation followed by thoracotomy and terminal blood collection.

Terminal Blood Collections: Whole blood was collected via cardiac puncture into serum separator tube, allowed to clot at room temperature for at least 10 minutes, centrifuged at ambient temperature at minimum 1000 g for 10 minutes and the serum was extracted. Serum samples were stored at nominally −70° C. until analyzed for hEPO by the Test Facility.

Terminal Sample Collection Table

| Group | Terminal Sample Collection Time Points |
|---|---|
| No. | Whole Blood |
| 1-8 | T = 24 Hours Post Dose |
| Volume | MOV |
| Additive | None |

No. = number; MOV = maximum obtainable volume.

### In-Vitro Assays:

ELISA Assay: Human erythropoietin (hEPO) levels in sera samples were determined by ELISA kit (R&D systems, Cat #DEP-00) according to the manufactory instruction and the results were included in the Data Submission. The “shaker” protocol was used. The serum samples were diluted between 1:40 and 1:100.

### Reporting and Data Retention

Data Submission: A tabulated data summary of animal assignment, individual and group means (as applicable) for times of dose administration and euthanasia, body weights, clinical observations in-vitro analysis and mortality (as applicable) were delivered for this study.

TABLE 2
Results of hEPO mRNA delivery studies - intramuscular
administration of hEPO mRNA lipid formulations
comprising the claimed cationic lipids.

| | hEPO 6 Hr | hEPO 6 Hr | hEPO 24 Hr | hEPO 24 Hr |
|---|---|---|---|---|
| | (ng/mL) | (ng/mL) | (ng/mL) | (ng/mL) |
| Compound # | Mean | SD | Mean | SD |
| XII | 35.0 | 7.3 | 15.1 | 2.4 |
| XXV | 26.8 | 9.6 | 12.2 | 2.0 |
| XXXII | 17.0 | 3.2 | 10.3 | 0.3 |
| XXXVIII | 49.3 | 7.5 | 28.2 | 3.0 |
| XV | 31.9 | 2.6 | 15.1 | 1.3 |
| XIV | 33.9 | 6.5 | 18.3 | 3.6 |

### Example 53: RiboGreen Assay

The RiboGreen Assay is a fluorescence-based method for the determination of mRNA concentration (Total and Free) and % encapsulation using Quant-iT™ RiboGreen® RNA reagent in mRNA containing lipid nanoparticles.

### Materials/Reagents

Triton-X, 98%, for molecular biology, DNAse, RNAse and Protease free, Acros Organics, Cat. AC327371000
UltraPure DNase/RNase-free Distilled Water Life Technologies, Cat. 10977-023
RNaseZap® RNase Decontamination Solution Life Technologies, Cat. AM9784
Quant-iT™ RiboGreen® RNA Reagent Life Technologies, Cat. R11491 or Quant-iT™ RiboGreen® RNA Assay Kit Life Technologies, Cat. R11490
RNase free 20×TE Buffer Life Technologies, Cat. T11493
RNaseZap® RNase Decontamination Solution Life Technologies, Cat. AM9784

### Equipment

Molecular Devices Gemini EM Microplate Reader
RNase Free Microcentrifuge Tubes (2.0 mL)
RNase Free Flacon Tubes (15 and 50 mL)
Vortex mixer
Corning® 96 Well Special Optics Microplate with Clear Background (Cat #3615)
   Preparation of mRNA Standards

mRNA Dilution

| | |
|---|---|
| 50X | 2X |

| | | | | |
|---|---|---|---|---|
| 10X TE buffer | 950 | μL | 1920 | μL |
| mRNA | 50 | μL | 80 | μL |

| | 0 | 0.02 μg/mL | 0.05 μg/mL | 0.2 μg/mL | 0.4 μg/mL | 0.6 μg/mL |
|---|---|---|---|---|---|---|
| Standards | blank | mRNA-1 | mRNA-2 | mRNA-3 | mRNA-4 | mRNA-5 |
| 10X TE buffer | 950 μL | 930 μL | 900 μL | 750 μL | 550 μL | 350 μL |
| 4% Triton | 50 μL | 50 μL | 50 μL | 50 μL | 50 μL | 50 μL |
| 2X mRNA dil. | 0 μL | 20 μL | 50 μL | 200 μL | 400 μL | 600 μL |
| 200-Fold RG | 1000 μL | 1000 μL | 1000 μL | 1000 μL | 1000 μL | 1000 μL |

### Sample Preparation

Sample Dilution

| | 100X | |
|---|---|---|
| 10X TE buffer | 990 | μL |
| LNP sample | 10 | μL |

Samples

| | |
|---|---|
| Total mRNA | Free mRNA |

| | | | | |
|---|---|---|---|---|
| 10X TE buffer | 900 | μL | 750 | μL |
| 4% Triton | 50 | μL | 0 | μL |
| LNP sample dil. (100x) | 50 | μL | 250 | μL |
| 200-Fold RG | 1000 | μL | 1000 | μL |

### 200-Fold RiboGreen Dye Preparation

200-Fold RiboGreen

| | |
|---|---|
| # tubes*5 uL = Amount of | Ex. (10 tubes + 2extra) *5 μL = |
| RG in (#tube)mL TE | 60 μL RG in 12 mL TE |

### Procedure

To each of the standards (Blank, mRNA-1, mRNA-2. mRNA-3, mRNA-4, mRNA-5) and Samples (free mRNA and total mRNA), add 1.0 mL of 200-fold Ribogreen Reagent Solution and gently mix by inversion. This is a 2× Dilution.
Add 200 μL of each standard and sample in triplicate using the reverse pipetting technique in a 96-well Costar Black with Clear Background Plate. Ensure no bubbles are present in the plate before the fluorescence reading.
Read the fluorescence signal using the below instrument parameters:
Read Type: Fluorescence, Bottom Read
Excitation: 485 nm; Cut-off: 515 nm; Emission: 530 nm
Plate Type: 96-well Costar Black with Clear Background

### Data Analysis

The average fluorescence from each calibration standard is plotted against the concentration to generate a linear calibration curve using the MS Excel software. The coefficient of determination (R²) of calibration curve must be R²>0.99.

The linear equation generated can be interpreted as follows:

$$y = mx + c$$

Where,
Y=average fluorescence value
m: slope
x: concentration (pg/mL)
c: y-intercept
Using the linear equation, calculate the concentration of free and total mRNA concentration in the test sample by replacing the y value in the equation with the average fluorescence value of each respective sample
Once the concentration is determined, the actual concentration in the sample can be back-calculated by multiplying the concentration in the test sample with the dilution factor (DF) as follows:

$$Free mRNA Conc . = Conc . of Free mRNA in Test Sample X 800 ( DF ) \\ Total mRNA Conc . = Conc . of Total mRNA in Test Sample X 4000 ( DF )$$

Concentration of encapsulated mRNA can be determined by subtracting the concentration of free mRNA from the total mRNA.
% Encapsulation can then be calculated by taking the ratio of encapsulated mRNA over total mRNA and multiplying the result with 100.

### Example 54: Formulation of Further Lipid Nanoparticules

The following lipid nanoparticles were formulated according to Example 51.

Lipid nanoparticle formulations of Table 3 were prepared by Process A. More specifically, an ethanolic solution of a mixture of lipids including DMG-PEG-2000, cationic lipid, cholesterol, and phosphatidylethanolamine (DOPE) in a ratio of 1.5:40:28.5:30 were combined with an aqueous buffered solution (1 mM Citrate and 150 mM NaCl) of target mRNA at an acidic pH (4.5) under controlled conditions to yield a dispersion of uniform lipid nanoparticles (LNPs). Upon ultrafiltration and diafiltration into the final buffer, the resulting nanoparticle dispersions were diluted to required concentration using the final buffer (10% Trehalose), sterile filtered and stored frozen at −80° C. until use.

All of the lipid nanoparticle formulations comprised hEPO mRNA and the different lipids in following mol % ratios: Cationic Lipid: DMG-PEG2000; Cholesterol:DOPE=40:1.5:28.5:30.

TABLE 3
Further exemplary lipid nanoparticle characterizations

| Compound | | Size | | Encapsulation |
|---|---|---|---|---|
| # | N/P ratio | (nm) | PDI | Percentage (%) |
| LXXXVIII | 4 | 100 | 0.134 | 84 |
| LXXXVII | 4 | 123 | 0.111 | 88 |
| CI | 4 | 135 | 0.155 | 85 |
| XCIX | 4 | n/a | n/a | n/a |
| XCVII | 4 | 125 | 0.101 | 83 |
| XCVI | 4 | 123 | 0.166 | 85 |
| C | 4 | 108 | 0.093 | 82 |
| XCV | 4 | 94 | 0.191 | 66 |
| CIII | 4 | 93 | 0.126 | 91 |
| LXXVII | 4 | 114 | 0.137 | 99 |
| XCIV | 4 | 138 | 0.085 | 96 |

The N/P ratio is defined as the ratio of the number of nitrogen in cationic lipid to the number of phosphate in nucleic acid.

### Example 55: Further Delivery of hEPO mRNA by Intramuscular Administration

The ability of the further exemplary lipid nanoparticles in Example 54 to deliver hEPO mRNA by intramuscular administration was tested in accordance with the experimental protocol set out in Example 52. The protocol in Example 52 was performed on five separate occasions and the results of each occasion are shown in Tables 4-8

The Study Design Table for the further exemplary lipid nanoparticles is shown below.

Study Design Table for the further exemplary lipid nanoparticles

| | | | Dose | | Dose | | |
|---|---|---|---|---|---|---|---|
| Group | No. of | | Levels | Conc. | Volume | Dosing Regimen | Terminal |
| No. | Animals | Test Article | (μg/An.) | (μg/mL) | (μL/An.) | ROA | Time Point |
| First Occasion | | | | | 30 | Once on Day 1 | At 24 hours |
| 9 | 4 | LXXXVIII | 0.1 | 3.33 | | via IM injections | post dose on |
| 10 | 4 | LXXXVII | 0.1 | 3.33 | | into the right | Day 2 |
| Second Occasion | | | | | | gastrocnemius | |
| 11 | 4 | CI | 0.1 | 3.33 | | muscle. | |
| Third Occasion | | | | | | | |
| 12 | 4 | XCIX | 0.1 | 3.33 | | | |
| 13 | 4 | XCVII | 0.1 | 3.33 | | | |
| Fourth Occasion | | | | | | | |
| 14 | 4 | XCVI | 0.1 | 3.33 | | | |
| 15 | 4 | C | 0.1 | 3.33 | | | |
| 16 | 4 | XCV | 0.1 | 3.33 | | | |
| 17 | 4 | CIII | 0.1 | 3.33 | | | |
| Fifth Occasion | | | | | | | |
| 18 | 4 | LXXVII | 0.1 | 3.33 | | | |
| 19 | 4 | XCIV | 0.1 | 3.33 | | | |

An. = animal; TA = test article; Conc. = concentration; ROA = route of administration; IM = intramuscular.

The results of the hEPO mRNA delivery studies of each occasion are set out in Tables 4-8 below.

TABLE 4
Results of hEPO mRNA delivery studies for the first occasion -
intramuscular administration of hEPO mRNA lipid formulations
comprising the claimed cationic lipids.

| Compound | hEPO 6 Hr (ng/mL) | hEPO 6 Hr (ng/mL) |
|---|---|---|
| # | Mean | SD |
| LXXXVIII | 51.7 | 11.1 |
| LXXXVII | 29.1 | 2.8 |

TABLE 5
Results of hEPO mRNA delivery studies for the second occasion -
intramuscular administration of hEPO mRNA lipid formulations
comprising the claimed cationic lipids.

| Compound | hEPO 6 Hr (ng/mL) | hEPO 6 Hr (ng/mL) |
|---|---|---|
| # | Mean | SD |
| CI | 40.5 | 9.4 |

TABLE 6
Results of hEPO mRNA delivery studies for the third occasion -
intramuscular administration of hEPO mRNA lipid formulations
comprising the claimed cationic lipids.

| Compound | hEPO 6 Hr (ng/mL) | hEPO 6 Hr (ng/mL) |
|---|---|---|
| # | Mean | SD |
| XCIX | 38.1 | 1.8 |
| XCVII | 16.0 | 2.0 |

TABLE 7
Results of hEPO mRNA delivery studies for the fourth occasion -
intramuscular administration of hEPO mRNA lipid formulations
comprising the claimed cationic lipids.

| Compound | hEPO 6 Hr (ng/mL) | hEPO 6 Hr (ng/mL) |
|---|---|---|
| # | Mean | SD |
| XCVI | 37.3 | 14.3 |
| C | 37.2 | 3.6 |
| XCV | 22.4 | 6.8 |
| CIII | 15.4 | 5.1 |

TABLE 8
Results of hEPO mRNA delivery studies for the fifth occasion -
intramuscular administration of hEPO mRNA lipid formulations
comprising the claimed cationic lipids.

| Compound | hEPO 6 Hr (ng/mL) | hEPO 6 Hr (ng/mL) |
|---|---|---|
| # | Mean | SD |
| LXXVII | 45.6 | 8.4 |
| XCIV | 36.4 | 5.5 |

From the foregoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

### Numbered Embodiments

1. A cationic lipid having a structure according to Formula (I):

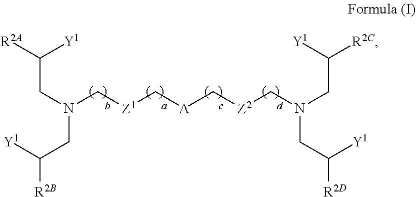

or a pharmaceutically acceptable salt thereof,
   wherein A is selected from —N(R¹)— or —S—S—;
      R¹ is optionally substituted (C₁-C₆)alkyl;
      a and c are integers that are each independently selected from 1, 2, 3 or 4;
      b and d are integers that are each independently selected from 1, 2, 3, 4, 5 or 6;
      Z¹ is selected from a covalent bond,

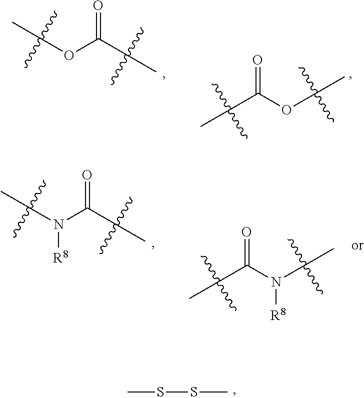

wherein the left hand side of each depicted structure is bound to the —(CH₂)_{b}—;
Z² is selected from a covalent bond,

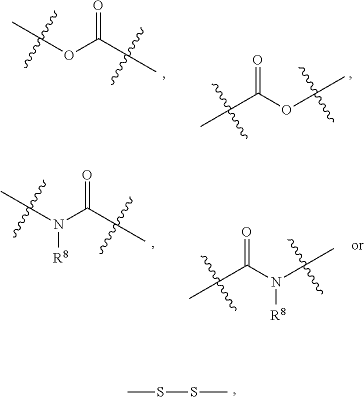

wherein the right hand side of each depicted structure is bound to the —(CH₂)_{d}—;
each Y¹ is independently selected from hydrogen or —OH;
each R⁸ is independently selected from hydrogen or optionally substituted (C₁-C₆)alkyl;
R^{2A}, R^{2B}, R^{2C}, and R^{2D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y¹)—when W¹ is a covalent bond.
2. The cationic lipid of numbered embodiment 1, wherein R¹ is selected from (C₁-C₆)alkyl, or (C₁-C₆)alkylene-R^{A}, wherein
   R^{A} is selected from —OH, —N(R⁶)(R⁷), or

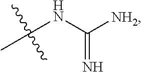

wherein each R⁶ and R⁷ is independently selected from optionally substituted (C₁-C₆)alkyl.
3. The cationic lipid of numbered embodiment 1 or 2, wherein the cationic lipid has a structure according to Formula (IA):

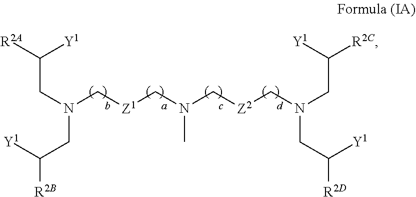

or a pharmaceutically acceptable salt thereof.
4. The cationic lipid of any one of numbered embodiments 1-3, wherein the cationic lipid has a structure according to Formula (IA1):

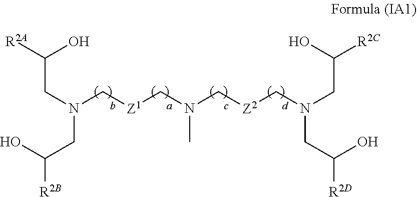

or a pharmaceutically acceptable salt thereof.
5. The cationic lipid of any one of numbered embodiments 1-4, wherein the cationic lipid has a structure according to Formula (IA1i):

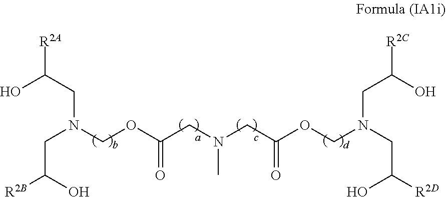

or a pharmaceutically acceptable salt thereof.
6. The cationic lipid of any one of numbered embodiments 1-5, wherein the cationic lipid has a structure according to Formula (IA1ia):

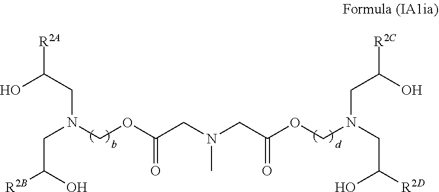

or a pharmaceutically acceptable salt thereof.
7. The cationic lipid of any one of numbered embodiments 1-3, wherein the cationic lipid has a structure according to Formula (IA2):

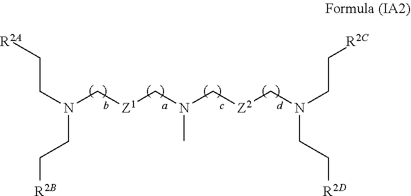

or a pharmaceutically acceptable salt thereof.
8. The cationic lipid of numbered embodiment 1 or 2, wherein the cationic lipid has a structure according to Formula (IB):

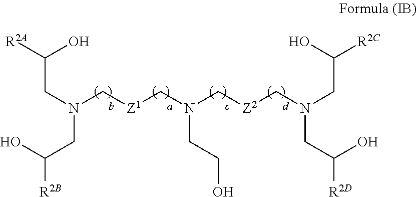

or a pharmaceutically acceptable salt thereof.
9. The cationic lipid of numbered embodiment 1 or 2, wherein the cationic lipid has a structure according to Formula (IC):

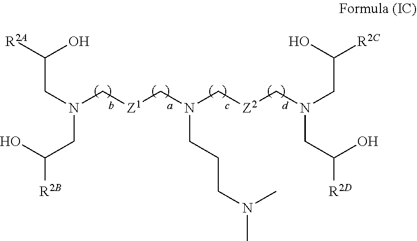

or a pharmaceutically acceptable salt thereof.
10. The cationic lipid of numbered embodiment 1 or 2, wherein the cationic lipid has a structure according to Formula (ID):

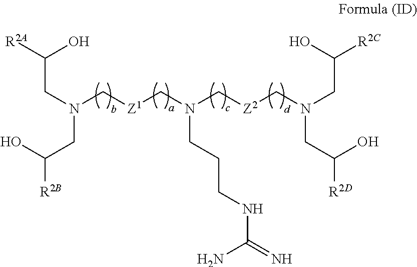

or a pharmaceutically acceptable salt thereof.
11. The cationic lipid of numbered embodiment 1, wherein the cationic lipid has a structure according to Formula (IE):

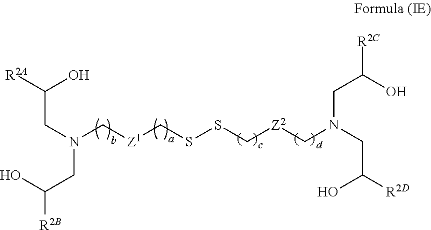

or a pharmaceutically acceptable salt thereof.
12. The cationic lipid of any one of numbered embodiments 1-4, wherein the cationic lipid has a structure according to Formula (IA1ii):

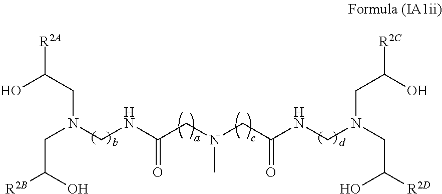

or a pharmaceutically acceptable salt thereof.
13. The cationic lipid of any one of numbered embodiments 1-4 or 12, wherein the cationic lipid has a structure according to Formula (IA1iia):

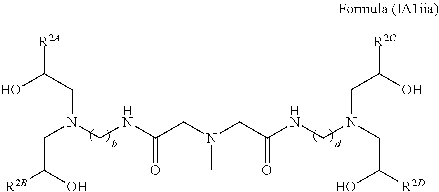

or a pharmaceutically acceptable salt thereof.
14. The cationic lipid of any one of numbered embodiments 1-4, wherein the cationic lipid has a structure according to Formula (IA1iii):

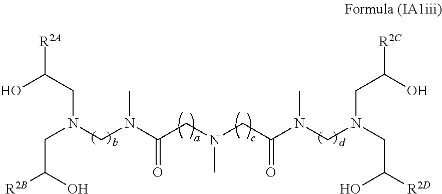

or a pharmaceutically acceptable salt thereof.
15. The cationic lipid of any one of numbered embodiments 1-4 or 14, wherein the cationic lipid has a structure according to Formula (IA1iiia):

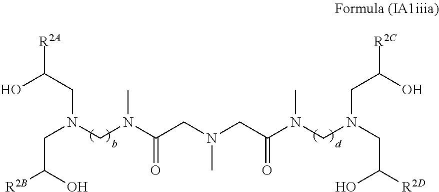

or a pharmaceutically acceptable salt thereof.
16. The cationic lipid of any one of numbered embodiments 1-3, wherein Y¹ is —OH.
17. The cationic lipid of any one of numbered embodiments 1-4, 7-11, or 16, wherein each R⁸ is hydrogen.
18. The cationic lipid of any one of numbered embodiments 1-4, 7-11, or 16, wherein each R⁸ is methyl.
19. The cationic lipid of any one of numbered embodiments 1-4, 7-11, or 16, wherein Z¹

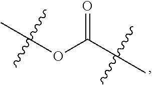 [SMILES: *OC(*)=O]

wherein the left hand side of the depicted structure is bound to the —(CH₂)_{b}—, and Z² is —S—S—, preferably wherein the cationic lipid has a structure according to any one of Formula (IA) or Formula (IA1).
20. The cationic lipid of any one of numbered embodiments 1-4, 7-11, or 16, wherein Z¹ and Z² are both —S—S—, preferably wherein the cationic lipid has a structure according to any one of Formula (IA) or Formula (IA1).
21. The cationic lipid of any one of numbered embodiments 1-4, 7-11, or 16, wherein Z¹ and Z² are both a covalent bond, preferably wherein the cationic lipid has a structure according to any one of Formula (IA), Formula (IA1) or Formula (IA2).
22. The cationic lipid of any one of numbered embodiments 1-4, 7-11, or 16, wherein Z¹ is

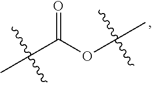 [SMILES: *OC(*)=O]

wherein the left hand side of the depicted structure is bound to the —(CH₂)_{b}—, and Z² is

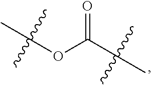 [SMILES: *OC(*)=O]

wherein the right hand side of the depicted structure is bound to the —(CH₂)_{d}—, preferably wherein the cationic lipid has a structure according to any one of Formula (IA), Formula (IA1), Formula (IA2), Formula (IC) or Formula (ID).
23. The cationic lipid of any one of numbered embodiments 1-4, 7-11, or 16 wherein Z¹ is

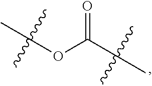 [SMILES: *OC(*)=O]

wherein the left hand side of the depicted structure is bound to the —(CH₂)_{b}—, and Z² is

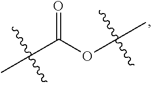 [SMILES: *OC(*)=O]

wherein the right hand side of the depicted structure is bound to the —(CH₂)_{d}—, preferably wherein the cationic lipid has a structure according to any one of Formula (IA2), Formula (IB) or Formula (IE).
24. The cationic lipid of any one of numbered embodiments 1-4, 7-11, or 16, wherein Z¹ is

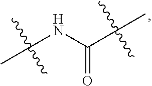 [SMILES: *NC(*)=O]

wherein the left hand side of the depicted structure is bound to the —(CH₂)_{b}—, and Z² is

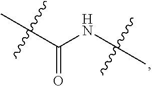 [SMILES: *NC(*)=O]

wherein the right hand side of the depicted structure is bound to the —(CH₂)_{d}—, preferably wherein the cationic lipid has a structure according to any one of Formula (IA) or Formula (IA1).
25. The cationic lipid of any one of numbered embodiments 1-4, 7-11, or 16, wherein Z¹ is

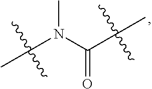 [SMILES: *C(=O)N(*)C]

wherein the left hand side of the depicted structure is bound to the —(CH₂)_{b}—, and Z² is

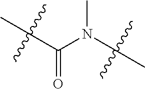 [SMILES: *C(=O)N(*)C]

wherein the right hand side of the depicted structure is bound to the —(CH₂)_{d}—, preferably wherein the cationic lipid has a structure according to any one of Formula (IA) or Formula (IA1).
26. The cationic lipid of any one of numbered embodiments 1-5, 7-12, 14, or 16-25 wherein a is 1 or 2, preferably wherein the cationic lipid has a structure according to any one of Formula (IA), Formula (IA1), or Formula (IA2).
27. The cationic lipid of any one of numbered embodiments 1-5, 7-12, 14, or 16-26, wherein a is 1, preferably wherein the cationic lipid has a structure according to (i) Formula (IB) or (ii) Formula (IA1ii) or Formula (IA1iii).
28. The cationic lipid of any one of numbered embodiments 1-5, 7-12, 14, or 16-26, wherein a is 2, preferably wherein the cationic lipid has a structure according to any one of Formula (IC), Formula (ID) or Formula (IE).
29. The cationic lipid of any one of numbered embodiments 1-28, wherein b is 2, 3 or 4, preferably wherein the cationic lipid has a structure according to any one of Formula (IA), Formula (IA1), or Formula (IA2).
30. The cationic lipid of any one of numbered embodiments 1-29, wherein b is 3 or 4, preferably wherein the cationic lipid has a structure according to (i) Formula (IA1ia) or (ii) Formula (IA1ii), Formula (IA1iia), Formula (IA1iii) or Formula (IA1iiia).
31. The cationic lipid of any one of numbered embodiments 1-30, wherein b is 3, preferably wherein the cationic lipid has a structure according to any one of Formula (IB), Formula (ID), or Formula (IE).
32. The cationic lipid of any one of numbered embodiments 1-30, wherein b is 4, preferably wherein the cationic lipid has a structure according to Formula (IC).
33. The cationic lipid of any one of numbered embodiments 1-5, 7-12, 14, or 16-32, wherein c is 1 or 2, preferably wherein the cationic lipid has a structure according to any one of Formula (IA), Formula (IA1), or Formula (IA2).
34. The cationic lipid of any one of numbered embodiments 1-5, 7-12, 14, or 16-33, wherein c is 1, preferably wherein the cationic lipid has a structure according to (i) Formula (IB) or (ii) Formula (IA1ii) or Formula (IA1iii).
35. The cationic lipid of any one of numbered embodiments 1-5, 7-12, 14, or 16-33, wherein c is 2, preferably wherein the cationic lipid has a structure according to any one of Formula (IC), Formula (ID) or Formula (IE).
36. The cationic lipid of any one of numbered embodiments 1-35, wherein d is 2, 3 or 4, preferably wherein the cationic lipid has a structure according to any one of Formula (IA), Formula (IA1), or Formula (IA2).
37. The cationic lipid of any one of numbered embodiments 1-36, wherein d is 3 or 4, preferably wherein the cationic lipid has a structure according to (i) Formula (IA1ia) or (ii) Formula (IA1ii), Formula (IA1iia), Formula (IA1iii) or Formula (IA1iiia).
38. The cationic lipid of any one of numbered embodiments 1-37, wherein d is 3, preferably wherein the cationic lipid has a structure according to any one of Formula (IB), Formula (ID) or Formula (IE).
39. The cationic lipid of any one of numbered embodiments 1-37, wherein d is 4, preferably wherein the cationic lipid has a structure according to Formula (IC).
40. The cationic lipid of any one of the preceding numbered embodiments, wherein each R^{2A}, R^{2B}, R^{2C} and R^{2D} is independently selected from:

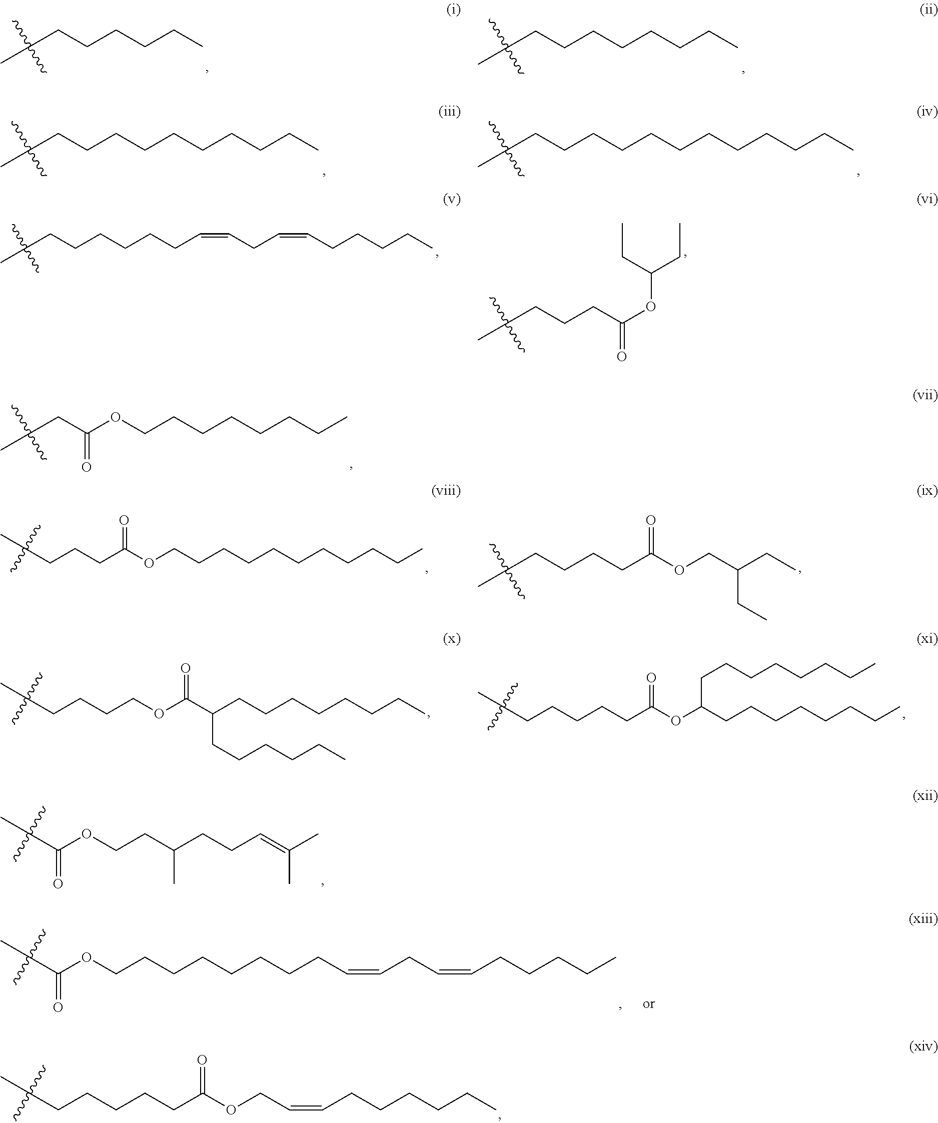

preferably wherein each R^{2A}, R^{2B}, R^{2C} and R^{2D} is independently selected from options (ii), (iii), (iv), (v), (vi), (viii), (ix), (x), (xi), (xii), (xiii) or (xiv).
41. The cationic lipid of any one of numbered embodiments 1-39, wherein each R^{2A}, R^{2B}, R^{2C} and R^{2D} is independently selected from:

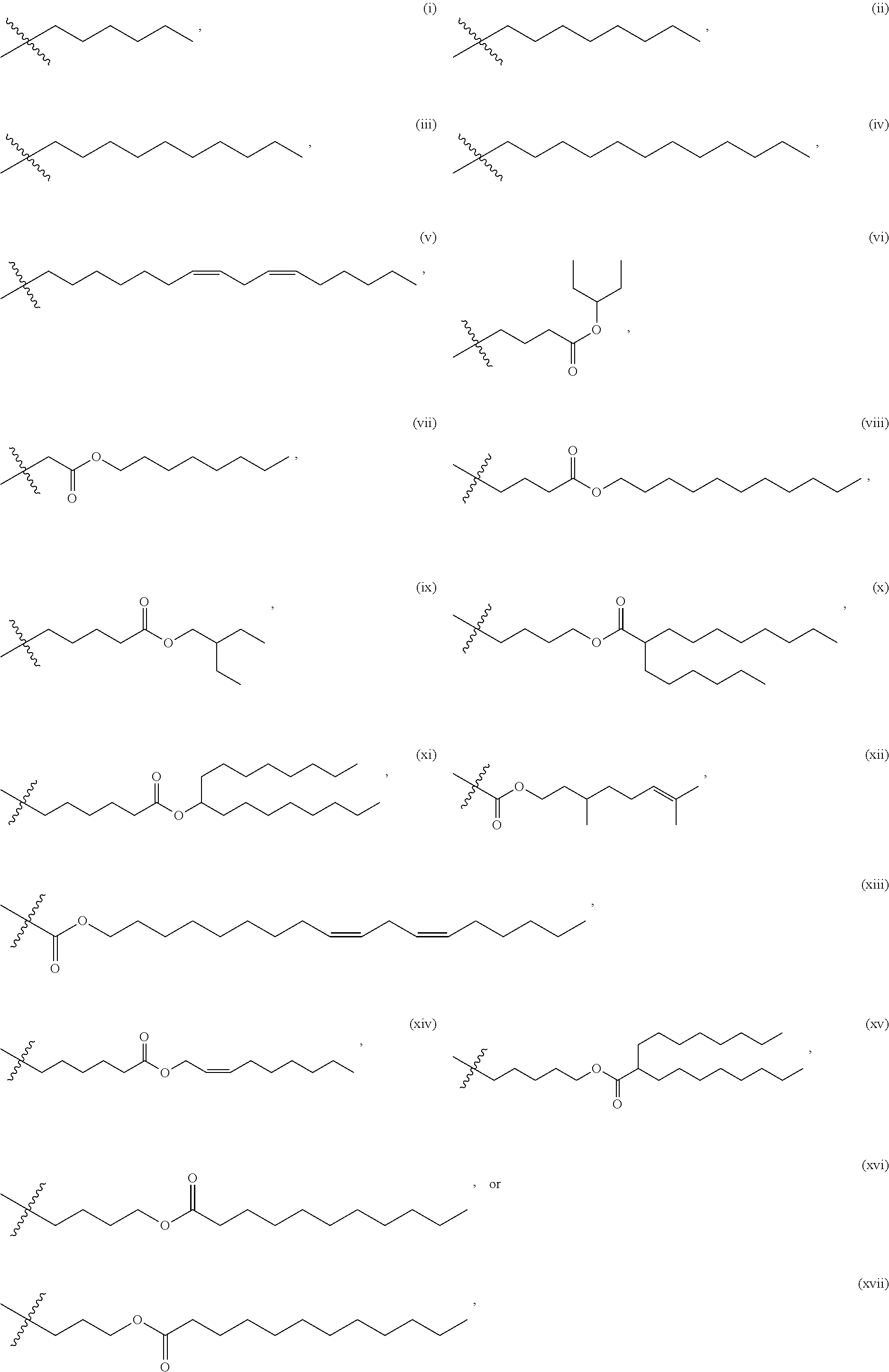

preferably wherein each R^{2A}, R^{2B}, R^{2C} and R^{2D} is independently selected from options (ii), (iii), (iv), (v), (vi), (viii), (ix), (x), (xi), (xii), (xiii), (xiv), (xv), (xvi) or (xvii).
42. The cationic lipid of any one of the preceding numbered embodiments, wherein R^{2A}, R^{2B}, R^{2C} and R^{2D} are the same.
43. The cationic lipid of any one of numbered embodiments 1-41, wherein R^{2A} and R^{2B} are the same and R^{2C} and R^{2D} are the same.
44. The cationic lipid of any one of numbered embodiments 1-41, wherein R^{2A} and R^{2C} are the same and R^{2B} and R^{2D} are the same.
45. The cationic lipid of any one of numbered embodiments 1-41, wherein R^{2A} and R^{2C} are the same and R^{2B} and R^{2D} are different.
46. A cationic lipid having a structure according to Formula (II):

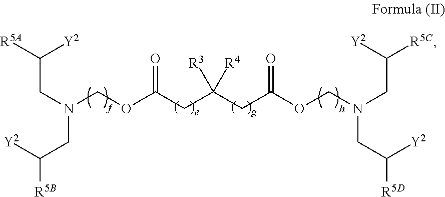

or a pharmaceutically acceptable salt thereof,
   wherein R³ is selected from hydrogen, or optionally substituted (C₁-C₆)alkyl;
   R⁴ is selected from hydrogen, —OH, —NH₂, optionally substituted (C₁-C₆)alkyl, optionally substituted aryl, optionally substituted heteroaryl, optionally substituted (C₁-C₃)alkylene-optionally substituted aryl, or optionally substituted (C₁-C₃)alkylene-optionally substituted heteroaryl;
   e and g are integers that are each independently selected from 0, 1, 2, 3, or 4;
   f and h are integers that are each independently selected from 1, 2, 3, 4, 5 or 6;
   each Y² is independently selected from hydrogen or —OH;
   R^{5A}, R^{5B}, R^{5C} and R^{5D} are each independently selected from optionally substituted (C₅-C₂₅)alkyl, optionally substituted (C₅-C₂₅)alkenyl, or —W¹—X¹;
   each W¹ is independently selected from a covalent bond, optionally substituted (C₁-C₁₀)alkylene or optionally substituted (C₂-C₁₀)alkenylene; and
   each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y²)—when W¹ is a covalent bond.
47. The cationic lipid of numbered embodiment 46, wherein the cationic lipid has a structure according to Formula (IIA):

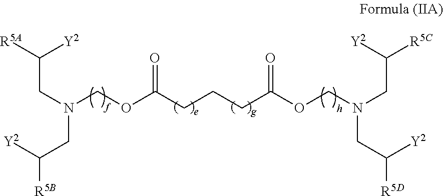

or a pharmaceutically acceptable salt thereof.
48. The cationic lipid of numbered embodiment 46 or 47, wherein the cationic lipid has a structure according to Formula (IIA1):

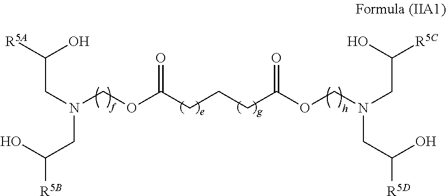

or a pharmaceutically acceptable salt thereof.
49. The cationic lipid of any one of numbered embodiments 46-48, wherein the cationic lipid has a structure according to Formula (IIA1i):

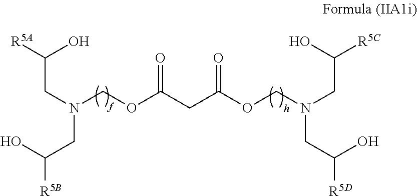

or a pharmaceutically acceptable salt thereof.
50. The cationic lipid of numbered embodiment 46 or 47, wherein the cationic lipid has a structure according to Formula (IIA2):

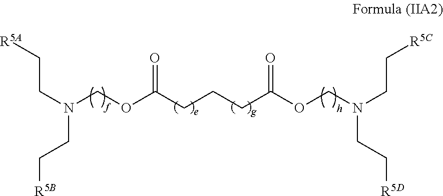

or a pharmaceutically acceptable salt thereof.
51. The cationic lipid of numbered embodiment 46, wherein the cationic lipid has a structure according to Formula (IIB):

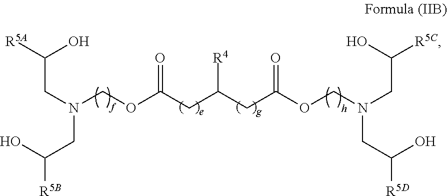

or a pharmaceutically acceptable salt thereof,
wherein R⁴ is selected from (C₁-C₆)alkyl, phenyl or benzyl.
52. The cationic lipid of numbered embodiment 51, wherein R⁴ is selected from methyl, isopropyl, phenyl or benzyl.
53. The cationic lipid of numbered embodiment 52, wherein R⁴ is methyl.
54. The cationic lipid of numbered embodiment 52, wherein R⁴ is isopropyl.
55. The cationic lipid of numbered embodiment 52, wherein R⁴ is phenyl.
56. The cationic lipid of numbered embodiment 52, wherein R⁴ is benzyl.
57. The cationic lipid of numbered embodiment 46, wherein the cationic lipid has a structure according to Formula (IIC):

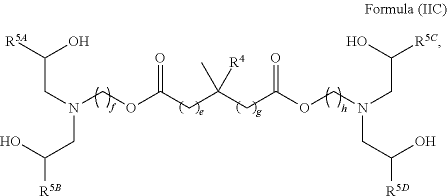

or a pharmaceutically acceptable salt thereof,
wherein R⁴ is selected from (C₁-C₆)alkyl.
58. The cationic lipid of numbered embodiment 57, wherein R⁴ is selected from methyl or ethyl.
59. The cationic lipid of numbered embodiment 58, wherein R⁴ is methyl.
60. The cationic lipid of numbered embodiment 58, wherein R⁴ is ethyl.
61. The cationic lipid of numbered embodiment 46, wherein the cationic lipid has a structure according to Formula (IIC1):

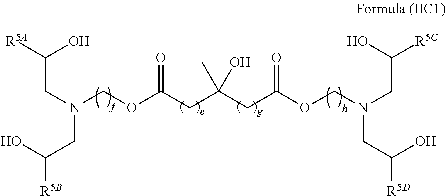

or a pharmaceutically acceptable salt thereof.
62. The cationic lipid of numbered embodiment 46, wherein the cationic lipid has a structure according to Formula (IIC1i):

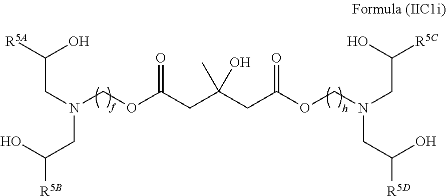

or a pharmaceutically acceptable salt thereof.
63. The cationic lipid of numbered embodiment 46, wherein the cationic lipid has a structure according to Formula (IID):

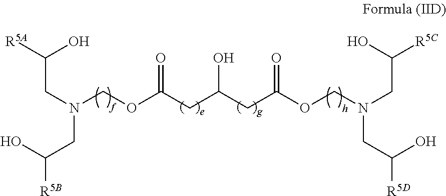

or a pharmaceutically acceptable salt thereof.
64. The cationic lipid of numbered embodiment 46 or 63, wherein the cationic lipid has a structure according to Formula (IIC1):

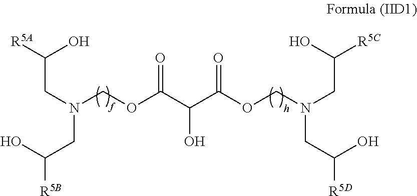

or a pharmaceutically acceptable salt thereof.
65. The cationic lipid of numbered embodiment 46, wherein the cationic lipid has a structure according to Formula (IIE):

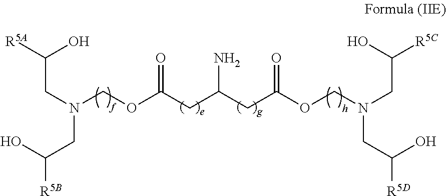

or a pharmaceutically acceptable salt thereof.
66. The cationic lipid of numbered embodiment 46 or 65, wherein the cationic lipid has a structure according to Formula (IIE1):

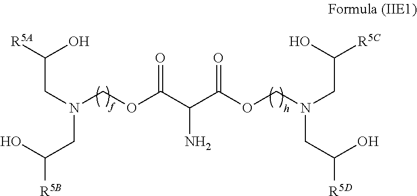

or a pharmaceutically acceptable salt thereof.
67. The cationic lipid of numbered embodiment 46 or 57, wherein the cationic lipid has a structure according to Formula (IIC1ii):

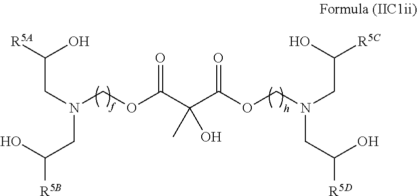

or a pharmaceutically acceptable salt thereof.
68. The cationic lipid of numbered embodiment 46 or 57, wherein the cationic lipid has a structure according to Formula (IIC2):

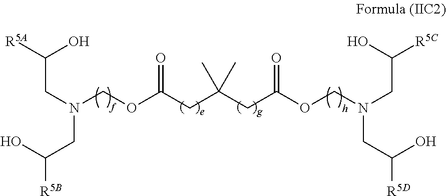

or a pharmaceutically acceptable salt thereof.
69. The cationic lipid of any one of numbered embodiments 46, 57 or 68, wherein the cationic lipid has a structure according to Formula (IIC2i):

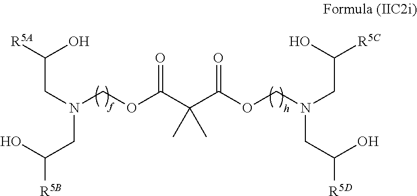

or a pharmaceutically acceptable salt thereof.
70. The cationic lipid of numbered embodiment 46 or 47, wherein Y² is —OH.
71. The cationic lipid of numbered embodiment 46 or 70, wherein R⁴ is selected from hydrogen, —OH, optionally substituted (C₁-C₆)alkyl, optionally substituted phenyl, or optionally substituted (C₁-C₃)alkylene-optionally substituted phenyl.
72. The cationic lipid of any one of numbered embodiments 46-48, 50-61, 63, 65, 68, 70 or 71, wherein e is 0, 1 or 2, preferably wherein the cationic lipid has a structure according to any one of Formula (IIA), Formula (IIA1) or Formula (IIB).
73. The cationic lipid of any one of numbered embodiments 46-48, 50-61, 63, 65, 68 or 70-72, wherein e is 0, preferably wherein the cationic lipid has a structure according to any one of (i) Formula (IIA1i), Formula (IID), or Formula (IIE), or (ii) Formula (IIC1), Formula (IIC2) or Formula (IIE).
74. The cationic lipid of any one of numbered embodiments 46-48, 50-61, 63, 65, 68 or 70-72, wherein e is 1, preferably wherein the cationic lipid has a structure according to any one of Formula (IIA2) or Formula (IIC).
75. The cationic lipid of any one of numbered embodiments 46-74, wherein f is 3, 4, 5 or 6, preferably wherein the cationic lipid has a structure according to Formula (IIC1i).
76. The cationic lipid of any one of numbered embodiments 46-75, wherein f is 3, 4 or 5, preferably wherein the cationic lipid has a structure according to Formula (IIA1i).
77. The cationic lipid of any one of numbered embodiments 46-76, wherein f is 3 or 4, preferably wherein the cationic lipid has a structure according to Formula (II01).
78. The cationic lipid of any one of numbered embodiments 46-77, wherein f is 3, preferably wherein the cationic lipid has a structure according to any one of (i) Formula (IIA), Formula (IIA1), Formula (IIA2), Formula (IIB), Formula (IIC), or Formula (IIE1), or (ii) Formula (IIC1), Formula (IIC1ii), Formula (IIE) or Formula (IIE1).
79. The cationic lipid of any one of numbered embodiments 46-77, wherein f is 4, preferably wherein the cationic lipid has a structure according to Formula (IIC2) or Formula (IIC2i).
80. The cationic lipid of any one of numbered embodiments 46-48, 50-61, 63, 65, 68 or 70-79, wherein g is 0 or 1 preferably wherein the cationic lipid has a structure according to any one of Formula (IIA), Formula (IIA1) or Formula (IIB).
81. The cationic lipid of any one of numbered embodiments 46-48, 50-61, 63, 65, 68 or 70-80, wherein g is 0, preferably wherein the cationic lipid has a structure according to any one of (i) Formula (IIA1i), Formula (IIA2), Formula (IID), or Formula (IIE), or (ii) Formula (IIC1), Formula (IIC2) or Formula (IIE).
82. The cationic lipid of any one of numbered embodiments 46-48, 50-61, 63, 65, 68 or 70-80, wherein g is 1, preferably wherein the cationic lipid has a structure according to Formula (IIC).
83. The cationic lipid of any one of numbered embodiments 46-82, wherein h is 3, 4, 5 or 6, preferably wherein the cationic lipid has a structure according to Formula (IIC1i).
84. The cationic lipid of any one of numbered embodiments 46-83, wherein h is 3, 4 or 5, preferably wherein the cationic lipid has a structure according to Formula (IIA1i).
85. The cationic lipid of any one of numbered embodiments 46-84, wherein h is 3 or 4, preferably wherein the cationic lipid has a structure according to Formula (II01).
86. The cationic lipid of any one of numbered embodiments 46-85, wherein h is 3, preferably wherein the cationic lipid has a structure according to any one of (i) Formula (IIA), Formula (IIA1), Formula (IIA2), Formula (IIB), Formula (IIC), or Formula (IIE1), or (ii) Formula (IIC1), Formula (IIC1ii), Formula (IIE) or Formula (IIE1).
87. The cationic lipid of any one of numbered embodiments 46-85, wherein h is 4, preferably wherein the cationic lipid has a structure according to Formula (IIC2) or Formula (IIC2i).
88. The cationic lipid of any one of numbered embodiments 46-87, wherein each R^{5A}, R^{5B}, R^{5C} and R^{5D} is independently selected from:

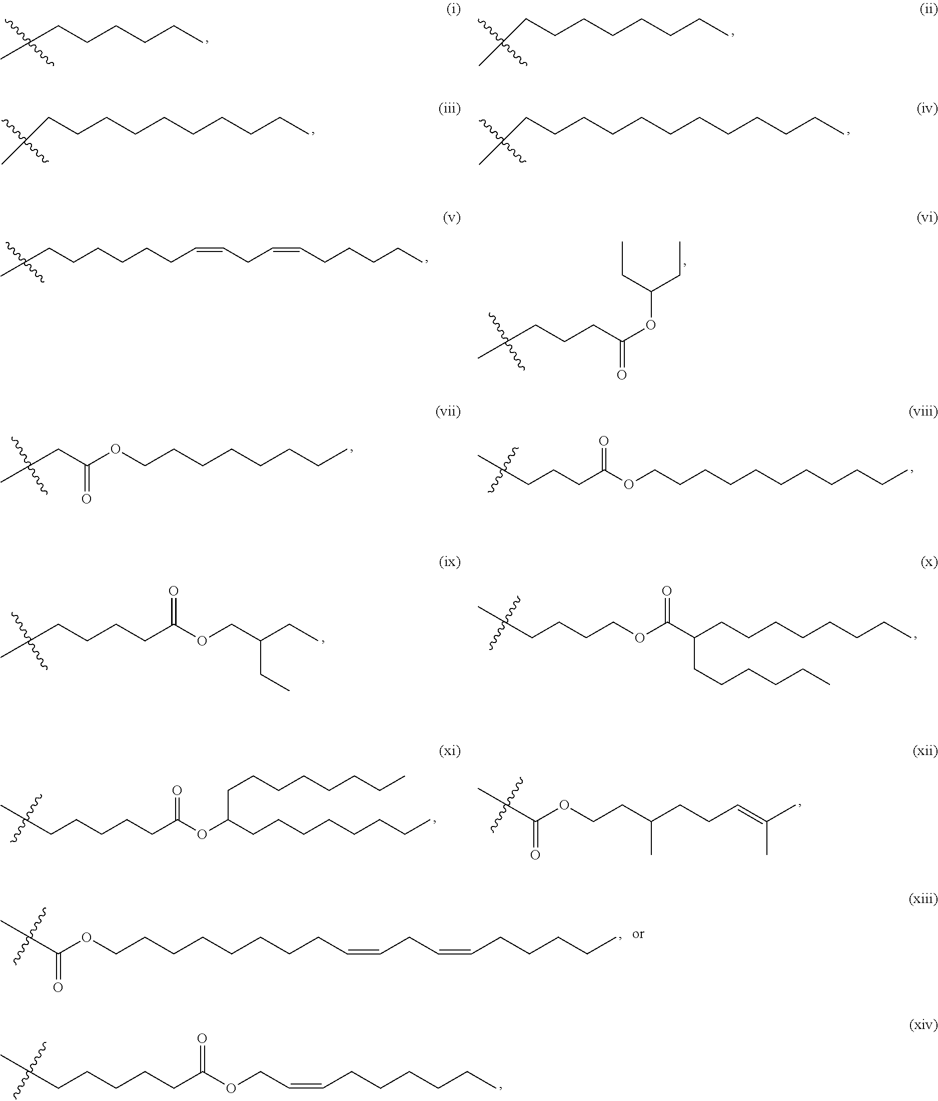

preferably wherein each R^{5A}, R^{5B}, R^{5C} and R^{5D} is independently selected from options (i), (ii), (iii), (vii), (viii), (xi) or (xiv).
89. The cationic lipid of any one of numbered embodiments 46-87, wherein each R^{5A}, R^{5B}, R^{5C} and R^{5D} is independently selected from:

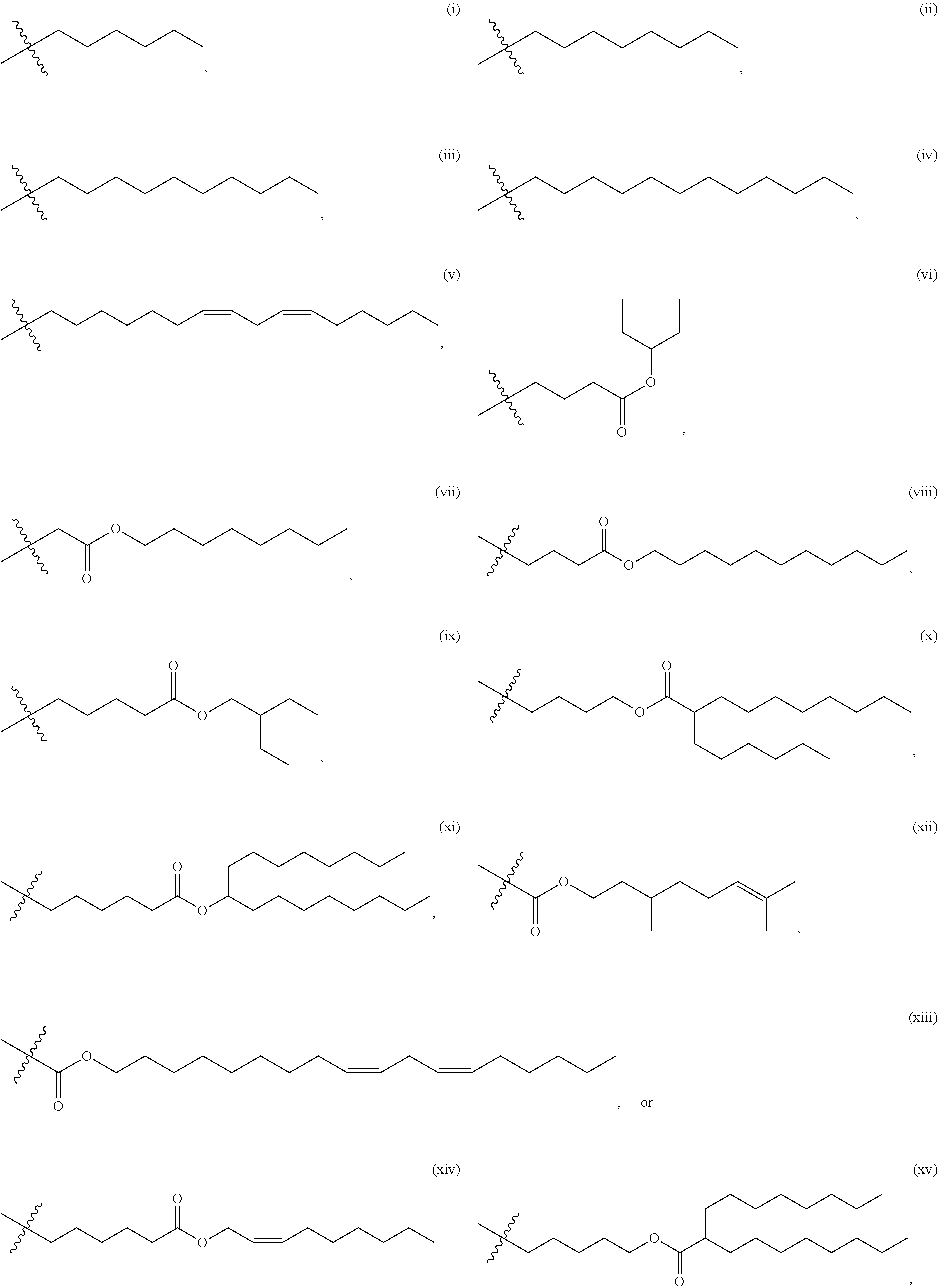

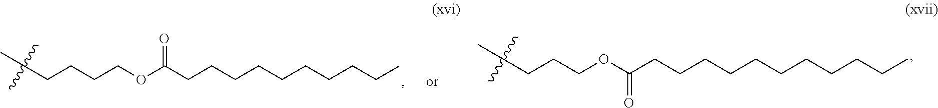 [SMILES: *CCCCOC(=O)CCCCCCCCCC ; *CCCOC(=O)CCCCCCCCCCC]

preferably wherein each R^{5A}, R^{5B}, R^{5C} and R^{5D} is independently selected from options (i), (ii), (iii), (vii), (viii), (xi) or (xiv).
90. The cationic lipid of any one of numbered embodiments 46-89, wherein R^{5A}, R^{5B}, R^{5C} and R^{5D} are the same.
91. The cationic lipid of any one of numbered embodiments 46-89, wherein R^{5A} and R^{5B}are the same and R^{5C} and R^{5D} are the same.
92. The cationic lipid of any one of numbered embodiments 46-89, wherein R^{5A} and R^{5C} are the same and R^{5B} and R^{5D} are the same.
93. A cationic lipid having a structure according to Formula (III):

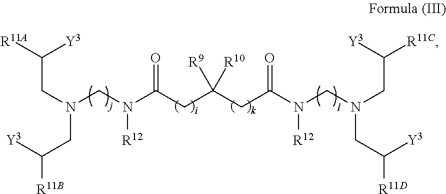

or a pharmaceutically acceptable salt thereof,
   wherein R⁹ is selected from hydrogen, or optionally substituted (C₁-C₆)alkyl;
   R¹⁰ is selected from hydrogen, —OH, —NH₂, optionally substituted (C₁-C₆)alkyl, optionally substituted aryl, optionally substituted heteroaryl, optionally substituted (C₁-C₃)alkylene-optionally substituted aryl, or optionally substituted (C₁-C₃)alkylene-optionally substituted heteroaryl;
   i and k are integers that are each independently selected from 0, 1, 2, 3, or 4;
   j and l are integers that are each independently selected from 1, 2, 3, 4, 5 or 6;
   each Y³ is independently selected from hydrogen or —OH;
   each R¹² is independently selected from hydrogen or optionally substituted (C₁-C₆)alkyl;
   R^{11A}, R^{11B}, R^{11C}, and R^{11D} are each independently selected from optionally substituted (C₅-C₂₅)alkyl, optionally substituted (C₅-C₂₅)alkenyl, or —W¹—X¹;
   each W¹ is independently selected from a covalent bond, optionally substituted (C₁-C₁₀)alkylene or optionally substituted (C₂-C₁₀)alkenylene; and
   each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y³)—when W¹ is a covalent bond.
94. The cationic lipid of numbered embodiment 93, wherein the cationic lipid has a structure according to Formula (IIIA):

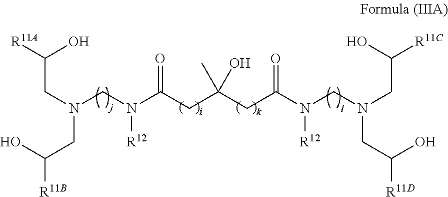

or a pharmaceutically acceptable salt thereof.
95. The cationic lipid of numbered embodiment 93, wherein the cationic lipid has a structure according to Formula (IIIA1):

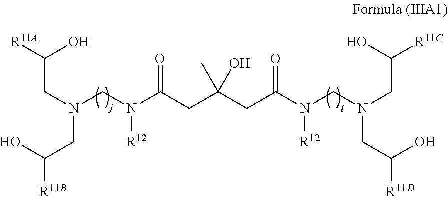

or a pharmaceutically acceptable salt thereof.
96. The cationic lipid of numbered embodiment 93, wherein the cationic lipid has a structure according to Formula (IIIB):

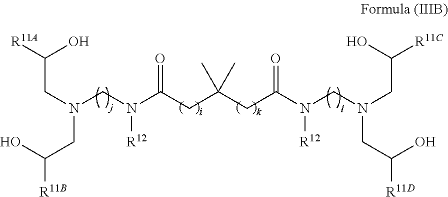

or a pharmaceutically acceptable salt thereof.
97. The cationic lipid of numbered embodiment 93 or 96, wherein the cationic lipid has a structure according to Formula (IIIB1):

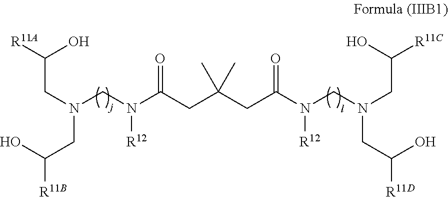

or a pharmaceutically acceptable salt thereof.
98. The cationic lipid of numbered embodiment 93, wherein Y³ is —OH.
99. The cationic lipid of numbered embodiment 93 or 98, wherein R⁹ is methyl.
100. The cationic lipid of any one of numbered embodiments 93, 98 or 99, wherein R¹⁰ is —OH.
101. The cationic lipid of any one of numbered embodiments 93, 98 or 99, wherein R¹⁰ is methyl.
102. The cationic lipid of any one of numbered embodiments 93, 94, 96, or 98-101, wherein i is 1.
103. The cationic lipid of any one of numbered embodiments 93-102, wherein j is 3 or 4.
104. The cationic lipid of any one of numbered embodiments 93-103, wherein j is 3, preferably wherein the cationic lipid has a structure according to any one of Formula (IIIB) or Formula (IIIB1).
105. The cationic lipid of any one of numbered embodiments 93, 94, 96 or 98-104, wherein k is 1.
106. The cationic lipid of any one of numbered embodiments 93-105, wherein I is 3 or 4.
107. The cationic lipid of any one of numbered embodiments 93-106, wherein I is 3, preferably wherein the cationic lipid has a structure according to any one of Formula (IIIB) or Formula (IIIB1).
108. The cationic lipid of any one of numbered embodiments 93-107, wherein each R¹² is hydrogen.
109. The cationic lipid of any one of numbered embodiments 93-107, wherein each R¹² is methyl.
110. The cationic lipid of any one of numbered embodiments 93-109, wherein each R^{11A}, R^{11B}, R^{11C} and R^{11D} is independently selected from:

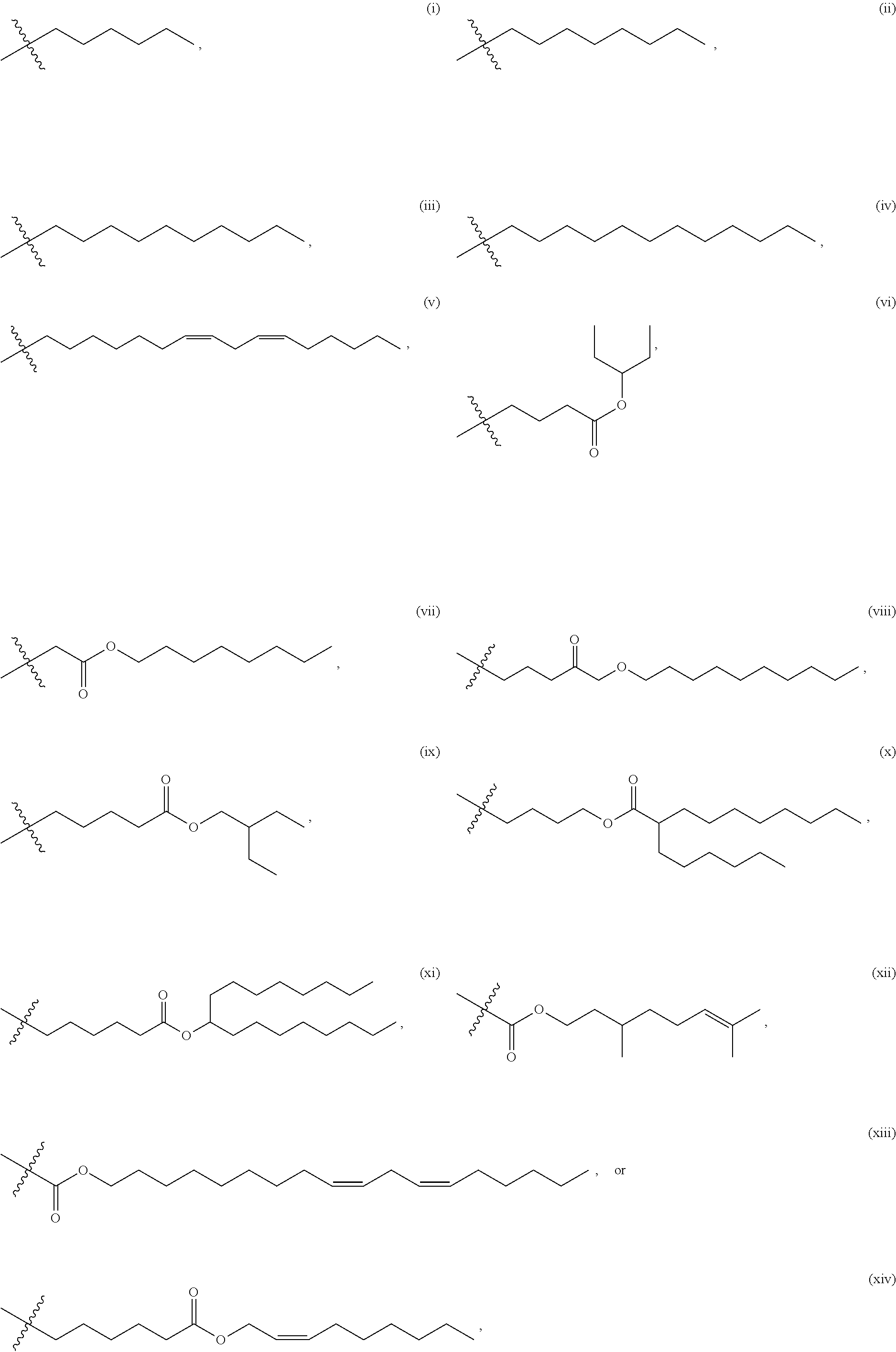

preferably wherein each R^{11A}, R^{11B}, R^{11C} and R^{11D} is independently selected from options (ii) or (iii).
111. The cationic lipid of any one of numbered embodiments 93-109, wherein each R^{11A}, R^{11B}, R^{11C} and R^{11D} is independently selected from:

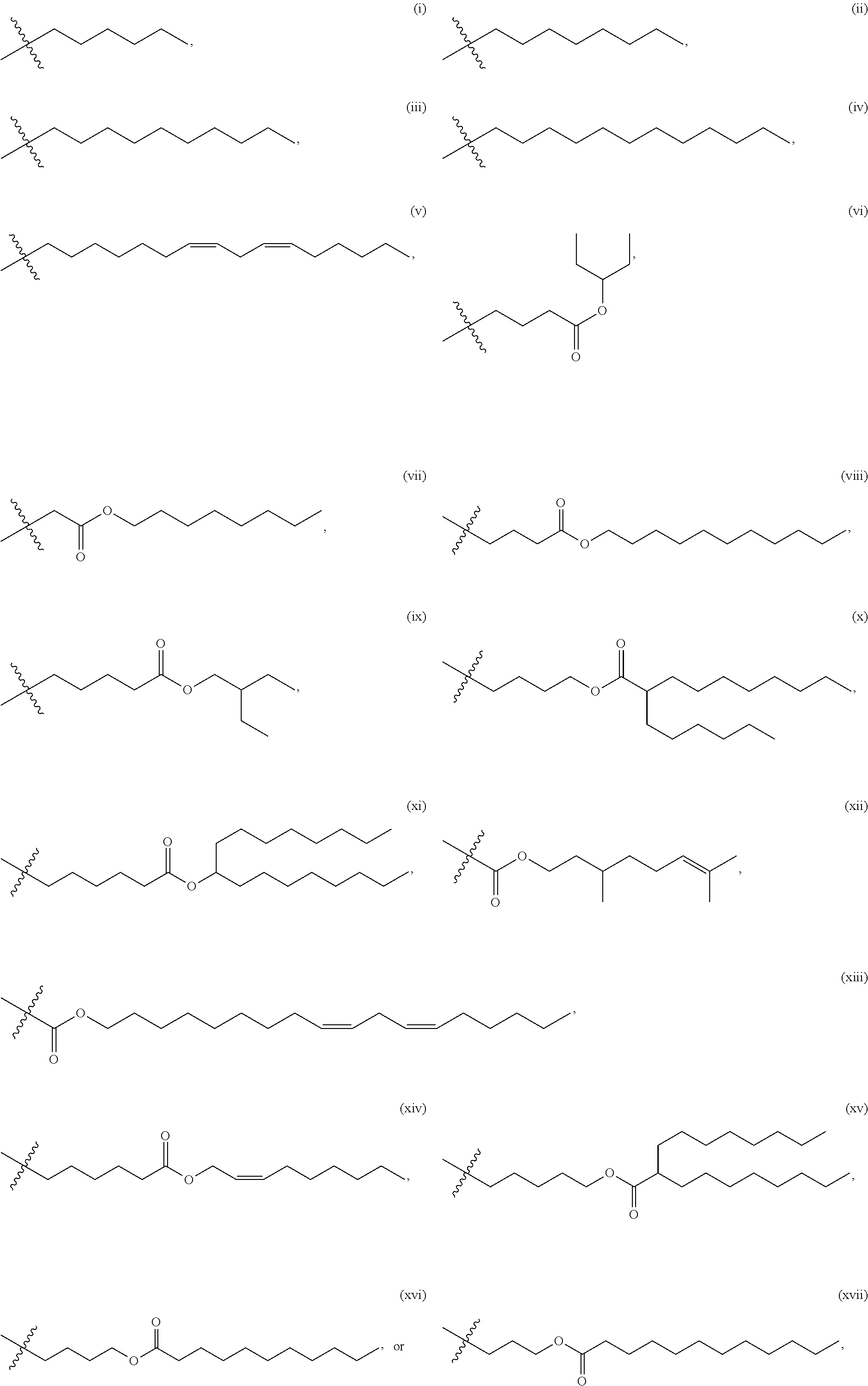

preferably wherein each R^{11A}, R^{11B}, R^{11C} and R^{11D} is independently selected from options (ii), (iii), (viii), (xi), (xiv), (xv), (xvi) or (xvii).
112. The cationic lipid of any one of numbered embodiments 93-111, wherein R^{11A}, R^{11B}, R^{11C} and R^{11D} are the same.
113. The cationic lipid of any one of numbered embodiments 93-111, wherein R^{11A} and R^{11C} are the same and R^{11B} and R^{11D} are the same.
114. The cationic lipid of any one of numbered embodiments 93-111, wherein R^{11A} and R^{11C} are the same and R^{11B} and R^{11D} are different.
115. A cationic lipid having a structure according to Formula (IV):

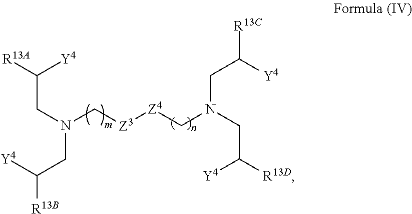

or a pharmaceutically acceptable salt thereof,
wherein
   m and n are integers that are each independently selected from 1, 2, 3, 4, 5 or 6;
   Z³ is an aromatic amino acid residue, wherein the α-carbon carboxyl group (—C(O)O—) of the aromatic amino acid residue is bound to the —(CH₂)ₘ— and the α-carbon aminyl group (—NH—) of the aromatic amino acid residue is bound to the Z⁴;
   Z⁴ is selected from

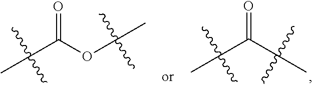

wherein the right hand side of each depicted structure is bound to the —(CH₂)ₙ—;
each Y⁴ is independently selected from hydrogen or —OH;
R^{13A}, R^{13B}, R^{13C} and R^{13D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y⁴)—when W¹ is a covalent bond.
116. The cationic lipid of numbered embodiment 115, wherein the cationic lipid has a structure according to Formula (IVA)

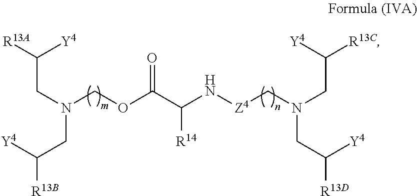

or a pharmaceutically acceptable salt thereof
wherein R¹⁴ is optionally substituted (C₁-C₆)-alkylene-R¹⁵; and
R¹⁵ is selected from optionally substituted aryl or optionally substituted heteroaryl.
117. The cationic lipid of numbered embodiment 115, wherein the cationic lipid has a structure according to
   (i) Formula (IVA1)

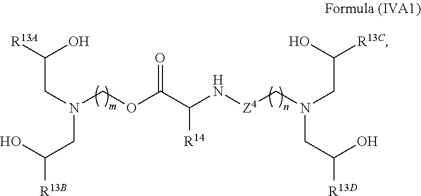

or a pharmaceutically acceptable salt thereof
wherein R¹⁴ is optionally substituted (C₁-C₆)-alkylene-R¹⁵; and
R¹⁵ is selected from optionally substituted aryl or optionally substituted heteroaryl; or
(ii) Formula (IVA2)

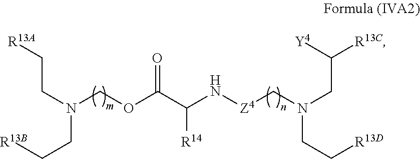

or a pharmaceutically acceptable salt thereof
wherein R¹⁴ is optionally substituted (C₁-C₆)-alkylene-R¹⁵; and
R¹⁵ is selected from optionally substituted aryl or optionally substituted heteroaryl.
118. The cationic lipid of numbered embodiment 115 or 116, wherein Y⁴ is —OH.
119. The cationic lipid of any one of numbered embodiments 115-118, wherein m is 4.
120. The cationic lipid of any one of numbered embodiments 115-118, wherein m is 3.
121. The cationic lipid of any one of numbered embodiments 115-120, wherein n is 4 or 5.
122. The cationic lipid of any one of numbered embodiments 115-120, wherein n is 3.
123. The cationic lipid of any one of numbered embodiments 115-122, wherein Z⁴ is

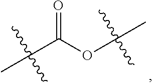 [SMILES: *OC(*)=O]

wherein the right hand side of the depicted structure is bound to the —(CH₂)ₙ—.
124. The cationic lipid of any one of numbered embodiments 115-122, wherein Z⁴ is

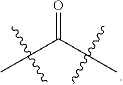 [SMILES: *C(*)=O]

125. The cationic lipid of any one of numbered embodiments 116-124, wherein R¹⁴ is selected from —(CH₂)-optionally substituted aryl, —(CH₂)₂-optionally substituted aryl, —(CH₂)-optionally substituted heteroaryl or —(CH₂)₂-optionally substituted heteroaryl.
126. The cationic lipid of any one of numbered embodiments 116-125, wherein R¹⁴ is selected from —(CH₂)-optionally substituted phenyl, —(CH₂)₂-optionally substituted phenyl, —(CH₂)-optionally substituted imidazolyl or —(CH₂)₂-optionally substituted imidazolyl.
127. The cationic lipid of any one of numbered embodiments 116-125, wherein R¹⁴ is selected from —(CH₂)-optionally substituted phenyl, —(CH₂)₂-optionally substituted phenyl, —(CH₂)-optionally substituted imidazolyl, —(CH₂)₂-optionally substituted imidazolyl or —(CH₂)-optionally substituted indolyl.
128. The cationic lipid of any one of numbered embodiments 116-127, wherein R¹⁴ is selected from

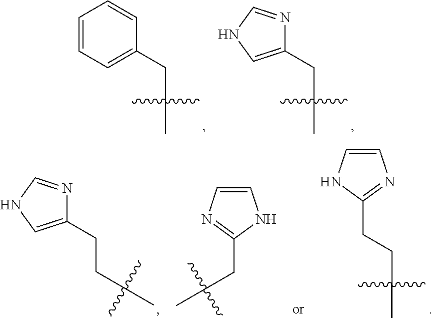 [SMILES: *Cc1ccccc1 ; *Cc1c[nH]cn1 ; *CCc1c[nH]cn1 ; *Cc1ncc[nH]1 ; *CCc1ncc[nH]1]

129. The cationic lipid of any one of numbered embodiments 116-127, wherein R¹⁴ is selected from

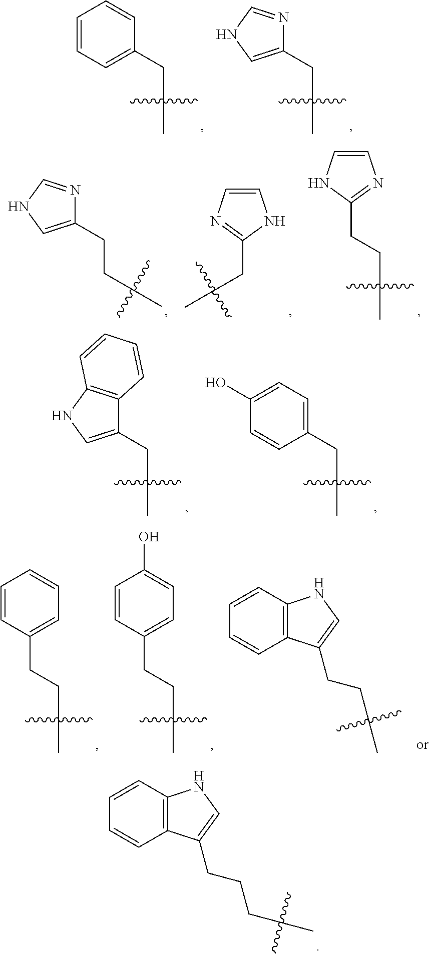

130. The cationic lipid of any one of numbered embodiments 115-129, wherein each R^{13A}, R^{13B}, R^{13C} and R^{13D} is independently selected from:

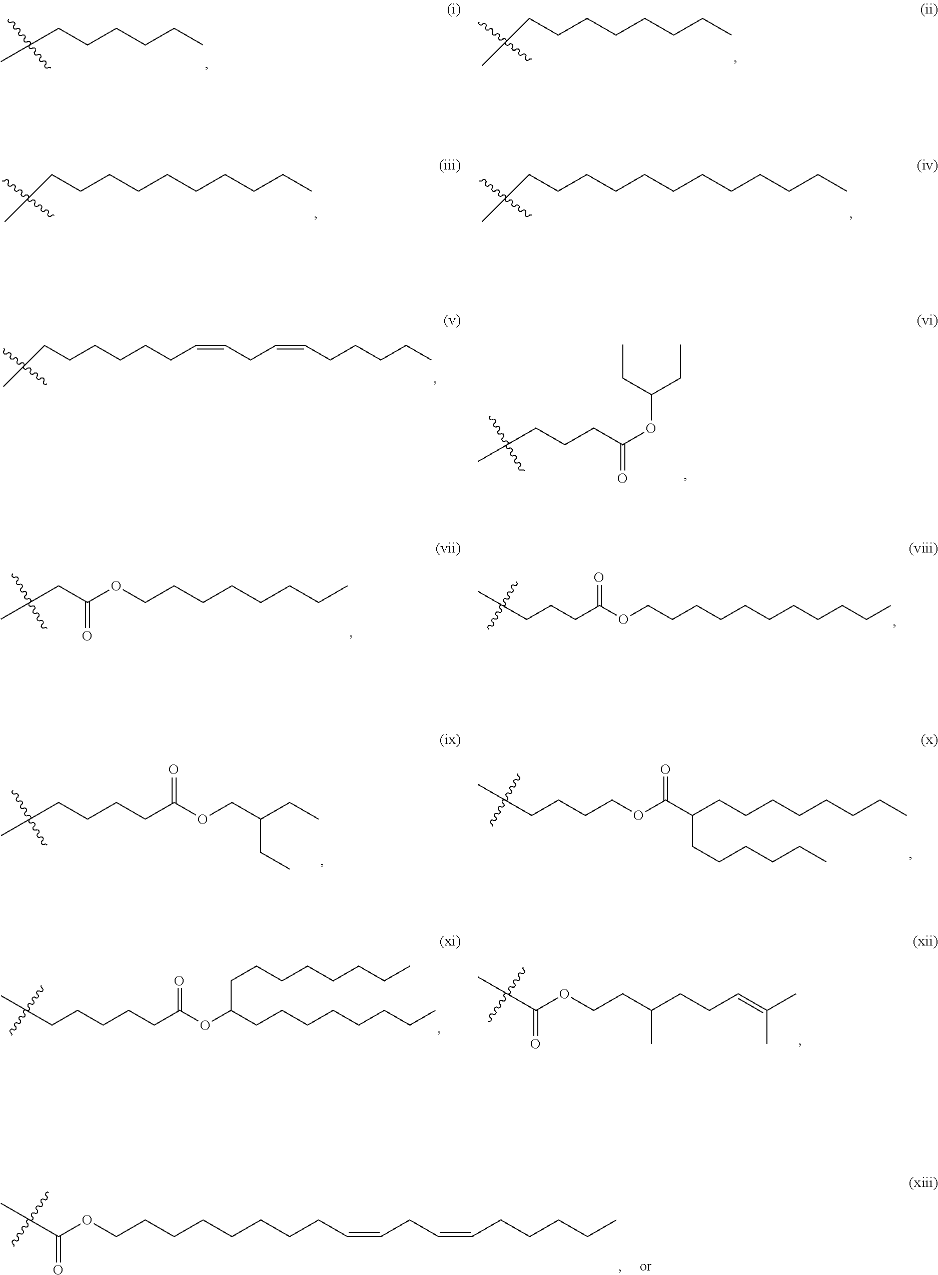

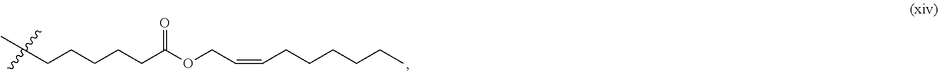 [SMILES: *CCCCCC(=O)OC/C=C\CCCCCC]

preferably wherein each R^{13A}, R^{13B}, R^{13C} and R^{13D} is option (ii).
131. The cationic lipid of any one of numbered embodiments 115-129, wherein each R^{13A}, R^{13B}, R^{13C} and R^{13D} is independently selected from:

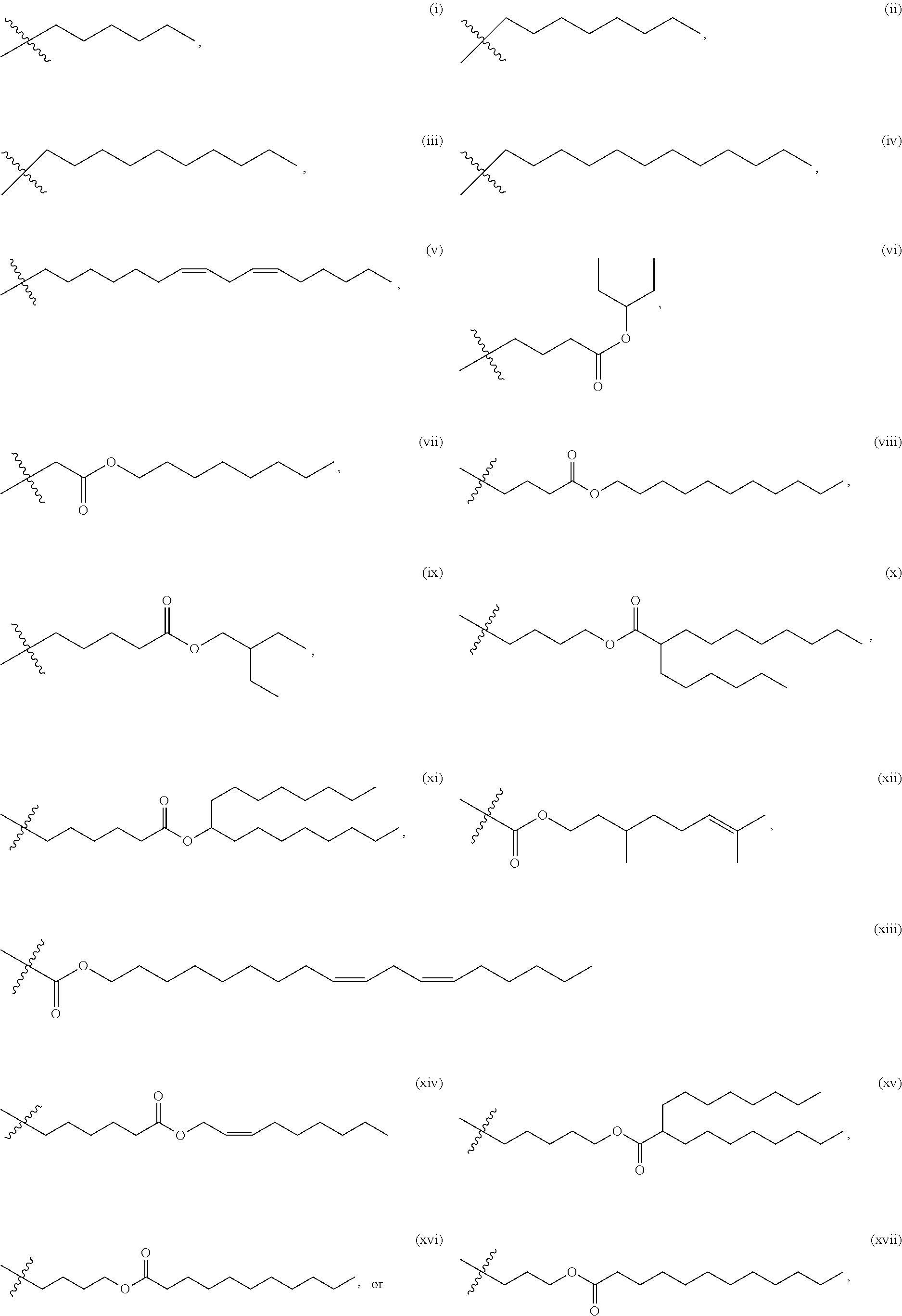

preferably wherein each R^{13A}, R^{13B}, R^{13C} and R^{13D} is option (ii), (iii), (viii), (Xi), (xiv) or (xv).
132. The cationic lipid of any one of numbered embodiments 115-131, wherein R^{13A}, R^{13B}, R^{13C} and R^{13D} are the same.
133. A compound selected from those listed in Table A or a pharmaceutically acceptable salt thereof.
134. A compound selected from those listed in Table B or a pharmaceutically acceptable salt thereof.
135. A composition comprising the cationic lipid of any one of the preceding numbered embodiments and further comprising
   (i) one or more non-cationic lipids,
   (ii) one or more cholesterol-based lipids, and
   (iii) one or more PEG-modified lipids.
136. The composition of numbered embodiment 135, wherein the composition is a lipid nanoparticle, optionally a liposome.
137. The composition of numbered embodiment 136, wherein the one or more cationic lipid(s) constitute(s) about 30 mol %-60 mol % of the lipid nanoparticle.
138. The composition of numbered embodiment 136 or 137, wherein the one or more non-cationic lipid(s) constitute(s) 10 mol %-50 mol % of the lipid nanoparticle.
139. The composition of any one of numbered embodiments 136-138, wherein the one or more PEG-modified lipid(s) constitute(s) 1 mol %-10 mol % of the lipid nanoparticle.
140. The composition of any one of numbered embodiments 136-139, wherein the cholesterol-based lipid constitutes 10 mol %-50 mol % of the lipid nanoparticle.
141. The composition of any one of numbered embodiments 136-140, wherein the lipid nanoparticle encapsulates a nucleic acid, optionally an mRNA encoding a peptide or protein.
142. The composition of any one of numbered embodiments 136-141, wherein the lipid nanoparticle encapsulates an mRNA encoding a peptide or protein.
143. The composition of numbered embodiment 142, wherein the lipid nanoparticles have an encapsulation percentage for mRNA of
   (a) at least 70%;
   (b) at least 75%;
   (c) at least 80%;
   (d) at least 85%;
   (e) at least 90%; or
   (f) at least 95%.
144. The composition of any one of numbered embodiments 141-143 for use in a vaccine.
145. The composition of any one of numbered embodiments 141-144 for use in therapy.
146. The composition of any one of numbered embodiments 142-143 for use in a method of treating or preventing a disease amenable to treatment or prevention by the peptide or protein encoded by the mRNA, optionally wherein the disease is (a) a protein deficiency, optionally wherein the protein deficiency affects the liver, lung, brain or muscle, (b) an autoimmune disease, (c) an infectious disease, or (d) cancer.
147. The composition for use according to any one of numbered embodiments 144-146, wherein the composition is administered intranasally, intravenously, intrathecally or intramuscularly, or by pulmonary delivery, optionally through nebulization.
148. The composition for use according to any one of numbered embodiments 144-146, wherein the composition is administered intramuscularly.
149. A method for treating or preventing a disease wherein said method comprises administering to a subject in need thereof the composition of any one of numbered embodiments 142-143 and wherein the disease is amenable to treatment or prevention by the peptide or protein encoded by the mRNA, optionally wherein the disease is (a) a protein deficiency, optionally wherein the protein deficiency affects the liver, lung, brain or muscle, (b) an autoimmune disease, (c) an infectious disease, or (d) cancer.
150. The method of numbered embodiment 149, wherein the composition is administered intranasally, intravenously, intrathecally or intramuscularly, or by pulmonary delivery, optionally through nebulization.
151. The method of numbered embodiment 149, wherein the composition is administered intramuscularly.

1. A cationic lipid having a structure according to Formula (I): 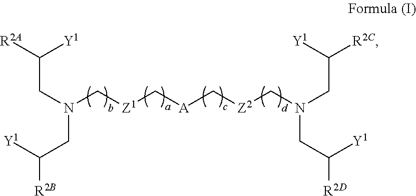
or a pharmaceutically acceptable salt thereof,
wherein A is selected from —N(R¹)— or —S—S—;
R¹ is optionally substituted (C₁-C₆)alkyl;
a and c are integers that are each independently selected from 1, 2, 3 or 4;
b and d are integers that are each independently selected from 1, 2, 3, 4, 5 or 6;
Z¹ is selected from a covalent bond, 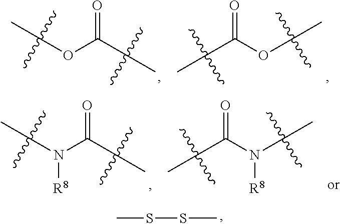
wherein the left hand side of each depicted structure is bound to the —(CH₂)_{b}—;
Z² is selected from a covalent bond, 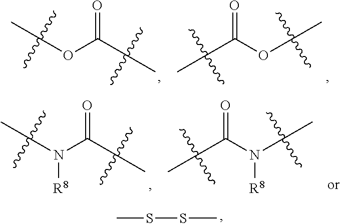
wherein the right hand side of each depicted structure is bound to the —(CH₂)_{d}—;
each Y¹ is independently selected from hydrogen or —OH;
each R³ is independently selected from hydrogen or optionally substituted (C₁-C₆)alkyl;
R^{2A}, R^{2B}, R^{2C}, and R^{2D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y¹)—when W¹ is a covalent bond.

2. A cationic lipid having a structure according to Formula (II): 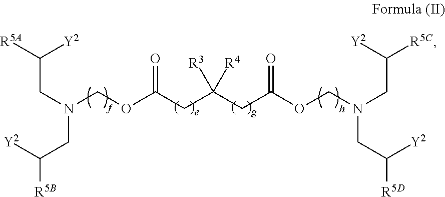
or a pharmaceutically acceptable salt thereof,
wherein R³ is selected from hydrogen, or optionally substituted (C₁-C₆)alkyl;
R⁴ is selected from hydrogen, —OH, —NH₂, optionally substituted (C₁-C₆)alkyl, optionally substituted aryl, optionally substituted heteroaryl, optionally substituted (C₁-C₃)alkylene-optionally substituted aryl, or optionally substituted (C₁-C₃)alkylene-optionally substituted heteroaryl;
e and g are integers that are each independently selected from 0, 1, 2, 3, or 4;
f and h are integers that are each independently selected from 1, 2, 3, 4, 5 or 6;
each Y² is independently selected from hydrogen or —OH;
R^{5A}, R^{5B}, R^{5C}, and R^{5D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y²)—when W¹ is a covalent bond.

3. A cationic lipid having a structure according to Formula (III): 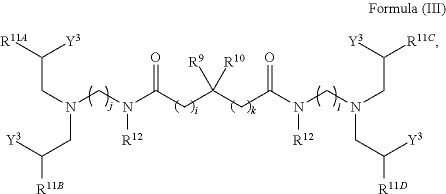
or a pharmaceutically acceptable salt thereof,
wherein R⁹ is selected from hydrogen, or optionally substituted (C₁-C₆)alkyl;
R¹⁰ is selected from hydrogen, —OH, —NH₂, optionally substituted (C₁-C₆)alkyl, optionally substituted aryl, optionally substituted heteroaryl, optionally substituted (C₁-C₃)alkylene-optionally substituted aryl, or optionally substituted (C₁-C₃)alkylene-optionally substituted heteroaryl;
i and k are integers that are each independently selected from 0, 1, 2, 3, or 4;
j and l are integers that are each independently selected from 1, 2, 3, 4, 5 or 6;
each Y³ is independently selected from hydrogen or —OH;
each R¹² is independently selected from hydrogen or optionally substituted (C₁-C₆)alkyl;
R^{11A}, R^{11B}, R^{11C}, and R^{11D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y³)—when W¹ is a covalent bond.

4. A cationic lipid having a structure according to Formula (IV): 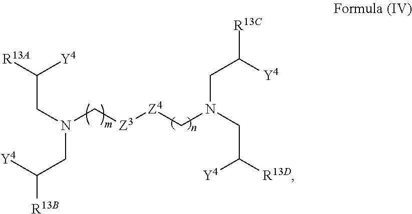
or a pharmaceutically acceptable salt thereof,
wherein
m and n are integers that are each independently selected from 1, 2, 3, 4, 5 or 6;
Z³ is an aromatic amino acid residue, wherein the α-carbon carboxyl group (—C(O)O—) of the aromatic amino acid residue is bound to the —(CH₂)ₘ and the α-carbon aminyl group (—NH—) of the aromatic amino acid residue is bound to the Z⁴;
Z⁴ is selected from 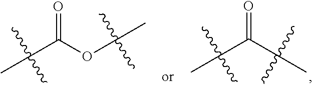
or wherein the right hand side of each depicted structure is bound to the —(CH₂)ₙ—;
each Y⁴ is independently selected from hydrogen or —OH;
R^{13A}, R^{13B}, R^{13C}, and R^{13D} are each independently selected from optionally substituted (C₅-C₂₅)alkyl, optionally substituted (C₅-C₂₅)alkenyl, or —W¹—X¹;
each W¹ is independently selected from a covalent bond, optionally substituted (C₁-C₁₀)alkylene or optionally substituted (C₂-C₁₀)alkenylene; and
each X¹ is independently selected from —(*C═O)—O-optionally substituted (C₃-C₂₅)alkyl, —(*C═O)—O-optionally substituted (C₃-C₂₅)alkenyl, —*O—(C═O)-optionally substituted (C₃-C₂₅)alkyl, or —*O—(C═O)-optionally substituted (C₃-C₂₅)alkenyl, wherein the atom marked with a * is connected to W¹ or —CH(Y⁴)—when W¹ is a covalent bond.

5. A compound selected from those listed in (i) Table A or a pharmaceutically acceptable salt thereof or (ii) Table B or a pharmaceutically acceptable salt thereof.

6. A composition comprising the cationic lipid of any one of the preceding claims and further comprising
(i) one or more non-cationic lipids,
(ii) one or more cholesterol-based lipids, and
(iii) one or more PEG-modified lipids.

7. The composition of claim 6, wherein the composition is a lipid nanoparticle, optionally a liposome.

8. The composition of claim 7, wherein the lipid nanoparticle encapsulates a nucleic acid, optionally an mRNA encoding a peptide or protein.

9. The composition of claim 7 or 8, wherein the lipid nanoparticle encapsulates an mRNA encoding a peptide or protein.

10. The composition of claim 8 or 9 for use in a vaccine.

11. The composition of any one of claims 8-10 for use in therapy.

12. The composition of claim 9 for use in a method of treating or preventing a disease amenable to treatment or prevention by the peptide or protein encoded by the mRNA, optionally wherein the disease is (a) a protein deficiency, optionally wherein the protein deficiency affects the liver, lung, brain or muscle, (b) an autoimmune disease, (c) an infectious disease, or (d) cancer.

13. The composition for use according to any one of claims 10-12, wherein the composition is administered intranasally, intravenously, intrathecally or intramuscularly, or by pulmonary delivery, optionally through nebulization.

14. A method for treating or preventing a disease wherein said method comprises administering to a subject in need thereof the composition of claim 9 and wherein the disease is amenable to treatment or prevention by the peptide or protein encoded by the mRNA, optionally wherein the disease is (a) a protein deficiency, optionally wherein the protein deficiency affects the liver, lung, brain or muscle, (b) an autoimmune disease, (c) an infectious disease, or (d) cancer.

15. The method of claim 14, wherein the composition is administered intranasally, intravenously, intrathecally or intramuscularly, or by pulmonary delivery, optionally through nebulization.
